SEQUENCE LISTING (561 sequences)

SEQ ID NO: 1 - RNA, 23 nt, Homo sapiens
aaaagcuggguugagagggcgaa

SEQ ID NO: 2 - RNA, 24 nt, Homo sapiens
aaaagugcuuacagugcagguagc

SEQ ID NO: 3 - RNA, 23 nt, Homo sapiens
aaacaaacauggugcacuucuuu

SEQ ID NO: 4 - RNA, 23 nt, Homo sapiens
aaaccguuaccauuacugaguuu

SEQ ID NO: 5 - RNA, 23 nt, Homo sapiens
aaagugcugcgacauuugagcgu

SEQ ID NO: 6 - RNA, 22 nt, Homo sapiens
aaagugcuguucgugcagguag

SEQ ID NO: 7 - RNA, 21 nt, Homo sapiens
aacaauauccuggugcugagu

SEQ ID NO: 8 - RNA, 21 nt, Homo sapiens
aacaggugacugguuagacaa

SEQ ID NO: 9 - RNA, 22 nt, Homo sapiens
aacauucaaccugucggugagu

SEQ ID NO: 10 - RNA, 23 nt, Homo sapiens
aacauucaacgcugucggugagu

SEQ ID NO: 11 - RNA, 22 nt, Homo sapiens
aacauucauugcugucgguggg

SEQ ID NO: 12 - RNA, 22 nt, Homo sapiens
aaccagcaccccaacuuuggac

SEQ ID NO: 13 - RNA, 22 nt, Homo sapiens
aacccguagauccgaacuugug

SEQ ID NO: 14 - RNA, 22 nt, Homo sapiens
aacccguagauccgaucuugug

SEQ ID NO: 15 - RNA, 21 nt, Homo sapiens
aacgcgcuucccuauagaggg

SEQ ID NO: 16 - RNA, 24 nt, Homo sapiens
aacuggcccucaaagucccgcuuu

SEQ ID NO: 17 - RNA, 21 nt, Homo sapiens
aacuggccuacaaagucccag

SEQ ID NO: 18 - RNA, 22 nt, Homo sapiens
aagcugccaguugaagaacugu

SEQ ID NO: 19 - RNA, 22 nt, Homo sapiens
aaggagcucacagucuauugag

SEQ ID NO: 20 - RNA, 23 nt, Homo sapiens
aaggagcuuacaaucuagcuggg

SEQ ID NO: 21 - RNA, 22 nt, Homo sapiens
aagugcuuccuuuuagaggguu

SEQ ID NO: 22 - RNA, 22 nt, Homo sapiens
aauauaacacagauggccuguu

SEQ ID NO: 23 - RNA, 22 nt, Homo sapiens
aaucauucacggacaacacuuu

SEQ ID NO: 24 - RNA, 24 nt, Homo sapiens
aauccuuggaaccuaggugugagu

SEQ ID NO: 25 - RNA, 22 nt, Homo sapiens
aauccuuugucccugggugaga

SEQ ID NO: 26 - RNA, 22 nt, Homo sapiens
aauugcacgguauccaucugua

SEQ ID NO: 27 - RNA, 22 nt, Homo sapiens
acaagucaggcucuugggaccu

SEQ ID NO: 28 - RNA, 19 nt, Homo sapiens
acacaccugguuaaccucu

SEQ ID NO: 29 - RNA, 21 nt, Homo sapiens
acagcaggcacagacaggcag

SEQ ID NO: 30 - RNA, 21 nt, Homo sapiens
acagucugcugagguuggagc

SEQ ID NO: 31 - RNA, 22 nt, Homo sapiens
acauagaggaaauuccacguuu

SEQ ID NO: 32 - RNA, 22 nt, Homo sapiens
accaucgaccguugauuguacc

SEQ ID NO: 33 - RNA, 21 nt, Homo sapiens
acgagacucuggcaugcuaac

SEQ ID NO: 34 - RNA, 22 nt, Homo sapiens
acuagacugaagcuccuugagg

SEQ ID NO: 35 - RNA, 22 nt, Homo sapiens
acucaaaaugggggcgcuuucc

SEQ ID NO: 36 - RNA, 22 nt, Homo sapiens
acuccagccccacagccucagc

SEQ ID NO: 37 - RNA, 23 nt, Homo sapiens
acuccauuuguuuugaugaugga

SEQ ID NO: 38 - RNA, 20 nt, Homo sapiens
acugcagugaaggcacuugu

SEQ ID NO: 39 - RNA, 24 nt, Homo sapiens
acugggggcuuucgggcucugcgu

SEQ ID NO: 40 - RNA, 29 nt, Homo sapiens
acugugcuaaaauugcagacacuaggacc

SEQ ID NO: 41 - RNA, 23 nt, Homo sapiens
acuuuaacauggaagugcuuucu

SEQ ID NO: 42 - RNA, 22 nt, Homo sapiens
agaauuguggcuggacaucugu

SEQ ID NO: 43 - RNA, 21 nt, Homo sapiens
agacccuggucugcacucuau

SEQ ID NO: 44 - RNA, 22 nt, Homo sapiens
agaggcuggccgugaugaauuc

SEQ ID NO: 45 - RNA, 21 nt, Homo sapiens
agagguaguagguugcauagu

SEQ ID NO: 46 - RNA, 22 nt, Homo sapiens
agaucagaaggugauuguggcu

SEQ ID NO: 47 - RNA, 23 nt, Homo sapiens
agcagcauuguacagggcuauca

SEQ ID NO: 48 - RNA, 23 nt, Homo sapiens
agcagcauuguacagggcuauga

SEQ ID NO: 49 - RNA, 24 nt, Homo sapiens
agcuacaucuggcuacugggucuc

SEQ ID NO: 50 - RNA, 23 nt, Homo sapiens
agcuacauugucugcuggguuuc

SEQ ID NO: 51 - RNA, 22 nt, Homo sapiens
agcucggucugaggccccucag

SEQ ID NO: 52 - RNA, 17 nt, Homo sapiens
agcugguguugugaauc

SEQ ID NO: 53 - RNA, 20 nt, Homo sapiens
aggaauguuccuucuuugcc

SEQ ID NO: 54 - RNA, 23 nt, Homo sapiens
aggaccugcgggacaagauucuu

SEQ ID NO: 55 - RNA, 25 nt, Homo sapiens
aggcaccagccaggcauugcucagc

SEQ ID NO: 56 - RNA, 19 nt, Homo sapiens
aggcacggugucagcaggc

SEQ ID NO: 57 - RNA, 23 nt, Homo sapiens
aggcaguguaguuagcugauugc

SEQ ID NO: 58 - RNA, 22 nt, Homo sapiens
aggcaguguauuguuagcuggc

SEQ ID NO: 59 - RNA, 22 nt, Homo sapiens
aggcggggcgccgcgggaccgc

SEQ ID NO: 60 - RNA, 25 nt, Homo sapiens
agggaucgcgggcggguggcggccu

SEQ ID NO: 61 - RNA, 21 nt, Homo sapiens
agggcccccccucaauccugu

SEQ ID NO: 62 - RNA, 23 nt, Homo sapiens
agugacaucacauauacggcagc

SEQ ID NO: 63 - RNA, 23 nt, Homo sapiens
aguggggaacccuuccaugagga

SEQ ID NO: 64 - RNA, 21 nt, Homo sapiens
agugguuuuacccuaugguag

SEQ ID NO: 65 - RNA, 22 nt, Homo sapiens
auaagacgagcaaaaagcuugu

SEQ ID NO: 66 - RNA, 21 nt, Homo sapiens
aucacauugccagggauuacc

SEQ ID NO: 67 - RNA, 21 nt, Homo sapiens
aucacauugccagggauuucc

SEQ ID NO: 68 - RNA, 21 nt, Homo sapiens
auccuugcuaucugggugcua

SEQ ID NO: 69 - RNA, 21 nt, Homo sapiens
aucgggaaugucguguccgcc

SEQ ID NO: 70 - RNA, 25 nt, Homo sapiens
aucuggagguaagaagcacuuucug

SEQ ID NO: 71 - RNA, 22 nt, Homo sapiens
augcaccugggcaaggauucug

SEQ ID NO: 72 - RNA, 20 nt, Homo sapiens
auuccuagaaauuguucaua

SEQ ID NO: 73 - RNA, 22 nt, Homo sapiens
caaagcgcuccccuuuagaggu

SEQ ID NO: 74 - RNA, 21 nt, Homo sapiens
caaagcgcuucccuuuggagc

SEQ ID NO: 75 - RNA, 24 nt, Homo sapiens
caaagugcuuacagugcagguagu

SEQ ID NO: 76 - RNA, 22 nt, Homo sapiens
caaccuggaggacuccaugcug

SEQ ID NO: 77 - RNA, 22 nt, Homo sapiens
caacggaaucccaaaagcagcu

SEQ ID NO: 78 - RNA, 21 nt, Homo sapiens
caagcucgcuucuaugggucu

SEQ ID NO: 79 - RNA, 25 nt, Homo sapiens
caccaccaggaguggagccugcggc

SEQ ID NO: 80 - RNA, 22 nt, Homo sapiens
cacccguagaaccgaccuugcg

SEQ ID NO: 81 - RNA, 23 nt, Homo sapiens
cacucagccuugagggcacuuuc

SEQ ID NO: 82 - RNA, 22 nt, Homo sapiens
cagcagcaauucauguuuugaa

SEQ ID NO: 83 - RNA, 21 nt, Homo sapiens
cagcagcacacugugguuugu

SEQ ID NO: 84 - RNA, 23 nt, Homo sapiens
cagugcaauaguauugucaaagc

SEQ ID NO: 85 - RNA, 22 nt, Homo sapiens
cagugcaaugaugaaagggcau

SEQ ID NO: 86 - RNA, 22 nt, Homo sapiens
cagugcaauguuaaaagggcau

SEQ ID NO: 87 - RNA, 19 nt, Homo sapiens
cauagcgagaccccgucug

SEQ ID NO: 88 - RNA, 22 nt, Homo sapiens
caucccuugcaugguggagggu

SEQ ID NO: 89 - RNA, 21 nt, Homo sapiens
cauuauuacuuuugguacgcg

SEQ ID NO: 90 - RNA, 22 nt, Homo sapiens
cauugcacuugucucggucuga

SEQ ID NO: 91 - RNA, 22 nt, Homo sapiens
ccacugccccaggugcugcugg

SEQ ID NO: 92 - RNA, 23 nt, Homo sapiens
cccaguguucagacuaccuguuc

SEQ ID NO: 93 - RNA, 23 nt, Homo sapiens
cccaguguuuagacuaucuguuc

SEQ ID NO: 94 - RNA, 18 nt, Homo sapiens
ccggggggaggcgggggc

SEQ ID NO: 95 - RNA, 23 nt, Homo sapiens
ccuaguagguguccaguaagugu

SEQ ID NO: 96 - RNA, 22 nt, Homo sapiens
ccucuagauggaagcacugucu

SEQ ID NO: 97 - RNA, 17 nt, Homo sapiens
ccugucugagcgacgcu

SEQ ID NO: 98 - RNA, 23 nt, Homo sapiens
cgcauccccuagggcauuggugu

SEQ ID NO: 99 - RNA, 22 nt, Homo sapiens
cguguucacagcggaccuugau

SEQ ID NO: 100 - RNA, 22 nt, Homo sapiens
cuacaaagggaagcacuuucuc

SEQ ID NO: 101 - RNA, 24 nt, Homo sapiens
cuauacggccuccuagcuuucccc

SEQ ID NO: 102 - RNA, 21 nt, Homo sapiens
cuccagagggaaguacuuucu

SEQ ID NO: 103 - RNA, 21 nt, Homo sapiens
cuccagagggaugcacuuucu

SEQ ID NO: 104 - RNA, 22 nt, Homo sapiens
cuccauuacacuacccugccuc

SEQ ID NO: 105 - RNA, 22 nt, Homo sapiens
cuccugacuccagguccugugu

SEQ ID NO: 106 - RNA, 23 nt, Homo sapiens
cucuagagggaagcacuuucucu

SEQ ID NO: 107 - RNA, 24 nt, Homo sapiens
cucuagagggaagcgcuuucuguu

SEQ ID NO: 108 - RNA, 24 nt, Homo sapiens
cucuugagggaagcacuuucuguu

SEQ ID NO: 109 - RNA, 21 nt, Homo sapiens
cugaccuaugaauugacagcc

SEQ ID NO: 110 - RNA, 21 nt, Homo sapiens
cugcaaagggaagcccuuucu

SEQ ID NO: 111 - RNA, 22 nt, Homo sapiens
cuggacuuagggucagaaggcc

SEQ ID NO: 112 - RNA, 22 nt, Homo sapiens
cuggacuuggagucagaaggcc

SEQ ID NO: 113 - RNA, 22 nt, Homo sapiens
cuggcccucucugcccuuccgu

SEQ ID NO: 114 - RNA, 24 nt, Homo sapiens
cugguuucacaugguggcuuagau

SEQ ID NO: 115 - RNA, 22 nt, Homo sapiens
cugugcgugugacagcggcuga

SEQ ID NO: 116 - RNA, 22 nt, Homo sapiens
cuuucagucggauguuuacagc

SEQ ID NO: 117 - RNA, 22 nt, Homo sapiens
cuuucagucggauguuugcagc

SEQ ID NO: 118 - RNA, 21 nt, Homo sapiens
gaaggcgcuucccuuuagagc

SEQ ID NO: 119 - RNA, 23 nt, Homo sapiens
gaagugcuucgauuuuggggugu

SEQ ID NO: 120 - RNA, 21 nt, Homo sapiens
gagugccuucuuuuggagcgu

SEQ ID NO: 121 - RNA, 22 nt, Homo sapiens
gagugugcuagaguccucgaag

SEQ ID NO: 122 - RNA, 22 nt, Homo sapiens
gauuccuggaaauacuguucuu

SEQ ID NO: 123 - RNA, 24 nt, Homo sapiens
gauugcucugcgugcggaaucgac

SEQ ID NO: 124 - RNA, 22 nt, Homo sapiens
gcacauuacacggucgaccucu

SEQ ID NO: 125 - RNA, 21 nt, Homo sapiens
gcaguccaugggcauauacac

SEQ ID NO: 126 - RNA, 21 nt, Homo sapiens
gccccugggccuauccuagaa

SEQ ID NO: 127 - RNA, 23 nt, Homo sapiens
gccgagacuagagucacauccug

SEQ ID NO: 128 - RNA, 21 nt, Homo sapiens
gccugcugggguggaaccugg

SEQ ID NO: 129 - RNA, 21 nt, Homo sapiens
gcuacuucacaacaccagggc

SEQ ID NO: 130 - RNA, 22 nt, Homo sapiens
gcugcgcuuggauuucgucccc

SEQ ID NO: 131 - RNA, 23 nt, Homo sapiens
ggaaaccguuaccauuacugagu

SEQ ID NO: 132 - RNA, 21 nt, Homo sapiens
ggcaagaugcuggcauagcug

SEQ ID NO: 133 - RNA, 25 nt, Homo sapiens
ggcggagggaaguagguccguuggu

SEQ ID NO: 134 - RNA, 19 nt, Homo sapiens
ggcggcggcggcgacucug

SEQ ID NO: 135 - RNA, 22 nt, Homo sapiens
ggcuggcucgcgaugucuguuu

SEQ ID NO: 136 - RNA, 19 nt, Homo sapiens
gguccagaggggagauagg

SEQ ID NO: 137 - RNA, 22 nt, Homo sapiens
gucaacacuugcugguuuccuc

SEQ ID NO: 138 - RNA, 22 nt, Homo sapiens
gucauacacggcucuccucucu

SEQ ID NO: 139 - RNA, 24 nt, Homo sapiens
guccaguuuucccaggaaucccuu

SEQ ID NO: 140 - RNA, 22 nt, Homo sapiens
gugaaauguuuaggaccacuag

SEQ ID NO: 141 - RNA, 19 nt, Homo sapiens
gugcauuguaguugcauug

SEQ ID NO: 142 - RNA, 21 nt, Homo sapiens
gugccgccaucuuuugagugu

SEQ ID NO: 143 - RNA, 24 nt, Homo sapiens
gugccuacugagcugaaacacagu

SEQ ID NO: 144 - RNA, 23 nt, Homo sapiens
gugccuacugagcugauaucagu

SEQ ID NO: 145 - RNA, 24 nt, Homo sapiens
gugguuguaguccgugcgagaaua

SEQ ID NO: 146 - RNA, 20 nt, Homo sapiens
guguguggaaaugcuucugc

SEQ ID NO: 147 - RNA, 26 nt, Homo sapiens
guuggaggaugaaaguacggagugau

SEQ ID NO: 148 - RNA, 23 nt, Homo sapiens
guuugcacgggugggccuugucu

SEQ ID NO: 149 - RNA, 21 nt, Homo sapiens
uaaagugcugacagugcagau

SEQ ID NO: 150 - RNA, 23 nt, Homo sapiens
uaaagugcuuauagugcagguag

SEQ ID NO: 151 - RNA, 22 nt, Homo sapiens
uaacacugucugguaaagaugg

SEQ ID NO: 152 - RNA, 22 nt, Homo sapiens
uaacacugucugguaacgaugu

SEQ ID NO: 153 - RNA, 22 nt, Homo sapiens
uaacagucuacagccauggucg

SEQ ID NO: 154 - RNA, 21 nt, Homo sapiens
uaacagucuccagucacggcc

SEQ ID NO: 155 - RNA, 22 nt, Homo sapiens
uaaggugcaucuagugcagaua

SEQ ID NO: 156 - RNA, 22 nt, Homo sapiens
uaauacugccggguaaugaugg

SEQ ID NO: 157 - RNA, 23 nt, Homo sapiens
uaauacugccugguaaugaugac

SEQ ID NO: 158 - RNA, 22 nt, Homo sapiens
uaauacugucugguaaaaccgu

SEQ ID NO: 159 - RNA, 21 nt, Homo sapiens
uaaucucagcuggcaacugug

SEQ ID NO: 160 - RNA, 22 nt, Homo sapiens
uaaugccccuaaaaauccuuau

SEQ ID NO: 161 - RNA, 22 nt, Homo sapiens
uacaguacugugauaacugaag

SEQ ID NO: 162 - RNA, 22 nt, Homo sapiens
uacaguagucugcacauugguu

SEQ ID NO: 163 - RNA, 23 nt, Homo sapiens
uaccacaggguagaaccacggac

SEQ ID NO: 164 - RNA, 23 nt, Homo sapiens
uacccauugcauaucggaguugu

SEQ ID NO: 165 - RNA, 22 nt, Homo sapiens
uacccuguagaaccgaauuugu

SEQ ID NO: 166 - RNA, 23 nt, Homo sapiens
uacccuguagauccgaauuugug

SEQ ID NO: 167 - RNA, 21 nt, Homo sapiens
uagcaccaucugaaaucgguu

SEQ ID NO: 168 - RNA, 23 nt, Homo sapiens
uagcaccauuugaaaucaguguu

SEQ ID NO: 169 - RNA, 20 nt, Homo sapiens
uagcaccauuugaaaucggu

SEQ ID NO: 170 - RNA, 21 nt, Homo sapiens
uagcagcacagaaauauuggc

SEQ ID NO: 171 - RNA, 22 nt, Homo sapiens
uagcagcacauaaugguuugug

SEQ ID NO: 172 - RNA, 22 nt, Homo sapiens
uagcagcacaucaugguuuaca

SEQ ID NO: 173 - RNA, 22 nt, Homo sapiens
uagcagcacguaaauauuggcg

SEQ ID NO: 174 - RNA, 23 nt, Homo sapiens
uagcagcgggaacaguucugcag

SEQ ID NO: 175 - RNA, 22 nt, Homo sapiens
uagcuuaucagacugauguuga

SEQ ID NO: 176 - RNA, 23 nt, Homo sapiens
uaggcagugucauuagcugauug

SEQ ID NO: 177 - RNA, 21 nt, Homo sapiens
uagggugcuuagcuguuaacu

SEQ ID NO: 178 - RNA, 22 nt, Homo sapiens
uauacaagggcaagcucucugu

SEQ ID NO: 179 - RNA, 23 nt, Homo sapiens
uauggcacugguagaauucacug

SEQ ID NO: 180 - RNA, 22 nt, Homo sapiens
uauggcuuuucauuccuaugug

SEQ ID NO: 181 - RNA, 23 nt, Homo sapiens
uauggcuuuuuauuccuauguga

SEQ ID NO: 182 - RNA, 21 nt, Homo sapiens
uauugcacuugucccggccug

SEQ ID NO: 183 - RNA, 22 nt, Homo sapiens
ucaaaacugaggggcauuuucu

SEQ ID NO: 184 - RNA, 20 nt, Homo sapiens
ucaaaugcucagacuccugu

SEQ ID NO: 185 - RNA, 23 nt, Homo sapiens
ucaagagcaauaacgaaaaaugu

SEQ ID NO: 186 - RNA, 28 nt, Homo sapiens
ucacaaugcugacacucaaacugcugac

SEQ ID NO: 187 - RNA, 22 nt, Homo sapiens
ucacagugaaccggucucuuuc

SEQ ID NO: 188 - RNA, 22 nt, Homo sapiens
ucacagugaaccggucucuuuu

SEQ ID NO: 189 - RNA, 22 nt, Homo sapiens
ucaggcucaguccccucccgau

SEQ ID NO: 190 - RNA, 22 nt, Homo sapiens
ucagugcacuacagaacuuugu

SEQ ID NO: 191 - RNA, 22 nt, Homo sapiens
ucagugcaucacagaacuuugu

SEQ ID NO: 192 - RNA, 22 nt, Homo sapiens
ucagugcaugacagaacuuggg

SEQ ID NO: 193 - RNA, 18 nt, Homo sapiens
uccaaggaaggcagcagg

SEQ ID NO: 194 - RNA, 23 nt, Homo sapiens
uccagcaucagugauuuuguuga

SEQ ID NO: 195 - RNA, 21 nt, Homo sapiens
ucccacguuguggcccagcag

SEQ ID NO: 196 - RNA, 17 nt, Homo sapiens
ucccagcggugccucca

SEQ ID NO: 197 - RNA, 23 nt, Homo sapiens
ucccccaggugugauucugauuu

SEQ ID NO: 198 - RNA, 22 nt, Homo sapiens
ucccugagacccuaacuuguga

SEQ ID NO: 199 - RNA, 23 nt, Homo sapiens
ucccugagacccuuuaaccugug

SEQ ID NO: 200 - RNA, 21 nt, Homo sapiens
ucccuguccuccaggagcuca

SEQ ID NO: 201 - RNA, 23 nt, Homo sapiens
uccgucucaguuacuuuauagcc

SEQ ID NO: 202 - RNA, 22 nt, Homo sapiens
uccuguacugagcugccccgag

SEQ ID NO: 203 - RNA, 22 nt, Homo sapiens
uccuucauuccaccggagucug

SEQ ID NO: 204 - RNA, 21 nt, Homo sapiens
ucguaccgugaguaauaaugc

SEQ ID NO: 205 - RNA, 21 nt, Homo sapiens
ucgugucuuguguugcagccg

SEQ ID NO: 206 - RNA, 23 nt, Homo sapiens
ucuacaaagggaagcccuuucug

SEQ ID NO: 207 - RNA, 18 nt, Homo sapiens
ucuacagugcacgugucu

SEQ ID NO: 208 - RNA, 24 nt, Homo sapiens
ucucacacagaaaucgcacccguc

SEQ ID NO: 209 - RNA, 22 nt, Homo sapiens
ucucccaacccuuguaccagug

SEQ ID NO: 210 - RNA, 23 nt, Homo sapiens
ucucuggagggaagcacuuucug

SEQ ID NO: 211 - RNA, 22 nt, Homo sapiens
ucucugggccugugucuuaggc

SEQ ID NO: 212 - RNA, 22 nt, Homo sapiens
ucuggcuccgugucuucacucc

SEQ ID NO: 213 - RNA, 23 nt, Homo sapiens
ucuuggaguaggucauugggugg

SEQ ID NO: 214 - RNA, 24 nt, Homo sapiens
ugaaacauacacgggaaaccucuu

SEQ ID NO: 215 - RNA, 25 nt, Homo sapiens
ugaccgauuucuccugguguucaga

SEQ ID NO: 216 - RNA, 22 nt, Homo sapiens
ugagaacugaauuccauaggcu

SEQ ID NO: 217 - RNA, 22 nt, Homo sapiens
ugagaacugaauuccauggguu

SEQ ID NO: 218 - RNA, 22 nt, Homo sapiens
ugagaugaagcacuguagcuca

SEQ ID NO: 219 - RNA, 23 nt, Homo sapiens
ugaggggcagagagcgagacuuu

SEQ ID NO: 220 - RNA, 21 nt, Homo sapiens
ugagguaggagguuguauagu

SEQ ID NO: 221 - RNA, 22 nt, Homo sapiens
ugagguaguaaguuguauuguu

SEQ ID NO: 222 - RNA, 22 nt, Homo sapiens
ugagguaguagauuguauaguu

SEQ ID NO: 223 - RNA, 22 nt, Homo sapiens
ugagguaguagguuguauaguu

SEQ ID NO: 224 - RNA, 22 nt, Homo sapiens
ugagguaguagguuguaugguu

SEQ ID NO: 225 - RNA, 22 nt, Homo sapiens
ugagguaguagguugugugguu

SEQ ID NO: 226 - RNA, 21 nt, Homo sapiens
ugagguaguaguuuguacagu

SEQ ID NO: 227 - RNA, 21 nt, Homo sapiens
ugagguaguaguuugugcugu

SEQ ID NO: 228 - RNA, 22 nt, Homo sapiens
ugauauguuugauauauuaggu

SEQ ID NO: 229 - RNA, 21 nt, Homo sapiens
ugcugacuccuaguccagggc

SEQ ID NO: 230 - RNA, 22 nt, Homo sapiens
uggaagacuagugauuuuguug

SEQ ID NO: 231 - RNA, 21 nt, Homo sapiens
uggaauguaaagaaguaugua

SEQ ID NO: 232 - RNA, 22 nt, Homo sapiens
uggaauguaaggaagugugugg

SEQ ID NO: 233 - RNA, 22 nt, Homo sapiens
uggacggagaacugauaagggu

SEQ ID NO: 234 - RNA, 18 nt, Homo sapiens
uggagagaaaggcaguuc

SEQ ID NO: 235 - RNA, 23 nt, Homo sapiens
uggagugugacaaugguguuugu

SEQ ID NO: 236 - RNA, 22 nt, Homo sapiens
uggcaguguauuguuagcuggu

SEQ ID NO: 237 - RNA, 23 nt, Homo sapiens
uggcagugucuuagcugguuguu

SEQ ID NO: 238 - RNA, 22 nt, Homo sapiens
uggcucaguucagcaggaacag

SEQ ID NO: 239 - RNA, 19 nt, Homo sapiens
ugguagacuauggaacgua

SEQ ID NO: 240 - RNA, 21 nt, Homo sapiens
ugguucuagacuugccaacua

SEQ ID NO: 241 - RNA, 22 nt, Homo sapiens
uguaaacauccccgacuggaag

SEQ ID NO: 242 - RNA, 22 nt, Homo sapiens
uguaaacauccuacacucagcu

SEQ ID NO: 243 - RNA, 23 nt, Homo sapiens
uguaaacauccuacacucucagc

SEQ ID NO: 244 - RNA, 22 nt, Homo sapiens
uguaaacauccucgacuggaag

SEQ ID NO: 245 - RNA, 20 nt, Homo sapiens
uguaaacauccuugacugga

SEQ ID NO: 246 - RNA, 22 nt, Homo sapiens
uguaacagcaacuccaugugga

SEQ ID NO: 247 - RNA, 23 nt, Homo sapiens
uguaguguuuccuacuuuaugga

SEQ ID NO: 248 - RNA, 21 nt, Homo sapiens
ugucaguuugucaaauacccc

SEQ ID NO: 249 - RNA, 23 nt, Homo sapiens
ugucugcccgcaugccugccucu

SEQ ID NO: 250 - RNA, 21 nt, Homo sapiens
ugugacugguugaccagaggg

SEQ ID NO: 251 - RNA, 23 nt, Homo sapiens
ugugcaaauccaugcaaaacuga

SEQ ID NO: 252 - RNA, 22 nt, Homo sapiens
uguuugcagaggaaacugagac

SEQ ID NO: 253 - RNA, 22 nt, Homo sapiens
uuaaugcuaaucgugauagggg

SEQ ID NO: 254 - RNA, 22 nt, Homo sapiens
uuauaauacaaccugauaagug

SEQ ID NO: 255 - RNA, 22 nt, Homo sapiens
uuaucagaaucuccagggguac

SEQ ID NO: 256 - RNA, 21 nt, Homo sapiens
uucaaguaauccaggauaggc

SEQ ID NO: 257 - RNA, 22 nt, Homo sapiens
uucaaguaauucaggauagguu

SEQ ID NO: 258 - RNA, 22 nt, Homo sapiens
uucacagggaggugucauuuau

SEQ ID NO: 259 - RNA, 21 nt, Homo sapiens
uucacaguggcuaaguuccgc

SEQ ID NO: 260 - RNA, 21 nt, Homo sapiens
uucacaguggcuaaguucugc

SEQ ID NO: 261 - RNA, 24 nt, Homo sapiens
uucuccaaaagaaagcacuuucug

SEQ ID NO: 262 - RNA, 22 nt, Homo sapiens
uucuccaaaagggagcacuuuc

SEQ ID NO: 263 - RNA, 20 nt, Homo sapiens
uugcauagucacaaaaguga

SEQ ID NO: 264 - RNA, 22 nt, Homo sapiens
uugcauauguaggaugucccau

SEQ ID NO: 265 - RNA, 21 nt, Homo sapiens
uugguccccuucaaccagcua

SEQ ID NO: 266 - RNA, 22 nt, Homo sapiens
uugguccccuucaaccagcugu

SEQ ID NO: 267 - RNA, 22 nt, Homo sapiens
uuguacaugguaggcuuucauu

SEQ ID NO: 268 - RNA, 21 nt, Homo sapiens
uugugcuugaucuaaccaugu

SEQ ID NO: 269 - RNA, 22 nt, Homo sapiens
uuuaacauggggguaccugcug

SEQ ID NO: 270 - RNA, 23 nt, Homo sapiens
uuucaagccagggggcguuuuuc

SEQ ID NO: 271 - RNA, 22 nt, Homo sapiens
uuuccuaugcauauacuucuuu

SEQ ID NO: 272 - RNA, 22 nt, Homo sapiens
uuuggcaaugguagaacucaca

SEQ ID NO: 273 - RNA, 22 nt, Homo sapiens
uuuguucguucggcucgcguga

SEQ ID NO: 274 - RNA, 22 nt, Homo sapiens
uuuuugcgauguguuccuaaua

SEQ ID NO: 275 - RNA, 97 nt, Homo sapiens
aaacccacaccacugcauuuuggccaucgaggguuggggcuuggugucaugccccaagauaaccagcaccccaacuuuggacagcauggauuagucu

SEQ ID NO: 276 - RNA, 96 nt, Homo sapiens
aaccauucaaauauaccacaguuuguuuaaccuuuugccuguugguugaagaugccuuucaacaggugacugguuagacaaacugugguauauaca

SEQ ID NO: 277 - RNA, 103 nt, Homo sapiens
aauggguuccuaggaagagguaguagguugcauaguuuuagggcagggauuuugcccacaaggagguaacuauacgaccugcugccuuucuuagggccuuauu

SEQ ID NO: 278 - RNA, 85 nt, Homo sapiens
acagugcagucacccauaaaguagaaagcacuacuaacagcacuggaggguguaguguuuccuacuuuauggaugaguguacugu

SEQ ID NO: 279 - RNA, 84 nt, Homo sapiens
acagugcuugguuccuaguagguguccaguaaguguuugugacauaauuuguuuauugaggaccuccuaucaaucaagcacugu

SEQ ID NO: 280 - RNA, 96 nt, Homo sapiens
accucucuaacaaggugcagagcuuagcugauuggugaacagugauugguuuccgcuuuguucacaguggcuaaguucugcaccugaagagaaggu

SEQ ID NO: 281 - RNA, 86 nt, Homo sapiens
accuuguccucacgguccaguuuucccaggaaucccuuagaugcuaagauggggauuccuggaaauacuguucuugaggucauggu

SEQ ID NO: 282 - RNA, 99 nt, Homo sapiens
acugaacacuguucuaugguuaguuuugcagguuugcauccagcugugugauauucugcugugcaaauccaugcaaaacugacugugguagugaaaagu

SEQ ID NO: 283 - RNA, 98 nt, Homo sapiens
agaaugggcaaaugaacaguaaauuuggaggccuggggcccucccugcugcuggagaaguguuugcacgggugggccuugucuuugaaaggaggugga

SEQ ID NO: 284 - RNA, 136 nt, Homo sapiens
agacgagcgugugggaggucucuguguugggcgucugucugcccgcaugccugccucucuguugcucugaaggaggcaggggcugggccugcagcugccugggcagagcggcuccugcaugggacauggcucuucu

SEQ ID NO: 285 - RNA, 96 nt, Homo sapiens
agagaagcuggacaaguacuggucucagcagauugaggagagcaccacaguggucaucacacagucugcugagguuggagcugcugagaugacacu

SEQ ID NO: 286 - RNA, 80 nt, Homo sapiens
aggacccuuccagagggcccccccucaauccuguugugccuaauucagaggguuggguggaggcucuccugaagggcucu

SEQ ID NO: 287 - RNA, 81 nt, Homo sapiens
agugagcgacuguaaacauccucgacuggaagcugugaagccacagaugggcuuucagucggauguuugcagcugccuacu

SEQ ID NO: 288 - RNA, 55 nt, Homo sapiens
auccuugcuaucugggugcuagugcuggcucaaugcaaugcaccugggcaaggau

SEQ ID NO: 289 - RNA, 88 nt, Homo sapiens
auuagcauuugaggugaaguucuguuauacacucaggcuguggcucucugaaagucagugcaucacagaacuuugucucgaaagcuuu

SEQ ID NO: 290 - RNA, 88 nt, Homo sapiens
auugguucccgcccccuguaacagcaacuccauguggaagugcccacugguuccaguggggcugcuguuaucuggggcgagggccagu

SEQ ID NO: 291 - RNA, 96 nt, Homo sapiens
caaaaagggcuucugucgugaguggcacacguagggcaacucgauugcucugcgugcggaaucgacaucaagagauuucggaagcauaauuuuuug

SEQ ID NO: 292 - RNA, 83 nt, Homo sapiens
caaacccuaggucugcugacuccuaguccagggcucgugauggcuggugggcccugaacgaggggucuggaggccuggguuug

SEQ ID NO: 293 - RNA, 116 nt, Homo sapiens
caacuacagccacuacuacaggaccaucgaggaccugcgggacaagauucuuggugccaccauugagaacgccaggauuguccugcagaucaacaaugcucaacuggcugcagaug

SEQ ID NO: 294 - RNA, 82 nt, Homo sapiens
caagacagagaagccaggucacgucucugcaguuacacagcucacgagugccugcugggguggaaccuggucugucugucug

SEQ ID NO: 295 - RNA, 86 nt, Homo sapiens
caaugcuuugcuagagcugguaaaauggaaccaaaucgccucuucaauggauuugguccccuucaaccagcuguagcuaugcauug

SEQ ID NO: 296 - RNA, 86 nt, Homo sapiens
cacagcugccagugucauuuuugugaucugcagcuaguauucucacuccaguugcauagucacaaaagugaucauuggcaggugug

SEQ ID NO: 297 - RNA, 103 nt, Homo sapiens
caccauggcccuggcugagguaguaguuugugcuguuggucggguugugacauugcccgcuguggagauaacugcgcaagcuacugccuugcuagugcuggug

SEQ ID NO: 298 - RNA, 85 nt, Homo sapiens
caccugcuccucagaucagaaggugauuguggcuuuggguggauauuaaucagccacagcacugccuggucagaaagagcaagug

SEQ ID NO: 299 - RNA, 74 nt, Homo sapiens
cagauacuguaaacauccuacacucucagcuguggaaaguaagaaagcugggagaaggcuguuuacucuuucug

SEQ ID NO: 300 - RNA, 84 nt, Homo sapiens
cagcgcgccggcaccuuggcucuagacugcuuacugcccgggccgcccucaguaacagucuccagucacggccaccgacgccug

SEQ ID NO: 301 - RNA, 73 nt, Homo sapiens
cagcugaacugggaggaucgcuugagcccagggguuugagaccagccugggcaacauagcgagaccccgucug

SEQ ID NO: 302 - RNA, 116 nt, Homo sapiens
cagcugcuggaagguguagguacccucaauggcucaguagccaguguagauccugucuuucguaaucagcagcuacaucuggcuacugggucucugauggcaucuucuagcuucug

SEQ ID NO: 303 - RNA, 74 nt, Homo sapiens
caggaagcugguuucauauggugguuuagauuuaaauagugauugucuagcaccauuugaaaucaguguucuug

SEQ ID NO: 304 - RNA, 59 nt, Homo sapiens
cagguugggaucgguugcaaugcuguguuucuguaugguauugcacuugucccggccug

SEQ ID NO: 305 - RNA, 82 nt, Homo sapiens
cagucacguccccuuaucacuuuuccagcccagcuuugugacuguaaguguuggacggagaacugauaaggguaggugauug

SEQ ID NO: 306 - RNA, 162 nt, Homo sapiens
cagugugugcagacagcacgggguggcgcugcucucaggugcucuggcugcuuggguuccuggcaugcugauuugugacuuaagauuaaaaucacauugccagggauuaccacgcaaccacgaccuuggcugcuccuccagaaaccguggucgcgcucacug

SEQ ID NO: 307 - RNA, 97 nt, Homo sapiens
caugccagggugagguaguaaguuguauuguugugggguagggauauuaggccccaauuagaagauaacuauacaacuuacuacuuucccuggugug

SEQ ID NO: 308 - RNA, 60 nt, Homo sapiens
cauugguccagaggggagauagguuccugugauuuuuccuucuucucuauagaauaaaug

SEQ ID NO: 309 - RNA, 100 nt, Homo sapiens
ccaaagaaagaugcuaaacuauuuuugcgauguguuccuaauauguaauauaaauguauuggggacauuuugcauucauaguuuuguaucaauaauaugg

SEQ ID NO: 310 - RNA, 130 nt, Homo sapiens
ccaagagagcaagcccuucccugcucccucucucacaucccuugcaugguggagggugagcuuucugaaaaccccucccacaugcaggguuugcaggauggcgagccucagcuuuccuugcucucuugug

SEQ ID NO: 311 - RNA, 86 nt, Homo sapiens
ccaaguuucaguucauguaaacauccuacacucagcuguaauacauggauuggcugggagguggauguuuacuucagcugacuugg

SEQ ID NO: 312 - RNA, 112 nt, Homo sapiens
ccaccccgguccugcucccgccccagcagcacacugugguuuguacggcacuguggccacguccaaaccacacugugguguuagagcgagggugggggaggcaccgccgagg

SEQ ID NO: 313 - RNA, 87 nt, Homo sapiens
ccagacuuuuccuagucccugagacccuaacuugugagguauuuuaguaacaucacaagucaggcucuugggaccuaggcggagggg

SEQ ID NO: 314 - RNA, 84 nt, Homo sapiens
ccaggcgcagggcagccccugcccaccgcacacugcgcugccccagacccacugugcgugugacagcggcugaucugugccugg

SEQ ID NO: 315 - RNA, 97 nt, Homo sapiens
ccaguggcgcaauggauaacgcgucugacuacggaucagaagauucuagguucgacuccuggcuggcucgcgaugucuguuuugccacacuugaccc

SEQ ID NO: 316 - RNA, 87 nt, Homo sapiens
ccaugcuguaguguguguaaacauccuacacucucagcugugagcucaagguggcugggagaggguuguuuacuccuucugccaugg

SEQ ID NO: 317 - RNA, 83 nt, Homo sapiens
ccauucaucccuggguggggauuuguugcauuacuuguguucuauauaaaguauugcacuugucccggccuguggaagaaagg

SEQ ID NO: 318 - RNA, 82 nt, Homo sapiens
cccauuggcauaaacccguagauccgaucuuguggugaaguggaccgcacaagcucgcuucuaugggucugugucagugugg

SEQ ID NO: 319 - RNA, 100 nt, Homo sapiens
cccccagaaucugucaggcaccagccaggcauugcucagcccguuucccucugggggagcaaggaguggugcuggguuugucucugcugggguuucuccu

SEQ ID NO: 320 - RNA, 127 nt, Homo sapiens
cccuggauguucucuucacugugggaugagguaguagguuguauaguuuuagggucacacccaccacugggagauaacuauacaaucuacugucuuuccuaacgugauagaaaagucugcauccagg

SEQ ID NO: 321 - RNA, 83 nt, Homo sapiens
ccuacucagaguacauacuucuuuauguacccauaugaacauacaaugcuauggaauguaaagaaguauguauuuuugguagg

SEQ ID NO: 322 - RNA, 106 nt, Homo sapiens
ccucaccuggcugagccgcaguaguucuucaguggcaagcuuuauguccugacccagcuaaagcugccaguugaagaacuguugcccucugccccuggcuucgagg

SEQ ID NO: 323 - RNA, 87 nt, Homo sapiens
ccuccccccguuuuuggcaaugguagaacucacacuggugagguaacaggauccggugguucuagacuugccaacuauggggcgagg

SEQ ID NO: 324 - RNA, 84 nt, Homo sapiens
ccucgcuguucucuauggcuuuuuauuccuaugugauucuacugcucacucauauagggauuggagccguggcgcacggcgggg

SEQ ID NO: 325 - RNA, 83 nt, Homo sapiens
ccugaaucuaaagacaacauuucugcacacacaccagacuauggaagccaguguguggaaaugcuucugcuagauucugaagg

SEQ ID NO: 326 - RNA, 73 nt, Homo sapiens
ccuggcacugagaacugaauuccauaggcugugagcucuagcaaugcccuguggacucaguucuggugcccgg

SEQ ID NO: 327 - RNA, 71 nt, Homo sapiens
ccuggcucuagcagcacagaaauauuggcacagggaagcgagucugccaauauuggcugugcugcuccagg

SEQ ID NO: 328 - RNA, 118 nt, Homo sapiens
ccugggggaggcacuggcaggccugcccgacacucuuucccuguugcacuacuauaggccgcugggaagcagugcaaugaugaaagggcaucggucagguccagccugcuacccuggg

SEQ ID NO: 329 - RNA, 84 nt, Homo sapiens
ccuuaaaguacuguagcagcacaucaugguuuacaugcuacagucaagaugcgaaucauuauuugcugcucuagaaauuuaagg

SEQ ID NO: 330 - RNA, 93 nt, Homo sapiens
ccuuccggcgucccaggcggggcgccgcgggaccgcccucgugucuguggcggugggaucccgcggccguguuuuccugguggcccggccaug

SEQ ID NO: 331 - RNA, 83 nt, Homo sapiens
ccuuggaguaaaguagcagcacauaaugguuuguggauuuugaaaaggugcaggccauauugugcugccucaaaaauacaagg

SEQ ID NO: 332 - RNA, 130 nt, Homo sapiens
cgcagugcaaggccgggccuggcggggugagguaguagguugugugguuucagggcagugauguugccccucggaagauaacuauacaaccuacugccuucccugaggagcccagugacacgaccccaug

SEQ ID NO: 333 - RNA, 167 nt, Homo sapiens
cgcccgccuugcguccagccugccgggggcucccaggccggcgcccgagcucuggcuccgugucuucacucccgugcuuguccgaggagggagggagggacgggggcugugcuggggcagcuggaacaacgcaggucgccgggccggcugggcgaguuggccgggcg

SEQ ID NO: 334 - RNA, 110 nt, Homo sapiens
cgccucagagccgcccgccguuccuuuuuccuaugcauauacuucuuugaggaucuggccuaaagagguauagggcaugggaaaacggggcggucggguccuccccagcg

SEQ ID NO: 335 - RNA, 79 nt, Homo sapiens
cgcucccgccccgcgacgagccccucgcacaaaccggaccugagcguuuuguucguucggcucgcgugaggcaggggcg

SEQ ID NO: 336 - RNA, 67 nt, Homo sapiens
cggcugggguuccuggggaugggauuugcuuccugucacaaaucacauugccagggauuuccaaccg

SEQ ID NO: 337 - RNA, 85 nt, Homo sapiens
cggcuguccucuccaacaauauccuggugcugagugaugacucaggcgacuccagcaucagugauuuuguugaagagggcagcug

SEQ ID NO: 338 - RNA, 94 nt, Homo sapiens
cgggcagcgggugccaggcacggugucagcaggcaacauggccgagaggccggggccuccgggcggcgccguguccgcgaccgcguacccugac

SEQ ID NO: 339 - RNA, 75 nt, Homo sapiens
cguucuuaguugguggagcgauuugucugguuaauuccgauaacgaacgagacucuggcaugcuaacuaguuacg

SEQ ID NO: 340 - RNA, 86 nt, Homo sapiens
cguugucuauauauacccuguagaaccgaauuugugugguauccguauagucacagauucgauucuaggggaauauauggucgaug

SEQ ID NO: 341 - RNA, 130 nt, Homo sapiens
cuaguggagguacugugcuuuguguguuggaggaugaaaguacggagugauccaucggcuaagugucuugucacaaugcugacacucaaacugcugacagcacacguuuuucacaguacuuacgccccag

SEQ ID NO: 342 - RNA, 85 nt, Homo sapiens
cucaagcugugacucuccagagggaugcacuuucucuuaugugaaaaaaaagaaggcgcuucccuuuagagcguuacgguuuggg

SEQ ID NO: 343 - RNA, 85 nt, Homo sapiens
cucaggcugugacacucuagagggaagcgcuuucuguugucugaaagaaaggaaagugcauccuuuuagaguguuacuguuugag

SEQ ID NO: 344 - RNA, 85 nt, Homo sapiens
cucaggcugugacccuccagagggaaguacuuucuguugucugagagaaaagaaagugcuucccuuuggacuguuucgguuugag

SEQ ID NO: 345 - RNA, 71 nt, Homo sapiens
cucccuucaacuuuaacauggaagugcuuucugugacuuuaaaaguaagugcuuccauguuuuaguaggag

SEQ ID NO: 346 - RNA, 68 nt, Homo sapiens
cuccggugccuacugagcugauaucaguucucauuuuacacacuggcucaguucagcaggaacaggag

SEQ ID NO: 347 - RNA, 62 nt, Homo sapiens
cucucuccauuacacuacccugccucuucuccaugagaggcagcgggguguaguggauagag

SEQ ID NO: 348 - RNA, 68 nt, Homo sapiens
cucugcguguucacagcggaccuugauuuaaugucuauacaauuaaggcacgcggugaaugccaagag

SEQ ID NO: 349 - RNA, 74 nt, Homo sapiens
cugaagagaugguagacuauggaacguaggcguuaugauuucugaccuauguaacaugguccacuaacucucag

SEQ ID NO: 350 - RNA, 105 nt, Homo sapiens
cugacucccaccccgaguauccuguacugagcugccccgagcugggcagcaugaagggccucggggcagcucaguacaggaugccccagggaggauggagaucag

SEQ ID NO: 351 - RNA, 105 nt, Homo sapiens
cugaugugacagcuucuguagcacuaaagugcuuauagugcagguaguguuuaguuaucuacugcauuaugagcacuuaaaguacugcuagcuguagaacuccag

SEQ ID NO: 352 - RNA, 118 nt, Homo sapiens
cugcccagugcuauuguuugguacuuaaagcgagguugcccuuuguauauucgguuuauugacauggaauauacaagggcaagcucucugugaguaucaaaccuugucuuggacccag

SEQ ID NO: 353 - RNA, 72 nt, Homo sapiens
cugccucucugggccugugucuuaggcucugcaagaucaaccgagcaaagcacacggccugcagagaggcag

SEQ ID NO: 354 - RNA, 89 nt, Homo sapiens
cuggccuccagggcuuuguacaugguaggcuuucauucauucguuugcacauucggugaaggucuacugugugccaggcccugugccag

SEQ ID NO: 355 - RNA, 89 nt, Homo sapiens
cuggcucagguguauucuacagugcacgugucuccaguguggcucggaggcuggagacgcggcccuguuggaguaacaacugaagccgg

SEQ ID NO: 356 - RNA, 80 nt, Homo sapiens
cuguucuguguauggcacugguagaauucacugugaacagucucagucagugaauuaccgaagggccauaaacagagcag

SEQ ID NO: 357 - RNA, 76 nt, Homo sapiens
cuuaccuagaaauuguugccugucugagcgacgcuucaaacucagcuucagcaggucugcagggacaucagguagg

SEQ ID NO: 358 - RNA, 83 nt, Homo sapiens
cuuagcagagcuguggagugugacaaugguguuugugucuaaacuaucaaacgccauuaucacacuaaauagcuacugcuagg

SEQ ID NO: 359 - RNA, 80 nt, Homo sapiens
cuucgcuccccuccgccuucucuucccgguucuucccggagucgggaaaagcuggguugagagggcgaaaaaggaugagg

SEQ ID NO: 360 - RNA, 87 nt, Homo sapiens
cuuguuccacucuagcagcacguaaauauuggcguagugaaauauauauuaaacaccaauauuacugugcugcuuuagugugacagg

SEQ ID NO: 361 - RNA, 66 nt, Homo sapiens
cuuugccgagacuagagucacauccugacacaacucuuguccuggugugcuagaguacucgaagag

SEQ ID NO: 362 - RNA, 66 nt, Homo sapiens
cuuugcuuuaacauggggguaccugcugugugaaacaaaaguaagugcuuccauguuucaguggag

SEQ ID NO: 363 - RNA, 87 nt, Homo sapiens
gaaagcgcuuuggaaugacacgaucacucccguugagugggcacccgagaagccaucgggaaugucguguccgcccagugcucuuuc

SEQ ID NO: 364 - RNA, 107 nt, Homo sapiens
gaacauccaggucuggggcaugaaccuggcauacaauguagauuucuguguucguuaggcaacagcuacauugucugcuggguuucaggcuaccuggaaacauguuc

SEQ ID NO: 365 - RNA, 95 nt, Homo sapiens
gaagaucucaggcagugacccucuagauggaagcacugucuguugucuaagaaaagaucgugcauccuuuuagaguguuacuguuugagaaaauc

SEQ ID NO: 366 - RNA, 115 nt, Homo sapiens
gaaguagauugaagccaguugauuagggugcuuagcuguuaacuaaguguuuguggguuuaagucccauuggucuaguaagggcuuagcuuaauuaaaguggcugauuugcguuc

SEQ ID NO: 367 - RNA, 90 nt, Homo sapiens
gacacagugugagaccucgggcuacaacacaggacccgggcgcugcucugaccccucgugucuuguguugcagccggagggacgcagguc

SEQ ID NO: 368 - RNA, 81 nt, Homo sapiens
gacuaugccuccccgcauccccuagggcauugguguaaagcuggagacccacugccccaggugcugcuggggguuguaguc

SEQ ID NO: 369 - RNA, 65 nt, Homo sapiens
gagcaucuuaccggacagugcuggauuucccagcuugacucuaacacugucugguaacgauguuc

SEQ ID NO: 370 - RNA, 134 nt, Homo sapiens
gaggaagccggcggaguucugguaucguugcugcagcugguguugugaaucaggccgacgagcagcgcauccucuuacccggcuauuucacgacaccaggguugcaucauacccauccucuccaggcgagccuc

SEQ ID NO: 371 - RNA, 84 nt, Homo sapiens
gaggacuccaaggaacauucaacgcugucggugaguuugggauuugaaaaaaccacugaccguugacuguaccuugggguccuu

SEQ ID NO: 372 - RNA, 198 nt, Homo sapiens
gaggcugcgaggagugagcggcuuguaugggaccaugcagccagagggugacagagccacccagggcuccugacuccagguccuguguguuaccuagaaauagcacuggacuuggagucagaaggccugaguggagucaccuuccccacucucuggcugggugaccccggagcaagccauuugaacucuccgagccuc

SEQ ID NO: 373 - RNA, 68 nt, Homo sapiens
gagguccucaaaacugaggggcauuuucugugguuugaaaggaaagugcacccaguuuuggggauguc

SEQ ID NO: 374 - RNA, 92 nt, Homo sapiens
gagugcacagggcucugaccuaugaauugacagccagugcucucgucuccccucuggcugccaauuccauaggucacagguauguucgccuc

SEQ ID NO: 375 - RNA, 81 nt, Homo sapiens
gauacuaaacuguuuuugcgauguguuccuaauaugcacuauaaauauauugggaacauuuugcauguauaguuuuguauc

SEQ ID NO: 376 - RNA, 81 nt, Homo sapiens
gauacucgaaggagagguuguccguguugucuucucuuuauuuaugaugaaacauacacgggaaaccucuuuuuuaguauc

SEQ ID NO: 377 - RNA, 119 nt, Homo sapiens
gaucccccuucauugacuccgaggggauacagcagcaauucauguuuugaaguguucuaaaugguucaaaacgugaggcgcugcuauacccccucguggggaagguagaaggugggguc

SEQ ID NO: 378 - RNA, 69 nt, Homo sapiens
gaugggcgucuuaccagacaugguuagaccuggcccucugucuaauacugucugguaaaaccguccauc

SEQ ID NO: 379 - RNA, 82 nt, Homo sapiens
gcaagaaaaaugagggacuuucaggggcagcuguguuuucugacucagucauaaugccccuaaaaauccuuauuguucuugc

SEQ ID NO: 380 - RNA, 134 nt, Homo sapiens
gcacucccaaggaguuugguuuuguuuggguuuguucuagguauggucccagggaucccagaucaaaccaggccccugggccuauccuagaaccaaccuaagcucgcgcaucauuccuggaacaucaagagugu

SEQ ID NO: 381 - RNA, 120 nt, Homo sapiens
gcagaagaaugaacugcuccuucucccauacccauugcauaucggaguugugaauucucaaaacaccuccugugugcauggauuacaggagggugagccuugucaucgugaaucuucugc

SEQ ID NO: 382 - RNA, 83 nt, Homo sapiens
gcagggaaaaugagggacuuuugggggcagauguguuuccauuccacuaucauaaugccccuaaaaauccuuauugcucuugc

SEQ ID NO: 383 - RNA, 78 nt, Homo sapiens
gcagugggaaggggggccgauacacuguacgagagugaguagcaggucucacagugaaccggucucuuucccuacugu

SEQ ID NO: 384 - RNA, 144 nt, Homo sapiens
gcagugggucuuauacuuuccggaucaagauuagaggcucugcucuccguguucacagcggaccuugauuuaaugucauacaauuaaggcacgcggugaaugccaagagcggagccuacggcugcacuugaaggacaccaaagc

SEQ ID NO: 385 - RNA, 111 nt, Homo sapiens
gcauccucaggaccugggcuugggugguaggaggaauuggugcuggucuuucauuuuggauuugacuccagccccacagccucagccaccccagccaauugucauaggagc

SEQ ID NO: 386 - RNA, 94 nt, Homo sapiens
gccaaaugcuuugcuagagcugguaaaauggaaccaaaucgacuguccaauggauuugguccccuucaaccagcuguagcugugcauugauggc

SEQ ID NO: 387 - RNA, 84 nt, Homo sapiens
gccagucucuaggucccugagacccuuuaaccugugaggacauccagggucacaggugagguucuugggagccuggcgucuggc

SEQ ID NO: 388 - RNA, 82 nt, Homo sapiens
gccaguguugagaggcggagacuugggcaauugcuggacgcugcccugggcauugcacuugucucggucugacagugccggc

SEQ ID NO: 389 - RNA, 111 nt, Homo sapiens
gcccacaaugugaggacacgagagaagcacuggacuuagggucagaaggccugagucucucugcugcagaugggcucucugucccugagccaagcuuuguccucccugggc

SEQ ID NO: 390 - RNA, 136 nt, Homo sapiens
gccccaaacccuccuugggaagugaagcucaggcugugauuucaagccagggggcguuuuucuauaacuggaugaaaagcaccuccagagcuugaagcucacaguuugagagcaaucgucuaaggaaguugauggc

SEQ ID NO: 391 - RNA, 76 nt, Homo sapiens
gcccucggaggacuccauuuguuuugaugauggauucuuaugcuccaucaucgucucaaaugagucuucagagggu

SEQ ID NO: 392 - RNA, 86 nt, Homo sapiens
gcccugccggggcuaaagugcugacagugcagauagugguccucuccgugcuaccgcacuguggguacuugcugcuccagcagggc

SEQ ID NO: 393 - RNA, 90 nt, Homo sapiens
gcccuuuggggugagguaguagguuguauaguuuggggcucugcccugcuaugggauaacuauacaaucuacugucuuuccugaaguggc

SEQ ID NO: 394 - RNA, 78 nt, Homo sapiens
gccggagaaagccgccgccgccggccccaccccagcaccagcgccccggccggggggaggcgggggcagccaccuggc

SEQ ID NO: 395 - RNA, 104 nt, Homo sapiens
gccugauuccaggcugagguaguaguuuguacaguuugagggucuaugauaccacccgguacaggagauaacuguacaggccacugccuugccaggaacagcgc

SEQ ID NO: 396 - RNA, 152 nt, Homo sapiens
gccuggugcuagucacuggggcaaagaugacuaaaacacuuuuccugcccucgaggagcucacagucuaguaugucucauccccuacuagacugaagcuccuugaggacagggauggucauacucaccucgguguugcccaaaccuccaggc

SEQ ID NO: 397 - RNA, 82 nt, Homo sapiens
gccuguugccacaaacccguagauccgaacuugugguauuaguccgcacaagcuuguaucuauagguaugugucuguuaggc

SEQ ID NO: 398 - RNA, 84 nt, Homo sapiens
gccuuggaaaugacagccuuggagucugugcaguccuuucacaggcacgaucucacgcaugaccuccaaggaaggcagcagggc

SEQ ID NO: 399 - RNA, 83 nt, Homo sapiens
gccuuggccauguaaaagugcuuacagugcagguagcuuuuugagaucuacugcaauguaagcacuucuuacauuaccauggu

SEQ ID NO: 400 - RNA, 101 nt, Homo sapiens
gcgagaagaucucaugcugugacucucuggagggaagcacuuucuguugucugaaagaaaacaaagcgcuucucuuuagaguguuacgguuugagaaaagc

SEQ ID NO: 401 - RNA, 88 nt, Homo sapiens
gcgagauuuucuguugugcuugaucuaaccaugugguugcgagguaugaguaaaacaugguuccgucaagcaccauggaacgucacgc

SEQ ID NO: 402 - RNA, 118 nt, Homo sapiens
gcgcccugugugugucucucucuguguccugccagugguuuuacccuaugguagguuacgucaugcuguucuaccacaggguagaaccacggacaggauaccggggcacccucugcgu

SEQ ID NO: 403 - RNA, 106 nt, Homo sapiens
gcgggugcccggugcucgguuuguaggcagugucauuagcugauuguacuguggugguuacaaucacuaacuccacugccaucaaaacaaggcacagcaucaccgc

SEQ ID NO: 404 - RNA, 85 nt, Homo sapiens
gcgguggccagugucauuuuugugauguugcagcuaguaauaugagcccaguugcauagucacaaaagugaucauuggaaacugu

SEQ ID NO: 405 - RNA, 85 nt, Homo sapiens
gcuaagcacuuacaacuguuugcagaggaaacugagacuuuguaacuaugucucagucucaucugcaaagaaguaagugcuuugc

SEQ ID NO: 406 - RNA, 87 nt, Homo sapiens
gcucauacugggauacucaaaaugggggcgcuuuccuuuuugucuguacugggaagugcuucgauuuuggggugucccuguuugagu

SEQ ID NO: 407 - RNA, 84 nt, Homo sapiens
gcucccccucucuaauccuugcuaccugggugagagugcugucugaaugcaaugcaccugggcaaggauucugagagcgagagc

SEQ ID NO: 408 - RNA, 84 nt, Homo sapiens
gcucccccucuugaauccuuggaaccuaggugugagugcuauuucagugcaacacaccuauucaaggauucaaagaggcugagc

SEQ ID NO: 409 - RNA, 124 nt, Homo sapiens
gcucccucgccccacccugcccgggacccaguucaaguaauucaggauagguugugugcuguccagccuguucuccauuacuuggcucggggaccggugcccugcagccuuggggugagggggc

SEQ ID NO: 410 - RNA, 79 nt, Homo sapiens
gcuccgucgccccaguguucagacuaccuguucaggacaaugccguuguacaguagucugcacauugguuagacugggc

SEQ ID NO: 411 - RNA, 89 nt, Homo sapiens
gcucgggcagccguggccaucuuacugggcagcauuggauggagucaggucucuaauacugccugguaaugaugacggcggagcccugc

SEQ ID NO: 412 - RNA, 100 nt, Homo sapiens
gcucgguugccgugguugcgggcccugcccgcccgccagcucgcugacagcacgacucagggcggagggaaguagguccguuggucggucgggaacgagg

SEQ ID NO: 413 - RNA, 116 nt, Homo sapiens
gcucuggauaccugugugugaugagcuggcaguguauuguuagcugguugaauaugugaauggcaucggcuaacaugcaacugcugucuuauugcauauacaaugaacaucagagu

SEQ ID NO: 414 - RNA, 84 nt, Homo sapiens
gcucuuccucucuaauccuuugucccugggugagagugcuuucugaaugcaaugcacccgggcaaggauucugagagggugagc

SEQ ID NO: 415 - RNA, 132 nt, Homo sapiens
gcucuuggccuggccuccugcagugccacgcuccguguauuugacaagcugaguuggacacuccaugugguagagugucaguuugucaaauaccccaagugcggcacaugcuuaccagcucuaggccagggc

SEQ ID NO: 416 - RNA, 117 nt, Homo sapiens
gcugccuggccucccugggcucugccucccgugccuacugagcugaaacacaguugguuuguguacacuggcucaguucagcaggaacaggggucaagcccccuuggagccugcagc

SEQ ID NO: 417 - RNA, 87 nt, Homo sapiens
gcugcuggccagagcucuuuucacauugugcuacugucugcaccugucacuagcagugcaauguuaaaagggcauuggccguguagu

SEQ ID NO: 418 - RNA, 83 nt, Homo sapiens
gcugcuguugggagacccuggucugcacucuaucuguauucuuacugaagggagugcagggcaggguuucccauacagagggc

SEQ ID NO: 419 - RNA, 83 nt, Homo sapiens
gcuggcgacgggacauuauuacuuuugguacgcgcugugacacuucaaacucguaccgugaguaauaaugcgccguccacggc

SEQ ID NO: 420 - RNA, 76 nt, Homo sapiens
gcuggguccagugguucuuaacaguucaacaguucuguagcgcaauugugaaauguuuaggaccacuagacccggc

SEQ ID NO: 421 - RNA, 95 nt, Homo sapiens
gcuguugaggcugcgcagccaggcccugacggugggguggcugcgggccuucugaaggucucccacguuguggcccagcagcgcagucacguugc

SEQ ID NO: 422 - RNA, 84 nt, Homo sapiens
gcuucccgaggccacaugcuucuuuauauccccauauggauuacuuugcuauggaauguaaggaagugugugguuucggcaagu

SEQ ID NO: 423 - RNA, 117 nt, Homo sapiens
gcuucuguugcagaaccugagugugcaucguggucaaaugcucagacuccugugguggcugcucaugcaccacggauguuugagcaugugcuacggugucuacuuuugcuacauugc

SEQ ID NO: 424 - RNA, 77 nt, Homo sapiens
gcuugggaaacauacuucuuuauaugcccauauggaccugcuaagcuauggaauguaaagaaguauguaucucaggc

SEQ ID NO: 425 - RNA, 78 nt, Homo sapiens
gcuuuccuuugugcuugaucuaaccaugugguggaacgauggaaacggaacaugguucugucaagcaccgcggaaagc

SEQ ID NO: 426 - RNA, 85 nt, Homo sapiens
gcuuuuuguucuaaggugcaucuagugcagauagugaaguagauuagcaucuacugcccuaagugcuccuucuggcauaagaagu

SEQ ID NO: 427 - RNA, 147 nt, Homo sapiens
ggaguuugaagcaacauuggaagcugugugcauccggguugagguaguagguuguaugguuuagaguuacacccugggaguuaacuguacaaccuucuagcuuuccuuggagcacacuugagccgucgaggaauucuucaucacuuu

SEQ ID NO: 428 - RNA, 127 nt, Homo sapiens
ggaugccacauucagccauucagugugcagugccuuucacagggaggugucauuuaugugaacuaaaauauaaauuucaccuuucugagaaggguaauguacagcaugcacugcauauguggugucc

SEQ ID NO: 429 - RNA, 55 nt, Homo sapiens
ggcaagaugcuggcauagcuguugaacugggaaccugcuaugccaacauauugcc

SEQ ID NO: 430 - RNA, 85 nt, Homo sapiens
ggcacuugggaauggcaaggaaaccguuaccauuacugaguuuaguaaugguaaugguucucuugcuauacccagaaaacgugcc

SEQ ID NO: 431 - RNA, 90 nt, Homo sapiens
ggcagucuuugcuacuguaaacauccuugacuggaagcuguaagguguucagaggagcuuucagucggauguuuacagcggcaggcugcc

SEQ ID NO: 432 - RNA, 83 nt, Homo sapiens
ggccccgccaacccaguguucagacuaccuguucaggaggcucucaauguguacaguagucugcacauugguuaggcugggcu

SEQ ID NO: 433 - RNA, 62 nt, Homo sapiens
ggccucaaauguggagcacuauucugauguccaaguggaaagugcugcgacauuugagcguc

SEQ ID NO: 434 - RNA, 83 nt, Homo sapiens
ggccucucucuccguguucacagcggaccuugauuuaaauguccauacaauuaaggcacgcggugaaugccaagaauggggcu

SEQ ID NO: 435 - RNA, 76 nt, Homo sapiens
ggccucugugugauauguuugauauauuagguuguuauuuaauccaacuauauaucaaacauauuccuacaguguc

SEQ ID NO: 436 - RNA, 63 nt, Homo sapiens
ggcggcggcggcggcggcgacucugcaggcggaguuucgcggcggcggcaccaggguuacgcc

SEQ ID NO: 437 - RNA, 87 nt, Homo sapiens
ggcgggcagcuguggugcauuguaguugcauugcauguucuggugguacccaugcaauguuuccacagugcaucacagaggccugcc

SEQ ID NO: 438 - RNA, 81 nt, Homo sapiens
ggcugcccuggcucaguuaucacagugcugaugcugucuauucuaaagguacaguacugugauaacugaaggauggcagcc

SEQ ID NO: 439 - RNA, 104 nt, Homo sapiens
ggcuggacagaguugucaugugucugccugucuacacuugcugugcagaacauccgcucaccuguacagcaggcacagacaggcagucacaugacaacccagcc

SEQ ID NO: 440 - RNA, 90 nt, Homo sapiens
ggcuggacagcgggcaacggaaucccaaaagcagcuguugucuccagagcauuccagcugcgcuuggauuucguccccugcucuccugcc

SEQ ID NO: 441 - RNA, 84 nt, Homo sapiens
ggcuguggcuggauucaaguaauccaggauaggcuguuuccaucugugaggccuauucuugauuacuuguuucuggaggcagcu

SEQ ID NO: 442 - RNA, 79 nt, Homo sapiens
gggagcugagggcugggucuuugcgggcgagaugagggugucggaucaacuggccuacaaagucccaguucucggcccc

SEQ ID NO: 443 - RNA, 129 nt, Homo sapiens
gggaugccacauucagccauucagcguacagugccuuucacagggaggugucauuuaugugaacuaaaauauaaauuucaccuuucugagaaggguaauguacagcaugcacugcauaugugguguccc

SEQ ID NO: 444 - RNA, 88 nt, Homo sapiens
gggauuugggagcuuaucagaaucuccagggguacuuuauaauuucaaaaagucccccaggugugauucugauuugcuuccuuuuccu

SEQ ID NO: 445 - RNA, 66 nt, Homo sapiens
gggcaaccguggcuuucgauuguuacugugggaacuggagguaacagucuacagccauggucgccc

SEQ ID NO: 446 - RNA, 86 nt, Homo sapiens
gggcgggggcccucgucuuacccagcaguguuugggugcgguugggagucucuaauacugccggguaaugauggaggccccugucc

SEQ ID NO: 447 - RNA, 200 nt, Homo sapiens
ggggagaucaauaaaugagugguuaauuaauuuuauuaacgaguugguuuugcguguuggggucauuaguguucuuguaguugaaauacaacgaugguuuuucauaucauuggucgugguuguaguccgugcgagaauaaugacauaugcuuuauuuuuguuaagugugguuuuaguaaugggguuugugggguuuucuu

SEQ ID NO: 448 - RNA, 82 nt, Homo sapiens
ggggcggccgcucucccuguccuccaggagcucacgugugccugccugugagcgccucgacgacagagccggcgccugcccc

SEQ ID NO: 449 - RNA, 89 nt, Homo sapiens
ggggcuucgccacugauuguccaaacgcaauucuuguacgagucugcggccaaccgagaauuguggcuggacaucuguggcugagcucc

SEQ ID NO: 450 - RNA, 80 nt, Homo sapiens
gggggcgagggauuggagagaaaggcaguuccugaugguccccuccccaggggcuggcuuuccucugguccuucccuccc

SEQ ID NO: 451 - RNA, 81 nt, Homo sapiens
ggguuugggggaacauucaaccugucggugaguuugggcagcucaggcaaaccaucgaccguugaguggacccugaggccu

SEQ ID NO: 452 - RNA, 93 nt, Homo sapiens
gguaacugcccucaaggagcuuacaaucuagcuggggguaaaugacuugcacaugaacacaacuagacugugagcuucuagagggcagggacc

SEQ ID NO: 453 - RNA, 80 nt, Homo sapiens
gguaccugagaagagguugucugugaugaguucgcuuuuauuaaugacgaauauaacacagauggccuguuuucaguacc

SEQ ID NO: 454 - RNA, 98 nt, Homo sapiens
gguacuucucagucuguggcacucagccuugagggcacuuucuggugccagaaugaaagugcugucauagcugagguccaaugacugaggcgagcacc

SEQ ID NO: 455 - RNA, 79 nt, Homo sapiens
gguacuuggagagaggugguccguggcgcguucgcuuuauuuauggcgcacauuacacggucgaccucuuugcaguauc

SEQ ID NO: 456 - RNA, 79 nt, Homo sapiens
ggucacaaucaacauucauugcugucgguggguugaacuguguggacaagcucacugaacaaugaaugcaacuguggcc

SEQ ID NO: 457 - RNA, 86 nt, Homo sapiens
gguccuugcccucaaggagcucacagucuauugaguuaccuuucugacuuucccacuagauugugagcuccuggagggcaggcacu

SEQ ID NO: 458 - RNA, 84 nt, Homo sapiens
ggucuuuugaggcaaaguucugagacacuccgacucugaguaugauagaagucagugcacuacagaacuuugucucuagaggcu

SEQ ID NO: 459 - RNA, 95 nt, Homo sapiens
ggugagugcguuuccaagugugaagggacccuuccuguagugucuuauauacaauacaguaggaauguuccuucuuugccacucauacaccuuua

SEQ ID NO: 460 - RNA, 64 nt, Homo sapiens
gguugcaaagcugaaacuuaaaggaauugguggaagggcaccaccaggaguggagccugcggcu

SEQ ID NO: 461 - RNA, 87 nt, Homo sapiens
gguugcuucagugaacauucaacgcugucggugaguuuggaauuaaaaucaaaaccaucgaccguugauuguacccuauggcuaacc

SEQ ID NO: 462 - RNA, 90 nt, Homo sapiens
guaccaccuugucggguagcuuaucagacugauguugacuguugaaucucauggcaacaccagucgaugggcugucugacauuuugguau

SEQ ID NO: 463 - RNA, 89 nt, Homo sapiens
guaccuggugugauuauaaagcaaugagacugauugucauaugucguuugugggauccgucucaguuacuuuauagccauaccugguau

SEQ ID NO: 464 - RNA, 108 nt, Homo sapiens
guagauugaagccaguugauuagggugcuuagcuguuaacuaaguguuuguggguuuaagucccauuggucuaguaagggcuuagcuuaauuaaaguggcugauuugc

SEQ ID NO: 465 - RNA, 62 nt, Homo sapiens
guaggcaguguauuguuagcuggcugcuugggucaagucagcagccacaacuacccugccac

SEQ ID NO: 466 - RNA, 80 nt, Homo sapiens
guauccucagcuuugagaacugaauuccauggguugugucagugucagaccucugaaauucaguucuucagcugggauau

SEQ ID NO: 467 - RNA, 84 nt, Homo sapiens
gucagaauaaugucaaagugcuuacagugcagguagugauaugugcaucuacugcagugaaggcacuuguagcauuauggugac

SEQ ID NO: 468 - RNA, 89 nt, Homo sapiens
gucagcagugccuuagcagcacguaaauauuggcguuaagauucuaaaauuaucuccaguauuaacugugcugcugaaguaagguugac

SEQ ID NO: 469 - RNA, 79 nt, Homo sapiens
guccagggugugugacugguugaccagaggggcaugcacuguguucacccugugggccaccuagucaccaacccucagc

SEQ ID NO: 470 - RNA, 85 nt, Homo sapiens
gucccccccggcccagguucugugauacacuccgacucgggcucuggagcagucagugcaugacagaacuugggcccggaaggac

SEQ ID NO: 471 - RNA, 86 nt, Homo sapiens
guccugggggcuccaaagugcuguucgugcagguagugugauuacccaaccuacugcugagcuagcacuucccgagcccccgggac

SEQ ID NO: 472 - RNA, 104 nt, Homo sapiens
gucggccggcccuggguccaucuuccaguacaguguuggauggucuaauugugaagcuccuaacacugucugguaaagauggcucccggguggguucucucggc

SEQ ID NO: 473 - RNA, 69 nt, Homo sapiens
gucuacccaguguuuagacuaucuguucaggacucccaaauuguacaguagucugcacauugguuaggc

SEQ ID NO: 474 - RNA, 83 nt, Homo sapiens
gucuagugcuguguggaagacuagugauuuuguuguucugauguacuacgacaacaagucacagccggccucauagcgcagac

SEQ ID NO: 475 - RNA, 147 nt, Homo sapiens
gucugucugucgggucuguccaccugccgcgccccccgggcugagguaggagguuguauaguugaggaggacacccaaggagaucacuauacggccuccuagcuuuccccaggcugcgcccugcacgggacggggcccggcggggac

SEQ ID NO: 476 - RNA, 85 nt, Homo sapiens
gucuucuguauauacccuguagauccgaauuuguguaaggaauuuuguggucacaaauucguaucuaggggaauauguaguugac

SEQ ID NO: 477 - RNA, 105 nt, Homo sapiens
gugaaacugggcucaaggugaggggugcuaucugugauugagggacaugguuaauggaauugucucacacagaaaucgcacccgucaccuuggccuacuuaucac

SEQ ID NO: 478 - RNA, 67 nt, Homo sapiens
gugacccucuagauggaagcacugucuguugucuaagaaaagaucgugcaucccuuuagaguguuac

SEQ ID NO: 479 - RNA, 100 nt, Homo sapiens
gugagcgggcgcggcagggaucgcgggcggguggcggccuagggcgcggagggcggaccgggaauggcgcgccgugcgccgccggcguaacugcggcgcu

SEQ ID NO: 480 - RNA, 90 nt, Homo sapiens
gugaguguuucuuuggcagugucuuagcugguuguugugagcaauaguaaggaagcaaucagcaaguauacugcccuagaagugcugcac

SEQ ID NO: 481 - RNA, 95 nt, Homo sapiens
gugccuggccugaggagcagggcuuagcugcuugugagcaggguccacaccaagucguguucacaguggcuaaguuccgccccccaggcccucac

SEQ ID NO: 482 - RNA, 96 nt, Homo sapiens
gugcucugugggaugagguaguagauuguauaguuuuagggucauaccccaucuuggagauaacuauacagucuacugucuuucccacggugguac

SEQ ID NO: 483 - RNA, 74 nt, Homo sapiens
gugcuuucagcuucuuuacagugcugccuuguagcauucaggucaagcagcauuguacagggcuaugaaagaac

SEQ ID NO: 484 - RNA, 98 nt, Homo sapiens
guggaccggcuggccccaucuggaagacuagugauuuuguuguugucuuacugcgcucaacaacaaaucccagucuaccuaauggugccagccaucgc

SEQ ID NO: 485 - RNA, 84 nt, Homo sapiens
guggcagcuugguggucguaugugugacgccauuuacuugaaccuuuaggagugacaucacauauacggcagcuaaacugcuac

SEQ ID NO: 486 - RNA, 71 nt, Homo sapiens
guggccuauggcuuuucauuccuaugugauugcugucccaaacucauguagggcuaaaagccaugggcuac

SEQ ID NO: 487 - RNA, 77 nt, Homo sapiens
guggccucguucaaguaauccaggauaggcugugcaggucccaaugggccuauucuugguuacuugcacggggacgc

SEQ ID NO: 488 - RNA, 83 nt, Homo sapiens
guggucucagaaucgggguuuugagggcgagaugaguuuauguuuuauccaacuggcccucaaagucccgcuuuuggggucau

SEQ ID NO: 489 - RNA, 112 nt, Homo sapiens
guguuccuuuccuacuuauuacugcuaacgaaugcucugacuuuauugcacuacuguacuuuacagcuagcagugcaauaguauugucaaagcaucugaaagcaggaugcac

SEQ ID NO: 490 - RNA, 86 nt, Homo sapiens
guuaaaucaggaauuuuaaacaauuccuagacaauauguauaauguucauaagucauuccuagaaauuguucauaaugccuguaac

SEQ ID NO: 491 - RNA, 70 nt, Homo sapiens
guuggauucggggccguagcacugucugagagguuuacauuucucacagugaaccggucucuuuuucagc

SEQ ID NO: 492 - RNA, 85 nt, Homo sapiens
guuuugagcgggggucaagagcaauaacgaaaaauguuugucauaaaccguuuuucauuauugcuccugaccuccucucauuugc

SEQ ID NO: 493 - RNA, 89 nt, Homo sapiens
guuuugcuguugucggguggaucacgaugcaauuuugaugaguaucauaggagaaaaauugcacgguauccaucuguaaaccgcaggac

SEQ ID NO: 494 - RNA, 60 nt, Homo sapiens
uaaucucagcuggcaacugugagauguucauacaaucccucacaguggucucugggauua

SEQ ID NO: 495 - RNA, 78 nt, Homo sapiens
uacaauuaguuuugcagguuugcauuucagcguauauauguauauguggcugugcaaauccaugcaaaacugauugug

SEQ ID NO: 496 - RNA, 72 nt, Homo sapiens
uacaucggccauuauaauacaaccugauaaguguuauagcacuuaucagauuguauuguaauugucugugua

SEQ ID NO: 497 - RNA, 185 nt, Homo sapiens
uacauuuucccggauggaagcccgaaguuugagggagaaacuugugaggaaauaaaggaaguuaggcugaggggcagagagcgagacuuuucuauuuuccaaaagcucggucugaggccccucagucuugcuuccuaacccgcgcuugaguuucuccccgcuuggaugcucucaggggcagugug

SEQ ID NO: 498 - RNA, 55 nt, Homo sapiens
uaggcaguguaguuagcugauugcuaauaguaccaaucacuaaccacacggccag

SEQ ID NO: 499 - RNA, 89 nt, Homo sapiens
uaggcuguaugcuguuaaugcuaaucgugauagggguuuuugccuccaacugacuccuacauauuagcauuaacaguguaugaugccug

SEQ ID NO: 500 - RNA, 61 nt, Homo sapiens
ucaggcucaguccccucccgauaaaccccuaaauagggacuuucccggggggugacccugg

SEQ ID NO: 501 - RNA, 83 nt, Homo sapiens
ucaggcugugacccucuugagggaagcacuuucuguugucugaaagaagagaaagugcuuccuuuuagaggcuuacugucuga

SEQ ID NO: 502 - RNA, 64 nt, Homo sapiens
ucaucgaggcuagagucacgcuuggguaucggcuauugccugagugugcuagaguccucgaaga

SEQ ID NO: 503 - RNA, 83 nt, Homo sapiens
ucaugcuguggcccuccagagggaagcgcuuucuguugucugaaagaaaacaaagcgcuccccuuuagagguuuacgguuuga

SEQ ID NO: 504 - RNA, 87 nt, Homo sapiens
uccaugugcuucucuuguccuucauuccaccggagucugucucauacccaaccagauuucaguggagugaaguucaggaggcaugga

SEQ ID NO: 505 - RNA, 87 nt, Homo sapiens
ucccaugcugugacccucuagagggaagcacuuucuguugucugaaagaaaccaaagcgcuucccuuuggagcguuacgguuugaga

SEQ ID NO: 506 - RNA, 88 nt, Homo sapiens
ucccaugcugugacccucuagagggaagcgcuuucuguugucugaaagaaaagaaagugcauccuuuuagagguuuacuguuugagga

SEQ ID NO: 507 - RNA, 82 nt, Homo sapiens
uccccauggcccugucucccaacccuuguaccagugcugggcucagacccugguacaggccugggggacagggaccugggga

SEQ ID NO: 508 - RNA, 102 nt, Homo sapiens
uccucccagcggugccuccaccucacgccccagugcugccgggcccaauuucugauaaaggcagugauccuggggaggaaaugggcagcgauuuggggagga

SEQ ID NO: 509 - RNA, 78 nt, Homo sapiens
uccuggcacccacccguagaaccgaccuugcggggccuucgccgcacacaagcucgugucuguggguccgugucgggg

SEQ ID NO: 510 - RNA, 81 nt, Homo sapiens
uccugugacgggcgagcuuuuggcccggguuauaccugaugcucacguauaagacgagcaaaaagcuuguuggucagagga

SEQ ID NO: 511 - RNA, 85 nt, Homo sapiens
ucucaagcugugacugcaaagggaagcccuuucuguugucuaaaagaaaagaaagugcuucccuuuggugaauuacgguuugaga

SEQ ID NO: 512 - RNA, 85 nt, Homo sapiens
ucucaagcugugacugcaaagggaagcccuuucuguugucugaaagaagagaaagcgcuucccuuugcuggauuacgguuugaga

SEQ ID NO: 513 - RNA, 87 nt, Homo sapiens
ucucaagcugugagucuacaaagggaagcccuuucuguugucuaaaagaaaagaaagugcuucucuuugguggguuacgguuugaga

SEQ ID NO: 514 - RNA, 87 nt, Homo sapiens
ucucaagcugugggucugcaaagggaagcccuuucuguugucuaaaagaagagaaagcgcuucccuuugcuggauuacgguuugaga

SEQ ID NO: 515 - RNA, 87 nt, Homo sapiens
ucucagccugugacccucuagagggaagcgcuuucuguugucugaaagaaaagaaagugcaucuuuuuagaggauuacaguuugaga

SEQ ID NO: 516 - RNA, 87 nt, Homo sapiens
ucucaggcagugacccucuagauggaagcacugucuguuguauaaaagaaaagaucgugcaucccuuuagaguguuacuguuugaga

SEQ ID NO: 517 - RNA, 87 nt, Homo sapiens
ucucaggcugucguccucuagagggaagcacuuucuguugucugaaagaaaagaaagugcuuccuuuuagaggguuaccguuugaga

SEQ ID NO: 518 - RNA, 90 nt, Homo sapiens
ucucaggcugugaccaucuggagguaagaagcacuuucuguuuugugaaagaaaagaaagugcuuccuuucagaggguuacucuuugaga

SEQ ID NO: 519 - RNA, 87 nt, Homo sapiens
ucucaggcugugacccucuaaagggaagcgcuuucuguggucagaaagaaaagcaagugcuuccuuuuagaggguuaccguuuggga

SEQ ID NO: 520 - RNA, 88 nt, Homo sapiens
ucucaggcugugacccucuagagggaagcgcuuucuguuggcuaaaagaaaagaaagcgcuucccuucagaguguuaacgcuuugaga

SEQ ID NO: 521 - RNA, 87 nt, Homo sapiens
ucucaggcugugucccucuagagggaagcgcuuucuguugucugaaagaaaagaaaaugguucccuuuagaguguuacgcuuugaga

SEQ ID NO: 522 - RNA, 83 nt, Homo sapiens
ucucaugcagucauucuccaaaagaaagcacuuucuguugucugaaagcagagugccuucuuuuggagcguuacuguuugaga

SEQ ID NO: 523 - RNA, 84 nt, Homo sapiens
ucucaugcagucauucuccaaaagggagcacuuucuguuugaaagaaaacaaagugccuccuuuuagaguguuacuguuugaga

SEQ ID NO: 524 - RNA, 87 nt, Homo sapiens
ucucaugcugugacccuacaaagggaagcacuuucucuuguccaaaggaaaagaaggcgcuucccuuuggaguguuacgguuugaga

SEQ ID NO: 525 - RNA, 87 nt, Homo sapiens
ucucaugcugugacccucuagagggaagcacuuucucuugucuaaaagaaaagaaagcgcuucucuuuagaggauuacucuuugaga

SEQ ID NO: 526 - RNA, 87 nt, Homo sapiens
ucucaugcugugacccucuagagggaagcgcuuucuguugucugaaagaaaagaacgcgcuucccuauagaggguuacccuuugaga

SEQ ID NO: 527 - RNA, 79 nt, Homo sapiens
ucucccagccugaggugcagugcugcaucucuggucaguugggagucugagaugaagcacuguagcucaggaagagaga

SEQ ID NO: 528 - RNA, 86 nt, Homo sapiens
ucucuuacacaggcugaccgauuucuccugguguucagagucuguuuuugucuagcaccauuugaaaucgguuaugauguaggggg

SEQ ID NO: 529 - RNA, 84 nt, Homo sapiens
ucuuacugcccucggcuucuuuacagugcugccuuguugcauauggaucaagcagcauuguacagggcuaugaaggcauugaga

SEQ ID NO: 530 - RNA, 90 nt, Homo sapiens
ucuucuucuggaagcugguuucacaugguggcuuagauuuuuccaucuuuguaucuagcaccauuugaaaucaguguuuuaggaguaaga

SEQ ID NO: 531 - RNA, 94 nt, Homo sapiens
ugacuccuccaggucuuggaguaggucauuggguggauccucuauuuccuuacgugggccacuggauggcuccuccaugucuuggaguagauca

SEQ ID NO: 532 - RNA, 73 nt, Homo sapiens
ugaggguaugugccuuuggacuacaucguggaagccagcaccaugcaguccaugggcauauacacuugccuca

SEQ ID NO: 533 - RNA, 87 nt, Homo sapiens
ugcccccugcucuggcuggucaaacggaaccaaguccgucuuccugagagguuugguccccuucaaccagcuacagcagggcuggca

SEQ ID NO: 534 - RNA, 71 nt, Homo sapiens
ugcccuagcagcgggaacaguucugcagugagcgaucggugcucugggguauuguuuccgcugccagggua

SEQ ID NO: 535 - RNA, 128 nt, Homo sapiens
uggaggccuugcugguuuggaaaguucauuguucgacaccauggaucuccaggugggucaaguuuagagaugcaccaaccuggaggacuccaugcuguugagcuguucacaagcagcggacacuucca

SEQ ID NO: 536 - RNA, 106 nt, Homo sapiens
uggauguuggccuaguucuguguggaagacuagugauuuuguuguuuuuagauaacuaaaucgacaacaaaucacagucugccauauggcacaggccaugccucua

SEQ ID NO: 537 - RNA, 65 nt, Homo sapiens
uggcacucaaacugugggggcacuuucugcucucuggugaaagugccgccaucuuuugaguguua

SEQ ID NO: 538 - RNA, 150 nt, Homo sapiens
uggccgaagaacaugggugagaaguccugggcugucucagagccuggagugggggggcaggaggggcucagggagaaagugcauacagccccuggcccucucugcccuuccguccccugcucuuggccaugcugugcccaguccaggcca

SEQ ID NO: 539 - RNA, 99 nt, Homo sapiens
uggcuaagguguuggcucgggcuccccacugcaguuacccuccccucggcguuacugagcacugggggcuuucgggcucugcgucugcacagauacuuc

SEQ ID NO: 540 - RNA, 81 nt, Homo sapiens
uggcugcacucaacauucauugcugucgguggguuugagucugaaucaacucacugaucaaugaaugcaaacugcggacca

SEQ ID NO: 541 - RNA, 106 nt, Homo sapiens
ugggccgggagguugaacauccugcauagugcugccaggaaaucccuauuucauauaagagggggcuggcugguugcauauguaggaugucccaucucccagccca

SEQ ID NO: 542 - RNA, 97 nt, Homo sapiens
ugguaaauugaugcacccagugggggagccaggaaguauugauguuucugccaguuuagcgucaacacuugcugguuuccucucuggagcaucgcca

SEQ ID NO: 543 - RNA, 82 nt, Homo sapiens
ugguaccugaaaagaaguugcccauguuauuuucgcuuuauaugugacgaaacaaacauggugcacuucuuuuucgguauca

SEQ ID NO: 544 - RNA, 82 nt, Homo sapiens
ugguaccugaagagagguuuucuggguuucuguuucuuuaaugaggacgaaacacaccugguuaaccucuuuuccaguauca

SEQ ID NO: 545 - RNA, 82 nt, Homo sapiens
ugguaccugaagagagguuuucuggguuucuguuucuuuauugaggacgaaacacaccugguuaaccucuuuuccaguauca

SEQ ID NO: 546 - RNA, 82 nt, Homo sapiens
ugguacuugaagagaaguuguucgugguggauucgcuuuacuuaugacgaaucauucacggacaacacuuuuuucaguacca

SEQ ID NO: 547 - RNA, 81 nt, Homo sapiens
ugguacuuggagagaggcuggccgugaugaauucgauucaucaaagcgagucauacacggcucuccucucuuuuaguguca

SEQ ID NO: 548 - RNA, 87 nt, Homo sapiens
uggugugguggggcagcugguguugugaaucaggccguugccaaucagagaacggcuacuucacaacaccagggccacaccacacua

SEQ ID NO: 549 - RNA, 83 nt, Homo sapiens
ugguguuaucaaguguaacagcaacuccauguggacuguguaccaauuuccaguggagaugcuguuacuuuugaugguuacca

SEQ ID NO: 550 - RNA, 100 nt, Homo sapiens
uguucagaaagucuguuguuguaaacauccccgacuggaagcuguaagacacagcuaagcuuucagucagauguuugcugcuaccggcuauucacagaca

SEQ ID NO: 551 - RNA, 97 nt, Homo sapiens
uguugcgcuccucucagucccugagacccuaacuugugauguuuaccguuuaaauccacggguuaggcucuugggagcugcgagucgugcuuuugca

SEQ ID NO: 552 - RNA, 65 nt, Homo sapiens
uuaguugguggagugauuugucugguuaauuccaauaaccaacgagacucuggcaugcuaacuag

SEQ ID NO: 553 - RNA, 105 nt, Homo sapiens
uuauuuuaccuucuuguauaagcacugugcuaaaauugcagacacuaggaccaugucuugguuuuugcaauaaugcuagcagaguacacacaagaagaaaaguaa

SEQ ID NO: 554 - RNA, 87 nt, Homo sapiens
uucacucuagugcuuuauggcuuuuuauuccuaugugauaguaauaaagucucauguagggauggaagccaugaaauacauugugaa

SEQ ID NO: 555 - RNA, 86 nt, Homo sapiens
uucucucugcuuucagcuucuuuacaguguugccuuguggcauggaguucaagcagcauuguacagggcuaucaaagcacagagag

SEQ ID NO: 556 - RNA, 99 nt, Homo sapiens
uucugugaccccuuagaggaugacugauuucuuuugguguucagagucaauauaauuuucuagcaccaucugaaaucgguuauaaugauuggggaagag

SEQ ID NO: 557 - RNA, 84 nt, Homo sapiens
uugacuuagcuggguaguggggaacccuuccaugaggaguagaacacuccuuaugcaagauucccuucuaccuggcuggguugg

SEQ ID NO: 558 - RNA, 102 nt, Homo sapiens
uugcucuaucagagugagguaguagauuguauaguugugggguagugauuuuacccuguucaggagauaacuauacaaucuauugccuucccugaggaguag

SEQ ID NO: 559 - RNA, 109 nt, Homo sapiens
uugugacugcaugcucccagguugagguaguagguuguauaguuuagaauuacaucaagggagauaacuguacagccuccuagcuuuccuugggucuugcacuaaacaa

SEQ ID NO: 560 - RNA, 86 nt, Homo sapiens
uuugguauuuaaaagguggauauuccuucuauguuuauguuauuuaugguuaaacauagaggaaauuccacguuuucaguaucaaa

SEQ ID NO: 561 - RNA, 63 nt, Homo sapiens
uuuucgguuaucaugguaccgaugcuguauaucugaaagguacaguacugugauaacugaaga

(19) United States
(12) Patent Application Publication
(10) Pub. No.: US 2009/0186353 A1
(43) Pub. Date: Jul. 23, 2009

(54) CANCER-RELATED NUCLEIC ACIDS

(75) Inventors: Ranit Aharonov, Rehovot (IL); Nitzan Rosenfeld, Rehovot (IL); Hila Benjamin, Rehovot (IL)

Correspondence Address: FOLEY AND LARDNER LLP;SUITE 500, 3000 K STREET NW, WASHINGTON, DC, 20007, US

(73) Assignee: Rosetta Genomics Ltd.

(21) Appl. No.: 12/298,065

(22) PCT Filed: May 4, 2007
(86) PCT No.: PCT/IB07/03116

§ 371 Date: Nov. 14, 2008

Related U.S. Application Data
(63) Continuation-in-part of application No. 11/418,718, filed on May 4, 2006, now abandoned.
Application No. 11/418,718 is a continuation-in-part of application No. 11/418,870, filed on May 4, 2006.
Application No. 11/418,870 is a continuation-in-part of application No. 11/130,645, filed on May 16, 2005.
Application No. PCT/IB07/03116 is a continuation-in-part of application No. 11/418,875, filed on May 4, 2006.
Application No. 11/418,875 is a continuation-in-part of application No. 11/130,645, filed on May 16, 2005.
Application No. PCT/IB07/03116 is a continuation-in-part of application No. 11/429,720, filed on May 8, 2006.
Application No. 11/429,720 is a continuation of application No. 11/130,645, filed on May 16, 2005.
(60) Provisional application No. 60/665,094, filed on Mar. 25, 2005.
(60) Provisional application No. 60/522,457, filed on Oct. 4, 2004.
(60) Provisional application No. 60/665,094, filed on Mar. 25, 2005.
(60) Provisional application No. 60/665,094, filed on Mar. 25, 2005.
(60) Provisional application No. 60/522,457, filed on Oct. 4, 2004.
(60) Provisional application No. 60/825,247, filed on Sep. 11, 2006.

(51) Int. Cl. C12Q 1/68; C07H 21/02

(52) U.S. Cl. 435/6; 536/24.31

(57) ABSTRACT

Described herein are polynucleotides associated with specific types of cancers. The polynucleotides are miRNAs, miRNA precursors, and associated nucleic acids. Methods and compositions are described that can be used for diagnosis, prognosis, and treatment of various cancers.

### FIELD OF THE INVENTION

The invention relates in general to microRNA molecules associated with specific cancers, as well as various nucleic acid molecules relating thereto or derived therefrom.

### BACKGROUND OF THE INVENTION

In recent years, microarray analysis of gene expression patterns has provided a way to improve the diagnosis and risk stratification of many cancers. Extensive differences in gene expression patterns has identified molecularly distinct subtypes of cancer, that were previously considered homogeneous based on classical diagnostic methods. Such molecular subtypes are often associated with different clinical outcomes. Global gene expression pattern can also be examined for features that correlate with clinical behavior to create prognostic signatures.

The expression of many MicroRNAs are altered in numerous types of human cancer. mRNAs are short RNA oligonucleotides of approximately 22 nucleotides that are involved in gene regulation. mRNAs regulate gene expression by targeting mRNAs for cleavage or translational repression. Although miRNAs are present in a wide range of species including C. elegans, Drosophila and humans, they have only recently been identified. More importantly, the role of miRNAs in the development and progression of disease has only recently become appreciated. Deregulated miRNA expression is implicated in onset and progression of different diseases including, but not limited to embryonic malformations and cancers. Alterations in miRNA expression have been implicated in playing a causative role in tumor progression.

The search for biomarkers for the early detection and diagnosis of various cancers has met with little success. Much emphasis has been placed on the discovery and characterization of a unique tumor marker. However, in the majority of cancers no marker has been identified that has adequate sensitivity or specificity to be clinically useful, although a combination of multiple markers has been shown to increase diagnostic accuracy. There is an unmet need for specific and accurate markers associated with specific types of cancers.

### SUMMARY OF THE INVENTION

The present invention provides specific nucleic acid sequences that may be used for the identification and diagnosis of various cancers including but not limited to small intestine cancer, bladder cancer, prostate cancer, lung cancer, thyroid cancer, uterus cancer, liver cancer, kidney cancer, breast cancer, stomach cancer, testicular cancer, cervical cancer, esophageal cancer, gallbladder cancer, ovarian cancer, melanoma and colon cancer. The nucleic acid sequences can also be used for prognosis evaluation of a subject based on the expression pattern of a biological sample.

An isolated nucleic acid is also provided. The nucleic acid may comprise a sequence of any of SEQ ID NOS: 1-561, the complement thereof, or a sequence at least 81% identical to 21 contiguous nucleotides thereof. The nucleic acid may be from about 51 to about 250 nucleotides in length. The nucleic acid may comprise a modified base.

A probe comprising the nucleic acid is also provided. A composition comprising the probe is also provided. A biochip comprising the probe is also provided.

A method for detecting a cancer-associated nucleic acid is also provided. A biological sample may be provided from which the level of a nucleic acid may be measured. The nucleic acid may comprise a sequence of any of SEQ ID NOS: 1-561. The nucleic acid may also comprise a sequence at least about 81% identical to about 21 contiguous nucleotides of any of SEQ ID NOS: 1-561. A level of the nucleic acid higher or lower than that of a control may be indicative of a specific cancer.

A method of diagnosing a subject with a specific cancer is also provided. The method comprising: providing a biological sample from the subject; and measuring the level of a nucleic acid sequence selected from the group consisting of SEQ ID NOS: 1-561, wherein a level of the nucleic acid different from a control is indicative of said cancer.

The invention further provides a method to distinguish between small intestine carcinoid tumor and small intestine stromal tumor, the method comprising determining a level of a nucleic acid sequence as set forth in Table 3 in a small intestine tumor sample obtained from a subject.

The invention further provides a method to distinguish between lung adenocarcinoma and squamous cell carcinoma, the method comprising determining a level of a nucleic acid sequence as set forth in Table 3 in a lung sample.

The invention further provides a method to distinguish between lung undifferentiated large cell carcinoma and squamous cell carcinoma, the method comprising determining a level of a nucleic acid sequence as set forth in Table 3 in a lung sample.

The invention further provides a method to distinguish between prostate adenocarcinoma and benign prostatic hyperplasia (BPH), the method comprising determining a level of a nucleic acid sequence as set forth in Table 3 in a prostate sample.

The invention further provides a method to distinguish between high grade carcinoma, medium grade carcinoma and low grade carcinoma, the method comprising determining a level of a nucleic acid sequence as set forth in Table 3 in a tumor sample obtained from a subject.

The invention further provides a method to distinguish between primary liver cancer and liver metastasis, the method comprising determining a level of a nucleic acid sequence as set forth in Table 3 in a liver sample.

A method for identifying a compound that modulates expression of a cancer-associated miRNA is also provided. A cell is provided that is capable of expressing a nucleic acid comprising a sequence of any of SEQ ID NOS: 1-561. A cell may also be provided that is capable of expressing a nucleic acid comprising a sequence at least about 81% identical to about 21 contiguous nucleotides of any of SEQ ID NOS: 1-561. The cell may be contacted with a candidate modulator. The level of expression of the nucleic acid may then be measured. A difference in the level of the nucleic acid compared to a control identifies the compound as a modulator of expression of the miRNA.

A method of treating or preventing cancer in a subject in need thereof is also provided. The subject may suffer from a specific cancer as set forth in Table 1. The subject may be administered an effective amount of a composition comprising a nucleic acid. The nucleic acid may comprise a sequence of any of SEQ ID NOS: 1-561, the complement thereof, or a sequence at least 81% identical to 21 contiguous nucleotides thereof. The nucleic acid may also comprise the sequence of any of SEQ ID NOS: 1-561, the complement thereof, or a sequence at least 63% identical to 81 contiguous nucleotides thereof. The nucleic acid may be from about 51 to about 250 nucleotides in length. The nucleic acid may comprise a modified base.

### BRIEF DESCRIPTION OF THE TABLES

miR name: is the miRBase registry name (release 9.1). Names which are not presented in the miRBase registry were cloned in Rosetta Genomics.

HIDs—Hairpins ID are the SEQ ID NOS of the microRNA hairpin sequences. Some microRNA can be derived from more than one hairpin, positioned at different genomic locations; in those cases the SEQ ID NOS of all related hairpins are presented.

MID—Mir ID, the SEQ ID NO of the mature microRNA sequence.

RID—Row ID. in decisions table, running number that is an identifier for further use as a reference.

TID—Tissue ID, the numeric code of the tissue tested, as described in Table 1.

Signal—the normalized mean signal read from the chip.

DID—Decision ID, describing the comparisons of one group of tissue(s) to another group of tissue(s) presented in Table 3.

p-value—2-taied, un-paired, t-test that compare the means if Group 1 and Group 2. Only p-values which are lower than 0.05 are presented, When “0” is presented, the p-value is lower than 1.0e-38.

n1—the number of samples tested in Group 1.

n2—the number of samples tested in Group 2.

Mean1—the mean microRNA score of all the samples in Group 1.

Mean2—the mean microRNA score of all the samples in Group 2.

### BRIEF DESCRIPTION OF THE DRAWINGS

FIG. 1 shows the average normalized signal and standard deviation (STD) divided by the square root of the number of samples (n) of hsa-miR-126 (SEQ ID NO:204) in two lung sample sets: lung tumors and normal lungs. T-test p-value: 1.73e-008.

FIG. 2 shows the average normalized signal and standard deviation (STD) divided by the square root of the number of samples (n) of hsa-miR-200c (SEQ ID NO: 156) in two liver sample sets: primary tumors and metastasis. T-test p-value: 1.48E-09.

FIG. 3 shows the average normalized signal and standard deviation (STD) divided by the square root of the number of samples (n) of hsa-miR-375 (SEQ ID NO:273) in two small intestine sample sets: stromal tumors and carcinoid tumors. T-test p-value: 6.68E-08.

FIG. 4 shows the average normalized signal and standard deviation (STD) divided by the square root of the number of samples (n) of 43_{—}5 (SEQ ID NO:215) in two bladder cancer sample sets: high grade tumors and low grade tumors. T-test p-value: 2.49E-08.

### DETAILED DESCRIPTION

Nucleic acids are provided related to miRNAs and precursors thereto. Such nucleic acids may be useful for diagnostic and prognostic purposes. Also provided are methods and compositions that may be useful, among other things, for diagnostic and prognostic purposes.

Other aspects of the invention will become apparent to the skilled artisan by the following description of the invention.

### 1. DEFINITIONS

Before the present compounds, products and compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

“About” as used herein may mean +/−10%.

“Aberrant proliferation” as used herein may mean cell proliferation that deviates from the normal, proper, or expected course. For example, aberrant cell proliferation may include inappropriate proliferation of cells whose DNA or other cellular components have become damaged or defective. Aberrant cell proliferation may include cell proliferation whose characteristics are associated with an indication caused by, mediated by, or resulting in inappropriately high levels of cell division, inappropriately low levels of apoptosis, or both. Such indications may be characterized, for example, by single or multiple local abnormal proliferations of cells, groups of cells, or tissue(s), whether cancerous or non-cancerous, benign or malignant.

“Antisense” as used herein may mean nucleotide sequences that are complementary to a specific sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand may combine with natural sequences produced by the cell to form duplexes. These duplexes then block either the further transcription or translation. In this manner, mutant phenotypes may be generated.

“Animal” as used herein may mean fish, amphibians, reptiles, birds, and mammals, such as mice, rats, rabbits, goats, cats, dogs, cows, apes and humans.

“Attached” or “immobilized” as used herein to refer to a probe and a solid support may mean that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

“Biological sample” as used herein may mean a sample of biological tissue or fluid that comprises nucleic acids. Such samples include, but are not limited to, tissue or fluid isolated from animals. Biological samples may also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from animal or patient tissues. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

“Cancer” as used herein may mean all types of cancerous growths or oncogenic processes, metastatic tissues or malignantly transformed cells, tissues, or organs, irrespective of histopathologic type or stage of invasiveness. Examples of cancers include but are nor limited to solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumor, non-small cell lung, oat cell, papillary, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adeno-carcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neurofibromatosis, and cervical dysplasia, and other conditions in which cells have become immortalized or transformed.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Diagnostic” as used herein may mean classifying a pathology or a symptom, determining a severity of the pathology (grade or stage), monitoring pathology progression, forecasting an outcome of a pathology and/or prospects of recovery.

“Differential expression” as used herein may mean qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

“Gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

“Host cell” as used herein may be a naturally occurring cell or a transformed cell that may contain a vector and may support replication of the vector. Host cells may be cultured cells, explants, cells in vivo, and the like. Host cells may be prokaryotic cells such as E. coli, or eukaryotic cells such as yeast, insect, amphibian, or mammalian cells, such as CHO and HeLa.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Label” as used herein may mean a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Predisposition” as used herein may mean an increased likelihood that an individual will have a disorder. Although a subject with a predisposition does not yet have the disorder, there exists an increased propensity to the disease.

“Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Selectable marker” as used herein may mean any gene which confers a phenotype on a host cell in which it is expressed to facilitate the identification and/or selection of cells which are transfected or transformed with a genetic construct. Representative examples of selectable markers include the ampicillin-resistance gene (Amp^{r}), tetracycline-resistance gene (Tc^{r}), bacterial kanamycin-resistance gene (Kan^{r}), zeocin resistance gene, the AURI-C gene which confers resistance to the antibiotic aureobasidin A, phosphinothricin-resistance gene, neomycin phosphotransferase gene (nptII), hygromycin-resistance gene, beta-glucuronidase (GUS) gene, chloramphenicol acetyltransferase (CAT) gene, green fluorescent protein (GFP)-encoding gene and luciferase gene.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” as used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

“Subject” as used herein may mean a mammal, including both human and other mammals.

“Target” as used herein may mean a polynucleotide that may be bound by one or more probes under stringent hybridization conditions.

“Terminator” as used herein may mean a sequence at the end of a transcriptional unit which signals termination of transcription. A terminator may be a 3′-non-translated DNA sequence containing a polyadenylation signal, which may facilitate the addition of polyadenylate sequences to the 3′-end of a primary transcript. A terminator may be derived from sources including viral, bacterial, fungal, plants, insects, and animals. Representative examples of terminators include the SV40 polyadenylation signal, HSV TK polyadenylation signal, CYC1 terminator, ADH terminator, SPA terminator, nopaline synthase (NOS) gene terminator of Agrobacterium tumefaciens, the terminator of the Cauliflower mosaic virus (CaMV) 35S gene, the zein gene terminator from Zea mays, the Rubisco small subunit gene (SSU) gene terminator sequences, subclover stunt virus (SCSV) gene sequence terminators, rho-independent E. coli terminators, and the lacZ alpha terminator.

“Therapeutically effective amount” or “therapeutically efficient” as used herein in respect to a drug dosage, may refer to dosage that provides the specific pharmacological response for which the drug is administered in a significant number of subjects in need of such treatment.

The “therapeutically effective amount” may vary according, for example, the physical condition of the patient, the age of the patient and the severity of the disease.

“Treatment regimen” as used herein may mean a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relieve symptoms of the pathology yet results in incomplete cure of the pathology. It will be appreciated that in certain cases the treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

“Variant” used herein to refer to a nucleic acid may mean (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

“Wild type” as used herein the context of a sequence may refer to a coding, non-coding or interface sequence is an allelic form of sequence that performs the natural or normal function for that sequence. Wild type sequences include multiple allelic forms of a cognate sequence, for example, multiple alleles of a wild type sequence may encode silent or conservative changes to the protein sequence that a coding sequence encodes.

### 2. MicroRNA

A gene coding for a miRNA may be transcribed leading to production of a miRNA primary transcript known as the pri-miRNA. The pri-miRNA may form a hairpin with a stem and loop. The stem of the hairpin may comprise mismatched bases. The pri-miRNA may comprise several pri-miRNAs in a polycistronic structure.

The hairpin structure of the pri-miRNA may be recognized by Drosha, which is an RNase III endonuclease. Drosha may recognize terminal loops in the pri-miRNA and cleave approximately two helical turns into the stem to produce a 60-70 nt precursor known as the pre-miRNA. Drosha may cleave the pri-miRNA with a staggered cut typical of RNase III endonucleases yielding a pre-miRNA stem loop with a 5′ phosphate and ˜2 nucleotide 3′ overhang. Approximately one helical turn of the stem (˜10 nucleotides) extending beyond the Drosha cleavage site may be essential for efficient processing. The pre-miRNA may then be actively transported from the nucleus to the cytoplasm by Ran-GTP and the export receptor Ex-portion-5.

The pre-miRNA may be recognized by Dicer, which is also an RNase III endonuclease. Dicer may recognize the double-stranded stem of the pre-miRNA. Dicer may cleave off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. mRNA* sequences may be found in libraries of cloned miRNAs but typically at lower frequency than the miRNAs.

Although initially present as a double-stranded species with miRNA*, the miRNA may eventually become incorporated as a single-stranded RNA into a ribonucleoprotein complex known as the RNA-induced silencing complex (RISC). Various proteins can form the RISC, which can lead to variability in specifity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target nucleic acids based on high levels of complementarity between the miRNA and the mRNA, especially by nucleotides 2-7 of the miRNA. Only one case has been reported in animals where the interaction between the miRNA and its target was along the entire length of the miRNA. This was shown for mir-196 and Hox B8 and it was further shown that mir-196 mediates the cleavage of the Hox B8 mRNA (Yekta et al 2004, Science 304-594). Otherwise, such interactions are known only in plants (Bartel & Bartel 2003, Plant Physiol 132-709).

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et al 2005 Cell 120-15). Similarly, nucleotides 1-7 or 2-8 were used to identify and validate targets by Krek et al (2005, Nat Genet. 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut may be between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

### 3. NUCLEIC ACID

Nucleic acids are provided herein. The nucleic acid may comprise the sequence of SEQ ID NOS: 1-561 or variants thereof. The variant may be a complement of the referenced nucleotide sequence. The variant may also be a nucleotide sequence that is substantially identical to the referenced nucleotide sequence or the complement thereof. The variant may also be a nucleotide sequence that hybridizes under stringent conditions to the referenced nucleotide sequence, complements thereof, or nucleotide sequences substantially identical thereto.

The nucleic acid may have a length of from 10 to 250 nucleotides. The nucleic acid may have a length of at least 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 175, 200 or 250 nucleotides. The nucleic acid may be synthesized or expressed in a cell (in vitro or in vivo) using a synthetic gene described herein. The nucleic acid may be synthesized as a single strand molecule and hybridized to a substantially complementary nucleic acid to form a duplex. The nucleic acid may be introduced to a cell, tissue or organ in a single- or double-stranded form or capable of being expressed by a synthetic gene using methods well known to those skilled in the art, including as described in U.S. Pat. No. 6,506,559 which is incorporated by reference.

a. Pri-miRNA

The nucleic acid may comprise a sequence of a pri-miRNA or a variant thereof. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof. The sequence of the pri-miRNA may comprise the sequence of SEQ ID NOS: 1-561, the complement of a target gene binding site, or variants thereof.

The pri-miRNA may comprise a hairpin structure. The hairpin may comprise a first and second nucleic acid sequence that are substantially complementary. The first and second nucleic acid sequence may be from 37-50 nucleotides. The first and second nucleic acid sequence may be separated by a third sequence of from 8-12 nucleotides. The hairpin structure may have a free energy less than −25 Kcal/mole as calculated by the Vienna algorithm with default parameters, as described in Hofacker et al., Monatshefte f. Chemie 125: 167-188 (1994), the contents of which are incorporated herein. The hairpin may comprise a terminal loop of 4-20, 8-12 or 10 nucleotides. The pri-miRNA may comprise at least 19% adenosine nucleotides, at least 16% cytosine nucleotides, at least 23% thymine nucleotides and at least 19% guanine nucleotides.

b. Pre-miRNA

The nucleic acid may also comprise a sequence of a pre-miRNA or a variant thereof. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 1-561, the complement of a target gene binding site, or variants thereof.

c. MiRNA

The nucleic acid may also comprise a sequence of a miRNA (including miRNA*) or a variant thereof. The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be from the first 13-33 nucleotides of the pre-miRNA. The sequence of the miRNA may also be from the last 13-33 nucleotides of the pre-miRNA. The sequence of the miRNA may comprise the sequence of SEQ ID NOS: 1-274, the complement of a target gene binding site, or variants thereof.

d. Anti-miRNA

The nucleic acid may also comprise a sequence of an anti-miRNA that is capable of blocking the activity of a miRNA or miRNA*, such as by binding to the pri-miRNA, pre-miRNA, miRNA or miRNA* (e.g. antisense or RNA silencing), or by binding to the target binding site. The anti-miRNA may comprise a total of 5-100 or 10-60 nucleotides. The anti-miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the anti-miRNA may comprise (a) at least 5 nucleotides that are substantially complementary to the 5′ of a miRNA and at least 5-12 nucleotides that are substantially identical to the flanking regions of the target site from the 5′ end of the miRNA, for the purposes of binding to a miRNA and repressing its activity; or (b) at least 5-12 nucleotides that are substantially identical to the 3′ of a miRNA and at least 5 nucleotide that are substantially complementary to the flanking region of the target site from the 3′ end of the miRNA, for the purposes of inhibiting the ability of a miRNA to bind to its target. The sequence of the anti-miRNA may comprise the complement of SEQ ID NOS: 1-561, the complement of a target gene binding site, or variants thereof.

e. Binding Site of Target

The nucleic acid may also comprise a sequence of a target miRNA binding site, or a variant thereof. The target site sequence may comprise a total of 5-100 or 10-60 nucleotides. The target site sequence may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 nucleotides. The target site sequence may comprise at least 5 nucleotides of the sequence of SEQ ID NOS: 1-274.

### 4. SYNTHETIC GENE

A synthetic gene is also provided comprising a nucleic acid described herein operably linked to a transcriptional and/or translational regulatory sequence. The synthetic gene may be capable of modifying the expression of a target gene with a binding site for a nucleic acid described herein. Expression of the target gene may be modified in a cell, tissue or organ. The synthetic gene may be synthesized or derived from naturally-occurring genes by standard recombinant techniques. The synthetic gene may also comprise terminators at the 3′-end of the transcriptional unit of the synthetic gene sequence. The synthetic gene may also comprise a selectable marker.

### 5. VECTOR

A vector is also provided comprising a nucleic acid described herein, such as a pri-miRNA, pre-miRNA, miRNA, anti-miRNA, target gene binding site, or synthetic gene. The vector may be an expression vector. An expression vector may comprise additional elements. For example, the expression vector may have two replication systems allowing it to be maintained in two organisms, e.g., in one host cell for expression and in a second host cell (e.g., bacteria) for cloning and amplification. For integrating expression vectors, the expression vector may contain at least one sequence homologous to the host cell genome, and preferably two homologous sequences that flank the expression construct. The integrating vector may be directed to a specific locus in the host cell by selecting the appropriate homologous sequence for inclusion in the vector. The vector may also comprise a selectable marker gene to allow the selection of transformed host cells.

### 6. HOST CELL

A host cell is also provided comprising a vector, synthetic gene or nucleic acid described herein. The cell may be a bacterial, fungal, plant, insect or animal cell.

### 7. PROBES

A probe is also provided comprising a nucleic acid described herein. Probes may be used for screening and diagnostic methods, as outlined below. The probe may be attached or immobilized to a solid substrate, such as a biochip.

The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may further comprise a linker sequence of from 10-60 nucleotides.

### 8. BIOCHIP

A biochip is also provided. The biochip may comprise a solid substrate comprising an attached probe or plurality of probes described herein. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder.

The probes may be attached to the biochip in a wide variety of ways, as will be appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linkers. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

### 9. EXPRESSION ANALYSIS

A method of identifying a nucleic acid associated with a specific cancer or a related pathological condition is also provided. The method comprises measuring a level of the nucleic acid in a sample that is different than the level of a control. Detection may be performed by contacting the sample with a probe or biochip described herein and detecting the amount of hybridization. PCR may be used to amplify nucleic acids in the sample, which may provide higher sensitivity.

The level of the nucleic acid in the sample may also be compared to a control cell (e.g., a normal cell) to determine whether the nucleic acid is differentially expressed (e.g., overexpressed or underexpressed). The ability to identify miRNAs that are differentially expressed in pathological cells compared to a control can provide high-resolution, high-sensitivity datasets which may be used in the areas of diagnostics, prognostics, therapeutics, drug development, pharmacogenetics, biosensor development, and other related areas. An expression profile generated by the current methods may be a “fingerprint” of the state of the sample with respect to a number of miRNAs. While two states may have any particular miRNA similarly expressed, the evaluation of a number of miRNAs simultaneously allows the generation of a gene expression profile that is characteristic of the state of the cell. That is, normal tissue may be distinguished from cancerous tissue. By comparing expression profiles of tissue in known different disease states, information regarding which miRNAs are associated in each of these states may be obtained. Then, diagnosis may be performed or confirmed to determine whether a tissue sample has the expression profile of normal or disease tissue. This may provide for molecular diagnosis of related conditions.

The expression level of a cancer-associated nucleic acid is information in a number of ways. For example, a differential expression of a cancer-associated nucleic acid compared to a control may be used as a diagnostic that a patient suffers from the cancer. Expression levels of a cancer-associated nucleic acid may also be used to monitor the treatment and cancer state of a patient. Furthermore, expression levels of a cancer-associated miRNA may allow the screening of drug candidates for altering a particular expression profile or suppressing an expression profile associated with cancer.

A target nucleic acid may be detected and levels of the target nucleic acid measured by contacting a sample comprising the target nucleic acid with a biochip comprising an attached probe sufficiently complementary to the target nucleic acid and detecting hybridization to the probe above control levels.

The target nucleic acid may also be detected by immobilizing the nucleic acid to be examined on a solid support such as nylon membranes and hybridizing a labeled probe with the sample. Similarly, the target nucleic may also be detected by immobilizing the labeled probe to a solid support and hybridizing a sample comprising a labeled target nucleic acid. Following washing to remove the non-specific hybridization, the label may be detected.

The target nucleic acid may also be detected in situ by contacting permeabilized cells or tissue samples with a labeled probe to allow hybridization with the target nucleic acid. Following washing to remove the non-specifically bound probe, the label may be detected.

These assays can be direct hybridization assays or can comprise sandwich assays, which include the use of multiple probes, as is generally outlined in U.S. Pat. Nos. 5,681,702; 5,597,909; 5,545,730; 5,594,117; 5,591,584; 5,571,670; 5,580,731; 5,571,670; 5,591,584; 5,624,802; 5,635,352; 5,594,118; 5,359,100; 5,124,246; and 5,681,697, each of which is hereby incorporated by reference.

A variety of hybridization conditions may be used, including high, moderate and low stringency conditions as outlined above. The assays may be performed under stringency conditions which allow hybridization of the probe only to the target. Stringency can be controlled by altering a step parameter that is a thermodynamic variable, including, but not limited to, temperature, formamide concentration, salt concentration, chaotropic salt concentration pH, or organic solvent concentration.

Hybridization reactions may be accomplished in a variety of ways. Components of the reaction may be added simultaneously, or sequentially, in different orders. In addition, the reaction may include a variety of other reagents. These include salts, buffers, neutral proteins, e.g., albumin, detergents, etc. which may be used to facilitate optimal hybridization and detection, and/or reduce non-specific or background interactions. Reagents that otherwise improve the efficiency of the assay, such as protease inhibitors, nuclease inhibitors and anti-microbial agents may also be used as appropriate, depending on the sample preparation methods and purity of the target.

a. Diagnostic

A method of diagnosis is also provided. The method comprises detecting a differential expression level of a specific cancer-associated nucleic acid in a biological sample. The sample may be derived from a patient. Diagnosis of a cancer state in a patient may allow for prognosis and selection of therapeutic strategy. Further, the developmental stage of cells may be classified by determining temporarily expressed cancer-associated nucleic acids.

In situ hybridization of labeled probes to tissue arrays may be performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

b. Drug Screening

A method of screening therapeutics is also provided. The method comprises contacting a pathological cell capable of expressing a cancer related nucleic acid with a candidate therapeutic and evaluating the effect of a drug candidate on the expression profile of the cancer associated nucleic acid. Having identified the differentially expressed nucleic acid, a variety of assays may be executed. Test compounds may be screened for the ability to modulate gene expression of the cancer associated nucleic acid. Modulation includes both an increase and a decrease in gene expression.

The test compound or drug candidate may be any molecule, e.g., protein, oligopeptide, small organic molecule, polysaccharide, polynucleotide, etc., to be tested for the capacity to directly or indirectly alter the disease phenotype or the expression of the disease associated nucleic acid. Drug candidates encompass numerous chemical classes, such as small organic molecules having a molecular weight of more than 100 and less than about 500, 1,000, 1,500, 2,000 or 2,500 daltons. Candidate compounds may comprise functional groups necessary for structural interaction with proteins, particularly hydrogen bonding, and typically include at least an amine, carbonyl, hydroxyl or carboxyl group, preferably at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Combinatorial libraries of potential modulators may be screened for the ability to bind to the disease associated nucleic acid or to modulate the activity thereof. The combinatorial library may be a collection of diverse chemical compounds generated by either chemical synthesis or biological synthesis by combining a number of chemical building blocks such as reagents. Preparation and screening of combinatorial chemical libraries is well known to those of skill in the art. Such combinatorial chemical libraries include, but are not limited to, peptide libraries encoded peptides, benzodiazepines, diversomers such as hydantoins, benzodiazepines and dipeptide, vinylogous polypeptides, analogous organic syntheses of small compound libraries, oligocarbamates, and/or peptidyl phosphonates, nucleic acid libraries, peptide nucleic acid libraries, antibody libraries, carbohydrate libraries, and small organic molecule libraries.

### 10. GENE SILENCING

A method of reducing expression of a target gene in a cell, tissue or organ is also provided. Expression of the target gene may be reduced by expressing a nucleic acid described herein that comprises a sequence substantially complementary to one or more binding sites of the target mRNA. The nucleic acid may be a miRNA or a variant thereof. The nucleic acid may also be pri-miRNA, pre-miRNA, or a variant thereof, which may be processed to yield a miRNA. The expressed miRNA may hybridize to a substantially complementary binding site on the target mRNA, which may lead to activation of RISC-mediated gene silencing. An example for a study employing over-expression of miRNA is Yekta et al 2004, Science 304-594, which is incorporated herein by reference. One of ordinary skill in the art will recognize that the nucleic acids described herein may also be used to inhibit expression of target genes or inhibit activity of miRNAs using antisense methods well known in the art, as well as RNAi methods described in U.S. Pat. Nos. 6,506,559 and 6,573,099, which are incorporated by reference.

The target of gene silencing may be a protein that causes the silencing of a second protein. By repressing expression of the target gene, expression of the second protein may be increased. Examples for efficient suppression of miRNA expression are the studies by Esau et al 2004 JBC 275-52361; and Cheng et al 2005 Nucleic Acids Res. 33-1290, which is incorporated herein by reference.

### 11. GENE ENHANCEMENT

A method of increasing expression of a target gene in a cell, tissue or organ is also provided. Expression of the target gene may be increased by expressing a nucleic acid described herein that comprises a sequence substantially complementary to a pri-miRNA, pre-miRNA, miRNA or a variant thereof. The nucleic acid may be an anti-miRNA. The anti-miRNA may hybridize with a pri-miRNA, pre-miRNA or miRNA, thereby reducing its gene repression activity. Expression of the target gene may also be increased by expressing a nucleic acid that is substantially complementary to a portion of the binding site in the target gene, such that binding of the nucleic acid to the binding site may prevent miRNA binding.

### 12. THERAPEUTIC

A method for treating cancer is also provided. The cancer may be treated in vivo or ex vivo. The compositions of the present invention may be combined with a chemotherapeutic agent, a combination of chemotherapeutic agents and/or radiotherapy.

In general, the nucleic acid molecules described herein may be used as a modulator of the expression of genes which are at least partially complementary to said nucleic acid. Further, miRNA molecules may act as target for therapeutic screening procedures, e.g. inhibition or activation of miRNA molecules might modulate a cellular differentiation process, e.g. proliferation or apoptosis.

Furthermore, existing miRNA molecules may be used as starting materials for the manufacture of sequence-modified miRNA molecules, in order to modify the target-specificity thereof, e.g. an oncogene, a multidrug-resistance gene or another therapeutic target gene. Further, miRNA molecules can be modified, in order that they are processed and then generated as double-stranded siRNAs which are again directed against therapeutically relevant targets. Furthermore, miRNA molecules may be used for tissue reprogramming procedures, e.g. a differentiated cell line might be transformed by expression of miRNA molecules into a different cell type or a stem cell.

### 13. COMPOSITIONS

A pharmaceutical composition is also provided. The composition may comprise a nucleic acid described herein and optionally a pharmaceutically acceptable carrier. The compositions may be used for diagnostic or therapeutic applications. The pharmaceutical composition may be administered by known methods, including wherein a nucleic acid is introduced into a desired target cell in vitro or in vivo.

The composition can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. The composition may also be locally delivered by direct injection or by use of an infusion pump. Other routes of delivery include, but are not limited to oral (tablet or pill form) and/or intrathecal delivery (Gold, 1997, Neuroscience, 76, 1153-1158). Other approaches for delivery include the use of various transport and carrier systems, for example, through the use of conjugates and biodegradable polymers. Other descriptions of nucleic acid delivery and administration are provided for example in WO93/23569, WO99/05094, WO99/04819, WO94/02595 and Akhtar et al., (Trends Cell Bio. 2,139,1992).

The composition can be introduced into tissues or host cells by any number of routes, including viral infection, microinjection, or fusion of vesicles. Jet injection may also be used for intramuscular administration, as described by Furth et al. (Anal Biochem 115 205:365-368, 1992). The composition can be coated onto gold microparticles, and delivered intradermally by a particle bombardment device, or “gene gun” as described in the literature (see, for example, Tang et al. Nature 356:152-154, 1992), where gold microprojectiles are coated with the DNA, then bombarded into skin cells.

The compositions of the present invention can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants and aerosols. As such, administration of the agents can be achieved in various ways, including oral, buccal, rectal, parenteral, intraperitoneal, intradermal, transdermal, intracheal, etc.

### 14. KITS

A kit is also provided comprising a nucleic acid described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein. The kit may further comprise a software package for data analysis of expression profiles.

For example, the kit may be a kit for the amplification, detection, identification or quantification of a target nucleic acid sequence. The kit may comprise a poly(T) primer, a forward primer, a reverse primer, and a probe.

### 15. METHOD OF SYNTHESIS

A method of synthesizing the reverse-complement of a target nucleic acid is also provided. The reverse complement may be synthesized according to methods outlined in U.S. patent Ser. No. 11/384,049, the contents of which are incorporated herein by reference.

### 16. METHOD OF DETECTION

A method of detecting a target nucleic acid in a biological sample is also provided. The target nucleic acid may be detected according to methods outlined in U.S. patent Ser. No. 11/384,049, the contents of which are incorporated herein by reference.

a. Detectable Malignancies

Liver cancer: Primary liver cancer is the fifth most common cancer worldwide. Hepatocellular carcinoma (HCC) accounts for 80% of all liver cancer and the rates of HCC have increased by over 70% in the last two decades in the U.S. The fatality ratio (mortality/incidence) of liver cancer is approximately 1, indicating that the majority of patients live less than a year. Late diagnosis due to lack of clinical symptoms is one of the main reasons for the high fatality ratio. Liver cancer can result from both viral infection and chemical exposure. Known risk factors include hepatitis B and C virus infection. It is not known whether distinct routes to liver cancer affect the same or different cellular pathways. No mutational model has yet been developed for liver cancer as it has been for other cancers. The molecular events that precede neoplastic transformation of the liver are not well understood. With no clearly identified cause, successful treatment options are lacking.

Nearly any primary tumor site can deposit metastases in the liver, since the liver filters blood from throughout the body. Most discussions related to the treatment of metastatic tumors in the liver focus on those originating from the colon. In fact, the most common cause of death from colorectal cancer is liver metastasis.

Up to 50% of liver metastases are of colorectal cancer origin, while the remainder metastasizes from a wide variety of primary cancer sites including sarcomas, breast and kidney, as well as neuroendocrine tumors.

HCC may be solitary or multicentric, and it may mimic liver metastases. Furthermore hemangiomas and liver metastases are often confused in imaging methods. In general, the imaging appearances of liver metastases are nonspecific, and biopsy specimens are required for histological diagnosis. Various biochemical markers have been proposed to indicate liver metastases. However, the diagnostic accuracy of tumor markers has not yet been defined.

Lung cancer: Lung cancer is the primary cause of cancer death among both men and women in the U.S., with an estimated 172,000 new cases being reported in 1994. The five-year survival rate among all lung cancer patients, regardless of the stage of disease at diagnosis, is only 13%. This contrasts with a five-year survival rate of 46% among cases detected while the disease is still localized. However, only 16% of lung cancers are discovered before the disease has spread. Lung cancers are broadly classified into small cell or non-small cell lung cancers. Non-small cell lung cancers are further divided into adenocarcinomas, bronchoalveolar-alveolar, squamous cell and large cell carcinomas. Approximately, 75-85 percents of lung cancers are non-small cell cancers and 15-25 percents are small cell cancers of the lung.

Lung large cell carcinoma often is discovered on a chest X-ray, computed tomography (CT) or magnetic resonance imaging (MRI) scans, This helps to find the best place to obtain a sample of the tumor to differentiate large cell carcinoma from other types of lung cancer such as small cell carcinoma or adenocarcinoma of the lung. The type of cancer affects the type of treatment the patient should get. Circulating tumor markers could impact non-small cell lung cancer (NSCLC) patient outcomes through improved screening diagnosis, staging and management. The search for biomarkers for the early detection and diagnosis of NSCLC has met with little success. Much emphasis has been placed on the discovery and characterization of a unique tumor marker. However, no marker has been identified that has adequate sensitivity or specificity to be clinically useful, although a combination of multiple markers has been shown to increase diagnostic accuracy.

Prostate cancer: Prostate cancer is a common disease in men above 50 years. Prostate cancer is the most frequent type of cancer in males, accounting for 25% of all cancers in the male population. Unfortunately, prostate carcinomas that are progressive in nature frequently have already metastasized by the time of clinical detection with available methods. Survival rates for individuals with metastatic prostate cancer are quite low. Between these two extremes are patients with prostate tumors that will metastasize during their lifetimes, but have not yet done so. For these patients, surgical removal of the prostate is curative and extends life expectancy. Therefore, accurate determination of which group a newly diagnosed patient falls into is critical in determining optimal treatment and patient survival.

Although clinical and pathologic stage and histological grading systems (e.g., Gleason's) have been used to indicate prognosis for groups of patients based on the degree of tumor differentiation or the type of glandular pattern (Diamond et al., J. Urol., 128: 729-734, 1982), these systems do not adequately predict the progression rate of the cancer. While the use of computer-system image analysis of histologic sections of primary lesions for “nuclear roundness” has been suggested as an aide in the management of individual patients (Diamond et al., 1982, J. Urol., 128:729-734), this method is of limited use in studying the progression of the disease.

Thyroid cancer: It is estimated that between 4-7% of the population harbors thyroid nodules. Literature reports that 5-30% of these nodules are malignant. The main diagnostic consideration in these cases is the exclusion of malignancy. Currently, the method offering the best preoperative prediction of the nature of these nodules is the fine needle aspiration biopsy of the lesion (FNA). Use of FNA resulted in decrease in number of thyroidectomies performed and increase in the yield of malignancy in resected lesions. However, instances of inadequate sampling of the lesion and overlapping cytological features of benign and malignant thyroid neoplasms are inherent limitations of this technique. The major FNA limitation is its inability to distinguish between well-differentiated follicular carcinomas and benign follicular adenomas. Patients with follicular thyroid neoplasm usually undergo thyroidectomy and about 15% have malignant lesion. Reliable preoperative diagnosis of benign lesion would greatly reduce number of unnecessary surgeries.

Bladder cancer: Bladder cancer is the fourth most prevalent human malignancy, with about 49,000 new cases and 9,700 deaths reported annually [Silverman, D. T. et al., Epidemiology of Bladder Cancer. In: “Hematology/Oncology Clinics of North America”. P. W. Kantoff et al. eds., W.B. Saunders Co., Philadelphia, p. 1 (1992)]. Ninety percent of bladder cancers are transitional cell carcinomas which are typically superficial at early stages but often become invasive at later stages, 5% of bladder cancers are squamous cell carcinomas (SCC), which are more prevalent in cases of chronic bladder irritation, and the remainders are rare tumors such as adenocarcinoma, carcinosarcoma. Bladder cancer is usually diagnosed via cystoscopy, an invasive procedure, wherein a fiber optic device is inserted into the bladder and lesions are detected visually by a urologist. Cystoscopy is performed on patients expressing the symptom complex characteristic of bladder cancer, i.e., hematuria, pain, or urinary obstruction. However, when symptoms appear, the tumor is usually progressed to a dangerous grade or stage. In addition, this type of macroscopic diagnostics fails to detect microscopic disease such as carcinoma in situ [Halachmi et al., (2001), Bladder cancer: genetic overview. Med. Sci. Monit. 7: 164-168].

Breast cancer: Breast cancer is by far the most common cancer in women worldwide. Current global incidence is in excess of 1,151,000 new cases diagnosed each year. Breast cancer incidence is highest in developed countries, particularly amongst populations of Northern European ethnic origin, and is increasing. In the United States the annual age-standardized incidence rate is approximately 131 cases per 100,000 population, more than three times the world average. Rates in Northern European countries are similarly high. In the year 2006 it is estimated that 214,640 new cases of invasive breast cancer will be diagnosed in the U.S.A. and 41,430 people will die from the disease. To this figure must be added a further 59,000 ductal and lobular carcinoma in situ diagnoses. From an individual perspective, the lifetime probability of developing breast cancer is 13.1% in U.S. women (i.e., 1 in 8 women will develop breast cancer during their lives). As with most cancers, early detection and appropriate treatment are important factors. Overall, the 5-year survival rate for breast cancer is 88%. However, in individuals presenting with regionally invasive or metastatic disease, the rate declines to 80% and 26%, respectively.

No universally successful method for the treatment or prevention of breast cancer is currently available. Management of breast cancer currently relies on a combination of early diagnosis (e.g., through breast screening procedures, e.g., mammography) and treatments using surgery, chemotherapy, radiotherapy and hormonal therapies. Increasingly, the focus is falling on the identification individuals who are at high risk for primary or recurrent breast cancer. Such individuals can be managed by more intensive screening, preventative chemotherapies or hormonal therapies and, in cases of individuals at extremely high risk, prophylactic surgery. There is a significant need, therefore, for improved diagnostic methods and identification of risk for breast cancer.

Colon cancer: The deaths by colon cancer are increasing. The number of deaths by colon cancer is the fourth large among male, and the second large among female deaths in all cancer deaths (Statistics of Japanese cancer deaths in 1999). According to an estimation of cancer patients in 2015 in Japan, number of colon cancer patients is estimated to be the first in both male and female. Global measures to counter colon cancer including secondary prevention are thus required, and mass screening of cancer may be one of the most effective methods. For the mass screening of cancer, it is important that the detection method is easy and non-invasive. The only non-invasive method now available is the method to examine existence of occult blood in feces, that is, the fecal occult blood test, and is used extensively as a standard method of the mass screening of colon cancer. However, the fecal occult blood test has rather low sensitivity and specificity (the sensitivity: 30 to 90%, the specificity: 70 to 98%), because appearance of hemoglobin in feces is not specific to tumor. Therefore, there is a shortcoming that quite a few false negatives and false positives exist.

Kidney cancer: The American Cancer Society predicted that there would be about 31,200 new cases of kidney cancer in the year 2000 in the United States alone. About 11,900 people, adults and children, will die from this disease each year. The cure rate of advanced stage kidney cancer is only fair and has improved little in the last two decades. Kidney cancer remains difficult to diagnose and treat effectively.

Small intestine cancer: Symptoms of cancer of the small intestine typically include pain or cramps in the middle of the abdomen, weight loss without dieting, and a lump in the abdomen or blood in the stool. Procedures used for detecting, diagnosing, monitoring, staging, and prognosticating cancer of the small intestine are of critical importance to the outcome of the patient. Patients diagnosed with early stage cancer generally have a much greater survival rate as compared to the survival rate for patients diagnosed with distant metastasized cancers. New diagnostic methods which are more sensitive and specific for detecting cancer of the small intestine are clearly needed.

Testicular cancer: Male germ cell tumors have been histologically categorized into seminomas, which retain germ cell characteristics, and nonseminomas which can display characteristics of embryonal differentiation. Both seminomas and nonseminomas are thought to initiate from a preinvasive stage designated carcinoma in situ (CIS) (Murty, et al., 1998, Sem. Oncol. 25:133-144). Testicular tumors develop from Leydig cells with high frequency in transgenic mice expressing human papilloma virus 16 (HPV16) E6 and E7 oncogenes (Kondoh, et al., 1991, J. Virol. 65:3335-3339; Kondoh, et al., 1994, J. Urol. 152:2151-2154).

The following examples are presented in order to more fully illustrate some embodiments of the invention. They should, in no way be construed, however, as limiting the broad scope of the invention.

### EXAMPLES

### Experimental Procedures

1. miRdicator™ Array Platform

Custom microarrays were produced by printing DNA oligonucleotide probes representing 688 miRNAs (Sanger database, version 9 and additional Rosetta Genomics validated and predicted miRs). Each probe carried up to a 22-nt linker at the 3‘end of the miRNA’s complement sequence in addition to an amine group used to couple the probes to coated glass slides. 20 μM of each probe were dissolved in 2×SSC+0.0035% SDS and spotted in triplicate on Schott Nexterion® Slide E coated microarray slides using a Genomic Solutions® BioRobotics MicroGrid II according the MicroGrid manufacturer's directions. 64 negative control probes were designed using the sense sequences of different miRNAs. Two groups of positive control probes were designed to hybridize to miRdicator™ array (1) synthetic spikes small RNA were added to the RNA before labeling to verify the labeling efficiency and (2) probes for abundant small RNA (e.g. small nuclear RNAs (U43, U49, U24, Z30, U6, U48, U44), 5.8s and 5s ribosomal RNA) were spotted on the array to verify RNA quality. The slides were blocked in a solution containing 50 mM ethanolamine, 1M Tris (pH 9.0) and 0.1% SDS for 20 min at 500 C., then thoroughly rinsed with water and spun dry.

2. Cy-Dye Labeling of miRNA for miRdicator™ Array

15 μg of total RNA was labeled by ligation of a RNA-linker p-rCrU-Cy-dye (Thomson et al., 2004, Nat Methods 1, 47-53) (Dharmacon) to the 3′-end with Cy3 or Cy5. The labeling reaction contained total RNA, spikes (20-0.1 fmoles), 500 ng RNA-linker-dye, 15% DMSO, 1× ligase buffer and 20 units of T4 RNA ligase (NEB) and proceeded at 40 C for 1 hr followed by 1 hr at 37° C. The labeled RNA was mixed with 3× hybridization buffer (Ambion), heated to 95° C. for 3 min and than added on top of the miRdicator™ array. Slides were hybridize 12-16 hr, followed by two washes with 1×SSC and 0.2% SDS and a final wash with 0.1×SSC.

The array was scanned using an Agilent Microarray Scanner Bundle G2565BA (resolution of 10 μm at 100% power). The data was analyzed using SpotReader software.

### 3. RNA Extraction

RNA was extracted from frozen or paraffin-embedded (FFPE) tissues originated from different tumor or normal tissues as indicated in Table 1, where TID—Tissue ID is the numeric code of each tissue or sub-type tissue.

Total RNA from frozen tissues was extracted with the miRvana miRNA isolation kit (Ambion) according to the manufacturer's instructions.

Total RNA from formalin fixed, paraffin-embedded (FFPE) tissues was extracted according to the following protocol:

1 ml Xylene (Biolab) was added to 1-2 mg tissue, incubated at 57° C. for 5 min and centrifuged for 2 min at 10,000 g. The supernatant was removed and 1 ml Ethanol (100%) (Biolab) was added. Following centrifugation for 10 min at 10,000 g, the supernatant was discarded and the washing procedure was repeated. Following air drying for 10-15 min, 500 μl Buffer B (NaCl 10 mM, Tris pH 7.6, 500 mM, EDTA 20 mM, SDS 1%) and 5 ul proteinase K (50 mg/ml) (Sigma) were added. Following incubation at 45° C. for 16 h, inactivation of the proteinase K at 100° C. for 7 min was preformed. Following extraction with acid phenol chloroform (1:1) (Sigma) and centrifugation for 10 min at maximum speed at 4° C., the upper phase was transferred to a new tube with the addition of 3 volumes of 100% Ethanol, 0.1 volume of NaOAc (BioLab) and 8 μl glycogen (Ambion) and left over night at −20° C.

Following centrifugation at maximum speed for 40 min at 4° C., washing with 1 ml Ethanol (85%), and drying, the RNA was re-suspended in 45 μl DDW.

The RNA concentration was tested and DNase Turbo (Ambion) was added accordingly (1 μl DNase/10 μg RNA). Following incubation for 30 min at room temperature and extraction with acid phenol chloroform, the RNA was re-suspend in 45 μl DDW. The RNA concentration was tested again and DNase Turbo (Ambion) was added accordingly (1 μl DNase/10 μg RNA). Following incubation for 30 min at room temperature and extraction with acid phenol chloroform, the RNA was re-suspend in 20 μl DDW.

### 4. Data Normalization

The initial data set was based on signals measured for multiple probes for every sample. For the analysis, signals were used only for probes that were designed to measure the expression levels of known or validated human microRNAs. Data was normalized separately for each group of samples relative to a reference data set, containing one value for each probe. The vector of numbers for the reference data set is represented by R. The reference data set was calculated in one of two ways:

(1) For the expression data (Table 2), each sample type was normalized separately, for example: Lung adenocarcinoma samples. For a given sample type, the value of the reference data set for a given probe was calculated as the median expression of this probe in all samples of the given type.

(2) For the differential expression analysis (data in Table 3), data was normalized separately for each indication relative to a reference data set that was calculated as follows: Each indication consisted of two sample types between which to distinguish, for example: small intestine carcinoid tumor and small intestine stromal tumor. For a given indication, the value of the reference data set for a given probe was calculated as the mean expression of two values: the median expression of this probe in each of the two tumor/tissue types. For example, samples from small intestine carcinoid tumor and small intestine stromal tumor were normalized relative to a reference data set, where the reference value for each probe was the mean expression of the following two values: the median expression of this probe in the all carcinoid tumor samples and the median expression of this probe in all stromal tumor samples.

For each indication (or sample type), the signals of the reference data set (represented by R) were log2 transformed, so that R2=log2(R). For each sample, signals for all probes (represented by S) were log2 transformed, so that S2=log2(S). A second degree polynomial, represented by F, was found for each sample so as to provide the best fit between the sample data and the reference data, such that R2≈F(S2). In this process, remote data points (“outliers”) are not used for calculating the polynomial F. For each probe in the sample, the normalized value V is calculated from the initial value I by transforming it with the polynomial function F, so that V=F(I).

### 5. Statistical Analysis

a. Expression

The purpose of this statistical analysis was to identify microRNAs with expression level significantly higher than the background expression level. The background signal was calculated based on the signal levels of the negative control probes for each sample type. The background level was determined as the 90^{th} percentile of the negative control probes signal values in all samples of the same tissue/tumor. For each probe, we compared the group of normalized signals obtained for a specific tissue/tumor sample set relative to the background signal level. We used a two-sided t-test of the hypothesis that the probe signal levels come from a distribution whose mean is the background signal level. A probe was considered “expressed” if it had a t-test p-value lower than 0.05.

b. Differential Expression

The purpose of this statistical analysis was to find probes whose normalized signal levels differ significantly between the two sample sets being compared. Probes that had normalized signal levels below log2(500) in the two sample sets were not analyzed. For each probe, we compared two groups of normalized signals obtained for two sample sets. For each probe, we calculated the p-value, which is the probability that we would obtain by chance the measured signals or a more extreme difference between the groups if the two groups of signal come from distributions with equal mean values, using the statistical un-paired two-sided t-test method. We selected microRNAs whose probes had t-test p-values lower than 0.05. A p-value lower than 0.05 means that the probability that the two groups come from distributions with the same mean is lower than 0.05 or 5%. The two groups of signal are likely to result from distributions with different means, and the relevant microRNA is likely to be differentially expressed between the two sets of samples.

c. The Probes Types

The probes used are: mirVana™ miRNA Probe Set V2 (Ambion) and other probes designed by Rosetta Genomics. The 3′ amino modifiers of the probes: /3AmMC6/. The chemical structure was as follows:

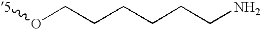

A primary amino group can be used to attach a variety of modifiers (such as fluorescent dyes) to an oligonucleotide or used to attach an oligonucleotide to a solid surface. Amino modifiers can be positioned at the 5′-end with either a standard (C6) or longer (C12) spacer arm. Amino modifications can be positioned at the 3′-end. Internal amino modifications can be introduced using an amino-dT base.

### Example 1

### Specific microRNAs are Expressed in Different Types of Cancers and Normal Tissues

The normalized miRdicator™ arrays mean signals are presented in Table 2. The results show a significant expression pattern of specific miRs in different tumors or normal tissues.

### Example 2

### Specific microRNAs are Able to Distinguish Between Lung Tumor and Normal Lung Tissue

The statistical analysis of the normalized miRdicator™ arrays results of lung tumor versus normal lung tissue are presented in Tables 3-4. The results show a significant difference in the expression pattern of several miRs, most prominent among them being hsa-miR-126 (SEQ ID NO: 204).

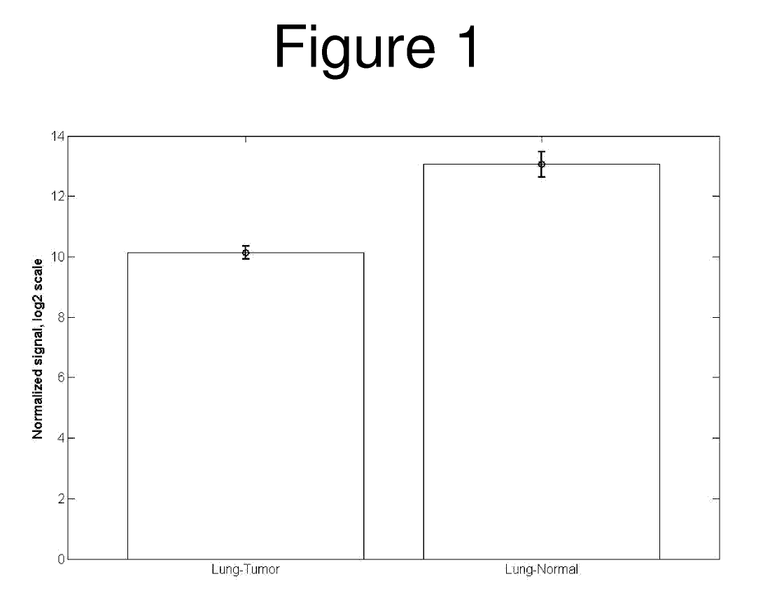

The normalized expression levels of hsa-miR-126 were found to be higher in lung normal tissue in comparison to lung tumor, as measured by miRdicator™ array (FIG. 1). The normalized expression levels of hsa-miR-126 were found to decrease in lung tumors in comparison to normal lung tissue.

### Example 3

### Specific microRNAs are Able to Distinguish Between Liver Metastases and Primary Liver Tumors

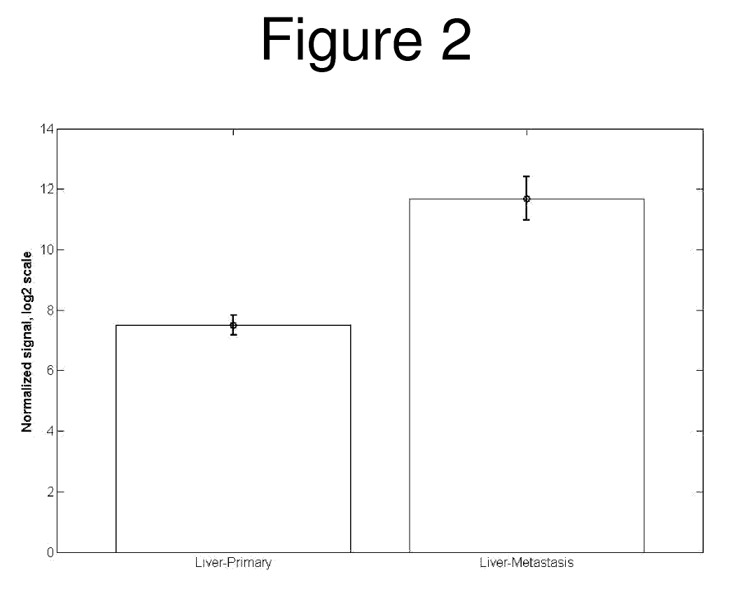

The statistical analysis of the miRdicator™ arrays results of liver metastases vs. primary liver tumors are presented in Tables 3-4. The results show a significant difference in the expression pattern of several miRs, most prominent among them being hsa-miR-200c (SEQ ID NO: 156). The normalized expression levels of hsa-miR-200c were found to increase in liver metastasis in comparison to primary liver tumors (FIG. 2).

### Example 4

### Specific microRNAs are Able to Distinguish Between Small Intestine Carcinoid Tumor and Small Intestine Stromal Tumor

The statistical analysis of the normalized miRdicator™ arrays results of small intestine carcinoid tumor versus small intestine stromal tumor are presented in Tables 3-4. The results show a significant difference in the expression pattern of several miRs, most prominent among them being hsa-miR-375 (SEQ ID NO: 273).

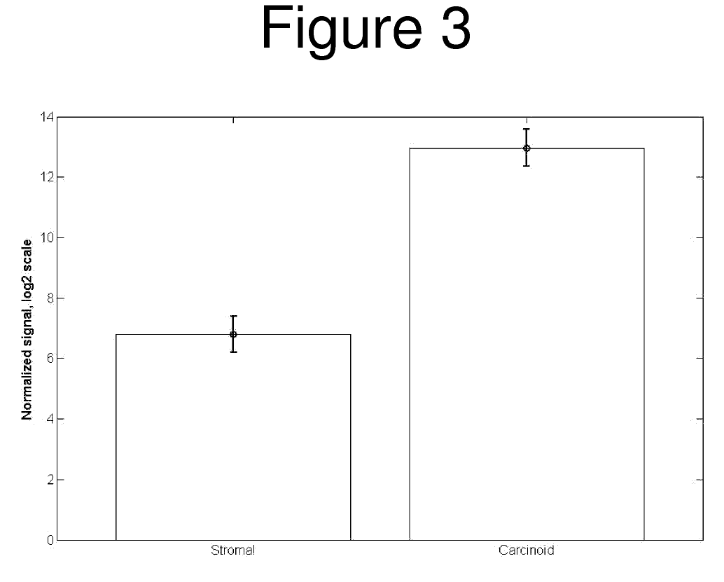

The normalized expression levels of hsa-miR-375 were found to be higher in small intestine carcinoid tumors in comparison to small intestine stromal tumors, as measured by miRdicator™ array (FIG. 3).

### Example 5

### Specific microRNAs are Able to Distinguish Between Different Grades of Bladder Carcinoma

The statistical analysis of the miRdicator™ arrays results of high grade bladder carcinoma versus medium grade bladder carcinoma or low grade bladder carcinoma are presented in Tables 3-4. The results exhibited a significant difference in the expression pattern of 43_{—}5 (SEQ ID NO: 215). For Table 4, “DID” is the Decision ID, describing the comparisons of one group of tissue(s) to another group of tissue(s).

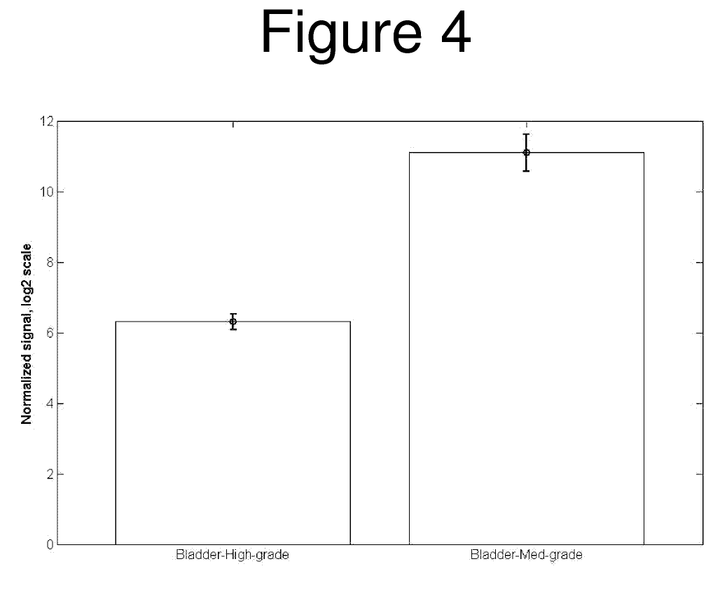

The normalized expression levels of 43_{—}5 were found to increase in medium grade bladder carcinoma in comparison to high grade bladder carcinoma, as measured by miRdicator™ array (FIG. 4).

TABLE 1

| TID | Tissue_name |
|---|---|
| 1 | Bladder-Normal |
| 2 | Bladder-Transient Carcinoma |
| 3 | Bladder-Tumor |
| 4 | Bladder-Tumor-High-Grade |
| 5 | Bladder-Tumor-Low-Grade |
| 6 | Bladder-Tumor-Med-Grade |
| 7 | Breast-Normal |
| 8 | Breast-Tumor |
| 9 | Breast-Tumor-High-Grade |
| 10 | Breast-Tumor-Med-Grade |
| 11 | Cervical-Tumor |
| 12 | Colon-Normal |
| 13 | Colon-Tumor |
| 14 | Colon-Tumor-Low-Grade |
| 15 | Colon-Tumor-Med-Grade |
| 16 | Esophageal-Tumor |
| 17 | Gallbladder-Tumor |
| 18 | Kidney-Normal |
| 19 | Kidney-Tumor |
| 20 | Liver-Metast. |
| 21 | Liver-Normal |
| 22 | Liver-Tumor |
| 23 | Lung-Adeno |
| 24 | Lung-Large-cell |
| 25 | Lung-Normal |
| 26 | Lung-Squamous |
| 27 | Lung-Tumor |
| 28 | Lung-Tumor-High-Grade |
| 29 | Lung-Tumor-Low-Grade |
| 30 | Lung-Tumor-Med-Grade |
| 31 | Melanoma-Lymph Node-Metast. |
| 32 | Ovarian-Normal |
| 33 | Ovarian-Tumor |
| 34 | Prostate-Adeno |
| 35 | Prostate-BPH |
| 36 | Prostate-Normal |
| 37 | Prostate-Tumor |
| 38 | Prostate-Tumor-High-Grade |
| 39 | Prostate-Tumor-Low-Grade |
| 40 | Prostate-Tumor-Med-Grade |
| 41 | Small intestine-Carcinoid |
| 42 | Small intestine-Normal |
| 43 | Small intestine-Stromal |
| 44 | Small intestine-Tumor |
| 45 | Small intestine-Tumor-Med-Grade |
| 46 | Stomach-Tumor |
| 47 | Testicular-Tumor |
| 48 | Thyroid-Normal |
| 49 | Thyroid-Tumor |
| 50 | Uterus-Normal |
| 51 | Uterus-Tumor |

TABLE 2

| RID | TID | MID | HIDs | mirname | signal | n | p_value |
|---|---|---|---|---|---|---|---|
| 1 | 1 | 127 | 361 | 11_1 | 523 | 14 | 1.81e−032 |
| 2 | 1 | 196 | 508 | 15_rel | 60425 | 3 | 1.26e−006 |
| 3 | 1 | 219 | 497 | 27_3 | 706 | 14 | 3.1e−037 |
| 4 | 1 | 94 | 394 | 69_6_rel | 15569 | 3 | 4.58e−006 |
| 5 | 1 | 177 | 366, 464 | 72_12 | 873 | 3 | 2.77e−006 |
| 6 | 1 | 40 | 553 | 72_4 | 749 | 3 | 2.3e−006 |
| 7 | 1 | 97 | 357 | 74_26_rel | 1979 | 3 | 8.55e−006 |
| 8 | 1 | 163 | 402 | 43_7 | 520 | 3 | 2.47e−006 |
| 9 | 1 | 223 | 320, 393, 559 | hsa-let-7a | 7730 | 14 | 1.16e−033 |
| 10 | 1 | 225 | 332 | hsa-let-7b | 9773 | 14 | 3.62e−037 |
| 11 | 1 | 224 | 427 | hsa-let-7c | 10860 | 14 | 3.44e−037 |
| 12 | 1 | 45 | 277 | hsa-let-7d | 2327 | 14 | 1.16e−037 |
| 13 | 1 | 220 | 475 | hsa-let-7e | 3616 | 14 | 2.15e−038 |
| 14 | 1 | 222 | 482, 558 | hsa-let-7f | 625 | 14 | 7.46e−034 |
| 15 | 1 | 226 | 395 | hsa-let-7g | 783 | 14 | 2.63e−038 |
| 16 | 1 | 227 | 297 | hsa-let-7i | 1509 | 14 | 2.1e−036 |
| 17 | 1 | 13 | 397 | hsa-miR-100 | 2303 | 14 | 1.52e−037 |
| 18 | 1 | 48 | 483, 529 | hsa-miR-103 | 1381 | 14 | 2.47e−036 |
| 19 | 1 | 184 | 423 | hsa-miR-105 | 629 | 14 | 9.95e−035 |
| 20 | 1 | 2 | 399 | hsa-miR-106a | 801 | 14 | 0 |
| 21 | 1 | 149 | 392 | hsa-miR-106b | 650 | 14 | 7.18e−036 |
| 22 | 1 | 47 | 555 | hsa-miR-107 | 1353 | 14 | 2.98e−037 |
| 23 | 1 | 199 | 387 | hsa-miR-125a | 3982 | 14 | 1.37e−033 |
| 24 | 1 | 198 | 313, 551 | hsa-miR-125b | 17861 | 14 | 3.52e−035 |
| 25 | 1 | 204 | 419 | hsa-miR-126 | 3300 | 14 | 2.61e−034 |
| 26 | 1 | 86 | 417 | hsa-miR-130a | 1618 | 14 | 0 |
| 27 | 1 | 266 | 295, 386 | hsa-miR-133a | 1610 | 14 | 3.73e−035 |
| 28 | 1 | 265 | 533 | hsa-miR-133b | 582 | 3 | 1.59e−006 |
| 29 | 1 | 151 | 472 | hsa-miR-141 | 633 | 14 | 4.89e−033 |
| 30 | 1 | 218 | 527 | hsa-miR-143 | 48542 | 14 | 0 |
| 31 | 1 | 139 | 281 | hsa-miR-145 | 44647 | 14 | 0 |
| 32 | 1 | 192 | 470 | hsa-miR-152 | 510 | 14 | 0 |
| 33 | 1 | 172 | 329 | hsa-miR-15b | 708 | 14 | 7.24e−033 |
| 34 | 1 | 173 | 360, 468 | hsa-miR-16 | 3582 | 14 | 1.66e−035 |
| 35 | 1 | 75 | 467 | hsa-miR-17-5p | 568 | 14 | 1.41e−035 |
| 36 | 1 | 10 | 371, 461 | hsa-miR-181a | 774 | 14 | 0 |
| 37 | 1 | 234 | 450 | hsa-miR-185 | 513 | 14 | 0 |
| 38 | 1 | 77 | 440 | hsa-miR-191 | 1078 | 14 | 0 |
| 39 | 1 | 17 | 442 | hsa-miR-193a | 3289 | 14 | 2.21e−036 |
| 40 | 1 | 16 | 488 | hsa-miR-193b | 551 | 14 | 9.36e−032 |
| 41 | 1 | 170 | 327 | hsa-miR-195 | 1422 | 14 | 2.88e−035 |
| 42 | 1 | 92 | 410, 432 | hsa-miR-199a | 4820 | 14 | 0 |
| 43 | 1 | 162 | 410, 432, 473 | hsa-miR-199a* | 1726 | 14 | 1.01e−034 |
| 44 | 1 | 251 | 282, 495 | hsa-miR-19b | 886 | 14 | 0 |
| 45 | 1 | 157 | 411 | hsa-miR-200b | 529 | 14 | 1.1e−034 |
| 46 | 1 | 156 | 446 | hsa-miR-200c | 906 | 14 | 2.13e−034 |
| 47 | 1 | 203 | 504 | hsa-miR-205 | 2074 | 14 | 6.09e−034 |
| 48 | 1 | 175 | 462 | hsa-miR-21 | 7793 | 14 | 6.28e−034 |
| 49 | 1 | 29 | 439 | hsa-miR-214 | 1716 | 14 | 2.25e−037 |
| 50 | 1 | 18 | 322 | hsa-miR-22 | 3877 | 14 | 9.88e−037 |
| 51 | 1 | 50 | 364 | hsa-miR-221 | 3175 | 14 | 0 |
| 52 | 1 | 49 | 302 | hsa-miR-222 | 4160 | 14 | 1.37e−038 |
| 53 | 1 | 67 | 336 | hsa-miR-23a | 6821 | 14 | 2.18e−035 |
| 54 | 1 | 66 | 306 | hsa-miR-23b | 6911 | 14 | 1.18e−027 |
| 55 | 1 | 238 | 346, 416 | hsa-miR-24 | 12982 | 14 | 0 |
| 56 | 1 | 256 | 441, 487 | hsa-miR-26a | 8771 | 14 | 2.02e−037 |
| 57 | 1 | 259 | 481 | hsa-miR-27a | 7345 | 14 | 5.78e−037 |
| 58 | 1 | 260 | 280 | hsa-miR-27b | 4987 | 14 | 1.5e−034 |
| 59 | 1 | 61 | 286 | hsa-miR-296 | 1028 | 14 | 1.58e−034 |
| 60 | 1 | 167 | 556 | hsa-miR-29a | 1604 | 14 | 2.75e−029 |
| 61 | 1 | 244 | 287 | hsa-miR-30a-5p | 1072 | 14 | 2.94e−030 |
| 62 | 1 | 242 | 311 | hsa-miR-30b | 741 | 14 | 5.33e−033 |
| 63 | 1 | 243 | 299, 316 | hsa-miR-30c | 1509 | 14 | 1.33e−034 |
| 64 | 1 | 241 | 550 | hsa-miR-30d | 889 | 14 | 1.78e−031 |
| 65 | 1 | 245 | 431 | hsa-miR-30e-5p | 1456 | 14 | 1.44e−037 |
| 66 | 1 | 132 | 429 | hsa-miR-31 | 692 | 14 | 1.51e−033 |
| 67 | 1 | 1 | 359 | hsa-miR-320 | 2535 | 14 | 1.38e−036 |
| 68 | 1 | 91 | 368 | hsa-miR-324-3p | 522 | 14 | 0 |
| 69 | 1 | 126 | 380 | hsa-miR-331 | 791 | 14 | 4.12e−035 |
| 70 | 1 | 208 | 477 | hsa-miR-342 | 606 | 14 | 0 |
| 71 | 1 | 237 | 480 | hsa-miR-34a | 1440 | 14 | 0 |
| 72 | 1 | 112 | 372 | hsa-miR-422b | 896 | 14 | 3.78e−035 |
| 73 | 1 | 51 | 497 | hsa-miR-423 | 1338 | 14 | 5.37e−037 |
| 74 | 1 | 63 | 557 | hsa-miR-491 | 921 | 14 | 1.22e−032 |
| 75 | 1 | 214 | 376 | hsa-miR-494 | 5173 | 14 | 3.49e−034 |
| 76 | 1 | 83 | 312 | hsa-miR-497 | 2027 | 14 | 1.03e−036 |
| 77 | 1 | 135 | 315 | hsa-miR-565 | 672 | 3 | 1.08e−006 |
| 78 | 1 | 60 | 479 | hsa-miR-638 | 23074 | 3 | 5.78e−006 |
| 79 | 1 | 182 | 304, 317 | hsa-miR-92 | 1349 | 14 | 6.11e−037 |
| 80 | 1 | 6 | 471 | hsa-miR-93 | 788 | 14 | 1.45e−038 |
| 81 | 1 | 14 | 318 | hsa-miR-99a | 3586 | 14 | 1.1e−036 |
| 82 | 1 | 80 | 509 | hsa-miR-99b | 760 | 14 | 2.41e−034 |
| 83 | 1 | 131 | 430 | 6_11 | 554 | 14 | 2.84e−034 |
| 84 | 2 | 127 | 361 | 11_1 | 929 | 35 | 0 |
| 85 | 2 | 196 | 508 | 15_rel | 19536 | 32 | 0 |
| 86 | 2 | 219 | 497 | 27_3 | 1278 | 35 | 0 |
| 87 | 2 | 129 | 548 | 43_39 | 664 | 35 | 0 |
| 88 | 2 | 145 | 447 | 43_44_rel | 53789 | 5 | 4.05e−013 |
| 89 | 2 | 94 | 394 | 69_6_rel | 5165 | 32 | 0 |
| 90 | 2 | 177 | 366, 464 | 72_12 | 1607 | 32 | 0 |
| 91 | 2 | 40 | 553 | 72_4 | 1234 | 32 | 0 |
| 92 | 2 | 97 | 357 | 74_26_rel | 2105 | 32 | 0 |
| 93 | 2 | 33 | 339, 552 | 96_rel | 32675 | 5 | 6.52e−013 |
| 94 | 2 | 87 | 301 | gam11 | 1076 | 32 | 0 |
| 95 | 2 | 163 | 402 | 43_7 | 507 | 32 | 0 |
| 96 | 2 | 223 | 320, 393, 559 | hsa-let-7a | 3458 | 35 | 0 |
| 97 | 2 | 223 | 320, 393, 559 | hsa-let-7a | 831 | 10 | 2.92e−023 |
| 98 | 2 | 225 | 332 | hsa-let-7b | 9176 | 35 | 0 |
| 99 | 2 | 224 | 427 | hsa-let-7c | 4936 | 35 | 0 |
| 100 | 2 | 45 | 277 | hsa-let-7d | 2211 | 35 | 0 |
| 101 | 2 | 220 | 475 | hsa-let-7e | 2782 | 35 | 0 |
| 102 | 2 | 222 | 482, 558 | hsa-let-7f | 510 | 35 | 0 |
| 103 | 2 | 226 | 395 | hsa-let-7g | 1158 | 35 | 0 |
| 104 | 2 | 227 | 297 | hsa-let-7i | 1327 | 35 | 0 |
| 105 | 2 | 13 | 397 | hsa-miR-100 | 824 | 35 | 0 |
| 106 | 2 | 48 | 483, 529 | hsa-miR-103 | 2449 | 35 | 0 |
| 107 | 2 | 184 | 423 | hsa-miR-105 | 1152 | 35 | 0 |
| 108 | 2 | 2 | 399 | hsa-miR-106a | 2244 | 35 | 0 |
| 109 | 2 | 149 | 392 | hsa-miR-106b | 1770 | 35 | 0 |
| 110 | 2 | 47 | 555 | hsa-miR-107 | 2897 | 35 | 0 |
| 111 | 2 | 199 | 387 | hsa-miR-125a | 1769 | 35 | 0 |
| 112 | 2 | 198 | 313, 551 | hsa-miR-125b | 3108 | 35 | 0 |
| 113 | 2 | 204 | 419 | hsa-miR-126 | 1677 | 35 | 0 |
| 114 | 2 | 86 | 417 | hsa-miR-130a | 1794 | 35 | 0 |
| 115 | 2 | 85 | 328 | hsa-miR-130b | 546 | 35 | 0 |
| 116 | 2 | 64 | 402 | hsa-miR-140 | 548 | 35 | 0 |
| 117 | 2 | 151 | 472 | hsa-miR-141 | 5569 | 35 | 0 |
| 118 | 2 | 218 | 527 | hsa-miR-143 | 2733 | 35 | 0 |
| 119 | 2 | 139 | 281 | hsa-miR-145 | 5344 | 35 | 0 |
| 120 | 2 | 191 | 289 | hsa-miR-148b | 519 | 35 | 0 |
| 121 | 2 | 212 | 333 | hsa-miR-149 | 798 | 35 | 0 |
| 122 | 2 | 209 | 507 | hsa-miR-150 | 525 | 35 | 0 |
| 123 | 2 | 34 | 396 | hsa-miR-151 | 737 | 35 | 0 |
| 124 | 2 | 172 | 329 | hsa-miR-15b | 794 | 35 | 0 |
| 125 | 2 | 173 | 360, 468 | hsa-miR-16 | 3442 | 35 | 0 |
| 126 | 2 | 38 | 467 | hsa-miR-17-3p | 585 | 35 | 0 |
| 127 | 2 | 75 | 467 | hsa-miR-17-5p | 1704 | 35 | 0 |
| 128 | 2 | 10 | 371, 461 | hsa-miR-181a | 1683 | 35 | 0 |
| 129 | 2 | 11 | 456, 540 | hsa-miR-181b | 635 | 35 | 0 |
| 130 | 2 | 272 | 323 | hsa-miR-182 | 526 | 35 | 0 |
| 131 | 2 | 179 | 356 | hsa-miR-183 | 669 | 35 | 0 |
| 132 | 2 | 234 | 450 | hsa-miR-185 | 648 | 35 | 0 |
| 133 | 2 | 155 | 426 | hsa-miR-18a | 502 | 35 | 0 |
| 134 | 2 | 77 | 440 | hsa-miR-191 | 2986 | 35 | 0 |
| 135 | 2 | 17 | 442 | hsa-miR-193a | 1414 | 35 | 0 |
| 136 | 2 | 16 | 488 | hsa-miR-193b | 530 | 35 | 0 |
| 137 | 2 | 92 | 410, 432 | hsa-miR-199a | 2399 | 35 | 0 |
| 138 | 2 | 162 | 410, 432, 473 | hsa-miR-199a* | 516 | 35 | 0 |
| 139 | 2 | 251 | 282, 495 | hsa-miR-19b | 1132 | 35 | 0 |
| 140 | 2 | 152 | 369 | hsa-miR-200a | 1075 | 35 | 0 |
| 141 | 2 | 157 | 411 | hsa-miR-200b | 2453 | 35 | 0 |
| 142 | 2 | 156 | 446 | hsa-miR-200c | 6170 | 35 | 0 |
| 143 | 2 | 203 | 504 | hsa-miR-205 | 10873 | 35 | 0 |
| 144 | 2 | 175 | 462 | hsa-miR-21 | 5697 | 35 | 0 |
| 145 | 2 | 115 | 314 | hsa-miR-210 | 1102 | 35 | 0 |
| 146 | 2 | 29 | 439 | hsa-miR-214 | 1123 | 35 | 0 |
| 147 | 2 | 18 | 322 | hsa-miR-22 | 3006 | 35 | 0 |
| 148 | 2 | 50 | 364 | hsa-miR-221 | 2040 | 35 | 0 |
| 149 | 2 | 49 | 302 | hsa-miR-222 | 2319 | 35 | 0 |
| 150 | 2 | 67 | 336 | hsa-miR-23a | 3859 | 35 | 0 |
| 151 | 2 | 66 | 306 | hsa-miR-23b | 628 | 35 | 0 |
| 152 | 2 | 238 | 346, 416 | hsa-miR-24 | 8689 | 35 | 0 |
| 153 | 2 | 256 | 441, 487 | hsa-miR-26a | 5350 | 35 | 0 |
| 154 | 2 | 259 | 481 | hsa-miR-27a | 4072 | 35 | 0 |
| 155 | 2 | 260 | 280 | hsa-miR-27b | 1668 | 35 | 0 |
| 156 | 2 | 61 | 286 | hsa-miR-296 | 708 | 35 | 0 |
| 157 | 2 | 242 | 311 | hsa-miR-30b | 577 | 35 | 0 |
| 158 | 2 | 243 | 299, 316 | hsa-miR-30c | 912 | 35 | 0 |
| 159 | 2 | 241 | 550 | hsa-miR-30d | 1655 | 35 | 0 |
| 160 | 2 | 245 | 431 | hsa-miR-30e-5p | 1744 | 35 | 0 |
| 161 | 2 | 132 | 429 | hsa-miR-31 | 2031 | 35 | 0 |
| 162 | 2 | 1 | 359 | hsa-miR-320 | 4085 | 35 | 0 |
| 163 | 2 | 91 | 368 | hsa-miR-324-3p | 751 | 35 | 0 |
| 164 | 2 | 98 | 368 | hsa-miR-324-5p | 570 | 35 | 0 |
| 165 | 2 | 113 | 538 | hsa-miR-328 | 629 | 35 | 0 |
| 166 | 2 | 141 | 437 | hsa-miR-33 | 531 | 35 | 0 |
| 167 | 2 | 126 | 380 | hsa-miR-331 | 735 | 35 | 0 |
| 168 | 2 | 208 | 477 | hsa-miR-342 | 813 | 35 | 0 |
| 169 | 2 | 229 | 292 | hsa-miR-345 | 680 | 35 | 0 |
| 170 | 2 | 237 | 480 | hsa-miR-34a | 2562 | 35 | 0 |
| 171 | 2 | 160 | 379, 382 | hsa-miR-365 | 508 | 35 | 0 |
| 172 | 2 | 128 | 294 | hsa-miR-370 | 707 | 35 | 0 |
| 173 | 2 | 111 | 389 | hsa-miR-422a | 536 | 35 | 0 |
| 174 | 2 | 112 | 372 | hsa-miR-422b | 1299 | 35 | 0 |
| 175 | 2 | 51 | 497 | hsa-miR-423 | 2239 | 35 | 0 |
| 176 | 2 | 158 | 378 | hsa-miR-429 | 522 | 35 | 0 |
| 177 | 2 | 236 | 413 | hsa-miR-449 | 1412 | 35 | 0 |
| 178 | 2 | 252 | 405 | hsa-miR-452 | 555 | 35 | 0 |
| 179 | 2 | 63 | 557 | hsa-miR-491 | 2306 | 35 | 0 |
| 180 | 2 | 54 | 293 | hsa-miR-492 | 584 | 35 | 0 |
| 181 | 2 | 214 | 376 | hsa-miR-494 | 4882 | 35 | 0 |
| 182 | 2 | 83 | 312 | hsa-miR-497 | 787 | 35 | 0 |
| 183 | 2 | 43 | 418 | hsa-miR-504 | 667 | 35 | 0 |
| 184 | 2 | 258 | 428, 443 | hsa-miR-513 | 639 | 35 | 0 |
| 185 | 2 | 210 | 400 | hsa-miR-518c* | 530 | 35 | 0 |
| 186 | 2 | 135 | 315 | hsa-miR-565 | 2060 | 32 | 0 |
| 187 | 2 | 60 | 479 | hsa-miR-638 | 8564 | 32 | 0 |
| 188 | 2 | 123 | 291 | hsa-miR-801 | 1759 | 10 | 3.63e−024 |
| 189 | 2 | 182 | 304, 317 | hsa-miR-92 | 2663 | 35 | 0 |
| 190 | 2 | 6 | 471 | hsa-miR-93 | 2777 | 35 | 0 |
| 191 | 2 | 14 | 318 | hsa-miR-99a | 1151 | 35 | 0 |
| 192 | 2 | 80 | 509 | hsa-miR-99b | 1129 | 35 | 0 |
| 193 | 2 | 131 | 430 | 6_11 | 654 | 35 | 0 |
| 194 | 3 | 127 | 361 | 11_1 | 951 | 41 | 0 |
| 195 | 3 | 196 | 508 | 15_rel | 18649 | 37 | 0 |
| 196 | 3 | 219 | 497 | 27_3 | 1304 | 41 | 0 |
| 197 | 3 | 129 | 548 | 43_39 | 664 | 41 | 0 |
| 198 | 3 | 145 | 447 | 43_44_rel | 52893 | 5 | 5.72e−013 |
| 199 | 3 | 94 | 394 | 69_6_rel | 5034 | 37 | 0 |
| 200 | 3 | 177 | 366, 464 | 72_12 | 1497 | 37 | 0 |
| 201 | 3 | 40 | 553 | 72_4 | 1194 | 37 | 0 |
| 202 | 3 | 97 | 357 | 74_26_rel | 2043 | 37 | 0 |
| 203 | 3 | 33 | 339, 552 | 96_rel | 32258 | 5 | 7.5e−013 |
| 204 | 3 | 87 | 301 | gam11 | 1165 | 37 | 0 |
| 205 | 3 | 163 | 402 | 43_7 | 528 | 37 | 0 |
| 206 | 3 | 223 | 320, 393, 559 | hsa-let-7a | 3586 | 41 | 0 |
| 207 | 3 | 223 | 320, 393, 559 | hsa-let-7a | 995 | 12 | 5.79e−027 |
| 208 | 3 | 225 | 332 | hsa-let-7b | 9305 | 41 | 0 |
| 209 | 3 | 224 | 427 | hsa-let-7c | 5199 | 41 | 0 |
| 210 | 3 | 45 | 277 | hsa-let-7d | 2260 | 41 | 0 |
| 211 | 3 | 220 | 475 | hsa-let-7e | 2859 | 41 | 0 |
| 212 | 3 | 222 | 482, 558 | hsa-let-7f | 525 | 41 | 0 |
| 213 | 3 | 226 | 395 | hsa-let-7g | 1125 | 41 | 0 |
| 214 | 3 | 227 | 297 | hsa-let-7i | 1328 | 41 | 0 |
| 215 | 3 | 13 | 397 | hsa-miR-100 | 911 | 41 | 0 |
| 216 | 3 | 48 | 483, 529 | hsa-miR-103 | 2550 | 41 | 0 |
| 217 | 3 | 184 | 423 | hsa-miR-105 | 1152 | 41 | 0 |
| 218 | 3 | 2 | 399 | hsa-miR-106a | 2099 | 41 | 0 |
| 219 | 3 | 149 | 392 | hsa-miR-106b | 1707 | 41 | 0 |
| 220 | 3 | 47 | 555 | hsa-miR-107 | 2882 | 41 | 0 |
| 221 | 3 | 199 | 387 | hsa-miR-125a | 1777 | 41 | 0 |
| 222 | 3 | 198 | 313, 551 | hsa-miR-125b | 3252 | 41 | 0 |
| 223 | 3 | 204 | 419 | hsa-miR-126 | 1725 | 41 | 0 |
| 224 | 3 | 86 | 417 | hsa-miR-130a | 1789 | 41 | 0 |
| 225 | 3 | 85 | 328 | hsa-miR-130b | 524 | 41 | 0 |
| 226 | 3 | 64 | 402 | hsa-miR-140 | 531 | 41 | 0 |
| 227 | 3 | 151 | 472 | hsa-miR-141 | 4351 | 41 | 0 |
| 228 | 3 | 218 | 527 | hsa-miR-143 | 2944 | 41 | 0 |
| 229 | 3 | 139 | 281 | hsa-miR-145 | 5804 | 41 | 0 |
| 230 | 3 | 191 | 289 | hsa-miR-148b | 505 | 41 | 0 |
| 231 | 3 | 212 | 333 | hsa-miR-149 | 791 | 41 | 0 |
| 232 | 3 | 209 | 507 | hsa-miR-150 | 522 | 41 | 0 |
| 233 | 3 | 34 | 396 | hsa-miR-151 | 720 | 41 | 0 |
| 234 | 3 | 172 | 329 | hsa-miR-15b | 811 | 41 | 0 |
| 235 | 3 | 173 | 360, 468 | hsa-miR-16 | 3512 | 41 | 0 |
| 236 | 3 | 38 | 467 | hsa-miR-17-3p | 576 | 41 | 0 |
| 237 | 3 | 75 | 467 | hsa-miR-17-5p | 1628 | 41 | 0 |
| 238 | 3 | 10 | 371, 461 | hsa-miR-181a | 1728 | 41 | 0 |
| 239 | 3 | 11 | 456, 540 | hsa-miR-181b | 664 | 41 | 0 |
| 240 | 3 | 179 | 356 | hsa-miR-183 | 623 | 41 | 0 |
| 241 | 3 | 234 | 450 | hsa-miR-185 | 674 | 41 | 0 |
| 242 | 3 | 77 | 440 | hsa-miR-191 | 2854 | 41 | 0 |
| 243 | 3 | 17 | 442 | hsa-miR-193a | 1433 | 41 | 0 |
| 244 | 3 | 16 | 488 | hsa-miR-193b | 561 | 41 | 0 |
| 245 | 3 | 92 | 410, 432 | hsa-miR-199a | 2518 | 41 | 0 |
| 246 | 3 | 162 | 410, 432, 473 | hsa-miR-199a* | 562 | 41 | 0 |
| 247 | 3 | 251 | 282, 495 | hsa-miR-19b | 1122 | 41 | 0 |
| 248 | 3 | 152 | 369 | hsa-miR-200a | 1066 | 41 | 0 |
| 249 | 3 | 157 | 411 | hsa-miR-200b | 2097 | 41 | 0 |
| 250 | 3 | 156 | 446 | hsa-miR-200c | 4990 | 41 | 0 |
| 251 | 3 | 203 | 504 | hsa-miR-205 | 9215 | 41 | 0 |
| 252 | 3 | 175 | 462 | hsa-miR-21 | 5284 | 41 | 0 |
| 253 | 3 | 115 | 314 | hsa-miR-210 | 1080 | 41 | 0 |
| 254 | 3 | 29 | 439 | hsa-miR-214 | 1148 | 41 | 0 |
| 255 | 3 | 18 | 322 | hsa-miR-22 | 3035 | 41 | 0 |
| 256 | 3 | 50 | 364 | hsa-miR-221 | 2161 | 41 | 0 |
| 257 | 3 | 49 | 302 | hsa-miR-222 | 2403 | 41 | 0 |
| 258 | 3 | 67 | 336 | hsa-miR-23a | 3951 | 41 | 0 |
| 259 | 3 | 66 | 306 | hsa-miR-23b | 725 | 41 | 0 |
| 260 | 3 | 238 | 346, 416 | hsa-miR-24 | 9079 | 41 | 0 |
| 261 | 3 | 256 | 441, 487 | hsa-miR-26a | 5448 | 41 | 0 |
| 262 | 3 | 259 | 481 | hsa-miR-27a | 4164 | 41 | 0 |
| 263 | 3 | 260 | 280 | hsa-miR-27b | 1780 | 41 | 0 |
| 264 | 3 | 61 | 286 | hsa-miR-296 | 708 | 41 | 0 |
| 265 | 3 | 242 | 311 | hsa-miR-30b | 570 | 41 | 0 |
| 266 | 3 | 243 | 299, 316 | hsa-miR-30c | 978 | 41 | 0 |
| 267 | 3 | 241 | 550 | hsa-miR-30d | 1601 | 41 | 0 |
| 268 | 3 | 245 | 431 | hsa-miR-30e-5p | 1764 | 41 | 0 |
| 269 | 3 | 132 | 429 | hsa-miR-31 | 2000 | 41 | 0 |
| 270 | 3 | 1 | 359 | hsa-miR-320 | 4060 | 41 | 0 |
| 271 | 3 | 91 | 368 | hsa-miR-324-3p | 750 | 41 | 0 |
| 272 | 3 | 98 | 368 | hsa-miR-324-5p | 571 | 41 | 0 |
| 273 | 3 | 113 | 538 | hsa-miR-328 | 627 | 41 | 0 |
| 274 | 3 | 126 | 380 | hsa-miR-331 | 737 | 41 | 0 |
| 275 | 3 | 208 | 477 | hsa-miR-342 | 837 | 41 | 0 |
| 276 | 3 | 229 | 292 | hsa-miR-345 | 660 | 41 | 0 |
| 277 | 3 | 237 | 480 | hsa-miR-34a | 2574 | 41 | 0 |
| 278 | 3 | 160 | 379, 382 | hsa-miR-365 | 508 | 41 | 0 |
| 279 | 3 | 128 | 294 | hsa-miR-370 | 712 | 41 | 0 |
| 280 | 3 | 111 | 389 | hsa-miR-422a | 522 | 41 | 0 |
| 281 | 3 | 112 | 372 | hsa-miR-422b | 1327 | 41 | 0 |
| 282 | 3 | 51 | 497 | hsa-miR-423 | 2159 | 41 | 0 |
| 283 | 3 | 236 | 413 | hsa-miR-449 | 1404 | 41 | 0 |
| 284 | 3 | 252 | 405 | hsa-miR-452 | 536 | 41 | 0 |
| 285 | 3 | 63 | 557 | hsa-miR-491 | 2177 | 41 | 0 |
| 286 | 3 | 54 | 293 | hsa-miR-492 | 565 | 41 | 0 |
| 287 | 3 | 214 | 376 | hsa-miR-494 | 4444 | 41 | 0 |
| 288 | 3 | 83 | 312 | hsa-miR-497 | 852 | 41 | 0 |
| 289 | 3 | 43 | 418 | hsa-miR-504 | 670 | 41 | 0 |
| 290 | 3 | 258 | 428, 443 | hsa-miR-513 | 635 | 41 | 0 |
| 291 | 3 | 210 | 400 | hsa-miR-518c* | 521 | 41 | 0 |
| 292 | 3 | 135 | 315 | hsa-miR-565 | 2159 | 37 | 0 |
| 293 | 3 | 60 | 479 | hsa-miR-638 | 8235 | 37 | 0 |
| 294 | 3 | 123 | 291 | hsa-miR-801 | 1757 | 12 | 1.89e−028 |
| 295 | 3 | 182 | 304, 317 | hsa-miR-92 | 2597 | 41 | 0 |
| 296 | 3 | 6 | 471 | hsa-miR-93 | 2663 | 41 | 0 |
| 297 | 3 | 14 | 318 | hsa-miR-99a | 1256 | 41 | 0 |
| 298 | 3 | 80 | 509 | hsa-miR-99b | 1147 | 41 | 0 |
| 299 | 3 | 131 | 430 | 6_11 | 662 | 41 | 0 |
| 300 | 4 | 127 | 361 | 11_1 | 610 | 21 | 0 |
| 301 | 4 | 196 | 508 | 15_rel | 32688 | 19 | 0 |
| 302 | 4 | 219 | 497 | 27_3 | 1462 | 21 | 0 |
| 303 | 4 | 129 | 548 | 43_39 | 703 | 21 | 0 |
| 304 | 4 | 145 | 447 | 43_44_rel | 50499 | 4 | 6.89e−010 |
| 305 | 4 | 183 | 373 | 66_1 | 536 | 21 | 0 |
| 306 | 4 | 94 | 394 | 69_6_rel | 7221 | 19 | 0 |
| 307 | 4 | 177 | 366, 464 | 72_12 | 1167 | 19 | 0 |
| 308 | 4 | 40 | 553 | 72_4 | 633 | 19 | 0 |
| 309 | 4 | 97 | 357 | 74_26_rel | 1944 | 19 | 0 |
| 310 | 4 | 33 | 339, 552 | 96_rel | 29828 | 4 | 1.51e−009 |
| 311 | 4 | 87 | 301 | gam11 | 1739 | 19 | 4.08e−034 |
| 312 | 4 | 223 | 320, 393, 559 | hsa-let-7a | 2717 | 21 | 0 |
| 313 | 4 | 223 | 320, 393, 559 | hsa-let-7a | 854 | 9 | 1.46e−019 |
| 314 | 4 | 225 | 332 | hsa-let-7b | 7445 | 21 | 0 |
| 315 | 4 | 224 | 427 | hsa-let-7c | 4442 | 21 | 0 |
| 316 | 4 | 45 | 277 | hsa-let-7d | 1733 | 21 | 0 |
| 317 | 4 | 220 | 475 | hsa-let-7e | 2532 | 21 | 0 |
| 318 | 4 | 226 | 395 | hsa-let-7g | 970 | 21 | 0 |
| 319 | 4 | 227 | 297 | hsa-let-7i | 1165 | 21 | 0 |
| 320 | 4 | 13 | 397 | hsa-miR-100 | 1194 | 21 | 0 |
| 321 | 4 | 48 | 483, 529 | hsa-miR-103 | 2280 | 21 | 0 |
| 322 | 4 | 184 | 423 | hsa-miR-105 | 946 | 21 | 0 |
| 323 | 4 | 2 | 399 | hsa-miR-106a | 1621 | 21 | 0 |
| 324 | 4 | 149 | 392 | hsa-miR-106b | 1356 | 21 | 0 |
| 325 | 4 | 47 | 555 | hsa-miR-107 | 1992 | 21 | 0 |
| 326 | 4 | 199 | 387 | hsa-miR-125a | 1568 | 21 | 0 |
| 327 | 4 | 198 | 313, 551 | hsa-miR-125b | 5002 | 21 | 0 |
| 328 | 4 | 204 | 419 | hsa-miR-126 | 1281 | 21 | 0 |
| 329 | 4 | 89 | 419 | hsa-miR-126* | 567 | 21 | 0 |
| 330 | 4 | 86 | 417 | hsa-miR-130a | 1751 | 21 | 0 |
| 331 | 4 | 52 | 370, 548 | hsa-miR-138 | 515 | 21 | 0 |
| 332 | 4 | 64 | 402 | hsa-miR-140 | 526 | 21 | 0 |
| 333 | 4 | 151 | 472 | hsa-miR-141 | 2432 | 21 | 0 |
| 334 | 4 | 218 | 527 | hsa-miR-143 | 4899 | 21 | 0 |
| 335 | 4 | 139 | 281 | hsa-miR-145 | 8885 | 21 | 0 |
| 336 | 4 | 212 | 333 | hsa-miR-149 | 641 | 21 | 0 |
| 337 | 4 | 34 | 396 | hsa-miR-151 | 560 | 21 | 0 |
| 338 | 4 | 172 | 329 | hsa-miR-15b | 692 | 21 | 0 |
| 339 | 4 | 173 | 360, 468 | hsa-miR-16 | 2504 | 21 | 0 |
| 340 | 4 | 38 | 467 | hsa-miR-17-3p | 534 | 21 | 0 |
| 341 | 4 | 75 | 467 | hsa-miR-17-5p | 1303 | 21 | 0 |
| 342 | 4 | 10 | 371, 461 | hsa-miR-181a | 1814 | 21 | 0 |
| 343 | 4 | 11 | 456, 540 | hsa-miR-181b | 669 | 21 | 0 |
| 344 | 4 | 179 | 356 | hsa-miR-183 | 676 | 21 | 0 |
| 345 | 4 | 234 | 450 | hsa-miR-185 | 649 | 21 | 0 |
| 346 | 4 | 77 | 440 | hsa-miR-191 | 1766 | 21 | 0 |
| 347 | 4 | 17 | 442 | hsa-miR-193a | 1448 | 21 | 0 |
| 348 | 4 | 16 | 488 | hsa-miR-193b | 671 | 21 | 0 |
| 349 | 4 | 92 | 410, 432 | hsa-miR-199a | 2940 | 21 | 0 |
| 350 | 4 | 162 | 410, 432, 473 | hsa-miR-199a* | 588 | 21 | 0 |
| 351 | 4 | 251 | 282, 495 | hsa-miR-19b | 1014 | 21 | 0 |
| 352 | 4 | 152 | 369 | hsa-miR-200a | 1007 | 21 | 0 |
| 353 | 4 | 157 | 411 | hsa-miR-200b | 1473 | 21 | 0 |
| 354 | 4 | 156 | 446 | hsa-miR-200c | 3113 | 21 | 0 |
| 355 | 4 | 203 | 504 | hsa-miR-205 | 6020 | 21 | 0 |
| 356 | 4 | 175 | 462 | hsa-miR-21 | 6697 | 21 | 0 |
| 357 | 4 | 115 | 314 | hsa-miR-210 | 757 | 21 | 0 |
| 358 | 4 | 29 | 439 | hsa-miR-214 | 1237 | 21 | 0 |
| 359 | 4 | 159 | 494 | hsa-miR-216 | 523 | 21 | 0 |
| 360 | 4 | 18 | 322 | hsa-miR-22 | 3021 | 21 | 0 |
| 361 | 4 | 50 | 364 | hsa-miR-221 | 2213 | 21 | 0 |
| 362 | 4 | 49 | 302 | hsa-miR-222 | 2428 | 21 | 0 |
| 363 | 4 | 67 | 336 | hsa-miR-23a | 2972 | 21 | 0 |
| 364 | 4 | 66 | 306 | hsa-miR-23b | 1202 | 21 | 1.23e−038 |
| 365 | 4 | 238 | 346, 416 | hsa-miR-24 | 8467 | 21 | 0 |
| 366 | 4 | 256 | 441, 487 | hsa-miR-26a | 4495 | 21 | 0 |
| 367 | 4 | 259 | 481 | hsa-miR-27a | 3358 | 21 | 0 |
| 368 | 4 | 260 | 280 | hsa-miR-27b | 1423 | 21 | 0 |
| 369 | 4 | 61 | 286 | hsa-miR-296 | 1045 | 21 | 0 |
| 370 | 4 | 243 | 299, 316 | hsa-miR-30c | 747 | 21 | 0 |
| 371 | 4 | 241 | 550 | hsa-miR-30d | 1269 | 21 | 0 |
| 372 | 4 | 245 | 431 | hsa-miR-30e-5p | 1336 | 21 | 0 |
| 373 | 4 | 132 | 429 | hsa-miR-31 | 1301 | 21 | 0 |
| 374 | 4 | 1 | 359 | hsa-miR-320 | 3910 | 21 | 0 |
| 375 | 4 | 91 | 368 | hsa-miR-324-3p | 681 | 21 | 0 |
| 376 | 4 | 98 | 368 | hsa-miR-324-5p | 571 | 21 | 0 |
| 377 | 4 | 113 | 538 | hsa-miR-328 | 645 | 21 | 0 |
| 378 | 4 | 126 | 380 | hsa-miR-331 | 667 | 21 | 0 |
| 379 | 4 | 194 | 337 | hsa-miR-338 | 679 | 21 | 0 |
| 380 | 4 | 208 | 477 | hsa-miR-342 | 790 | 21 | 0 |
| 381 | 4 | 229 | 292 | hsa-miR-345 | 577 | 21 | 0 |
| 382 | 4 | 237 | 480 | hsa-miR-34a | 1938 | 21 | 0 |
| 383 | 4 | 128 | 294 | hsa-miR-370 | 794 | 21 | 0 |
| 384 | 4 | 112 | 372 | hsa-miR-422b | 1038 | 21 | 0 |
| 385 | 4 | 51 | 497 | hsa-miR-423 | 1932 | 21 | 0 |
| 386 | 4 | 236 | 413 | hsa-miR-449 | 1525 | 21 | 0 |
| 387 | 4 | 63 | 557 | hsa-miR-491 | 2462 | 21 | 0 |
| 388 | 4 | 54 | 293 | hsa-miR-492 | 566 | 21 | 0 |
| 389 | 4 | 214 | 376 | hsa-miR-494 | 3804 | 21 | 0 |
| 390 | 4 | 83 | 312 | hsa-miR-497 | 776 | 21 | 0 |
| 391 | 4 | 43 | 418 | hsa-miR-504 | 689 | 21 | 0 |
| 392 | 4 | 258 | 428, 443 | hsa-miR-513 | 586 | 21 | 0 |
| 393 | 4 | 210 | 400 | hsa-miR-518c* | 526 | 21 | 0 |
| 394 | 4 | 206 | 513 | hsa-miR-520d* | 531 | 21 | 0 |
| 395 | 4 | 135 | 315 | hsa-miR-565 | 1605 | 19 | 0 |
| 396 | 4 | 60 | 479 | hsa-miR-638 | 8725 | 19 | 0 |
| 397 | 4 | 123 | 291 | hsa-miR-801 | 1348 | 9 | 2.63e−021 |
| 398 | 4 | 182 | 304, 317 | hsa-miR-92 | 2468 | 21 | 0 |
| 399 | 4 | 6 | 471 | hsa-miR-93 | 2127 | 21 | 0 |
| 400 | 4 | 14 | 318 | hsa-miR-99a | 1685 | 21 | 0 |
| 401 | 4 | 80 | 509 | hsa-miR-99b | 1029 | 21 | 0 |
| 402 | 4 | 131 | 430 | 6_11 | 526 | 21 | 0 |
| 403 | 5 | 127 | 361 | 11_1 | 772 | 12 | 2.41e−027 |
| 404 | 5 | 196 | 508 | 15_rel | 8319 | 10 | 2e−017 |
| 405 | 5 | 219 | 497 | 27_3 | 800 | 12 | 1.67e−024 |
| 406 | 5 | 129 | 548 | 43_39 | 638 | 12 | 2.41e−027 |
| 407 | 5 | 94 | 394 | 69_6_rel | 2043 | 10 | 1.08e−018 |
| 408 | 5 | 121 | 502 | 70_14 | 510 | 10 | 6.86e−023 |
| 409 | 5 | 177 | 366, 464 | 72_12 | 1070 | 10 | 2.15e−023 |
| 410 | 5 | 40 | 553 | 72_4 | 1636 | 10 | 7.13e−023 |
| 411 | 5 | 97 | 357 | 74_26_rel | 2411 | 10 | 1.41e−023 |
| 412 | 5 | 163 | 402 | 43_7 | 673 | 10 | 6.9e−025 |
| 413 | 5 | 223 | 320, 393, 559 | hsa-let-7a | 3219 | 12 | 1.3e−029 |
| 414 | 5 | 225 | 332 | hsa-let-7b | 8804 | 12 | 7.66e−030 |
| 415 | 5 | 224 | 427 | hsa-let-7c | 3791 | 12 | 1.53e−030 |
| 416 | 5 | 45 | 277 | hsa-let-7d | 2080 | 12 | 1.37e−030 |
| 417 | 5 | 220 | 475 | hsa-let-7e | 2262 | 12 | 3.98e−032 |
| 418 | 5 | 222 | 482, 558 | hsa-let-7f | 590 | 12 | 3.95e−030 |
| 419 | 5 | 226 | 395 | hsa-let-7g | 1189 | 12 | 1.17e−029 |
| 420 | 5 | 227 | 297 | hsa-let-7i | 1184 | 12 | 2.42e−030 |
| 421 | 5 | 48 | 483, 529 | hsa-miR-103 | 1295 | 12 | 1.35e−023 |
| 422 | 5 | 184 | 423 | hsa-miR-105 | 972 | 12 | 2.01e−031 |
| 423 | 5 | 2 | 399 | hsa-miR-106a | 1928 | 12 | 2.72e−031 |
| 424 | 5 | 149 | 392 | hsa-miR-106b | 1388 | 12 | 4.16e−033 |
| 425 | 5 | 47 | 555 | hsa-miR-107 | 2603 | 12 | 4.01e−029 |
| 426 | 5 | 199 | 387 | hsa-miR-125a | 1367 | 12 | 1.3e−028 |
| 427 | 5 | 198 | 313, 551 | hsa-miR-125b | 1466 | 12 | 5.12e−025 |
| 428 | 5 | 204 | 419 | hsa-miR-126 | 1518 | 12 | 1.62e−024 |
| 429 | 5 | 86 | 417 | hsa-miR-130a | 1547 | 12 | 1.11e−030 |
| 430 | 5 | 64 | 402 | hsa-miR-140 | 507 | 12 | 4.87e−027 |
| 431 | 5 | 151 | 472 | hsa-miR-141 | 5868 | 12 | 8.57e−031 |
| 432 | 5 | 218 | 527 | hsa-miR-143 | 2132 | 12 | 3.33e−022 |
| 433 | 5 | 139 | 281 | hsa-miR-145 | 2405 | 12 | 2.6e−024 |
| 434 | 5 | 212 | 333 | hsa-miR-149 | 783 | 12 | 2.05e−029 |
| 435 | 5 | 34 | 396 | hsa-miR-151 | 665 | 12 | 6.71e−030 |
| 436 | 5 | 172 | 329 | hsa-miR-15b | 516 | 12 | 2.82e−025 |
| 437 | 5 | 173 | 360, 468 | hsa-miR-16 | 2991 | 12 | 5.97e−030 |
| 438 | 5 | 75 | 467 | hsa-miR-17-5p | 1325 | 12 | 1.91e−031 |
| 439 | 5 | 10 | 371, 461 | hsa-miR-181a | 1188 | 12 | 1.51e−030 |
| 440 | 5 | 77 | 440 | hsa-miR-191 | 3392 | 12 | 2.99e−030 |
| 441 | 5 | 17 | 442 | hsa-miR-193a | 897 | 12 | 5.91e−027 |
| 442 | 5 | 92 | 410, 432 | hsa-miR-199a | 1233 | 12 | 1.8e−024 |
| 443 | 5 | 251 | 282, 495 | hsa-miR-19b | 612 | 12 | 5.47e−025 |
| 444 | 5 | 152 | 369 | hsa-miR-200a | 754 | 12 | 1.47e−022 |
| 445 | 5 | 157 | 411 | hsa-miR-200b | 1385 | 12 | 8.47e−024 |
| 446 | 5 | 156 | 446 | hsa-miR-200c | 6562 | 12 | 3.54e−032 |
| 447 | 5 | 203 | 504 | hsa-miR-205 | 12899 | 12 | 6.08e−030 |
| 448 | 5 | 175 | 462 | hsa-miR-21 | 3024 | 12 | 4.13e−029 |
| 449 | 5 | 115 | 314 | hsa-miR-210 | 1141 | 12 | 1.76e−032 |
| 450 | 5 | 29 | 439 | hsa-miR-214 | 967 | 12 | 9.09e−029 |
| 451 | 5 | 18 | 322 | hsa-miR-22 | 1812 | 12 | 3.21e−028 |
| 452 | 5 | 50 | 364 | hsa-miR-221 | 1180 | 12 | 4.94e−029 |
| 453 | 5 | 49 | 302 | hsa-miR-222 | 1507 | 12 | 2.27e−029 |
| 454 | 5 | 67 | 336 | hsa-miR-23a | 2962 | 12 | 1.4e−025 |
| 455 | 5 | 238 | 346, 416 | hsa-miR-24 | 6720 | 12 | 1.34e−030 |
| 456 | 5 | 256 | 441, 487 | hsa-miR-26a | 4328 | 12 | 1.89e−031 |
| 457 | 5 | 259 | 481 | hsa-miR-27a | 3145 | 12 | 6.88e−030 |
| 458 | 5 | 260 | 280 | hsa-miR-27b | 1467 | 12 | 3.21e−028 |
| 459 | 5 | 61 | 286 | hsa-miR-296 | 521 | 12 | 2.72e−024 |
| 460 | 5 | 243 | 299, 316 | hsa-miR-30c | 747 | 12 | 2.21e−024 |
| 461 | 5 | 241 | 550 | hsa-miR-30d | 1273 | 12 | 1.7e−029 |
| 462 | 5 | 245 | 431 | hsa-miR-30e-5p | 1458 | 12 | 6.18e−030 |
| 463 | 5 | 132 | 429 | hsa-miR-31 | 2299 | 12 | 8.6e−026 |
| 464 | 5 | 1 | 359 | hsa-miR-320 | 3276 | 12 | 7.25e−033 |
| 465 | 5 | 91 | 368 | hsa-miR-324-3p | 657 | 12 | 1.38e−033 |
| 466 | 5 | 113 | 538 | hsa-miR-328 | 669 | 12 | 3.33e−027 |
| 467 | 5 | 126 | 380 | hsa-miR-331 | 589 | 12 | 8.07e−029 |
| 468 | 5 | 208 | 477 | hsa-miR-342 | 678 | 12 | 7.71e−031 |
| 469 | 5 | 229 | 292 | hsa-miR-345 | 628 | 12 | 2.11e−027 |
| 470 | 5 | 237 | 480 | hsa-miR-34a | 2184 | 12 | 2.34e−030 |
| 471 | 5 | 128 | 294 | hsa-miR-370 | 562 | 12 | 1.18e−027 |
| 472 | 5 | 111 | 389 | hsa-miR-422a | 574 | 12 | 1.8e−031 |
| 473 | 5 | 112 | 372 | hsa-miR-422b | 1319 | 12 | 4.1e−030 |
| 474 | 5 | 51 | 497 | hsa-miR-423 | 2094 | 12 | 9.04e−032 |
| 475 | 5 | 158 | 378 | hsa-miR-429 | 514 | 12 | 2.7e−031 |
| 476 | 5 | 236 | 413 | hsa-miR-449 | 1472 | 12 | 5.92e−026 |
| 477 | 5 | 252 | 405 | hsa-miR-452 | 574 | 12 | 3.31e−026 |
| 478 | 5 | 63 | 557 | hsa-miR-491 | 1886 | 12 | 1.71e−028 |
| 479 | 5 | 54 | 293 | hsa-miR-492 | 518 | 12 | 1.68e−026 |
| 480 | 5 | 214 | 376 | hsa-miR-494 | 6177 | 12 | 6.55e−027 |
| 481 | 5 | 83 | 312 | hsa-miR-497 | 605 | 12 | 5.45e−027 |
| 482 | 5 | 43 | 418 | hsa-miR-504 | 566 | 12 | 2.84e−029 |
| 483 | 5 | 258 | 428, 443 | hsa-miR-513 | 714 | 12 | 1.44e−025 |
| 484 | 5 | 210 | 400 | hsa-miR-518c* | 507 | 12 | 7.21e−027 |
| 485 | 5 | 135 | 315 | hsa-miR-565 | 2606 | 10 | 2.37e−024 |
| 486 | 5 | 60 | 479 | hsa-miR-638 | 8078 | 10 | 6.19e−025 |
| 487 | 5 | 182 | 304, 317 | hsa-miR-92 | 1770 | 12 | 1.02e−029 |
| 488 | 5 | 6 | 471 | hsa-miR-93 | 2183 | 12 | 5.39e−029 |
| 489 | 5 | 14 | 318 | hsa-miR-99a | 573 | 12 | 1.08e−025 |
| 490 | 5 | 80 | 509 | hsa-miR-99b | 994 | 12 | 6.85e−030 |
| 491 | 5 | 131 | 430 | 6_11 | 669 | 12 | 5.03e−028 |
| 492 | 6 | 127 | 361 | 11_1 | 42409 | 5 | 3.25e−010 |
| 493 | 6 | 196 | 508 | 15_rel | 42818 | 5 | 1.12e−009 |
| 494 | 6 | 197 | 444 | 25_6 | 503 | 5 | 6.99e−014 |
| 495 | 6 | 219 | 497 | 27_3 | 3612 | 5 | 9.54e−012 |
| 496 | 6 | 129 | 548 | 43_39 | 512 | 5 | 4.09e−011 |
| 497 | 6 | 143 | 416 | 43_43 | 628 | 5 | 9.02e−009 |
| 498 | 6 | 114 | 530 | 43_6 | 1394 | 5 | 2.61e−010 |
| 499 | 6 | 94 | 394 | 69_6_rel | 12806 | 5 | 1.28e−009 |
| 500 | 6 | 121 | 502 | 70_14 | 780 | 5 | 1.35e−008 |
| 501 | 6 | 177 | 366, 464 | 72_12 | 28875 | 5 | 3.99e−011 |
| 502 | 6 | 40 | 553 | 72_4 | 22990 | 5 | 2.01e−010 |
| 503 | 6 | 97 | 357 | 74_26_rel | 2884 | 5 | 8.95e−010 |
| 504 | 6 | 87 | 301 | gam11 | 19450 | 5 | 7.72e−009 |
| 505 | 6 | 163 | 402 | 43_7 | 2496 | 5 | 8.15e−012 |
| 506 | 6 | 223 | 320, 393, 559 | hsa-let-7a | 48931 | 5 | 2.53e−012 |
| 507 | 6 | 225 | 332 | hsa-let-7b | 64899 | 5 | 1.03e−010 |
| 508 | 6 | 224 | 427 | hsa-let-7c | 56829 | 5 | 4.87e−011 |
| 509 | 6 | 45 | 277 | hsa-let-7d | 20527 | 5 | 8.66e−011 |
| 510 | 6 | 220 | 475 | hsa-let-7e | 18351 | 5 | 1.42e−010 |
| 511 | 6 | 222 | 482, 558 | hsa-let-7f | 1843 | 5 | 5.56e−012 |
| 512 | 6 | 226 | 395 | hsa-let-7g | 2958 | 5 | 1.18e−012 |
| 513 | 6 | 227 | 297 | hsa-let-7i | 5532 | 5 | 8.77e−011 |
| 514 | 6 | 13 | 397 | hsa-miR-100 | 2410 | 5 | 2.35e−009 |
| 515 | 6 | 48 | 483, 529 | hsa-miR-103 | 46584 | 5 | 1.64e−010 |
| 516 | 6 | 184 | 423 | hsa-miR-105 | 7918 | 5 | 5.13e−010 |
| 517 | 6 | 2 | 399 | hsa-miR-106a | 18541 | 5 | 3.72e−010 |
| 518 | 6 | 149 | 392 | hsa-miR-106b | 20071 | 5 | 1.12e−010 |
| 519 | 6 | 47 | 555 | hsa-miR-107 | 39685 | 5 | 2.15e−010 |
| 520 | 6 | 166 | 476 | hsa-miR-10a | 1319 | 5 | 4.66e−009 |
| 521 | 6 | 165 | 340 | hsa-miR-10b | 997 | 5 | 5.62e−009 |
| 522 | 6 | 199 | 387 | hsa-miR-125a | 11523 | 5 | 6.13e−011 |
| 523 | 6 | 198 | 313, 551 | hsa-miR-125b | 6898 | 5 | 1.61e−009 |
| 524 | 6 | 204 | 419 | hsa-miR-126 | 20486 | 5 | 2.02e−011 |
| 525 | 6 | 188 | 491 | hsa-miR-128a | 1110 | 5 | 5.81e−011 |
| 526 | 6 | 187 | 383 | hsa-miR-128b | 982 | 5 | 2.61e−010 |
| 527 | 6 | 86 | 417 | hsa-miR-130a | 4549 | 5 | 8.48e−011 |
| 528 | 6 | 85 | 328 | hsa-miR-130b | 1676 | 5 | 8.83e−011 |
| 529 | 6 | 37 | 391 | hsa-miR-136 | 834 | 5 | 5.51e−009 |
| 530 | 6 | 52 | 370, 548 | hsa-miR-138 | 534 | 5 | 1.1e−010 |
| 531 | 6 | 64 | 402 | hsa-miR-140 | 554 | 5 | 2.23e−009 |
| 532 | 6 | 151 | 472 | hsa-miR-141 | 51355 | 5 | 4.9e−012 |
| 533 | 6 | 139 | 281 | hsa-miR-145 | 24078 | 5 | 2.28e−009 |
| 534 | 6 | 190 | 458 | hsa-miR-148a | 1182 | 5 | 1.35e−009 |
| 535 | 6 | 191 | 289 | hsa-miR-148b | 3391 | 5 | 1.12e−010 |
| 536 | 6 | 212 | 333 | hsa-miR-149 | 2973 | 5 | 2.91e−010 |
| 537 | 6 | 209 | 507 | hsa-miR-150 | 1217 | 5 | 2.46e−009 |
| 538 | 6 | 34 | 396 | hsa-miR-151 | 4386 | 5 | 1.14e−010 |
| 539 | 6 | 192 | 470 | hsa-miR-152 | 1028 | 5 | 5.96e−010 |
| 540 | 6 | 171 | 331 | hsa-miR-15a | 3069 | 5 | 4.58e−010 |
| 541 | 6 | 172 | 329 | hsa-miR-15b | 11137 | 5 | 1.33e−010 |
| 542 | 6 | 173 | 360, 468 | hsa-miR-16 | 54954 | 5 | 1.06e−010 |
| 543 | 6 | 38 | 467 | hsa-miR-17-3p | 4029 | 5 | 2.71e−010 |
| 544 | 6 | 75 | 467 | hsa-miR-17-5p | 16629 | 5 | 3.27e−010 |
| 545 | 6 | 10 | 371, 461 | hsa-miR-181a | 6279 | 5 | 8.04e−012 |
| 546 | 6 | 11 | 456, 540 | hsa-miR-181b | 2587 | 5 | 4.69e−011 |
| 547 | 6 | 9 | 451 | hsa-miR-181c | 2162 | 5 | 2.92e−010 |
| 548 | 6 | 272 | 323 | hsa-miR-182 | 5527 | 5 | 1.38e−010 |
| 549 | 6 | 179 | 356 | hsa-miR-183 | 2146 | 5 | 2.87e−010 |
| 550 | 6 | 234 | 450 | hsa-miR-185 | 3595 | 5 | 2.1e−010 |
| 551 | 6 | 205 | 367 | hsa-miR-187 | 1393 | 5 | 5.85e−009 |
| 552 | 6 | 88 | 310 | hsa-miR-188 | 501 | 5 | 2.41e−010 |
| 553 | 6 | 155 | 426 | hsa-miR-18a | 2908 | 5 | 5.06e−010 |
| 554 | 6 | 77 | 440 | hsa-miR-191 | 37448 | 5 | 6.43e−011 |
| 555 | 6 | 17 | 442 | hsa-miR-193a | 10366 | 5 | 2.14e−009 |
| 556 | 6 | 16 | 488 | hsa-miR-193b | 5948 | 5 | 4.68e−010 |
| 557 | 6 | 246 | 290, 549 | hsa-miR-194 | 962 | 5 | 1.47e−010 |
| 558 | 6 | 170 | 327 | hsa-miR-195 | 2890 | 5 | 1.54e−010 |
| 559 | 6 | 92 | 410, 432 | hsa-miR-199a | 12143 | 5 | 1.02e−009 |
| 560 | 6 | 162 | 410, 432, 473 | hsa-miR-199a* | 2500 | 5 | 9.68e−010 |
| 561 | 6 | 251 | 282, 495 | hsa-miR-19b | 18534 | 5 | 8.96e−011 |
| 562 | 6 | 152 | 369 | hsa-miR-200a | 3469 | 5 | 5e−007 |
| 563 | 6 | 157 | 411 | hsa-miR-200b | 47312 | 5 | 1.05e−010 |
| 564 | 6 | 156 | 446 | hsa-miR-200c | 58727 | 5 | 1.62e−011 |
| 565 | 6 | 140 | 420 | hsa-miR-203 | 4439 | 5 | 1.35e−008 |
| 566 | 6 | 203 | 504 | hsa-miR-205 | 67013 | 5 | 9.73e−011 |
| 567 | 6 | 150 | 351 | hsa-miR-20a | 2559 | 5 | 6.3e−010 |
| 568 | 6 | 175 | 462 | hsa-miR-21 | 26992 | 5 | 1.13e−010 |
| 569 | 6 | 115 | 314 | hsa-miR-210 | 10976 | 5 | 1.34e−009 |
| 570 | 6 | 29 | 439 | hsa-miR-214 | 1389 | 5 | 5.6e−012 |
| 571 | 6 | 18 | 322 | hsa-miR-22 | 30642 | 5 | 1.59e−011 |
| 572 | 6 | 50 | 364 | hsa-miR-221 | 12239 | 5 | 3.56e−011 |
| 573 | 6 | 49 | 302 | hsa-miR-222 | 9426 | 5 | 1.23e−010 |
| 574 | 6 | 67 | 336 | hsa-miR-23a | 44362 | 5 | 1.45e−011 |
| 575 | 6 | 238 | 346, 416 | hsa-miR-24 | 63449 | 5 | 5.65e−011 |
| 576 | 6 | 90 | 388 | hsa-miR-25 | 4729 | 5 | 2.46e−010 |
| 577 | 6 | 256 | 441, 487 | hsa-miR-26a | 58909 | 5 | 1.93e−011 |
| 578 | 6 | 257 | 409 | hsa-miR-26b | 730 | 5 | 1.66e−010 |
| 579 | 6 | 259 | 481 | hsa-miR-27a | 50562 | 5 | 8.21e−011 |
| 580 | 6 | 260 | 280 | hsa-miR-27b | 17331 | 5 | 3.02e−010 |
| 581 | 6 | 19 | 457 | hsa-miR-28 | 891 | 5 | 1.45e−010 |
| 582 | 6 | 169 | 528 | hsa-miR-29c | 2012 | 5 | 1.77e−010 |
| 583 | 6 | 84 | 489 | hsa-miR-301 | 544 | 5 | 1.13e−009 |
| 584 | 6 | 242 | 311 | hsa-miR-30b | 5975 | 5 | 1.7e−011 |
| 585 | 6 | 243 | 299, 316 | hsa-miR-30c | 26163 | 5 | 4.66e−012 |
| 586 | 6 | 241 | 550 | hsa-miR-30d | 18865 | 5 | 6.51e−011 |
| 587 | 6 | 245 | 431 | hsa-miR-30e-5p | 23523 | 5 | 2.51e−011 |
| 588 | 6 | 132 | 429 | hsa-miR-31 | 23553 | 5 | 2.17e−008 |
| 589 | 6 | 1 | 359 | hsa-miR-320 | 12299 | 5 | 1.64e−011 |
| 590 | 6 | 91 | 368 | hsa-miR-324-3p | 2138 | 5 | 5.71e−012 |
| 591 | 6 | 98 | 368 | hsa-miR-324-5p | 1104 | 5 | 1.33e−013 |
| 592 | 6 | 126 | 380 | hsa-miR-331 | 3338 | 5 | 7.96e−011 |
| 593 | 6 | 200 | 448 | hsa-miR-339 | 2301 | 5 | 4.2e−010 |
| 594 | 6 | 208 | 477 | hsa-miR-342 | 2790 | 5 | 3.81e−010 |
| 595 | 6 | 229 | 292 | hsa-miR-345 | 1859 | 5 | 2.97e−010 |
| 596 | 6 | 237 | 480 | hsa-miR-34a | 29294 | 5 | 1.86e−011 |
| 597 | 6 | 57 | 498 | hsa-miR-34c | 1101 | 5 | 2.28e−010 |
| 598 | 6 | 255 | 444 | hsa-miR-361 | 2934 | 5 | 4.06e−011 |
| 599 | 6 | 160 | 379, 382 | hsa-miR-365 | 993 | 5 | 7.23e−012 |
| 600 | 6 | 31 | 560 | hsa-miR-368 | 699 | 5 | 3.78e−010 |
| 601 | 6 | 128 | 294 | hsa-miR-370 | 736 | 5 | 7.27e−010 |
| 602 | 6 | 105 | 372 | hsa-miR-378 | 547 | 5 | 1.97e−010 |
| 603 | 6 | 111 | 389 | hsa-miR-422a | 1743 | 5 | 5.51e−011 |
| 604 | 6 | 112 | 372 | hsa-miR-422b | 6127 | 5 | 2.23e−010 |
| 605 | 6 | 51 | 497 | hsa-miR-423 | 6401 | 5 | 8.3e−012 |
| 606 | 6 | 69 | 363 | hsa-miR-425-3p | 1399 | 5 | 1e−010 |
| 607 | 6 | 158 | 378 | hsa-miR-429 | 4315 | 5 | 3.8e−010 |
| 608 | 6 | 236 | 413 | hsa-miR-449 | 697 | 5 | 1.11e−008 |
| 609 | 6 | 4 | 430 | hsa-miR-451 | 2823 | 5 | 4.53e−010 |
| 610 | 6 | 252 | 405 | hsa-miR-452 | 697 | 5 | 6.02e−011 |
| 611 | 6 | 189 | 500 | hsa-miR-484 | 1781 | 5 | 4.2e−010 |
| 612 | 6 | 63 | 557 | hsa-miR-491 | 1975 | 5 | 6.97e−010 |
| 613 | 6 | 54 | 293 | hsa-miR-492 | 743 | 5 | 7.47e−009 |
| 614 | 6 | 214 | 376 | hsa-miR-494 | 8145 | 5 | 1.63e−009 |
| 615 | 6 | 83 | 312 | hsa-miR-497 | 5233 | 5 | 1.36e−010 |
| 616 | 6 | 43 | 418 | hsa-miR-504 | 1039 | 5 | 2.71e−009 |
| 617 | 6 | 137 | 542 | hsa-miR-505 | 881 | 5 | 2.6e−010 |
| 618 | 6 | 258 | 428, 443 | hsa-miR-513 | 750 | 5 | 2.66e−009 |
| 619 | 6 | 8 | 276 | hsa-miR-552 | 635 | 5 | 3.23e−009 |
| 620 | 6 | 135 | 315 | hsa-miR-565 | 10240 | 5 | 8.77e−010 |
| 621 | 6 | 60 | 479 | hsa-miR-638 | 13158 | 5 | 2.07e−011 |
| 622 | 6 | 164 | 381 | hsa-miR-660 | 743 | 5 | 1.28e−010 |
| 623 | 6 | 195 | 421 | hsa-miR-662 | 525 | 5 | 3.36e−009 |
| 624 | 6 | 182 | 304, 317 | hsa-miR-92 | 18804 | 5 | 1.02e−010 |
| 625 | 6 | 6 | 471 | hsa-miR-93 | 26470 | 5 | 2.84e−010 |
| 626 | 6 | 221 | 307 | hsa-miR-98 | 551 | 5 | 1.17e−011 |
| 627 | 6 | 14 | 318 | hsa-miR-99a | 3430 | 5 | 5.81e−009 |
| 628 | 6 | 80 | 509 | hsa-miR-99b | 3479 | 5 | 1.31e−010 |
| 629 | 6 | 131 | 430 | 6_11 | 3134 | 5 | 1.01e−010 |
| 630 | 6 | 215 | 528 | 43_5 | 1881 | 5 | 8.61e−010 |
| 631 | 7 | 127 | 361 | 11_1 | 562 | 6 | 2.39e−014 |
| 632 | 7 | 196 | 508 | 15_rel | 35532 | 3 | 6.73e−006 |
| 633 | 7 | 219 | 497 | 27_3 | 702 | 6 | 2.84e−014 |
| 634 | 7 | 129 | 548 | 43_39 | 1364 | 6 | 2.19e−015 |
| 635 | 7 | 94 | 394 | 69_6_rel | 18567 | 3 | 1.82e−006 |
| 636 | 7 | 177 | 366, 464 | 72_12 | 1323 | 3 | 1.96e−006 |
| 637 | 7 | 40 | 553 | 72_4 | 1153 | 3 | 3.21e−006 |
| 638 | 7 | 97 | 357 | 74_26_rel | 1229 | 3 | 1.64e−006 |
| 639 | 7 | 87 | 301 | gam11 | 12707 | 3 | 3.79e−007 |
| 640 | 7 | 163 | 402 | 43_7 | 847 | 3 | 8.11e−008 |
| 641 | 7 | 223 | 320, 393, 559 | hsa-let-7a | 8569 | 6 | 3.7e−015 |
| 642 | 7 | 225 | 332 | hsa-let-7b | 15795 | 6 | 3.91e−016 |
| 643 | 7 | 224 | 427 | hsa-let-7c | 12656 | 6 | 2.48e−017 |
| 644 | 7 | 45 | 277 | hsa-let-7d | 2942 | 6 | 2.21e−015 |
| 645 | 7 | 220 | 475 | hsa-let-7e | 2441 | 6 | 2.68e−018 |
| 646 | 7 | 222 | 482, 558 | hsa-let-7f | 1025 | 6 | 1.75e−014 |
| 647 | 7 | 226 | 395 | hsa-let-7g | 1543 | 6 | 5.85e−016 |
| 648 | 7 | 227 | 297 | hsa-let-7i | 1897 | 6 | 1.41e−016 |
| 649 | 7 | 13 | 397 | hsa-miR-100 | 1760 | 6 | 2.35e−015 |
| 650 | 7 | 48 | 483, 529 | hsa-miR-103 | 1399 | 6 | 6.46e−015 |
| 651 | 7 | 184 | 423 | hsa-miR-105 | 962 | 6 | 9.86e−017 |
| 652 | 7 | 2 | 399 | hsa-miR-106a | 1104 | 6 | 1.67e−016 |
| 653 | 7 | 149 | 392 | hsa-miR-106b | 735 | 6 | 1.92e−015 |
| 654 | 7 | 47 | 555 | hsa-miR-107 | 1293 | 6 | 1.91e−015 |
| 655 | 7 | 199 | 387 | hsa-miR-125a | 3007 | 6 | 5.66e−015 |
| 656 | 7 | 198 | 313, 551 | hsa-miR-125b | 12529 | 6 | 3.69e−015 |
| 657 | 7 | 204 | 419 | hsa-miR-126 | 3944 | 6 | 1.16e−014 |
| 658 | 7 | 89 | 419 | hsa-miR-126* | 558 | 6 | 1.06e−016 |
| 659 | 7 | 86 | 417 | hsa-miR-130a | 2244 | 6 | 6.92e−017 |
| 660 | 7 | 151 | 472 | hsa-miR-141 | 1202 | 6 | 2.01e−015 |
| 661 | 7 | 218 | 527 | hsa-miR-143 | 7200 | 6 | 2.44e−015 |
| 662 | 7 | 139 | 281 | hsa-miR-145 | 5915 | 6 | 1.19e−014 |
| 663 | 7 | 212 | 333 | hsa-miR-149 | 555 | 6 | 1.32e−015 |
| 664 | 7 | 209 | 507 | hsa-miR-150 | 665 | 6 | 2.77e−016 |
| 665 | 7 | 172 | 329 | hsa-miR-15b | 1338 | 6 | 5.89e−016 |
| 666 | 7 | 173 | 360, 468 | hsa-miR-16 | 3948 | 6 | 4.67e−016 |
| 667 | 7 | 75 | 467 | hsa-miR-17-5p | 990 | 6 | 1.95e−016 |
| 668 | 7 | 10 | 371, 461 | hsa-miR-181a | 1356 | 6 | 9.14e−016 |
| 669 | 7 | 234 | 450 | hsa-miR-185 | 592 | 6 | 2.15e−017 |
| 670 | 7 | 77 | 440 | hsa-miR-191 | 1493 | 6 | 3.81e−016 |
| 671 | 7 | 130 | 440 | hsa-miR-191* | 543 | 3 | 4.04e−007 |
| 672 | 7 | 17 | 442 | hsa-miR-193a | 3684 | 6 | 2.11e−016 |
| 673 | 7 | 16 | 488 | hsa-miR-193b | 1223 | 6 | 1.59e−015 |
| 674 | 7 | 170 | 327 | hsa-miR-195 | 2166 | 6 | 9.49e−015 |
| 675 | 7 | 92 | 410, 432 | hsa-miR-199a | 4337 | 6 | 3.99e−016 |
| 676 | 7 | 162 | 410, 432, 473 | hsa-miR-199a* | 2227 | 6 | 1.93e−014 |
| 677 | 7 | 93 | 473 | hsa-miR-199b | 1182 | 6 | 6.64e−015 |
| 678 | 7 | 251 | 282, 495 | hsa-miR-19b | 1339 | 6 | 2.58e−015 |
| 679 | 7 | 152 | 369 | hsa-miR-200a | 533 | 6 | 6.65e−017 |
| 680 | 7 | 157 | 411 | hsa-miR-200b | 934 | 6 | 1.16e−016 |
| 681 | 7 | 156 | 446 | hsa-miR-200c | 2111 | 6 | 3.49e−016 |
| 682 | 7 | 271 | 334 | hsa-miR-202* | 615 | 6 | 2.89e−015 |
| 683 | 7 | 203 | 504 | hsa-miR-205 | 6456 | 6 | 3.77e−014 |
| 684 | 7 | 175 | 462 | hsa-miR-21 | 2865 | 6 | 1.95e−014 |
| 685 | 7 | 29 | 439 | hsa-miR-214 | 1850 | 6 | 2.89e−017 |
| 686 | 7 | 18 | 322 | hsa-miR-22 | 1957 | 6 | 2.38e−015 |
| 687 | 7 | 50 | 364 | hsa-miR-221 | 1108 | 6 | 8.54e−016 |
| 688 | 7 | 49 | 302 | hsa-miR-222 | 1487 | 6 | 5.41e−016 |
| 689 | 7 | 67 | 336 | hsa-miR-23a | 4369 | 6 | 1.86e−015 |
| 690 | 7 | 66 | 306 | hsa-miR-23b | 1311 | 6 | 2.89e−012 |
| 691 | 7 | 238 | 346, 416 | hsa-miR-24 | 5912 | 6 | 1.21e−015 |
| 692 | 7 | 90 | 388 | hsa-miR-25 | 681 | 6 | 2.47e−018 |
| 693 | 7 | 256 | 441, 487 | hsa-miR-26a | 8543 | 6 | 6.19e−017 |
| 694 | 7 | 257 | 409 | hsa-miR-26b | 853 | 6 | 8.63e−016 |
| 695 | 7 | 259 | 481 | hsa-miR-27a | 5330 | 6 | 3.45e−017 |
| 696 | 7 | 260 | 280 | hsa-miR-27b | 1334 | 6 | 9.17e−016 |
| 697 | 7 | 61 | 286 | hsa-miR-296 | 502 | 6 | 5.1e−013 |
| 698 | 7 | 167 | 556 | hsa-miR-29a | 878 | 6 | 1.72e−012 |
| 699 | 7 | 117 | 287 | hsa-miR-30a-3p | 567 | 6 | 8.53e−017 |
| 700 | 7 | 244 | 287 | hsa-miR-30a-5p | 754 | 6 | 1.16e−012 |
| 701 | 7 | 242 | 311 | hsa-miR-30b | 1023 | 6 | 1.56e−014 |
| 702 | 7 | 243 | 299, 316 | hsa-miR-30c | 1615 | 6 | 4.94e−015 |
| 703 | 7 | 241 | 550 | hsa-miR-30d | 1126 | 6 | 4.68e−015 |
| 704 | 7 | 245 | 431 | hsa-miR-30e-5p | 1181 | 6 | 3.64e−016 |
| 705 | 7 | 132 | 429 | hsa-miR-31 | 523 | 6 | 1.07e−014 |
| 706 | 7 | 1 | 359 | hsa-miR-320 | 1732 | 6 | 7.38e−018 |
| 707 | 7 | 98 | 368 | hsa-miR-324-5p | 543 | 6 | 4.01e−015 |
| 708 | 7 | 113 | 538 | hsa-miR-328 | 997 | 6 | 6.02e−015 |
| 709 | 7 | 141 | 437 | hsa-miR-33 | 513 | 6 | 4.8e−014 |
| 710 | 7 | 208 | 477 | hsa-miR-342 | 863 | 6 | 4.08e−015 |
| 711 | 7 | 237 | 480 | hsa-miR-34a | 1571 | 6 | 1.52e−017 |
| 712 | 7 | 160 | 379, 382 | hsa-miR-365 | 817 | 6 | 3.72e−017 |
| 713 | 7 | 112 | 372 | hsa-miR-422b | 745 | 6 | 7.49e−016 |
| 714 | 7 | 51 | 497 | hsa-miR-423 | 1475 | 6 | 1.46e−015 |
| 715 | 7 | 236 | 413 | hsa-miR-449 | 1368 | 6 | 1.62e−014 |
| 716 | 7 | 63 | 557 | hsa-miR-491 | 1724 | 6 | 1.17e−014 |
| 717 | 7 | 214 | 376 | hsa-miR-494 | 9300 | 6 | 6.35e−016 |
| 718 | 7 | 83 | 312 | hsa-miR-497 | 2408 | 6 | 7.62e−015 |
| 719 | 7 | 258 | 428, 443 | hsa-miR-513 | 582 | 6 | 2.95e−014 |
| 720 | 7 | 135 | 315 | hsa-miR-565 | 1007 | 3 | 6.96e−007 |
| 721 | 7 | 60 | 479 | hsa-miR-638 | 15388 | 3 | 6.28e−006 |
| 722 | 7 | 182 | 304, 317 | hsa-miR-92 | 1399 | 6 | 1.61e−015 |
| 723 | 7 | 6 | 471 | hsa-miR-93 | 714 | 6 | 1.58e−015 |
| 724 | 7 | 14 | 318 | hsa-miR-99a | 5129 | 6 | 7.64e−016 |
| 725 | 7 | 80 | 509 | hsa-miR-99b | 686 | 6 | 2.27e−017 |
| 726 | 7 | 131 | 430 | 6_11 | 1289 | 6 | 2.3e−014 |
| 727 | 8 | 127 | 361 | 11_1 | 1116 | 15 | 0 |
| 728 | 8 | 196 | 508 | 15_rel | 58123 | 13 | 0 |
| 729 | 8 | 197 | 444 | 25_6 | 620 | 15 | 0 |
| 730 | 8 | 219 | 497 | 27_3 | 2013 | 15 | 0 |
| 731 | 8 | 42 | 449 | 43_3 | 591 | 15 | 3.55e−038 |
| 732 | 8 | 101 | 475 | 43_30 | 588 | 15 | 0 |
| 733 | 8 | 99 | 348, 384, 434 | 43_35 | 597 | 15 | 0 |
| 734 | 8 | 129 | 548 | 43_39 | 1988 | 15 | 0 |
| 735 | 8 | 114 | 530 | 43_6 | 643 | 15 | 0 |
| 736 | 8 | 183 | 373 | 66_1 | 839 | 15 | 0 |
| 737 | 8 | 94 | 394 | 69_6_rel | 18812 | 13 | 1.04e−033 |
| 738 | 8 | 121 | 502 | 70_14 | 958 | 13 | 1.76e−034 |
| 739 | 8 | 177 | 366, 464 | 72_12 | 2179 | 13 | 4.05e−033 |
| 740 | 8 | 40 | 553 | 72_4 | 2625 | 13 | 5.63e−034 |
| 741 | 8 | 97 | 357 | 74_26_rel | 4418 | 13 | 6.03e−036 |
| 742 | 8 | 87 | 301 | gam11 | 13043 | 13 | 3.31e−029 |
| 743 | 8 | 163 | 402 | 43_7 | 779 | 13 | 3.32e−033 |
| 744 | 8 | 223 | 320, 393, 559 | hsa-let-7a | 12387 | 15 | 0 |
| 745 | 8 | 225 | 332 | hsa-let-7b | 22223 | 15 | 0 |
| 746 | 8 | 224 | 427 | hsa-let-7c | 17133 | 15 | 0 |
| 747 | 8 | 45 | 277 | hsa-let-7d | 3570 | 15 | 0 |
| 748 | 8 | 220 | 475 | hsa-let-7e | 4100 | 15 | 0 |
| 749 | 8 | 222 | 482, 558 | hsa-let-7f | 1213 | 15 | 0 |
| 750 | 8 | 226 | 395 | hsa-let-7g | 2351 | 15 | 0 |
| 751 | 8 | 227 | 297 | hsa-let-7i | 2450 | 15 | 0 |
| 752 | 8 | 231 | 321, 424 | hsa-miR-1 | 567 | 15 | 0 |
| 753 | 8 | 13 | 397 | hsa-miR-100 | 1407 | 15 | 0 |
| 754 | 8 | 48 | 483, 529 | hsa-miR-103 | 2597 | 15 | 0 |
| 755 | 8 | 184 | 423 | hsa-miR-105 | 1617 | 15 | 0 |
| 756 | 8 | 2 | 399 | hsa-miR-106a | 2084 | 15 | 0 |
| 757 | 8 | 149 | 392 | hsa-miR-106b | 1822 | 15 | 0 |
| 758 | 8 | 47 | 555 | hsa-miR-107 | 2637 | 15 | 0 |
| 759 | 8 | 199 | 387 | hsa-miR-125a | 2841 | 15 | 0 |
| 760 | 8 | 198 | 313, 551 | hsa-miR-125b | 6941 | 15 | 0 |
| 761 | 8 | 204 | 419 | hsa-miR-126 | 2655 | 15 | 0 |
| 762 | 8 | 89 | 419 | hsa-miR-126* | 886 | 15 | 0 |
| 763 | 8 | 86 | 417 | hsa-miR-130a | 3575 | 15 | 0 |
| 764 | 8 | 85 | 328 | hsa-miR-130b | 600 | 15 | 7.55e−038 |
| 765 | 8 | 181 | 324, 554 | hsa-miR-135a | 597 | 15 | 6.01e−038 |
| 766 | 8 | 180 | 486 | hsa-miR-135b | 579 | 15 | 4.88e−038 |
| 767 | 8 | 37 | 391 | hsa-miR-136 | 573 | 15 | 5.59e−037 |
| 768 | 8 | 52 | 370, 548 | hsa-miR-138 | 719 | 15 | 0 |
| 769 | 8 | 64 | 402 | hsa-miR-140 | 1142 | 15 | 0 |
| 770 | 8 | 151 | 472 | hsa-miR-141 | 4309 | 15 | 0 |
| 771 | 8 | 218 | 527 | hsa-miR-143 | 4520 | 15 | 0 |
| 772 | 8 | 139 | 281 | hsa-miR-145 | 3556 | 15 | 0 |
| 773 | 8 | 217 | 466 | hsa-miR-146a | 504 | 15 | 0 |
| 774 | 8 | 146 | 325 | hsa-miR-147 | 675 | 15 | 0 |
| 775 | 8 | 190 | 458 | hsa-miR-148a | 693 | 15 | 0 |
| 776 | 8 | 191 | 289 | hsa-miR-148b | 784 | 15 | 0 |
| 777 | 8 | 212 | 333 | hsa-miR-149 | 1337 | 15 | 0 |
| 778 | 8 | 209 | 507 | hsa-miR-150 | 819 | 15 | 0 |
| 779 | 8 | 34 | 396 | hsa-miR-151 | 780 | 15 | 0 |
| 780 | 8 | 192 | 470 | hsa-miR-152 | 740 | 15 | 0 |
| 781 | 8 | 263 | 296, 404 | hsa-miR-153 | 540 | 15 | 0 |
| 782 | 8 | 253 | 499 | hsa-miR-155 | 544 | 15 | 0 |
| 783 | 8 | 171 | 331 | hsa-miR-15a | 807 | 15 | 0 |
| 784 | 8 | 172 | 329 | hsa-miR-15b | 1777 | 15 | 0 |
| 785 | 8 | 173 | 360, 468 | hsa-miR-16 | 4671 | 15 | 0 |
| 786 | 8 | 38 | 467 | hsa-miR-17-3p | 811 | 15 | 0 |
| 787 | 8 | 75 | 467 | hsa-miR-17-5p | 1743 | 15 | 0 |
| 788 | 8 | 10 | 371, 461 | hsa-miR-181a | 2857 | 15 | 0 |
| 789 | 8 | 11 | 456, 540 | hsa-miR-181b | 814 | 15 | 2.13e−038 |
| 790 | 8 | 9 | 451 | hsa-miR-181c | 554 | 15 | 0 |
| 791 | 8 | 272 | 323 | hsa-miR-182 | 654 | 15 | 1.87e−038 |
| 792 | 8 | 179 | 356 | hsa-miR-183 | 1906 | 15 | 1.62e−037 |
| 793 | 8 | 234 | 450 | hsa-miR-185 | 937 | 15 | 0 |
| 794 | 8 | 88 | 310 | hsa-miR-188 | 575 | 15 | 4.54e−037 |
| 795 | 8 | 144 | 346 | hsa-miR-189 | 750 | 15 | 0 |
| 796 | 8 | 155 | 426 | hsa-miR-18a | 506 | 15 | 0 |
| 797 | 8 | 77 | 440 | hsa-miR-191 | 2941 | 15 | 0 |
| 798 | 8 | 130 | 440 | hsa-miR-191* | 565 | 13 | 1.16e−034 |
| 799 | 8 | 17 | 442 | hsa-miR-193a | 5469 | 15 | 0 |
| 800 | 8 | 16 | 488 | hsa-miR-193b | 1578 | 15 | 1.93e−036 |
| 801 | 8 | 170 | 327 | hsa-miR-195 | 1553 | 15 | 0 |
| 802 | 8 | 136 | 308 | hsa-miR-198 | 625 | 15 | 0 |
| 803 | 8 | 92 | 410, 432 | hsa-miR-199a | 5007 | 15 | 0 |
| 804 | 8 | 162 | 410, 432, 473 | hsa-miR-199a* | 1519 | 15 | 3.49e−036 |
| 805 | 8 | 93 | 473 | hsa-miR-199b | 746 | 15 | 0 |
| 806 | 8 | 251 | 282, 495 | hsa-miR-19b | 1810 | 15 | 0 |
| 807 | 8 | 152 | 369 | hsa-miR-200a | 1362 | 15 | 0 |
| 808 | 8 | 157 | 411 | hsa-miR-200b | 2341 | 15 | 0 |
| 809 | 8 | 156 | 446 | hsa-miR-200c | 5325 | 15 | 0 |
| 810 | 8 | 271 | 334 | hsa-miR-202* | 844 | 15 | 6.23e−038 |
| 811 | 8 | 203 | 504 | hsa-miR-205 | 4678 | 15 | 3.13e−036 |
| 812 | 8 | 232 | 422 | hsa-miR-206 | 693 | 15 | 0 |
| 813 | 8 | 150 | 351 | hsa-miR-20a | 615 | 15 | 0 |
| 814 | 8 | 175 | 462 | hsa-miR-21 | 6975 | 15 | 0 |
| 815 | 8 | 115 | 314 | hsa-miR-210 | 673 | 15 | 0 |
| 816 | 8 | 29 | 439 | hsa-miR-214 | 2737 | 15 | 0 |
| 817 | 8 | 159 | 494 | hsa-miR-216 | 786 | 15 | 0 |
| 818 | 8 | 268 | 401, 425 | hsa-miR-218 | 610 | 15 | 0 |
| 819 | 8 | 18 | 322 | hsa-miR-22 | 2600 | 15 | 0 |
| 820 | 8 | 50 | 364 | hsa-miR-221 | 1610 | 15 | 0 |
| 821 | 8 | 49 | 302 | hsa-miR-222 | 2383 | 15 | 0 |
| 822 | 8 | 248 | 415 | hsa-miR-223 | 630 | 15 | 0 |
| 823 | 8 | 67 | 336 | hsa-miR-23a | 4138 | 15 | 0 |
| 824 | 8 | 238 | 346, 416 | hsa-miR-24 | 7975 | 15 | 0 |
| 825 | 8 | 90 | 388 | hsa-miR-25 | 962 | 15 | 5.9e−038 |
| 826 | 8 | 256 | 441, 487 | hsa-miR-26a | 8757 | 15 | 0 |
| 827 | 8 | 257 | 409 | hsa-miR-26b | 1364 | 15 | 0 |
| 828 | 8 | 259 | 481 | hsa-miR-27a | 6297 | 15 | 0 |
| 829 | 8 | 260 | 280 | hsa-miR-27b | 1645 | 15 | 0 |
| 830 | 8 | 269 | 362 | hsa-miR-302c* | 617 | 15 | 0 |
| 831 | 8 | 117 | 287 | hsa-miR-30a-3p | 1052 | 15 | 0 |
| 832 | 8 | 242 | 311 | hsa-miR-30b | 1168 | 15 | 0 |
| 833 | 8 | 243 | 299, 316 | hsa-miR-30c | 2096 | 15 | 0 |
| 834 | 8 | 241 | 550 | hsa-miR-30d | 2029 | 15 | 0 |
| 835 | 8 | 116 | 431 | hsa-miR-30e-3p | 685 | 15 | 0 |
| 836 | 8 | 245 | 431 | hsa-miR-30e-5p | 1956 | 15 | 0 |
| 837 | 8 | 132 | 429 | hsa-miR-31 | 1076 | 15 | 0 |
| 838 | 8 | 1 | 359 | hsa-miR-320 | 3070 | 15 | 0 |
| 839 | 8 | 124 | 455 | hsa-miR-323 | 598 | 15 | 0 |
| 840 | 8 | 91 | 368 | hsa-miR-324-3p | 783 | 15 | 0 |
| 841 | 8 | 98 | 368 | hsa-miR-324-5p | 979 | 15 | 0 |
| 842 | 8 | 113 | 538 | hsa-miR-328 | 1683 | 15 | 0 |
| 843 | 8 | 141 | 437 | hsa-miR-33 | 1673 | 15 | 0 |
| 844 | 8 | 126 | 380 | hsa-miR-331 | 924 | 15 | 0 |
| 845 | 8 | 185 | 492 | hsa-miR-335 | 739 | 15 | 0 |
| 846 | 8 | 194 | 337 | hsa-miR-338 | 516 | 15 | 0 |
| 847 | 8 | 200 | 448 | hsa-miR-339 | 511 | 15 | 0 |
| 848 | 8 | 208 | 477 | hsa-miR-342 | 2511 | 15 | 0 |
| 849 | 8 | 229 | 292 | hsa-miR-345 | 1069 | 15 | 0 |
| 850 | 8 | 237 | 480 | hsa-miR-34a | 2686 | 15 | 0 |
| 851 | 8 | 255 | 444 | hsa-miR-361 | 768 | 15 | 1.58e−038 |
| 852 | 8 | 160 | 379, 382 | hsa-miR-365 | 901 | 15 | 0 |
| 853 | 8 | 31 | 560 | hsa-miR-368 | 530 | 15 | 0 |
| 854 | 8 | 128 | 294 | hsa-miR-370 | 1030 | 15 | 0 |
| 855 | 8 | 254 | 496 | hsa-miR-374 | 742 | 15 | 0 |
| 856 | 8 | 105 | 372 | hsa-miR-378 | 527 | 15 | 0 |
| 857 | 8 | 46 | 298 | hsa-miR-383 | 533 | 15 | 0 |
| 858 | 8 | 111 | 389 | hsa-miR-422a | 595 | 15 | 0 |
| 859 | 8 | 112 | 372 | hsa-miR-422b | 925 | 15 | 0 |
| 860 | 8 | 51 | 497 | hsa-miR-423 | 3357 | 15 | 0 |
| 861 | 8 | 82 | 377 | hsa-miR-424 | 622 | 15 | 0 |
| 862 | 8 | 69 | 363 | hsa-miR-425-3p | 509 | 15 | 0 |
| 863 | 8 | 158 | 378 | hsa-miR-429 | 568 | 15 | 2.43e−038 |
| 864 | 8 | 236 | 413 | hsa-miR-449 | 4203 | 15 | 0 |
| 865 | 8 | 274 | 375, 309 | hsa-miR-450 | 548 | 15 | 0 |
| 866 | 8 | 252 | 405 | hsa-miR-452 | 809 | 15 | 0 |
| 867 | 8 | 44 | 547 | hsa-miR-485-5p | 761 | 15 | 1.49e−036 |
| 868 | 8 | 63 | 557 | hsa-miR-491 | 4511 | 15 | 0 |
| 869 | 8 | 54 | 293 | hsa-miR-492 | 1018 | 15 | 0 |
| 870 | 8 | 267 | 354 | hsa-miR-493-5p | 632 | 15 | 0 |
| 871 | 8 | 214 | 376 | hsa-miR-494 | 15470 | 15 | 0 |
| 872 | 8 | 3 | 543 | hsa-miR-495 | 502 | 15 | 0 |
| 873 | 8 | 83 | 312 | hsa-miR-497 | 1933 | 15 | 0 |
| 874 | 8 | 25 | 414 | hsa-miR-501 | 538 | 15 | 1.49e−038 |
| 875 | 8 | 43 | 418 | hsa-miR-504 | 905 | 15 | 5.53e−038 |
| 876 | 8 | 258 | 428, 443 | hsa-miR-513 | 1513 | 15 | 2.28e−038 |
| 877 | 8 | 73 | 503 | hsa-miR-518b | 632 | 15 | 0 |
| 878 | 8 | 210 | 400 | hsa-miR-518c* | 810 | 15 | 4.99e−037 |
| 879 | 8 | 262 | 523 | hsa-miR-519e* | 630 | 15 | 3.36e−038 |
| 880 | 8 | 102 | 344 | hsa-miR-520a* | 518 | 15 | 0 |
| 881 | 8 | 206 | 513 | hsa-miR-520d* | 696 | 15 | 5.82e−038 |
| 882 | 8 | 100 | 524 | hsa-miR-524* | 679 | 15 | 0 |
| 883 | 8 | 103 | 342 | hsa-miR-525 | 514 | 15 | 1.13e−037 |
| 884 | 8 | 108 | 501 | hsa-miR-526b | 543 | 15 | 1.93e−036 |
| 885 | 8 | 110 | 511, 512, 514 | hsa-miR-527 | 542 | 15 | 0 |
| 886 | 8 | 135 | 315 | hsa-miR-565 | 2609 | 13 | 6.14e−032 |
| 887 | 8 | 60 | 479 | hsa-miR-638 | 22492 | 13 | 2.23e−033 |
| 888 | 8 | 195 | 421 | hsa-miR-662 | 533 | 13 | 1.79e−035 |
| 889 | 8 | 230 | 474, 484, 536 | hsa-miR-7 | 550 | 15 | 1.9e−038 |
| 890 | 8 | 182 | 304, 317 | hsa-miR-92 | 2305 | 15 | 0 |
| 891 | 8 | 6 | 471 | hsa-miR-93 | 1687 | 15 | 0 |
| 892 | 8 | 221 | 307 | hsa-miR-98 | 521 | 15 | 0 |
| 893 | 8 | 14 | 318 | hsa-miR-99a | 3417 | 15 | 0 |
| 894 | 8 | 80 | 509 | hsa-miR-99b | 1379 | 15 | 0 |
| 895 | 8 | 131 | 430 | 6_11 | 1061 | 15 | 0 |
| 896 | 9 | 127 | 361 | 11_1 | 1374 | 6 | 8.96e−015 |
| 897 | 9 | 196 | 508 | 15_rel | 59692 | 5 | 2.09e−013 |
| 898 | 9 | 197 | 444 | 25_6 | 559 | 6 | 4.92e−016 |
| 899 | 9 | 219 | 497 | 27_3 | 2023 | 6 | 7.06e−016 |
| 900 | 9 | 42 | 449 | 43_3 | 522 | 6 | 2.3e−014 |
| 901 | 9 | 101 | 475 | 43_30 | 566 | 6 | 4.56e−018 |
| 902 | 9 | 99 | 348, 384, 434 | 43_35 | 603 | 6 | 2.1e−015 |
| 903 | 9 | 129 | 548 | 43_39 | 1750 | 6 | 2.01e−014 |
| 904 | 9 | 114 | 530 | 43_6 | 624 | 6 | 4.2e−015 |
| 905 | 9 | 183 | 373 | 66_1 | 804 | 6 | 1.5e−015 |
| 906 | 9 | 94 | 394 | 69_6_rel | 20566 | 5 | 8.09e−012 |
| 907 | 9 | 121 | 502 | 70_14 | 1006 | 5 | 1.37e−011 |
| 908 | 9 | 177 | 366, 464 | 72_12 | 2254 | 5 | 2.93e−011 |
| 909 | 9 | 40 | 553 | 72_4 | 3214 | 5 | 2.35e−012 |
| 910 | 9 | 97 | 357 | 74_26_rel | 3754 | 5 | 1.47e−012 |
| 911 | 9 | 87 | 301 | gam11 | 16548 | 5 | 3.42e−013 |
| 912 | 9 | 125 | 532 | 70_16 | 506 | 6 | 1.79e−015 |
| 913 | 9 | 163 | 402 | 43_7 | 527 | 5 | 1.35e−011 |
| 914 | 9 | 223 | 320, 393, 559 | hsa-let-7a | 11127 | 6 | 6.75e−015 |
| 915 | 9 | 225 | 332 | hsa-let-7b | 14243 | 6 | 4.01e−015 |
| 916 | 9 | 224 | 427 | hsa-let-7c | 12791 | 6 | 4.71e−015 |
| 917 | 9 | 45 | 277 | hsa-let-7d | 3804 | 6 | 3.85e−015 |
| 918 | 9 | 220 | 475 | hsa-let-7e | 4216 | 6 | 3.8e−016 |
| 919 | 9 | 222 | 482, 558 | hsa-let-7f | 1424 | 6 | 3.3e−014 |
| 920 | 9 | 226 | 395 | hsa-let-7g | 2445 | 6 | 5.57e−016 |
| 921 | 9 | 227 | 297 | hsa-let-7i | 2224 | 6 | 1.16e−016 |
| 922 | 9 | 231 | 321, 424 | hsa-miR-1 | 548 | 6 | 2.54e−015 |
| 923 | 9 | 13 | 397 | hsa-miR-100 | 1184 | 6 | 3.05e−014 |
| 924 | 9 | 48 | 483, 529 | hsa-miR-103 | 2535 | 6 | 5.58e−016 |
| 925 | 9 | 184 | 423 | hsa-miR-105 | 1589 | 6 | 2.67e−016 |
| 926 | 9 | 2 | 399 | hsa-miR-106a | 2394 | 6 | 8.92e−015 |
| 927 | 9 | 149 | 392 | hsa-miR-106b | 1735 | 6 | 4.45e−016 |
| 928 | 9 | 47 | 555 | hsa-miR-107 | 2691 | 6 | 8.53e−016 |
| 929 | 9 | 199 | 387 | hsa-miR-125a | 2859 | 6 | 2.03e−015 |
| 930 | 9 | 198 | 313, 551 | hsa-miR-125b | 5141 | 6 | 3.92e−015 |
| 931 | 9 | 204 | 419 | hsa-miR-126 | 2095 | 6 | 8.8e−015 |
| 932 | 9 | 89 | 419 | hsa-miR-126* | 879 | 6 | 6.63e−017 |
| 933 | 9 | 86 | 417 | hsa-miR-130a | 3652 | 6 | 2.01e−015 |
| 934 | 9 | 85 | 328 | hsa-miR-130b | 614 | 6 | 5.83e−014 |
| 935 | 9 | 181 | 324, 554 | hsa-miR-135a | 530 | 6 | 2.77e−014 |
| 936 | 9 | 180 | 486 | hsa-miR-135b | 520 | 6 | 2.46e−014 |
| 937 | 9 | 37 | 391 | hsa-miR-136 | 695 | 6 | 2.56e−015 |
| 938 | 9 | 52 | 370, 548 | hsa-miR-138 | 810 | 6 | 2.58e−016 |
| 939 | 9 | 64 | 402 | hsa-miR-140 | 1068 | 6 | 8.12e−016 |
| 940 | 9 | 151 | 472 | hsa-miR-141 | 3688 | 6 | 2.73e−015 |
| 941 | 9 | 218 | 527 | hsa-miR-143 | 3292 | 6 | 1.01e−014 |
| 942 | 9 | 139 | 281 | hsa-miR-145 | 2405 | 6 | 1.95e−014 |
| 943 | 9 | 217 | 466 | hsa-miR-146a | 555 | 6 | 3.4e−017 |
| 944 | 9 | 146 | 325 | hsa-miR-147 | 722 | 6 | 1.96e−015 |
| 945 | 9 | 190 | 458 | hsa-miR-148a | 701 | 6 | 7.05e−015 |
| 946 | 9 | 191 | 289 | hsa-miR-148b | 843 | 6 | 5.89e−018 |
| 947 | 9 | 212 | 333 | hsa-miR-149 | 1188 | 6 | 4.17e−015 |
| 948 | 9 | 209 | 507 | hsa-miR-150 | 839 | 6 | 3.14e−014 |
| 949 | 9 | 34 | 396 | hsa-miR-151 | 738 | 6 | 3.18e−017 |
| 950 | 9 | 192 | 470 | hsa-miR-152 | 627 | 6 | 1.55e−016 |
| 951 | 9 | 263 | 296, 404 | hsa-miR-153 | 539 | 6 | 2.82e−015 |
| 952 | 9 | 253 | 499 | hsa-miR-155 | 593 | 6 | 7.08e−017 |
| 953 | 9 | 171 | 331 | hsa-miR-15a | 824 | 6 | 5.17e−016 |
| 954 | 9 | 172 | 329 | hsa-miR-15b | 1969 | 6 | 5.6e−015 |
| 955 | 9 | 173 | 360, 468 | hsa-miR-16 | 3825 | 6 | 7.06e−015 |
| 956 | 9 | 38 | 467 | hsa-miR-17-3p | 870 | 6 | 1.74e−015 |
| 957 | 9 | 75 | 467 | hsa-miR-17-5p | 2169 | 6 | 1.28e−014 |
| 958 | 9 | 10 | 371, 461 | hsa-miR-181a | 3443 | 6 | 7.49e−014 |
| 959 | 9 | 11 | 456, 540 | hsa-miR-181b | 1010 | 6 | 6.43e−014 |
| 960 | 9 | 9 | 451 | hsa-miR-181c | 517 | 6 | 3.38e−015 |
| 961 | 9 | 272 | 323 | hsa-miR-182 | 502 | 6 | 2.37e−014 |
| 962 | 9 | 179 | 356 | hsa-miR-183 | 1767 | 6 | 1.66e−014 |
| 963 | 9 | 234 | 450 | hsa-miR-185 | 866 | 6 | 7.91e−018 |
| 964 | 9 | 144 | 346 | hsa-miR-189 | 704 | 6 | 4.62e−015 |
| 965 | 9 | 155 | 426 | hsa-miR-18a | 534 | 6 | 2.36e−015 |
| 966 | 9 | 77 | 440 | hsa-miR-191 | 2519 | 6 | 6.1e−016 |
| 967 | 9 | 17 | 442 | hsa-miR-193a | 4711 | 6 | 3.26e−015 |
| 968 | 9 | 16 | 488 | hsa-miR-193b | 1971 | 6 | 1.46e−014 |
| 969 | 9 | 170 | 327 | hsa-miR-195 | 1319 | 6 | 1.98e−014 |
| 970 | 9 | 136 | 308 | hsa-miR-198 | 583 | 6 | 5.31e−015 |
| 971 | 9 | 92 | 410, 432 | hsa-miR-199a | 4621 | 6 | 1.62e−014 |
| 972 | 9 | 162 | 410, 432, 473 | hsa-miR-199a* | 1754 | 6 | 2.98e−013 |
| 973 | 9 | 93 | 473 | hsa-miR-199b | 680 | 6 | 1.82e−014 |
| 974 | 9 | 251 | 282, 495 | hsa-miR-19b | 2158 | 6 | 5.2e−014 |
| 975 | 9 | 152 | 369 | hsa-miR-200a | 1176 | 6 | 2.59e−015 |
| 976 | 9 | 157 | 411 | hsa-miR-200b | 2359 | 6 | 1.28e−014 |
| 977 | 9 | 156 | 446 | hsa-miR-200c | 5736 | 6 | 4.25e−015 |
| 978 | 9 | 271 | 334 | hsa-miR-202* | 705 | 6 | 4.67e−014 |
| 979 | 9 | 203 | 504 | hsa-miR-205 | 4306 | 6 | 2.29e−013 |
| 980 | 9 | 232 | 422 | hsa-miR-206 | 771 | 6 | 2.08e−015 |
| 981 | 9 | 150 | 351 | hsa-miR-20a | 729 | 6 | 2.73e−014 |
| 982 | 9 | 175 | 462 | hsa-miR-21 | 5568 | 6 | 5.04e−015 |
| 983 | 9 | 115 | 314 | hsa-miR-210 | 608 | 6 | 3.65e−014 |
| 984 | 9 | 29 | 439 | hsa-miR-214 | 2521 | 6 | 2.59e−015 |
| 985 | 9 | 159 | 494 | hsa-miR-216 | 824 | 6 | 1.4e−015 |
| 986 | 9 | 268 | 401, 425 | hsa-miR-218 | 596 | 6 | 3.42e−015 |
| 987 | 9 | 18 | 322 | hsa-miR-22 | 1863 | 6 | 8.57e−016 |
| 988 | 9 | 50 | 364 | hsa-miR-221 | 1124 | 6 | 5.54e−016 |
| 989 | 9 | 49 | 302 | hsa-miR-222 | 1653 | 6 | 5.23e−017 |
| 990 | 9 | 248 | 415 | hsa-miR-223 | 535 | 6 | 4.54e−015 |
| 991 | 9 | 67 | 336 | hsa-miR-23a | 3156 | 6 | 1.98e−015 |
| 992 | 9 | 238 | 346, 416 | hsa-miR-24 | 6493 | 6 | 2.19e−016 |
| 993 | 9 | 90 | 388 | hsa-miR-25 | 1139 | 6 | 6.25e−016 |
| 994 | 9 | 256 | 441, 487 | hsa-miR-26a | 7026 | 6 | 1.41e−016 |
| 995 | 9 | 257 | 409 | hsa-miR-26b | 1338 | 6 | 1.45e−015 |
| 996 | 9 | 259 | 481 | hsa-miR-27a | 4529 | 6 | 1.05e−015 |
| 997 | 9 | 260 | 280 | hsa-miR-27b | 1377 | 6 | 2.87e−015 |
| 998 | 9 | 61 | 286 | hsa-miR-296 | 573 | 6 | 1.15e−012 |
| 999 | 9 | 269 | 362 | hsa-miR-302c* | 596 | 6 | 1.58e−015 |
| 1000 | 9 | 117 | 287 | hsa-miR-30a-3p | 1098 | 6 | 3.75e−016 |
| 1001 | 9 | 242 | 311 | hsa-miR-30b | 1170 | 6 | 6.47e−015 |
| 1002 | 9 | 243 | 299, 316 | hsa-miR-30c | 2051 | 6 | 2.87e−014 |
| 1003 | 9 | 241 | 550 | hsa-miR-30d | 1800 | 6 | 6.15e−016 |
| 1004 | 9 | 116 | 431 | hsa-miR-30e-3p | 769 | 6 | 1.2e−016 |
| 1005 | 9 | 245 | 431 | hsa-miR-30e-5p | 1732 | 6 | 1.41e−016 |
| 1006 | 9 | 132 | 429 | hsa-miR-31 | 1017 | 6 | 4.51e−017 |
| 1007 | 9 | 1 | 359 | hsa-miR-320 | 2846 | 6 | 3.56e−017 |
| 1008 | 9 | 124 | 455 | hsa-miR-323 | 538 | 6 | 2.2e−014 |
| 1009 | 9 | 91 | 368 | hsa-miR-324-3p | 647 | 6 | 1.18e−015 |
| 1010 | 9 | 98 | 368 | hsa-miR-324-5p | 874 | 6 | 1.06e−015 |
| 1011 | 9 | 113 | 538 | hsa-miR-328 | 1497 | 6 | 4.46e−016 |
| 1012 | 9 | 141 | 437 | hsa-miR-331 | 1494 | 6 | 6.41e−015 |
| 1013 | 9 | 126 | 380 | hsa-miR-331 | 1027 | 6 | 5.99e−016 |
| 1014 | 9 | 185 | 492 | hsa-miR-335 | 912 | 6 | 2.87e−015 |
| 1015 | 9 | 200 | 448 | hsa-miR-339 | 625 | 6 | 1.04e−015 |
| 1016 | 9 | 208 | 477 | hsa-miR-342 | 2039 | 6 | 2.72e−014 |
| 1017 | 9 | 229 | 292 | hsa-miR-345 | 1046 | 6 | 3.27e−015 |
| 1018 | 9 | 237 | 480 | hsa-miR-34a | 2220 | 6 | 3.92e−016 |
| 1019 | 9 | 255 | 444 | hsa-miR-361 | 835 | 6 | 2.64e−016 |
| 1020 | 9 | 160 | 379, 382 | hsa-miR-365 | 753 | 6 | 6.54e−015 |
| 1021 | 9 | 128 | 294 | hsa-miR-370 | 945 | 6 | 3.83e−015 |
| 1022 | 9 | 35 | 406 | hsa-miR-373* | 884 | 6 | 1.25e−017 |
| 1023 | 9 | 254 | 496 | hsa-miR-374 | 730 | 6 | 1.91e−015 |
| 1024 | 9 | 105 | 372 | hsa-miR-378 | 519 | 6 | 5.48e−016 |
| 1025 | 9 | 111 | 389 | hsa-miR-422a | 705 | 6 | 6.41e−017 |
| 1026 | 9 | 112 | 372 | hsa-miR-422b | 881 | 6 | 3.88e−017 |
| 1027 | 9 | 51 | 497 | hsa-miR-423 | 3011 | 6 | 7.66e−016 |
| 1028 | 9 | 82 | 377 | hsa-miR-424 | 671 | 6 | 2.22e−018 |
| 1029 | 9 | 236 | 413 | hsa-miR-449 | 3698 | 6 | 3.02e−014 |
| 1030 | 9 | 274 | 375, 309 | hsa-miR-450 | 557 | 6 | 2.55e−015 |
| 1031 | 9 | 252 | 405 | hsa-miR-452 | 909 | 6 | 7.39e−016 |
| 1032 | 9 | 44 | 547 | hsa-miR-485-5p | 940 | 6 | 6.19e−015 |
| 1033 | 9 | 63 | 557 | hsa-miR-491 | 5160 | 6 | 7.68e−016 |
| 1034 | 9 | 54 | 293 | hsa-miR-492 | 1032 | 6 | 4.38e−015 |
| 1035 | 9 | 267 | 354 | hsa-miR-493-5p | 565 | 6 | 3.31e−015 |
| 1036 | 9 | 214 | 376 | hsa-miR-494 | 15838 | 6 | 6.43e−015 |
| 1037 | 9 | 83 | 312 | hsa-miR-497 | 1528 | 6 | 2.77e−015 |
| 1038 | 9 | 43 | 418 | hsa-miR-504 | 786 | 6 | 3.5e−014 |
| 1039 | 9 | 258 | 428, 443 | hsa-miR-513 | 1222 | 6 | 3.75e−014 |
| 1040 | 9 | 73 | 503 | hsa-miR-518b | 625 | 6 | 1.07e−015 |
| 1041 | 9 | 210 | 400 | hsa-miR-518c* | 698 | 6 | 5.01e−014 |
| 1042 | 9 | 262 | 523 | hsa-miR-519e* | 565 | 6 | 1.62e−014 |
| 1043 | 9 | 206 | 513 | hsa-miR-520d* | 568 | 6 | 1.74e−014 |
| 1044 | 9 | 100 | 524 | hsa-miR-524* | 613 | 6 | 1.62e−014 |
| 1045 | 9 | 108 | 501 | hsa-miR-526b | 563 | 6 | 2.74e−014 |
| 1046 | 9 | 135 | 315 | hsa-miR-565 | 1737 | 5 | 1.16e−010 |
| 1047 | 9 | 60 | 479 | hsa-miR-638 | 22676 | 5 | 2.27e−011 |
| 1048 | 9 | 230 | 474, 484, 536 | hsa-miR-7 | 564 | 6 | 3.15e−014 |
| 1049 | 9 | 182 | 304, 317 | hsa-miR-92 | 2612 | 6 | 1.35e−014 |
| 1050 | 9 | 6 | 471 | hsa-miR-93 | 1556 | 6 | 9.85e−016 |
| 1051 | 9 | 221 | 307 | hsa-miR-98 | 544 | 6 | 1.48e−017 |
| 1052 | 9 | 14 | 318 | hsa-miR-99a | 2506 | 6 | 1.69e−014 |
| 1053 | 9 | 80 | 509 | hsa-miR-99b | 1383 | 6 | 6.18e−016 |
| 1054 | 9 | 131 | 430 | 6_11 | 990 | 6 | 4.42e−015 |
| 1055 | 9 | 23 | 546 | 47_6 | 623 | 6 | 1.76e−014 |
| 1056 | 10 | 127 | 361 | 11_1 | 981 | 7 | 4.39e−021 |
| 1057 | 10 | 196 | 508 | 15_rel | 61157 | 6 | 4.5e−017 |
| 1058 | 10 | 197 | 444 | 25_6 | 653 | 7 | 5.21e−019 |
| 1059 | 10 | 219 | 497 | 27_3 | 1787 | 7 | 1.62e−019 |
| 1060 | 10 | 101 | 475 | 43_30 | 543 | 7 | 8.47e−018 |
| 1061 | 10 | 129 | 548 | 43_39 | 1762 | 7 | 1.87e−017 |
| 1062 | 10 | 114 | 530 | 43_6 | 517 | 7 | 1.09e−017 |
| 1063 | 10 | 183 | 373 | 66_1 | 679 | 7 | 3.19e−017 |
| 1064 | 10 | 94 | 394 | 69_6_rel | 17459 | 6 | 9.14e−015 |
| 1065 | 10 | 121 | 502 | 70_14 | 962 | 6 | 4.86e−017 |
| 1066 | 10 | 177 | 366, 464 | 72_12 | 2267 | 6 | 1.96e−015 |
| 1067 | 10 | 40 | 553 | 72_4 | 2468 | 6 | 3.71e−015 |
| 1068 | 10 | 97 | 357 | 74_26_rel | 4293 | 6 | 2.26e−016 |
| 1069 | 10 | 87 | 301 | gam11 | 25052 | 6 | 2.01e−016 |
| 1070 | 10 | 125 | 532 | 70_16 | 529 | 7 | 4.77e−018 |
| 1071 | 10 | 163 | 402 | 43_7 | 815 | 6 | 7.65e−015 |
| 1072 | 10 | 223 | 320, 393, 559 | hsa-let-7a | 12900 | 7 | 1.14e−018 |
| 1073 | 10 | 225 | 332 | hsa-let-7b | 27951 | 7 | 4.95e−019 |
| 1074 | 10 | 224 | 427 | hsa-let-7c | 20368 | 7 | 1.97e−019 |
| 1075 | 10 | 45 | 277 | hsa-let-7d | 3367 | 7 | 2.72e−019 |
| 1076 | 10 | 220 | 475 | hsa-let-7e | 3880 | 7 | 1.96e−021 |
| 1077 | 10 | 222 | 482, 558 | hsa-let-7f | 989 | 7 | 1.96e−018 |
| 1078 | 10 | 226 | 395 | hsa-let-7g | 2057 | 7 | 1.95e−019 |
| 1079 | 10 | 227 | 297 | hsa-let-7i | 2391 | 7 | 1.69e−020 |
| 1080 | 10 | 13 | 397 | hsa-miR-100 | 1473 | 7 | 5.46e−019 |
| 1081 | 10 | 48 | 483, 529 | hsa-miR-103 | 2671 | 7 | 6.84e−019 |
| 1082 | 10 | 184 | 423 | hsa-miR-105 | 1464 | 7 | 8.05e−022 |
| 1083 | 10 | 2 | 399 | hsa-miR-106a | 1789 | 7 | 1.76e−020 |
| 1084 | 10 | 149 | 392 | hsa-miR-106b | 1776 | 7 | 1.24e−022 |
| 1085 | 10 | 47 | 555 | hsa-miR-107 | 2573 | 7 | 1.28e−019 |
| 1086 | 10 | 199 | 387 | hsa-miR-125a | 2861 | 7 | 1.83e−019 |
| 1087 | 10 | 198 | 313, 551 | hsa-miR-125b | 7585 | 7 | 4.95e−018 |
| 1088 | 10 | 204 | 419 | hsa-miR-126 | 3129 | 7 | 7.9e−019 |
| 1089 | 10 | 89 | 419 | hsa-miR-126* | 731 | 7 | 1.68e−017 |
| 1090 | 10 | 86 | 417 | hsa-miR-130a | 3105 | 7 | 8.25e−020 |
| 1091 | 10 | 181 | 324, 554 | hsa-miR-135a | 501 | 7 | 4.73e−017 |
| 1092 | 10 | 180 | 486 | hsa-miR-135b | 500 | 7 | 5.3e−017 |
| 1093 | 10 | 37 | 391 | hsa-miR-136 | 539 | 7 | 7.08e−017 |
| 1094 | 10 | 52 | 370, 548 | hsa-miR-138 | 544 | 7 | 6.97e−017 |
| 1095 | 10 | 64 | 402 | hsa-miR-140 | 1022 | 7 | 9.56e−018 |
| 1096 | 10 | 151 | 472 | hsa-miR-141 | 4758 | 7 | 6.7e−019 |
| 1097 | 10 | 218 | 527 | hsa-miR-143 | 5733 | 7 | 5.85e−019 |
| 1098 | 10 | 139 | 281 | hsa-miR-145 | 4519 | 7 | 2.53e−018 |
| 1099 | 10 | 146 | 325 | hsa-miR-147 | 504 | 7 | 1.85e−017 |
| 1100 | 10 | 190 | 458 | hsa-miR-148a | 655 | 7 | 3.25e−018 |
| 1101 | 10 | 191 | 289 | hsa-miR-148b | 685 | 7 | 4.66e−022 |
| 1102 | 10 | 212 | 333 | hsa-miR-149 | 1262 | 7 | 3.5e−019 |
| 1103 | 10 | 209 | 507 | hsa-miR-150 | 730 | 7 | 2.85e−019 |
| 1104 | 10 | 34 | 396 | hsa-miR-151 | 685 | 7 | 1.22e−019 |
| 1105 | 10 | 192 | 470 | hsa-miR-152 | 803 | 7 | 3.14e−021 |
| 1106 | 10 | 171 | 331 | hsa-miR-15a | 730 | 7 | 8.52e−021 |
| 1107 | 10 | 172 | 329 | hsa-miR-15b | 1592 | 7 | 1.16e−019 |
| 1108 | 10 | 173 | 360, 468 | hsa-miR-16 | 5751 | 7 | 1.62e−019 |
| 1109 | 10 | 38 | 467 | hsa-miR-17-3p | 686 | 7 | 1.15e−022 |
| 1110 | 10 | 75 | 467 | hsa-miR-17-5p | 1470 | 7 | 2.92e−020 |
| 1111 | 10 | 10 | 371, 461 | hsa-miR-181a | 2551 | 7 | 1.07e−019 |
| 1112 | 10 | 11 | 456, 540 | hsa-miR-181b | 611 | 7 | 2.18e−018 |
| 1113 | 10 | 9 | 451 | hsa-miR-181c | 519 | 7 | 5.19e−018 |
| 1114 | 10 | 272 | 323 | hsa-miR-182 | 705 | 7 | 3.02e−017 |
| 1115 | 10 | 179 | 356 | hsa-miR-183 | 1655 | 7 | 1.6e−016 |
| 1116 | 10 | 234 | 450 | hsa-miR-185 | 926 | 7 | 2.04e−021 |
| 1117 | 10 | 88 | 310 | hsa-miR-188 | 539 | 7 | 5.83e−017 |
| 1118 | 10 | 144 | 346 | hsa-miR-189 | 634 | 7 | 1.06e−017 |
| 1119 | 10 | 77 | 440 | hsa-miR-191 | 3191 | 7 | 8.7e−019 |
| 1120 | 10 | 130 | 440 | hsa-miR-191* | 742 | 6 | 1.82e−017 |
| 1121 | 10 | 17 | 442 | hsa-miR-193a | 5827 | 7 | 6.88e−020 |
| 1122 | 10 | 16 | 488 | hsa-miR-193b | 1961 | 7 | 1.65e−018 |
| 1123 | 10 | 170 | 327 | hsa-miR-195 | 1599 | 7 | 1.05e−018 |
| 1124 | 10 | 136 | 308 | hsa-miR-198 | 526 | 7 | 5.35e−017 |
| 1125 | 10 | 92 | 410, 432 | hsa-miR-199a | 5529 | 7 | 8.5e−019 |
| 1126 | 10 | 162 | 410, 432, 473 | hsa-miR-199a* | 1306 | 7 | 2.44e−016 |
| 1127 | 10 | 93 | 473 | hsa-miR-199b | 718 | 7 | 3.56e−017 |
| 1128 | 10 | 251 | 282, 495 | hsa-miR-19b | 1569 | 7 | 1.85e−018 |
| 1129 | 10 | 152 | 369 | hsa-miR-200a | 1545 | 7 | 3.92e−018 |
| 1130 | 10 | 157 | 411 | hsa-miR-200b | 2352 | 7 | 3.11e−019 |
| 1131 | 10 | 156 | 446 | hsa-miR-200c | 4932 | 7 | 9.15e−019 |
| 1132 | 10 | 271 | 334 | hsa-miR-202* | 745 | 7 | 3.34e−017 |
| 1133 | 10 | 203 | 504 | hsa-miR-205 | 3997 | 7 | 2.78e−016 |
| 1134 | 10 | 150 | 351 | hsa-miR-20a | 518 | 7 | 1.57e−018 |
| 1135 | 10 | 175 | 462 | hsa-miR-21 | 9427 | 7 | 3.59e−017 |
| 1136 | 10 | 115 | 314 | hsa-miR-210 | 630 | 7 | 9.03e−018 |
| 1137 | 10 | 29 | 439 | hsa-miR-214 | 2701 | 7 | 1.48e−019 |
| 1138 | 10 | 159 | 494 | hsa-miR-216 | 599 | 7 | 3.75e−017 |
| 1139 | 10 | 18 | 322 | hsa-miR-22 | 3665 | 7 | 7.51e−019 |
| 1140 | 10 | 50 | 364 | hsa-miR-221 | 2214 | 7 | 2.58e−018 |
| 1141 | 10 | 49 | 302 | hsa-miR-222 | 3240 | 7 | 7.72e−019 |
| 1142 | 10 | 248 | 415 | hsa-miR-223 | 625 | 7 | 9.55e−018 |
| 1143 | 10 | 67 | 336 | hsa-miR-23a | 5406 | 7 | 5.72e−019 |
| 1144 | 10 | 238 | 346, 416 | hsa-miR-24 | 10063 | 7 | 3.68e−019 |
| 1145 | 10 | 90 | 388 | hsa-miR-25 | 1146 | 7 | 1.54e−020 |
| 1146 | 10 | 256 | 441, 487 | hsa-miR-26a | 9694 | 7 | 3.41e−020 |
| 1147 | 10 | 257 | 409 | hsa-miR-26b | 1131 | 7 | 2.33e−018 |
| 1148 | 10 | 259 | 481 | hsa-miR-27a | 7988 | 7 | 2.38e−019 |
| 1149 | 10 | 260 | 280 | hsa-miR-27b | 1885 | 7 | 2.15e−018 |
| 1150 | 10 | 269 | 362 | hsa-miR-302c* | 507 | 7 | 2.73e−017 |
| 1151 | 10 | 117 | 287 | hsa-miR-30a-3p | 915 | 7 | 5.38e−020 |
| 1152 | 10 | 242 | 311 | hsa-miR-30b | 1155 | 7 | 8.15e−018 |
| 1153 | 10 | 243 | 299, 316 | hsa-miR-30c | 2133 | 7 | 2e−018 |
| 1154 | 10 | 241 | 550 | hsa-miR-30d | 2150 | 7 | 5.1e−019 |
| 1155 | 10 | 116 | 431 | hsa-miR-30e-3p | 529 | 7 | 6.63e−018 |
| 1156 | 10 | 245 | 431 | hsa-miR-30e-5p | 1998 | 7 | 6.66e−019 |
| 1157 | 10 | 132 | 429 | hsa-miR-31 | 976 | 7 | 1.26e−021 |
| 1158 | 10 | 1 | 359 | hsa-miR-320 | 3095 | 7 | 1.27e−020 |
| 1159 | 10 | 124 | 455 | hsa-miR-323 | 520 | 7 | 4.8e−018 |
| 1160 | 10 | 91 | 368 | hsa-miR-324-3p | 874 | 7 | 4.51e−020 |
| 1161 | 10 | 98 | 368 | hsa-miR-324-5p | 912 | 7 | 4.36e−018 |
| 1162 | 10 | 113 | 538 | hsa-miR-328 | 1584 | 7 | 3.51e−018 |
| 1163 | 10 | 141 | 437 | hsa-miR-33 | 1497 | 7 | 1.84e−017 |
| 1164 | 10 | 126 | 380 | hsa-miR-331 | 859 | 7 | 3.8e−020 |
| 1165 | 10 | 208 | 477 | hsa-miR-342 | 2666 | 7 | 2.34e−019 |
| 1166 | 10 | 229 | 292 | hsa-miR-345 | 855 | 7 | 3.76e−017 |
| 1167 | 10 | 237 | 480 | hsa-miR-34a | 2976 | 7 | 2.76e−022 |
| 1168 | 10 | 255 | 444 | hsa-miR-361 | 910 | 7 | 3.38e−020 |
| 1169 | 10 | 160 | 379, 382 | hsa-miR-365 | 904 | 7 | 6.2e−018 |
| 1170 | 10 | 128 | 294 | hsa-miR-370 | 861 | 7 | 5.22e−017 |
| 1171 | 10 | 254 | 496 | hsa-miR-374 | 602 | 7 | 1.27e−017 |
| 1172 | 10 | 273 | 335 | hsa-miR-375 | 578 | 7 | 1.12e−016 |
| 1173 | 10 | 112 | 372 | hsa-miR-422b | 769 | 7 | 8.32e−020 |
| 1174 | 10 | 51 | 497 | hsa-miR-423 | 3238 | 7 | 6.08e−020 |
| 1175 | 10 | 158 | 378 | hsa-miR-429 | 563 | 7 | 6.97e−017 |
| 1176 | 10 | 236 | 413 | hsa-miR-449 | 3902 | 7 | 2.07e−018 |
| 1177 | 10 | 252 | 405 | hsa-miR-452 | 573 | 7 | 5.3e−017 |
| 1178 | 10 | 44 | 547 | hsa-miR-485-5p | 769 | 7 | 8.27e−017 |
| 1179 | 10 | 63 | 557 | hsa-miR-491 | 3379 | 7 | 7.52e−018 |
| 1180 | 10 | 54 | 293 | hsa-miR-492 | 817 | 7 | 1.42e−017 |
| 1181 | 10 | 267 | 354 | hsa-miR-493-5p | 540 | 7 | 9.14e−019 |
| 1182 | 10 | 214 | 376 | hsa-miR-494 | 15562 | 7 | 5.37e−019 |
| 1183 | 10 | 83 | 312 | hsa-miR-497 | 2207 | 7 | 2.1e−020 |
| 1184 | 10 | 43 | 418 | hsa-miR-504 | 792 | 7 | 3.98e−017 |
| 1185 | 10 | 258 | 428, 443 | hsa-miR-513 | 1410 | 7 | 2.56e−017 |
| 1186 | 10 | 73 | 503 | hsa-miR-518b | 526 | 7 | 2.69e−017 |
| 1187 | 10 | 210 | 400 | hsa-miR-518c* | 673 | 7 | 1.42e−016 |
| 1188 | 10 | 262 | 523 | hsa-miR-519e* | 524 | 7 | 5.98e−017 |
| 1189 | 10 | 206 | 513 | hsa-miR-520d* | 650 | 7 | 7.91e−017 |
| 1190 | 10 | 100 | 524 | hsa-miR-524* | 567 | 7 | 2.59e−017 |
| 1191 | 10 | 103 | 342 | hsa-miR-525 | 618 | 7 | 3.67e−018 |
| 1192 | 10 | 108 | 501 | hsa-miR-526b | 571 | 7 | 7.77e−017 |
| 1193 | 10 | 56 | 338 | hsa-miR-564 | 554 | 6 | 7.3e−015 |
| 1194 | 10 | 135 | 315 | hsa-miR-565 | 3482 | 6 | 1.57e−016 |
| 1195 | 10 | 60 | 479 | hsa-miR-638 | 21557 | 6 | 5.71e−015 |
| 1196 | 10 | 195 | 421 | hsa-miR-662 | 655 | 6 | 3.15e−016 |
| 1197 | 10 | 182 | 304, 317 | hsa-miR-92 | 2098 | 7 | 1.68e−019 |
| 1198 | 10 | 6 | 471 | hsa-miR-93 | 1673 | 7 | 1.12e−020 |
| 1199 | 10 | 14 | 318 | hsa-miR-99a | 3636 | 7 | 2.24e−018 |
| 1200 | 10 | 80 | 509 | hsa-miR-99b | 1351 | 7 | 3.36e−021 |
| 1201 | 10 | 131 | 430 | 6_11 | 1015 | 7 | 5.35e−018 |
| 1202 | 11 | 127 | 361 | 11_1 | 715 | 5 | 9.44e−010 |
| 1203 | 11 | 196 | 508 | 15_rel | 32621 | 5 | 1.1e−010 |
| 1204 | 11 | 197 | 444 | 25_6 | 505 | 5 | 1.09e−011 |
| 1205 | 11 | 219 | 497 | 27_3 | 1201 | 5 | 2.07e−010 |
| 1206 | 11 | 27 | 313 | 43_40 | 662 | 5 | 1.1e−012 |
| 1207 | 11 | 145 | 447 | 43_44_rel | 38489 | 5 | 7.36e−011 |
| 1208 | 11 | 94 | 394 | 69_6_rel | 5702 | 5 | 3.36e−010 |
| 1209 | 11 | 177 | 366, 464 | 72_12 | 1224 | 5 | 1.06e−010 |
| 1210 | 11 | 40 | 553 | 72_4 | 612 | 5 | 1.57e−009 |
| 1211 | 11 | 97 | 357 | 74_26_rel | 1504 | 5 | 3.69e−010 |
| 1212 | 11 | 33 | 339, 552 | 96_rel | 44437 | 5 | 2.39e−012 |
| 1213 | 11 | 87 | 301 | gam11 | 16445 | 5 | 3.41e−011 |
| 1214 | 11 | 223 | 320, 393, 559 | hsa-let-7a | 3956 | 5 | 1.16e−009 |
| 1215 | 11 | 223 | 320, 393, 559 | hsa-let-7a | 957 | 5 | 1.41e−008 |
| 1216 | 11 | 225 | 332 | hsa-let-7b | 12620 | 5 | 7.1e−010 |
| 1217 | 11 | 224 | 427 | hsa-let-7c | 10477 | 5 | 1.47e−010 |
| 1218 | 11 | 45 | 277 | hsa-let-7d | 1394 | 5 | 2.79e−010 |
| 1219 | 11 | 220 | 475 | hsa-let-7e | 1791 | 5 | 4.22e−012 |
| 1220 | 11 | 226 | 395 | hsa-let-7g | 977 | 5 | 2.34e−010 |
| 1221 | 11 | 227 | 297 | hsa-let-7i | 1478 | 5 | 2.6e−011 |
| 1222 | 11 | 13 | 397 | hsa-miR-100 | 1429 | 5 | 7.36e−011 |
| 1223 | 11 | 48 | 483, 529 | hsa-miR-103 | 1090 | 5 | 1.12e−009 |
| 1224 | 11 | 184 | 423 | hsa-miR-105 | 640 | 5 | 1.99e−010 |
| 1225 | 11 | 2 | 399 | hsa-miR-106a | 530 | 5 | 6.33e−009 |
| 1226 | 11 | 149 | 392 | hsa-miR-106b | 1164 | 5 | 1.05e−011 |
| 1227 | 11 | 47 | 555 | hsa-miR-107 | 1103 | 5 | 2.99e−010 |
| 1228 | 11 | 199 | 387 | hsa-miR-125a | 3719 | 5 | 2.08e−012 |
| 1229 | 11 | 198 | 313, 551 | hsa-miR-125b | 17469 | 5 | 4.46e−012 |
| 1230 | 11 | 204 | 419 | hsa-miR-126 | 1450 | 5 | 1.25e−009 |
| 1231 | 11 | 89 | 419 | hsa-miR-126* | 579 | 5 | 2.7e−010 |
| 1232 | 11 | 86 | 417 | hsa-miR-130a | 2513 | 5 | 2.68e−011 |
| 1233 | 11 | 64 | 402 | hsa-miR-140 | 607 | 5 | 1.04e−010 |
| 1234 | 11 | 151 | 472 | hsa-miR-141 | 3018 | 5 | 4.86e−011 |
| 1235 | 11 | 218 | 527 | hsa-miR-143 | 12739 | 5 | 8.41e−010 |
| 1236 | 11 | 139 | 281 | hsa-miR-145 | 11223 | 5 | 9.3e−012 |
| 1237 | 11 | 212 | 333 | hsa-miR-149 | 558 | 5 | 1.56e−009 |
| 1238 | 11 | 209 | 507 | hsa-miR-150 | 697 | 5 | 7.44e−011 |
| 1239 | 11 | 173 | 360, 468 | hsa-miR-16 | 2005 | 5 | 1.22e−008 |
| 1240 | 11 | 75 | 467 | hsa-miR-17-5p | 529 | 5 | 1.93e−009 |
| 1241 | 11 | 10 | 371, 461 | hsa-miR-181a | 1755 | 5 | 6.68e−012 |
| 1242 | 11 | 179 | 356 | hsa-miR-183 | 928 | 5 | 5.76e−010 |
| 1243 | 11 | 77 | 440 | hsa-miR-191 | 853 | 5 | 1.59e−010 |
| 1244 | 11 | 130 | 440 | hsa-miR-191* | 843 | 5 | 1.43e−010 |
| 1245 | 11 | 17 | 442 | hsa-miR-193a | 1259 | 5 | 1.51e−009 |
| 1246 | 11 | 16 | 488 | hsa-miR-193b | 1257 | 5 | 1.08e−010 |
| 1247 | 11 | 170 | 327 | hsa-miR-195 | 502 | 5 | 4.64e−009 |
| 1248 | 11 | 92 | 410, 432 | hsa-miR-199a | 5849 | 5 | 9.31e−010 |
| 1249 | 11 | 162 | 410, 432, 473 | hsa-miR-199a* | 731 | 5 | 7.85e−009 |
| 1250 | 11 | 251 | 282, 495 | hsa-miR-19b | 788 | 5 | 1.02e−009 |
| 1251 | 11 | 152 | 369 | hsa-miR-200a | 953 | 5 | 1.87e−010 |
| 1252 | 11 | 157 | 411 | hsa-miR-200b | 1625 | 5 | 1.23e−010 |
| 1253 | 11 | 156 | 446 | hsa-miR-200c | 3334 | 5 | 9.23e−011 |
| 1254 | 11 | 203 | 504 | hsa-miR-205 | 31815 | 5 | 1.38e−011 |
| 1255 | 11 | 175 | 462 | hsa-miR-21 | 9461 | 5 | 1.3e−009 |
| 1256 | 11 | 115 | 314 | hsa-miR-210 | 694 | 5 | 3.7e−010 |
| 1257 | 11 | 29 | 439 | hsa-miR-214 | 2226 | 5 | 2.98e−011 |
| 1258 | 11 | 18 | 322 | hsa-miR-22 | 5416 | 5 | 7.27e−011 |
| 1259 | 11 | 50 | 364 | hsa-miR-221 | 3091 | 5 | 2.29e−010 |
| 1260 | 11 | 49 | 302 | hsa-miR-222 | 4307 | 5 | 1.53e−010 |
| 1261 | 11 | 67 | 336 | hsa-miR-23a | 2528 | 5 | 2.99e−009 |
| 1262 | 11 | 66 | 306 | hsa-miR-23b | 4519 | 5 | 2.33e−009 |
| 1263 | 11 | 238 | 346, 416 | hsa-miR-24 | 11821 | 5 | 2.37e−009 |
| 1264 | 11 | 90 | 388 | hsa-miR-25 | 597 | 5 | 1.94e−010 |
| 1265 | 11 | 256 | 441, 487 | hsa-miR-26a | 4422 | 5 | 1.28e−009 |
| 1266 | 11 | 259 | 481 | hsa-miR-27a | 4340 | 5 | 1.9e−009 |
| 1267 | 11 | 260 | 280 | hsa-miR-27b | 1453 | 5 | 8.97e−009 |
| 1268 | 11 | 61 | 286 | hsa-miR-296 | 1877 | 5 | 7.88e−010 |
| 1269 | 11 | 167 | 556 | hsa-miR-29a | 1437 | 5 | 7.14e−010 |
| 1270 | 11 | 244 | 287 | hsa-miR-30a-5p | 956 | 5 | 3.07e−012 |
| 1271 | 11 | 243 | 299, 316 | hsa-miR-30c | 644 | 5 | 7.62e−009 |
| 1272 | 11 | 241 | 550 | hsa-miR-30d | 947 | 5 | 7.6e−011 |
| 1273 | 11 | 245 | 431 | hsa-miR-30e-5p | 1348 | 5 | 2.3e−009 |
| 1274 | 11 | 132 | 429 | hsa-miR-31 | 5936 | 5 | 2.22e−009 |
| 1275 | 11 | 1 | 359 | hsa-miR-320 | 5085 | 5 | 1.37e−010 |
| 1276 | 11 | 91 | 368 | hsa-miR-324-3p | 832 | 5 | 3.01e−012 |
| 1277 | 11 | 113 | 538 | hsa-miR-328 | 1276 | 5 | 3.41e−010 |
| 1278 | 11 | 141 | 437 | hsa-miR-33 | 945 | 5 | 3.1e−011 |
| 1279 | 11 | 126 | 380 | hsa-miR-331 | 1071 | 5 | 1.52e−011 |
| 1280 | 11 | 185 | 492 | hsa-miR-335 | 1676 | 5 | 7.96e−010 |
| 1281 | 11 | 194 | 337 | hsa-miR-338 | 2682 | 5 | 4.55e−011 |
| 1282 | 11 | 237 | 480 | hsa-miR-34a | 1860 | 5 | 1.03e−011 |
| 1283 | 11 | 160 | 379, 382 | hsa-miR-365 | 530 | 5 | 4.75e−011 |
| 1284 | 11 | 128 | 294 | hsa-miR-370 | 882 | 5 | 4.16e−010 |
| 1285 | 11 | 35 | 406 | hsa-miR-373* | 551 | 5 | 1.82e−010 |
| 1286 | 11 | 112 | 372 | hsa-miR-422b | 520 | 5 | 8.74e−011 |
| 1287 | 11 | 51 | 497 | hsa-miR-423 | 2829 | 5 | 4.62e−010 |
| 1288 | 11 | 236 | 413 | hsa-miR-449 | 2899 | 5 | 1.25e−009 |
| 1289 | 11 | 44 | 547 | hsa-miR-485-5p | 736 | 5 | 6.16e−010 |
| 1290 | 11 | 63 | 557 | hsa-miR-491 | 2859 | 5 | 2.37e−009 |
| 1291 | 11 | 214 | 376 | hsa-miR-494 | 14139 | 5 | 2.53e−009 |
| 1292 | 11 | 83 | 312 | hsa-miR-497 | 859 | 5 | 2.2e−009 |
| 1293 | 11 | 43 | 418 | hsa-miR-504 | 504 | 5 | 5.58e−011 |
| 1294 | 11 | 258 | 428, 443 | hsa-miR-513 | 617 | 5 | 8.71e−010 |
| 1295 | 11 | 210 | 400 | hsa-miR-518c* | 541 | 5 | 3.71e−010 |
| 1296 | 11 | 103 | 342 | hsa-miR-525 | 583 | 5 | 3.61e−010 |
| 1297 | 11 | 108 | 501 | hsa-miR-526b | 503 | 5 | 2.52e−010 |
| 1298 | 11 | 135 | 315 | hsa-miR-565 | 1451 | 5 | 4.33e−012 |
| 1299 | 11 | 60 | 479 | hsa-miR-638 | 5669 | 5 | 4.49e−010 |
| 1300 | 11 | 195 | 421 | hsa-miR-662 | 580 | 5 | 8.57e−011 |
| 1301 | 11 | 59 | 330 | hsa-miR-663 | 1253 | 5 | 1.49e−010 |
| 1302 | 11 | 186 | 341 | hsa-miR-768-3p | 1546 | 5 | 3.46e−009 |
| 1303 | 11 | 123 | 291 | hsa-miR-801 | 738 | 5 | 1.06e−009 |
| 1304 | 11 | 182 | 304, 317 | hsa-miR-92 | 3726 | 5 | 5.48e−011 |
| 1305 | 11 | 6 | 471 | hsa-miR-93 | 1277 | 5 | 1.81e−011 |
| 1306 | 11 | 14 | 318 | hsa-miR-99a | 5030 | 5 | 4.65e−010 |
| 1307 | 11 | 80 | 509 | hsa-miR-99b | 582 | 5 | 2.33e−010 |
| 1308 | 12 | 196 | 508 | 15_rel | 36396 | 5 | 1.34e−009 |
| 1309 | 12 | 219 | 497 | 27_3 | 511 | 18 | 0 |
| 1310 | 12 | 94 | 394 | 69_6_rel | 6716 | 5 | 1.06e−010 |
| 1311 | 12 | 177 | 366, 464 | 72_12 | 1002 | 5 | 1.23e−011 |
| 1312 | 12 | 97 | 357 | 74_26_rel | 1478 | 5 | 1.63e−009 |
| 1313 | 12 | 223 | 320, 393, 559 | hsa-let-7a | 4494 | 18 | 1.78e−038 |
| 1314 | 12 | 225 | 332 | hsa-let-7b | 6453 | 18 | 0 |
| 1315 | 12 | 224 | 427 | hsa-let-7c | 5496 | 18 | 0 |
| 1316 | 12 | 45 | 277 | hsa-let-7d | 1528 | 18 | 0 |
| 1317 | 12 | 220 | 475 | hsa-let-7e | 1475 | 18 | 0 |
| 1318 | 12 | 226 | 395 | hsa-let-7g | 676 | 18 | 0 |
| 1319 | 12 | 227 | 297 | hsa-let-7i | 819 | 18 | 0 |
| 1320 | 12 | 13 | 397 | hsa-miR-100 | 686 | 18 | 0 |
| 1321 | 12 | 48 | 483, 529 | hsa-miR-103 | 931 | 18 | 0 |
| 1322 | 12 | 2 | 399 | hsa-miR-106a | 907 | 18 | 0 |
| 1323 | 12 | 149 | 392 | hsa-miR-106b | 575 | 18 | 0 |
| 1324 | 12 | 47 | 555 | hsa-miR-107 | 846 | 18 | 0 |
| 1325 | 12 | 199 | 387 | hsa-miR-125a | 2142 | 18 | 0 |
| 1326 | 12 | 198 | 313, 551 | hsa-miR-125b | 5338 | 18 | 0 |
| 1327 | 12 | 204 | 419 | hsa-miR-126 | 1255 | 18 | 0 |
| 1328 | 12 | 86 | 417 | hsa-miR-130a | 1120 | 18 | 0 |
| 1329 | 12 | 266 | 295, 386 | hsa-miR-133a | 783 | 18 | 0 |
| 1330 | 12 | 151 | 472 | hsa-miR-141 | 1778 | 18 | 0 |
| 1331 | 12 | 218 | 527 | hsa-miR-143 | 26609 | 18 | 0 |
| 1332 | 12 | 139 | 281 | hsa-miR-145 | 25278 | 18 | 0 |
| 1333 | 12 | 172 | 329 | hsa-miR-15b | 656 | 18 | 0 |
| 1334 | 12 | 173 | 360, 468 | hsa-miR-16 | 2100 | 18 | 0 |
| 1335 | 12 | 75 | 467 | hsa-miR-17-5p | 673 | 18 | 0 |
| 1336 | 12 | 10 | 371, 461 | hsa-miR-181a | 630 | 18 | 0 |
| 1337 | 12 | 77 | 440 | hsa-miR-191 | 987 | 18 | 0 |
| 1338 | 12 | 109 | 374 | hsa-miR-192 | 2173 | 18 | 0 |
| 1339 | 12 | 17 | 442 | hsa-miR-193a | 1972 | 18 | 0 |
| 1340 | 12 | 246 | 290, 549 | hsa-miR-194 | 3729 | 18 | 0 |
| 1341 | 12 | 170 | 327 | hsa-miR-195 | 888 | 18 | 0 |
| 1342 | 12 | 92 | 410, 432 | hsa-miR-199a | 1608 | 18 | 0 |
| 1343 | 12 | 162 | 410, 432, 473 | hsa-miR-199a* | 832 | 18 | 0 |
| 1344 | 12 | 251 | 282, 495 | hsa-miR-19b | 753 | 18 | 0 |
| 1345 | 12 | 152 | 369 | hsa-miR-200a | 1279 | 18 | 0 |
| 1346 | 12 | 157 | 411 | hsa-miR-200b | 2460 | 18 | 0 |
| 1347 | 12 | 156 | 446 | hsa-miR-200c | 2150 | 18 | 0 |
| 1348 | 12 | 175 | 462 | hsa-miR-21 | 4149 | 18 | 0 |
| 1349 | 12 | 29 | 439 | hsa-miR-214 | 770 | 18 | 0 |
| 1350 | 12 | 18 | 322 | hsa-miR-22 | 1407 | 18 | 0 |
| 1351 | 12 | 50 | 364 | hsa-miR-221 | 724 | 18 | 0 |
| 1352 | 12 | 49 | 302 | hsa-miR-222 | 1178 | 18 | 0 |
| 1353 | 12 | 67 | 336 | hsa-miR-23a | 3126 | 18 | 0 |
| 1354 | 12 | 66 | 306 | hsa-miR-23b | 3304 | 18 | 7.01e−035 |
| 1355 | 12 | 238 | 346, 416 | hsa-miR-24 | 5479 | 18 | 0 |
| 1356 | 12 | 256 | 441, 487 | hsa-miR-26a | 4673 | 18 | 0 |
| 1357 | 12 | 259 | 481 | hsa-miR-27a | 2955 | 18 | 0 |
| 1358 | 12 | 260 | 280 | hsa-miR-27b | 1951 | 18 | 0 |
| 1359 | 12 | 61 | 286 | hsa-miR-296 | 775 | 18 | 0 |
| 1360 | 12 | 167 | 556 | hsa-miR-29a | 1130 | 18 | 9.1e−037 |
| 1361 | 12 | 244 | 287 | hsa-miR-30a-5p | 707 | 18 | 2.39e−038 |
| 1362 | 12 | 242 | 311 | hsa-miR-30b | 602 | 18 | 0 |
| 1363 | 12 | 243 | 299, 316 | hsa-miR-30c | 932 | 18 | 0 |
| 1364 | 12 | 245 | 431 | hsa-miR-30e-5p | 836 | 18 | 0 |
| 1365 | 12 | 1 | 359 | hsa-miR-320 | 1868 | 18 | 0 |
| 1366 | 12 | 237 | 480 | hsa-miR-34a | 528 | 18 | 0 |
| 1367 | 12 | 112 | 372 | hsa-miR-422b | 1369 | 18 | 0 |
| 1368 | 12 | 51 | 497 | hsa-miR-423 | 866 | 18 | 0 |
| 1369 | 12 | 63 | 557 | hsa-miR-491 | 724 | 18 | 0 |
| 1370 | 12 | 214 | 376 | hsa-miR-494 | 7326 | 18 | 0 |
| 1371 | 12 | 83 | 312 | hsa-miR-497 | 1319 | 18 | 0 |
| 1372 | 12 | 135 | 315 | hsa-miR-565 | 1524 | 5 | 2.54e−011 |
| 1373 | 12 | 60 | 479 | hsa-miR-638 | 7992 | 5 | 2.3e−010 |
| 1374 | 12 | 182 | 304, 317 | hsa-miR-92 | 993 | 18 | 0 |
| 1375 | 12 | 6 | 471 | hsa-miR-93 | 652 | 18 | 0 |
| 1376 | 12 | 14 | 318 | hsa-miR-99a | 739 | 18 | 0 |
| 1377 | 12 | 131 | 430 | 6_11 | 662 | 18 | 0 |
| 1378 | 13 | 127 | 361 | 11_1 | 601 | 15 | 9.74e−032 |
| 1379 | 13 | 196 | 508 | 15_rel | 48693 | 7 | 1.24e−015 |
| 1380 | 13 | 219 | 497 | 27_3 | 864 | 15 | 3.72e−033 |
| 1381 | 13 | 145 | 447 | 43_44_rel | 66640 | 5 | 1.51e−011 |
| 1382 | 13 | 94 | 394 | 69_6_rel | 13296 | 7 | 2.15e−015 |
| 1383 | 13 | 177 | 366, 464 | 72_12 | 1133 | 7 | 1.84e−013 |
| 1384 | 13 | 97 | 357 | 74_26_rel | 1465 | 7 | 1.3e−015 |
| 1385 | 13 | 33 | 339, 552 | 96_rel | 54453 | 5 | 5.98e−011 |
| 1386 | 13 | 87 | 301 | gam11 | 5330 | 7 | 5.94e−012 |
| 1387 | 13 | 223 | 320, 393, 559 | hsa-let-7a | 2352 | 15 | 2.92e−030 |
| 1388 | 13 | 225 | 332 | hsa-let-7b | 5804 | 15 | 7.12e−035 |
| 1389 | 13 | 224 | 427 | hsa-let-7c | 3275 | 15 | 3.69e−033 |
| 1390 | 13 | 45 | 277 | hsa-let-7d | 1280 | 15 | 3.42e−033 |
| 1391 | 13 | 220 | 475 | hsa-let-7e | 1487 | 15 | 6.93e−036 |
| 1392 | 13 | 226 | 395 | hsa-let-7g | 823 | 15 | 1.11e−034 |
| 1393 | 13 | 227 | 297 | hsa-let-7i | 1060 | 15 | 3.27e−035 |
| 1394 | 13 | 13 | 397 | hsa-miR-100 | 710 | 15 | 7.21e−034 |
| 1395 | 13 | 48 | 483, 529 | hsa-miR-103 | 874 | 15 | 1.39e−033 |
| 1396 | 13 | 184 | 423 | hsa-miR-105 | 549 | 15 | 5.54e−035 |
| 1397 | 13 | 2 | 399 | hsa-miR-106a | 1058 | 15 | 2.28e−030 |
| 1398 | 13 | 149 | 392 | hsa-miR-106b | 891 | 15 | 5.9e−037 |
| 1399 | 13 | 47 | 555 | hsa-miR-107 | 993 | 15 | 1.27e−034 |
| 1400 | 13 | 199 | 387 | hsa-miR-125a | 1551 | 15 | 1.64e−033 |
| 1401 | 13 | 198 | 313, 551 | hsa-miR-125b | 4721 | 15 | 3.27e−034 |
| 1402 | 13 | 204 | 419 | hsa-miR-126 | 626 | 15 | 9.64e−031 |
| 1403 | 13 | 86 | 417 | hsa-miR-130a | 1273 | 15 | 1.02e−036 |
| 1404 | 13 | 151 | 472 | hsa-miR-141 | 2616 | 15 | 4.46e−035 |
| 1405 | 13 | 218 | 527 | hsa-miR-143 | 6162 | 15 | 7.44e−033 |
| 1406 | 13 | 139 | 281 | hsa-miR-145 | 7483 | 15 | 1.09e−033 |
| 1407 | 13 | 172 | 329 | hsa-miR-15b | 511 | 15 | 1.92e−030 |
| 1408 | 13 | 173 | 360, 468 | hsa-miR-16 | 1132 | 15 | 2.17e−028 |
| 1409 | 13 | 75 | 467 | hsa-miR-17-5p | 674 | 15 | 1.47e−029 |
| 1410 | 13 | 10 | 371, 461 | hsa-miR-181a | 1068 | 15 | 9.54e−036 |
| 1411 | 13 | 77 | 440 | hsa-miR-191 | 1313 | 15 | 2.67e−038 |
| 1412 | 13 | 130 | 440 | hsa-miR-191* | 592 | 7 | 1.13e−013 |
| 1413 | 13 | 109 | 374 | hsa-miR-192 | 1279 | 15 | 9.74e−032 |
| 1414 | 13 | 17 | 442 | hsa-miR-193a | 1501 | 15 | 9.65e−035 |
| 1415 | 13 | 246 | 290, 549 | hsa-miR-194 | 2222 | 15 | 1.39e−031 |
| 1416 | 13 | 92 | 410, 432 | hsa-miR-199a | 1924 | 15 | 1.78e−032 |
| 1417 | 13 | 251 | 282, 495 | hsa-miR-19b | 737 | 15 | 1.67e−032 |
| 1418 | 13 | 152 | 369 | hsa-miR-200a | 1299 | 15 | 7.66e−033 |
| 1419 | 13 | 157 | 411 | hsa-miR-200b | 2703 | 15 | 1.45e−032 |
| 1420 | 13 | 156 | 446 | hsa-miR-200c | 3677 | 15 | 2.28e−035 |
| 1421 | 13 | 175 | 462 | hsa-miR-21 | 5514 | 15 | 3.59e−030 |
| 1422 | 13 | 29 | 439 | hsa-miR-214 | 1195 | 15 | 3.12e−034 |
| 1423 | 13 | 18 | 322 | hsa-miR-22 | 1466 | 15 | 3.26e−037 |
| 1424 | 13 | 50 | 364 | hsa-miR-221 | 1068 | 15 | 0 |
| 1425 | 13 | 49 | 302 | hsa-miR-222 | 1793 | 15 | 4.85e−038 |
| 1426 | 13 | 67 | 336 | hsa-miR-23a | 1947 | 15 | 2.84e−029 |
| 1427 | 13 | 66 | 306 | hsa-miR-23b | 2514 | 15 | 2.7e−028 |
| 1428 | 13 | 238 | 346, 416 | hsa-miR-24 | 4619 | 15 | 9.49e−037 |
| 1429 | 13 | 90 | 388 | hsa-miR-25 | 515 | 15 | 1.45e−032 |
| 1430 | 13 | 256 | 441, 487 | hsa-miR-26a | 2291 | 15 | 3.68e−032 |
| 1431 | 13 | 259 | 481 | hsa-miR-27a | 2188 | 15 | 4.09e−033 |
| 1432 | 13 | 260 | 280 | hsa-miR-27b | 681 | 15 | 6.25e−028 |
| 1433 | 13 | 61 | 286 | hsa-miR-296 | 863 | 15 | 1.46e−031 |
| 1434 | 13 | 167 | 556 | hsa-miR-29a | 1010 | 15 | 4.97e−027 |
| 1435 | 13 | 244 | 287 | hsa-miR-30a-5p | 634 | 15 | 7.22e−032 |
| 1436 | 13 | 241 | 550 | hsa-miR-30d | 546 | 15 | 1.24e−029 |
| 1437 | 13 | 245 | 431 | hsa-miR-30e-5p | 517 | 15 | 3.35e−029 |
| 1438 | 13 | 1 | 359 | hsa-miR-320 | 2676 | 15 | 4.22e−031 |
| 1439 | 13 | 237 | 480 | hsa-miR-34a | 901 | 15 | 3.38e−038 |
| 1440 | 13 | 112 | 372 | hsa-miR-422b | 798 | 15 | 4.31e−035 |
| 1441 | 13 | 51 | 497 | hsa-miR-423 | 1229 | 15 | 1.94e−032 |
| 1442 | 13 | 236 | 413 | hsa-miR-449 | 953 | 15 | 3.35e−030 |
| 1443 | 13 | 63 | 557 | hsa-miR-491 | 2910 | 15 | 1.09e−029 |
| 1444 | 13 | 214 | 376 | hsa-miR-494 | 7950 | 15 | 8.3e−033 |
| 1445 | 13 | 83 | 312 | hsa-miR-497 | 587 | 15 | 1.34e−031 |
| 1446 | 13 | 135 | 315 | hsa-miR-565 | 649 | 7 | 2.71e−015 |
| 1447 | 13 | 60 | 479 | hsa-miR-638 | 14944 | 7 | 1.4e−014 |
| 1448 | 13 | 59 | 330 | hsa-miR-663 | 1013 | 7 | 5.92e−012 |
| 1449 | 13 | 36 | 385 | hsa-miR-766 | 580 | 5 | 1.34e−009 |
| 1450 | 13 | 186 | 341 | hsa-miR-768-3p | 541 | 5 | 3.91e−009 |
| 1451 | 13 | 123 | 291 | hsa-miR-801 | 652 | 5 | 8.47e−010 |
| 1452 | 13 | 182 | 304, 317 | hsa-miR-92 | 2408 | 15 | 9.88e−032 |
| 1453 | 13 | 6 | 471 | hsa-miR-93 | 904 | 15 | 5.76e−036 |
| 1454 | 13 | 14 | 318 | hsa-miR-99a | 797 | 15 | 4.27e−034 |
| 1455 | 14 | 127 | 361 | 11_1 | 1090 | 8 | 1.49e−018 |
| 1456 | 14 | 219 | 497 | 27_3 | 655 | 8 | 3.07e−017 |
| 1457 | 14 | 223 | 320, 393, 559 | hsa-let-7a | 5610 | 8 | 1.5e−015 |
| 1458 | 14 | 225 | 332 | hsa-let-7b | 6390 | 8 | 1.35e−017 |
| 1459 | 14 | 224 | 427 | hsa-let-7c | 6116 | 8 | 1.17e−017 |
| 1460 | 14 | 45 | 277 | hsa-let-7d | 2116 | 8 | 6.65e−017 |
| 1461 | 14 | 220 | 475 | hsa-let-7e | 1525 | 8 | 3e−019 |
| 1462 | 14 | 222 | 482, 558 | hsa-let-7f | 629 | 8 | 1.4e−016 |
| 1463 | 14 | 226 | 395 | hsa-let-7g | 939 | 8 | 3.82e−018 |
| 1464 | 14 | 227 | 297 | hsa-let-7i | 1557 | 8 | 4.88e−018 |
| 1465 | 14 | 13 | 397 | hsa-miR-100 | 577 | 8 | 2.79e−017 |
| 1466 | 14 | 48 | 483, 529 | hsa-miR-103 | 1199 | 8 | 8.15e−018 |
| 1467 | 14 | 184 | 423 | hsa-miR-105 | 797 | 8 | 1.94e−019 |
| 1468 | 14 | 2 | 399 | hsa-miR-106a | 2041 | 8 | 6.35e−017 |
| 1469 | 14 | 149 | 392 | hsa-miR-106b | 1081 | 8 | 2.04e−019 |
| 1470 | 14 | 47 | 555 | hsa-miR-107 | 1247 | 8 | 7.91e−018 |
| 1471 | 14 | 199 | 387 | hsa-miR-125a | 1801 | 8 | 8.5e−017 |
| 1472 | 14 | 198 | 313, 551 | hsa-miR-125b | 5795 | 8 | 4.38e−018 |
| 1473 | 14 | 204 | 419 | hsa-miR-126 | 1005 | 8 | 1.92e−016 |
| 1474 | 14 | 86 | 417 | hsa-miR-130a | 1421 | 8 | 6.09e−018 |
| 1475 | 14 | 151 | 472 | hsa-miR-141 | 3075 | 8 | 6.47e−017 |
| 1476 | 14 | 218 | 527 | hsa-miR-143 | 8030 | 8 | 2.42e−016 |
| 1477 | 14 | 139 | 281 | hsa-miR-145 | 9469 | 8 | 2.63e−016 |
| 1478 | 14 | 190 | 458 | hsa-miR-148a | 594 | 8 | 1.44e−016 |
| 1479 | 14 | 172 | 329 | hsa-miR-15b | 1114 | 8 | 8.29e−017 |
| 1480 | 14 | 173 | 360, 468 | hsa-miR-16 | 3242 | 8 | 1.55e−016 |
| 1481 | 14 | 75 | 467 | hsa-miR-17-5p | 1567 | 8 | 1.06e−016 |
| 1482 | 14 | 10 | 371, 461 | hsa-miR-181a | 1113 | 8 | 2.12e−017 |
| 1483 | 14 | 77 | 440 | hsa-miR-191 | 1386 | 8 | 6.23e−020 |
| 1484 | 14 | 109 | 374 | hsa-miR-192 | 2390 | 8 | 3.14e−016 |
| 1485 | 14 | 17 | 442 | hsa-miR-193a | 2051 | 8 | 1.12e−017 |
| 1486 | 14 | 246 | 290, 549 | hsa-miR-194 | 3645 | 8 | 5.01e−017 |
| 1487 | 14 | 170 | 327 | hsa-miR-195 | 647 | 8 | 5.64e−016 |
| 1488 | 14 | 92 | 410, 432 | hsa-miR-199a | 2180 | 8 | 1.23e−016 |
| 1489 | 14 | 162 | 410, 432, 473 | hsa-miR-199a* | 1282 | 8 | 3.27e−015 |
| 1490 | 14 | 251 | 282, 495 | hsa-miR-19b | 1318 | 8 | 5.93e−018 |
| 1491 | 14 | 152 | 369 | hsa-miR-200a | 2095 | 8 | 3.99e−017 |
| 1492 | 14 | 157 | 411 | hsa-miR-200b | 5065 | 8 | 5.17e−019 |
| 1493 | 14 | 156 | 446 | hsa-miR-200c | 4866 | 8 | 6.82e−018 |
| 1494 | 14 | 150 | 351 | hsa-miR-20a | 579 | 8 | 3.7e−017 |
| 1495 | 14 | 175 | 462 | hsa-miR-21 | 14957 | 8 | 3.24e−016 |
| 1496 | 14 | 29 | 439 | hsa-miR-214 | 1062 | 8 | 2.78e−018 |
| 1497 | 14 | 18 | 322 | hsa-miR-22 | 1439 | 8 | 6.05e−020 |
| 1498 | 14 | 50 | 364 | hsa-miR-221 | 1169 | 8 | 1.21e−018 |
| 1499 | 14 | 49 | 302 | hsa-miR-222 | 1863 | 8 | 4.52e−019 |
| 1500 | 14 | 67 | 336 | hsa-miR-23a | 4370 | 8 | 8.92e−016 |
| 1501 | 14 | 66 | 306 | hsa-miR-23b | 6094 | 8 | 1.19e−015 |
| 1502 | 14 | 238 | 346, 416 | hsa-miR-24 | 6311 | 8 | 5.24e−019 |
| 1503 | 14 | 90 | 388 | hsa-miR-25 | 758 | 8 | 1.1e−019 |
| 1504 | 14 | 256 | 441, 487 | hsa-miR-26a | 4228 | 8 | 2.79e−017 |
| 1505 | 14 | 259 | 481 | hsa-miR-27a | 3325 | 8 | 4.37e−016 |
| 1506 | 14 | 260 | 280 | hsa-miR-27b | 1821 | 8 | 1.04e−015 |
| 1507 | 14 | 61 | 286 | hsa-miR-296 | 659 | 8 | 9.11e−017 |
| 1508 | 14 | 167 | 556 | hsa-miR-29a | 4269 | 8 | 7.95e−017 |
| 1509 | 14 | 168 | 303, 530 | hsa-miR-29b | 959 | 8 | 5.43e−015 |
| 1510 | 14 | 244 | 287 | hsa-miR-30a-5p | 751 | 8 | 2.11e−017 |
| 1511 | 14 | 242 | 311 | hsa-miR-30b | 586 | 8 | 6.71e−016 |
| 1512 | 14 | 243 | 299, 316 | hsa-miR-30c | 813 | 8 | 3.97e−016 |
| 1513 | 14 | 241 | 550 | hsa-miR-30d | 1191 | 8 | 4.01e−018 |
| 1514 | 14 | 245 | 431 | hsa-miR-30e-5p | 928 | 8 | 7.55e−018 |
| 1515 | 14 | 1 | 359 | hsa-miR-320 | 1609 | 8 | 8e−018 |
| 1516 | 14 | 237 | 480 | hsa-miR-34a | 808 | 8 | 1.91e−019 |
| 1517 | 14 | 112 | 372 | hsa-miR-422b | 727 | 8 | 8.22e−018 |
| 1518 | 14 | 51 | 497 | hsa-miR-423 | 954 | 8 | 4.92e−016 |
| 1519 | 14 | 158 | 378 | hsa-miR-429 | 534 | 8 | 6.44e−018 |
| 1520 | 14 | 236 | 413 | hsa-miR-449 | 670 | 8 | 1.57e−014 |
| 1521 | 14 | 63 | 557 | hsa-miR-491 | 2365 | 8 | 5.32e−015 |
| 1522 | 14 | 214 | 376 | hsa-miR-494 | 12615 | 8 | 1.75e−016 |
| 1523 | 14 | 83 | 312 | hsa-miR-497 | 751 | 8 | 5.51e−016 |
| 1524 | 14 | 182 | 304, 317 | hsa-miR-92 | 2389 | 8 | 4.86e−016 |
| 1525 | 14 | 6 | 471 | hsa-miR-93 | 1072 | 8 | 1.04e−017 |
| 1526 | 14 | 14 | 318 | hsa-miR-99a | 623 | 8 | 4.25e−018 |
| 1527 | 14 | 131 | 430 | 6_11 | 856 | 8 | 3.13e−015 |
| 1528 | 15 | 223 | 320, 393, 559 | hsa-let-7a | 786 | 5 | 1.47e−009 |
| 1529 | 15 | 225 | 332 | hsa-let-7b | 2252 | 5 | 1.17e−009 |
| 1530 | 15 | 224 | 427 | hsa-let-7c | 1683 | 5 | 1.58e−009 |
| 1531 | 15 | 45 | 277 | hsa-let-7d | 642 | 5 | 6.24e−011 |
| 1532 | 15 | 220 | 475 | hsa-let-7e | 576 | 5 | 1.4e−011 |
| 1533 | 15 | 227 | 297 | hsa-let-7i | 662 | 5 | 1.83e−011 |
| 1534 | 15 | 199 | 387 | hsa-miR-125a | 838 | 5 | 2.98e−010 |
| 1535 | 15 | 198 | 313, 551 | hsa-miR-125b | 3011 | 5 | 4.3e−012 |
| 1536 | 15 | 86 | 417 | hsa-miR-130a | 1043 | 5 | 9.58e−009 |
| 1537 | 15 | 151 | 472 | hsa-miR-141 | 1883 | 5 | 1.54e−009 |
| 1538 | 15 | 218 | 527 | hsa-miR-143 | 2574 | 5 | 3.5e−009 |
| 1539 | 15 | 139 | 281 | hsa-miR-145 | 2947 | 5 | 5.73e−010 |
| 1540 | 15 | 77 | 440 | hsa-miR-191 | 532 | 5 | 2.66e−010 |
| 1541 | 15 | 246 | 290, 549 | hsa-miR-194 | 1098 | 5 | 3.1e−009 |
| 1542 | 15 | 92 | 410, 432 | hsa-miR-199a | 553 | 5 | 3.43e−010 |
| 1543 | 15 | 152 | 369 | hsa-miR-200a | 895 | 5 | 1.26e−009 |
| 1544 | 15 | 157 | 411 | hsa-miR-200b | 1835 | 5 | 1.57e−009 |
| 1545 | 15 | 156 | 446 | hsa-miR-200c | 2539 | 5 | 5.72e−010 |
| 1546 | 15 | 175 | 462 | hsa-miR-21 | 3326 | 5 | 2.18e−009 |
| 1547 | 15 | 18 | 322 | hsa-miR-22 | 1162 | 5 | 1.07e−010 |
| 1548 | 15 | 49 | 302 | hsa-miR-222 | 1118 | 5 | 8.57e−012 |
| 1549 | 15 | 67 | 336 | hsa-miR-23a | 573 | 5 | 1.02e−008 |
| 1550 | 15 | 66 | 306 | hsa-miR-23b | 845 | 5 | 2.02e−008 |
| 1551 | 15 | 238 | 346, 416 | hsa-miR-24 | 1575 | 5 | 3.05e−010 |
| 1552 | 15 | 256 | 441, 487 | hsa-miR-26a | 1020 | 5 | 1.75e−010 |
| 1553 | 15 | 259 | 481 | hsa-miR-27a | 720 | 5 | 1.64e−009 |
| 1554 | 15 | 167 | 556 | hsa-miR-29a | 529 | 5 | 1.12e−008 |
| 1555 | 15 | 1 | 359 | hsa-miR-320 | 1419 | 5 | 5.34e−010 |
| 1556 | 15 | 51 | 497 | hsa-miR-423 | 814 | 5 | 5.99e−009 |
| 1557 | 15 | 236 | 413 | hsa-miR-449 | 849 | 5 | 2.39e−008 |
| 1558 | 15 | 63 | 557 | hsa-miR-491 | 1108 | 5 | 2.69e−009 |
| 1559 | 15 | 214 | 376 | hsa-miR-494 | 4426 | 5 | 2.01e−010 |
| 1560 | 15 | 182 | 304, 317 | hsa-miR-92 | 1188 | 5 | 2.37e−009 |
| 1561 | 15 | 6 | 471 | hsa-miR-93 | 548 | 5 | 1.62e−010 |
| 1562 | 16 | 196 | 508 | 15_rel | 31942 | 7 | 2.01e−017 |
| 1563 | 16 | 219 | 497 | 27_3 | 642 | 7 | 8.16e−015 |
| 1564 | 16 | 145 | 447 | 43_44_rel | 33723 | 7 | 5.76e−017 |
| 1565 | 16 | 94 | 394 | 69_6_rel | 2183 | 7 | 3.95e−015 |
| 1566 | 16 | 97 | 357 | 74_26_rel | 990 | 7 | 3.17e−017 |
| 1567 | 16 | 33 | 339, 552 | 96_rel | 22208 | 7 | 2.17e−016 |
| 1568 | 16 | 87 | 301 | gam11 | 4956 | 7 | 3.62e−015 |
| 1569 | 16 | 223 | 320, 393, 559 | hsa-let-7a | 805 | 7 | 1.35e−014 |
| 1570 | 16 | 225 | 332 | hsa-let-7b | 5218 | 7 | 1.11e−015 |
| 1571 | 16 | 224 | 427 | hsa-let-7c | 1059 | 7 | 1.26e−014 |
| 1572 | 16 | 45 | 277 | hsa-let-7d | 833 | 7 | 2.93e−016 |
| 1573 | 16 | 220 | 475 | hsa-let-7e | 681 | 7 | 7.33e−017 |
| 1574 | 16 | 48 | 483, 529 | hsa-miR-103 | 1250 | 7 | 1.25e−014 |
| 1575 | 16 | 2 | 399 | hsa-miR-106a | 1204 | 7 | 4.77e−014 |
| 1576 | 16 | 149 | 392 | hsa-miR-106b | 655 | 7 | 3.08e−017 |
| 1577 | 16 | 47 | 555 | hsa-miR-107 | 1126 | 7 | 2.48e−015 |
| 1578 | 16 | 198 | 313, 551 | hsa-miR-125b | 763 | 7 | 1.09e−014 |
| 1579 | 16 | 86 | 417 | hsa-miR-130a | 599 | 7 | 1.6e−015 |
| 1580 | 16 | 151 | 472 | hsa-miR-141 | 1710 | 7 | 1.55e−014 |
| 1581 | 16 | 218 | 527 | hsa-miR-143 | 1146 | 7 | 2.91e−014 |
| 1582 | 16 | 139 | 281 | hsa-miR-145 | 1627 | 7 | 1.21e−014 |
| 1583 | 16 | 173 | 360, 468 | hsa-miR-16 | 856 | 7 | 4.08e−014 |
| 1584 | 16 | 75 | 467 | hsa-miR-17-5p | 566 | 7 | 4.89e−014 |
| 1585 | 16 | 10 | 371, 461 | hsa-miR-181a | 820 | 7 | 6.93e−015 |
| 1586 | 16 | 77 | 440 | hsa-miR-191 | 769 | 7 | 4.58e−015 |
| 1587 | 16 | 17 | 442 | hsa-miR-193a | 580 | 7 | 8.65e−016 |
| 1588 | 16 | 92 | 410, 432 | hsa-miR-199a | 709 | 7 | 4.34e−015 |
| 1589 | 16 | 251 | 282, 495 | hsa-miR-19b | 526 | 7 | 1.8e−015 |
| 1590 | 16 | 157 | 411 | hsa-miR-200b | 620 | 7 | 1.21e−014 |
| 1591 | 16 | 156 | 446 | hsa-miR-200c | 4192 | 7 | 1.39e−014 |
| 1592 | 16 | 203 | 504 | hsa-miR-205 | 2488 | 7 | 5.42e−012 |
| 1593 | 16 | 175 | 462 | hsa-miR-21 | 1426 | 7 | 1.81e−013 |
| 1594 | 16 | 29 | 439 | hsa-miR-214 | 581 | 7 | 4.45e−016 |
| 1595 | 16 | 18 | 322 | hsa-miR-22 | 751 | 7 | 2.38e−014 |
| 1596 | 16 | 50 | 364 | hsa-miR-221 | 889 | 7 | 2.2e−014 |
| 1597 | 16 | 49 | 302 | hsa-miR-222 | 1050 | 7 | 5.33e−015 |
| 1598 | 16 | 67 | 336 | hsa-miR-23a | 1194 | 7 | 2.99e−014 |
| 1599 | 16 | 66 | 306 | hsa-miR-23b | 2371 | 7 | 3.63e−015 |
| 1600 | 16 | 238 | 346, 416 | hsa-miR-24 | 4862 | 7 | 3.69e−015 |
| 1601 | 16 | 256 | 441, 487 | hsa-miR-26a | 965 | 7 | 6.1e−015 |
| 1602 | 16 | 259 | 481 | hsa-miR-27a | 1497 | 7 | 2.49e−014 |
| 1603 | 16 | 61 | 286 | hsa-miR-296 | 695 | 7 | 2.32e−014 |
| 1604 | 16 | 241 | 550 | hsa-miR-30d | 546 | 7 | 3.37e−015 |
| 1605 | 16 | 132 | 429 | hsa-miR-31 | 2037 | 7 | 1.34e−013 |
| 1606 | 16 | 1 | 359 | hsa-miR-320 | 1685 | 7 | 3.94e−015 |
| 1607 | 16 | 185 | 492 | hsa-miR-335 | 700 | 7 | 1.78e−014 |
| 1608 | 16 | 194 | 337 | hsa-miR-338 | 888 | 7 | 2.06e−016 |
| 1609 | 16 | 51 | 497 | hsa-miR-423 | 1060 | 7 | 1.8e−015 |
| 1610 | 16 | 236 | 413 | hsa-miR-449 | 587 | 7 | 1.15e−015 |
| 1611 | 16 | 63 | 557 | hsa-miR-491 | 938 | 7 | 5.59e−015 |
| 1612 | 16 | 214 | 376 | hsa-miR-494 | 3330 | 7 | 4.51e−015 |
| 1613 | 16 | 135 | 315 | hsa-miR-565 | 738 | 7 | 7.54e−014 |
| 1614 | 16 | 60 | 479 | hsa-miR-638 | 1477 | 7 | 3.95e−016 |
| 1615 | 16 | 59 | 330 | hsa-miR-663 | 840 | 7 | 6.1e−016 |
| 1616 | 16 | 182 | 304, 317 | hsa-miR-92 | 1294 | 7 | 1.51e−013 |
| 1617 | 16 | 6 | 471 | hsa-miR-93 | 1425 | 7 | 5.56e−014 |
| 1618 | 17 | 127 | 361 | 11_1 | 596 | 6 | 8.54e−013 |
| 1619 | 17 | 196 | 508 | 15_rel | 46272 | 6 | 6.65e−016 |
| 1620 | 17 | 219 | 497 | 27_3 | 981 | 6 | 4.17e−014 |
| 1621 | 17 | 145 | 447 | 43_44_rel | 44790 | 6 | 1.66e−016 |
| 1622 | 17 | 94 | 394 | 69_6_rel | 5371 | 6 | 9.32e−014 |
| 1623 | 17 | 177 | 366, 464 | 72_12 | 1666 | 6 | 1.16e−012 |
| 1624 | 17 | 33 | 339, 552 | 96_rel | 25932 | 6 | 1e−015 |
| 1625 | 17 | 87 | 301 | gam11 | 11767 | 6 | 2.2e−013 |
| 1626 | 17 | 223 | 320, 393, 559 | hsa-let-7a | 3692 | 6 | 2e−013 |
| 1627 | 17 | 223 | 320, 393, 559 | hsa-let-7a | 1489 | 6 | 1.68e−012 |
| 1628 | 17 | 225 | 332 | hsa-let-7b | 16797 | 6 | 1.15e−012 |
| 1629 | 17 | 224 | 427 | hsa-let-7c | 7756 | 6 | 6.41e−014 |
| 1630 | 17 | 45 | 277 | hsa-let-7d | 1588 | 6 | 1.43e−013 |
| 1631 | 17 | 220 | 475 | hsa-let-7e | 1855 | 6 | 1.81e−014 |
| 1632 | 17 | 226 | 395 | hsa-let-7g | 696 | 6 | 3.73e−015 |
| 1633 | 17 | 227 | 297 | hsa-let-7i | 1126 | 6 | 2.42e−013 |
| 1634 | 17 | 13 | 397 | hsa-miR-100 | 1314 | 6 | 2.18e−012 |
| 1635 | 17 | 48 | 483, 529 | hsa-miR-103 | 2290 | 6 | 1.67e−012 |
| 1636 | 17 | 2 | 399 | hsa-miR-106a | 972 | 6 | 1.66e−013 |
| 1637 | 17 | 149 | 392 | hsa-miR-106b | 975 | 6 | 2.54e−015 |
| 1638 | 17 | 47 | 555 | hsa-miR-107 | 1904 | 6 | 5.8e−013 |
| 1639 | 17 | 199 | 387 | hsa-miR-125a | 2424 | 6 | 3.06e−014 |
| 1640 | 17 | 198 | 313, 551 | hsa-miR-125b | 7601 | 6 | 1.27e−012 |
| 1641 | 17 | 204 | 419 | hsa-miR-126 | 883 | 6 | 2.92e−012 |
| 1642 | 17 | 86 | 417 | hsa-miR-130a | 1116 | 6 | 8.51e−014 |
| 1643 | 17 | 151 | 472 | hsa-miR-141 | 3011 | 6 | 3.92e−012 |
| 1644 | 17 | 218 | 527 | hsa-miR-143 | 7789 | 6 | 1.38e−012 |
| 1645 | 17 | 139 | 281 | hsa-miR-145 | 5685 | 6 | 1.52e−012 |
| 1646 | 17 | 209 | 507 | hsa-miR-150 | 921 | 6 | 1.59e−013 |
| 1647 | 17 | 173 | 360, 468 | hsa-miR-16 | 3280 | 6 | 8.71e−014 |
| 1648 | 17 | 75 | 467 | hsa-miR-17-5p | 627 | 6 | 5.69e−015 |
| 1649 | 17 | 10 | 371, 461 | hsa-miR-181a | 1204 | 6 | 1.44e−012 |
| 1650 | 17 | 77 | 440 | hsa-miR-191 | 1731 | 6 | 1.4e−013 |
| 1651 | 17 | 109 | 374 | hsa-miR-192 | 649 | 6 | 2.5e−011 |
| 1652 | 17 | 17 | 442 | hsa-miR-193a | 1415 | 6 | 2e−013 |
| 1653 | 17 | 246 | 290, 549 | hsa-miR-194 | 1364 | 6 | 1.26e−010 |
| 1654 | 17 | 92 | 410, 432 | hsa-miR-199a | 5007 | 6 | 2.26e−012 |
| 1655 | 17 | 251 | 282, 495 | hsa-miR-19b | 696 | 6 | 2.13e−013 |
| 1656 | 17 | 152 | 369 | hsa-miR-200a | 1622 | 6 | 1.72e−012 |
| 1657 | 17 | 157 | 411 | hsa-miR-200b | 2136 | 6 | 4.05e−012 |
| 1658 | 17 | 156 | 446 | hsa-miR-200c | 4018 | 6 | 7.78e−013 |
| 1659 | 17 | 175 | 462 | hsa-miR-21 | 10551 | 6 | 3.1e−012 |
| 1660 | 17 | 115 | 314 | hsa-miR-210 | 595 | 6 | 3.98e−014 |
| 1661 | 17 | 29 | 439 | hsa-miR-214 | 1904 | 6 | 1.46e−012 |
| 1662 | 17 | 18 | 322 | hsa-miR-22 | 3181 | 6 | 7.99e−014 |
| 1663 | 17 | 50 | 364 | hsa-miR-221 | 2976 | 6 | 1.68e−013 |
| 1664 | 17 | 49 | 302 | hsa-miR-222 | 4073 | 6 | 2.14e−013 |
| 1665 | 17 | 67 | 336 | hsa-miR-23a | 4616 | 6 | 6.82e−014 |
| 1666 | 17 | 66 | 306 | hsa-miR-23b | 1301 | 6 | 1.01e−010 |
| 1667 | 17 | 238 | 346, 416 | hsa-miR-24 | 10451 | 6 | 2.25e−013 |
| 1668 | 17 | 90 | 388 | hsa-miR-25 | 534 | 6 | 3.56e−014 |
| 1669 | 17 | 256 | 441, 487 | hsa-miR-26a | 5569 | 6 | 1.07e−013 |
| 1670 | 17 | 259 | 481 | hsa-miR-27a | 4652 | 6 | 2.32e−013 |
| 1671 | 17 | 260 | 280 | hsa-miR-27b | 1221 | 6 | 3.03e−013 |
| 1672 | 17 | 61 | 286 | hsa-miR-296 | 1598 | 6 | 2.99e−014 |
| 1673 | 17 | 167 | 556 | hsa-miR-29a | 2127 | 6 | 1.82e−013 |
| 1674 | 17 | 244 | 287 | hsa-miR-30a-5p | 1816 | 6 | 3.75e−013 |
| 1675 | 17 | 241 | 550 | hsa-miR-30d | 548 | 6 | 1.58e−012 |
| 1676 | 17 | 132 | 429 | hsa-miR-31 | 759 | 6 | 3.49e−012 |
| 1677 | 17 | 1 | 359 | hsa-miR-320 | 5992 | 6 | 2.99e−015 |
| 1678 | 17 | 113 | 538 | hsa-miR-328 | 504 | 6 | 3.6e−014 |
| 1679 | 17 | 141 | 437 | hsa-miR-33 | 658 | 6 | 3.57e−015 |
| 1680 | 17 | 126 | 380 | hsa-miR-331 | 965 | 6 | 1.09e−014 |
| 1681 | 17 | 194 | 337 | hsa-miR-338 | 3100 | 6 | 1.63e−014 |
| 1682 | 17 | 237 | 480 | hsa-miR-34a | 1467 | 6 | 1.81e−014 |
| 1683 | 17 | 51 | 497 | hsa-miR-423 | 1609 | 6 | 9.11e−016 |
| 1684 | 17 | 236 | 413 | hsa-miR-449 | 1078 | 6 | 2.15e−013 |
| 1685 | 17 | 63 | 557 | hsa-miR-491 | 1605 | 6 | 1.17e−012 |
| 1686 | 17 | 214 | 376 | hsa-miR-494 | 6576 | 6 | 9.77e−013 |
| 1687 | 17 | 83 | 312 | hsa-miR-497 | 574 | 6 | 1.44e−012 |
| 1688 | 17 | 135 | 315 | hsa-miR-565 | 869 | 6 | 1.62e−013 |
| 1689 | 17 | 60 | 479 | hsa-miR-638 | 4482 | 6 | 2.93e−013 |
| 1690 | 17 | 59 | 330 | hsa-miR-663 | 1705 | 6 | 3.36e−014 |
| 1691 | 17 | 186 | 341 | hsa-miR-768-3p | 2511 | 6 | 8.4e−015 |
| 1692 | 17 | 123 | 291 | hsa-miR-801 | 1115 | 6 | 5.06e−014 |
| 1693 | 17 | 182 | 304, 317 | hsa-miR-92 | 2371 | 6 | 2.87e−014 |
| 1694 | 17 | 6 | 471 | hsa-miR-93 | 1381 | 6 | 1.46e−013 |
| 1695 | 17 | 14 | 318 | hsa-miR-99a | 1170 | 6 | 1.34e−011 |
| 1696 | 18 | 127 | 361 | 11_1 | 573 | 6 | 7.07e−011 |
| 1697 | 18 | 196 | 508 | 15_rel | 27708 | 6 | 5.46e−008 |
| 1698 | 18 | 219 | 497 | 27_3 | 528 | 6 | 2.41e−011 |
| 1699 | 18 | 94 | 394 | 69_6_rel | 1240 | 6 | 2.58e−009 |
| 1700 | 18 | 177 | 366, 464 | 72_12 | 3017 | 6 | 5.89e−009 |
| 1701 | 18 | 97 | 357 | 74_26_rel | 730 | 6 | 2.69e−009 |
| 1702 | 18 | 87 | 301 | gam11 | 5869 | 6 | 3.13e−011 |
| 1703 | 18 | 125 | 532 | 70_16 | 809 | 6 | 4.35e−011 |
| 1704 | 18 | 163 | 402 | 43_7 | 607 | 6 | 5.95e−012 |
| 1705 | 18 | 223 | 320, 393, 559 | hsa-let-7a | 3183 | 6 | 1.06e−010 |
| 1706 | 18 | 225 | 332 | hsa-let-7b | 24481 | 6 | 6.81e−012 |
| 1707 | 18 | 224 | 427 | hsa-let-7c | 6769 | 6 | 5.87e−012 |
| 1708 | 18 | 45 | 277 | hsa-let-7d | 1851 | 6 | 4.95e−011 |
| 1709 | 18 | 220 | 475 | hsa-let-7e | 1958 | 6 | 8.9e−012 |
| 1710 | 18 | 227 | 297 | hsa-let-7i | 571 | 6 | 2.75e−011 |
| 1711 | 18 | 13 | 397 | hsa-miR-100 | 1461 | 6 | 1.95e−011 |
| 1712 | 18 | 48 | 483, 529 | hsa-miR-103 | 2897 | 6 | 2.17e−010 |
| 1713 | 18 | 184 | 423 | hsa-miR-105 | 657 | 6 | 6.08e−012 |
| 1714 | 18 | 2 | 399 | hsa-miR-106a | 832 | 6 | 1.03e−011 |
| 1715 | 18 | 47 | 555 | hsa-miR-107 | 2359 | 6 | 1.2e−010 |
| 1716 | 18 | 165 | 340 | hsa-miR-10b | 523 | 6 | 7.23e−011 |
| 1717 | 18 | 199 | 387 | hsa-miR-125a | 3487 | 6 | 6.23e−012 |
| 1718 | 18 | 198 | 313, 551 | hsa-miR-125b | 8300 | 6 | 1.08e−011 |
| 1719 | 18 | 204 | 419 | hsa-miR-126 | 1624 | 6 | 5.7e−010 |
| 1720 | 18 | 86 | 417 | hsa-miR-130a | 816 | 6 | 4.97e−011 |
| 1721 | 18 | 151 | 472 | hsa-miR-141 | 618 | 6 | 7.19e−010 |
| 1722 | 18 | 218 | 527 | hsa-miR-143 | 4580 | 6 | 4.58e−010 |
| 1723 | 18 | 139 | 281 | hsa-miR-145 | 5835 | 6 | 9.14e−011 |
| 1724 | 18 | 212 | 333 | hsa-miR-149 | 596 | 6 | 9.32e−011 |
| 1725 | 18 | 173 | 360, 468 | hsa-miR-16 | 1135 | 6 | 3.77e−010 |
| 1726 | 18 | 10 | 371, 461 | hsa-miR-181a | 1489 | 6 | 8.51e−013 |
| 1727 | 18 | 77 | 440 | hsa-miR-191 | 1268 | 6 | 7.57e−012 |
| 1728 | 18 | 17 | 442 | hsa-miR-193a | 1354 | 6 | 9.86e−011 |
| 1729 | 18 | 246 | 290, 549 | hsa-miR-194 | 903 | 6 | 3.08e−008 |
| 1730 | 18 | 92 | 410, 432 | hsa-miR-199a | 2070 | 6 | 1.5e−010 |
| 1731 | 18 | 152 | 369 | hsa-miR-200a | 865 | 6 | 4.87e−012 |
| 1732 | 18 | 157 | 411 | hsa-miR-200b | 1101 | 6 | 1.48e−011 |
| 1733 | 18 | 156 | 446 | hsa-miR-200c | 1433 | 6 | 4.24e−011 |
| 1734 | 18 | 175 | 462 | hsa-miR-21 | 1815 | 6 | 7.53e−009 |
| 1735 | 18 | 29 | 439 | hsa-miR-214 | 716 | 6 | 1.92e−011 |
| 1736 | 18 | 18 | 322 | hsa-miR-22 | 3279 | 6 | 8.3e−010 |
| 1737 | 18 | 50 | 364 | hsa-miR-221 | 1411 | 6 | 2.4e−013 |
| 1738 | 18 | 49 | 302 | hsa-miR-222 | 1825 | 6 | 3.65e−011 |
| 1739 | 18 | 67 | 336 | hsa-miR-23a | 1687 | 6 | 8.43e−010 |
| 1740 | 18 | 66 | 306 | hsa-miR-23b | 5180 | 6 | 3.51e−011 |
| 1741 | 18 | 238 | 346, 416 | hsa-miR-24 | 8071 | 6 | 1.22e−011 |
| 1742 | 18 | 256 | 441, 487 | hsa-miR-26a | 6472 | 6 | 2.28e−010 |
| 1743 | 18 | 259 | 481 | hsa-miR-27a | 1717 | 6 | 2.61e−009 |
| 1744 | 18 | 260 | 280 | hsa-miR-27b | 1257 | 6 | 1.35e−010 |
| 1745 | 18 | 167 | 556 | hsa-miR-29a | 1288 | 6 | 1.12e−011 |
| 1746 | 18 | 117 | 287 | hsa-miR-30a-3p | 529 | 6 | 3.99e−011 |
| 1747 | 18 | 244 | 287 | hsa-miR-30a-5p | 10843 | 6 | 3.2e−010 |
| 1748 | 18 | 242 | 311 | hsa-miR-30b | 648 | 6 | 4.13e−011 |
| 1749 | 18 | 243 | 299, 316 | hsa-miR-30c | 2968 | 6 | 1.16e−010 |
| 1750 | 18 | 241 | 550 | hsa-miR-30d | 3260 | 6 | 5.1e−011 |
| 1751 | 18 | 245 | 431 | hsa-miR-30e-5p | 1707 | 6 | 7.59e−011 |
| 1752 | 18 | 1 | 359 | hsa-miR-320 | 1149 | 6 | 4.5e−011 |
| 1753 | 18 | 113 | 538 | hsa-miR-328 | 662 | 6 | 9.64e−011 |
| 1754 | 18 | 141 | 437 | hsa-miR-33 | 1698 | 6 | 1.11e−010 |
| 1755 | 18 | 126 | 380 | hsa-miR-331 | 1304 | 6 | 2.71e−012 |
| 1756 | 18 | 194 | 337 | hsa-miR-338 | 1333 | 6 | 2.05e−011 |
| 1757 | 18 | 208 | 477 | hsa-miR-342 | 755 | 6 | 1.3e−010 |
| 1758 | 18 | 237 | 480 | hsa-miR-34a | 615 | 6 | 4.72e−012 |
| 1759 | 18 | 112 | 372 | hsa-miR-422b | 864 | 6 | 1.49e−009 |
| 1760 | 18 | 51 | 497 | hsa-miR-423 | 782 | 6 | 1.47e−011 |
| 1761 | 18 | 63 | 557 | hsa-miR-491 | 599 | 6 | 6.24e−009 |
| 1762 | 18 | 214 | 376 | hsa-miR-494 | 2281 | 6 | 6.65e−009 |
| 1763 | 18 | 83 | 312 | hsa-miR-497 | 1003 | 6 | 1.22e−011 |
| 1764 | 18 | 135 | 315 | hsa-miR-565 | 1354 | 6 | 7.69e−010 |
| 1765 | 18 | 60 | 479 | hsa-miR-638 | 1743 | 6 | 9.36e−009 |
| 1766 | 18 | 182 | 304, 317 | hsa-miR-92 | 2243 | 6 | 1.46e−009 |
| 1767 | 18 | 6 | 471 | hsa-miR-93 | 926 | 6 | 8.56e−011 |
| 1768 | 18 | 14 | 318 | hsa-miR-99a | 4186 | 6 | 1.86e−012 |
| 1769 | 18 | 80 | 509 | hsa-miR-99b | 717 | 6 | 5.79e−011 |
| 1770 | 19 | 127 | 361 | 11_1 | 635 | 9 | 7.26e−017 |
| 1771 | 19 | 196 | 508 | 15_rel | 23206 | 9 | 1.6e−016 |
| 1772 | 19 | 145 | 447 | 43_44_rel | 33792 | 4 | 4.72e−007 |
| 1773 | 19 | 94 | 394 | 69_6_rel | 1329 | 9 | 6.68e−016 |
| 1774 | 19 | 177 | 366, 464 | 72_12 | 961 | 9 | 2.29e−015 |
| 1775 | 19 | 33 | 339, 552 | 96_rel | 27130 | 4 | 3.84e−007 |
| 1776 | 19 | 87 | 301 | gam11 | 4427 | 9 | 2.51e−017 |
| 1777 | 19 | 223 | 320, 393, 559 | hsa-let-7a | 1971 | 9 | 4.05e−014 |
| 1778 | 19 | 223 | 320, 393, 559 | hsa-let-7a | 514 | 4 | 1.91e−005 |
| 1779 | 19 | 225 | 332 | hsa-let-7b | 10282 | 9 | 2.44e−017 |
| 1780 | 19 | 224 | 427 | hsa-let-7c | 3491 | 9 | 3.11e−015 |
| 1781 | 19 | 45 | 277 | hsa-let-7d | 1282 | 9 | 1.91e−016 |
| 1782 | 19 | 220 | 475 | hsa-let-7e | 1198 | 9 | 3.03e−017 |
| 1783 | 19 | 227 | 297 | hsa-let-7i | 515 | 9 | 1.78e−016 |
| 1784 | 19 | 13 | 397 | hsa-miR-100 | 804 | 9 | 3.51e−016 |
| 1785 | 19 | 48 | 483, 529 | hsa-miR-103 | 2676 | 9 | 8.49e−017 |
| 1786 | 19 | 184 | 423 | hsa-miR-105 | 580 | 9 | 1.65e−017 |
| 1787 | 19 | 2 | 399 | hsa-miR-106a | 725 | 9 | 8.64e−018 |
| 1788 | 19 | 47 | 555 | hsa-miR-107 | 1875 | 9 | 3.63e−016 |
| 1789 | 19 | 199 | 387 | hsa-miR-125a | 2762 | 9 | 1.33e−016 |
| 1790 | 19 | 198 | 313, 551 | hsa-miR-125b | 4418 | 9 | 3.62e−015 |
| 1791 | 19 | 204 | 419 | hsa-miR-126 | 1399 | 9 | 1.19e−016 |
| 1792 | 19 | 86 | 417 | hsa-miR-130a | 647 | 9 | 1.41e−017 |
| 1793 | 19 | 218 | 527 | hsa-miR-143 | 4649 | 9 | 9.8e−015 |
| 1794 | 19 | 139 | 281 | hsa-miR-145 | 3730 | 9 | 2.91e−016 |
| 1795 | 19 | 173 | 360, 468 | hsa-miR-16 | 1484 | 9 | 2.49e−015 |
| 1796 | 19 | 75 | 467 | hsa-miR-17-5p | 527 | 9 | 4.08e−016 |
| 1797 | 19 | 10 | 371, 461 | hsa-miR-181a | 1355 | 9 | 2.05e−015 |
| 1798 | 19 | 11 | 456, 540 | hsa-miR-181b | 505 | 9 | 5.62e−016 |
| 1799 | 19 | 77 | 440 | hsa-miR-191 | 823 | 9 | 5.25e−018 |
| 1800 | 19 | 17 | 442 | hsa-miR-193a | 1577 | 9 | 9.49e−018 |
| 1801 | 19 | 92 | 410, 432 | hsa-miR-199a | 1525 | 9 | 1.38e−015 |
| 1802 | 19 | 152 | 369 | hsa-miR-200a | 500 | 9 | 1.86e−014 |
| 1803 | 19 | 175 | 462 | hsa-miR-21 | 3024 | 9 | 1.73e−014 |
| 1804 | 19 | 115 | 314 | hsa-miR-210 | 1327 | 9 | 8.55e−014 |
| 1805 | 19 | 18 | 322 | hsa-miR-22 | 2121 | 9 | 4.34e−017 |
| 1806 | 19 | 50 | 364 | hsa-miR-221 | 1096 | 9 | 3.37e−018 |
| 1807 | 19 | 49 | 302 | hsa-miR-222 | 1244 | 9 | 2.42e−017 |
| 1808 | 19 | 67 | 336 | hsa-miR-23a | 3419 | 9 | 2.86e−017 |
| 1809 | 19 | 66 | 306 | hsa-miR-23b | 3242 | 9 | 3.99e−013 |
| 1810 | 19 | 238 | 346, 416 | hsa-miR-24 | 8989 | 9 | 7.92e−017 |
| 1811 | 19 | 256 | 441, 487 | hsa-miR-26a | 4957 | 9 | 2.76e−016 |
| 1812 | 19 | 259 | 481 | hsa-miR-27a | 2458 | 9 | 2.6e−017 |
| 1813 | 19 | 260 | 280 | hsa-miR-27b | 928 | 9 | 1.12e−015 |
| 1814 | 19 | 167 | 556 | hsa-miR-29a | 1057 | 9 | 1.6e−016 |
| 1815 | 19 | 244 | 287 | hsa-miR-30a-5p | 2404 | 9 | 1.16e−014 |
| 1816 | 19 | 243 | 299, 316 | hsa-miR-30c | 1173 | 9 | 7.29e−016 |
| 1817 | 19 | 241 | 550 | hsa-miR-30d | 1023 | 9 | 2.87e−014 |
| 1818 | 19 | 132 | 429 | hsa-miR-31 | 541 | 9 | 1.36e−012 |
| 1819 | 19 | 1 | 359 | hsa-miR-320 | 1095 | 9 | 3.84e−015 |
| 1820 | 19 | 141 | 437 | hsa-miR-33 | 786 | 9 | 1.15e−018 |
| 1821 | 19 | 126 | 380 | hsa-miR-331 | 686 | 9 | 1.41e−019 |
| 1822 | 19 | 194 | 337 | hsa-miR-338 | 1210 | 9 | 1.73e−016 |
| 1823 | 19 | 237 | 480 | hsa-miR-34a | 1472 | 9 | 1.76e−016 |
| 1824 | 19 | 51 | 497 | hsa-miR-423 | 778 | 9 | 5e−018 |
| 1825 | 19 | 214 | 376 | hsa-miR-494 | 1714 | 9 | 7.91e−014 |
| 1826 | 19 | 83 | 312 | hsa-miR-497 | 538 | 9 | 3.76e−017 |
| 1827 | 19 | 60 | 479 | hsa-miR-638 | 1367 | 9 | 4.66e−015 |
| 1828 | 19 | 36 | 385 | hsa-miR-766 | 1291 | 4 | 1.48e−006 |
| 1829 | 19 | 186 | 341 | hsa-miR-768-3p | 1195 | 4 | 1.39e−007 |
| 1830 | 19 | 182 | 304, 317 | hsa-miR-92 | 1138 | 9 | 1.01e−013 |
| 1831 | 19 | 6 | 471 | hsa-miR-93 | 700 | 9 | 5.66e−017 |
| 1832 | 19 | 14 | 318 | hsa-miR-99a | 1295 | 9 | 4.38e−015 |
| 1833 | 20 | 127 | 361 | 11_1 | 2138 | 8 | 2.36e−017 |
| 1834 | 20 | 196 | 508 | 15_rel | 14317 | 8 | 5.57e−016 |
| 1835 | 20 | 197 | 444 | 25_6 | 586 | 8 | 6.09e−019 |
| 1836 | 20 | 219 | 497 | 27_3 | 845 | 8 | 1.43e−016 |
| 1837 | 20 | 101 | 475 | 43_30 | 520 | 8 | 1.65e−019 |
| 1838 | 20 | 145 | 447 | 43_44_rel | 48688 | 8 | 2.84e−018 |
| 1839 | 20 | 114 | 530 | 43_6 | 504 | 8 | 2.93e−018 |
| 1840 | 20 | 94 | 394 | 69_6_rel | 1754 | 8 | 9.23e−017 |
| 1841 | 20 | 177 | 366, 464 | 72_12 | 2255 | 8 | 1.54e−016 |
| 1842 | 20 | 40 | 553 | 72_4 | 2039 | 8 | 3.9e−017 |
| 1843 | 20 | 33 | 339, 552 | 96_rel | 42707 | 8 | 1.94e−019 |
| 1844 | 20 | 87 | 301 | gam11 | 6156 | 8 | 2.53e−016 |
| 1845 | 20 | 125 | 532 | 70_16 | 531 | 8 | 1.05e−017 |
| 1846 | 20 | 223 | 320, 393, 559 | hsa-let-7a | 5443 | 8 | 2.45e−015 |
| 1847 | 20 | 223 | 320, 393, 559 | hsa-let-7a | 1688 | 8 | 1.1e−014 |
| 1848 | 20 | 225 | 332 | hsa-let-7b | 4996 | 8 | 2.93e−015 |
| 1849 | 20 | 224 | 427 | hsa-let-7c | 5038 | 8 | 8.02e−016 |
| 1850 | 20 | 45 | 277 | hsa-let-7d | 2457 | 8 | 2.76e−017 |
| 1851 | 20 | 220 | 475 | hsa-let-7e | 2293 | 8 | 1.6e−016 |
| 1852 | 20 | 222 | 482, 558 | hsa-let-7f | 641 | 8 | 1.29e−017 |
| 1853 | 20 | 226 | 395 | hsa-let-7g | 952 | 8 | 1.18e−018 |
| 1854 | 20 | 227 | 297 | hsa-let-7i | 1299 | 8 | 4.01e−018 |
| 1855 | 20 | 13 | 397 | hsa-miR-100 | 501 | 8 | 1.71e−017 |
| 1856 | 20 | 48 | 483, 529 | hsa-miR-103 | 1834 | 8 | 1.4e−015 |
| 1857 | 20 | 184 | 423 | hsa-miR-105 | 1977 | 8 | 2.59e−017 |
| 1858 | 20 | 2 | 399 | hsa-miR-106a | 1899 | 8 | 8.22e−015 |
| 1859 | 20 | 149 | 392 | hsa-miR-106b | 1848 | 8 | 8.63e−017 |
| 1860 | 20 | 47 | 555 | hsa-miR-107 | 1478 | 8 | 4.56e−016 |
| 1861 | 20 | 235 | 358 | hsa-miR-122a | 2614 | 8 | 3.63e−014 |
| 1862 | 20 | 199 | 387 | hsa-miR-125a | 2850 | 8 | 5.14e−016 |
| 1863 | 20 | 198 | 313, 551 | hsa-miR-125b | 2463 | 8 | 7.54e−017 |
| 1864 | 20 | 204 | 419 | hsa-miR-126 | 926 | 8 | 1.4e−016 |
| 1865 | 20 | 86 | 417 | hsa-miR-130a | 876 | 8 | 6.92e−017 |
| 1866 | 20 | 37 | 391 | hsa-miR-136 | 580 | 8 | 6.23e−018 |
| 1867 | 20 | 52 | 370, 548 | hsa-miR-138 | 762 | 8 | 2.85e−017 |
| 1868 | 20 | 64 | 402 | hsa-miR-140 | 701 | 8 | 1.19e−018 |
| 1869 | 20 | 151 | 472 | hsa-miR-141 | 1171 | 8 | 1.97e−014 |
| 1870 | 20 | 247 | 278 | hsa-miR-142-3p | 500 | 8 | 1.23e−017 |
| 1871 | 20 | 218 | 527 | hsa-miR-143 | 3451 | 8 | 1.2e−016 |
| 1872 | 20 | 139 | 281 | hsa-miR-145 | 1227 | 8 | 9.78e−016 |
| 1873 | 20 | 146 | 325 | hsa-miR-147 | 501 | 8 | 5.73e−018 |
| 1874 | 20 | 191 | 289 | hsa-miR-148b | 544 | 8 | 3.13e−017 |
| 1875 | 20 | 172 | 329 | hsa-miR-15b | 1539 | 8 | 3.21e−017 |
| 1876 | 20 | 173 | 360, 468 | hsa-miR-16 | 3359 | 8 | 5.53e−016 |
| 1877 | 20 | 38 | 467 | hsa-miR-17-3p | 696 | 8 | 5.13e−016 |
| 1878 | 20 | 75 | 467 | hsa-miR-17-5p | 2633 | 8 | 2.43e−016 |
| 1879 | 20 | 10 | 371, 461 | hsa-miR-181a | 2051 | 8 | 5.3e−016 |
| 1880 | 20 | 9 | 451 | hsa-miR-181c | 543 | 8 | 1.51e−018 |
| 1881 | 20 | 155 | 426 | hsa-miR-18a | 542 | 8 | 1.1e−015 |
| 1882 | 20 | 228 | 435 | hsa-miR-190 | 757 | 8 | 4.85e−017 |
| 1883 | 20 | 77 | 440 | hsa-miR-191 | 817 | 8 | 5.08e−016 |
| 1884 | 20 | 109 | 374 | hsa-miR-192 | 2196 | 8 | 2.27e−015 |
| 1885 | 20 | 17 | 442 | hsa-miR-193a | 2259 | 8 | 5.77e−015 |
| 1886 | 20 | 16 | 488 | hsa-miR-193b | 738 | 8 | 1.33e−017 |
| 1887 | 20 | 246 | 290, 549 | hsa-miR-194 | 3109 | 8 | 2.26e−015 |
| 1888 | 20 | 170 | 327 | hsa-miR-195 | 626 | 8 | 2.77e−016 |
| 1889 | 20 | 92 | 410, 432 | hsa-miR-199a | 2165 | 8 | 1.48e−016 |
| 1890 | 20 | 162 | 410, 432, 473 | hsa-miR-199a* | 928 | 8 | 7.6e−015 |
| 1891 | 20 | 251 | 282, 495 | hsa-miR-19b | 1563 | 8 | 1.03e−017 |
| 1892 | 20 | 152 | 369 | hsa-miR-200a | 1895 | 8 | 5.34e−016 |
| 1893 | 20 | 157 | 411 | hsa-miR-200b | 2121 | 8 | 8.51e−014 |
| 1894 | 20 | 156 | 446 | hsa-miR-200c | 3094 | 8 | 8.79e−015 |
| 1895 | 20 | 232 | 422 | hsa-miR-206 | 552 | 8 | 8.05e−018 |
| 1896 | 20 | 175 | 462 | hsa-miR-21 | 4077 | 8 | 8.13e−015 |
| 1897 | 20 | 115 | 314 | hsa-miR-210 | 751 | 8 | 2.64e−015 |
| 1898 | 20 | 29 | 439 | hsa-miR-214 | 925 | 8 | 1.02e−016 |
| 1899 | 20 | 18 | 322 | hsa-miR-22 | 1329 | 8 | 8.22e−016 |
| 1900 | 20 | 50 | 364 | hsa-miR-221 | 968 | 8 | 8.37e−015 |
| 1901 | 20 | 49 | 302 | hsa-miR-222 | 1288 | 8 | 9.55e−018 |
| 1902 | 20 | 248 | 415 | hsa-miR-223 | 679 | 8 | 6.24e−018 |
| 1903 | 20 | 67 | 336 | hsa-miR-23a | 4048 | 8 | 3.15e−016 |
| 1904 | 20 | 66 | 306 | hsa-miR-23b | 4954 | 8 | 2.51e−017 |
| 1905 | 20 | 238 | 346, 416 | hsa-miR-24 | 6899 | 8 | 5.55e−016 |
| 1906 | 20 | 90 | 388 | hsa-miR-25 | 592 | 8 | 2.56e−019 |
| 1907 | 20 | 256 | 441, 487 | hsa-miR-26a | 4352 | 8 | 2.73e−017 |
| 1908 | 20 | 259 | 481 | hsa-miR-27a | 2781 | 8 | 3.16e−016 |
| 1909 | 20 | 260 | 280 | hsa-miR-27b | 1168 | 8 | 1.16e−016 |
| 1910 | 20 | 61 | 286 | hsa-miR-296 | 1142 | 8 | 2.73e−018 |
| 1911 | 20 | 167 | 556 | hsa-miR-29a | 1628 | 8 | 1.91e−014 |
| 1912 | 20 | 168 | 303, 530 | hsa-miR-29b | 517 | 8 | 1.1e−017 |
| 1913 | 20 | 244 | 287 | hsa-miR-30a-5p | 561 | 8 | 6.06e−017 |
| 1914 | 20 | 242 | 311 | hsa-miR-30b | 544 | 8 | 4.77e−018 |
| 1915 | 20 | 243 | 299, 316 | hsa-miR-30c | 748 | 8 | 1e−014 |
| 1916 | 20 | 241 | 550 | hsa-miR-30d | 601 | 8 | 3e−013 |
| 1917 | 20 | 132 | 429 | hsa-miR-31 | 804 | 8 | 1.96e−015 |
| 1918 | 20 | 1 | 359 | hsa-miR-320 | 1237 | 8 | 3.75e−015 |
| 1919 | 20 | 113 | 538 | hsa-miR-328 | 874 | 8 | 1.06e−018 |
| 1920 | 20 | 141 | 437 | hsa-miR-33 | 1071 | 8 | 2.08e−019 |
| 1921 | 20 | 126 | 380 | hsa-miR-331 | 1042 | 8 | 1.77e−017 |
| 1922 | 20 | 194 | 337 | hsa-miR-338 | 1470 | 8 | 1.43e−016 |
| 1923 | 20 | 229 | 292 | hsa-miR-345 | 542 | 8 | 2.43e−016 |
| 1924 | 20 | 237 | 480 | hsa-miR-34a | 1129 | 8 | 4.55e−018 |
| 1925 | 20 | 128 | 294 | hsa-miR-370 | 689 | 8 | 2.13e−018 |
| 1926 | 20 | 273 | 335 | hsa-miR-375 | 565 | 8 | 8.59e−014 |
| 1927 | 20 | 112 | 372 | hsa-miR-422b | 527 | 8 | 3.28e−017 |
| 1928 | 20 | 51 | 497 | hsa-miR-423 | 1446 | 8 | 2.97e−017 |
| 1929 | 20 | 264 | 541 | hsa-miR-448 | 511 | 8 | 7.52e−017 |
| 1930 | 20 | 236 | 413 | hsa-miR-449 | 659 | 8 | 1.37e−018 |
| 1931 | 20 | 44 | 547 | hsa-miR-485-5p | 586 | 8 | 7.62e−018 |
| 1932 | 20 | 63 | 557 | hsa-miR-491 | 1769 | 8 | 7.27e−017 |
| 1933 | 20 | 54 | 293 | hsa-miR-492 | 542 | 8 | 1.04e−017 |
| 1934 | 20 | 214 | 376 | hsa-miR-494 | 17189 | 8 | 1.16e−016 |
| 1935 | 20 | 83 | 312 | hsa-miR-497 | 526 | 8 | 9.88e−016 |
| 1936 | 20 | 73 | 503 | hsa-miR-518b | 567 | 8 | 1.11e−018 |
| 1937 | 20 | 210 | 400 | hsa-miR-518c* | 543 | 8 | 3.06e−018 |
| 1938 | 20 | 206 | 513 | hsa-miR-520d* | 502 | 8 | 5.82e−019 |
| 1939 | 20 | 60 | 479 | hsa-miR-638 | 915 | 8 | 6.42e−014 |
| 1940 | 20 | 230 | 474, 484, 536 | hsa-miR-7 | 646 | 8 | 6.3e−018 |
| 1941 | 20 | 36 | 385 | hsa-miR-766 | 2213 | 8 | 8.42e−017 |
| 1942 | 20 | 186 | 341 | hsa-miR-768-3p | 3603 | 8 | 2.87e−016 |
| 1943 | 20 | 147 | 341 | hsa-miR-768-5p | 889 | 8 | 2.17e−015 |
| 1944 | 20 | 123 | 291 | hsa-miR-801 | 564 | 8 | 7.68e−016 |
| 1945 | 20 | 182 | 304, 317 | hsa-miR-92 | 2877 | 8 | 2.59e−016 |
| 1946 | 20 | 6 | 471 | hsa-miR-93 | 847 | 8 | 9.12e−016 |
| 1947 | 20 | 221 | 307 | hsa-miR-98 | 503 | 8 | 1.23e−017 |
| 1948 | 21 | 127 | 361 | 11_1 | 4493 | 31 | 0 |
| 1949 | 21 | 196 | 508 | 15_rel | 34427 | 31 | 0 |
| 1950 | 21 | 134 | 436 | 24_5_rel | 37892 | 11 | 3.83e−029 |
| 1951 | 21 | 219 | 497 | 27_3 | 1007 | 31 | 0 |
| 1952 | 21 | 79 | 460 | 43_37_rel | 6507 | 11 | 3.26e−024 |
| 1953 | 21 | 145 | 447 | 43_44_rel | 47020 | 13 | 1.68e−035 |
| 1954 | 21 | 94 | 394 | 69_6_rel | 3640 | 31 | 0 |
| 1955 | 21 | 177 | 366, 464 | 72_12 | 7536 | 31 | 0 |
| 1956 | 21 | 104 | 347 | 72_23 | 807 | 31 | 0 |
| 1957 | 21 | 40 | 553 | 72_4 | 1584 | 31 | 0 |
| 1958 | 21 | 97 | 357 | 74_26_rel | 780 | 31 | 0 |
| 1959 | 21 | 193 | 398 | 93_3_rel | 18814 | 11 | 1.89e−026 |
| 1960 | 21 | 33 | 339, 552 | 96_rel | 35764 | 13 | 9.69e−032 |
| 1961 | 21 | 87 | 301 | gam11 | 2791 | 31 | 0 |
| 1962 | 21 | 125 | 532 | 70_16 | 2142 | 31 | 0 |
| 1963 | 21 | 163 | 402 | 43_7 | 878 | 31 | 0 |
| 1964 | 21 | 223 | 320, 393, 559 | hsa-let-7a | 5021 | 31 | 0 |
| 1965 | 21 | 223 | 320, 393, 559 | hsa-let-7a | 630 | 10 | 7.71e−022 |
| 1966 | 21 | 225 | 332 | hsa-let-7b | 15296 | 31 | 0 |
| 1967 | 21 | 224 | 427 | hsa-let-7c | 11253 | 31 | 0 |
| 1968 | 21 | 45 | 277 | hsa-let-7d | 2184 | 31 | 0 |
| 1969 | 21 | 220 | 475 | hsa-let-7e | 2243 | 31 | 0 |
| 1970 | 21 | 226 | 395 | hsa-let-7g | 564 | 31 | 0 |
| 1971 | 21 | 227 | 297 | hsa-let-7i | 833 | 31 | 0 |
| 1972 | 21 | 13 | 397 | hsa-miR-100 | 2553 | 31 | 0 |
| 1973 | 21 | 48 | 483, 529 | hsa-miR-103 | 7559 | 31 | 0 |
| 1974 | 21 | 184 | 423 | hsa-miR-105 | 1799 | 31 | 0 |
| 1975 | 21 | 2 | 399 | hsa-miR-106a | 4058 | 31 | 0 |
| 1976 | 21 | 149 | 392 | hsa-miR-106b | 10802 | 31 | 0 |
| 1977 | 21 | 47 | 555 | hsa-miR-107 | 6864 | 31 | 0 |
| 1978 | 21 | 235 | 358 | hsa-miR-122a | 40164 | 31 | 0 |
| 1979 | 21 | 199 | 387 | hsa-miR-125a | 2880 | 31 | 0 |
| 1980 | 21 | 198 | 313, 551 | hsa-miR-125b | 12490 | 31 | 0 |
| 1981 | 21 | 204 | 419 | hsa-miR-126 | 8974 | 31 | 0 |
| 1982 | 21 | 86 | 417 | hsa-miR-130a | 2694 | 31 | 0 |
| 1983 | 21 | 218 | 527 | hsa-miR-143 | 2493 | 31 | 0 |
| 1984 | 21 | 139 | 281 | hsa-miR-145 | 3599 | 31 | 0 |
| 1985 | 21 | 190 | 458 | hsa-miR-148a | 658 | 31 | 0 |
| 1986 | 21 | 209 | 507 | hsa-miR-150 | 1003 | 31 | 0 |
| 1987 | 21 | 34 | 396 | hsa-miR-151 | 533 | 31 | 0 |
| 1988 | 21 | 192 | 470 | hsa-miR-152 | 596 | 31 | 0 |
| 1989 | 21 | 172 | 329 | hsa-miR-15b | 899 | 31 | 0 |
| 1990 | 21 | 173 | 360, 468 | hsa-miR-16 | 6596 | 31 | 0 |
| 1991 | 21 | 38 | 467 | hsa-miR-17-3p | 1430 | 31 | 0 |
| 1992 | 21 | 75 | 467 | hsa-miR-17-5p | 3063 | 31 | 0 |
| 1993 | 21 | 10 | 371, 461 | hsa-miR-181a | 2407 | 31 | 0 |
| 1994 | 21 | 11 | 456, 540 | hsa-miR-181b | 557 | 31 | 0 |
| 1995 | 21 | 234 | 450 | hsa-miR-185 | 1637 | 31 | 0 |
| 1996 | 21 | 77 | 440 | hsa-miR-191 | 3143 | 31 | 0 |
| 1997 | 21 | 109 | 374 | hsa-miR-192 | 5875 | 31 | 0 |
| 1998 | 21 | 17 | 442 | hsa-miR-193a | 9118 | 31 | 0 |
| 1999 | 21 | 16 | 488 | hsa-miR-193b | 1858 | 31 | 0 |
| 2000 | 21 | 246 | 290, 549 | hsa-miR-194 | 12264 | 31 | 0 |
| 2001 | 21 | 170 | 327 | hsa-miR-195 | 1292 | 31 | 0 |
| 2002 | 21 | 92 | 410, 432 | hsa-miR-199a | 11577 | 31 | 0 |
| 2003 | 21 | 162 | 410, 432, 473 | hsa-miR-199a* | 1500 | 31 | 0 |
| 2004 | 21 | 251 | 282, 495 | hsa-miR-19b | 2244 | 31 | 0 |
| 2005 | 21 | 157 | 411 | hsa-miR-200b | 597 | 31 | 0 |
| 2006 | 21 | 150 | 351 | hsa-miR-20a | 513 | 31 | 0 |
| 2007 | 21 | 175 | 462 | hsa-miR-21 | 4111 | 31 | 0 |
| 2008 | 21 | 29 | 439 | hsa-miR-214 | 1769 | 31 | 0 |
| 2009 | 21 | 18 | 322 | hsa-miR-22 | 11938 | 31 | 0 |
| 2010 | 21 | 50 | 364 | hsa-miR-221 | 1194 | 31 | 0 |
| 2011 | 21 | 49 | 302 | hsa-miR-222 | 1516 | 31 | 0 |
| 2012 | 21 | 67 | 336 | hsa-miR-23a | 5586 | 31 | 0 |
| 2013 | 21 | 66 | 306 | hsa-miR-23b | 3278 | 31 | 0 |
| 2014 | 21 | 238 | 346, 416 | hsa-miR-24 | 24310 | 31 | 0 |
| 2015 | 21 | 256 | 441, 487 | hsa-miR-26a | 18088 | 31 | 0 |
| 2016 | 21 | 259 | 481 | hsa-miR-27a | 5142 | 31 | 0 |
| 2017 | 21 | 260 | 280 | hsa-miR-27b | 3680 | 31 | 0 |
| 2018 | 21 | 61 | 286 | hsa-miR-296 | 916 | 31 | 0 |
| 2019 | 21 | 167 | 556 | hsa-miR-29a | 1028 | 31 | 0 |
| 2020 | 21 | 117 | 287 | hsa-miR-30a-3p | 523 | 31 | 0 |
| 2021 | 21 | 244 | 287 | hsa-miR-30a-5p | 1086 | 31 | 0 |
| 2022 | 21 | 242 | 311 | hsa-miR-30b | 1203 | 31 | 0 |
| 2023 | 21 | 243 | 299, 316 | hsa-miR-30c | 3232 | 31 | 0 |
| 2024 | 21 | 241 | 550 | hsa-miR-30d | 4046 | 31 | 0 |
| 2025 | 21 | 245 | 431 | hsa-miR-30e-5p | 4574 | 31 | 0 |
| 2026 | 21 | 1 | 359 | hsa-miR-320 | 3416 | 31 | 0 |
| 2027 | 21 | 91 | 368 | hsa-miR-324-3p | 627 | 31 | 0 |
| 2028 | 21 | 126 | 380 | hsa-miR-331 | 1294 | 31 | 0 |
| 2029 | 21 | 208 | 477 | hsa-miR-342 | 1297 | 31 | 0 |
| 2030 | 21 | 237 | 480 | hsa-miR-34a | 5247 | 31 | 0 |
| 2031 | 21 | 160 | 379, 382 | hsa-miR-365 | 575 | 31 | 0 |
| 2032 | 21 | 112 | 372 | hsa-miR-422b | 2482 | 31 | 0 |
| 2033 | 21 | 51 | 497 | hsa-miR-423 | 1360 | 31 | 0 |
| 2034 | 21 | 4 | 430 | hsa-miR-451 | 804 | 31 | 0 |
| 2035 | 21 | 63 | 557 | hsa-miR-491 | 835 | 31 | 0 |
| 2036 | 21 | 214 | 376 | hsa-miR-494 | 2149 | 31 | 0 |
| 2037 | 21 | 83 | 312 | hsa-miR-497 | 3108 | 31 | 0 |
| 2038 | 21 | 43 | 418 | hsa-miR-504 | 583 | 31 | 0 |
| 2039 | 21 | 135 | 315 | hsa-miR-565 | 1075 | 31 | 0 |
| 2040 | 21 | 30 | 285 | hsa-miR-622 | 779 | 31 | 0 |
| 2041 | 21 | 60 | 479 | hsa-miR-638 | 5851 | 31 | 0 |
| 2042 | 21 | 36 | 385 | hsa-miR-766 | 717 | 10 | 8.52e−023 |
| 2043 | 21 | 186 | 341 | hsa-miR-768-3p | 2790 | 10 | 8.88e−020 |
| 2044 | 21 | 147 | 341 | hsa-miR-768-5p | 1532 | 10 | 6.06e−024 |
| 2045 | 21 | 123 | 291 | hsa-miR-801 | 1534 | 10 | 4.32e−023 |
| 2046 | 21 | 182 | 304, 317 | hsa-miR-92 | 5390 | 31 | 0 |
| 2047 | 21 | 6 | 471 | hsa-miR-93 | 1768 | 31 | 0 |
| 2048 | 21 | 14 | 318 | hsa-miR-99a | 8365 | 31 | 0 |
| 2049 | 21 | 131 | 430 | 6_11 | 833 | 31 | 0 |
| 2050 | 22 | 127 | 361 | 11_1 | 5042 | 32 | 0 |
| 2051 | 22 | 196 | 508 | 15_rel | 32711 | 32 | 0 |
| 2052 | 22 | 134 | 436 | 24_5_rel | 41761 | 13 | 4.34e−033 |
| 2053 | 22 | 219 | 497 | 27_3 | 1261 | 32 | 0 |
| 2054 | 22 | 79 | 460 | 43_37_rel | 11726 | 13 | 5.34e−030 |
| 2055 | 22 | 145 | 447 | 43_44_rel | 44239 | 15 | 0 |
| 2056 | 22 | 94 | 394 | 69_6_rel | 7062 | 32 | 0 |
| 2057 | 22 | 177 | 366, 464 | 72_12 | 4287 | 32 | 0 |
| 2058 | 22 | 40 | 553 | 72_4 | 2063 | 32 | 0 |
| 2059 | 22 | 97 | 357 | 74_26_rel | 731 | 32 | 0 |
| 2060 | 22 | 193 | 398 | 93_3_rel | 29492 | 13 | 2.88e−032 |
| 2061 | 22 | 33 | 339, 552 | 96_rel | 41483 | 15 | 0 |
| 2062 | 22 | 87 | 301 | gam11 | 3897 | 32 | 0 |
| 2063 | 22 | 125 | 532 | 70_16 | 2160 | 32 | 0 |
| 2064 | 22 | 163 | 402 | 43_7 | 1247 | 32 | 0 |
| 2065 | 22 | 223 | 320, 393, 559 | hsa-let-7a | 7450 | 32 | 0 |
| 2066 | 22 | 223 | 320, 393, 559 | hsa-let-7a | 1171 | 9 | 7.09e−018 |
| 2067 | 22 | 225 | 332 | hsa-let-7b | 11828 | 32 | 0 |
| 2068 | 22 | 224 | 427 | hsa-let-7c | 8507 | 32 | 0 |
| 2069 | 22 | 45 | 277 | hsa-let-7d | 3115 | 32 | 0 |
| 2070 | 22 | 220 | 475 | hsa-let-7e | 2072 | 32 | 0 |
| 2071 | 22 | 226 | 395 | hsa-let-7g | 635 | 32 | 0 |
| 2072 | 22 | 227 | 297 | hsa-let-7i | 793 | 32 | 0 |
| 2073 | 22 | 13 | 397 | hsa-miR-100 | 1356 | 32 | 0 |
| 2074 | 22 | 48 | 483, 529 | hsa-miR-103 | 9503 | 32 | 0 |
| 2075 | 22 | 184 | 423 | hsa-miR-105 | 2946 | 32 | 0 |
| 2076 | 22 | 2 | 399 | hsa-miR-106a | 4097 | 32 | 0 |
| 2077 | 22 | 149 | 392 | hsa-miR-106b | 7745 | 32 | 0 |
| 2078 | 22 | 47 | 555 | hsa-miR-107 | 9082 | 32 | 0 |
| 2079 | 22 | 235 | 358 | hsa-miR-122a | 24639 | 32 | 0 |
| 2080 | 22 | 199 | 387 | hsa-miR-125a | 1674 | 32 | 0 |
| 2081 | 22 | 198 | 313, 551 | hsa-miR-125b | 8221 | 32 | 0 |
| 2082 | 22 | 204 | 419 | hsa-miR-126 | 7059 | 32 | 0 |
| 2083 | 22 | 86 | 417 | hsa-miR-130a | 1430 | 32 | 0 |
| 2084 | 22 | 218 | 527 | hsa-miR-143 | 2284 | 32 | 0 |
| 2085 | 22 | 139 | 281 | hsa-miR-145 | 2286 | 32 | 0 |
| 2086 | 22 | 190 | 458 | hsa-miR-148a | 599 | 32 | 0 |
| 2087 | 22 | 209 | 507 | hsa-miR-150 | 503 | 32 | 0 |
| 2088 | 22 | 34 | 396 | hsa-miR-151 | 593 | 32 | 0 |
| 2089 | 22 | 192 | 470 | hsa-miR-152 | 627 | 32 | 0 |
| 2090 | 22 | 172 | 329 | hsa-miR-15b | 2233 | 32 | 0 |
| 2091 | 22 | 173 | 360, 468 | hsa-miR-16 | 7155 | 32 | 0 |
| 2092 | 22 | 38 | 467 | hsa-miR-17-3p | 1489 | 32 | 0 |
| 2093 | 22 | 75 | 467 | hsa-miR-17-5p | 3058 | 32 | 0 |
| 2094 | 22 | 10 | 371, 461 | hsa-miR-181a | 2629 | 32 | 0 |
| 2095 | 22 | 11 | 456, 540 | hsa-miR-181b | 791 | 32 | 0 |
| 2096 | 22 | 234 | 450 | hsa-miR-185 | 1811 | 32 | 0 |
| 2097 | 22 | 155 | 426 | hsa-miR-18a | 642 | 32 | 0 |
| 2098 | 22 | 77 | 440 | hsa-miR-191 | 3963 | 32 | 0 |
| 2099 | 22 | 109 | 374 | hsa-miR-192 | 4289 | 32 | 0 |
| 2100 | 22 | 17 | 442 | hsa-miR-193a | 10843 | 32 | 0 |
| 2101 | 22 | 16 | 488 | hsa-miR-193b | 1680 | 32 | 0 |
| 2102 | 22 | 246 | 290, 549 | hsa-miR-194 | 7540 | 32 | 0 |
| 2103 | 22 | 170 | 327 | hsa-miR-195 | 830 | 32 | 0 |
| 2104 | 22 | 92 | 410, 432 | hsa-miR-199a | 2704 | 32 | 0 |
| 2105 | 22 | 162 | 410, 432, 473 | hsa-miR-199a* | 710 | 32 | 0 |
| 2106 | 22 | 251 | 282, 495 | hsa-miR-19b | 2226 | 32 | 0 |
| 2107 | 22 | 175 | 462 | hsa-miR-21 | 9644 | 32 | 0 |
| 2108 | 22 | 115 | 314 | hsa-miR-210 | 678 | 32 | 0 |
| 2109 | 22 | 29 | 439 | hsa-miR-214 | 719 | 32 | 0 |
| 2110 | 22 | 18 | 322 | hsa-miR-22 | 6192 | 32 | 0 |
| 2111 | 22 | 50 | 364 | hsa-miR-221 | 2960 | 32 | 0 |
| 2112 | 22 | 49 | 302 | hsa-miR-222 | 3838 | 32 | 0 |
| 2113 | 22 | 67 | 336 | hsa-miR-23a | 4986 | 32 | 0 |
| 2114 | 22 | 66 | 306 | hsa-miR-23b | 3765 | 32 | 0 |
| 2115 | 22 | 238 | 346, 416 | hsa-miR-24 | 25334 | 32 | 0 |
| 2116 | 22 | 90 | 388 | hsa-miR-25 | 771 | 32 | 0 |
| 2117 | 22 | 256 | 441, 487 | hsa-miR-26a | 16168 | 32 | 0 |
| 2118 | 22 | 259 | 481 | hsa-miR-27a | 4292 | 32 | 0 |
| 2119 | 22 | 260 | 280 | hsa-miR-27b | 4412 | 32 | 0 |
| 2120 | 22 | 61 | 286 | hsa-miR-296 | 634 | 32 | 0 |
| 2121 | 22 | 167 | 556 | hsa-miR-29a | 1016 | 32 | 0 |
| 2122 | 22 | 244 | 287 | hsa-miR-30a-5p | 757 | 32 | 0 |
| 2123 | 22 | 242 | 311 | hsa-miR-30b | 1483 | 32 | 0 |
| 2124 | 22 | 243 | 299, 316 | hsa-miR-30c | 3304 | 32 | 0 |
| 2125 | 22 | 241 | 550 | hsa-miR-30d | 4381 | 32 | 0 |
| 2126 | 22 | 245 | 431 | hsa-miR-30e-5p | 3412 | 32 | 0 |
| 2127 | 22 | 1 | 359 | hsa-miR-320 | 3795 | 32 | 0 |
| 2128 | 22 | 91 | 368 | hsa-miR-324-3p | 529 | 32 | 0 |
| 2129 | 22 | 126 | 380 | hsa-miR-331 | 2146 | 32 | 0 |
| 2130 | 22 | 200 | 448 | hsa-miR-339 | 900 | 32 | 0 |
| 2131 | 22 | 208 | 477 | hsa-miR-342 | 1209 | 32 | 0 |
| 2132 | 22 | 229 | 292 | hsa-miR-345 | 692 | 32 | 0 |
| 2133 | 22 | 237 | 480 | hsa-miR-34a | 5015 | 32 | 0 |
| 2134 | 22 | 255 | 444 | hsa-miR-361 | 539 | 32 | 0 |
| 2135 | 22 | 112 | 372 | hsa-miR-422b | 875 | 32 | 0 |
| 2136 | 22 | 51 | 497 | hsa-miR-423 | 1875 | 32 | 0 |
| 2137 | 22 | 63 | 557 | hsa-miR-491 | 1212 | 32 | 0 |
| 2138 | 22 | 214 | 376 | hsa-miR-494 | 2311 | 32 | 0 |
| 2139 | 22 | 83 | 312 | hsa-miR-497 | 1507 | 32 | 0 |
| 2140 | 22 | 135 | 315 | hsa-miR-565 | 824 | 32 | 0 |
| 2141 | 22 | 60 | 479 | hsa-miR-638 | 9745 | 32 | 0 |
| 2142 | 22 | 36 | 385 | hsa-miR-766 | 502 | 9 | 2.96e−018 |
| 2143 | 22 | 186 | 341 | hsa-miR-768-3p | 2355 | 9 | 1.01e−016 |
| 2144 | 22 | 147 | 341 | hsa-miR-768-5p | 2304 | 9 | 1.65e−021 |
| 2145 | 22 | 123 | 291 | hsa-miR-801 | 3466 | 9 | 1.57e−021 |
| 2146 | 22 | 182 | 304, 317 | hsa-miR-92 | 5049 | 32 | 0 |
| 2147 | 22 | 6 | 471 | hsa-miR-93 | 2713 | 32 | 0 |
| 2148 | 22 | 14 | 318 | hsa-miR-99a | 3646 | 32 | 0 |
| 2149 | 22 | 131 | 430 | 6_11 | 545 | 32 | 0 |
| 2150 | 23 | 196 | 508 | 15_rel | 42199 | 7 | 3.82e−017 |
| 2151 | 23 | 219 | 497 | 27_3 | 1030 | 6 | 4.5e−013 |
| 2152 | 23 | 129 | 548 | 43_39 | 512 | 6 | 3.1e−013 |
| 2153 | 23 | 145 | 447 | 43_44_rel | 47668 | 6 | 4.46e−014 |
| 2154 | 23 | 94 | 394 | 69_6_rel | 5947 | 7 | 7.79e−016 |
| 2155 | 23 | 177 | 366, 464 | 72_12 | 1300 | 7 | 1.75e−016 |
| 2156 | 23 | 40 | 553 | 72_4 | 764 | 7 | 2.79e−014 |
| 2157 | 23 | 97 | 357 | 74_26_rel | 954 | 7 | 2.08e−016 |
| 2158 | 23 | 33 | 339, 552 | 96_rel | 27424 | 7 | 2.14e−017 |
| 2159 | 23 | 87 | 301 | gam11 | 12013 | 7 | 3.13e−017 |
| 2160 | 23 | 223 | 320, 393, 559 | hsa-let-7a | 2656 | 7 | 8.52e−015 |
| 2161 | 23 | 223 | 320, 393, 559 | hsa-let-7a | 689 | 5 | 3.79e−009 |
| 2162 | 23 | 225 | 332 | hsa-let-7b | 12705 | 7 | 6.92e−018 |
| 2163 | 23 | 224 | 427 | hsa-let-7c | 4316 | 7 | 2.48e−015 |
| 2164 | 23 | 45 | 277 | hsa-let-7d | 1682 | 7 | 7.99e−018 |
| 2165 | 23 | 220 | 475 | hsa-let-7e | 1728 | 7 | 1.71e−018 |
| 2166 | 23 | 226 | 395 | hsa-let-7g | 752 | 7 | 1.18e−016 |
| 2167 | 23 | 227 | 297 | hsa-let-7i | 1327 | 7 | 9.09e−017 |
| 2168 | 23 | 13 | 397 | hsa-miR-100 | 1602 | 7 | 7.75e−017 |
| 2169 | 23 | 48 | 483, 529 | hsa-miR-103 | 2026 | 7 | 6.1e−018 |
| 2170 | 23 | 184 | 423 | hsa-miR-105 | 513 | 7 | 6.04e−015 |
| 2171 | 23 | 2 | 399 | hsa-miR-106a | 951 | 7 | 1.76e−016 |
| 2172 | 23 | 149 | 392 | hsa-miR-106b | 1063 | 7 | 2.14e−016 |
| 2173 | 23 | 47 | 555 | hsa-miR-107 | 1615 | 7 | 2.61e−017 |
| 2174 | 23 | 199 | 387 | hsa-miR-125a | 2215 | 7 | 3.69e−017 |
| 2175 | 23 | 198 | 313, 551 | hsa-miR-125b | 4617 | 7 | 6.35e−017 |
| 2176 | 23 | 204 | 419 | hsa-miR-126 | 2439 | 7 | 5.45e−016 |
| 2177 | 23 | 89 | 419 | hsa-miR-126* | 607 | 7 | 5.24e−015 |
| 2178 | 23 | 86 | 417 | hsa-miR-130a | 990 | 7 | 1.01e−016 |
| 2179 | 23 | 151 | 472 | hsa-miR-141 | 2986 | 7 | 7.13e−016 |
| 2180 | 23 | 218 | 527 | hsa-miR-143 | 4168 | 7 | 1.9e−016 |
| 2181 | 23 | 139 | 281 | hsa-miR-145 | 3851 | 7 | 6.5e−017 |
| 2182 | 23 | 217 | 466 | hsa-miR-146a | 529 | 7 | 3.61e−017 |
| 2183 | 23 | 209 | 507 | hsa-miR-150 | 1092 | 7 | 5.51e−017 |
| 2184 | 23 | 173 | 360, 468 | hsa-miR-16 | 2374 | 7 | 1.25e−014 |
| 2185 | 23 | 75 | 467 | hsa-miR-17-5p | 745 | 7 | 1.53e−016 |
| 2186 | 23 | 10 | 371, 461 | hsa-miR-181a | 3138 | 7 | 3.38e−017 |
| 2187 | 23 | 11 | 456, 540 | hsa-miR-181b | 804 | 7 | 3.92e−017 |
| 2188 | 23 | 234 | 450 | hsa-miR-185 | 578 | 7 | 3.13e−018 |
| 2189 | 23 | 77 | 440 | hsa-miR-191 | 1442 | 7 | 2.36e−016 |
| 2190 | 23 | 130 | 440 | hsa-miR-191* | 561 | 7 | 3.56e−016 |
| 2191 | 23 | 17 | 442 | hsa-miR-193a | 1004 | 7 | 7.31e−017 |
| 2192 | 23 | 16 | 488 | hsa-miR-193b | 688 | 7 | 1.89e−017 |
| 2193 | 23 | 170 | 327 | hsa-miR-195 | 558 | 7 | 1.46e−016 |
| 2194 | 23 | 92 | 410, 432 | hsa-miR-199a | 2967 | 7 | 1.39e−017 |
| 2195 | 23 | 162 | 410, 432, 473 | hsa-miR-199a* | 596 | 7 | 3.21e−015 |
| 2196 | 23 | 251 | 282, 495 | hsa-miR-19b | 653 | 7 | 6.57e−016 |
| 2197 | 23 | 152 | 369 | hsa-miR-200a | 869 | 7 | 5.91e−016 |
| 2198 | 23 | 157 | 411 | hsa-miR-200b | 943 | 7 | 5.41e−016 |
| 2199 | 23 | 156 | 446 | hsa-miR-200c | 3213 | 7 | 1.67e−016 |
| 2200 | 23 | 175 | 462 | hsa-miR-21 | 6763 | 7 | 5.23e−014 |
| 2201 | 23 | 115 | 314 | hsa-miR-210 | 843 | 7 | 3.55e−016 |
| 2202 | 23 | 29 | 439 | hsa-miR-214 | 1196 | 7 | 5.95e−016 |
| 2203 | 23 | 18 | 322 | hsa-miR-22 | 2699 | 7 | 9.13e−017 |
| 2204 | 23 | 50 | 364 | hsa-miR-221 | 2882 | 7 | 1.24e−016 |
| 2205 | 23 | 49 | 302 | hsa-miR-222 | 2880 | 7 | 2.5e−017 |
| 2206 | 23 | 67 | 336 | hsa-miR-23a | 3070 | 7 | 2.47e−015 |
| 2207 | 23 | 66 | 306 | hsa-miR-23b | 2324 | 7 | 5.42e−013 |
| 2208 | 23 | 238 | 346, 416 | hsa-miR-24 | 9252 | 7 | 5.21e−017 |
| 2209 | 23 | 90 | 388 | hsa-miR-25 | 632 | 7 | 8.05e−017 |
| 2210 | 23 | 256 | 441, 487 | hsa-miR-26a | 5625 | 7 | 1.68e−015 |
| 2211 | 23 | 259 | 481 | hsa-miR-27a | 3037 | 7 | 4.74e−015 |
| 2212 | 23 | 260 | 280 | hsa-miR-27b | 734 | 7 | 5.13e−014 |
| 2213 | 23 | 61 | 286 | hsa-miR-296 | 1276 | 7 | 6.91e−015 |
| 2214 | 23 | 167 | 556 | hsa-miR-29a | 1898 | 7 | 1.5e−014 |
| 2215 | 23 | 244 | 287 | hsa-miR-30a-5p | 1642 | 7 | 4.1e−018 |
| 2216 | 23 | 242 | 311 | hsa-miR-30b | 626 | 7 | 5.47e−015 |
| 2217 | 23 | 243 | 299, 316 | hsa-miR-30c | 936 | 7 | 2.07e−015 |
| 2218 | 23 | 241 | 550 | hsa-miR-30d | 2315 | 7 | 6.89e−016 |
| 2219 | 23 | 245 | 431 | hsa-miR-30e-5p | 1006 | 7 | 7.89e−016 |
| 2220 | 23 | 132 | 429 | hsa-miR-31 | 1204 | 7 | 3.38e−015 |
| 2221 | 23 | 1 | 359 | hsa-miR-320 | 2648 | 7 | 6.44e−018 |
| 2222 | 23 | 91 | 368 | hsa-miR-324-3p | 659 | 7 | 2.25e−015 |
| 2223 | 23 | 113 | 538 | hsa-miR-328 | 744 | 7 | 4.06e−017 |
| 2224 | 23 | 141 | 437 | hsa-miR-33 | 762 | 7 | 2.41e−015 |
| 2225 | 23 | 126 | 380 | hsa-miR-331 | 857 | 7 | 4.62e−017 |
| 2226 | 23 | 194 | 337 | hsa-miR-338 | 667 | 7 | 1.72e−013 |
| 2227 | 23 | 208 | 477 | hsa-miR-342 | 695 | 7 | 1.95e−017 |
| 2228 | 23 | 237 | 480 | hsa-miR-34a | 2305 | 7 | 7.89e−016 |
| 2229 | 23 | 128 | 294 | hsa-miR-370 | 630 | 7 | 1.81e−014 |
| 2230 | 23 | 112 | 372 | hsa-miR-422b | 527 | 7 | 1.05e−015 |
| 2231 | 23 | 51 | 497 | hsa-miR-423 | 1453 | 7 | 3.01e−017 |
| 2232 | 23 | 236 | 413 | hsa-miR-449 | 763 | 7 | 2.38e−015 |
| 2233 | 23 | 63 | 557 | hsa-miR-491 | 1402 | 7 | 6.33e−015 |
| 2234 | 23 | 214 | 376 | hsa-miR-494 | 5899 | 7 | 6.79e−015 |
| 2235 | 23 | 83 | 312 | hsa-miR-497 | 730 | 7 | 1.01e−016 |
| 2236 | 23 | 103 | 342 | hsa-miR-525 | 528 | 7 | 8.51e−015 |
| 2237 | 23 | 135 | 315 | hsa-miR-565 | 1503 | 7 | 1.5e−015 |
| 2238 | 23 | 60 | 479 | hsa-miR-638 | 5915 | 7 | 2.12e−015 |
| 2239 | 23 | 59 | 330 | hsa-miR-663 | 595 | 7 | 3.38e−014 |
| 2240 | 23 | 186 | 341 | hsa-miR-768-3p | 1745 | 6 | 1.54e−012 |
| 2241 | 23 | 147 | 341 | hsa-miR-768-5p | 533 | 5 | 1.02e−011 |
| 2242 | 23 | 123 | 291 | hsa-miR-801 | 1205 | 6 | 1.05e−013 |
| 2243 | 23 | 182 | 304, 317 | hsa-miR-92 | 1828 | 7 | 2.03e−017 |
| 2244 | 23 | 6 | 471 | hsa-miR-93 | 1346 | 7 | 8.98e−016 |
| 2245 | 23 | 14 | 318 | hsa-miR-99a | 1039 | 7 | 1.22e−017 |
| 2246 | 23 | 80 | 509 | hsa-miR-99b | 714 | 7 | 5.15e−019 |
| 2247 | 24 | 196 | 508 | 15_rel | 43110 | 5 | 8.93e−013 |
| 2248 | 24 | 219 | 497 | 27_3 | 821 | 5 | 2.63e−012 |
| 2249 | 24 | 129 | 548 | 43_39 | 519 | 5 | 4.89e−011 |
| 2250 | 24 | 145 | 447 | 43_44_rel | 41428 | 3 | 2.75e−007 |
| 2251 | 24 | 94 | 394 | 69_6_rel | 9845 | 5 | 2.51e−011 |
| 2252 | 24 | 97 | 357 | 74_26_rel | 1457 | 5 | 1.85e−011 |
| 2253 | 24 | 33 | 339, 552 | 96_rel | 32148 | 3 | 3.26e−007 |
| 2254 | 24 | 87 | 301 | gam11 | 7017 | 5 | 2.18e−010 |
| 2255 | 24 | 225 | 332 | hsa-let-7b | 1773 | 5 | 7.41e−011 |
| 2256 | 24 | 224 | 427 | hsa-let-7c | 790 | 5 | 6.46e−012 |
| 2257 | 24 | 220 | 475 | hsa-let-7e | 956 | 5 | 1.14e−011 |
| 2258 | 24 | 226 | 395 | hsa-let-7g | 771 | 5 | 4.68e−011 |
| 2259 | 24 | 227 | 297 | hsa-let-7i | 526 | 5 | 3.08e−012 |
| 2260 | 24 | 13 | 397 | hsa-miR-100 | 726 | 5 | 1.9e−010 |
| 2261 | 24 | 198 | 313, 551 | hsa-miR-125b | 1515 | 5 | 3.11e−010 |
| 2262 | 24 | 86 | 417 | hsa-miR-130a | 1002 | 5 | 5.07e−011 |
| 2263 | 24 | 151 | 472 | hsa-miR-141 | 837 | 5 | 7.37e−011 |
| 2264 | 24 | 218 | 527 | hsa-miR-143 | 627 | 5 | 2.75e−012 |
| 2265 | 24 | 139 | 281 | hsa-miR-145 | 604 | 5 | 1.11e−010 |
| 2266 | 24 | 10 | 371, 461 | hsa-miR-181a | 1001 | 5 | 5.55e−011 |
| 2267 | 24 | 179 | 356 | hsa-miR-183 | 902 | 5 | 2.95e−011 |
| 2268 | 24 | 16 | 488 | hsa-miR-193b | 549 | 5 | 2.63e−011 |
| 2269 | 24 | 156 | 446 | hsa-miR-200c | 905 | 5 | 1.24e−011 |
| 2270 | 24 | 175 | 462 | hsa-miR-21 | 2077 | 5 | 9.11e−011 |
| 2271 | 24 | 115 | 314 | hsa-miR-210 | 664 | 5 | 2.53e−011 |
| 2272 | 24 | 29 | 439 | hsa-miR-214 | 584 | 5 | 7.81e−012 |
| 2273 | 24 | 18 | 322 | hsa-miR-22 | 1095 | 5 | 1.54e−010 |
| 2274 | 24 | 50 | 364 | hsa-miR-221 | 753 | 5 | 2.57e−010 |
| 2275 | 24 | 49 | 302 | hsa-miR-222 | 1219 | 5 | 7.77e−011 |
| 2276 | 24 | 238 | 346, 416 | hsa-miR-24 | 1555 | 5 | 1.32e−010 |
| 2277 | 24 | 256 | 441, 487 | hsa-miR-26a | 757 | 5 | 1.41e−011 |
| 2278 | 24 | 61 | 286 | hsa-miR-296 | 1425 | 5 | 5.61e−011 |
| 2279 | 24 | 132 | 429 | hsa-miR-31 | 768 | 5 | 5.65e−011 |
| 2280 | 24 | 1 | 359 | hsa-miR-320 | 1386 | 5 | 3.09e−011 |
| 2281 | 24 | 113 | 538 | hsa-miR-328 | 685 | 5 | 2.44e−012 |
| 2282 | 24 | 141 | 437 | hsa-miR-33 | 642 | 5 | 9.93e−012 |
| 2283 | 24 | 237 | 480 | hsa-miR-34a | 803 | 5 | 1.37e−012 |
| 2284 | 24 | 51 | 497 | hsa-miR-423 | 1700 | 5 | 4.1e−011 |
| 2285 | 24 | 236 | 413 | hsa-miR-449 | 2130 | 5 | 1.78e−010 |
| 2286 | 24 | 44 | 547 | hsa-miR-485-5p | 506 | 5 | 3.21e−012 |
| 2287 | 24 | 63 | 557 | hsa-miR-491 | 1828 | 5 | 8.86e−011 |
| 2288 | 24 | 214 | 376 | hsa-miR-494 | 3991 | 5 | 2.39e−010 |
| 2289 | 24 | 258 | 428, 443 | hsa-miR-513 | 583 | 5 | 4.88e−011 |
| 2290 | 24 | 60 | 479 | hsa-miR-638 | 9867 | 5 | 9.7e−011 |
| 2291 | 24 | 182 | 304, 317 | hsa-miR-92 | 752 | 5 | 1.65e−011 |
| 2292 | 25 | 127 | 361 | 11_1 | 511 | 15 | 5.28e−035 |
| 2293 | 25 | 196 | 508 | 15_rel | 34962 | 13 | 3.8e−030 |
| 2294 | 25 | 134 | 436 | 24_5_rel | 37322 | 4 | 2e−010 |
| 2295 | 25 | 219 | 497 | 27_3 | 1102 | 15 | 0 |
| 2296 | 25 | 79 | 460 | 43_37_rel | 27512 | 4 | 2.64e−009 |
| 2297 | 25 | 129 | 548 | 43_39 | 849 | 15 | 5.92e−035 |
| 2298 | 25 | 145 | 447 | 43_44_rel | 44753 | 7 | 1.23e−017 |
| 2299 | 25 | 94 | 394 | 69_6_rel | 7301 | 13 | 1.38e−030 |
| 2300 | 25 | 177 | 366, 464 | 72_12 | 1665 | 13 | 3.45e−032 |
| 2301 | 25 | 40 | 553 | 72_4 | 1083 | 13 | 8.69e−034 |
| 2302 | 25 | 97 | 357 | 74_26_rel | 1430 | 13 | 5.85e−029 |
| 2303 | 25 | 193 | 398 | 93_3_rel | 32958 | 4 | 2.21e−009 |
| 2304 | 25 | 33 | 339, 552 | 96_rel | 34397 | 7 | 1.21e−018 |
| 2305 | 25 | 87 | 301 | gam11 | 8987 | 13 | 4.99e−027 |
| 2306 | 25 | 163 | 402 | 43_7 | 1175 | 13 | 2.72e−030 |
| 2307 | 25 | 223 | 320, 393, 559 | hsa-let-7a | 5329 | 14 | 6.18e−037 |
| 2308 | 25 | 223 | 320, 393, 559 | hsa-let-7a | 1800 | 4 | 1.56e−008 |
| 2309 | 25 | 225 | 332 | hsa-let-7b | 14146 | 15 | 0 |
| 2310 | 25 | 224 | 427 | hsa-let-7c | 8089 | 14 | 1.24e−037 |
| 2311 | 25 | 45 | 277 | hsa-let-7d | 2138 | 15 | 0 |
| 2312 | 25 | 220 | 475 | hsa-let-7e | 1953 | 14 | 0 |
| 2313 | 25 | 226 | 395 | hsa-let-7g | 734 | 14 | 1.44e−033 |
| 2314 | 25 | 227 | 297 | hsa-let-7i | 1294 | 15 | 0 |
| 2315 | 25 | 13 | 397 | hsa-miR-100 | 1755 | 15 | 0 |
| 2316 | 25 | 48 | 483, 529 | hsa-miR-103 | 2014 | 15 | 9.13e−037 |
| 2317 | 25 | 184 | 423 | hsa-miR-105 | 1023 | 14 | 0 |
| 2318 | 25 | 2 | 399 | hsa-miR-106a | 1349 | 15 | 0 |
| 2319 | 25 | 149 | 392 | hsa-miR-106b | 996 | 14 | 0 |
| 2320 | 25 | 47 | 555 | hsa-miR-107 | 1747 | 15 | 2.46e−037 |
| 2321 | 25 | 199 | 387 | hsa-miR-125a | 3006 | 14 | 1.22e−035 |
| 2322 | 25 | 198 | 313, 551 | hsa-miR-125b | 6882 | 15 | 0 |
| 2323 | 25 | 204 | 419 | hsa-miR-126 | 7421 | 14 | 1.1e−032 |
| 2324 | 25 | 86 | 417 | hsa-miR-130a | 1998 | 15 | 0 |
| 2325 | 25 | 151 | 472 | hsa-miR-141 | 2276 | 15 | 4.21e−037 |
| 2326 | 25 | 218 | 527 | hsa-miR-143 | 5834 | 14 | 6.83e−030 |
| 2327 | 25 | 139 | 281 | hsa-miR-145 | 6209 | 14 | 1.83e−035 |
| 2328 | 25 | 212 | 333 | hsa-miR-149 | 528 | 15 | 3.06e−038 |
| 2329 | 25 | 209 | 507 | hsa-miR-150 | 915 | 14 | 1.74e−036 |
| 2330 | 25 | 172 | 329 | hsa-miR-15b | 1045 | 15 | 0 |
| 2331 | 25 | 173 | 360, 468 | hsa-miR-16 | 5990 | 14 | 5.3e−035 |
| 2332 | 25 | 75 | 467 | hsa-miR-17-5p | 1194 | 14 | 1.33e−035 |
| 2333 | 25 | 10 | 371, 461 | hsa-miR-181a | 2868 | 14 | 9.38e−034 |
| 2334 | 25 | 11 | 456, 540 | hsa-miR-181b | 603 | 15 | 0 |
| 2335 | 25 | 234 | 450 | hsa-miR-185 | 877 | 15 | 6.88e−038 |
| 2336 | 25 | 77 | 440 | hsa-miR-191 | 1412 | 15 | 0 |
| 2337 | 25 | 17 | 442 | hsa-miR-193a | 2184 | 15 | 0 |
| 2338 | 25 | 16 | 488 | hsa-miR-193b | 515 | 15 | 8.89e−036 |
| 2339 | 25 | 170 | 327 | hsa-miR-195 | 1092 | 15 | 0 |
| 2340 | 25 | 92 | 410, 432 | hsa-miR-199a | 3127 | 14 | 1.59e−036 |
| 2341 | 25 | 162 | 410, 432, 473 | hsa-miR-199a* | 688 | 15 | 3.2e−037 |
| 2342 | 25 | 251 | 282, 495 | hsa-miR-19b | 1030 | 14 | 3.52e−032 |
| 2343 | 25 | 152 | 369 | hsa-miR-200a | 508 | 14 | 2.88e−033 |
| 2344 | 25 | 157 | 411 | hsa-miR-200b | 924 | 15 | 0 |
| 2345 | 25 | 156 | 446 | hsa-miR-200c | 2932 | 15 | 3.84e−038 |
| 2346 | 25 | 203 | 504 | hsa-miR-205 | 640 | 15 | 1.48e−032 |
| 2347 | 25 | 175 | 462 | hsa-miR-21 | 3686 | 15 | 9.2e−038 |
| 2348 | 25 | 29 | 439 | hsa-miR-214 | 1087 | 15 | 0 |
| 2349 | 25 | 18 | 322 | hsa-miR-22 | 3568 | 15 | 1.49e−038 |
| 2350 | 25 | 50 | 364 | hsa-miR-221 | 2244 | 15 | 1.97e−038 |
| 2351 | 25 | 49 | 302 | hsa-miR-222 | 2451 | 15 | 0 |
| 2352 | 25 | 67 | 336 | hsa-miR-23a | 4908 | 15 | 0 |
| 2353 | 25 | 66 | 306 | hsa-miR-23b | 1918 | 15 | 2.43e−029 |
| 2354 | 25 | 238 | 346, 416 | hsa-miR-24 | 10999 | 15 | 0 |
| 2355 | 25 | 256 | 441, 487 | hsa-miR-26a | 9168 | 15 | 0 |
| 2356 | 25 | 259 | 481 | hsa-miR-27a | 5941 | 15 | 0 |
| 2357 | 25 | 260 | 280 | hsa-miR-27b | 1840 | 15 | 0 |
| 2358 | 25 | 61 | 286 | hsa-miR-296 | 1095 | 15 | 7.54e−033 |
| 2359 | 25 | 167 | 556 | hsa-miR-29a | 1110 | 15 | 1.87e−031 |
| 2360 | 25 | 244 | 287 | hsa-miR-30a-5p | 1464 | 15 | 1.3e−030 |
| 2361 | 25 | 242 | 311 | hsa-miR-30b | 1247 | 15 | 3.4e−037 |
| 2362 | 25 | 243 | 299, 316 | hsa-miR-30c | 1834 | 15 | 0 |
| 2363 | 25 | 241 | 550 | hsa-miR-30d | 2948 | 15 | 3e−037 |
| 2364 | 25 | 245 | 431 | hsa-miR-30e-5p | 1434 | 15 | 0 |
| 2365 | 25 | 1 | 359 | hsa-miR-320 | 2374 | 15 | 0 |
| 2366 | 25 | 91 | 368 | hsa-miR-324-3p | 649 | 15 | 0 |
| 2367 | 25 | 98 | 368 | hsa-miR-324-5p | 514 | 15 | 3.99e−038 |
| 2368 | 25 | 113 | 538 | hsa-miR-328 | 817 | 15 | 0 |
| 2369 | 25 | 141 | 437 | hsa-miR-33 | 770 | 15 | 3.43e−035 |
| 2370 | 25 | 126 | 380 | hsa-miR-331 | 672 | 15 | 1.44e−038 |
| 2371 | 25 | 208 | 477 | hsa-miR-342 | 926 | 15 | 0 |
| 2372 | 25 | 237 | 480 | hsa-miR-34a | 2293 | 15 | 0 |
| 2373 | 25 | 57 | 498 | hsa-miR-34c | 973 | 15 | 6.23e−037 |
| 2374 | 25 | 160 | 379, 382 | hsa-miR-365 | 555 | 15 | 0 |
| 2375 | 25 | 51 | 497 | hsa-miR-423 | 1820 | 14 | 2.91e−037 |
| 2376 | 25 | 236 | 413 | hsa-miR-449 | 1261 | 15 | 2.68e−034 |
| 2377 | 25 | 4 | 430 | hsa-miR-451 | 928 | 13 | 7.94e−029 |
| 2378 | 25 | 63 | 557 | hsa-miR-491 | 1732 | 15 | 0 |
| 2379 | 25 | 214 | 376 | hsa-miR-494 | 9153 | 15 | 4.07e−036 |
| 2380 | 25 | 83 | 312 | hsa-miR-497 | 1632 | 15 | 0 |
| 2381 | 25 | 258 | 428, 443 | hsa-miR-513 | 607 | 15 | 2.92e−034 |
| 2382 | 25 | 135 | 315 | hsa-miR-565 | 1791 | 13 | 2.77e−031 |
| 2383 | 25 | 60 | 479 | hsa-miR-638 | 7942 | 13 | 2.6e−030 |
| 2384 | 25 | 186 | 341 | hsa-miR-768-3p | 3344 | 3 | 7.41e−007 |
| 2385 | 25 | 147 | 341 | hsa-miR-768-5p | 735 | 3 | 4.46e−007 |
| 2386 | 25 | 123 | 291 | hsa-miR-801 | 1372 | 3 | 3.75e−007 |
| 2387 | 25 | 182 | 304, 317 | hsa-miR-92 | 2086 | 14 | 1.01e−034 |
| 2388 | 25 | 6 | 471 | hsa-miR-93 | 1229 | 15 | 0 |
| 2389 | 25 | 14 | 318 | hsa-miR-99a | 2161 | 15 | 0 |
| 2390 | 25 | 80 | 509 | hsa-miR-99b | 920 | 14 | 1.43e−037 |
| 2391 | 25 | 131 | 430 | 6_11 | 1549 | 15 | 3.1e−035 |
| 2392 | 26 | 196 | 508 | 15_rel | 37566 | 14 | 7.96e−038 |
| 2393 | 26 | 134 | 436 | 24_5_rel | 33292 | 11 | 7.41e−030 |
| 2394 | 26 | 219 | 497 | 27_3 | 513 | 4 | 1.72e−008 |
| 2395 | 26 | 79 | 460 | 43_37_rel | 15084 | 11 | 7.59e−025 |
| 2396 | 26 | 129 | 548 | 43_39 | 645 | 4 | 1.13e−009 |
| 2397 | 26 | 145 | 447 | 43_44_rel | 24960 | 3 | 4.02e−007 |
| 2398 | 26 | 94 | 394 | 69_6_rel | 6641 | 14 | 1.91e−036 |
| 2399 | 26 | 40 | 553 | 72_4 | 734 | 14 | 7.4e−034 |
| 2400 | 26 | 97 | 357 | 74_26_rel | 1155 | 14 | 1.46e−034 |
| 2401 | 26 | 33 | 339, 552 | 96_rel | 9581 | 13 | 1.78e−031 |
| 2402 | 26 | 87 | 301 | gam11 | 9954 | 14 | 1.52e−034 |
| 2403 | 26 | 223 | 320, 393, 559 | hsa-let-7a | 840 | 14 | 4.3e−030 |
| 2404 | 26 | 225 | 332 | hsa-let-7b | 6052 | 14 | 1.16e−034 |
| 2405 | 26 | 224 | 427 | hsa-let-7c | 1901 | 14 | 1.87e−033 |
| 2406 | 26 | 45 | 277 | hsa-let-7d | 748 | 14 | 9.51e−034 |
| 2407 | 26 | 220 | 475 | hsa-let-7e | 825 | 14 | 1.34e−037 |
| 2408 | 26 | 226 | 395 | hsa-let-7g | 674 | 14 | 1.87e−037 |
| 2409 | 26 | 227 | 297 | hsa-let-7i | 783 | 14 | 1.12e−033 |
| 2410 | 26 | 13 | 397 | hsa-miR-100 | 633 | 14 | 1.04e−035 |
| 2411 | 26 | 48 | 483, 529 | hsa-miR-103 | 797 | 14 | 1.83e−034 |
| 2412 | 26 | 149 | 392 | hsa-miR-106b | 621 | 14 | 5.45e−036 |
| 2413 | 26 | 47 | 555 | hsa-miR-107 | 767 | 14 | 2.11e−034 |
| 2414 | 26 | 199 | 387 | hsa-miR-125a | 777 | 14 | 8.09e−036 |
| 2415 | 26 | 198 | 313, 551 | hsa-miR-125b | 1777 | 14 | 8.56e−038 |
| 2416 | 26 | 204 | 419 | hsa-miR-126 | 690 | 14 | 8.22e−034 |
| 2417 | 26 | 89 | 419 | hsa-miR-126* | 628 | 14 | 1.85e−033 |
| 2418 | 26 | 151 | 472 | hsa-miR-141 | 2018 | 14 | 9.45e−034 |
| 2419 | 26 | 218 | 527 | hsa-miR-143 | 1587 | 14 | 1.91e−034 |
| 2420 | 26 | 139 | 281 | hsa-miR-145 | 2342 | 14 | 6.55e−037 |
| 2421 | 26 | 173 | 360, 468 | hsa-miR-16 | 697 | 14 | 2.03e−032 |
| 2422 | 26 | 10 | 371, 461 | hsa-miR-181a | 1220 | 14 | 2.53e−036 |
| 2423 | 26 | 77 | 440 | hsa-miR-191 | 925 | 14 | 5.38e−038 |
| 2424 | 26 | 17 | 442 | hsa-miR-193a | 537 | 14 | 2.9e−035 |
| 2425 | 26 | 92 | 410, 432 | hsa-miR-199a | 1074 | 14 | 2.45e−038 |
| 2426 | 26 | 152 | 369 | hsa-miR-200a | 736 | 14 | 3e−036 |
| 2427 | 26 | 157 | 411 | hsa-miR-200b | 785 | 14 | 3.46e−033 |
| 2428 | 26 | 156 | 446 | hsa-miR-200c | 3078 | 14 | 2.56e−035 |
| 2429 | 26 | 203 | 504 | hsa-miR-205 | 3155 | 14 | 7.42e−029 |
| 2430 | 26 | 175 | 462 | hsa-miR-21 | 3170 | 14 | 5.51e−030 |
| 2431 | 26 | 18 | 322 | hsa-miR-22 | 1519 | 14 | 2.52e−035 |
| 2432 | 26 | 50 | 364 | hsa-miR-221 | 967 | 14 | 3.08e−036 |
| 2433 | 26 | 49 | 302 | hsa-miR-222 | 1067 | 14 | 2.12e−035 |
| 2434 | 26 | 67 | 336 | hsa-miR-23a | 1602 | 14 | 3.24e−034 |
| 2435 | 26 | 66 | 306 | hsa-miR-23b | 1419 | 14 | 1.01e−033 |
| 2436 | 26 | 238 | 346, 416 | hsa-miR-24 | 2802 | 14 | 2e−036 |
| 2437 | 26 | 90 | 388 | hsa-miR-25 | 609 | 14 | 1.42e−034 |
| 2438 | 26 | 256 | 441, 487 | hsa-miR-26a | 1312 | 14 | 3.45e−034 |
| 2439 | 26 | 259 | 481 | hsa-miR-27a | 1333 | 14 | 5.54e−035 |
| 2440 | 26 | 61 | 286 | hsa-miR-296 | 635 | 14 | 5.61e−036 |
| 2441 | 26 | 244 | 287 | hsa-miR-30a-5p | 872 | 14 | 2.6e−033 |
| 2442 | 26 | 241 | 550 | hsa-miR-30d | 1130 | 14 | 9.82e−035 |
| 2443 | 26 | 245 | 431 | hsa-miR-30e-5p | 651 | 14 | 9.76e−035 |
| 2444 | 26 | 132 | 429 | hsa-miR-31 | 768 | 14 | 7.92e−032 |
| 2445 | 26 | 1 | 359 | hsa-miR-320 | 1293 | 14 | 2.17e−037 |
| 2446 | 26 | 237 | 480 | hsa-miR-34a | 687 | 14 | 1.79e−035 |
| 2447 | 26 | 128 | 294 | hsa-miR-370 | 510 | 14 | 2.7e−034 |
| 2448 | 26 | 111 | 389 | hsa-miR-422a | 745 | 14 | 8.27e−031 |
| 2449 | 26 | 112 | 372 | hsa-miR-422b | 1002 | 14 | 1.74e−031 |
| 2450 | 26 | 51 | 497 | hsa-miR-423 | 887 | 14 | 1.28e−036 |
| 2451 | 26 | 63 | 557 | hsa-miR-491 | 952 | 14 | 1.34e−035 |
| 2452 | 26 | 214 | 376 | hsa-miR-494 | 2924 | 14 | 1.16e−032 |
| 2453 | 26 | 258 | 428, 443 | hsa-miR-513 | 585 | 14 | 2.16e−036 |
| 2454 | 26 | 135 | 315 | hsa-miR-565 | 1531 | 14 | 8.67e−034 |
| 2455 | 26 | 60 | 479 | hsa-miR-638 | 4414 | 14 | 1.15e−035 |
| 2456 | 26 | 186 | 341 | hsa-miR-768-3p | 893 | 12 | 5.01e−031 |
| 2457 | 26 | 123 | 291 | hsa-miR-801 | 1039 | 12 | 1.46e−029 |
| 2458 | 26 | 182 | 304, 317 | hsa-miR-92 | 1407 | 14 | 5.6e−036 |
| 2459 | 26 | 6 | 471 | hsa-miR-93 | 909 | 14 | 3.92e−035 |
| 2460 | 26 | 14 | 318 | hsa-miR-99a | 693 | 14 | 0 |
| 2461 | 27 | 196 | 508 | 15_rel | 44421 | 37 | 0 |
| 2462 | 27 | 134 | 436 | 24_5_rel | 42349 | 20 | 0 |
| 2463 | 27 | 219 | 497 | 27_3 | 1033 | 24 | 0 |
| 2464 | 27 | 79 | 460 | 43_37_rel | 25134 | 20 | 0 |
| 2465 | 27 | 129 | 548 | 43_39 | 578 | 24 | 0 |
| 2466 | 27 | 145 | 447 | 43_44_rel | 37772 | 14 | 7.94e−037 |
| 2467 | 27 | 94 | 394 | 69_6_rel | 8535 | 37 | 0 |
| 2468 | 27 | 177 | 366, 464 | 72_12 | 746 | 37 | 0 |
| 2469 | 27 | 40 | 553 | 72_4 | 1077 | 37 | 0 |
| 2470 | 27 | 97 | 357 | 74_26_rel | 1434 | 37 | 0 |
| 2471 | 27 | 33 | 339, 552 | 96_rel | 19509 | 28 | 0 |
| 2472 | 27 | 87 | 301 | gam11 | 13038 | 37 | 0 |
| 2473 | 27 | 223 | 320, 393, 559 | hsa-let-7a | 1413 | 38 | 0 |
| 2474 | 27 | 223 | 320, 393, 559 | hsa-let-7a | 775 | 8 | 4.18e−016 |
| 2475 | 27 | 225 | 332 | hsa-let-7b | 8296 | 38 | 0 |
| 2476 | 27 | 224 | 427 | hsa-let-7c | 2786 | 38 | 0 |
| 2477 | 27 | 45 | 277 | hsa-let-7d | 1089 | 38 | 0 |
| 2478 | 27 | 220 | 475 | hsa-let-7e | 1266 | 38 | 0 |
| 2479 | 27 | 226 | 395 | hsa-let-7g | 761 | 38 | 0 |
| 2480 | 27 | 227 | 297 | hsa-let-7i | 987 | 38 | 0 |
| 2481 | 27 | 13 | 397 | hsa-miR-100 | 896 | 38 | 0 |
| 2482 | 27 | 48 | 483, 529 | hsa-miR-103 | 1244 | 38 | 0 |
| 2483 | 27 | 2 | 399 | hsa-miR-106a | 706 | 38 | 0 |
| 2484 | 27 | 149 | 392 | hsa-miR-106b | 832 | 38 | 0 |
| 2485 | 27 | 47 | 555 | hsa-miR-107 | 1124 | 38 | 0 |
| 2486 | 27 | 199 | 387 | hsa-miR-125a | 1161 | 38 | 0 |
| 2487 | 27 | 198 | 313, 551 | hsa-miR-125b | 2637 | 38 | 0 |
| 2488 | 27 | 204 | 419 | hsa-miR-126 | 975 | 38 | 0 |
| 2489 | 27 | 89 | 419 | hsa-miR-126* | 653 | 38 | 0 |
| 2490 | 27 | 86 | 417 | hsa-miR-130a | 810 | 38 | 0 |
| 2491 | 27 | 151 | 472 | hsa-miR-141 | 2972 | 38 | 0 |
| 2492 | 27 | 218 | 527 | hsa-miR-143 | 1619 | 38 | 0 |
| 2493 | 27 | 139 | 281 | hsa-miR-145 | 2431 | 38 | 0 |
| 2494 | 27 | 209 | 507 | hsa-miR-150 | 540 | 38 | 0 |
| 2495 | 27 | 173 | 360, 468 | hsa-miR-16 | 1189 | 38 | 0 |
| 2496 | 27 | 75 | 467 | hsa-miR-17-5p | 571 | 38 | 0 |
| 2497 | 27 | 10 | 371, 461 | hsa-miR-181a | 1863 | 38 | 0 |
| 2498 | 27 | 11 | 456, 540 | hsa-miR-181b | 523 | 38 | 0 |
| 2499 | 27 | 77 | 440 | hsa-miR-191 | 1256 | 38 | 0 |
| 2500 | 27 | 17 | 442 | hsa-miR-193a | 908 | 38 | 0 |
| 2501 | 27 | 16 | 488 | hsa-miR-193b | 654 | 38 | 0 |
| 2502 | 27 | 92 | 410, 432 | hsa-miR-199a | 1531 | 38 | 0 |
| 2503 | 27 | 152 | 369 | hsa-miR-200a | 692 | 38 | 0 |
| 2504 | 27 | 157 | 411 | hsa-miR-200b | 1005 | 38 | 0 |
| 2505 | 27 | 156 | 446 | hsa-miR-200c | 3891 | 38 | 0 |
| 2506 | 27 | 203 | 504 | hsa-miR-205 | 1429 | 38 | 0 |
| 2507 | 27 | 175 | 462 | hsa-miR-21 | 4123 | 38 | 0 |
| 2508 | 27 | 115 | 314 | hsa-miR-210 | 722 | 38 | 0 |
| 2509 | 27 | 29 | 439 | hsa-miR-214 | 777 | 38 | 0 |
| 2510 | 27 | 18 | 322 | hsa-miR-22 | 1860 | 38 | 0 |
| 2511 | 27 | 50 | 364 | hsa-miR-221 | 1327 | 38 | 0 |
| 2512 | 27 | 49 | 302 | hsa-miR-222 | 1664 | 38 | 0 |
| 2513 | 27 | 67 | 336 | hsa-miR-23a | 1947 | 38 | 0 |
| 2514 | 27 | 66 | 306 | hsa-miR-23b | 1227 | 38 | 0 |
| 2515 | 27 | 238 | 346, 416 | hsa-miR-24 | 4710 | 38 | 0 |
| 2516 | 27 | 90 | 388 | hsa-miR-25 | 647 | 38 | 0 |
| 2517 | 27 | 256 | 441, 487 | hsa-miR-26a | 2398 | 38 | 0 |
| 2518 | 27 | 259 | 481 | hsa-miR-27a | 1710 | 38 | 0 |
| 2519 | 27 | 260 | 280 | hsa-miR-27b | 564 | 38 | 0 |
| 2520 | 27 | 61 | 286 | hsa-miR-296 | 783 | 38 | 0 |
| 2521 | 27 | 167 | 556 | hsa-miR-29a | 534 | 38 | 0 |
| 2522 | 27 | 244 | 287 | hsa-miR-30a-5p | 624 | 38 | 0 |
| 2523 | 27 | 243 | 299, 316 | hsa-miR-30c | 504 | 38 | 0 |
| 2524 | 27 | 241 | 550 | hsa-miR-30d | 1366 | 38 | 0 |
| 2525 | 27 | 245 | 431 | hsa-miR-30e-5p | 808 | 38 | 0 |
| 2526 | 27 | 132 | 429 | hsa-miR-31 | 877 | 38 | 0 |
| 2527 | 27 | 1 | 359 | hsa-miR-320 | 1873 | 38 | 0 |
| 2528 | 27 | 113 | 538 | hsa-miR-328 | 629 | 38 | 0 |
| 2529 | 27 | 126 | 380 | hsa-miR-331 | 549 | 38 | 0 |
| 2530 | 27 | 208 | 477 | hsa-miR-342 | 528 | 38 | 0 |
| 2531 | 27 | 237 | 480 | hsa-miR-34a | 1155 | 38 | 0 |
| 2532 | 27 | 128 | 294 | hsa-miR-370 | 660 | 38 | 0 |
| 2533 | 27 | 112 | 372 | hsa-miR-422b | 732 | 38 | 0 |
| 2534 | 27 | 51 | 497 | hsa-miR-423 | 1498 | 38 | 0 |
| 2535 | 27 | 236 | 413 | hsa-miR-449 | 953 | 38 | 0 |
| 2536 | 27 | 44 | 547 | hsa-miR-485-5p | 544 | 38 | 0 |
| 2537 | 27 | 63 | 557 | hsa-miR-491 | 1627 | 38 | 0 |
| 2538 | 27 | 214 | 376 | hsa-miR-494 | 6263 | 38 | 0 |
| 2539 | 27 | 83 | 312 | hsa-miR-497 | 516 | 38 | 0 |
| 2540 | 27 | 258 | 428, 443 | hsa-miR-513 | 729 | 38 | 0 |
| 2541 | 27 | 135 | 315 | hsa-miR-565 | 1801 | 37 | 0 |
| 2542 | 27 | 60 | 479 | hsa-miR-638 | 6638 | 37 | 0 |
| 2543 | 27 | 186 | 341 | hsa-miR-768-3p | 1520 | 22 | 0 |
| 2544 | 27 | 123 | 291 | hsa-miR-801 | 1368 | 22 | 0 |
| 2545 | 27 | 182 | 304, 317 | hsa-miR-92 | 1826 | 38 | 0 |
| 2546 | 27 | 6 | 471 | hsa-miR-93 | 1228 | 38 | 0 |
| 2547 | 27 | 14 | 318 | hsa-miR-99a | 789 | 38 | 0 |
| 2548 | 27 | 80 | 509 | hsa-miR-99b | 510 | 38 | 0 |
| 2549 | 28 | 196 | 508 | 15_rel | 53669 | 19 | 0 |
| 2550 | 28 | 134 | 436 | 24_5_rel | 44212 | 10 | 3.59e−027 |
| 2551 | 28 | 219 | 497 | 27_3 | 1123 | 15 | 1.64e−037 |
| 2552 | 28 | 79 | 460 | 43_37_rel | 26957 | 10 | 7.21e−023 |
| 2553 | 28 | 129 | 548 | 43_39 | 666 | 15 | 1.12e−035 |
| 2554 | 28 | 145 | 447 | 43_44_rel | 38035 | 7 | 1.48e−020 |
| 2555 | 28 | 94 | 394 | 69_6_rel | 11622 | 19 | 0 |
| 2556 | 28 | 177 | 366, 464 | 72_12 | 842 | 19 | 0 |
| 2557 | 28 | 40 | 553 | 72_4 | 1313 | 19 | 0 |
| 2558 | 28 | 97 | 357 | 74_26_rel | 1783 | 19 | 0 |
| 2559 | 28 | 193 | 398 | 93_3_rel | 1371 | 10 | 4.06e−017 |
| 2560 | 28 | 33 | 339, 552 | 96_rel | 25646 | 12 | 3.12e−029 |
| 2561 | 28 | 87 | 301 | gam11 | 17521 | 19 | 0 |
| 2562 | 28 | 223 | 320, 393, 559 | hsa-let-7a | 1759 | 20 | 0 |
| 2563 | 28 | 225 | 332 | hsa-let-7b | 8751 | 20 | 0 |
| 2564 | 28 | 224 | 427 | hsa-let-7c | 3057 | 20 | 0 |
| 2565 | 28 | 45 | 277 | hsa-let-7d | 1312 | 20 | 0 |
| 2566 | 28 | 220 | 475 | hsa-let-7e | 1461 | 20 | 0 |
| 2567 | 28 | 226 | 395 | hsa-let-7g | 805 | 20 | 0 |
| 2568 | 28 | 227 | 297 | hsa-let-7i | 1117 | 20 | 0 |
| 2569 | 28 | 13 | 397 | hsa-miR-100 | 874 | 20 | 0 |
| 2570 | 28 | 48 | 483, 529 | hsa-miR-103 | 1420 | 20 | 0 |
| 2571 | 28 | 2 | 399 | hsa-miR-106a | 1015 | 20 | 0 |
| 2572 | 28 | 149 | 392 | hsa-miR-106b | 999 | 20 | 0 |
| 2573 | 28 | 47 | 555 | hsa-miR-107 | 1265 | 20 | 0 |
| 2574 | 28 | 199 | 387 | hsa-miR-125a | 1050 | 20 | 0 |
| 2575 | 28 | 198 | 313, 551 | hsa-miR-125b | 2572 | 20 | 0 |
| 2576 | 28 | 204 | 419 | hsa-miR-126 | 979 | 20 | 0 |
| 2577 | 28 | 89 | 419 | hsa-miR-126* | 649 | 20 | 0 |
| 2578 | 28 | 86 | 417 | hsa-miR-130a | 1054 | 20 | 0 |
| 2579 | 28 | 151 | 472 | hsa-miR-141 | 4244 | 20 | 0 |
| 2580 | 28 | 218 | 527 | hsa-miR-143 | 1588 | 20 | 0 |
| 2581 | 28 | 139 | 281 | hsa-miR-145 | 2250 | 20 | 0 |
| 2582 | 28 | 173 | 360, 468 | hsa-miR-16 | 1349 | 20 | 0 |
| 2583 | 28 | 75 | 467 | hsa-miR-17-5p | 775 | 20 | 0 |
| 2584 | 28 | 10 | 371, 461 | hsa-miR-181a | 1894 | 20 | 0 |
| 2585 | 28 | 179 | 356 | hsa-miR-183 | 818 | 20 | 0 |
| 2586 | 28 | 77 | 440 | hsa-miR-191 | 1384 | 20 | 0 |
| 2587 | 28 | 17 | 442 | hsa-miR-193a | 1000 | 20 | 0 |
| 2588 | 28 | 16 | 488 | hsa-miR-193b | 758 | 20 | 0 |
| 2589 | 28 | 92 | 410, 432 | hsa-miR-199a | 1411 | 20 | 0 |
| 2590 | 28 | 152 | 369 | hsa-miR-200a | 740 | 20 | 0 |
| 2591 | 28 | 157 | 411 | hsa-miR-200b | 1315 | 20 | 0 |
| 2592 | 28 | 156 | 446 | hsa-miR-200c | 5011 | 20 | 0 |
| 2593 | 28 | 203 | 504 | hsa-miR-205 | 1407 | 20 | 0 |
| 2594 | 28 | 175 | 462 | hsa-miR-21 | 6030 | 20 | 0 |
| 2595 | 28 | 115 | 314 | hsa-miR-210 | 847 | 20 | 0 |
| 2596 | 28 | 29 | 439 | hsa-miR-214 | 751 | 20 | 0 |
| 2597 | 28 | 18 | 322 | hsa-miR-22 | 2069 | 20 | 0 |
| 2598 | 28 | 50 | 364 | hsa-miR-221 | 1259 | 20 | 0 |
| 2599 | 28 | 49 | 302 | hsa-miR-222 | 1754 | 20 | 0 |
| 2600 | 28 | 67 | 336 | hsa-miR-23a | 1952 | 20 | 0 |
| 2601 | 28 | 66 | 306 | hsa-miR-23b | 1434 | 20 | 0 |
| 2602 | 28 | 238 | 346, 416 | hsa-miR-24 | 5270 | 20 | 0 |
| 2603 | 28 | 90 | 388 | hsa-miR-25 | 704 | 20 | 0 |
| 2604 | 28 | 256 | 441, 487 | hsa-miR-26a | 2585 | 20 | 0 |
| 2605 | 28 | 259 | 481 | hsa-miR-27a | 1740 | 20 | 0 |
| 2606 | 28 | 260 | 280 | hsa-miR-27b | 598 | 20 | 0 |
| 2607 | 28 | 61 | 286 | hsa-miR-296 | 807 | 20 | 0 |
| 2608 | 28 | 244 | 287 | hsa-miR-30a-5p | 515 | 20 | 0 |
| 2609 | 28 | 241 | 550 | hsa-miR-30d | 1311 | 20 | 0 |
| 2610 | 28 | 245 | 431 | hsa-miR-30e-5p | 917 | 20 | 0 |
| 2611 | 28 | 132 | 429 | hsa-miR-31 | 891 | 20 | 0 |
| 2612 | 28 | 1 | 359 | hsa-miR-320 | 1997 | 20 | 0 |
| 2613 | 28 | 113 | 538 | hsa-miR-328 | 698 | 20 | 0 |
| 2614 | 28 | 141 | 437 | hsa-miR-33 | 546 | 20 | 0 |
| 2615 | 28 | 126 | 380 | hsa-miR-331 | 527 | 20 | 0 |
| 2616 | 28 | 208 | 477 | hsa-miR-342 | 560 | 20 | 0 |
| 2617 | 28 | 229 | 292 | hsa-miR-345 | 509 | 20 | 0 |
| 2618 | 28 | 237 | 480 | hsa-miR-34a | 1254 | 20 | 0 |
| 2619 | 28 | 128 | 294 | hsa-miR-370 | 637 | 20 | 0 |
| 2620 | 28 | 112 | 372 | hsa-miR-422b | 617 | 20 | 0 |
| 2621 | 28 | 51 | 497 | hsa-miR-423 | 1913 | 20 | 0 |
| 2622 | 28 | 236 | 413 | hsa-miR-449 | 1480 | 20 | 0 |
| 2623 | 28 | 44 | 547 | hsa-miR-485-5p | 515 | 20 | 0 |
| 2624 | 28 | 63 | 557 | hsa-miR-491 | 2329 | 20 | 0 |
| 2625 | 28 | 214 | 376 | hsa-miR-494 | 9710 | 20 | 0 |
| 2626 | 28 | 258 | 428, 443 | hsa-miR-513 | 848 | 20 | 0 |
| 2627 | 28 | 135 | 315 | hsa-miR-565 | 2230 | 19 | 0 |
| 2628 | 28 | 60 | 479 | hsa-miR-638 | 9161 | 19 | 0 |
| 2629 | 28 | 186 | 341 | hsa-miR-768-3p | 2056 | 7 | 1.64e−015 |
| 2630 | 28 | 123 | 291 | hsa-miR-801 | 2153 | 7 | 8.67e−016 |
| 2631 | 28 | 182 | 304, 317 | hsa-miR-92 | 2248 | 20 | 0 |
| 2632 | 28 | 6 | 471 | hsa-miR-93 | 1523 | 20 | 0 |
| 2633 | 28 | 14 | 318 | hsa-miR-99a | 675 | 20 | 0 |
| 2634 | 29 | 196 | 508 | 15_rel | 41856 | 6 | 1.61e−016 |
| 2635 | 29 | 134 | 436 | 24_5_rel | 43595 | 3 | 6.58e−007 |
| 2636 | 29 | 197 | 444 | 25_6 | 534 | 4 | 1.98e−010 |
| 2637 | 29 | 219 | 497 | 27_3 | 1041 | 4 | 4.59e−009 |
| 2638 | 29 | 79 | 460 | 43_37_rel | 28331 | 3 | 2.37e−007 |
| 2639 | 29 | 129 | 548 | 43_39 | 569 | 4 | 1.76e−009 |
| 2640 | 29 | 145 | 447 | 43_44_rel | 49543 | 4 | 3.53e−009 |
| 2641 | 29 | 94 | 394 | 69_6_rel | 10969 | 6 | 4.34e−014 |
| 2642 | 29 | 177 | 366, 464 | 72_12 | 1444 | 6 | 1.72e−015 |
| 2643 | 29 | 40 | 553 | 72_4 | 1691 | 6 | 2.78e−013 |
| 2644 | 29 | 97 | 357 | 74_26_rel | 1528 | 6 | 2.02e−013 |
| 2645 | 29 | 33 | 339, 552 | 96_rel | 26545 | 6 | 6.72e−015 |
| 2646 | 29 | 87 | 301 | gam11 | 15681 | 6 | 3.76e−015 |
| 2647 | 29 | 223 | 320, 393, 559 | hsa-let-7a | 2814 | 6 | 1.63e−013 |
| 2648 | 29 | 223 | 320, 393, 559 | hsa-let-7a | 1491 | 3 | 3.36e−006 |
| 2649 | 29 | 225 | 332 | hsa-let-7b | 13543 | 6 | 2.4e−015 |
| 2650 | 29 | 224 | 427 | hsa-let-7c | 5605 | 6 | 1.87e−014 |
| 2651 | 29 | 45 | 277 | hsa-let-7d | 1243 | 6 | 4e−014 |
| 2652 | 29 | 220 | 475 | hsa-let-7e | 1505 | 6 | 1.11e−014 |
| 2653 | 29 | 226 | 395 | hsa-let-7g | 729 | 6 | 4.82e−015 |
| 2654 | 29 | 227 | 297 | hsa-let-7i | 1187 | 6 | 3.16e−016 |
| 2655 | 29 | 13 | 397 | hsa-miR-100 | 1192 | 6 | 1.28e−014 |
| 2656 | 29 | 48 | 483, 529 | hsa-miR-103 | 1704 | 6 | 8.84e−015 |
| 2657 | 29 | 2 | 399 | hsa-miR-106a | 763 | 6 | 4.28e−014 |
| 2658 | 29 | 149 | 392 | hsa-miR-106b | 702 | 6 | 4.1e−016 |
| 2659 | 29 | 47 | 555 | hsa-miR-107 | 1543 | 6 | 1.78e−014 |
| 2660 | 29 | 199 | 387 | hsa-miR-125a | 2839 | 6 | 5.58e−015 |
| 2661 | 29 | 198 | 313, 551 | hsa-miR-125b | 5768 | 6 | 1.36e−014 |
| 2662 | 29 | 204 | 419 | hsa-miR-126 | 1679 | 6 | 8.12e−013 |
| 2663 | 29 | 89 | 419 | hsa-miR-126* | 630 | 6 | 2.34e−014 |
| 2664 | 29 | 86 | 417 | hsa-miR-130a | 963 | 6 | 9.98e−014 |
| 2665 | 29 | 151 | 472 | hsa-miR-141 | 2409 | 6 | 3.5e−014 |
| 2666 | 29 | 218 | 527 | hsa-miR-143 | 3660 | 6 | 2.62e−014 |
| 2667 | 29 | 139 | 281 | hsa-miR-145 | 4749 | 6 | 5.39e−015 |
| 2668 | 29 | 209 | 507 | hsa-miR-150 | 673 | 6 | 1.48e−014 |
| 2669 | 29 | 172 | 329 | hsa-miR-15b | 623 | 6 | 4.44e−015 |
| 2670 | 29 | 173 | 360, 468 | hsa-miR-16 | 2134 | 6 | 4.1e−013 |
| 2671 | 29 | 75 | 467 | hsa-miR-17-5p | 692 | 6 | 1.35e−014 |
| 2672 | 29 | 10 | 371, 461 | hsa-miR-181a | 2757 | 6 | 3e−014 |
| 2673 | 29 | 11 | 456, 540 | hsa-miR-181b | 745 | 6 | 1.05e−014 |
| 2674 | 29 | 77 | 440 | hsa-miR-191 | 1301 | 6 | 1.42e−014 |
| 2675 | 29 | 130 | 440 | hsa-miR-191* | 815 | 6 | 1.84e−013 |
| 2676 | 29 | 17 | 442 | hsa-miR-193a | 1477 | 6 | 4.02e−015 |
| 2677 | 29 | 16 | 488 | hsa-miR-193b | 798 | 6 | 3.38e−014 |
| 2678 | 29 | 170 | 327 | hsa-miR-195 | 568 | 6 | 4.7e−014 |
| 2679 | 29 | 92 | 410, 432 | hsa-miR-199a | 3634 | 6 | 1.53e−015 |
| 2680 | 29 | 162 | 410, 432, 473 | hsa-miR-199a* | 724 | 6 | 4.72e−015 |
| 2681 | 29 | 251 | 282, 495 | hsa-miR-19b | 514 | 6 | 1.39e−015 |
| 2682 | 29 | 152 | 369 | hsa-miR-200a | 1002 | 6 | 9.69e−015 |
| 2683 | 29 | 157 | 411 | hsa-miR-200b | 1296 | 6 | 1.34e−014 |
| 2684 | 29 | 156 | 446 | hsa-miR-200c | 3521 | 6 | 6.02e−014 |
| 2685 | 29 | 203 | 504 | hsa-miR-205 | 1553 | 6 | 1.97e−011 |
| 2686 | 29 | 175 | 462 | hsa-miR-21 | 6490 | 6 | 1.67e−013 |
| 2687 | 29 | 115 | 314 | hsa-miR-210 | 832 | 6 | 1.24e−013 |
| 2688 | 29 | 29 | 439 | hsa-miR-214 | 1558 | 6 | 8.64e−014 |
| 2689 | 29 | 18 | 322 | hsa-miR-22 | 3100 | 6 | 9.54e−015 |
| 2690 | 29 | 50 | 364 | hsa-miR-221 | 2736 | 6 | 2.01e−016 |
| 2691 | 29 | 49 | 302 | hsa-miR-222 | 2696 | 6 | 2.02e−015 |
| 2692 | 29 | 67 | 336 | hsa-miR-23a | 4535 | 6 | 1.97e−014 |
| 2693 | 29 | 66 | 306 | hsa-miR-23b | 963 | 6 | 7.75e−011 |
| 2694 | 29 | 238 | 346, 416 | hsa-miR-24 | 7913 | 6 | 7.69e−015 |
| 2695 | 29 | 90 | 388 | hsa-miR-25 | 542 | 6 | 1.53e−015 |
| 2696 | 29 | 256 | 441, 487 | hsa-miR-26a | 4952 | 6 | 2.96e−013 |
| 2697 | 29 | 259 | 481 | hsa-miR-27a | 2861 | 6 | 4.58e−014 |
| 2698 | 29 | 260 | 280 | hsa-miR-27b | 833 | 6 | 1.97e−012 |
| 2699 | 29 | 61 | 286 | hsa-miR-296 | 1295 | 6 | 4.86e−014 |
| 2700 | 29 | 167 | 556 | hsa-miR-29a | 1759 | 6 | 4.26e−014 |
| 2701 | 29 | 244 | 287 | hsa-miR-30a-5p | 1272 | 6 | 1.76e−014 |
| 2702 | 29 | 242 | 311 | hsa-miR-30b | 575 | 6 | 1.3e−012 |
| 2703 | 29 | 243 | 299, 316 | hsa-miR-30c | 962 | 6 | 4.65e−014 |
| 2704 | 29 | 241 | 550 | hsa-miR-30d | 2356 | 6 | 4.13e−014 |
| 2705 | 29 | 245 | 431 | hsa-miR-30e-5p | 1083 | 6 | 3.99e−015 |
| 2706 | 29 | 132 | 429 | hsa-miR-31 | 1099 | 6 | 3.04e−013 |
| 2707 | 29 | 1 | 359 | hsa-miR-320 | 2995 | 6 | 1.58e−016 |
| 2708 | 29 | 113 | 538 | hsa-miR-328 | 555 | 6 | 3.09e−015 |
| 2709 | 29 | 141 | 437 | hsa-miR-33 | 692 | 6 | 1.65e−012 |
| 2710 | 29 | 126 | 380 | hsa-miR-331 | 778 | 6 | 1.3e−015 |
| 2711 | 29 | 194 | 337 | hsa-miR-338 | 612 | 6 | 3.01e−012 |
| 2712 | 29 | 208 | 477 | hsa-miR-342 | 703 | 6 | 6.48e−015 |
| 2713 | 29 | 237 | 480 | hsa-miR-34a | 1346 | 6 | 1.61e−014 |
| 2714 | 29 | 57 | 498 | hsa-miR-34c | 530 | 6 | 2.37e−013 |
| 2715 | 29 | 128 | 294 | hsa-miR-370 | 701 | 6 | 2.11e−014 |
| 2716 | 29 | 112 | 372 | hsa-miR-422b | 779 | 6 | 3.68e−012 |
| 2717 | 29 | 51 | 497 | hsa-miR-423 | 1504 | 6 | 2.29e−015 |
| 2718 | 29 | 236 | 413 | hsa-miR-449 | 730 | 6 | 2.71e−013 |
| 2719 | 29 | 44 | 547 | hsa-miR-485-5p | 567 | 6 | 3.09e−014 |
| 2720 | 29 | 63 | 557 | hsa-miR-491 | 1439 | 6 | 3.26e−014 |
| 2721 | 29 | 214 | 376 | hsa-miR-494 | 11505 | 6 | 1.03e−013 |
| 2722 | 29 | 83 | 312 | hsa-miR-497 | 905 | 6 | 1.06e−015 |
| 2723 | 29 | 258 | 428, 443 | hsa-miR-513 | 671 | 6 | 1.05e−013 |
| 2724 | 29 | 102 | 344 | hsa-miR-520a* | 507 | 6 | 7.68e−014 |
| 2725 | 29 | 103 | 342 | hsa-miR-525 | 589 | 6 | 1.21e−013 |
| 2726 | 29 | 108 | 501 | hsa-miR-526b | 511 | 6 | 2.82e−013 |
| 2727 | 29 | 135 | 315 | hsa-miR-565 | 1619 | 6 | 6.39e−014 |
| 2728 | 29 | 60 | 479 | hsa-miR-638 | 7824 | 6 | 2.22e−014 |
| 2729 | 29 | 59 | 330 | hsa-miR-663 | 538 | 6 | 3.67e−012 |
| 2730 | 29 | 186 | 341 | hsa-miR-768-3p | 2194 | 5 | 2.32e−012 |
| 2731 | 29 | 147 | 341 | hsa-miR-768-5p | 541 | 3 | 1.01e−006 |
| 2732 | 29 | 123 | 291 | hsa-miR-801 | 1585 | 5 | 2.75e−011 |
| 2733 | 29 | 182 | 304, 317 | hsa-miR-92 | 2021 | 6 | 2.38e−014 |
| 2734 | 29 | 6 | 471 | hsa-miR-93 | 1062 | 6 | 8.13e−015 |
| 2735 | 29 | 14 | 318 | hsa-miR-99a | 1158 | 6 | 7.65e−015 |
| 2736 | 29 | 80 | 509 | hsa-miR-99b | 599 | 6 | 6.94e−015 |
| 2737 | 30 | 196 | 508 | 15_rel | 36931 | 11 | 2.54e−031 |
| 2738 | 30 | 134 | 436 | 24_5_rel | 36177 | 7 | 1.29e−019 |
| 2739 | 30 | 219 | 497 | 27_3 | 884 | 4 | 5.65e−009 |
| 2740 | 30 | 79 | 460 | 43_37_rel | 20037 | 7 | 1.86e−020 |
| 2741 | 30 | 145 | 447 | 43_44_rel | 33745 | 3 | 1.56e−006 |
| 2742 | 30 | 94 | 394 | 69_6_rel | 4933 | 11 | 2.29e−028 |
| 2743 | 30 | 40 | 553 | 72_4 | 681 | 11 | 3.33e−026 |
| 2744 | 30 | 97 | 357 | 74_26_rel | 1006 | 11 | 3.39e−028 |
| 2745 | 30 | 33 | 339, 552 | 96_rel | 11485 | 10 | 1.69e−025 |
| 2746 | 30 | 87 | 301 | gam11 | 8004 | 11 | 1.83e−029 |
| 2747 | 30 | 223 | 320, 393, 559 | hsa-let-7a | 734 | 11 | 4.96e−026 |
| 2748 | 30 | 225 | 332 | hsa-let-7b | 6430 | 11 | 3.89e−030 |
| 2749 | 30 | 224 | 427 | hsa-let-7c | 1731 | 11 | 4.04e−028 |
| 2750 | 30 | 45 | 277 | hsa-let-7d | 783 | 11 | 7.31e−029 |
| 2751 | 30 | 220 | 475 | hsa-let-7e | 970 | 11 | 1.87e−032 |
| 2752 | 30 | 226 | 395 | hsa-let-7g | 720 | 11 | 1e−029 |
| 2753 | 30 | 227 | 297 | hsa-let-7i | 769 | 11 | 6.49e−029 |
| 2754 | 30 | 13 | 397 | hsa-miR-100 | 896 | 11 | 1.41e−028 |
| 2755 | 30 | 48 | 483, 529 | hsa-miR-103 | 869 | 11 | 1.78e−028 |
| 2756 | 30 | 149 | 392 | hsa-miR-106b | 680 | 11 | 7.04e−029 |
| 2757 | 30 | 47 | 555 | hsa-miR-107 | 806 | 11 | 1.11e−028 |
| 2758 | 30 | 199 | 387 | hsa-miR-125a | 963 | 11 | 6.32e−031 |
| 2759 | 30 | 198 | 313, 551 | hsa-miR-125b | 2127 | 11 | 1.6e−030 |
| 2760 | 30 | 204 | 419 | hsa-miR-126 | 781 | 11 | 1.14e−028 |
| 2761 | 30 | 89 | 419 | hsa-miR-126* | 678 | 11 | 2.98e−027 |
| 2762 | 30 | 153 | 445 | hsa-miR-132 | 524 | 11 | 4.79e−027 |
| 2763 | 30 | 151 | 472 | hsa-miR-141 | 1959 | 11 | 7.48e−029 |
| 2764 | 30 | 218 | 527 | hsa-miR-143 | 1265 | 11 | 8.13e−025 |
| 2765 | 30 | 139 | 281 | hsa-miR-145 | 2346 | 11 | 8.07e−030 |
| 2766 | 30 | 209 | 507 | hsa-miR-150 | 670 | 11 | 4.32e−029 |
| 2767 | 30 | 173 | 360, 468 | hsa-miR-16 | 725 | 11 | 2.07e−026 |
| 2768 | 30 | 10 | 371, 461 | hsa-miR-181a | 1640 | 11 | 8.16e−029 |
| 2769 | 30 | 77 | 440 | hsa-miR-191 | 1103 | 11 | 8.45e−031 |
| 2770 | 30 | 130 | 440 | hsa-miR-191* | 631 | 11 | 4.03e−028 |
| 2771 | 30 | 17 | 442 | hsa-miR-193a | 623 | 11 | 9.74e−029 |
| 2772 | 30 | 92 | 410, 432 | hsa-miR-199a | 1303 | 11 | 1.12e−029 |
| 2773 | 30 | 152 | 369 | hsa-miR-200a | 542 | 11 | 6.99e−026 |
| 2774 | 30 | 157 | 411 | hsa-miR-200b | 603 | 11 | 2.66e−028 |
| 2775 | 30 | 156 | 446 | hsa-miR-200c | 2676 | 11 | 3.11e−030 |
| 2776 | 30 | 203 | 504 | hsa-miR-205 | 1543 | 11 | 4.04e−023 |
| 2777 | 30 | 175 | 462 | hsa-miR-21 | 2494 | 11 | 6.57e−025 |
| 2778 | 30 | 115 | 314 | hsa-miR-210 | 531 | 11 | 1.17e−029 |
| 2779 | 30 | 29 | 439 | hsa-miR-214 | 629 | 11 | 1.99e−029 |
| 2780 | 30 | 18 | 322 | hsa-miR-22 | 1421 | 11 | 3.73e−028 |
| 2781 | 30 | 50 | 364 | hsa-miR-221 | 1150 | 11 | 2.23e−027 |
| 2782 | 30 | 49 | 302 | hsa-miR-222 | 1386 | 11 | 1.59e−028 |
| 2783 | 30 | 67 | 336 | hsa-miR-23a | 1476 | 11 | 5.77e−028 |
| 2784 | 30 | 66 | 306 | hsa-miR-23b | 1104 | 11 | 1.71e−024 |
| 2785 | 30 | 238 | 346, 416 | hsa-miR-24 | 3395 | 11 | 1.44e−027 |
| 2786 | 30 | 90 | 388 | hsa-miR-25 | 626 | 11 | 2.37e−031 |
| 2787 | 30 | 256 | 441, 487 | hsa-miR-26a | 1604 | 11 | 1.13e−026 |
| 2788 | 30 | 259 | 481 | hsa-miR-27a | 1451 | 11 | 3.75e−027 |
| 2789 | 30 | 61 | 286 | hsa-miR-296 | 557 | 11 | 1.08e−025 |
| 2790 | 30 | 244 | 287 | hsa-miR-30a-5p | 800 | 11 | 8.99e−026 |
| 2791 | 30 | 241 | 550 | hsa-miR-30d | 1173 | 11 | 9.73e−029 |
| 2792 | 30 | 245 | 431 | hsa-miR-30e-5p | 615 | 11 | 4.37e−028 |
| 2793 | 30 | 132 | 429 | hsa-miR-31 | 848 | 11 | 6.24e−028 |
| 2794 | 30 | 1 | 359 | hsa-miR-320 | 1462 | 11 | 4.69e−032 |
| 2795 | 30 | 91 | 368 | hsa-miR-324-3p | 574 | 11 | 3.45e−028 |
| 2796 | 30 | 113 | 538 | hsa-miR-328 | 578 | 11 | 1.61e−028 |
| 2797 | 30 | 126 | 380 | hsa-miR-331 | 520 | 11 | 4.42e−030 |
| 2798 | 30 | 237 | 480 | hsa-miR-34a | 1027 | 11 | 4.48e−027 |
| 2799 | 30 | 128 | 294 | hsa-miR-370 | 681 | 11 | 5.85e−027 |
| 2800 | 30 | 111 | 389 | hsa-miR-422a | 776 | 11 | 6.42e−026 |
| 2801 | 30 | 112 | 372 | hsa-miR-422b | 996 | 11 | 9.44e−027 |
| 2802 | 30 | 51 | 497 | hsa-miR-423 | 1020 | 11 | 1.87e−031 |
| 2803 | 30 | 236 | 413 | hsa-miR-449 | 531 | 11 | 2.41e−027 |
| 2804 | 30 | 44 | 547 | hsa-miR-485-5p | 561 | 11 | 1.48e−028 |
| 2805 | 30 | 63 | 557 | hsa-miR-491 | 1007 | 11 | 1.66e−027 |
| 2806 | 30 | 214 | 376 | hsa-miR-494 | 2590 | 11 | 9.91e−028 |
| 2807 | 30 | 258 | 428, 443 | hsa-miR-513 | 607 | 11 | 6.64e−028 |
| 2808 | 30 | 120 | 522 | hsa-miR-515-3p | 554 | 11 | 1.05e−025 |
| 2809 | 30 | 135 | 315 | hsa-miR-565 | 1426 | 11 | 6.78e−028 |
| 2810 | 30 | 60 | 479 | hsa-miR-638 | 3885 | 11 | 3.2e−028 |
| 2811 | 30 | 186 | 341 | hsa-miR-768-3p | 1021 | 10 | 1.14e−024 |
| 2812 | 30 | 123 | 291 | hsa-miR-801 | 928 | 10 | 1.59e−026 |
| 2813 | 30 | 182 | 304, 317 | hsa-miR-92 | 1339 | 11 | 2.08e−029 |
| 2814 | 30 | 6 | 471 | hsa-miR-93 | 927 | 11 | 1.44e−028 |
| 2815 | 30 | 14 | 318 | hsa-miR-99a | 795 | 11 | 4.82e−031 |
| 2816 | 30 | 80 | 509 | hsa-miR-99b | 528 | 11 | 2.49e−030 |
| 2817 | 31 | 196 | 508 | 15_rel | 45806 | 6 | 1.07e−015 |
| 2818 | 31 | 197 | 444 | 25_6 | 1027 | 6 | 3.91e−015 |
| 2819 | 31 | 219 | 497 | 27_3 | 1765 | 6 | 1.8e−015 |
| 2820 | 31 | 7 | 337 | 43_21 | 602 | 6 | 1.7e−015 |
| 2821 | 31 | 42 | 449 | 43_3 | 679 | 6 | 9.67e−016 |
| 2822 | 31 | 101 | 475 | 43_30 | 839 | 6 | 8.18e−016 |
| 2823 | 31 | 99 | 348, 384, 434 | 43_35 | 647 | 6 | 3.11e−015 |
| 2824 | 31 | 129 | 548 | 43_39 | 2257 | 6 | 6.26e−016 |
| 2825 | 31 | 27 | 313 | 43_40 | 528 | 6 | 1.6e−015 |
| 2826 | 31 | 145 | 447 | 43_44_rel | 51547 | 6 | 1.33e−014 |
| 2827 | 31 | 114 | 530 | 43_6 | 579 | 6 | 9.33e−017 |
| 2828 | 31 | 183 | 373 | 66_1 | 933 | 6 | 1.43e−015 |
| 2829 | 31 | 94 | 394 | 69_6_rel | 20072 | 6 | 1.27e−016 |
| 2830 | 31 | 177 | 366, 464 | 72_12 | 1784 | 6 | 2.29e−013 |
| 2831 | 31 | 40 | 553 | 72_4 | 677 | 6 | 3.98e−014 |
| 2832 | 31 | 97 | 357 | 74_26_rel | 5457 | 6 | 9.49e−015 |
| 2833 | 31 | 33 | 339, 552 | 96_rel | 31259 | 6 | 5.52e−016 |
| 2834 | 31 | 87 | 301 | gam11 | 24373 | 6 | 1.57e−014 |
| 2835 | 31 | 125 | 532 | 70_16 | 541 | 6 | 2.3e−014 |
| 2836 | 31 | 163 | 402 | 43_7 | 947 | 6 | 3.24e−014 |
| 2837 | 31 | 223 | 320, 393, 559 | hsa-let-7a | 4878 | 6 | 5.77e−013 |
| 2838 | 31 | 223 | 320, 393, 559 | hsa-let-7a | 1669 | 6 | 8.43e−012 |
| 2839 | 31 | 225 | 332 | hsa-let-7b | 4284 | 6 | 3.06e−011 |
| 2840 | 31 | 224 | 427 | hsa-let-7c | 5908 | 6 | 3.91e−015 |
| 2841 | 31 | 45 | 277 | hsa-let-7d | 1326 | 6 | 4.93e−012 |
| 2842 | 31 | 220 | 475 | hsa-let-7e | 2781 | 6 | 8.82e−016 |
| 2843 | 31 | 222 | 482, 558 | hsa-let-7f | 585 | 6 | 1.88e−015 |
| 2844 | 31 | 226 | 395 | hsa-let-7g | 1705 | 6 | 6.14e−015 |
| 2845 | 31 | 227 | 297 | hsa-let-7i | 2452 | 6 | 4.26e−015 |
| 2846 | 31 | 231 | 321, 424 | hsa-miR-1 | 544 | 6 | 1.65e−016 |
| 2847 | 31 | 13 | 397 | hsa-miR-100 | 1790 | 6 | 2.07e−013 |
| 2848 | 31 | 48 | 483, 529 | hsa-miR-103 | 2004 | 6 | 2.73e−014 |
| 2849 | 31 | 184 | 423 | hsa-miR-105 | 1111 | 6 | 2.87e−015 |
| 2850 | 31 | 2 | 399 | hsa-miR-106a | 2045 | 6 | 1e−012 |
| 2851 | 31 | 149 | 392 | hsa-miR-106b | 2088 | 6 | 5.45e−016 |
| 2852 | 31 | 47 | 555 | hsa-miR-107 | 1886 | 6 | 4.73e−015 |
| 2853 | 31 | 199 | 387 | hsa-miR-125a | 1505 | 6 | 2.24e−015 |
| 2854 | 31 | 198 | 313, 551 | hsa-miR-125b | 6320 | 6 | 1.05e−013 |
| 2855 | 31 | 204 | 419 | hsa-miR-126 | 899 | 6 | 5.91e−014 |
| 2856 | 31 | 89 | 419 | hsa-miR-126* | 1478 | 6 | 1.14e−013 |
| 2857 | 31 | 86 | 417 | hsa-miR-130a | 3014 | 6 | 2.45e−015 |
| 2858 | 31 | 85 | 328 | hsa-miR-130b | 727 | 6 | 1.54e−014 |
| 2859 | 31 | 153 | 445 | hsa-miR-132 | 527 | 6 | 1.21e−015 |
| 2860 | 31 | 266 | 295, 386 | hsa-miR-133a | 705 | 6 | 3.88e−015 |
| 2861 | 31 | 265 | 533 | hsa-miR-133b | 621 | 6 | 3.04e−014 |
| 2862 | 31 | 181 | 324, 554 | hsa-miR-135a | 690 | 6 | 1.63e−016 |
| 2863 | 31 | 37 | 391 | hsa-miR-136 | 799 | 6 | 8.17e−017 |
| 2864 | 31 | 52 | 370, 548 | hsa-miR-138 | 1054 | 6 | 6.44e−016 |
| 2865 | 31 | 64 | 402 | hsa-miR-140 | 481 | 6 | 1.39e−016 |
| 2866 | 31 | 151 | 472 | hsa-miR-141 | 577 | 6 | 4.64e−015 |
| 2867 | 31 | 247 | 278 | hsa-miR-142-3p | 707 | 6 | 3.01e−015 |
| 2868 | 31 | 218 | 527 | hsa-miR-143 | 2266 | 6 | 3.89e−015 |
| 2869 | 31 | 139 | 281 | hsa-miR-145 | 1574 | 6 | 3.78e−015 |
| 2870 | 31 | 217 | 466 | hsa-miR-146a | 1375 | 6 | 6.11e−012 |
| 2871 | 31 | 146 | 325 | hsa-miR-147 | 569 | 6 | 1.93e−015 |
| 2872 | 31 | 191 | 289 | hsa-miR-148b | 573 | 6 | 2.19e−016 |
| 2873 | 31 | 212 | 333 | hsa-miR-149 | 501 | 6 | 6.04e−013 |
| 2874 | 31 | 209 | 507 | hsa-miR-150 | 2371 | 6 | 2.06e−012 |
| 2875 | 31 | 192 | 470 | hsa-miR-152 | 534 | 6 | 1.11e−015 |
| 2876 | 31 | 171 | 331 | hsa-miR-15a | 564 | 6 | 1.04e−015 |
| 2877 | 31 | 172 | 329 | hsa-miR-15b | 884 | 6 | 1.17e−012 |
| 2878 | 31 | 173 | 360, 468 | hsa-miR-16 | 2568 | 6 | 1.62e−011 |
| 2879 | 31 | 38 | 467 | hsa-miR-17-3p | 611 | 6 | 1.44e−014 |
| 2880 | 31 | 75 | 467 | hsa-miR-17-5p | 1539 | 6 | 3.73e−013 |
| 2881 | 31 | 10 | 371, 461 | hsa-miR-181a | 3471 | 6 | 1.75e−014 |
| 2882 | 31 | 32 | 461 | hsa-miR-181a* | 528 | 6 | 6.98e−016 |
| 2883 | 31 | 11 | 456, 540 | hsa-miR-181b | 921 | 6 | 1.43e−014 |
| 2884 | 31 | 179 | 356 | hsa-miR-183 | 1590 | 6 | 2.74e−014 |
| 2885 | 31 | 233 | 305 | hsa-miR-184 | 615 | 6 | 5.05e−015 |
| 2886 | 31 | 234 | 450 | hsa-miR-185 | 724 | 6 | 5.26e−016 |
| 2887 | 31 | 88 | 310 | hsa-miR-188 | 891 | 6 | 1.34e−015 |
| 2888 | 31 | 144 | 346 | hsa-miR-189 | 721 | 6 | 7.9e−016 |
| 2889 | 31 | 77 | 440 | hsa-miR-191 | 1623 | 6 | 9.43e−016 |
| 2890 | 31 | 130 | 440 | hsa-miR-191* | 1106 | 6 | 9.52e−015 |
| 2891 | 31 | 17 | 442 | hsa-miR-193a | 1408 | 6 | 1.48e−015 |
| 2892 | 31 | 170 | 327 | hsa-miR-195 | 562 | 6 | 6.87e−013 |
| 2893 | 31 | 92 | 410, 432 | hsa-miR-199a | 1506 | 6 | 4.33e−014 |
| 2894 | 31 | 162 | 410, 432, 473 | hsa-miR-199a* | 596 | 6 | 1.67e−012 |
| 2895 | 31 | 93 | 473 | hsa-miR-199b | 549 | 6 | 2.22e−015 |
| 2896 | 31 | 251 | 282, 495 | hsa-miR-19b | 1434 | 6 | 1.43e−013 |
| 2897 | 31 | 152 | 369 | hsa-miR-200a | 508 | 6 | 4.02e−016 |
| 2898 | 31 | 157 | 411 | hsa-miR-200b | 602 | 6 | 1.27e−015 |
| 2899 | 31 | 156 | 446 | hsa-miR-200c | 666 | 6 | 3.38e−015 |
| 2900 | 31 | 271 | 334 | hsa-miR-202* | 807 | 6 | 1.69e−016 |
| 2901 | 31 | 232 | 422 | hsa-miR-206 | 586 | 6 | 2.85e−015 |
| 2902 | 31 | 175 | 462 | hsa-miR-21 | 5903 | 6 | 1.39e−013 |
| 2903 | 31 | 115 | 314 | hsa-miR-210 | 759 | 6 | 1.29e−015 |
| 2904 | 31 | 29 | 439 | hsa-miR-214 | 1372 | 6 | 2.18e−014 |
| 2905 | 31 | 159 | 494 | hsa-miR-216 | 985 | 6 | 1.49e−014 |
| 2906 | 31 | 268 | 401, 425 | hsa-miR-218 | 509 | 6 | 1.58e−015 |
| 2907 | 31 | 18 | 322 | hsa-miR-22 | 2462 | 6 | 2.45e−014 |
| 2908 | 31 | 50 | 364 | hsa-miR-221 | 2447 | 6 | 2.79e−014 |
| 2909 | 31 | 49 | 302 | hsa-miR-222 | 2783 | 6 | 1.15e−014 |
| 2910 | 31 | 248 | 415 | hsa-miR-223 | 756 | 6 | 1.78e−016 |
| 2911 | 31 | 67 | 336 | hsa-miR-23a | 2932 | 6 | 1.9e−012 |
| 2912 | 31 | 238 | 346, 416 | hsa-miR-24 | 6200 | 6 | 5.69e−014 |
| 2913 | 31 | 90 | 388 | hsa-miR-25 | 1342 | 6 | 2.24e−016 |
| 2914 | 31 | 256 | 441, 487 | hsa-miR-26a | 6139 | 6 | 2.15e−013 |
| 2915 | 31 | 257 | 409 | hsa-miR-26b | 846 | 6 | 1e−015 |
| 2916 | 31 | 259 | 481 | hsa-miR-27a | 4346 | 6 | 1.03e−012 |
| 2917 | 31 | 260 | 280 | hsa-miR-27b | 698 | 6 | 1.51e−012 |
| 2918 | 31 | 61 | 286 | hsa-miR-296 | 3897 | 6 | 1.4e−014 |
| 2919 | 31 | 167 | 556 | hsa-miR-29a | 2871 | 6 | 1.02e−014 |
| 2920 | 31 | 269 | 362 | hsa-miR-302c* | 648 | 6 | 1.14e−016 |
| 2921 | 31 | 117 | 287 | hsa-miR-30a-3p | 1027 | 6 | 3.43e−015 |
| 2922 | 31 | 244 | 287 | hsa-miR-30a-5p | 1745 | 6 | 1.72e−014 |
| 2923 | 31 | 242 | 311 | hsa-miR-30b | 1031 | 6 | 1.33e−014 |
| 2924 | 31 | 243 | 299, 316 | hsa-miR-30c | 1406 | 6 | 1.62e−013 |
| 2925 | 31 | 241 | 550 | hsa-miR-30d | 2171 | 6 | 8.23e−015 |
| 2926 | 31 | 116 | 431 | hsa-miR-30e-3p | 604 | 6 | 2.17e−015 |
| 2927 | 31 | 245 | 431 | hsa-miR-30e-5p | 2130 | 6 | 9e−016 |
| 2928 | 31 | 132 | 429 | hsa-miR-31 | 1434 | 6 | 3.3e−014 |
| 2929 | 31 | 1 | 359 | hsa-miR-320 | 5099 | 6 | 1.51e−014 |
| 2930 | 31 | 91 | 368 | hsa-miR-324-3p | 1289 | 6 | 1.13e−014 |
| 2931 | 31 | 113 | 538 | hsa-miR-328 | 1607 | 6 | 2.28e−015 |
| 2932 | 31 | 141 | 437 | hsa-miR-33 | 2168 | 6 | 6.34e−016 |
| 2933 | 31 | 126 | 380 | hsa-miR-331 | 1246 | 6 | 4.03e−017 |
| 2934 | 31 | 185 | 492 | hsa-miR-335 | 823 | 6 | 1.29e−018 |
| 2935 | 31 | 194 | 337 | hsa-miR-338 | 2690 | 6 | 6.77e−015 |
| 2936 | 31 | 208 | 477 | hsa-miR-342 | 1290 | 6 | 2.34e−014 |
| 2937 | 31 | 229 | 292 | hsa-miR-345 | 927 | 6 | 4.81e−016 |
| 2938 | 31 | 237 | 480 | hsa-miR-34a | 2884 | 6 | 2.24e−014 |
| 2939 | 31 | 255 | 444 | hsa-miR-361 | 832 | 6 | 2.82e−016 |
| 2940 | 31 | 160 | 379, 382 | hsa-miR-365 | 1093 | 6 | 6e−015 |
| 2941 | 31 | 128 | 294 | hsa-miR-370 | 1321 | 6 | 1.39e−015 |
| 2942 | 31 | 35 | 406 | hsa-miR-373* | 1420 | 6 | 8.08e−015 |
| 2943 | 31 | 254 | 496 | hsa-miR-374 | 652 | 6 | 1.22e−016 |
| 2944 | 31 | 105 | 372 | hsa-miR-378 | 529 | 6 | 8.99e−017 |
| 2945 | 31 | 239 | 349 | hsa-miR-379 | 535 | 6 | 3.41e−015 |
| 2946 | 31 | 178 | 352 | hsa-miR-381 | 554 | 6 | 2.06e−015 |
| 2947 | 31 | 46 | 298 | hsa-miR-383 | 527 | 6 | 4.85e−016 |
| 2948 | 31 | 111 | 389 | hsa-miR-422a | 637 | 6 | 2.06e−015 |
| 2949 | 31 | 112 | 372 | hsa-miR-422b | 1045 | 6 | 1.3e−014 |
| 2950 | 31 | 51 | 497 | hsa-miR-423 | 2987 | 6 | 4.02e−015 |
| 2951 | 31 | 69 | 363 | hsa-miR-425-3p | 671 | 6 | 2.84e−014 |
| 2952 | 31 | 236 | 413 | hsa-miR-449 | 827 | 6 | 5.9e−011 |
| 2953 | 31 | 274 | 375, 309 | hsa-miR-450 | 507 | 6 | 1.34e−015 |
| 2954 | 31 | 252 | 405 | hsa-miR-452 | 775 | 6 | 6.14e−015 |
| 2955 | 31 | 189 | 500 | hsa-miR-484 | 529 | 6 | 6.89e−015 |
| 2956 | 31 | 44 | 547 | hsa-miR-485-5p | 1036 | 6 | 1.39e−014 |
| 2957 | 31 | 62 | 485 | hsa-miR-489 | 663 | 6 | 1.63e−016 |
| 2958 | 31 | 63 | 557 | hsa-miR-491 | 4301 | 6 | 1.54e−015 |
| 2959 | 31 | 54 | 293 | hsa-miR-492 | 1047 | 6 | 8.88e−016 |
| 2960 | 31 | 267 | 354 | hsa-miR-493-5p | 747 | 6 | 1.43e−015 |
| 2961 | 31 | 214 | 376 | hsa-miR-494 | 11329 | 6 | 1.7e−015 |
| 2962 | 31 | 3 | 543 | hsa-miR-495 | 716 | 6 | 3.81e−016 |
| 2963 | 31 | 83 | 312 | hsa-miR-497 | 764 | 6 | 1.68e−013 |
| 2964 | 31 | 270 | 390 | hsa-miR-498 | 830 | 6 | 7.38e−015 |
| 2965 | 31 | 71 | 407 | hsa-miR-500 | 537 | 6 | 1.52e−015 |
| 2966 | 31 | 25 | 414 | hsa-miR-501 | 555 | 6 | 1.05e−016 |
| 2967 | 31 | 68 | 288 | hsa-miR-502 | 588 | 6 | 1.65e−015 |
| 2968 | 31 | 174 | 534 | hsa-miR-503 | 644 | 6 | 6.87e−016 |
| 2969 | 31 | 43 | 418 | hsa-miR-504 | 1196 | 6 | 6.16e−016 |
| 2970 | 31 | 137 | 542 | hsa-miR-505 | 602 | 6 | 8.53e−017 |
| 2971 | 31 | 258 | 428, 443 | hsa-miR-513 | 1781 | 6 | 3.35e−015 |
| 2972 | 31 | 96 | 365, 478, 516 | hsa-miR-517* | 507 | 6 | 5.08e−016 |
| 2973 | 31 | 73 | 503 | hsa-miR-518b | 920 | 6 | 2.05e−015 |
| 2974 | 31 | 210 | 400 | hsa-miR-518c* | 982 | 6 | 1.56e−015 |
| 2975 | 31 | 106 | 525 | hsa-miR-518f* | 508 | 6 | 1.45e−016 |
| 2976 | 31 | 262 | 523 | hsa-miR-519e* | 755 | 6 | 3.77e−016 |
| 2977 | 31 | 102 | 344 | hsa-miR-520a* | 714 | 6 | 6.87e−016 |
| 2978 | 31 | 206 | 513 | hsa-miR-520d* | 966 | 6 | 2.11e−016 |
| 2979 | 31 | 100 | 524 | hsa-miR-524* | 716 | 6 | 2.02e−016 |
| 2980 | 31 | 103 | 342 | hsa-miR-525 | 907 | 6 | 9.09e−016 |
| 2981 | 31 | 118 | 342 | hsa-miR-525* | 539 | 6 | 3.48e−015 |
| 2982 | 31 | 108 | 501 | hsa-miR-526b | 840 | 6 | 1.29e−015 |
| 2983 | 31 | 110 | 511, 512, 514 | hsa-miR-527 | 771 | 6 | 8.09e−016 |
| 2984 | 31 | 148 | 283 | hsa-miR-557 | 623 | 6 | 1.74e−015 |
| 2985 | 31 | 135 | 315 | hsa-miR-565 | 2579 | 6 | 1.02e−014 |
| 2986 | 31 | 60 | 479 | hsa-miR-638 | 18596 | 6 | 5.48e−017 |
| 2987 | 31 | 195 | 421 | hsa-miR-662 | 2240 | 6 | 7.94e−016 |
| 2988 | 31 | 59 | 330 | hsa-miR-663 | 6968 | 6 | 1.69e−016 |
| 2989 | 31 | 36 | 385 | hsa-miR-766 | 972 | 6 | 3.65e−014 |
| 2990 | 31 | 186 | 341 | hsa-miR-768-3p | 3612 | 6 | 1.77e−013 |
| 2991 | 31 | 123 | 291 | hsa-miR-801 | 1771 | 6 | 2.06e−013 |
| 2992 | 31 | 182 | 304, 317 | hsa-miR-92 | 2501 | 6 | 5.71e−014 |
| 2993 | 31 | 6 | 471 | hsa-miR-93 | 2262 | 6 | 3.88e−015 |
| 2994 | 31 | 14 | 318 | hsa-miR-99a | 729 | 6 | 7.83e−014 |
| 2995 | 31 | 80 | 509 | hsa-miR-99b | 851 | 6 | 2.41e−015 |
| 2996 | 31 | 131 | 430 | 6_11 | 546 | 6 | 1.4e−014 |
| 2997 | 32 | 127 | 361 | 11_1 | 710 | 5 | 2.55e−009 |
| 2998 | 32 | 196 | 508 | 15_rel | 38523 | 5 | 1.02e−009 |
| 2999 | 32 | 20 | 452 | 24_3 | 611 | 5 | 4.63e−010 |
| 3000 | 32 | 94 | 394 | 69_6_rel | 754 | 5 | 2.74e−012 |
| 3001 | 32 | 177 | 366, 464 | 72_12 | 2222 | 5 | 7.9e−009 |
| 3002 | 32 | 97 | 357 | 74_26_rel | 794 | 5 | 9.51e−009 |
| 3003 | 32 | 87 | 301 | gam11 | 4379 | 5 | 1.45e−008 |
| 3004 | 32 | 163 | 402 | 43_7 | 710 | 5 | 3.46e−009 |
| 3005 | 32 | 223 | 320, 393, 559 | hsa-let-7a | 16401 | 5 | 7.93e−009 |
| 3006 | 32 | 225 | 332 | hsa-let-7b | 36719 | 5 | 4.33e−008 |
| 3007 | 32 | 224 | 427 | hsa-let-7c | 25152 | 5 | 7.77e−009 |
| 3008 | 32 | 45 | 277 | hsa-let-7d | 3926 | 5 | 1.04e−009 |
| 3009 | 32 | 220 | 475 | hsa-let-7e | 1443 | 5 | 2.68e−009 |
| 3010 | 32 | 222 | 482, 558 | hsa-let-7f | 619 | 5 | 6.97e−009 |
| 3011 | 32 | 227 | 297 | hsa-let-7i | 1269 | 5 | 1.87e−009 |
| 3012 | 32 | 13 | 397 | hsa-miR-100 | 6005 | 5 | 1.88e−008 |
| 3013 | 32 | 48 | 483, 529 | hsa-miR-103 | 1669 | 5 | 1.91e−008 |
| 3014 | 32 | 184 | 423 | hsa-miR-105 | 582 | 5 | 3.82e−009 |
| 3015 | 32 | 2 | 399 | hsa-miR-106a | 553 | 5 | 4.34e−009 |
| 3016 | 32 | 47 | 555 | hsa-miR-107 | 1538 | 5 | 7.15e−008 |
| 3017 | 32 | 165 | 340 | hsa-miR-10b | 962 | 5 | 6.25e−011 |
| 3018 | 32 | 199 | 387 | hsa-miR-125a | 6570 | 5 | 9.7e−010 |
| 3019 | 32 | 198 | 313, 551 | hsa-miR-125b | 50264 | 5 | 2.89e−010 |
| 3020 | 32 | 204 | 419 | hsa-miR-126 | 601 | 5 | 9.3e−008 |
| 3021 | 32 | 86 | 417 | hsa-miR-130a | 2724 | 5 | 4.01e−009 |
| 3022 | 32 | 218 | 527 | hsa-miR-143 | 8894 | 5 | 1.64e−007 |
| 3023 | 32 | 139 | 281 | hsa-miR-145 | 15932 | 5 | 9.6e−010 |
| 3024 | 32 | 212 | 333 | hsa-miR-149 | 838 | 5 | 3.11e−009 |
| 3025 | 32 | 172 | 329 | hsa-miR-15b | 579 | 5 | 8.05e−011 |
| 3026 | 32 | 173 | 360, 468 | hsa-miR-16 | 1249 | 5 | 2.46e−009 |
| 3027 | 32 | 234 | 450 | hsa-miR-185 | 609 | 5 | 3.21e−010 |
| 3028 | 32 | 77 | 440 | hsa-miR-191 | 989 | 5 | 6.13e−009 |
| 3029 | 32 | 17 | 442 | hsa-miR-193a | 4687 | 5 | 1.73e−009 |
| 3030 | 32 | 16 | 488 | hsa-miR-193b | 828 | 5 | 1.4e−010 |
| 3031 | 32 | 170 | 327 | hsa-miR-195 | 3825 | 5 | 3.65e−009 |
| 3032 | 32 | 92 | 410, 432 | hsa-miR-199a | 9750 | 5 | 1.67e−009 |
| 3033 | 32 | 162 | 410, 432, 473 | hsa-miR-199a* | 3540 | 5 | 1.8e−008 |
| 3034 | 32 | 251 | 282, 495 | hsa-miR-19b | 650 | 5 | 5.08e−009 |
| 3035 | 32 | 175 | 462 | hsa-miR-21 | 1585 | 5 | 1.28e−009 |
| 3036 | 32 | 29 | 439 | hsa-miR-214 | 1646 | 5 | 7.44e−008 |
| 3037 | 32 | 18 | 322 | hsa-miR-22 | 626 | 5 | 1.38e−009 |
| 3038 | 32 | 50 | 364 | hsa-miR-221 | 1691 | 5 | 8.5e−009 |
| 3039 | 32 | 49 | 302 | hsa-miR-222 | 3698 | 5 | 1.23e−008 |
| 3040 | 32 | 67 | 336 | hsa-miR-23a | 2415 | 5 | 1.63e−009 |
| 3041 | 32 | 66 | 306 | hsa-miR-23b | 5556 | 5 | 5.44e−009 |
| 3042 | 32 | 238 | 346, 416 | hsa-miR-24 | 5226 | 5 | 2.04e−009 |
| 3043 | 32 | 256 | 441, 487 | hsa-miR-26a | 18710 | 5 | 1e−009 |
| 3044 | 32 | 259 | 481 | hsa-miR-27a | 1906 | 5 | 3.84e−008 |
| 3045 | 32 | 260 | 280 | hsa-miR-27b | 1849 | 5 | 2.2e−008 |
| 3046 | 32 | 167 | 556 | hsa-miR-29a | 6411 | 5 | 4.51e−009 |
| 3047 | 32 | 242 | 311 | hsa-miR-30b | 1017 | 5 | 4.4e−009 |
| 3048 | 32 | 243 | 299, 316 | hsa-miR-30c | 1285 | 5 | 5.27e−009 |
| 3049 | 32 | 241 | 550 | hsa-miR-30d | 1354 | 5 | 8.41e−010 |
| 3050 | 32 | 245 | 431 | hsa-miR-30e-5p | 997 | 5 | 1.15e−008 |
| 3051 | 32 | 1 | 359 | hsa-miR-320 | 1073 | 5 | 7.93e−008 |
| 3052 | 32 | 113 | 538 | hsa-miR-328 | 540 | 5 | 1.61e−010 |
| 3053 | 32 | 141 | 437 | hsa-miR-33 | 734 | 5 | 2.65e−009 |
| 3054 | 32 | 126 | 380 | hsa-miR-331 | 880 | 5 | 7.9e−010 |
| 3055 | 32 | 194 | 337 | hsa-miR-338 | 1608 | 5 | 3.06e−009 |
| 3056 | 32 | 208 | 477 | hsa-miR-342 | 859 | 5 | 1.3e−009 |
| 3057 | 32 | 237 | 480 | hsa-miR-34a | 950 | 5 | 9.2e−010 |
| 3058 | 32 | 51 | 497 | hsa-miR-423 | 686 | 5 | 7.9e−010 |
| 3059 | 32 | 214 | 376 | hsa-miR-494 | 2780 | 5 | 1.05e−008 |
| 3060 | 32 | 83 | 312 | hsa-miR-497 | 3684 | 5 | 1.41e−010 |
| 3061 | 32 | 60 | 479 | hsa-miR-638 | 1317 | 5 | 1.46e−010 |
| 3062 | 32 | 182 | 304, 317 | hsa-miR-92 | 5179 | 5 | 1.75e−007 |
| 3063 | 32 | 14 | 318 | hsa-miR-99a | 20643 | 5 | 6.33e−010 |
| 3064 | 33 | 127 | 361 | 11_1 | 591 | 5 | 1.57e−008 |
| 3065 | 33 | 196 | 508 | 15_rel | 43666 | 5 | 5.68e−011 |
| 3066 | 33 | 94 | 394 | 69_6_rel | 1430 | 5 | 1.2e−008 |
| 3067 | 33 | 177 | 366, 464 | 72_12 | 719 | 5 | 5.14e−008 |
| 3068 | 33 | 97 | 357 | 74_26_rel | 721 | 5 | 5.26e−008 |
| 3069 | 33 | 87 | 301 | gam11 | 4134 | 5 | 3.22e−008 |
| 3070 | 33 | 223 | 320, 393, 559 | hsa-let-7a | 3300 | 5 | 6.64e−007 |
| 3071 | 33 | 225 | 332 | hsa-let-7b | 2998 | 5 | 9.22e−007 |
| 3072 | 33 | 224 | 427 | hsa-let-7c | 4206 | 5 | 2.23e−007 |
| 3073 | 33 | 45 | 277 | hsa-let-7d | 856 | 5 | 1.66e−008 |
| 3074 | 33 | 220 | 475 | hsa-let-7e | 1214 | 5 | 1.39e−009 |
| 3075 | 33 | 48 | 483, 529 | hsa-miR-103 | 1152 | 5 | 4.18e−007 |
| 3076 | 33 | 184 | 423 | hsa-miR-105 | 870 | 5 | 4.94e−008 |
| 3077 | 33 | 2 | 399 | hsa-miR-106a | 516 | 5 | 6.95e−008 |
| 3078 | 33 | 149 | 392 | hsa-miR-106b | 529 | 5 | 5.85e−009 |
| 3079 | 33 | 47 | 555 | hsa-miR-107 | 971 | 5 | 3.08e−007 |
| 3080 | 33 | 199 | 387 | hsa-miR-125a | 1145 | 5 | 2.87e−008 |
| 3081 | 33 | 198 | 313, 551 | hsa-miR-125b | 2541 | 5 | 5.82e−007 |
| 3082 | 33 | 86 | 417 | hsa-miR-130a | 965 | 5 | 5.74e−008 |
| 3083 | 33 | 151 | 472 | hsa-miR-141 | 571 | 5 | 3.37e−007 |
| 3084 | 33 | 218 | 527 | hsa-miR-143 | 890 | 5 | 1.42e−008 |
| 3085 | 33 | 139 | 281 | hsa-miR-145 | 872 | 5 | 4.2e−007 |
| 3086 | 33 | 173 | 360, 468 | hsa-miR-16 | 1003 | 5 | 1.38e−008 |
| 3087 | 33 | 75 | 467 | hsa-miR-17-5p | 518 | 5 | 8.93e−009 |
| 3088 | 33 | 17 | 442 | hsa-miR-193a | 887 | 5 | 1.44e−007 |
| 3089 | 33 | 92 | 410, 432 | hsa-miR-199a | 1190 | 5 | 2.76e−007 |
| 3090 | 33 | 152 | 369 | hsa-miR-200a | 579 | 5 | 6.93e−008 |
| 3091 | 33 | 156 | 446 | hsa-miR-200c | 2882 | 5 | 3.46e−009 |
| 3092 | 33 | 175 | 462 | hsa-miR-21 | 3694 | 5 | 7.08e−007 |
| 3093 | 33 | 50 | 364 | hsa-miR-221 | 860 | 5 | 3.08e−007 |
| 3094 | 33 | 49 | 302 | hsa-miR-222 | 2277 | 5 | 1.85e−008 |
| 3095 | 33 | 67 | 336 | hsa-miR-23a | 1203 | 5 | 2.52e−009 |
| 3096 | 33 | 66 | 306 | hsa-miR-23b | 3640 | 5 | 1.13e−007 |
| 3097 | 33 | 238 | 346, 416 | hsa-miR-24 | 1188 | 5 | 3.03e−007 |
| 3098 | 33 | 256 | 441, 487 | hsa-miR-26a | 2286 | 5 | 2.47e−008 |
| 3099 | 33 | 259 | 481 | hsa-miR-27a | 589 | 5 | 1.7e−008 |
| 3100 | 33 | 167 | 556 | hsa-miR-29a | 1094 | 5 | 3.47e−008 |
| 3101 | 33 | 241 | 550 | hsa-miR-30d | 926 | 5 | 1.74e−009 |
| 3102 | 33 | 141 | 437 | hsa-miR-33 | 547 | 5 | 5.33e−009 |
| 3103 | 33 | 208 | 477 | hsa-miR-342 | 562 | 5 | 1.3e−009 |
| 3104 | 33 | 63 | 557 | hsa-miR-491 | 532 | 5 | 7.85e−009 |
| 3105 | 33 | 214 | 376 | hsa-miR-494 | 3630 | 5 | 5.03e−006 |
| 3106 | 33 | 135 | 315 | hsa-miR-565 | 546 | 5 | 1.13e−008 |
| 3107 | 33 | 60 | 479 | hsa-miR-638 | 1571 | 5 | 5.13e−009 |
| 3108 | 33 | 182 | 304, 317 | hsa-miR-92 | 3270 | 5 | 1.37e−007 |
| 3109 | 33 | 14 | 318 | hsa-miR-99a | 1110 | 5 | 4.82e−007 |
| 3110 | 34 | 127 | 361 | 11_1 | 943 | 8 | 1.41e−022 |
| 3111 | 34 | 196 | 508 | 15_rel | 54465 | 8 | 1.04e−024 |
| 3112 | 34 | 197 | 444 | 25_6 | 683 | 8 | 2.74e−022 |
| 3113 | 34 | 219 | 497 | 27_3 | 2886 | 8 | 1.44e−022 |
| 3114 | 34 | 122 | 281 | 43_11 | 657 | 8 | 7.36e−023 |
| 3115 | 34 | 7 | 337 | 43_21 | 681 | 8 | 1.85e−022 |
| 3116 | 34 | 42 | 449 | 43_3 | 867 | 8 | 2.02e−023 |
| 3117 | 34 | 101 | 475 | 43_30 | 760 | 8 | 1.23e−022 |
| 3118 | 34 | 99 | 348, 384, 434 | 43_35 | 747 | 8 | 2.01e−022 |
| 3119 | 34 | 129 | 548 | 43_39 | 2794 | 8 | 6.2e−024 |
| 3120 | 34 | 27 | 313 | 43_40 | 616 | 8 | 2.45e−019 |
| 3121 | 34 | 211 | 353 | 43_44 | 579 | 8 | 7.88e−023 |
| 3122 | 34 | 145 | 447 | 43_44_rel | 53867 | 3 | 4.31e−009 |
| 3123 | 34 | 114 | 530 | 43_6 | 984 | 8 | 1.77e−022 |
| 3124 | 34 | 183 | 373 | 66_1 | 1241 | 8 | 1.81e−023 |
| 3125 | 34 | 94 | 394 | 69_6_rel | 22615 | 8 | 3.37e−022 |
| 3126 | 34 | 121 | 502 | 70_14 | 855 | 8 | 4.75e−021 |
| 3127 | 34 | 177 | 366, 464 | 72_12 | 2521 | 8 | 2.69e−023 |
| 3128 | 34 | 40 | 553 | 72_4 | 2362 | 8 | 1e−020 |
| 3129 | 34 | 97 | 357 | 74_26_rel | 4581 | 8 | 1.5e−020 |
| 3130 | 34 | 33 | 339, 552 | 96_rel | 48668 | 3 | 2.43e−008 |
| 3131 | 34 | 87 | 301 | gam11 | 8120 | 8 | 3.58e−017 |
| 3132 | 34 | 125 | 532 | 70_16 | 624 | 8 | 2.25e−019 |
| 3133 | 34 | 163 | 402 | 43_7 | 1339 | 8 | 1.28e−021 |
| 3134 | 34 | 223 | 320, 393, 559 | hsa-let-7a | 10410 | 8 | 1.31e−022 |
| 3135 | 34 | 225 | 332 | hsa-let-7b | 21582 | 8 | 1.32e−019 |
| 3136 | 34 | 224 | 427 | hsa-let-7c | 21049 | 8 | 7.51e−024 |
| 3137 | 34 | 45 | 277 | hsa-let-7d | 3264 | 8 | 3.22e−022 |
| 3138 | 34 | 220 | 475 | hsa-let-7e | 4706 | 8 | 1.51e−022 |
| 3139 | 34 | 222 | 482, 558 | hsa-let-7f | 865 | 8 | 2.9e−022 |
| 3140 | 34 | 226 | 395 | hsa-let-7g | 2183 | 8 | 8.82e−023 |
| 3141 | 34 | 227 | 297 | hsa-let-7i | 2647 | 8 | 2.67e−023 |
| 3142 | 34 | 231 | 321, 424 | hsa-miR-1 | 986 | 8 | 5.88e−022 |
| 3143 | 34 | 13 | 397 | hsa-miR-100 | 3514 | 8 | 1.57e−021 |
| 3144 | 34 | 48 | 483, 529 | hsa-miR-103 | 2231 | 8 | 5.31e−022 |
| 3145 | 34 | 184 | 423 | hsa-miR-105 | 1622 | 8 | 6.95e−024 |
| 3146 | 34 | 2 | 399 | hsa-miR-106a | 2103 | 8 | 5.78e−021 |
| 3147 | 34 | 149 | 392 | hsa-miR-106b | 1693 | 8 | 1.72e−023 |
| 3148 | 34 | 47 | 555 | hsa-miR-107 | 2175 | 8 | 6.56e−023 |
| 3149 | 34 | 199 | 387 | hsa-miR-125a | 3922 | 8 | 1.08e−022 |
| 3150 | 34 | 198 | 313, 551 | hsa-miR-125b | 16900 | 8 | 1.32e−020 |
| 3151 | 34 | 204 | 419 | hsa-miR-126 | 1781 | 8 | 1.99e−022 |
| 3152 | 34 | 89 | 419 | hsa-miR-126* | 967 | 8 | 1.71e−022 |
| 3153 | 34 | 86 | 417 | hsa-miR-130a | 3053 | 8 | 2.37e−023 |
| 3154 | 34 | 85 | 328 | hsa-miR-130b | 683 | 8 | 3.39e−022 |
| 3155 | 34 | 153 | 445 | hsa-miR-132 | 582 | 8 | 6.64e−025 |
| 3156 | 34 | 266 | 295, 386 | hsa-miR-133a | 1624 | 8 | 1.32e−021 |
| 3157 | 34 | 265 | 533 | hsa-miR-133b | 1457 | 8 | 1.07e−022 |
| 3158 | 34 | 181 | 324, 554 | hsa-miR-135a | 787 | 8 | 6.56e−022 |
| 3159 | 34 | 180 | 486 | hsa-miR-135b | 645 | 8 | 1.14e−021 |
| 3160 | 34 | 37 | 391 | hsa-miR-136 | 593 | 8 | 2.19e−019 |
| 3161 | 34 | 52 | 370, 548 | hsa-miR-138 | 1168 | 8 | 3.21e−022 |
| 3162 | 34 | 64 | 402 | hsa-miR-140 | 1221 | 8 | 1.88e−022 |
| 3163 | 34 | 151 | 472 | hsa-miR-141 | 3943 | 8 | 1.59e−021 |
| 3164 | 34 | 247 | 278 | hsa-miR-142-3p | 568 | 8 | 1.09e−021 |
| 3165 | 34 | 218 | 527 | hsa-miR-143 | 36584 | 8 | 3.27e−022 |
| 3166 | 34 | 139 | 281 | hsa-miR-145 | 45762 | 8 | 1.29e−024 |
| 3167 | 34 | 217 | 466 | hsa-miR-146a | 517 | 8 | 1.64e−023 |
| 3168 | 34 | 146 | 325 | hsa-miR-147 | 825 | 8 | 1.02e−022 |
| 3169 | 34 | 190 | 458 | hsa-miR-148a | 617 | 8 | 1.65e−022 |
| 3170 | 34 | 191 | 289 | hsa-miR-148b | 612 | 8 | 1.41e−023 |
| 3171 | 34 | 212 | 333 | hsa-miR-149 | 1424 | 8 | 5.89e−023 |
| 3172 | 34 | 209 | 507 | hsa-miR-150 | 727 | 8 | 3.38e−023 |
| 3173 | 34 | 34 | 396 | hsa-miR-151 | 977 | 8 | 9.91e−023 |
| 3174 | 34 | 192 | 470 | hsa-miR-152 | 831 | 8 | 1.51e−023 |
| 3175 | 34 | 263 | 296, 404 | hsa-miR-153 | 697 | 8 | 4.22e−022 |
| 3176 | 34 | 253 | 499 | hsa-miR-155 | 630 | 8 | 2.94e−022 |
| 3177 | 34 | 171 | 331 | hsa-miR-15a | 682 | 8 | 2.96e−024 |
| 3178 | 34 | 172 | 329 | hsa-miR-15b | 912 | 8 | 3.04e−025 |
| 3179 | 34 | 173 | 360, 468 | hsa-miR-16 | 3574 | 8 | 8.21e−023 |
| 3180 | 34 | 38 | 467 | hsa-miR-17-3p | 712 | 8 | 7.28e−025 |
| 3181 | 34 | 75 | 467 | hsa-miR-17-5p | 1770 | 8 | 8.61e−024 |
| 3182 | 34 | 10 | 371, 461 | hsa-miR-181a | 1275 | 8 | 9.82e−024 |
| 3183 | 34 | 179 | 356 | hsa-miR-183 | 1627 | 8 | 1.32e−022 |
| 3184 | 34 | 233 | 305 | hsa-miR-184 | 599 | 8 | 3.07e−022 |
| 3185 | 34 | 234 | 450 | hsa-miR-185 | 832 | 8 | 6.19e−025 |
| 3186 | 34 | 205 | 367 | hsa-miR-187 | 511 | 8 | 5.94e−023 |
| 3187 | 34 | 88 | 310 | hsa-miR-188 | 867 | 8 | 4.81e−022 |
| 3188 | 34 | 144 | 346 | hsa-miR-189 | 865 | 8 | 2.42e−024 |
| 3189 | 34 | 228 | 435 | hsa-miR-190 | 538 | 8 | 1.61e−023 |
| 3190 | 34 | 77 | 440 | hsa-miR-191 | 2028 | 8 | 2.4e−021 |
| 3191 | 34 | 130 | 440 | hsa-miR-191* | 1030 | 8 | 2.09e−021 |
| 3192 | 34 | 109 | 374 | hsa-miR-192 | 580 | 8 | 4.07e−023 |
| 3193 | 34 | 17 | 442 | hsa-miR-193a | 2979 | 8 | 1.36e−020 |
| 3194 | 34 | 16 | 488 | hsa-miR-193b | 894 | 8 | 6.86e−019 |
| 3195 | 34 | 170 | 327 | hsa-miR-195 | 1180 | 8 | 8.81e−021 |
| 3196 | 34 | 136 | 308 | hsa-miR-198 | 851 | 8 | 2.29e−023 |
| 3197 | 34 | 92 | 410, 432 | hsa-miR-199a | 4394 | 8 | 3.28e−022 |
| 3198 | 34 | 162 | 410, 432, 473 | hsa-miR-199a* | 670 | 8 | 6.93e−019 |
| 3199 | 34 | 93 | 473 | hsa-miR-199b | 649 | 8 | 2.43e−022 |
| 3200 | 34 | 251 | 282, 495 | hsa-miR-19b | 1483 | 8 | 1.29e−022 |
| 3201 | 34 | 152 | 369 | hsa-miR-200a | 904 | 8 | 6.13e−019 |
| 3202 | 34 | 157 | 411 | hsa-miR-200b | 2051 | 8 | 9.35e−022 |
| 3203 | 34 | 156 | 446 | hsa-miR-200c | 6001 | 8 | 9.97e−022 |
| 3204 | 34 | 271 | 334 | hsa-miR-202* | 1063 | 8 | 7.78e−023 |
| 3205 | 34 | 203 | 504 | hsa-miR-205 | 8238 | 8 | 2.09e−020 |
| 3206 | 34 | 232 | 422 | hsa-miR-206 | 1547 | 8 | 6.58e−021 |
| 3207 | 34 | 65 | 510 | hsa-miR-208 | 525 | 8 | 7.36e−023 |
| 3208 | 34 | 175 | 462 | hsa-miR-21 | 4666 | 8 | 4.12e−022 |
| 3209 | 34 | 115 | 314 | hsa-miR-210 | 794 | 8 | 1.2e−022 |
| 3210 | 34 | 154 | 300 | hsa-miR-212 | 843 | 8 | 1e−021 |
| 3211 | 34 | 29 | 439 | hsa-miR-214 | 2835 | 8 | 3.01e−022 |
| 3212 | 34 | 159 | 494 | hsa-miR-216 | 1192 | 8 | 1.59e−022 |
| 3213 | 34 | 268 | 401, 425 | hsa-miR-218 | 812 | 8 | 6.01e−023 |
| 3214 | 34 | 18 | 322 | hsa-miR-222 | 981 | 8 | 1.47e−021 |
| 3215 | 34 | 50 | 364 | hsa-miR-221 | 5398 | 8 | 1.23e−020 |
| 3216 | 34 | 49 | 302 | hsa-miR-222 | 8095 | 8 | 5.32e−022 |
| 3217 | 34 | 248 | 415 | hsa-miR-223 | 667 | 8 | 2.42e−024 |
| 3218 | 34 | 67 | 336 | hsa-miR-23a | 3281 | 8 | 2.23e−021 |
| 3219 | 34 | 66 | 306 | hsa-miR-23b | 1490 | 8 | 3.23e−016 |
| 3220 | 34 | 238 | 346, 416 | hsa-miR-24 | 12753 | 8 | 3.11e−020 |
| 3221 | 34 | 90 | 388 | hsa-miR-25 | 804 | 8 | 5.23e−019 |
| 3222 | 34 | 256 | 441, 487 | hsa-miR-26a | 10647 | 8 | 2.56e−022 |
| 3223 | 34 | 257 | 409 | hsa-miR-26b | 842 | 8 | 6.4e−019 |
| 3224 | 34 | 259 | 481 | hsa-miR-27a | 4134 | 8 | 1.42e−021 |
| 3225 | 34 | 260 | 280 | hsa-miR-27b | 3999 | 8 | 6.48e−022 |
| 3226 | 34 | 61 | 286 | hsa-miR-296 | 2096 | 8 | 4.13e−018 |
| 3227 | 34 | 167 | 556 | hsa-miR-29a | 693 | 8 | 5.23e−017 |
| 3228 | 34 | 269 | 362 | hsa-miR-302c* | 963 | 8 | 5.94e−023 |
| 3229 | 34 | 117 | 287 | hsa-miR-30a-3p | 1089 | 8 | 7.99e−023 |
| 3230 | 34 | 242 | 311 | hsa-miR-30b | 930 | 8 | 3.54e−022 |
| 3231 | 34 | 243 | 299, 316 | hsa-miR-30c | 1930 | 8 | 2.78e−021 |
| 3232 | 34 | 241 | 550 | hsa-miR-30d | 2362 | 8 | 1.14e−022 |
| 3233 | 34 | 116 | 431 | hsa-miR-30e-3p | 827 | 8 | 5.46e−023 |
| 3234 | 34 | 245 | 431 | hsa-miR-30e-5p | 2553 | 8 | 8.5e−024 |
| 3235 | 34 | 132 | 429 | hsa-miR-31 | 1522 | 8 | 1.83e−022 |
| 3236 | 34 | 1 | 359 | hsa-miR-320 | 5093 | 8 | 1.88e−021 |
| 3237 | 34 | 124 | 455 | hsa-miR-323 | 623 | 8 | 3.88e−019 |
| 3238 | 34 | 91 | 368 | hsa-miR-324-3p | 1350 | 8 | 1.16e−024 |
| 3239 | 34 | 98 | 368 | hsa-miR-324-5p | 1428 | 8 | 1.11e−023 |
| 3240 | 34 | 95 | 279 | hsa-miR-325 | 525 | 8 | 7.47e−024 |
| 3241 | 34 | 113 | 538 | hsa-miR-328 | 1477 | 8 | 1.4e−023 |
| 3242 | 34 | 28 | 544, 545 | hsa-miR-329 | 553 | 8 | 6.33e−023 |
| 3243 | 34 | 141 | 437 | hsa-miR-33 | 2059 | 8 | 3.26e−023 |
| 3244 | 34 | 126 | 380 | hsa-miR-331 | 816 | 8 | 1e−023 |
| 3245 | 34 | 185 | 492 | hsa-miR-335 | 795 | 8 | 1.76e−023 |
| 3246 | 34 | 194 | 337 | hsa-miR-338 | 638 | 8 | 7.06e−020 |
| 3247 | 34 | 208 | 477 | hsa-miR-342 | 1584 | 8 | 7.63e−025 |
| 3248 | 34 | 229 | 292 | hsa-miR-345 | 1257 | 8 | 3.1e−024 |
| 3249 | 34 | 237 | 480 | hsa-miR-34a | 2400 | 8 | 5.26e−021 |
| 3250 | 34 | 255 | 444 | hsa-miR-361 | 752 | 8 | 3.6e−019 |
| 3251 | 34 | 26 | 493 | hsa-miR-363 | 581 | 8 | 1.09e−021 |
| 3252 | 34 | 160 | 379, 382 | hsa-miR-365 | 1150 | 8 | 8.41e−024 |
| 3253 | 34 | 128 | 294 | hsa-miR-370 | 1594 | 8 | 3.11e−022 |
| 3254 | 34 | 35 | 406 | hsa-miR-373* | 925 | 8 | 8.48e−019 |
| 3255 | 34 | 254 | 496 | hsa-miR-374 | 889 | 8 | 3.39e−023 |
| 3256 | 34 | 105 | 372 | hsa-miR-378 | 711 | 8 | 1.72e−022 |
| 3257 | 34 | 178 | 352 | hsa-miR-381 | 614 | 8 | 1.33e−022 |
| 3258 | 34 | 46 | 298 | hsa-miR-383 | 757 | 8 | 6.87e−023 |
| 3259 | 34 | 111 | 389 | hsa-miR-422a | 840 | 8 | 2.85e−023 |
| 3260 | 34 | 112 | 372 | hsa-miR-422b | 1824 | 8 | 1.96e−021 |
| 3261 | 34 | 51 | 497 | hsa-miR-423 | 3313 | 8 | 5.05e−022 |
| 3262 | 34 | 82 | 377 | hsa-miR-424 | 604 | 8 | 3.21e−022 |
| 3263 | 34 | 69 | 363 | hsa-miR-425-3p | 638 | 8 | 2.57e−022 |
| 3264 | 34 | 213 | 531 | hsa-miR-432 | 787 | 8 | 9.97e−022 |
| 3265 | 34 | 264 | 541 | hsa-miR-448 | 556 | 8 | 4.33e−023 |
| 3266 | 34 | 236 | 413 | hsa-miR-449 | 4271 | 8 | 6.19e−021 |
| 3267 | 34 | 274 | 375, 309 | hsa-miR-450 | 806 | 8 | 2.09e−022 |
| 3268 | 34 | 252 | 405 | hsa-miR-452 | 1392 | 8 | 1.46e−021 |
| 3269 | 34 | 138 | 547 | hsa-miR-485-3p | 652 | 8 | 1.21e−022 |
| 3270 | 34 | 44 | 547 | hsa-miR-485-5p | 843 | 8 | 7.21e−019 |
| 3271 | 34 | 62 | 485 | hsa-miR-489 | 696 | 8 | 1.24e−023 |
| 3272 | 34 | 63 | 557 | hsa-miR-491 | 5293 | 8 | 3.5e−022 |
| 3273 | 34 | 54 | 293 | hsa-miR-492 | 1312 | 8 | 1.85e−022 |
| 3274 | 34 | 267 | 354 | hsa-miR-493-5p | 1046 | 8 | 1.46e−022 |
| 3275 | 34 | 214 | 376 | hsa-miR-494 | 9973 | 8 | 9.63e−020 |
| 3276 | 34 | 3 | 543 | hsa-miR-495 | 937 | 8 | 1.92e−021 |
| 3277 | 34 | 83 | 312 | hsa-miR-497 | 2886 | 8 | 8.98e−024 |
| 3278 | 34 | 270 | 390 | hsa-miR-498 | 552 | 8 | 1.98e−019 |
| 3279 | 34 | 25 | 414 | hsa-miR-501 | 804 | 8 | 1.78e−022 |
| 3280 | 34 | 68 | 288 | hsa-miR-502 | 717 | 8 | 3.65e−022 |
| 3281 | 34 | 43 | 418 | hsa-miR-504 | 1501 | 8 | 7.8e−022 |
| 3282 | 34 | 81 | 454 | hsa-miR-512-5p | 550 | 8 | 6.24e−023 |
| 3283 | 34 | 258 | 428, 443 | hsa-miR-513 | 2151 | 8 | 4.64e−022 |
| 3284 | 34 | 120 | 522 | hsa-miR-515-3p | 536 | 8 | 4.63e−024 |
| 3285 | 34 | 261 | 522 | hsa-miR-515-5p | 524 | 8 | 8.29e−024 |
| 3286 | 34 | 70 | 518 | hsa-miR-516-5p | 515 | 8 | 5.17e−023 |
| 3287 | 34 | 73 | 503 | hsa-miR-518b | 1018 | 8 | 5.62e−022 |
| 3288 | 34 | 210 | 400 | hsa-miR-518c* | 1364 | 8 | 2.2e−022 |
| 3289 | 34 | 262 | 523 | hsa-miR-519e* | 1126 | 8 | 1.88e−023 |
| 3290 | 34 | 102 | 344 | hsa-miR-520a* | 681 | 8 | 9.15e−025 |
| 3291 | 34 | 206 | 513 | hsa-miR-520d* | 1180 | 8 | 1.49e−023 |
| 3292 | 34 | 100 | 524 | hsa-miR-524* | 1036 | 8 | 5.53e−023 |
| 3293 | 34 | 103 | 342 | hsa-miR-525 | 585 | 8 | 1.83e−019 |
| 3294 | 34 | 118 | 342 | hsa-miR-525* | 618 | 8 | 4.2e−020 |
| 3295 | 34 | 108 | 501 | hsa-miR-526b | 673 | 8 | 2.82e−019 |
| 3296 | 34 | 107 | 343, 506, 515, 520, 521, 526 | hsa-miR-526c | 622 | 8 | 2.98e−019 |
| 3297 | 34 | 110 | 511, 512, 514 | hsa-miR-527 | 833 | 8 | 1e−022 |
| 3298 | 34 | 148 | 283 | hsa-miR-557 | 954 | 8 | 5.15e−023 |
| 3299 | 34 | 56 | 338 | hsa-miR-564 | 512 | 8 | 1.35e−020 |
| 3300 | 34 | 135 | 315 | hsa-miR-565 | 5066 | 8 | 4.95e−022 |
| 3301 | 34 | 39 | 539 | hsa-miR-637 | 533 | 8 | 7.95e−022 |
| 3302 | 34 | 60 | 479 | hsa-miR-638 | 26623 | 8 | 1.59e−022 |
| 3303 | 34 | 195 | 421 | hsa-miR-662 | 722 | 8 | 2.71e−021 |
| 3304 | 34 | 230 | 474, 484, 536 | hsa-miR-7 | 736 | 8 | 2.52e−022 |
| 3305 | 34 | 182 | 304, 317 | hsa-miR-92 | 2660 | 8 | 1.08e−022 |
| 3306 | 34 | 6 | 471 | hsa-miR-93 | 1532 | 8 | 5.1e−022 |
| 3307 | 34 | 14 | 318 | hsa-miR-99a | 12655 | 8 | 2.21e−020 |
| 3308 | 34 | 80 | 509 | hsa-miR-99b | 1634 | 8 | 3.83e−024 |
| 3309 | 34 | 22 | 453 | c14 + 290a | 512 | 8 | 1.77e−024 |
| 3310 | 34 | 131 | 430 | 6_11 | 1077 | 8 | 1.44e−022 |
| 3311 | 34 | 23 | 546 | 47_6 | 504 | 8 | 3.23e−022 |
| 3312 | 35 | 127 | 361 | 11_1 | 582 | 15 | 0 |
| 3313 | 35 | 196 | 508 | 15_rel | 54505 | 15 | 0 |
| 3314 | 35 | 219 | 497 | 27_3 | 2457 | 15 | 0 |
| 3315 | 35 | 42 | 449 | 43_3 | 529 | 15 | 0 |
| 3316 | 35 | 99 | 348, 384, 434 | 43_35 | 660 | 15 | 0 |
| 3317 | 35 | 129 | 548 | 43_39 | 1488 | 15 | 0 |
| 3318 | 35 | 114 | 530 | 43_6 | 605 | 15 | 0 |
| 3319 | 35 | 183 | 373 | 66_1 | 683 | 15 | 0 |
| 3320 | 35 | 94 | 394 | 69_6_rel | 6912 | 15 | 5.37e−031 |
| 3321 | 35 | 121 | 502 | 70_14 | 595 | 15 | 0 |
| 3322 | 35 | 177 | 366, 464 | 72_12 | 1305 | 15 | 4.52e−038 |
| 3323 | 35 | 40 | 553 | 72_4 | 1189 | 15 | 1.11e−035 |
| 3324 | 35 | 97 | 357 | 74_26_rel | 4529 | 15 | 0 |
| 3325 | 35 | 87 | 301 | gam11 | 1005 | 15 | 1.43e−029 |
| 3326 | 35 | 163 | 402 | 43_7 | 1085 | 15 | 0 |
| 3327 | 35 | 223 | 320, 393, 559 | hsa-let-7a | 4501 | 15 | 1.54e−037 |
| 3328 | 35 | 225 | 332 | hsa-let-7b | 26387 | 15 | 0 |
| 3329 | 35 | 224 | 427 | hsa-let-7c | 12873 | 15 | 0 |
| 3330 | 35 | 45 | 277 | hsa-let-7d | 2650 | 15 | 0 |
| 3331 | 35 | 220 | 475 | hsa-let-7e | 3059 | 15 | 0 |
| 3332 | 35 | 222 | 482, 558 | hsa-let-7f | 719 | 15 | 0 |
| 3333 | 35 | 226 | 395 | hsa-let-7g | 1629 | 15 | 0 |
| 3334 | 35 | 227 | 297 | hsa-let-7i | 1972 | 15 | 0 |
| 3335 | 35 | 231 | 321, 424 | hsa-miR-1 | 502 | 15 | 0 |
| 3336 | 35 | 13 | 397 | hsa-miR-100 | 1694 | 15 | 4.13e−033 |
| 3337 | 35 | 48 | 483, 529 | hsa-miR-103 | 669 | 15 | 2.2e−033 |
| 3338 | 35 | 184 | 423 | hsa-miR-105 | 836 | 15 | 0 |
| 3339 | 35 | 2 | 399 | hsa-miR-106a | 1267 | 15 | 0 |
| 3340 | 35 | 149 | 392 | hsa-miR-106b | 785 | 15 | 0 |
| 3341 | 35 | 47 | 555 | hsa-miR-107 | 1426 | 15 | 0 |
| 3342 | 35 | 199 | 387 | hsa-miR-125a | 2525 | 15 | 0 |
| 3343 | 35 | 198 | 313, 551 | hsa-miR-125b | 16231 | 15 | 0 |
| 3344 | 35 | 204 | 419 | hsa-miR-126 | 697 | 15 | 0 |
| 3345 | 35 | 86 | 417 | hsa-miR-130a | 1936 | 15 | 0 |
| 3346 | 35 | 266 | 295, 386 | hsa-miR-133a | 857 | 15 | 0 |
| 3347 | 35 | 265 | 533 | hsa-miR-133b | 1165 | 15 | 0 |
| 3348 | 35 | 64 | 402 | hsa-miR-140 | 576 | 15 | 1.11e−037 |
| 3349 | 35 | 151 | 472 | hsa-miR-141 | 2891 | 15 | 5.17e−038 |
| 3350 | 35 | 218 | 527 | hsa-miR-143 | 3689 | 15 | 3.06e−029 |
| 3351 | 35 | 139 | 281 | hsa-miR-145 | 46011 | 15 | 0 |
| 3352 | 35 | 146 | 325 | hsa-miR-147 | 605 | 15 | 0 |
| 3353 | 35 | 191 | 289 | hsa-miR-148b | 559 | 15 | 0 |
| 3354 | 35 | 212 | 333 | hsa-miR-149 | 1517 | 15 | 0 |
| 3355 | 35 | 209 | 507 | hsa-miR-150 | 573 | 15 | 0 |
| 3356 | 35 | 34 | 396 | hsa-miR-151 | 879 | 15 | 0 |
| 3357 | 35 | 192 | 470 | hsa-miR-152 | 761 | 15 | 0 |
| 3358 | 35 | 263 | 296, 404 | hsa-miR-153 | 637 | 15 | 0 |
| 3359 | 35 | 253 | 499 | hsa-miR-155 | 592 | 15 | 0 |
| 3360 | 35 | 173 | 360, 468 | hsa-miR-16 | 1289 | 15 | 4.26e−038 |
| 3361 | 35 | 75 | 467 | hsa-miR-17-5p | 782 | 15 | 0 |
| 3362 | 35 | 10 | 371, 461 | hsa-miR-181a | 860 | 15 | 0 |
| 3363 | 35 | 179 | 356 | hsa-miR-183 | 770 | 15 | 1.55e−034 |
| 3364 | 35 | 234 | 450 | hsa-miR-185 | 589 | 15 | 0 |
| 3365 | 35 | 88 | 310 | hsa-miR-188 | 581 | 15 | 0 |
| 3366 | 35 | 144 | 346 | hsa-miR-189 | 550 | 15 | 0 |
| 3367 | 35 | 77 | 440 | hsa-miR-191 | 1625 | 15 | 0 |
| 3368 | 35 | 130 | 440 | hsa-miR-191* | 734 | 15 | 0 |
| 3369 | 35 | 17 | 442 | hsa-miR-193a | 2161 | 15 | 0 |
| 3370 | 35 | 136 | 308 | hsa-miR-198 | 526 | 15 | 0 |
| 3371 | 35 | 92 | 410, 432 | hsa-miR-199a | 1928 | 15 | 1.68e−034 |
| 3372 | 35 | 157 | 411 | hsa-miR-200b | 633 | 15 | 4.4e−035 |
| 3373 | 35 | 156 | 446 | hsa-miR-200c | 5177 | 15 | 0 |
| 3374 | 35 | 271 | 334 | hsa-miR-202* | 644 | 15 | 0 |
| 3375 | 35 | 203 | 504 | hsa-miR-205 | 9846 | 15 | 3.81e−038 |
| 3376 | 35 | 232 | 422 | hsa-miR-206 | 1211 | 15 | 0 |
| 3377 | 35 | 175 | 462 | hsa-miR-21 | 3044 | 15 | 5.87e−037 |
| 3378 | 35 | 115 | 314 | hsa-miR-210 | 650 | 15 | 0 |
| 3379 | 35 | 154 | 300 | hsa-miR-212 | 653 | 15 | 0 |
| 3380 | 35 | 29 | 439 | hsa-miR-214 | 2365 | 15 | 0 |
| 3381 | 35 | 159 | 494 | hsa-miR-216 | 676 | 15 | 0 |
| 3382 | 35 | 268 | 401, 425 | hsa-miR-218 | 579 | 15 | 0 |
| 3383 | 35 | 18 | 322 | hsa-miR-22 | 2204 | 15 | 0 |
| 3384 | 35 | 50 | 364 | hsa-miR-221 | 4315 | 15 | 0 |
| 3385 | 35 | 49 | 302 | hsa-miR-222 | 7285 | 15 | 0 |
| 3386 | 35 | 67 | 336 | hsa-miR-23a | 1515 | 15 | 3.71e−038 |
| 3387 | 35 | 238 | 346, 416 | hsa-miR-24 | 9569 | 15 | 0 |
| 3388 | 35 | 256 | 441, 487 | hsa-miR-26a | 4979 | 15 | 0 |
| 3389 | 35 | 259 | 481 | hsa-miR-27a | 1546 | 15 | 2.4e−038 |
| 3390 | 35 | 260 | 280 | hsa-miR-27b | 1841 | 15 | 8.93e−038 |
| 3391 | 35 | 117 | 287 | hsa-miR-30a-3p | 573 | 15 | 0 |
| 3392 | 35 | 243 | 299, 316 | hsa-miR-30c | 1458 | 15 | 6.1e−038 |
| 3393 | 35 | 241 | 550 | hsa-miR-30d | 1746 | 15 | 0 |
| 3394 | 35 | 116 | 431 | hsa-miR-30e-3p | 576 | 15 | 0 |
| 3395 | 35 | 245 | 431 | hsa-miR-30e-5p | 2119 | 15 | 0 |
| 3396 | 35 | 132 | 429 | hsa-miR-31 | 1204 | 15 | 0 |
| 3397 | 35 | 1 | 359 | hsa-miR-320 | 4922 | 15 | 0 |
| 3398 | 35 | 124 | 455 | hsa-miR-323 | 713 | 15 | 0 |
| 3399 | 35 | 91 | 368 | hsa-miR-324-3p | 969 | 15 | 0 |
| 3400 | 35 | 98 | 368 | hsa-miR-324-5p | 1132 | 15 | 0 |
| 3401 | 35 | 113 | 538 | hsa-miR-328 | 931 | 15 | 0 |
| 3402 | 35 | 141 | 437 | hsa-miR-33 | 1352 | 15 | 0 |
| 3403 | 35 | 126 | 380 | hsa-miR-331 | 594 | 15 | 0 |
| 3404 | 35 | 208 | 477 | hsa-miR-342 | 1219 | 15 | 0 |
| 3405 | 35 | 229 | 292 | hsa-miR-345 | 850 | 15 | 0 |
| 3406 | 35 | 237 | 480 | hsa-miR-34a | 1166 | 15 | 0 |
| 3407 | 35 | 160 | 379, 382 | hsa-miR-365 | 754 | 15 | 0 |
| 3408 | 35 | 128 | 294 | hsa-miR-370 | 830 | 15 | 0 |
| 3409 | 35 | 254 | 496 | hsa-miR-374 | 610 | 15 | 0 |
| 3410 | 35 | 46 | 298 | hsa-miR-383 | 531 | 15 | 0 |
| 3411 | 35 | 111 | 389 | hsa-miR-422a | 637 | 15 | 0 |
| 3412 | 35 | 112 | 372 | hsa-miR-422b | 1683 | 15 | 0 |
| 3413 | 35 | 51 | 497 | hsa-miR-423 | 2700 | 15 | 0 |
| 3414 | 35 | 69 | 363 | hsa-miR-425-3p | 596 | 15 | 0 |
| 3415 | 35 | 213 | 531 | hsa-miR-432 | 666 | 15 | 0 |
| 3416 | 35 | 236 | 413 | hsa-miR-449 | 3756 | 15 | 0 |
| 3417 | 35 | 274 | 375, 309 | hsa-miR-450 | 604 | 15 | 0 |
| 3418 | 35 | 252 | 405 | hsa-miR-452 | 1162 | 15 | 0 |
| 3419 | 35 | 138 | 547 | hsa-miR-485-3p | 589 | 15 | 0 |
| 3420 | 35 | 63 | 557 | hsa-miR-491 | 3600 | 15 | 0 |
| 3421 | 35 | 54 | 293 | hsa-miR-492 | 915 | 15 | 0 |
| 3422 | 35 | 267 | 354 | hsa-miR-493-5p | 708 | 15 | 0 |
| 3423 | 35 | 214 | 376 | hsa-miR-494 | 13910 | 15 | 0 |
| 3424 | 35 | 3 | 543 | hsa-miR-495 | 829 | 15 | 0 |
| 3425 | 35 | 83 | 312 | hsa-miR-497 | 1509 | 15 | 0 |
| 3426 | 35 | 25 | 414 | hsa-miR-501 | 569 | 15 | 0 |
| 3427 | 35 | 43 | 418 | hsa-miR-504 | 979 | 15 | 0 |
| 3428 | 35 | 258 | 428, 443 | hsa-miR-513 | 1703 | 15 | 0 |
| 3429 | 35 | 73 | 503 | hsa-miR-518b | 769 | 15 | 0 |
| 3430 | 35 | 210 | 400 | hsa-miR-518c* | 842 | 15 | 0 |
| 3431 | 35 | 262 | 523 | hsa-miR-519e* | 693 | 15 | 0 |
| 3432 | 35 | 206 | 513 | hsa-miR-520d* | 613 | 15 | 3.84e−036 |
| 3433 | 35 | 100 | 524 | hsa-miR-524* | 687 | 15 | 0 |
| 3434 | 35 | 118 | 342 | hsa-miR-525* | 832 | 15 | 1.48e−038 |
| 3435 | 35 | 110 | 511, 512, 514 | hsa-miR-527 | 571 | 15 | 0 |
| 3436 | 35 | 135 | 315 | hsa-miR-565 | 3647 | 15 | 1.33e−038 |
| 3437 | 35 | 60 | 479 | hsa-miR-638 | 19003 | 15 | 0 |
| 3438 | 35 | 230 | 474, 484, 536 | hsa-miR-7 | 759 | 15 | 0 |
| 3439 | 35 | 182 | 304, 317 | hsa-miR-92 | 1736 | 15 | 0 |
| 3440 | 35 | 6 | 471 | hsa-miR-93 | 1018 | 15 | 0 |
| 3441 | 35 | 14 | 318 | hsa-miR-99a | 12231 | 15 | 0 |
| 3442 | 35 | 80 | 509 | hsa-miR-99b | 1307 | 15 | 0 |
| 3443 | 35 | 131 | 430 | 6_11 | 596 | 15 | 0 |
| 3444 | 36 | 196 | 508 | 15_rel | 41153 | 33 | 0 |
| 3445 | 36 | 197 | 444 | 25_6 | 582 | 33 | 0 |
| 3446 | 36 | 219 | 497 | 27_3 | 1690 | 33 | 0 |
| 3447 | 36 | 42 | 449 | 43_3 | 525 | 33 | 0 |
| 3448 | 36 | 101 | 475 | 43_30 | 565 | 33 | 0 |
| 3449 | 36 | 129 | 548 | 43_39 | 1846 | 33 | 0 |
| 3450 | 36 | 114 | 530 | 43_6 | 531 | 33 | 0 |
| 3451 | 36 | 183 | 373 | 66_1 | 736 | 33 | 0 |
| 3452 | 36 | 94 | 394 | 69_6_rel | 6873 | 33 | 0 |
| 3453 | 36 | 177 | 366, 464 | 72_12 | 1217 | 33 | 0 |
| 3454 | 36 | 40 | 553 | 72_4 | 1127 | 33 | 0 |
| 3455 | 36 | 97 | 357 | 74_26_rel | 2705 | 33 | 0 |
| 3456 | 36 | 163 | 402 | 43_7 | 1095 | 33 | 0 |
| 3457 | 36 | 223 | 320, 393, 559 | hsa-let-7a | 4303 | 33 | 0 |
| 3458 | 36 | 225 | 332 | hsa-let-7b | 14557 | 33 | 0 |
| 3459 | 36 | 224 | 427 | hsa-let-7c | 10976 | 33 | 0 |
| 3460 | 36 | 45 | 277 | hsa-let-7d | 2071 | 33 | 0 |
| 3461 | 36 | 220 | 475 | hsa-let-7e | 2804 | 33 | 0 |
| 3462 | 36 | 222 | 482, 558 | hsa-let-7f | 541 | 33 | 0 |
| 3463 | 36 | 226 | 395 | hsa-let-7g | 1522 | 33 | 0 |
| 3464 | 36 | 227 | 297 | hsa-let-7i | 1427 | 33 | 0 |
| 3465 | 36 | 231 | 321, 424 | hsa-miR-1 | 516 | 33 | 0 |
| 3466 | 36 | 13 | 397 | hsa-miR-100 | 1684 | 33 | 0 |
| 3467 | 36 | 48 | 483, 529 | hsa-miR-103 | 924 | 33 | 0 |
| 3468 | 36 | 184 | 423 | hsa-miR-105 | 851 | 33 | 0 |
| 3469 | 36 | 2 | 399 | hsa-miR-106a | 1231 | 33 | 0 |
| 3470 | 36 | 149 | 392 | hsa-miR-106b | 1015 | 33 | 0 |
| 3471 | 36 | 47 | 555 | hsa-miR-107 | 1528 | 33 | 0 |
| 3472 | 36 | 199 | 387 | hsa-miR-125a | 2394 | 33 | 0 |
| 3473 | 36 | 198 | 313, 551 | hsa-miR-125b | 13499 | 33 | 0 |
| 3474 | 36 | 204 | 419 | hsa-miR-126 | 1385 | 33 | 0 |
| 3475 | 36 | 89 | 419 | hsa-miR-126* | 610 | 33 | 0 |
| 3476 | 36 | 86 | 417 | hsa-miR-130a | 2277 | 33 | 0 |
| 3477 | 36 | 266 | 295, 386 | hsa-miR-133a | 1755 | 33 | 0 |
| 3478 | 36 | 265 | 533 | hsa-miR-133b | 1715 | 33 | 0 |
| 3479 | 36 | 180 | 486 | hsa-miR-135b | 536 | 33 | 0 |
| 3480 | 36 | 52 | 370, 548 | hsa-miR-138 | 653 | 33 | 0 |
| 3481 | 36 | 64 | 402 | hsa-miR-140 | 827 | 33 | 0 |
| 3482 | 36 | 151 | 472 | hsa-miR-141 | 1409 | 33 | 0 |
| 3483 | 36 | 218 | 527 | hsa-miR-143 | 2424 | 33 | 0 |
| 3484 | 36 | 139 | 281 | hsa-miR-145 | 34454 | 33 | 0 |
| 3485 | 36 | 146 | 325 | hsa-miR-147 | 548 | 33 | 0 |
| 3486 | 36 | 212 | 333 | hsa-miR-149 | 900 | 33 | 0 |
| 3487 | 36 | 209 | 507 | hsa-miR-150 | 689 | 33 | 0 |
| 3488 | 36 | 34 | 396 | hsa-miR-151 | 617 | 33 | 0 |
| 3489 | 36 | 192 | 470 | hsa-miR-152 | 678 | 33 | 0 |
| 3490 | 36 | 173 | 360, 468 | hsa-miR-16 | 2495 | 33 | 0 |
| 3491 | 36 | 75 | 467 | hsa-miR-17-5p | 928 | 33 | 0 |
| 3492 | 36 | 10 | 371, 461 | hsa-miR-181a | 862 | 33 | 0 |
| 3493 | 36 | 234 | 450 | hsa-miR-185 | 648 | 33 | 0 |
| 3494 | 36 | 88 | 310 | hsa-miR-188 | 591 | 33 | 0 |
| 3495 | 36 | 144 | 346 | hsa-miR-189 | 611 | 33 | 0 |
| 3496 | 36 | 77 | 440 | hsa-miR-191 | 1510 | 33 | 0 |
| 3497 | 36 | 130 | 440 | hsa-miR-191* | 761 | 33 | 0 |
| 3498 | 36 | 17 | 442 | hsa-miR-193a | 2181 | 33 | 0 |
| 3499 | 36 | 170 | 327 | hsa-miR-195 | 784 | 33 | 0 |
| 3500 | 36 | 136 | 308 | hsa-miR-198 | 527 | 33 | 0 |
| 3501 | 36 | 92 | 410, 432 | hsa-miR-199a | 3258 | 33 | 0 |
| 3502 | 36 | 162 | 410, 432, 473 | hsa-miR-199a* | 830 | 33 | 0 |
| 3503 | 36 | 93 | 473 | hsa-miR-199b | 501 | 33 | 0 |
| 3504 | 36 | 157 | 411 | hsa-miR-200b | 769 | 33 | 0 |
| 3505 | 36 | 156 | 446 | hsa-miR-200c | 1927 | 33 | 0 |
| 3506 | 36 | 271 | 334 | hsa-miR-202* | 732 | 33 | 0 |
| 3507 | 36 | 203 | 504 | hsa-miR-205 | 3935 | 33 | 0 |
| 3508 | 36 | 232 | 422 | hsa-miR-206 | 789 | 33 | 0 |
| 3509 | 36 | 175 | 462 | hsa-miR-21 | 2311 | 33 | 0 |
| 3510 | 36 | 115 | 314 | hsa-miR-210 | 553 | 33 | 0 |
| 3511 | 36 | 154 | 300 | hsa-miR-212 | 501 | 33 | 0 |
| 3512 | 36 | 29 | 439 | hsa-miR-214 | 2205 | 33 | 0 |
| 3513 | 36 | 159 | 494 | hsa-miR-216 | 687 | 33 | 0 |
| 3514 | 36 | 268 | 401, 425 | hsa-miR-218 | 594 | 33 | 0 |
| 3515 | 36 | 18 | 322 | hsa-miR-22 | 2608 | 33 | 0 |
| 3516 | 36 | 50 | 364 | hsa-miR-221 | 4293 | 33 | 0 |
| 3517 | 36 | 49 | 302 | hsa-miR-222 | 5767 | 33 | 0 |
| 3518 | 36 | 248 | 415 | hsa-miR-223 | 520 | 33 | 0 |
| 3519 | 36 | 67 | 336 | hsa-miR-23a | 2995 | 33 | 0 |
| 3520 | 36 | 238 | 346, 416 | hsa-miR-24 | 11639 | 33 | 0 |
| 3521 | 36 | 256 | 441, 487 | hsa-miR-26a | 8472 | 33 | 0 |
| 3522 | 36 | 257 | 409 | hsa-miR-26b | 548 | 33 | 0 |
| 3523 | 36 | 259 | 481 | hsa-miR-27a | 3841 | 33 | 0 |
| 3524 | 36 | 260 | 280 | hsa-miR-27b | 2962 | 33 | 0 |
| 3525 | 36 | 117 | 287 | hsa-miR-30a-3p | 680 | 33 | 0 |
| 3526 | 36 | 242 | 311 | hsa-miR-30b | 633 | 33 | 0 |
| 3527 | 36 | 243 | 299, 316 | hsa-miR-30c | 1372 | 33 | 0 |
| 3528 | 36 | 241 | 550 | hsa-miR-30d | 1530 | 33 | 0 |
| 3529 | 36 | 116 | 431 | hsa-miR-30e-3p | 560 | 33 | 0 |
| 3530 | 36 | 245 | 431 | hsa-miR-30e-5p | 1482 | 33 | 0 |
| 3531 | 36 | 132 | 429 | hsa-miR-31 | 860 | 33 | 0 |
| 3532 | 36 | 1 | 359 | hsa-miR-320 | 3726 | 33 | 0 |
| 3533 | 36 | 91 | 368 | hsa-miR-324-3p | 1048 | 33 | 0 |
| 3534 | 36 | 98 | 368 | hsa-miR-324-5p | 1070 | 33 | 0 |
| 3535 | 36 | 113 | 538 | hsa-miR-328 | 1349 | 33 | 0 |
| 3536 | 36 | 141 | 437 | hsa-miR-33 | 1146 | 33 | 0 |
| 3537 | 36 | 126 | 380 | hsa-miR-331 | 623 | 33 | 0 |
| 3538 | 36 | 208 | 477 | hsa-miR-342 | 1110 | 33 | 0 |
| 3539 | 36 | 229 | 292 | hsa-miR-345 | 840 | 33 | 0 |
| 3540 | 36 | 237 | 480 | hsa-miR-34a | 1597 | 33 | 0 |
| 3541 | 36 | 160 | 379, 382 | hsa-miR-365 | 1202 | 33 | 0 |
| 3542 | 36 | 128 | 294 | hsa-miR-370 | 925 | 33 | 0 |
| 3543 | 36 | 254 | 496 | hsa-miR-374 | 516 | 33 | 0 |
| 3544 | 36 | 111 | 389 | hsa-miR-422a | 602 | 33 | 0 |
| 3545 | 36 | 112 | 372 | hsa-miR-422b | 1399 | 33 | 0 |
| 3546 | 36 | 51 | 497 | hsa-miR-423 | 2667 | 33 | 0 |
| 3547 | 36 | 69 | 363 | hsa-miR-425-3p | 549 | 33 | 0 |
| 3548 | 36 | 236 | 413 | hsa-miR-449 | 2241 | 33 | 0 |
| 3549 | 36 | 252 | 405 | hsa-miR-452 | 782 | 33 | 0 |
| 3550 | 36 | 63 | 557 | hsa-miR-491 | 3156 | 33 | 0 |
| 3551 | 36 | 54 | 293 | hsa-miR-492 | 852 | 33 | 0 |
| 3552 | 36 | 214 | 376 | hsa-miR-494 | 7963 | 33 | 0 |
| 3553 | 36 | 3 | 543 | hsa-miR-495 | 531 | 33 | 0 |
| 3554 | 36 | 83 | 312 | hsa-miR-497 | 1670 | 33 | 0 |
| 3555 | 36 | 43 | 418 | hsa-miR-504 | 1084 | 33 | 0 |
| 3556 | 36 | 258 | 428, 443 | hsa-miR-513 | 1504 | 33 | 0 |
| 3557 | 36 | 73 | 503 | hsa-miR-518b | 704 | 33 | 0 |
| 3558 | 36 | 210 | 400 | hsa-miR-518c* | 938 | 33 | 0 |
| 3559 | 36 | 262 | 523 | hsa-miR-519e* | 656 | 33 | 0 |
| 3560 | 36 | 206 | 513 | hsa-miR-520d* | 809 | 33 | 0 |
| 3561 | 36 | 100 | 524 | hsa-miR-524* | 670 | 33 | 0 |
| 3562 | 36 | 118 | 342 | hsa-miR-525* | 569 | 33 | 0 |
| 3563 | 36 | 110 | 511, 512, 514 | hsa-miR-527 | 546 | 33 | 0 |
| 3564 | 36 | 148 | 283 | hsa-miR-557 | 530 | 33 | 0 |
| 3565 | 36 | 135 | 315 | hsa-miR-565 | 2006 | 33 | 0 |
| 3566 | 36 | 12 | 275 | hsa-miR-634 | 612 | 33 | 0 |
| 3567 | 36 | 60 | 479 | hsa-miR-638 | 14716 | 33 | 0 |
| 3568 | 36 | 182 | 304, 317 | hsa-miR-92 | 1405 | 33 | 0 |
| 3569 | 36 | 6 | 471 | hsa-miR-93 | 971 | 33 | 0 |
| 3570 | 36 | 14 | 318 | hsa-miR-99a | 9534 | 33 | 0 |
| 3571 | 36 | 80 | 509 | hsa-miR-99b | 1253 | 33 | 0 |
| 3572 | 36 | 131 | 430 | 6_11 | 697 | 33 | 0 |
| 3573 | 37 | 127 | 361 | 11_1 | 1206 | 30 | 0 |
| 3574 | 37 | 196 | 508 | 15_rel | 38401 | 30 | 0 |
| 3575 | 37 | 134 | 436 | 24_5_rel | 56448 | 4 | 8.51e−012 |
| 3576 | 37 | 219 | 497 | 27_3 | 2392 | 30 | 0 |
| 3577 | 37 | 122 | 281 | 43_11 | 522 | 30 | 0 |
| 3578 | 37 | 42 | 449 | 43_3 | 544 | 30 | 0 |
| 3579 | 37 | 99 | 348, 384, 434 | 43_35 | 588 | 30 | 0 |
| 3580 | 37 | 79 | 460 | 43_37_rel | 37054 | 4 | 9.94e−010 |
| 3581 | 37 | 129 | 548 | 43_39 | 1692 | 30 | 0 |
| 3582 | 37 | 145 | 447 | 43_44_rel | 56086 | 5 | 2.08e−015 |
| 3583 | 37 | 114 | 530 | 43_6 | 683 | 30 | 0 |
| 3584 | 37 | 183 | 373 | 66_1 | 731 | 30 | 0 |
| 3585 | 37 | 94 | 394 | 69_6_rel | 10250 | 30 | 0 |
| 3586 | 37 | 121 | 502 | 70_14 | 710 | 30 | 0 |
| 3587 | 37 | 177 | 366, 464 | 72_12 | 2283 | 30 | 0 |
| 3588 | 37 | 40 | 553 | 72_4 | 1988 | 30 | 0 |
| 3589 | 37 | 97 | 357 | 74_26_rel | 2988 | 30 | 0 |
| 3590 | 37 | 193 | 398 | 93_3_rel | 33882 | 4 | 1.1e−009 |
| 3591 | 37 | 33 | 339, 552 | 96_rel | 43343 | 5 | 1.39e−012 |
| 3592 | 37 | 87 | 301 | gam11 | 2292 | 30 | 0 |
| 3593 | 37 | 163 | 402 | 43_7 | 1428 | 30 | 0 |
| 3594 | 37 | 223 | 320, 393, 559 | hsa-let-7a | 6720 | 30 | 0 |
| 3595 | 37 | 225 | 332 | hsa-let-7b | 22554 | 30 | 0 |
| 3596 | 37 | 224 | 427 | hsa-let-7c | 15745 | 30 | 0 |
| 3597 | 37 | 45 | 277 | hsa-let-7d | 3472 | 30 | 0 |
| 3598 | 37 | 220 | 475 | hsa-let-7e | 4261 | 30 | 0 |
| 3599 | 37 | 222 | 482, 558 | hsa-let-7f | 773 | 30 | 0 |
| 3600 | 37 | 226 | 395 | hsa-let-7g | 1553 | 30 | 0 |
| 3601 | 37 | 227 | 297 | hsa-let-7i | 2390 | 30 | 0 |
| 3602 | 37 | 231 | 321, 424 | hsa-miR-1 | 554 | 30 | 0 |
| 3603 | 37 | 13 | 397 | hsa-miR-100 | 2943 | 30 | 0 |
| 3604 | 37 | 48 | 483, 529 | hsa-miR-103 | 1723 | 30 | 0 |
| 3605 | 37 | 184 | 423 | hsa-miR-105 | 1246 | 30 | 0 |
| 3606 | 37 | 2 | 399 | hsa-miR-106a | 2056 | 30 | 0 |
| 3607 | 37 | 149 | 392 | hsa-miR-106b | 1342 | 30 | 0 |
| 3608 | 37 | 47 | 555 | hsa-miR-107 | 2508 | 30 | 0 |
| 3609 | 37 | 199 | 387 | hsa-miR-125a | 3713 | 30 | 0 |
| 3610 | 37 | 198 | 313, 551 | hsa-miR-125b | 17181 | 30 | 0 |
| 3611 | 37 | 204 | 419 | hsa-miR-126 | 1563 | 30 | 0 |
| 3612 | 37 | 89 | 419 | hsa-miR-126* | 544 | 30 | 0 |
| 3613 | 37 | 86 | 417 | hsa-miR-130a | 2939 | 30 | 0 |
| 3614 | 37 | 266 | 295, 386 | hsa-miR-133a | 1300 | 30 | 0 |
| 3615 | 37 | 265 | 533 | hsa-miR-133b | 1436 | 30 | 0 |
| 3616 | 37 | 52 | 370, 548 | hsa-miR-138 | 550 | 30 | 0 |
| 3617 | 37 | 64 | 402 | hsa-miR-140 | 682 | 30 | 0 |
| 3618 | 37 | 151 | 472 | hsa-miR-141 | 4124 | 30 | 0 |
| 3619 | 37 | 218 | 527 | hsa-miR-143 | 10003 | 30 | 0 |
| 3620 | 37 | 139 | 281 | hsa-miR-145 | 38044 | 30 | 0 |
| 3621 | 37 | 217 | 466 | hsa-miR-146a | 516 | 30 | 0 |
| 3622 | 37 | 146 | 325 | hsa-miR-147 | 551 | 30 | 0 |
| 3623 | 37 | 190 | 458 | hsa-miR-148a | 576 | 30 | 0 |
| 3624 | 37 | 191 | 289 | hsa-miR-148b | 649 | 30 | 0 |
| 3625 | 37 | 212 | 333 | hsa-miR-149 | 1378 | 30 | 0 |
| 3626 | 37 | 209 | 507 | hsa-miR-150 | 811 | 30 | 0 |
| 3627 | 37 | 34 | 396 | hsa-miR-151 | 1093 | 30 | 0 |
| 3628 | 37 | 192 | 470 | hsa-miR-152 | 1060 | 30 | 0 |
| 3629 | 37 | 263 | 296, 404 | hsa-miR-153 | 554 | 30 | 0 |
| 3630 | 37 | 253 | 499 | hsa-miR-155 | 597 | 30 | 0 |
| 3631 | 37 | 171 | 331 | hsa-miR-15a | 529 | 30 | 0 |
| 3632 | 37 | 172 | 329 | hsa-miR-15b | 693 | 30 | 0 |
| 3633 | 37 | 173 | 360, 468 | hsa-miR-16 | 2746 | 30 | 0 |
| 3634 | 37 | 38 | 467 | hsa-miR-17-3p | 702 | 30 | 0 |
| 3635 | 37 | 75 | 467 | hsa-miR-17-5p | 1496 | 30 | 0 |
| 3636 | 37 | 10 | 371, 461 | hsa-miR-181a | 1328 | 30 | 0 |
| 3637 | 37 | 179 | 356 | hsa-miR-183 | 795 | 30 | 0 |
| 3638 | 37 | 234 | 450 | hsa-miR-185 | 850 | 30 | 0 |
| 3639 | 37 | 88 | 310 | hsa-miR-188 | 607 | 30 | 0 |
| 3640 | 37 | 144 | 346 | hsa-miR-189 | 720 | 30 | 0 |
| 3641 | 37 | 77 | 440 | hsa-miR-191 | 2398 | 30 | 0 |
| 3642 | 37 | 130 | 440 | hsa-miR-191* | 775 | 30 | 0 |
| 3643 | 37 | 17 | 442 | hsa-miR-193a | 3810 | 30 | 0 |
| 3644 | 37 | 16 | 488 | hsa-miR-193b | 564 | 30 | 0 |
| 3645 | 37 | 170 | 327 | hsa-miR-195 | 938 | 30 | 0 |
| 3646 | 37 | 136 | 308 | hsa-miR-198 | 521 | 30 | 0 |
| 3647 | 37 | 92 | 410, 432 | hsa-miR-199a | 4177 | 30 | 0 |
| 3648 | 37 | 162 | 410, 432, 473 | hsa-miR-199a* | 808 | 30 | 0 |
| 3649 | 37 | 251 | 282, 495 | hsa-miR-19b | 1109 | 30 | 0 |
| 3650 | 37 | 152 | 369 | hsa-miR-200a | 761 | 30 | 0 |
| 3651 | 37 | 157 | 411 | hsa-miR-200b | 1385 | 30 | 0 |
| 3652 | 37 | 156 | 446 | hsa-miR-200c | 6496 | 30 | 0 |
| 3653 | 37 | 271 | 334 | hsa-miR-202* | 691 | 30 | 0 |
| 3654 | 37 | 203 | 504 | hsa-miR-205 | 9556 | 30 | 0 |
| 3655 | 37 | 232 | 422 | hsa-miR-206 | 1006 | 30 | 0 |
| 3656 | 37 | 175 | 462 | hsa-miR-21 | 4398 | 30 | 0 |
| 3657 | 37 | 115 | 314 | hsa-miR-210 | 706 | 30 | 0 |
| 3658 | 37 | 154 | 300 | hsa-miR-212 | 595 | 30 | 0 |
| 3659 | 37 | 29 | 439 | hsa-miR-214 | 2821 | 30 | 0 |
| 3660 | 37 | 159 | 494 | hsa-miR-216 | 705 | 30 | 0 |
| 3661 | 37 | 268 | 401, 425 | hsa-miR-218 | 571 | 30 | 0 |
| 3662 | 37 | 18 | 322 | hsa-miR-22 | 3671 | 30 | 0 |
| 3663 | 37 | 50 | 364 | hsa-miR-221 | 6092 | 30 | 0 |
| 3664 | 37 | 49 | 302 | hsa-miR-222 | 9041 | 30 | 0 |
| 3665 | 37 | 248 | 415 | hsa-miR-223 | 505 | 30 | 0 |
| 3666 | 37 | 67 | 336 | hsa-miR-23a | 3251 | 30 | 0 |
| 3667 | 37 | 66 | 306 | hsa-miR-23b | 1385 | 30 | 0 |
| 3668 | 37 | 238 | 346, 416 | hsa-miR-24 | 12695 | 30 | 0 |
| 3669 | 37 | 256 | 441, 487 | hsa-miR-26a | 8333 | 30 | 0 |
| 3670 | 37 | 257 | 409 | hsa-miR-26b | 539 | 30 | 0 |
| 3671 | 37 | 259 | 481 | hsa-miR-27a | 3290 | 30 | 0 |
| 3672 | 37 | 260 | 280 | hsa-miR-27b | 3555 | 30 | 0 |
| 3673 | 37 | 61 | 286 | hsa-miR-296 | 854 | 30 | 0 |
| 3674 | 37 | 167 | 556 | hsa-miR-29a | 655 | 30 | 0 |
| 3675 | 37 | 269 | 362 | hsa-miR-302c* | 546 | 30 | 0 |
| 3676 | 37 | 117 | 287 | hsa-miR-30a-3p | 783 | 30 | 0 |
| 3677 | 37 | 244 | 287 | hsa-miR-30a-5p | 531 | 30 | 0 |
| 3678 | 37 | 242 | 311 | hsa-miR-30b | 833 | 30 | 0 |
| 3679 | 37 | 243 | 299, 316 | hsa-miR-30c | 2389 | 30 | 0 |
| 3680 | 37 | 241 | 550 | hsa-miR-30d | 2668 | 30 | 0 |
| 3681 | 37 | 116 | 431 | hsa-miR-30e-3p | 581 | 30 | 0 |
| 3682 | 37 | 245 | 431 | hsa-miR-30e-5p | 3105 | 30 | 0 |
| 3683 | 37 | 132 | 429 | hsa-miR-31 | 1418 | 30 | 0 |
| 3684 | 37 | 1 | 359 | hsa-miR-320 | 5359 | 30 | 0 |
| 3685 | 37 | 124 | 455 | hsa-miR-323 | 616 | 30 | 0 |
| 3686 | 37 | 91 | 368 | hsa-miR-324-3p | 1163 | 30 | 0 |
| 3687 | 37 | 98 | 368 | hsa-miR-324-5p | 1174 | 30 | 0 |
| 3688 | 37 | 113 | 538 | hsa-miR-328 | 963 | 30 | 0 |
| 3689 | 37 | 141 | 437 | hsa-miR-33 | 1278 | 30 | 0 |
| 3690 | 37 | 126 | 380 | hsa-miR-331 | 869 | 30 | 0 |
| 3691 | 37 | 208 | 477 | hsa-miR-342 | 1726 | 30 | 0 |
| 3692 | 37 | 229 | 292 | hsa-miR-345 | 969 | 30 | 0 |
| 3693 | 37 | 237 | 480 | hsa-miR-34a | 2224 | 30 | 0 |
| 3694 | 37 | 26 | 493 | hsa-miR-363 | 503 | 30 | 0 |
| 3695 | 37 | 160 | 379, 382 | hsa-miR-365 | 837 | 30 | 0 |
| 3696 | 37 | 128 | 294 | hsa-miR-370 | 874 | 30 | 0 |
| 3697 | 37 | 254 | 496 | hsa-miR-374 | 591 | 30 | 0 |
| 3698 | 37 | 105 | 372 | hsa-miR-378 | 504 | 30 | 0 |
| 3699 | 37 | 46 | 298 | hsa-miR-383 | 529 | 30 | 0 |
| 3700 | 37 | 111 | 389 | hsa-miR-422a | 716 | 30 | 0 |
| 3701 | 37 | 112 | 372 | hsa-miR-422b | 1840 | 30 | 0 |
| 3702 | 37 | 51 | 497 | hsa-miR-423 | 2753 | 30 | 0 |
| 3703 | 37 | 69 | 363 | hsa-miR-425-3p | 608 | 30 | 0 |
| 3704 | 37 | 213 | 531 | hsa-miR-432 | 576 | 30 | 0 |
| 3705 | 37 | 236 | 413 | hsa-miR-449 | 2451 | 30 | 0 |
| 3706 | 37 | 274 | 375, 309 | hsa-miR-450 | 553 | 30 | 0 |
| 3707 | 37 | 252 | 405 | hsa-miR-452 | 1039 | 30 | 0 |
| 3708 | 37 | 138 | 547 | hsa-miR-485-3p | 519 | 30 | 0 |
| 3709 | 37 | 63 | 557 | hsa-miR-491 | 2864 | 30 | 0 |
| 3710 | 37 | 54 | 293 | hsa-miR-492 | 844 | 30 | 0 |
| 3711 | 37 | 267 | 354 | hsa-miR-493-5p | 660 | 30 | 0 |
| 3712 | 37 | 214 | 376 | hsa-miR-494 | 7459 | 30 | 0 |
| 3713 | 37 | 3 | 543 | hsa-miR-495 | 695 | 30 | 0 |
| 3714 | 37 | 83 | 312 | hsa-miR-497 | 2702 | 30 | 0 |
| 3715 | 37 | 25 | 414 | hsa-miR-501 | 565 | 30 | 0 |
| 3716 | 37 | 43 | 418 | hsa-miR-504 | 1024 | 30 | 0 |
| 3717 | 37 | 258 | 428, 443 | hsa-miR-513 | 1422 | 30 | 0 |
| 3718 | 37 | 73 | 503 | hsa-miR-518b | 707 | 30 | 0 |
| 3719 | 37 | 210 | 400 | hsa-miR-518c* | 849 | 30 | 0 |
| 3720 | 37 | 262 | 523 | hsa-miR-519e* | 689 | 30 | 0 |
| 3721 | 37 | 206 | 513 | hsa-miR-520d* | 661 | 30 | 0 |
| 3722 | 37 | 100 | 524 | hsa-miR-524* | 664 | 30 | 0 |
| 3723 | 37 | 118 | 342 | hsa-miR-525* | 563 | 30 | 0 |
| 3724 | 37 | 110 | 511, 512, 514 | hsa-miR-527 | 551 | 30 | 0 |
| 3725 | 37 | 148 | 283 | hsa-miR-557 | 549 | 30 | 0 |
| 3726 | 37 | 135 | 315 | hsa-miR-565 | 4041 | 30 | 0 |
| 3727 | 37 | 60 | 479 | hsa-miR-638 | 18669 | 30 | 0 |
| 3728 | 37 | 230 | 474, 484, 536 | hsa-miR-7 | 601 | 30 | 0 |
| 3729 | 37 | 182 | 304, 317 | hsa-miR-92 | 2601 | 30 | 0 |
| 3730 | 37 | 6 | 471 | hsa-miR-93 | 1501 | 30 | 0 |
| 3731 | 37 | 14 | 318 | hsa-miR-99a | 13455 | 30 | 0 |
| 3732 | 37 | 80 | 509 | hsa-miR-99b | 1634 | 30 | 0 |
| 3733 | 37 | 131 | 430 | 6_11 | 741 | 30 | 0 |
| 3734 | 38 | 196 | 508 | 15_rel | 48717 | 7 | 1.35e−017 |
| 3735 | 38 | 219 | 497 | 27_3 | 1957 | 7 | 1.01e−017 |
| 3736 | 38 | 99 | 348, 384, 434 | 43_35 | 603 | 7 | 1.01e−017 |
| 3737 | 38 | 129 | 548 | 43_39 | 1169 | 7 | 1.25e−018 |
| 3738 | 38 | 27 | 313 | 43_40 | 529 | 7 | 1.77e−017 |
| 3739 | 38 | 183 | 373 | 66_1 | 544 | 7 | 1.96e−018 |
| 3740 | 38 | 94 | 394 | 69_6_rel | 11039 | 7 | 1.68e−017 |
| 3741 | 38 | 177 | 366, 464 | 72_12 | 1020 | 7 | 3.12e−018 |
| 3742 | 38 | 40 | 553 | 72_4 | 895 | 7 | 5.9e−017 |
| 3743 | 38 | 97 | 357 | 74_26_rel | 4452 | 7 | 2.04e−017 |
| 3744 | 38 | 87 | 301 | gam11 | 6859 | 7 | 2.73e−016 |
| 3745 | 38 | 163 | 402 | 43_7 | 743 | 7 | 1.19e−018 |
| 3746 | 38 | 223 | 320, 393, 559 | hsa-let-7a | 6130 | 7 | 6.27e−017 |
| 3747 | 38 | 225 | 332 | hsa-let-7b | 17358 | 7 | 7.06e−015 |
| 3748 | 38 | 224 | 427 | hsa-let-7c | 15082 | 7 | 5.71e−019 |
| 3749 | 38 | 45 | 277 | hsa-let-7d | 2375 | 7 | 9.32e−017 |
| 3750 | 38 | 220 | 475 | hsa-let-7e | 3084 | 7 | 1.05e−018 |
| 3751 | 38 | 222 | 482, 558 | hsa-let-7f | 613 | 7 | 2.43e−017 |
| 3752 | 38 | 226 | 395 | hsa-let-7g | 1582 | 7 | 6.21e−018 |
| 3753 | 38 | 227 | 297 | hsa-let-7i | 1630 | 7 | 1.13e−017 |
| 3754 | 38 | 13 | 397 | hsa-miR-100 | 2229 | 7 | 1.1e−016 |
| 3755 | 38 | 48 | 483, 529 | hsa-miR-103 | 860 | 7 | 1.66e−017 |
| 3756 | 38 | 184 | 423 | hsa-miR-105 | 657 | 7 | 2.14e−017 |
| 3757 | 38 | 2 | 399 | hsa-miR-106a | 855 | 7 | 2.8e−017 |
| 3758 | 38 | 149 | 392 | hsa-miR-106b | 584 | 7 | 4.05e−017 |
| 3759 | 38 | 47 | 555 | hsa-miR-107 | 979 | 7 | 2.17e−018 |
| 3760 | 38 | 199 | 387 | hsa-miR-125a | 2080 | 7 | 2.12e−017 |
| 3761 | 38 | 198 | 313, 551 | hsa-miR-125b | 11329 | 7 | 6.8e−016 |
| 3762 | 38 | 204 | 419 | hsa-miR-126 | 603 | 7 | 8.29e−018 |
| 3763 | 38 | 86 | 417 | hsa-miR-130a | 1522 | 7 | 2.8e−018 |
| 3764 | 38 | 266 | 295, 386 | hsa-miR-133a | 558 | 7 | 3.38e−017 |
| 3765 | 38 | 265 | 533 | hsa-miR-133b | 1011 | 7 | 6.98e−017 |
| 3766 | 38 | 151 | 472 | hsa-miR-141 | 2021 | 7 | 1.12e−016 |
| 3767 | 38 | 218 | 527 | hsa-miR-143 | 9263 | 7 | 5.12e−013 |
| 3768 | 38 | 139 | 281 | hsa-miR-145 | 44181 | 7 | 1.03e−017 |
| 3769 | 38 | 146 | 325 | hsa-miR-147 | 541 | 7 | 2.58e−019 |
| 3770 | 38 | 191 | 289 | hsa-miR-148b | 501 | 7 | 2.75e−018 |
| 3771 | 38 | 212 | 333 | hsa-miR-149 | 1315 | 7 | 6.06e−017 |
| 3772 | 38 | 209 | 507 | hsa-miR-150 | 562 | 7 | 3.9e−018 |
| 3773 | 38 | 34 | 396 | hsa-miR-151 | 702 | 7 | 4e−017 |
| 3774 | 38 | 192 | 470 | hsa-miR-152 | 632 | 7 | 3.32e−018 |
| 3775 | 38 | 263 | 296, 404 | hsa-miR-153 | 538 | 7 | 2.63e−017 |
| 3776 | 38 | 173 | 360, 468 | hsa-miR-16 | 1445 | 7 | 2.02e−016 |
| 3777 | 38 | 75 | 467 | hsa-miR-17-5p | 621 | 7 | 1.02e−017 |
| 3778 | 38 | 10 | 371, 461 | hsa-miR-181a | 569 | 7 | 3.44e−018 |
| 3779 | 38 | 179 | 356 | hsa-miR-183 | 859 | 7 | 1.21e−017 |
| 3780 | 38 | 77 | 440 | hsa-miR-191 | 1005 | 7 | 4.14e−017 |
| 3781 | 38 | 130 | 440 | hsa-miR-191* | 663 | 7 | 4.07e−017 |
| 3782 | 38 | 17 | 442 | hsa-miR-193a | 1383 | 7 | 1.96e−016 |
| 3783 | 38 | 16 | 488 | hsa-miR-193b | 716 | 7 | 2.92e−017 |
| 3784 | 38 | 92 | 410, 432 | hsa-miR-199a | 1866 | 7 | 9.17e−017 |
| 3785 | 38 | 152 | 369 | hsa-miR-200a | 553 | 7 | 1.81e−017 |
| 3786 | 38 | 157 | 411 | hsa-miR-200b | 858 | 7 | 5.7e−018 |
| 3787 | 38 | 156 | 446 | hsa-miR-200c | 4662 | 7 | 3.19e−017 |
| 3788 | 38 | 203 | 504 | hsa-miR-205 | 10121 | 7 | 9.32e−017 |
| 3789 | 38 | 232 | 422 | hsa-miR-206 | 1121 | 7 | 9.6e−017 |
| 3790 | 38 | 175 | 462 | hsa-miR-21 | 3632 | 7 | 6.31e−017 |
| 3791 | 38 | 154 | 300 | hsa-miR-212 | 609 | 7 | 2.11e−018 |
| 3792 | 38 | 29 | 439 | hsa-miR-214 | 1549 | 7 | 1.54e−018 |
| 3793 | 38 | 18 | 322 | hsa-miR-22 | 1516 | 7 | 4.46e−017 |
| 3794 | 38 | 50 | 364 | hsa-miR-221 | 2700 | 7 | 2.64e−016 |
| 3795 | 38 | 49 | 302 | hsa-miR-222 | 4412 | 7 | 4.66e−017 |
| 3796 | 38 | 67 | 336 | hsa-miR-23a | 1231 | 7 | 1.08e−016 |
| 3797 | 38 | 66 | 306 | hsa-miR-23b | 3223 | 7 | 1.25e−013 |
| 3798 | 38 | 238 | 346, 416 | hsa-miR-24 | 5256 | 7 | 5.11e−016 |
| 3799 | 38 | 90 | 388 | hsa-miR-25 | 509 | 7 | 3.35e−018 |
| 3800 | 38 | 256 | 441, 487 | hsa-miR-26a | 4628 | 7 | 2.59e−017 |
| 3801 | 38 | 257 | 409 | hsa-miR-26b | 745 | 7 | 2.04e−018 |
| 3802 | 38 | 259 | 481 | hsa-miR-27a | 1219 | 7 | 3.02e−016 |
| 3803 | 38 | 260 | 280 | hsa-miR-27b | 1908 | 7 | 7.77e−016 |
| 3804 | 38 | 61 | 286 | hsa-miR-296 | 1183 | 7 | 2.36e−014 |
| 3805 | 38 | 167 | 556 | hsa-miR-29a | 1178 | 7 | 2.48e−014 |
| 3806 | 38 | 243 | 299, 316 | hsa-miR-30c | 1181 | 7 | 1.69e−016 |
| 3807 | 38 | 241 | 550 | hsa-miR-30d | 1557 | 7 | 1.01e−017 |
| 3808 | 38 | 245 | 431 | hsa-miR-30e-5p | 2053 | 7 | 3.57e−017 |
| 3809 | 38 | 132 | 429 | hsa-miR-31 | 1015 | 7 | 3.71e−018 |
| 3810 | 38 | 1 | 359 | hsa-miR-320 | 4128 | 7 | 1.24e−016 |
| 3811 | 38 | 124 | 455 | hsa-miR-323 | 515 | 7 | 5.45e−015 |
| 3812 | 38 | 91 | 368 | hsa-miR-324-3p | 872 | 7 | 1.16e−018 |
| 3813 | 38 | 98 | 368 | hsa-miR-324-5p | 1029 | 7 | 1.33e−017 |
| 3814 | 38 | 113 | 538 | hsa-miR-328 | 674 | 7 | 2.39e−017 |
| 3815 | 38 | 141 | 437 | hsa-miR-33 | 1103 | 7 | 2.01e−018 |
| 3816 | 38 | 208 | 477 | hsa-miR-342 | 894 | 7 | 4.69e−019 |
| 3817 | 38 | 229 | 292 | hsa-miR-345 | 554 | 7 | 5.25e−018 |
| 3818 | 38 | 237 | 480 | hsa-miR-34a | 676 | 7 | 2.92e−017 |
| 3819 | 38 | 255 | 444 | hsa-miR-361 | 535 | 7 | 4.59e−018 |
| 3820 | 38 | 160 | 379, 382 | hsa-miR-365 | 561 | 7 | 7.27e−020 |
| 3821 | 38 | 128 | 294 | hsa-miR-370 | 532 | 7 | 1.54e−016 |
| 3822 | 38 | 254 | 496 | hsa-miR-374 | 513 | 7 | 4.55e−018 |
| 3823 | 38 | 111 | 389 | hsa-miR-422a | 602 | 7 | 6.15e−018 |
| 3824 | 38 | 112 | 372 | hsa-miR-422b | 1250 | 7 | 8.63e−017 |
| 3825 | 38 | 51 | 497 | hsa-miR-423 | 2280 | 7 | 3.98e−018 |
| 3826 | 38 | 69 | 363 | hsa-miR-425-3p | 530 | 7 | 7.32e−018 |
| 3827 | 38 | 213 | 531 | hsa-miR-432 | 551 | 7 | 2.41e−017 |
| 3828 | 38 | 236 | 413 | hsa-miR-449 | 3045 | 7 | 2.98e−018 |
| 3829 | 38 | 274 | 375, 309 | hsa-miR-450 | 616 | 7 | 3.43e−018 |
| 3830 | 38 | 252 | 405 | hsa-miR-452 | 946 | 7 | 3.52e−018 |
| 3831 | 38 | 138 | 547 | hsa-miR-485-3p | 517 | 7 | 4.16e−017 |
| 3832 | 38 | 63 | 557 | hsa-miR-491 | 2602 | 7 | 3.67e−018 |
| 3833 | 38 | 54 | 293 | hsa-miR-492 | 691 | 7 | 3.45e−018 |
| 3834 | 38 | 267 | 354 | hsa-miR-493-5p | 681 | 7 | 7.16e−017 |
| 3835 | 38 | 214 | 376 | hsa-miR-494 | 13900 | 7 | 5.47e−017 |
| 3836 | 38 | 3 | 543 | hsa-miR-495 | 722 | 7 | 1.16e−017 |
| 3837 | 38 | 83 | 312 | hsa-miR-497 | 1437 | 7 | 3.82e−018 |
| 3838 | 38 | 43 | 418 | hsa-miR-504 | 702 | 7 | 3.8e−018 |
| 3839 | 38 | 258 | 428, 443 | hsa-miR-513 | 1324 | 7 | 2.03e−018 |
| 3840 | 38 | 73 | 503 | hsa-miR-518b | 643 | 7 | 2.64e−017 |
| 3841 | 38 | 210 | 400 | hsa-miR-518c* | 580 | 7 | 5.86e−018 |
| 3842 | 38 | 262 | 523 | hsa-miR-519e* | 589 | 7 | 5.37e−019 |
| 3843 | 38 | 206 | 513 | hsa-miR-520d* | 518 | 7 | 5.89e−017 |
| 3844 | 38 | 100 | 524 | hsa-miR-524* | 531 | 7 | 1.68e−017 |
| 3845 | 38 | 118 | 342 | hsa-miR-525* | 711 | 7 | 1.09e−016 |
| 3846 | 38 | 135 | 315 | hsa-miR-565 | 2207 | 7 | 8.96e−018 |
| 3847 | 38 | 60 | 479 | hsa-miR-638 | 17139 | 7 | 3.48e−018 |
| 3848 | 38 | 230 | 474, 484, 536 | hsa-miR-7 | 571 | 7 | 1.33e−016 |
| 3849 | 38 | 182 | 304, 317 | hsa-miR-92 | 1202 | 7 | 8.35e−018 |
| 3850 | 38 | 6 | 471 | hsa-miR-93 | 712 | 7 | 1.64e−017 |
| 3851 | 38 | 14 | 318 | hsa-miR-99a | 8571 | 7 | 1.1e−015 |
| 3852 | 38 | 80 | 509 | hsa-miR-99b | 825 | 7 | 6.6e−019 |
| 3853 | 38 | 131 | 430 | 6_11 | 582 | 7 | 4.54e−018 |
| 3854 | 39 | 127 | 361 | 11_1 | 1121 | 5 | 3.21e−014 |
| 3855 | 39 | 196 | 508 | 15_rel | 60307 | 5 | 5.48e−014 |
| 3856 | 39 | 197 | 444 | 25_6 | 708 | 5 | 5.12e−014 |
| 3857 | 39 | 219 | 497 | 27_3 | 3628 | 5 | 4.82e−014 |
| 3858 | 39 | 122 | 281 | 43_11 | 765 | 5 | 2.13e−015 |
| 3859 | 39 | 7 | 337 | 43_21 | 756 | 5 | 7.61e−014 |
| 3860 | 39 | 78 | 318 | 43_24 | 558 | 5 | 7.22e−016 |
| 3861 | 39 | 42 | 449 | 43_3 | 1073 | 5 | 1.92e−014 |
| 3862 | 39 | 101 | 475 | 43_30 | 749 | 5 | 1.21e−014 |
| 3863 | 39 | 99 | 348, 384, 434 | 43_35 | 922 | 5 | 1.18e−014 |
| 3864 | 39 | 129 | 548 | 43_39 | 2821 | 5 | 1.99e−015 |
| 3865 | 39 | 211 | 353 | 43_44 | 750 | 5 | 5.61e−014 |
| 3866 | 39 | 114 | 530 | 43_6 | 1017 | 5 | 1.06e−014 |
| 3867 | 39 | 183 | 373 | 66_1 | 1373 | 5 | 3.18e−014 |
| 3868 | 39 | 94 | 394 | 69_6_rel | 25322 | 5 | 1.67e−014 |
| 3869 | 39 | 121 | 502 | 70_14 | 1223 | 5 | 9.79e−014 |
| 3870 | 39 | 177 | 366, 464 | 72_12 | 2170 | 5 | 3.04e−013 |
| 3871 | 39 | 40 | 553 | 72_4 | 3258 | 5 | 5.33e−014 |
| 3872 | 39 | 97 | 357 | 74_26_rel | 6144 | 5 | 5.38e−015 |
| 3873 | 39 | 163 | 402 | 43_7 | 1982 | 5 | 2.2e−013 |
| 3874 | 39 | 223 | 320, 393, 559 | hsa-let-7a | 7573 | 5 | 1.3e−012 |
| 3875 | 39 | 225 | 332 | hsa-let-7b | 38974 | 5 | 3.5e−014 |
| 3876 | 39 | 224 | 427 | hsa-let-7c | 20447 | 5 | 3.9e−013 |
| 3877 | 39 | 45 | 277 | hsa-let-7d | 3924 | 5 | 2e−014 |
| 3878 | 39 | 220 | 475 | hsa-let-7e | 5026 | 5 | 1.11e−015 |
| 3879 | 39 | 222 | 482, 558 | hsa-let-7f | 1027 | 5 | 9.15e−015 |
| 3880 | 39 | 226 | 395 | hsa-let-7g | 2698 | 5 | 2.84e−014 |
| 3881 | 39 | 227 | 297 | hsa-let-7i | 2897 | 5 | 7.38e−015 |
| 3882 | 39 | 231 | 321, 424 | hsa-miR-1 | 971 | 5 | 4.11e−014 |
| 3883 | 39 | 13 | 397 | hsa-miR-100 | 4766 | 5 | 5.37e−014 |
| 3884 | 39 | 48 | 483, 529 | hsa-miR-103 | 1360 | 5 | 1.69e−010 |
| 3885 | 39 | 184 | 423 | hsa-miR-105 | 1634 | 5 | 7.52e−015 |
| 3886 | 39 | 2 | 399 | hsa-miR-106a | 2191 | 5 | 2.94e−014 |
| 3887 | 39 | 149 | 392 | hsa-miR-106b | 1533 | 5 | 2.52e−014 |
| 3888 | 39 | 47 | 555 | hsa-miR-107 | 2776 | 5 | 1.81e−014 |
| 3889 | 39 | 235 | 358 | hsa-miR-122a | 605 | 5 | 2.41e−014 |
| 3890 | 39 | 199 | 387 | hsa-miR-125a | 4675 | 5 | 2.67e−015 |
| 3891 | 39 | 198 | 313, 551 | hsa-miR-125b | 25367 | 5 | 4.54e−014 |
| 3892 | 39 | 204 | 419 | hsa-miR-126 | 1380 | 5 | 2.94e−012 |
| 3893 | 39 | 89 | 419 | hsa-miR-126* | 946 | 5 | 5.2e−015 |
| 3894 | 39 | 86 | 417 | hsa-miR-130a | 3557 | 5 | 3.02e−015 |
| 3895 | 39 | 85 | 328 | hsa-miR-130b | 855 | 5 | 9.85e−014 |
| 3896 | 39 | 153 | 445 | hsa-miR-132 | 760 | 5 | 5.51e−014 |
| 3897 | 39 | 266 | 295, 386 | hsa-miR-133a | 2058 | 5 | 1.31e−012 |
| 3898 | 39 | 265 | 533 | hsa-miR-133b | 1842 | 5 | 9.21e−013 |
| 3899 | 39 | 181 | 324, 554 | hsa-miR-135a | 1064 | 5 | 2.18e−013 |
| 3900 | 39 | 180 | 486 | hsa-miR-135b | 915 | 5 | 1.6e−014 |
| 3901 | 39 | 37 | 391 | hsa-miR-136 | 573 | 5 | 5.22e−011 |
| 3902 | 39 | 52 | 370, 548 | hsa-miR-138 | 1127 | 5 | 4.76e−014 |
| 3903 | 39 | 64 | 402 | hsa-miR-140 | 1468 | 5 | 3.18e−014 |
| 3904 | 39 | 151 | 472 | hsa-miR-141 | 3598 | 5 | 2.53e−013 |
| 3905 | 39 | 247 | 278 | hsa-miR-142-3p | 571 | 5 | 1.82e−014 |
| 3906 | 39 | 218 | 527 | hsa-miR-143 | 9891 | 5 | 1.15e−009 |
| 3907 | 39 | 139 | 281 | hsa-miR-145 | 60369 | 5 | 6.83e−014 |
| 3908 | 39 | 217 | 466 | hsa-miR-146a | 608 | 5 | 1.61e−014 |
| 3909 | 39 | 146 | 325 | hsa-miR-147 | 968 | 5 | 1.37e−014 |
| 3910 | 39 | 190 | 458 | hsa-miR-148a | 651 | 5 | 3.48e−014 |
| 3911 | 39 | 191 | 289 | hsa-miR-148b | 836 | 5 | 1.74e−014 |
| 3912 | 39 | 212 | 333 | hsa-miR-149 | 1844 | 5 | 2.62e−014 |
| 3913 | 39 | 209 | 507 | hsa-miR-150 | 713 | 5 | 3.57e−013 |
| 3914 | 39 | 34 | 396 | hsa-miR-151 | 1134 | 5 | 7.96e−015 |
| 3915 | 39 | 192 | 470 | hsa-miR-152 | 1221 | 5 | 4.14e−014 |
| 3916 | 39 | 263 | 296, 404 | hsa-miR-153 | 857 | 5 | 2.89e−014 |
| 3917 | 39 | 253 | 499 | hsa-miR-155 | 785 | 5 | 2.46e−014 |
| 3918 | 39 | 171 | 331 | hsa-miR-15a | 767 | 5 | 7.68e−014 |
| 3919 | 39 | 172 | 329 | hsa-miR-15b | 961 | 5 | 1.6e−014 |
| 3920 | 39 | 173 | 360, 468 | hsa-miR-16 | 2348 | 5 | 2.38e−012 |
| 3921 | 39 | 38 | 467 | hsa-miR-17-3p | 827 | 5 | 7.53e−016 |
| 3922 | 39 | 75 | 467 | hsa-miR-17-5p | 1491 | 5 | 4.78e−014 |
| 3923 | 39 | 10 | 371, 461 | hsa-miR-181a | 1489 | 5 | 1.23e−013 |
| 3924 | 39 | 32 | 461 | hsa-miR-181a* | 557 | 5 | 1.74e−013 |
| 3925 | 39 | 11 | 456, 540 | hsa-miR-181b | 594 | 5 | 3.27e−016 |
| 3926 | 39 | 179 | 356 | hsa-miR-183 | 2067 | 5 | 6.45e−014 |
| 3927 | 39 | 233 | 305 | hsa-miR-184 | 712 | 5 | 1.29e−013 |
| 3928 | 39 | 234 | 450 | hsa-miR-185 | 1017 | 5 | 2.2e−015 |
| 3929 | 39 | 205 | 367 | hsa-miR-187 | 708 | 5 | 1.84e−015 |
| 3930 | 39 | 88 | 310 | hsa-miR-188 | 1122 | 5 | 1.32e−013 |
| 3931 | 39 | 144 | 346 | hsa-miR-189 | 1039 | 5 | 1.36e−015 |
| 3932 | 39 | 228 | 435 | hsa-miR-190 | 602 | 5 | 7.59e−014 |
| 3933 | 39 | 77 | 440 | hsa-miR-191 | 2920 | 5 | 5.51e−014 |
| 3934 | 39 | 130 | 440 | hsa-miR-191* | 915 | 5 | 7.18e−013 |
| 3935 | 39 | 109 | 374 | hsa-miR-192 | 630 | 5 | 2.85e−014 |
| 3936 | 39 | 17 | 442 | hsa-miR-193a | 4476 | 5 | 7.32e−014 |
| 3937 | 39 | 170 | 327 | hsa-miR-195 | 1129 | 5 | 1.52e−012 |
| 3938 | 39 | 136 | 308 | hsa-miR-198 | 1011 | 5 | 3.76e−014 |
| 3939 | 39 | 92 | 410, 432 | hsa-miR-199a | 5623 | 5 | 4.63e−013 |
| 3940 | 39 | 162 | 410, 432, 473 | hsa-miR-199a* | 899 | 5 | 8.45e−013 |
| 3941 | 39 | 93 | 473 | hsa-miR-199b | 818 | 5 | 3.1e−014 |
| 3942 | 39 | 251 | 282, 495 | hsa-miR-19b | 1278 | 5 | 7.33e−014 |
| 3943 | 39 | 152 | 369 | hsa-miR-200a | 541 | 5 | 5.27e−011 |
| 3944 | 39 | 157 | 411 | hsa-miR-200b | 1682 | 5 | 1.87e−012 |
| 3945 | 39 | 156 | 446 | hsa-miR-200c | 5305 | 5 | 5.92e−014 |
| 3946 | 39 | 271 | 334 | hsa-miR-202* | 1295 | 5 | 2.69e−014 |
| 3947 | 39 | 203 | 504 | hsa-miR-205 | 10130 | 5 | 4.85e−012 |
| 3948 | 39 | 232 | 422 | hsa-miR-206 | 1592 | 5 | 1e−013 |
| 3949 | 39 | 65 | 510 | hsa-miR-208 | 614 | 5 | 7e−014 |
| 3950 | 39 | 175 | 462 | hsa-miR-21 | 3033 | 5 | 6.66e−012 |
| 3951 | 39 | 115 | 314 | hsa-miR-210 | 984 | 5 | 7.58e−014 |
| 3952 | 39 | 154 | 300 | hsa-miR-212 | 1126 | 5 | 9.39e−014 |
| 3953 | 39 | 29 | 439 | hsa-miR-214 | 4319 | 5 | 3.18e−013 |
| 3954 | 39 | 159 | 494 | hsa-miR-216 | 1250 | 5 | 1.4e−014 |
| 3955 | 39 | 268 | 401, 425 | hsa-miR-218 | 1086 | 5 | 3.07e−014 |
| 3956 | 39 | 18 | 322 | hsa-miR-22 | 3160 | 5 | 4.96e−013 |
| 3957 | 39 | 50 | 364 | hsa-miR-221 | 7662 | 5 | 7.54e−014 |
| 3958 | 39 | 49 | 302 | hsa-miR-222 | 15166 | 5 | 1.62e−012 |
| 3959 | 39 | 248 | 415 | hsa-miR-223 | 845 | 5 | 5.27e−014 |
| 3960 | 39 | 67 | 336 | hsa-miR-23a | 3370 | 5 | 8.71e−013 |
| 3961 | 39 | 238 | 346, 416 | hsa-miR-24 | 22242 | 5 | 3.18e−015 |
| 3962 | 39 | 256 | 441, 487 | hsa-miR-26a | 10070 | 5 | 4.83e−013 |
| 3963 | 39 | 257 | 409 | hsa-miR-26b | 789 | 5 | 8.02e−011 |
| 3964 | 39 | 259 | 481 | hsa-miR-27a | 3901 | 5 | 3.72e−013 |
| 3965 | 39 | 260 | 280 | hsa-miR-27b | 4087 | 5 | 8.1e−013 |
| 3966 | 39 | 19 | 457 | hsa-miR-28 | 591 | 5 | 6.65e−014 |
| 3967 | 39 | 61 | 286 | hsa-miR-296 | 617 | 5 | 4.02e−010 |
| 3968 | 39 | 41 | 345 | hsa-miR-302b* | 522 | 5 | 1.01e−014 |
| 3969 | 39 | 269 | 362 | hsa-miR-302c* | 992 | 5 | 8.07e−015 |
| 3970 | 39 | 117 | 287 | hsa-miR-30a-3p | 1115 | 5 | 1.02e−014 |
| 3971 | 39 | 242 | 311 | hsa-miR-30b | 832 | 5 | 2.28e−012 |
| 3972 | 39 | 243 | 299, 316 | hsa-miR-30c | 2190 | 5 | 1.43e−012 |
| 3973 | 39 | 241 | 550 | hsa-miR-30d | 2137 | 5 | 2.79e−014 |
| 3974 | 39 | 116 | 431 | hsa-miR-30e-3p | 978 | 5 | 3.07e−014 |
| 3975 | 39 | 245 | 431 | hsa-miR-30e-5p | 2435 | 5 | 3.51e−013 |
| 3976 | 39 | 132 | 429 | hsa-miR-31 | 1681 | 5 | 2.38e−014 |
| 3977 | 39 | 1 | 359 | hsa-miR-320 | 7070 | 5 | 2.08e−013 |
| 3978 | 39 | 124 | 455 | hsa-miR-323 | 909 | 5 | 2.39e−014 |
| 3979 | 39 | 91 | 368 | hsa-miR-324-3p | 1456 | 5 | 5.33e−014 |
| 3980 | 39 | 98 | 368 | hsa-miR-324-5p | 1498 | 5 | 7.24e−015 |
| 3981 | 39 | 95 | 279 | hsa-miR-325 | 671 | 5 | 4.01e−014 |
| 3982 | 39 | 113 | 538 | hsa-miR-328 | 1706 | 5 | 1.14e−014 |
| 3983 | 39 | 28 | 544, 545 | hsa-miR-329 | 730 | 5 | 5.95e−014 |
| 3984 | 39 | 141 | 437 | hsa-miR-33 | 2187 | 5 | 3.34e−015 |
| 3985 | 39 | 126 | 380 | hsa-miR-331 | 942 | 5 | 2.19e−014 |
| 3986 | 39 | 185 | 492 | hsa-miR-335 | 872 | 5 | 7.27e−015 |
| 3987 | 39 | 194 | 337 | hsa-miR-338 | 600 | 5 | 2.1e−014 |
| 3988 | 39 | 200 | 448 | hsa-miR-339 | 581 | 5 | 2.74e−014 |
| 3989 | 39 | 208 | 477 | hsa-miR-342 | 2150 | 5 | 4.53e−014 |
| 3990 | 39 | 229 | 292 | hsa-miR-345 | 1637 | 5 | 6.41e−014 |
| 3991 | 39 | 237 | 480 | hsa-miR-34a | 2414 | 5 | 2.37e−015 |
| 3992 | 39 | 57 | 498 | hsa-miR-34c | 593 | 5 | 2.62e−015 |
| 3993 | 39 | 24 | 408 | hsa-miR-362 | 544 | 5 | 1.16e−014 |
| 3994 | 39 | 160 | 379, 382 | hsa-miR-365 | 1354 | 5 | 1.18e−014 |
| 3995 | 39 | 31 | 560 | hsa-miR-368 | 608 | 5 | 3.46e−015 |
| 3996 | 39 | 128 | 294 | hsa-miR-370 | 1776 | 5 | 1.04e−013 |
| 3997 | 39 | 254 | 496 | hsa-miR-374 | 992 | 5 | 3.66e−015 |
| 3998 | 39 | 105 | 372 | hsa-miR-378 | 849 | 5 | 1.99e−014 |
| 3999 | 39 | 239 | 349 | hsa-miR-379 | 600 | 5 | 5.43e−014 |
| 4000 | 39 | 178 | 352 | hsa-miR-381 | 792 | 5 | 1.81e−014 |
| 4001 | 39 | 46 | 298 | hsa-miR-383 | 976 | 5 | 2.1e−014 |
| 4002 | 39 | 72 | 490 | hsa-miR-384 | 604 | 5 | 2.54e−013 |
| 4003 | 39 | 111 | 389 | hsa-miR-422a | 995 | 5 | 4.89e−014 |
| 4004 | 39 | 112 | 372 | hsa-miR-422b | 2450 | 5 | 9.57e−014 |
| 4005 | 39 | 51 | 497 | hsa-miR-423 | 4458 | 5 | 8.79e−014 |
| 4006 | 39 | 82 | 377 | hsa-miR-424 | 716 | 5 | 3.38e−016 |
| 4007 | 39 | 69 | 363 | hsa-miR-425-3p | 847 | 5 | 3.74e−013 |
| 4008 | 39 | 158 | 378 | hsa-miR-429 | 561 | 5 | 1.11e−014 |
| 4009 | 39 | 213 | 531 | hsa-miR-432 | 920 | 5 | 6.21e−014 |
| 4010 | 39 | 264 | 541 | hsa-miR-448 | 667 | 5 | 3.85e−014 |
| 4011 | 39 | 236 | 413 | hsa-miR-449 | 5102 | 5 | 2.24e−014 |
| 4012 | 39 | 274 | 375, 309 | hsa-miR-450 | 917 | 5 | 1.16e−013 |
| 4013 | 39 | 252 | 405 | hsa-miR-452 | 1927 | 5 | 1.79e−013 |
| 4014 | 39 | 138 | 547 | hsa-miR-485-3p | 789 | 5 | 1e−013 |
| 4015 | 39 | 62 | 485 | hsa-miR-489 | 867 | 5 | 4.06e−014 |
| 4016 | 39 | 76 | 535 | hsa-miR-490 | 632 | 5 | 3.52e−014 |
| 4017 | 39 | 63 | 557 | hsa-miR-491 | 6374 | 5 | 1.48e−013 |
| 4018 | 39 | 54 | 293 | hsa-miR-492 | 1468 | 5 | 8.88e−015 |
| 4019 | 39 | 267 | 354 | hsa-miR-493-5p | 975 | 5 | 3.38e−014 |
| 4020 | 39 | 214 | 376 | hsa-miR-494 | 17791 | 5 | 4.42e−015 |
| 4021 | 39 | 3 | 543 | hsa-miR-495 | 1187 | 5 | 1.84e−013 |
| 4022 | 39 | 83 | 312 | hsa-miR-497 | 2488 | 5 | 1.59e−013 |
| 4023 | 39 | 25 | 414 | hsa-miR-501 | 929 | 5 | 4.88e−014 |
| 4024 | 39 | 68 | 288 | hsa-miR-502 | 860 | 5 | 2.5e−014 |
| 4025 | 39 | 174 | 534 | hsa-miR-503 | 638 | 5 | 2.74e−014 |
| 4026 | 39 | 43 | 418 | hsa-miR-504 | 1951 | 5 | 1.99e−013 |
| 4027 | 39 | 81 | 454 | hsa-miR-512-5p | 646 | 5 | 2.42e−014 |
| 4028 | 39 | 258 | 428, 443 | hsa-miR-513 | 3143 | 5 | 1.3e−014 |
| 4029 | 39 | 120 | 522 | hsa-miR-515-3p | 670 | 5 | 1.29e−014 |
| 4030 | 39 | 261 | 522 | hsa-miR-515-5p | 648 | 5 | 5.26e−014 |
| 4031 | 39 | 70 | 518 | hsa-miR-516-5p | 629 | 5 | 1.97e−014 |
| 4032 | 39 | 96 | 365, 478, 516 | hsa-miR-517* | 561 | 5 | 3.24e−014 |
| 4033 | 39 | 73 | 503 | hsa-miR-518b | 1076 | 5 | 3.59e−014 |
| 4034 | 39 | 210 | 400 | hsa-miR-518c* | 1765 | 5 | 1.13e−013 |
| 4035 | 39 | 74 | 505 | hsa-miR-518d | 602 | 5 | 4.88e−014 |
| 4036 | 39 | 262 | 523 | hsa-miR-519e* | 1260 | 5 | 1.73e−014 |
| 4037 | 39 | 102 | 344 | hsa-miR-520a* | 803 | 5 | 2.13e−015 |
| 4038 | 39 | 206 | 513 | hsa-miR-520d* | 1470 | 5 | 4.15e−014 |
| 4039 | 39 | 100 | 524 | hsa-miR-524* | 1216 | 5 | 3.08e−014 |
| 4040 | 39 | 118 | 342 | hsa-miR-525* | 1028 | 5 | 5.02e−013 |
| 4041 | 39 | 110 | 511, 512, 514 | hsa-miR-527 | 1024 | 5 | 4.32e−014 |
| 4042 | 39 | 148 | 283 | hsa-miR-557 | 1034 | 5 | 2.57e−013 |
| 4043 | 39 | 56 | 338 | hsa-miR-564 | 859 | 5 | 1.54e−013 |
| 4044 | 39 | 135 | 315 | hsa-miR-565 | 6145 | 5 | 9.57e−014 |
| 4045 | 39 | 55 | 319 | hsa-miR-593 | 598 | 5 | 1.1e−013 |
| 4046 | 39 | 12 | 275 | hsa-miR-634 | 698 | 5 | 7.01e−013 |
| 4047 | 39 | 60 | 479 | hsa-miR-638 | 30572 | 5 | 2.23e−013 |
| 4048 | 39 | 133 | 412 | hsa-miR-658 | 738 | 5 | 2.98e−013 |
| 4049 | 39 | 195 | 421 | hsa-miR-662 | 695 | 5 | 1.2e−014 |
| 4050 | 39 | 230 | 474, 484, 536 | hsa-miR-7 | 974 | 5 | 4.32e−014 |
| 4051 | 39 | 182 | 304, 317 | hsa-miR-92 | 2932 | 5 | 6.21e−014 |
| 4052 | 39 | 6 | 471 | hsa-miR-93 | 1672 | 5 | 2.15e−016 |
| 4053 | 39 | 221 | 307 | hsa-miR-98 | 599 | 5 | 3.6e−015 |
| 4054 | 39 | 14 | 318 | hsa-miR-99a | 18639 | 5 | 1.26e−014 |
| 4055 | 39 | 80 | 509 | hsa-miR-99b | 2415 | 5 | 3.02e−013 |
| 4056 | 39 | 22 | 453 | c14 + 290a | 680 | 5 | 2.53e−016 |
| 4057 | 39 | 131 | 430 | 6_11 | 1091 | 5 | 3.8e−014 |
| 4058 | 39 | 23 | 546 | 47_6 | 621 | 5 | 3.94e−014 |
| 4059 | 40 | 127 | 361 | 11_1 | 859 | 6 | 1.59e−016 |
| 4060 | 40 | 196 | 508 | 15_rel | 58698 | 6 | 2.32e−016 |
| 4061 | 40 | 197 | 444 | 25_6 | 544 | 6 | 3.51e−017 |
| 4062 | 40 | 219 | 497 | 27_3 | 2673 | 6 | 2.55e−016 |
| 4063 | 40 | 122 | 281 | 43_11 | 615 | 6 | 2.12e−017 |
| 4064 | 40 | 7 | 337 | 43_21 | 625 | 6 | 1.46e−017 |
| 4065 | 40 | 42 | 449 | 43_3 | 793 | 6 | 7.43e−017 |
| 4066 | 40 | 101 | 475 | 43_30 | 641 | 6 | 1.2e−017 |
| 4067 | 40 | 99 | 348, 384, 434 | 43_35 | 641 | 6 | 3.13e−015 |
| 4068 | 40 | 129 | 548 | 43_39 | 1998 | 6 | 3.84e−016 |
| 4069 | 40 | 211 | 353 | 43_44 | 514 | 6 | 1.17e−015 |
| 4070 | 40 | 114 | 530 | 43_6 | 814 | 6 | 5.56e−017 |
| 4071 | 40 | 183 | 373 | 66_1 | 935 | 6 | 3.1e−017 |
| 4072 | 40 | 94 | 394 | 69_6_rel | 2834 | 6 | 1.83e−011 |
| 4073 | 40 | 121 | 502 | 70_14 | 979 | 6 | 1.07e−015 |
| 4074 | 40 | 177 | 366, 464 | 72_12 | 1889 | 6 | 3.65e−014 |
| 4075 | 40 | 40 | 553 | 72_4 | 998 | 6 | 4.09e−013 |
| 4076 | 40 | 97 | 357 | 74_26_rel | 4330 | 6 | 1.38e−016 |
| 4077 | 40 | 87 | 301 | gam11 | 786 | 6 | 1.5e−011 |
| 4078 | 40 | 163 | 402 | 43_7 | 1314 | 6 | 2.52e−017 |
| 4079 | 40 | 223 | 320, 393, 559 | hsa-let-7a | 4171 | 6 | 2.26e−014 |
| 4080 | 40 | 225 | 332 | hsa-let-7b | 22397 | 6 | 1.53e−017 |
| 4081 | 40 | 224 | 427 | hsa-let-7c | 12893 | 6 | 1.05e−014 |
| 4082 | 40 | 45 | 277 | hsa-let-7d | 2285 | 6 | 8.67e−018 |
| 4083 | 40 | 220 | 475 | hsa-let-7e | 3125 | 6 | 1.39e−019 |
| 4084 | 40 | 222 | 482, 558 | hsa-let-7f | 662 | 6 | 6.25e−017 |
| 4085 | 40 | 226 | 395 | hsa-let-7g | 1826 | 6 | 2.48e−016 |
| 4086 | 40 | 227 | 297 | hsa-let-7i | 2058 | 6 | 9.81e−016 |
| 4087 | 40 | 231 | 321, 424 | hsa-miR-1 | 791 | 6 | 5.73e−017 |
| 4088 | 40 | 13 | 397 | hsa-miR-100 | 843 | 6 | 3.04e−012 |
| 4089 | 40 | 184 | 423 | hsa-miR-105 | 1180 | 6 | 2.54e−017 |
| 4090 | 40 | 2 | 399 | hsa-miR-106a | 1593 | 6 | 8.15e−016 |
| 4091 | 40 | 149 | 392 | hsa-miR-106b | 1273 | 6 | 1.95e−017 |
| 4092 | 40 | 47 | 555 | hsa-miR-107 | 1750 | 6 | 1.17e−016 |
| 4093 | 40 | 235 | 358 | hsa-miR-122a | 547 | 6 | 1.47e−016 |
| 4094 | 40 | 199 | 387 | hsa-miR-125a | 3511 | 6 | 1.58e−015 |
| 4095 | 40 | 198 | 313, 551 | hsa-miR-125b | 20503 | 6 | 1.4e−015 |
| 4096 | 40 | 204 | 419 | hsa-miR-126 | 896 | 6 | 1.63e−015 |
| 4097 | 40 | 89 | 419 | hsa-miR-126* | 726 | 6 | 2.13e−018 |
| 4098 | 40 | 86 | 417 | hsa-miR-130a | 2756 | 6 | 2.99e−017 |
| 4099 | 40 | 85 | 328 | hsa-miR-130b | 701 | 6 | 3.17e−017 |
| 4100 | 40 | 153 | 445 | hsa-miR-132 | 646 | 6 | 1.33e−017 |
| 4101 | 40 | 266 | 295, 386 | hsa-miR-133a | 1304 | 6 | 4.22e−016 |
| 4102 | 40 | 265 | 533 | hsa-miR-133b | 1285 | 6 | 6.02e−017 |
| 4103 | 40 | 180 | 486 | hsa-miR-135b | 704 | 6 | 2.04e−016 |
| 4104 | 40 | 52 | 370, 548 | hsa-miR-138 | 521 | 6 | 1.27e−013 |
| 4105 | 40 | 64 | 402 | hsa-miR-140 | 823 | 6 | 5.43e−015 |
| 4106 | 40 | 151 | 472 | hsa-miR-141 | 5428 | 6 | 4.68e−015 |
| 4107 | 40 | 218 | 527 | hsa-miR-143 | 2599 | 6 | 1.72e−011 |
| 4108 | 40 | 139 | 281 | hsa-miR-145 | 44637 | 6 | 2.73e−018 |
| 4109 | 40 | 146 | 325 | hsa-miR-147 | 705 | 6 | 4.69e−016 |
| 4110 | 40 | 190 | 458 | hsa-miR-148a | 522 | 6 | 2.17e−016 |
| 4111 | 40 | 191 | 289 | hsa-miR-148b | 625 | 6 | 6.72e−017 |
| 4112 | 40 | 212 | 333 | hsa-miR-149 | 1430 | 6 | 3.32e−018 |
| 4113 | 40 | 209 | 507 | hsa-miR-150 | 665 | 6 | 5.32e−017 |
| 4114 | 40 | 34 | 396 | hsa-miR-151 | 914 | 6 | 6.39e−017 |
| 4115 | 40 | 192 | 470 | hsa-miR-152 | 843 | 6 | 1.43e−017 |
| 4116 | 40 | 263 | 296, 404 | hsa-miR-153 | 606 | 6 | 1.47e−015 |
| 4117 | 40 | 253 | 499 | hsa-miR-155 | 641 | 6 | 1.62e−016 |
| 4118 | 40 | 171 | 331 | hsa-miR-15a | 607 | 6 | 1.03e−017 |
| 4119 | 40 | 173 | 360, 468 | hsa-miR-16 | 1608 | 6 | 1.85e−014 |
| 4120 | 40 | 38 | 467 | hsa-miR-17-3p | 636 | 6 | 2.63e−019 |
| 4121 | 40 | 75 | 467 | hsa-miR-17-5p | 1173 | 6 | 1.46e−015 |
| 4122 | 40 | 10 | 371, 461 | hsa-miR-181a | 1327 | 6 | 1.51e−016 |
| 4123 | 40 | 11 | 456, 540 | hsa-miR-181b | 502 | 6 | 6.47e−018 |
| 4124 | 40 | 179 | 356 | hsa-miR-183 | 532 | 6 | 1.22e−012 |
| 4125 | 40 | 233 | 305 | hsa-miR-184 | 601 | 6 | 8.31e−017 |
| 4126 | 40 | 234 | 450 | hsa-miR-185 | 902 | 6 | 4.44e−017 |
| 4127 | 40 | 205 | 367 | hsa-miR-187 | 546 | 6 | 2.25e−017 |
| 4128 | 40 | 88 | 310 | hsa-miR-188 | 872 | 6 | 1.98e−016 |
| 4129 | 40 | 144 | 346 | hsa-miR-189 | 815 | 6 | 4.2e−018 |
| 4130 | 40 | 77 | 440 | hsa-miR-191 | 2357 | 6 | 1.39e−016 |
| 4131 | 40 | 130 | 440 | hsa-miR-191* | 845 | 6 | 3.1e−015 |
| 4132 | 40 | 17 | 442 | hsa-miR-193a | 2766 | 6 | 1.64e−015 |
| 4133 | 40 | 136 | 308 | hsa-miR-198 | 802 | 6 | 3.34e−018 |
| 4134 | 40 | 92 | 410, 432 | hsa-miR-199a | 1509 | 6 | 9.72e−013 |
| 4135 | 40 | 162 | 410, 432, 473 | hsa-miR-199a* | 594 | 6 | 9.11e−015 |
| 4136 | 40 | 156 | 446 | hsa-miR-200c | 6578 | 6 | 1.82e−016 |
| 4137 | 40 | 271 | 334 | hsa-miR-202* | 1025 | 6 | 4.37e−017 |
| 4138 | 40 | 203 | 504 | hsa-miR-205 | 11087 | 6 | 1.1e−016 |
| 4139 | 40 | 232 | 422 | hsa-miR-206 | 1170 | 6 | 2.97e−015 |
| 4140 | 40 | 175 | 462 | hsa-miR-21 | 3786 | 6 | 7.08e−015 |
| 4141 | 40 | 115 | 314 | hsa-miR-210 | 711 | 6 | 1.72e−016 |
| 4142 | 40 | 154 | 300 | hsa-miR-212 | 602 | 6 | 8.88e−016 |
| 4143 | 40 | 29 | 439 | hsa-miR-214 | 3072 | 6 | 2.02e−016 |
| 4144 | 40 | 159 | 494 | hsa-miR-216 | 954 | 6 | 1.57e−016 |
| 4145 | 40 | 268 | 401, 425 | hsa-miR-218 | 843 | 6 | 5.92e−016 |
| 4146 | 40 | 18 | 322 | hsa-miR-22 | 3322 | 6 | 5.03e−015 |
| 4147 | 40 | 50 | 364 | hsa-miR-221 | 5278 | 6 | 8.11e−015 |
| 4148 | 40 | 49 | 302 | hsa-miR-222 | 7889 | 6 | 9.29e−016 |
| 4149 | 40 | 248 | 415 | hsa-miR-223 | 641 | 6 | 4.32e−017 |
| 4150 | 40 | 67 | 336 | hsa-miR-23a | 1693 | 6 | 2.32e−014 |
| 4151 | 40 | 238 | 346, 416 | hsa-miR-24 | 11022 | 6 | 7.48e−016 |
| 4152 | 40 | 256 | 441, 487 | hsa-miR-26a | 5723 | 6 | 1.05e−014 |
| 4153 | 40 | 259 | 481 | hsa-miR-27a | 1933 | 6 | 7.3e−015 |
| 4154 | 40 | 260 | 280 | hsa-miR-27b | 1944 | 6 | 9.73e−015 |
| 4155 | 40 | 269 | 362 | hsa-miR-302c* | 718 | 6 | 1.95e−017 |
| 4156 | 40 | 117 | 287 | hsa-miR-30a-3p | 837 | 6 | 5.96e−018 |
| 4157 | 40 | 242 | 311 | hsa-miR-30b | 660 | 6 | 1.94e−014 |
| 4158 | 40 | 243 | 299, 316 | hsa-miR-30c | 1456 | 6 | 1.35e−014 |
| 4159 | 40 | 241 | 550 | hsa-miR-30d | 2013 | 6 | 1.53e−017 |
| 4160 | 40 | 116 | 431 | hsa-miR-30e-3p | 625 | 6 | 3.68e−016 |
| 4161 | 40 | 245 | 431 | hsa-miR-30e-5p | 2421 | 6 | 2.02e−016 |
| 4162 | 40 | 132 | 429 | hsa-miR-31 | 1309 | 6 | 3.06e−016 |
| 4163 | 40 | 1 | 359 | hsa-miR-320 | 5468 | 6 | 2.68e−016 |
| 4164 | 40 | 124 | 455 | hsa-miR-323 | 664 | 6 | 3.48e−016 |
| 4165 | 40 | 91 | 368 | hsa-miR-324-3p | 1005 | 6 | 1.3e−016 |
| 4166 | 40 | 98 | 368 | hsa-miR-324-5p | 1155 | 6 | 9.41e−016 |
| 4167 | 40 | 113 | 538 | hsa-miR-328 | 1406 | 6 | 3.95e−016 |
| 4168 | 40 | 141 | 437 | hsa-miR-33 | 1756 | 6 | 1.56e−016 |
| 4169 | 40 | 126 | 380 | hsa-miR-331 | 723 | 6 | 3.38e−018 |
| 4170 | 40 | 185 | 492 | hsa-miR-335 | 655 | 6 | 9.2e−017 |
| 4171 | 40 | 208 | 477 | hsa-miR-342 | 1521 | 6 | 6.89e−018 |
| 4172 | 40 | 229 | 292 | hsa-miR-345 | 1268 | 6 | 2.42e−017 |
| 4173 | 40 | 237 | 480 | hsa-miR-34a | 1973 | 6 | 1.19e−016 |
| 4174 | 40 | 160 | 379, 382 | hsa-miR-365 | 972 | 6 | 2.35e−017 |
| 4175 | 40 | 31 | 560 | hsa-miR-368 | 510 | 6 | 1.87e−017 |
| 4176 | 40 | 128 | 294 | hsa-miR-370 | 1438 | 6 | 1.2e−017 |
| 4177 | 40 | 254 | 496 | hsa-miR-374 | 776 | 6 | 1.08e−016 |
| 4178 | 40 | 105 | 372 | hsa-miR-378 | 648 | 6 | 1.06e−016 |
| 4179 | 40 | 178 | 352 | hsa-miR-381 | 585 | 6 | 1.86e−017 |
| 4180 | 40 | 46 | 298 | hsa-miR-383 | 739 | 6 | 3.28e−016 |
| 4181 | 40 | 111 | 389 | hsa-miR-422a | 711 | 6 | 2.31e−016 |
| 4182 | 40 | 112 | 372 | hsa-miR-422b | 1718 | 6 | 3.21e−016 |
| 4183 | 40 | 51 | 497 | hsa-miR-423 | 3235 | 6 | 2.18e−017 |
| 4184 | 40 | 82 | 377 | hsa-miR-424 | 515 | 6 | 3.18e−017 |
| 4185 | 40 | 69 | 363 | hsa-miR-425-3p | 615 | 6 | 7.6e−016 |
| 4186 | 40 | 213 | 531 | hsa-miR-432 | 687 | 6 | 1.82e−015 |
| 4187 | 40 | 236 | 413 | hsa-miR-449 | 4173 | 6 | 1.84e−015 |
| 4188 | 40 | 274 | 375, 309 | hsa-miR-450 | 664 | 6 | 1.4e−015 |
| 4189 | 40 | 252 | 405 | hsa-miR-452 | 1351 | 6 | 4.96e−015 |
| 4190 | 40 | 138 | 547 | hsa-miR-485-3p | 545 | 6 | 2.38e−015 |
| 4191 | 40 | 62 | 485 | hsa-miR-489 | 595 | 6 | 6.87e−017 |
| 4192 | 40 | 63 | 557 | hsa-miR-491 | 4814 | 6 | 2.55e−016 |
| 4193 | 40 | 54 | 293 | hsa-miR-492 | 1204 | 6 | 3.24e−016 |
| 4194 | 40 | 267 | 354 | hsa-miR-493-5p | 720 | 6 | 2.27e−015 |
| 4195 | 40 | 214 | 376 | hsa-miR-494 | 13046 | 6 | 2.9e−016 |
| 4196 | 40 | 3 | 543 | hsa-miR-495 | 748 | 6 | 1.57e−015 |
| 4197 | 40 | 83 | 312 | hsa-miR-497 | 1969 | 6 | 6.2e−016 |
| 4198 | 40 | 25 | 414 | hsa-miR-501 | 685 | 6 | 3.75e−016 |
| 4199 | 40 | 68 | 288 | hsa-miR-502 | 636 | 6 | 1.05e−016 |
| 4200 | 40 | 43 | 418 | hsa-miR-504 | 1288 | 6 | 2.75e−016 |
| 4201 | 40 | 258 | 428, 443 | hsa-miR-513 | 2126 | 6 | 1.1e−016 |
| 4202 | 40 | 73 | 503 | hsa-miR-518b | 797 | 6 | 1.22e−015 |
| 4203 | 40 | 210 | 400 | hsa-miR-518c* | 1256 | 6 | 1.28e−016 |
| 4204 | 40 | 262 | 523 | hsa-miR-519e* | 864 | 6 | 9.99e−017 |
| 4205 | 40 | 102 | 344 | hsa-miR-520a* | 618 | 6 | 8.82e−018 |
| 4206 | 40 | 206 | 513 | hsa-miR-520d* | 704 | 6 | 2.55e−013 |
| 4207 | 40 | 100 | 524 | hsa-miR-524* | 939 | 6 | 2.42e−016 |
| 4208 | 40 | 118 | 342 | hsa-miR-525* | 725 | 6 | 7.53e−015 |
| 4209 | 40 | 110 | 511, 512, 514 | hsa-miR-527 | 763 | 6 | 9.66e−017 |
| 4210 | 40 | 148 | 283 | hsa-miR-557 | 821 | 6 | 4.56e−017 |
| 4211 | 40 | 56 | 338 | hsa-miR-564 | 611 | 6 | 4.84e−017 |
| 4212 | 40 | 135 | 315 | hsa-miR-565 | 6221 | 6 | 8.51e−016 |
| 4213 | 40 | 60 | 479 | hsa-miR-638 | 20163 | 6 | 2.84e−016 |
| 4214 | 40 | 195 | 421 | hsa-miR-662 | 513 | 6 | 2.37e−016 |
| 4215 | 40 | 230 | 474, 484, 536 | hsa-miR-7 | 760 | 6 | 2.45e−015 |
| 4216 | 40 | 182 | 304, 317 | hsa-miR-92 | 2682 | 6 | 1.97e−016 |
| 4217 | 40 | 6 | 471 | hsa-miR-93 | 1454 | 6 | 7.05e−017 |
| 4218 | 40 | 14 | 318 | hsa-miR-99a | 13508 | 6 | 1.57e−015 |
| 4219 | 40 | 80 | 509 | hsa-miR-99b | 1829 | 6 | 8.91e−017 |
| 4220 | 40 | 22 | 453 | c14 + 290a | 584 | 6 | 5.07e−018 |
| 4221 | 40 | 131 | 430 | 6_11 | 694 | 6 | 1.05e−015 |
| 4222 | 41 | 127 | 361 | 11_1 | 980 | 7 | 5.46e−018 |
| 4223 | 41 | 196 | 508 | 15_rel | 31930 | 7 | 2.48e−019 |
| 4224 | 41 | 219 | 497 | 27_3 | 1085 | 7 | 4.13e−018 |
| 4225 | 41 | 145 | 447 | 43_44_rel | 47302 | 7 | 1.38e−019 |
| 4226 | 41 | 94 | 394 | 69_6_rel | 2683 | 7 | 3.86e−016 |
| 4227 | 41 | 177 | 366, 464 | 72_12 | 883 | 7 | 2.33e−017 |
| 4228 | 41 | 40 | 553 | 72_4 | 649 | 7 | 1.73e−016 |
| 4229 | 41 | 97 | 357 | 74_26_rel | 622 | 7 | 2.61e−018 |
| 4230 | 41 | 33 | 339, 552 | 96_rel | 22494 | 7 | 9.41e−019 |
| 4231 | 41 | 87 | 301 | gam11 | 6089 | 7 | 6.17e−017 |
| 4232 | 41 | 223 | 320, 393, 559 | hsa-let-7a | 1813 | 7 | 1.03e−015 |
| 4233 | 41 | 223 | 320, 393, 559 | hsa-let-7a | 560 | 7 | 8.52e−017 |
| 4234 | 41 | 225 | 332 | hsa-let-7b | 10329 | 7 | 1.6e−016 |
| 4235 | 41 | 224 | 427 | hsa-let-7c | 2473 | 7 | 3.27e−016 |
| 4236 | 41 | 45 | 277 | hsa-let-7d | 1668 | 7 | 1.54e−017 |
| 4237 | 41 | 220 | 475 | hsa-let-7e | 2260 | 7 | 1.61e−018 |
| 4238 | 41 | 226 | 395 | hsa-let-7g | 591 | 7 | 1.18e−020 |
| 4239 | 41 | 227 | 297 | hsa-let-7i | 1269 | 7 | 2.18e−017 |
| 4240 | 41 | 13 | 397 | hsa-miR-100 | 952 | 7 | 1.2e−018 |
| 4241 | 41 | 48 | 483, 529 | hsa-miR-103 | 4799 | 7 | 1.35e−017 |
| 4242 | 41 | 184 | 423 | hsa-miR-105 | 983 | 7 | 3.8e−018 |
| 4243 | 41 | 2 | 399 | hsa-miR-106a | 711 | 7 | 1.17e−018 |
| 4244 | 41 | 149 | 392 | hsa-miR-106b | 804 | 7 | 2.99e−019 |
| 4245 | 41 | 47 | 555 | hsa-miR-107 | 3852 | 7 | 1.35e−017 |
| 4246 | 41 | 199 | 387 | hsa-miR-125a | 3303 | 7 | 6.37e−018 |
| 4247 | 41 | 198 | 313, 551 | hsa-miR-125b | 3645 | 7 | 2.48e−018 |
| 4248 | 41 | 204 | 419 | hsa-miR-126 | 950 | 7 | 1.32e−018 |
| 4249 | 41 | 86 | 417 | hsa-miR-130a | 633 | 7 | 5.24e−019 |
| 4250 | 41 | 151 | 472 | hsa-miR-141 | 2545 | 7 | 4.01e−017 |
| 4251 | 41 | 218 | 527 | hsa-miR-143 | 9434 | 7 | 2.41e−016 |
| 4252 | 41 | 139 | 281 | hsa-miR-145 | 14031 | 7 | 3.72e−016 |
| 4253 | 41 | 173 | 360, 468 | hsa-miR-16 | 1651 | 7 | 3.15e−016 |
| 4254 | 41 | 10 | 371, 461 | hsa-miR-181a | 880 | 7 | 2.58e−019 |
| 4255 | 41 | 272 | 323 | hsa-miR-182 | 824 | 7 | 1.39e−014 |
| 4256 | 41 | 179 | 356 | hsa-miR-183 | 589 | 7 | 3.3e−016 |
| 4257 | 41 | 234 | 450 | hsa-miR-185 | 634 | 7 | 4.86e−019 |
| 4258 | 41 | 77 | 440 | hsa-miR-191 | 1248 | 7 | 1.51e−018 |
| 4259 | 41 | 130 | 440 | hsa-miR-191* | 683 | 7 | 8.35e−017 |
| 4260 | 41 | 109 | 374 | hsa-miR-192 | 1567 | 7 | 2.91e−016 |
| 4261 | 41 | 17 | 442 | hsa-miR-193a | 1415 | 7 | 6.21e−020 |
| 4262 | 41 | 246 | 290, 549 | hsa-miR-194 | 4846 | 7 | 1.54e−017 |
| 4263 | 41 | 92 | 410, 432 | hsa-miR-199a | 2058 | 7 | 5.9e−021 |
| 4264 | 41 | 152 | 369 | hsa-miR-200a | 2108 | 7 | 8.59e−016 |
| 4265 | 41 | 157 | 411 | hsa-miR-200b | 1932 | 7 | 7.14e−016 |
| 4266 | 41 | 156 | 446 | hsa-miR-200c | 4874 | 7 | 1.15e−017 |
| 4267 | 41 | 175 | 462 | hsa-miR-21 | 1727 | 7 | 1.64e−015 |
| 4268 | 41 | 115 | 314 | hsa-miR-210 | 516 | 7 | 1.7e−018 |
| 4269 | 41 | 29 | 439 | hsa-miR-214 | 1184 | 7 | 4.39e−018 |
| 4270 | 41 | 18 | 322 | hsa-miR-22 | 2056 | 7 | 2.11e−017 |
| 4271 | 41 | 50 | 364 | hsa-miR-221 | 931 | 7 | 9.67e−017 |
| 4272 | 41 | 49 | 302 | hsa-miR-222 | 1253 | 7 | 9.6e−017 |
| 4273 | 41 | 67 | 336 | hsa-miR-23a | 1904 | 7 | 5.11e−017 |
| 4274 | 41 | 238 | 346, 416 | hsa-miR-24 | 8915 | 7 | 5.72e−020 |
| 4275 | 41 | 256 | 441, 487 | hsa-miR-26a | 5460 | 7 | 8.4e−017 |
| 4276 | 41 | 259 | 481 | hsa-miR-27a | 1919 | 7 | 3.76e−018 |
| 4277 | 41 | 260 | 280 | hsa-miR-27b | 1200 | 7 | 2.24e−017 |
| 4278 | 41 | 61 | 286 | hsa-miR-296 | 1161 | 7 | 6.71e−017 |
| 4279 | 41 | 167 | 556 | hsa-miR-29a | 1061 | 7 | 1.97e−016 |
| 4280 | 41 | 244 | 287 | hsa-miR-30a-5p | 719 | 7 | 1.05e−017 |
| 4281 | 41 | 241 | 550 | hsa-miR-30d | 1590 | 7 | 3.71e−016 |
| 4282 | 41 | 245 | 431 | hsa-miR-30e-5p | 856 | 7 | 1.14e−016 |
| 4283 | 41 | 132 | 429 | hsa-miR-31 | 921 | 7 | 1.74e−016 |
| 4284 | 41 | 1 | 359 | hsa-miR-320 | 4684 | 7 | 5.33e−019 |
| 4285 | 41 | 91 | 368 | hsa-miR-324-3p | 1051 | 7 | 1.76e−019 |
| 4286 | 41 | 98 | 368 | hsa-miR-324-5p | 940 | 7 | 4.07e−018 |
| 4287 | 41 | 113 | 538 | hsa-miR-328 | 527 | 7 | 7.88e−018 |
| 4288 | 41 | 141 | 437 | hsa-miR-33 | 881 | 7 | 4.99e−017 |
| 4289 | 41 | 126 | 380 | hsa-miR-331 | 940 | 7 | 4.81e−019 |
| 4290 | 41 | 194 | 337 | hsa-miR-338 | 1678 | 7 | 4.55e−017 |
| 4291 | 41 | 208 | 477 | hsa-miR-342 | 874 | 7 | 1.68e−018 |
| 4292 | 41 | 237 | 480 | hsa-miR-34a | 1045 | 7 | 1.8e−018 |
| 4293 | 41 | 255 | 444 | hsa-miR-361 | 566 | 7 | 2.9e−020 |
| 4294 | 41 | 273 | 335 | hsa-miR-375 | 7408 | 7 | 7.61e−016 |
| 4295 | 41 | 112 | 372 | hsa-miR-422b | 671 | 7 | 9.56e−017 |
| 4296 | 41 | 51 | 497 | hsa-miR-423 | 1470 | 7 | 2.24e−019 |
| 4297 | 41 | 236 | 413 | hsa-miR-449 | 574 | 7 | 1.03e−017 |
| 4298 | 41 | 62 | 485 | hsa-miR-489 | 501 | 7 | 2.57e−018 |
| 4299 | 41 | 63 | 557 | hsa-miR-491 | 1235 | 7 | 1.14e−016 |
| 4300 | 41 | 214 | 376 | hsa-miR-494 | 2026 | 7 | 7.91e−016 |
| 4301 | 41 | 83 | 312 | hsa-miR-497 | 946 | 7 | 9.53e−020 |
| 4302 | 41 | 135 | 315 | hsa-miR-565 | 745 | 7 | 6.34e−017 |
| 4303 | 41 | 60 | 479 | hsa-miR-638 | 3018 | 7 | 4.54e−016 |
| 4304 | 41 | 59 | 330 | hsa-miR-663 | 714 | 7 | 7.67e−017 |
| 4305 | 41 | 230 | 474, 484, 536 | hsa-miR-7 | 535 | 7 | 4.68e−018 |
| 4306 | 41 | 186 | 341 | hsa-miR-768-3p | 1963 | 7 | 5.44e−018 |
| 4307 | 41 | 147 | 341 | hsa-miR-768-5p | 626 | 7 | 1.33e−018 |
| 4308 | 41 | 123 | 291 | hsa-miR-801 | 1091 | 7 | 2.72e−018 |
| 4309 | 41 | 182 | 304, 317 | hsa-miR-92 | 1110 | 7 | 5.77e−018 |
| 4310 | 41 | 6 | 471 | hsa-miR-93 | 844 | 7 | 1.4e−019 |
| 4311 | 41 | 14 | 318 | hsa-miR-99a | 864 | 7 | 6.19e−019 |
| 4312 | 41 | 80 | 509 | hsa-miR-99b | 1399 | 7 | 5.85e−019 |
| 4313 | 42 | 127 | 361 | 11_1 | 533 | 11 | 2.03e−024 |
| 4314 | 42 | 196 | 508 | 15_rel | 36601 | 11 | 1.95e−024 |
| 4315 | 42 | 219 | 497 | 27_3 | 1022 | 11 | 3.89e−028 |
| 4316 | 42 | 145 | 447 | 43_44_rel | 58160 | 7 | 6.02e−017 |
| 4317 | 42 | 94 | 394 | 69_6_rel | 4510 | 11 | 1.26e−023 |
| 4318 | 42 | 177 | 366, 464 | 72_12 | 1386 | 11 | 1.35e−023 |
| 4319 | 42 | 40 | 553 | 72_4 | 614 | 11 | 3.16e−025 |
| 4320 | 42 | 97 | 357 | 74_26_rel | 724 | 11 | 2.77e−022 |
| 4321 | 42 | 33 | 339, 552 | 96_rel | 40199 | 7 | 6.47e−018 |
| 4322 | 42 | 87 | 301 | gam11 | 10092 | 11 | 2.37e−025 |
| 4323 | 42 | 223 | 320, 393, 559 | hsa-let-7a | 2141 | 11 | 1.82e−024 |
| 4324 | 42 | 223 | 320, 393, 559 | hsa-let-7a | 686 | 11 | 5.98e−024 |
| 4325 | 42 | 225 | 332 | hsa-let-7b | 5525 | 11 | 2.19e−026 |
| 4326 | 42 | 224 | 427 | hsa-let-7c | 3050 | 11 | 3.06e−025 |
| 4327 | 42 | 45 | 277 | hsa-let-7d | 1102 | 11 | 4.49e−028 |
| 4328 | 42 | 220 | 475 | hsa-let-7e | 1299 | 11 | 5.48e−027 |
| 4329 | 42 | 226 | 395 | hsa-let-7g | 602 | 11 | 1.86e−027 |
| 4330 | 42 | 227 | 297 | hsa-let-7i | 780 | 11 | 3.02e−026 |
| 4331 | 42 | 13 | 397 | hsa-miR-100 | 729 | 11 | 2.3e−026 |
| 4332 | 42 | 48 | 483, 529 | hsa-miR-103 | 1549 | 11 | 9.7e−025 |
| 4333 | 42 | 184 | 423 | hsa-miR-105 | 598 | 11 | 2.55e−029 |
| 4334 | 42 | 2 | 399 | hsa-miR-106a | 1212 | 11 | 1.53e−027 |
| 4335 | 42 | 149 | 392 | hsa-miR-106b | 793 | 11 | 5e−029 |
| 4336 | 42 | 47 | 555 | hsa-miR-107 | 1335 | 11 | 9.91e−026 |
| 4337 | 42 | 199 | 387 | hsa-miR-125a | 1433 | 11 | 7.6e−026 |
| 4338 | 42 | 198 | 313, 551 | hsa-miR-125b | 2903 | 11 | 2.63e−025 |
| 4339 | 42 | 204 | 419 | hsa-miR-126 | 1215 | 11 | 1.11e−025 |
| 4340 | 42 | 86 | 417 | hsa-miR-130a | 974 | 11 | 9.81e−030 |
| 4341 | 42 | 266 | 295, 386 | hsa-miR-133a | 556 | 11 | 1.02e−026 |
| 4342 | 42 | 151 | 472 | hsa-miR-141 | 2935 | 11 | 3.23e−026 |
| 4343 | 42 | 218 | 527 | hsa-miR-143 | 11827 | 11 | 8.14e−025 |
| 4344 | 42 | 139 | 281 | hsa-miR-145 | 12652 | 11 | 4.26e−025 |
| 4345 | 42 | 209 | 507 | hsa-miR-150 | 793 | 11 | 1.41e−026 |
| 4346 | 42 | 173 | 360, 468 | hsa-miR-16 | 1840 | 11 | 4.63e−026 |
| 4347 | 42 | 75 | 467 | hsa-miR-17-5p | 987 | 11 | 1.79e−027 |
| 4348 | 42 | 10 | 371, 461 | hsa-miR-181a | 1326 | 11 | 1.55e−029 |
| 4349 | 42 | 77 | 440 | hsa-miR-191 | 1120 | 11 | 2.8e−024 |
| 4350 | 42 | 130 | 440 | hsa-miR-191* | 565 | 11 | 9.09e−027 |
| 4351 | 42 | 109 | 374 | hsa-miR-192 | 2996 | 11 | 9.05e−024 |
| 4352 | 42 | 17 | 442 | hsa-miR-193a | 1072 | 11 | 2.9e−025 |
| 4353 | 42 | 246 | 290, 549 | hsa-miR-194 | 9787 | 11 | 7.33e−025 |
| 4354 | 42 | 92 | 410, 432 | hsa-miR-199a | 1664 | 11 | 1.33e−025 |
| 4355 | 42 | 251 | 282, 495 | hsa-miR-19b | 760 | 11 | 1.22e−025 |
| 4356 | 42 | 152 | 369 | hsa-miR-200a | 1112 | 11 | 2.22e−026 |
| 4357 | 42 | 157 | 411 | hsa-miR-200b | 1607 | 11 | 1.29e−025 |
| 4358 | 42 | 156 | 446 | hsa-miR-200c | 4098 | 11 | 2.47e−025 |
| 4359 | 42 | 175 | 462 | hsa-miR-21 | 2882 | 11 | 3.91e−024 |
| 4360 | 42 | 29 | 439 | hsa-miR-214 | 908 | 11 | 1.11e−026 |
| 4361 | 42 | 18 | 322 | hsa-miR-22 | 2807 | 11 | 6.79e−027 |
| 4362 | 42 | 50 | 364 | hsa-miR-221 | 1554 | 11 | 5.19e−028 |
| 4363 | 42 | 49 | 302 | hsa-miR-222 | 1684 | 11 | 4.88e−027 |
| 4364 | 42 | 67 | 336 | hsa-miR-23a | 1784 | 11 | 3.61e−026 |
| 4365 | 42 | 66 | 306 | hsa-miR-23b | 808 | 11 | 3.03e−019 |
| 4366 | 42 | 238 | 346, 416 | hsa-miR-24 | 5768 | 11 | 1.68e−025 |
| 4367 | 42 | 256 | 441, 487 | hsa-miR-26a | 3852 | 11 | 4.02e−026 |
| 4368 | 42 | 259 | 481 | hsa-miR-27a | 2010 | 11 | 2.73e−025 |
| 4369 | 42 | 260 | 280 | hsa-miR-27b | 910 | 11 | 1.72e−025 |
| 4370 | 42 | 61 | 286 | hsa-miR-296 | 1360 | 11 | 3.96e−026 |
| 4371 | 42 | 167 | 556 | hsa-miR-29a | 975 | 11 | 3.57e−025 |
| 4372 | 42 | 244 | 287 | hsa-miR-30a-5p | 904 | 11 | 1.83e−027 |
| 4373 | 42 | 243 | 299, 316 | hsa-miR-30c | 646 | 11 | 1.98e−022 |
| 4374 | 42 | 241 | 550 | hsa-miR-30d | 1088 | 11 | 3.35e−028 |
| 4375 | 42 | 245 | 431 | hsa-miR-30e-5p | 932 | 11 | 1.75e−022 |
| 4376 | 42 | 132 | 429 | hsa-miR-31 | 3456 | 11 | 6.24e−027 |
| 4377 | 42 | 1 | 359 | hsa-miR-320 | 3458 | 11 | 1.04e−027 |
| 4378 | 42 | 113 | 538 | hsa-miR-328 | 667 | 11 | 6.31e−028 |
| 4379 | 42 | 141 | 437 | hsa-miR-33 | 615 | 11 | 4.58e−024 |
| 4380 | 42 | 126 | 380 | hsa-miR-331 | 755 | 11 | 9.53e−029 |
| 4381 | 42 | 185 | 492 | hsa-miR-335 | 1264 | 11 | 9.49e−025 |
| 4382 | 42 | 194 | 337 | hsa-miR-338 | 1597 | 11 | 2.2e−024 |
| 4383 | 42 | 208 | 477 | hsa-miR-342 | 591 | 11 | 5.25e−025 |
| 4384 | 42 | 237 | 480 | hsa-miR-34a | 817 | 11 | 1.14e−029 |
| 4385 | 42 | 128 | 294 | hsa-miR-370 | 506 | 11 | 6.86e−027 |
| 4386 | 42 | 112 | 372 | hsa-miR-422b | 1543 | 11 | 1.47e−025 |
| 4387 | 42 | 51 | 497 | hsa-miR-423 | 1514 | 11 | 2.17e−029 |
| 4388 | 42 | 236 | 413 | hsa-miR-449 | 1084 | 11 | 2.26e−023 |
| 4389 | 42 | 63 | 557 | hsa-miR-491 | 1396 | 11 | 4.56e−024 |
| 4390 | 42 | 214 | 376 | hsa-miR-494 | 7430 | 11 | 8.5e−022 |
| 4391 | 42 | 83 | 312 | hsa-miR-497 | 855 | 11 | 4.71e−027 |
| 4392 | 42 | 135 | 315 | hsa-miR-565 | 1258 | 11 | 5.48e−026 |
| 4393 | 42 | 60 | 479 | hsa-miR-638 | 4379 | 11 | 8.45e−024 |
| 4394 | 42 | 59 | 330 | hsa-miR-663 | 949 | 11 | 1.65e−022 |
| 4395 | 42 | 186 | 341 | hsa-miR-768-3p | 638 | 10 | 1.49e−019 |
| 4396 | 42 | 123 | 291 | hsa-miR-801 | 1228 | 10 | 3.76e−024 |
| 4397 | 42 | 182 | 304, 317 | hsa-miR-92 | 2284 | 11 | 4.65e−029 |
| 4398 | 42 | 6 | 471 | hsa-miR-93 | 1141 | 11 | 6e−029 |
| 4399 | 42 | 14 | 318 | hsa-miR-99a | 639 | 11 | 2.57e−025 |
| 4400 | 42 | 80 | 509 | hsa-miR-99b | 622 | 11 | 9.53e−027 |
| 4401 | 43 | 127 | 361 | 11_1 | 1253 | 6 | 2.93e−012 |
| 4402 | 43 | 196 | 508 | 15_rel | 63202 | 6 | 1.88e−013 |
| 4403 | 43 | 219 | 497 | 27_3 | 1132 | 6 | 5.84e−014 |
| 4404 | 43 | 27 | 313 | 43_40 | 525 | 6 | 1.33e−012 |
| 4405 | 43 | 145 | 447 | 43_44_rel | 63552 | 6 | 2.03e−013 |
| 4406 | 43 | 94 | 394 | 69_6_rel | 5004 | 6 | 5.54e−013 |
| 4407 | 43 | 177 | 366, 464 | 72_12 | 5228 | 6 | 5e−013 |
| 4408 | 43 | 40 | 553 | 72_4 | 1613 | 6 | 8.24e−013 |
| 4409 | 43 | 97 | 357 | 74_26_rel | 1398 | 6 | 1.45e−012 |
| 4410 | 43 | 33 | 339, 552 | 96_rel | 51621 | 6 | 8.18e−013 |
| 4411 | 43 | 87 | 301 | gam11 | 27053 | 6 | 6.92e−012 |
| 4412 | 43 | 125 | 532 | 70_16 | 1813 | 6 | 1.08e−013 |
| 4413 | 43 | 163 | 402 | 43_7 | 939 | 6 | 1.07e−011 |
| 4414 | 43 | 223 | 320, 393, 559 | hsa-let-7a | 12501 | 6 | 2.5e−011 |
| 4415 | 43 | 223 | 320, 393, 559 | hsa-let-7a | 4314 | 6 | 9.69e−011 |
| 4416 | 43 | 225 | 332 | hsa-let-7b | 33212 | 6 | 2.02e−012 |
| 4417 | 43 | 224 | 427 | hsa-let-7c | 22007 | 6 | 1.16e−011 |
| 4418 | 43 | 45 | 277 | hsa-let-7d | 5025 | 6 | 2.18e−013 |
| 4419 | 43 | 220 | 475 | hsa-let-7e | 7683 | 6 | 3.34e−013 |
| 4420 | 43 | 222 | 482, 558 | hsa-let-7f | 784 | 6 | 2.41e−012 |
| 4421 | 43 | 226 | 395 | hsa-let-7g | 1024 | 6 | 1.01e−012 |
| 4422 | 43 | 227 | 297 | hsa-let-7i | 1973 | 6 | 1.41e−012 |
| 4423 | 43 | 13 | 397 | hsa-miR-100 | 5248 | 6 | 1.89e−011 |
| 4424 | 43 | 48 | 483, 529 | hsa-miR-103 | 4132 | 6 | 3.21e−011 |
| 4425 | 43 | 184 | 423 | hsa-miR-105 | 1325 | 6 | 5.78e−014 |
| 4426 | 43 | 2 | 399 | hsa-miR-106a | 1386 | 6 | 6.52e−013 |
| 4427 | 43 | 149 | 392 | hsa-miR-106b | 1245 | 6 | 3.6e−013 |
| 4428 | 43 | 47 | 555 | hsa-miR-107 | 3494 | 6 | 3.64e−011 |
| 4429 | 43 | 199 | 387 | hsa-miR-125a | 10446 | 6 | 4.95e−014 |
| 4430 | 43 | 198 | 313, 551 | hsa-miR-125b | 35268 | 6 | 7.13e−015 |
| 4431 | 43 | 204 | 419 | hsa-miR-126 | 1420 | 6 | 3.57e−013 |
| 4432 | 43 | 86 | 417 | hsa-miR-130a | 1396 | 6 | 9.73e−013 |
| 4433 | 43 | 153 | 445 | hsa-miR-132 | 848 | 6 | 1.68e−013 |
| 4434 | 43 | 207 | 355 | hsa-miR-139 | 768 | 6 | 1.55e−009 |
| 4435 | 43 | 218 | 527 | hsa-miR-143 | 63222 | 6 | 1.88e−013 |
| 4436 | 43 | 139 | 281 | hsa-miR-145 | 62050 | 6 | 3.24e−013 |
| 4437 | 43 | 191 | 289 | hsa-miR-148b | 561 | 6 | 8.43e−013 |
| 4438 | 43 | 212 | 333 | hsa-miR-149 | 1673 | 6 | 5.59e−012 |
| 4439 | 43 | 209 | 507 | hsa-miR-150 | 509 | 6 | 8.65e−013 |
| 4440 | 43 | 34 | 396 | hsa-miR-151 | 667 | 6 | 6.53e−013 |
| 4441 | 43 | 192 | 470 | hsa-miR-152 | 661 | 6 | 1.29e−011 |
| 4442 | 43 | 172 | 329 | hsa-miR-15b | 1069 | 6 | 8.45e−013 |
| 4443 | 43 | 173 | 360, 468 | hsa-miR-16 | 4338 | 6 | 1.46e−012 |
| 4444 | 43 | 75 | 467 | hsa-miR-17-5p | 1218 | 6 | 1.81e−013 |
| 4445 | 43 | 10 | 371, 461 | hsa-miR-181a | 6423 | 6 | 1.99e−012 |
| 4446 | 43 | 11 | 456, 540 | hsa-miR-181b | 1170 | 6 | 1.19e−013 |
| 4447 | 43 | 234 | 450 | hsa-miR-185 | 694 | 6 | 2.53e−013 |
| 4448 | 43 | 205 | 367 | hsa-miR-187 | 764 | 6 | 2.76e−010 |
| 4449 | 43 | 77 | 440 | hsa-miR-191 | 3299 | 6 | 4.7e−012 |
| 4450 | 43 | 130 | 440 | hsa-miR-191* | 754 | 6 | 1.06e−012 |
| 4451 | 43 | 17 | 442 | hsa-miR-193a | 1059 | 6 | 3.03e−012 |
| 4452 | 43 | 16 | 488 | hsa-miR-193b | 2331 | 6 | 1.86e−011 |
| 4453 | 43 | 170 | 327 | hsa-miR-195 | 825 | 6 | 3.38e−011 |
| 4454 | 43 | 92 | 410, 432 | hsa-miR-199a | 11304 | 6 | 4.58e−013 |
| 4455 | 43 | 251 | 282, 495 | hsa-miR-19b | 885 | 6 | 4.98e−013 |
| 4456 | 43 | 175 | 462 | hsa-miR-21 | 2169 | 6 | 1.55e−011 |
| 4457 | 43 | 115 | 314 | hsa-miR-210 | 702 | 6 | 1.27e−012 |
| 4458 | 43 | 29 | 439 | hsa-miR-214 | 4074 | 6 | 2.86e−012 |
| 4459 | 43 | 18 | 322 | hsa-miR-22 | 6512 | 6 | 2.32e−013 |
| 4460 | 43 | 50 | 364 | hsa-miR-221 | 1211 | 6 | 1.94e−011 |
| 4461 | 43 | 49 | 302 | hsa-miR-222 | 1282 | 6 | 1.29e−011 |
| 4462 | 43 | 67 | 336 | hsa-miR-23a | 5091 | 6 | 2.99e−013 |
| 4463 | 43 | 66 | 306 | hsa-miR-23b | 3491 | 6 | 2.43e−009 |
| 4464 | 43 | 238 | 346, 416 | hsa-miR-24 | 17050 | 6 | 2.9e−013 |
| 4465 | 43 | 90 | 388 | hsa-miR-25 | 584 | 6 | 1.5e−013 |
| 4466 | 43 | 256 | 441, 487 | hsa-miR-26a | 18005 | 6 | 3.64e−013 |
| 4467 | 43 | 259 | 481 | hsa-miR-27a | 5434 | 6 | 1.2e−012 |
| 4468 | 43 | 260 | 280 | hsa-miR-27b | 1149 | 6 | 1.02e−012 |
| 4469 | 43 | 61 | 286 | hsa-miR-296 | 1192 | 6 | 1.16e−013 |
| 4470 | 43 | 167 | 556 | hsa-miR-29a | 2996 | 6 | 8.99e−012 |
| 4471 | 43 | 168 | 303, 530 | hsa-miR-29b | 876 | 6 | 1.83e−011 |
| 4472 | 43 | 169 | 528 | hsa-miR-29c | 516 | 6 | 4.2e−012 |
| 4473 | 43 | 117 | 287 | hsa-miR-30a-3p | 533 | 6 | 3.78e−014 |
| 4474 | 43 | 244 | 287 | hsa-miR-30a-5p | 7280 | 6 | 9.16e−012 |
| 4475 | 43 | 242 | 311 | hsa-miR-30b | 1050 | 6 | 2.45e−011 |
| 4476 | 43 | 243 | 299, 316 | hsa-miR-30c | 3482 | 6 | 9.29e−012 |
| 4477 | 43 | 241 | 550 | hsa-miR-30d | 2890 | 6 | 3.1e−012 |
| 4478 | 43 | 245 | 431 | hsa-miR-30e-5p | 878 | 6 | 6.09e−010 |
| 4479 | 43 | 132 | 429 | hsa-miR-31 | 617 | 6 | 3.42e−011 |
| 4480 | 43 | 1 | 359 | hsa-miR-320 | 8951 | 6 | 2.21e−011 |
| 4481 | 43 | 91 | 368 | hsa-miR-324-3p | 1477 | 6 | 1.72e−012 |
| 4482 | 43 | 98 | 368 | hsa-miR-324-5p | 761 | 6 | 3.53e−011 |
| 4483 | 43 | 113 | 538 | hsa-miR-328 | 1237 | 6 | 2.73e−013 |
| 4484 | 43 | 141 | 437 | hsa-miR-33 | 1702 | 6 | 4.08e−013 |
| 4485 | 43 | 126 | 380 | hsa-miR-331 | 1436 | 6 | 1.7e−013 |
| 4486 | 43 | 194 | 337 | hsa-miR-338 | 3849 | 6 | 9.67e−012 |
| 4487 | 43 | 200 | 448 | hsa-miR-339 | 644 | 6 | 8.44e−012 |
| 4488 | 43 | 208 | 477 | hsa-miR-342 | 759 | 6 | 3.88e−013 |
| 4489 | 43 | 237 | 480 | hsa-miR-34a | 2472 | 6 | 1.03e−012 |
| 4490 | 43 | 255 | 444 | hsa-miR-361 | 700 | 6 | 1.7e−013 |
| 4491 | 43 | 128 | 294 | hsa-miR-370 | 507 | 6 | 1.28e−011 |
| 4492 | 43 | 46 | 298 | hsa-miR-383 | 534 | 6 | 6.12e−011 |
| 4493 | 43 | 112 | 372 | hsa-miR-422b | 846 | 6 | 1.49e−012 |
| 4494 | 43 | 51 | 497 | hsa-miR-423 | 2391 | 6 | 8.42e−014 |
| 4495 | 43 | 236 | 413 | hsa-miR-449 | 1231 | 6 | 3.22e−011 |
| 4496 | 43 | 63 | 557 | hsa-miR-491 | 1233 | 6 | 3.33e−013 |
| 4497 | 43 | 214 | 376 | hsa-miR-494 | 11259 | 6 | 1.08e−010 |
| 4498 | 43 | 83 | 312 | hsa-miR-497 | 2121 | 6 | 2.36e−011 |
| 4499 | 43 | 21 | 517, 519 | hsa-miR-520f | 606 | 6 | 5.74e−013 |
| 4500 | 43 | 135 | 315 | hsa-miR-565 | 1370 | 6 | 7.34e−011 |
| 4501 | 43 | 60 | 479 | hsa-miR-638 | 4261 | 6 | 7.42e−014 |
| 4502 | 43 | 59 | 330 | hsa-miR-663 | 1494 | 6 | 5.42e−013 |
| 4503 | 43 | 36 | 385 | hsa-miR-766 | 953 | 6 | 6.98e−012 |
| 4504 | 43 | 186 | 341 | hsa-miR-768-3p | 3316 | 6 | 2.97e−013 |
| 4505 | 43 | 147 | 341 | hsa-miR-768-5p | 1106 | 6 | 1.06e−011 |
| 4506 | 43 | 123 | 291 | hsa-miR-801 | 2081 | 6 | 6.85e−013 |
| 4507 | 43 | 182 | 304, 317 | hsa-miR-92 | 1983 | 6 | 6.52e−012 |
| 4508 | 43 | 6 | 471 | hsa-miR-93 | 2030 | 6 | 2.53e−012 |
| 4509 | 43 | 14 | 318 | hsa-miR-99a | 5888 | 6 | 7.51e−014 |
| 4510 | 43 | 80 | 509 | hsa-miR-99b | 2763 | 6 | 8.79e−012 |
| 4511 | 43 | 131 | 430 | 6_11 | 718 | 6 | 3.41e−012 |
| 4512 | 44 | 127 | 361 | 11_1 | 851 | 17 | 0 |
| 4513 | 44 | 196 | 508 | 15_rel | 39760 | 17 | 0 |
| 4514 | 44 | 219 | 497 | 27_3 | 1053 | 17 | 0 |
| 4515 | 44 | 145 | 447 | 43_44_rel | 45092 | 14 | 4.01e−033 |
| 4516 | 44 | 94 | 394 | 69_6_rel | 3170 | 17 | 0 |
| 4517 | 44 | 177 | 366, 464 | 72_12 | 1456 | 17 | 6.61e−037 |
| 4518 | 44 | 40 | 553 | 72_4 | 818 | 17 | 0 |
| 4519 | 44 | 97 | 357 | 74_26_rel | 943 | 17 | 0 |
| 4520 | 44 | 33 | 339, 552 | 96_rel | 26917 | 14 | 6.56e−036 |
| 4521 | 44 | 87 | 301 | gam11 | 10090 | 17 | 0 |
| 4522 | 44 | 223 | 320, 393, 559 | hsa-let-7a | 2927 | 17 | 1.09e−035 |
| 4523 | 44 | 223 | 320, 393, 559 | hsa-let-7a | 971 | 17 | 2.7e−035 |
| 4524 | 44 | 225 | 332 | hsa-let-7b | 11831 | 17 | 0 |
| 4525 | 44 | 224 | 427 | hsa-let-7c | 4961 | 17 | 2.85e−036 |
| 4526 | 44 | 45 | 277 | hsa-let-7d | 1964 | 17 | 0 |
| 4527 | 44 | 220 | 475 | hsa-let-7e | 2781 | 17 | 0 |
| 4528 | 44 | 226 | 395 | hsa-let-7g | 612 | 17 | 0 |
| 4529 | 44 | 227 | 297 | hsa-let-7i | 1170 | 17 | 0 |
| 4530 | 44 | 13 | 397 | hsa-miR-100 | 1452 | 17 | 7.61e−038 |
| 4531 | 44 | 48 | 483, 529 | hsa-miR-103 | 3500 | 17 | 1.86e−037 |
| 4532 | 44 | 184 | 423 | hsa-miR-105 | 888 | 17 | 0 |
| 4533 | 44 | 2 | 399 | hsa-miR-106a | 1020 | 17 | 0 |
| 4534 | 44 | 149 | 392 | hsa-miR-106b | 967 | 17 | 0 |
| 4535 | 44 | 47 | 555 | hsa-miR-107 | 2907 | 17 | 3.94e−038 |
| 4536 | 44 | 199 | 387 | hsa-miR-125a | 3195 | 17 | 1.74e−037 |
| 4537 | 44 | 198 | 313, 551 | hsa-miR-125b | 7320 | 17 | 2.83e−038 |
| 4538 | 44 | 204 | 419 | hsa-miR-126 | 982 | 17 | 0 |
| 4539 | 44 | 86 | 417 | hsa-miR-130a | 902 | 17 | 0 |
| 4540 | 44 | 151 | 472 | hsa-miR-141 | 1195 | 17 | 6.85e−032 |
| 4541 | 44 | 218 | 527 | hsa-miR-143 | 12511 | 17 | 5.4e−035 |
| 4542 | 44 | 139 | 281 | hsa-miR-145 | 15510 | 17 | 3.12e−035 |
| 4543 | 44 | 212 | 333 | hsa-miR-149 | 510 | 17 | 3.9e−038 |
| 4544 | 44 | 173 | 360, 468 | hsa-miR-16 | 2136 | 17 | 0 |
| 4545 | 44 | 75 | 467 | hsa-miR-17-5p | 763 | 17 | 0 |
| 4546 | 44 | 10 | 371, 461 | hsa-miR-181a | 1742 | 17 | 2.07e−038 |
| 4547 | 44 | 11 | 456, 540 | hsa-miR-181b | 520 | 17 | 0 |
| 4548 | 44 | 234 | 450 | hsa-miR-185 | 612 | 17 | 0 |
| 4549 | 44 | 77 | 440 | hsa-miR-191 | 1952 | 17 | 0 |
| 4550 | 44 | 130 | 440 | hsa-miR-191* | 565 | 17 | 0 |
| 4551 | 44 | 109 | 374 | hsa-miR-192 | 686 | 17 | 1.3e−033 |
| 4552 | 44 | 17 | 442 | hsa-miR-193a | 1342 | 17 | 1.36e−038 |
| 4553 | 44 | 16 | 488 | hsa-miR-193b | 756 | 17 | 2.78e−037 |
| 4554 | 44 | 246 | 290, 549 | hsa-miR-194 | 1547 | 17 | 9.87e−032 |
| 4555 | 44 | 92 | 410, 432 | hsa-miR-199a | 2892 | 17 | 7.23e−038 |
| 4556 | 44 | 251 | 282, 495 | hsa-miR-19b | 687 | 17 | 1.78e−038 |
| 4557 | 44 | 152 | 369 | hsa-miR-200a | 964 | 17 | 3.32e−033 |
| 4558 | 44 | 157 | 411 | hsa-miR-200b | 912 | 17 | 3.4e−032 |
| 4559 | 44 | 156 | 446 | hsa-miR-200c | 1827 | 17 | 3.93e−031 |
| 4560 | 44 | 175 | 462 | hsa-miR-21 | 2390 | 17 | 4.01e−035 |
| 4561 | 44 | 115 | 314 | hsa-miR-210 | 551 | 17 | 0 |
| 4562 | 44 | 29 | 439 | hsa-miR-214 | 1492 | 17 | 0 |
| 4563 | 44 | 18 | 322 | hsa-miR-22 | 2734 | 17 | 0 |
| 4564 | 44 | 50 | 364 | hsa-miR-221 | 1297 | 17 | 2.42e−036 |
| 4565 | 44 | 49 | 302 | hsa-miR-222 | 1528 | 17 | 1.23e−036 |
| 4566 | 44 | 67 | 336 | hsa-miR-23a | 2659 | 17 | 0 |
| 4567 | 44 | 66 | 306 | hsa-miR-23b | 1323 | 17 | 4.81e−030 |
| 4568 | 44 | 238 | 346, 416 | hsa-miR-24 | 10881 | 17 | 0 |
| 4569 | 44 | 256 | 441, 487 | hsa-miR-26a | 6810 | 17 | 0 |
| 4570 | 44 | 259 | 481 | hsa-miR-27a | 2846 | 17 | 0 |
| 4571 | 44 | 260 | 280 | hsa-miR-27b | 987 | 17 | 0 |
| 4572 | 44 | 61 | 286 | hsa-miR-296 | 1111 | 17 | 0 |
| 4573 | 44 | 167 | 556 | hsa-miR-29a | 1493 | 17 | 2.05e−038 |
| 4574 | 44 | 244 | 287 | hsa-miR-30a-5p | 1489 | 17 | 1.41e−036 |
| 4575 | 44 | 243 | 299, 316 | hsa-miR-30c | 710 | 17 | 4.32e−033 |
| 4576 | 44 | 241 | 550 | hsa-miR-30d | 1662 | 17 | 0 |
| 4577 | 44 | 245 | 431 | hsa-miR-30e-5p | 911 | 17 | 1.11e−035 |
| 4578 | 44 | 132 | 429 | hsa-miR-31 | 752 | 17 | 1.74e−036 |
| 4579 | 44 | 1 | 359 | hsa-miR-320 | 5781 | 17 | 0 |
| 4580 | 44 | 91 | 368 | hsa-miR-324-3p | 956 | 17 | 0 |
| 4581 | 44 | 98 | 368 | hsa-miR-324-5p | 688 | 17 | 0 |
| 4582 | 44 | 113 | 538 | hsa-miR-328 | 621 | 17 | 0 |
| 4583 | 44 | 141 | 437 | hsa-miR-33 | 840 | 17 | 3.88e−037 |
| 4584 | 44 | 126 | 380 | hsa-miR-331 | 994 | 17 | 0 |
| 4585 | 44 | 194 | 337 | hsa-miR-338 | 2430 | 17 | 0 |
| 4586 | 44 | 208 | 477 | hsa-miR-342 | 741 | 17 | 0 |
| 4587 | 44 | 237 | 480 | hsa-miR-34a | 1345 | 17 | 0 |
| 4588 | 44 | 255 | 444 | hsa-miR-361 | 563 | 17 | 0 |
| 4589 | 44 | 112 | 372 | hsa-miR-422b | 772 | 17 | 0 |
| 4590 | 44 | 51 | 497 | hsa-miR-423 | 1602 | 17 | 0 |
| 4591 | 44 | 236 | 413 | hsa-miR-449 | 757 | 17 | 1.32e−038 |
| 4592 | 44 | 63 | 557 | hsa-miR-491 | 1220 | 17 | 0 |
| 4593 | 44 | 214 | 376 | hsa-miR-494 | 3873 | 17 | 1.55e−035 |
| 4594 | 44 | 83 | 312 | hsa-miR-497 | 936 | 17 | 0 |
| 4595 | 44 | 135 | 315 | hsa-miR-565 | 1079 | 17 | 6.57e−037 |
| 4596 | 44 | 60 | 479 | hsa-miR-638 | 3422 | 17 | 0 |
| 4597 | 44 | 59 | 330 | hsa-miR-663 | 1075 | 17 | 0 |
| 4598 | 44 | 186 | 341 | hsa-miR-768-3p | 1397 | 17 | 4.86e−034 |
| 4599 | 44 | 147 | 341 | hsa-miR-768-5p | 668 | 17 | 0 |
| 4600 | 44 | 123 | 291 | hsa-miR-801 | 1234 | 17 | 0 |
| 4601 | 44 | 182 | 304, 317 | hsa-miR-92 | 1776 | 17 | 5.03e−036 |
| 4602 | 44 | 6 | 471 | hsa-miR-93 | 1411 | 17 | 2.17e−038 |
| 4603 | 44 | 14 | 318 | hsa-miR-99a | 1641 | 17 | 2.01e−037 |
| 4604 | 44 | 80 | 509 | hsa-miR-99b | 1340 | 17 | 0 |
| 4605 | 45 | 127 | 361 | 11_1 | 862 | 6 | 7.06e−014 |
| 4606 | 45 | 196 | 508 | 15_rel | 54935 | 6 | 3.9e−014 |
| 4607 | 45 | 219 | 497 | 27_3 | 1131 | 6 | 1.12e−013 |
| 4608 | 45 | 145 | 447 | 43_44_rel | 50960 | 5 | 4.9e−012 |
| 4609 | 45 | 94 | 394 | 69_6_rel | 4190 | 6 | 1.55e−013 |
| 4610 | 45 | 177 | 366, 464 | 72_12 | 3230 | 6 | 1.48e−012 |
| 4611 | 45 | 40 | 553 | 72_4 | 1187 | 6 | 9.22e−015 |
| 4612 | 45 | 97 | 357 | 74_26_rel | 1497 | 6 | 7.4e−013 |
| 4613 | 45 | 33 | 339, 552 | 96_rel | 41134 | 5 | 1.01e−012 |
| 4614 | 45 | 87 | 301 | gam11 | 18168 | 6 | 2.78e−013 |
| 4615 | 45 | 125 | 532 | 70_16 | 603 | 6 | 8.48e−012 |
| 4616 | 45 | 163 | 402 | 43_7 | 502 | 6 | 1.35e−011 |
| 4617 | 45 | 223 | 320, 393, 559 | hsa-let-7a | 3886 | 6 | 3.51e−012 |
| 4618 | 45 | 223 | 320, 393, 559 | hsa-let-7a | 1206 | 6 | 1.54e−011 |
| 4619 | 45 | 225 | 332 | hsa-let-7b | 19381 | 6 | 3.54e−013 |
| 4620 | 45 | 224 | 427 | hsa-let-7c | 8794 | 6 | 5.53e−013 |
| 4621 | 45 | 45 | 277 | hsa-let-7d | 2454 | 6 | 3.88e−013 |
| 4622 | 45 | 220 | 475 | hsa-let-7e | 3749 | 6 | 1e−012 |
| 4623 | 45 | 226 | 395 | hsa-let-7g | 607 | 6 | 6.78e−014 |
| 4624 | 45 | 227 | 297 | hsa-let-7i | 1125 | 6 | 6.24e−014 |
| 4625 | 45 | 13 | 397 | hsa-miR-100 | 2941 | 6 | 9.75e−013 |
| 4626 | 45 | 48 | 483, 529 | hsa-miR-103 | 3329 | 6 | 1.05e−012 |
| 4627 | 45 | 184 | 423 | hsa-miR-105 | 808 | 6 | 3.16e−014 |
| 4628 | 45 | 2 | 399 | hsa-miR-106a | 1549 | 6 | 2.42e−013 |
| 4629 | 45 | 149 | 392 | hsa-miR-106b | 1060 | 6 | 4.39e−014 |
| 4630 | 45 | 47 | 555 | hsa-miR-107 | 3029 | 6 | 3.73e−013 |
| 4631 | 45 | 199 | 387 | hsa-miR-125a | 4332 | 6 | 1.49e−012 |
| 4632 | 45 | 198 | 313, 551 | hsa-miR-125b | 16568 | 6 | 3.7e−013 |
| 4633 | 45 | 204 | 419 | hsa-miR-126 | 1001 | 6 | 4.99e−013 |
| 4634 | 45 | 86 | 417 | hsa-miR-130a | 1107 | 6 | 1.97e−013 |
| 4635 | 45 | 218 | 527 | hsa-miR-143 | 26283 | 6 | 4.41e−012 |
| 4636 | 45 | 139 | 281 | hsa-miR-145 | 30247 | 6 | 1.25e−012 |
| 4637 | 45 | 212 | 333 | hsa-miR-149 | 1043 | 6 | 7.48e−013 |
| 4638 | 45 | 34 | 396 | hsa-miR-151 | 605 | 6 | 8.48e−015 |
| 4639 | 45 | 173 | 360, 468 | hsa-miR-16 | 2809 | 6 | 1.31e−013 |
| 4640 | 45 | 75 | 467 | hsa-miR-17-5p | 1281 | 6 | 8.95e−014 |
| 4641 | 45 | 10 | 371, 461 | hsa-miR-181a | 4449 | 6 | 6.19e−014 |
| 4642 | 45 | 11 | 456, 540 | hsa-miR-181b | 795 | 6 | 4.25e−014 |
| 4643 | 45 | 234 | 450 | hsa-miR-185 | 610 | 6 | 1.94e−013 |
| 4644 | 45 | 77 | 440 | hsa-miR-191 | 2946 | 6 | 1.07e−013 |
| 4645 | 45 | 130 | 440 | hsa-miR-191* | 532 | 6 | 1.12e−013 |
| 4646 | 45 | 17 | 442 | hsa-miR-193a | 1441 | 6 | 1.74e−012 |
| 4647 | 45 | 16 | 488 | hsa-miR-193b | 1634 | 6 | 1.39e−012 |
| 4648 | 45 | 246 | 290, 549 | hsa-miR-194 | 702 | 6 | 1.11e−010 |
| 4649 | 45 | 92 | 410, 432 | hsa-miR-199a | 5695 | 6 | 1.02e−012 |
| 4650 | 45 | 251 | 282, 495 | hsa-miR-19b | 1075 | 6 | 3.53e−012 |
| 4651 | 45 | 156 | 446 | hsa-miR-200c | 669 | 6 | 2.76e−010 |
| 4652 | 45 | 175 | 462 | hsa-miR-21 | 2454 | 6 | 1.86e−011 |
| 4653 | 45 | 115 | 314 | hsa-miR-210 | 587 | 6 | 6.52e−013 |
| 4654 | 45 | 29 | 439 | hsa-miR-214 | 2533 | 6 | 3.26e−013 |
| 4655 | 45 | 18 | 322 | hsa-miR-22 | 5074 | 6 | 1.87e−014 |
| 4656 | 45 | 50 | 364 | hsa-miR-221 | 1498 | 6 | 2e−011 |
| 4657 | 45 | 49 | 302 | hsa-miR-222 | 1600 | 6 | 1.2e−011 |
| 4658 | 45 | 67 | 336 | hsa-miR-23a | 3254 | 6 | 2.3e−014 |
| 4659 | 45 | 66 | 306 | hsa-miR-23b | 2399 | 6 | 1.18e−010 |
| 4660 | 45 | 238 | 346, 416 | hsa-miR-24 | 12138 | 6 | 1.72e−013 |
| 4661 | 45 | 256 | 441, 487 | hsa-miR-26a | 8144 | 6 | 1.54e−013 |
| 4662 | 45 | 259 | 481 | hsa-miR-27a | 3488 | 6 | 5.09e−014 |
| 4663 | 45 | 260 | 280 | hsa-miR-27b | 693 | 6 | 2.38e−014 |
| 4664 | 45 | 61 | 286 | hsa-miR-296 | 1010 | 6 | 3.27e−014 |
| 4665 | 45 | 167 | 556 | hsa-miR-29a | 2040 | 6 | 1.17e−012 |
| 4666 | 45 | 244 | 287 | hsa-miR-30a-5p | 3440 | 6 | 3.07e−012 |
| 4667 | 45 | 243 | 299, 316 | hsa-miR-30c | 1281 | 6 | 4.45e−012 |
| 4668 | 45 | 241 | 550 | hsa-miR-30d | 1767 | 6 | 9.65e−015 |
| 4669 | 45 | 245 | 431 | hsa-miR-30e-5p | 1118 | 6 | 9.66e−013 |
| 4670 | 45 | 132 | 429 | hsa-miR-31 | 573 | 6 | 1.54e−011 |
| 4671 | 45 | 1 | 359 | hsa-miR-320 | 10077 | 6 | 1.57e−013 |
| 4672 | 45 | 91 | 368 | hsa-miR-324-3p | 1138 | 6 | 2.28e−014 |
| 4673 | 45 | 98 | 368 | hsa-miR-324-5p | 667 | 6 | 9.92e−014 |
| 4674 | 45 | 113 | 538 | hsa-miR-328 | 779 | 6 | 5.97e−014 |
| 4675 | 45 | 141 | 437 | hsa-miR-33 | 654 | 6 | 1.44e−011 |
| 4676 | 45 | 126 | 380 | hsa-miR-331 | 1004 | 6 | 1.76e−014 |
| 4677 | 45 | 194 | 337 | hsa-miR-338 | 4128 | 6 | 1.94e−012 |
| 4678 | 45 | 208 | 477 | hsa-miR-342 | 618 | 6 | 3.15e−013 |
| 4679 | 45 | 237 | 480 | hsa-miR-34a | 2184 | 6 | 4.53e−014 |
| 4680 | 45 | 255 | 444 | hsa-miR-361 | 546 | 6 | 1.5e−014 |
| 4681 | 45 | 128 | 294 | hsa-miR-370 | 551 | 6 | 4.01e−014 |
| 4682 | 45 | 112 | 372 | hsa-miR-422b | 838 | 6 | 7.86e−014 |
| 4683 | 45 | 51 | 497 | hsa-miR-423 | 2130 | 6 | 1.4e−014 |
| 4684 | 45 | 236 | 413 | hsa-miR-449 | 1169 | 6 | 3.91e−012 |
| 4685 | 45 | 63 | 557 | hsa-miR-491 | 1366 | 6 | 4.21e−013 |
| 4686 | 45 | 214 | 376 | hsa-miR-494 | 6599 | 6 | 8.33e−012 |
| 4687 | 45 | 83 | 312 | hsa-miR-497 | 969 | 6 | 3.5e−012 |
| 4688 | 45 | 135 | 315 | hsa-miR-565 | 2108 | 6 | 4.92e−012 |
| 4689 | 45 | 60 | 479 | hsa-miR-638 | 4374 | 6 | 6.25e−013 |
| 4690 | 45 | 195 | 421 | hsa-miR-662 | 535 | 6 | 6.43e−011 |
| 4691 | 45 | 59 | 330 | hsa-miR-663 | 1511 | 6 | 4.71e−013 |
| 4692 | 45 | 186 | 341 | hsa-miR-768-3p | 1246 | 6 | 4.45e−011 |
| 4693 | 45 | 147 | 341 | hsa-miR-768-5p | 800 | 6 | 2.01e−013 |
| 4694 | 45 | 123 | 291 | hsa-miR-801 | 1386 | 6 | 5.17e−014 |
| 4695 | 45 | 182 | 304, 317 | hsa-miR-92 | 3764 | 6 | 1.33e−011 |
| 4696 | 45 | 6 | 471 | hsa-miR-93 | 2238 | 6 | 4.15e−013 |
| 4697 | 45 | 14 | 318 | hsa-miR-99a | 2443 | 6 | 3.28e−013 |
| 4698 | 45 | 80 | 509 | hsa-miR-99b | 1628 | 6 | 8.75e−013 |
| 4699 | 46 | 127 | 361 | 11_1 | 612 | 5 | 1.79e−011 |
| 4700 | 46 | 196 | 508 | 15_rel | 49233 | 5 | 3.74e−014 |
| 4701 | 46 | 219 | 497 | 27_3 | 1176 | 5 | 3.61e−012 |
| 4702 | 46 | 145 | 447 | 43_44_rel | 27655 | 5 | 9.4e−010 |
| 4703 | 46 | 94 | 394 | 69_6_rel | 7745 | 5 | 3.93e−012 |
| 4704 | 46 | 177 | 366, 464 | 72_12 | 2195 | 5 | 2.66e−010 |
| 4705 | 46 | 97 | 357 | 74_26_rel | 2499 | 5 | 8.91e−012 |
| 4706 | 46 | 33 | 339, 552 | 96_rel | 38265 | 5 | 1.22e−012 |
| 4707 | 46 | 87 | 301 | gam11 | 20967 | 5 | 1.16e−011 |
| 4708 | 46 | 223 | 320, 393, 559 | hsa-let-7a | 3164 | 5 | 3.02e−010 |
| 4709 | 46 | 223 | 320, 393, 559 | hsa-let-7a | 912 | 5 | 1.17e−009 |
| 4710 | 46 | 225 | 332 | hsa-let-7b | 8539 | 5 | 4e−012 |
| 4711 | 46 | 224 | 427 | hsa-let-7c | 6574 | 5 | 3.65e−010 |
| 4712 | 46 | 45 | 277 | hsa-let-7d | 1203 | 5 | 2.86e−012 |
| 4713 | 46 | 220 | 475 | hsa-let-7e | 1439 | 5 | 1.38e−012 |
| 4714 | 46 | 226 | 395 | hsa-let-7g | 1000 | 5 | 4.13e−012 |
| 4715 | 46 | 227 | 297 | hsa-let-7i | 1583 | 5 | 2.72e−011 |
| 4716 | 46 | 13 | 397 | hsa-miR-100 | 932 | 5 | 5.6e−012 |
| 4717 | 46 | 48 | 483, 529 | hsa-miR-103 | 1768 | 5 | 1.91e−012 |
| 4718 | 46 | 184 | 423 | hsa-miR-105 | 642 | 5 | 3.4e−012 |
| 4719 | 46 | 2 | 399 | hsa-miR-106a | 1542 | 5 | 4e−012 |
| 4720 | 46 | 149 | 392 | hsa-miR-106b | 1254 | 5 | 1.57e−012 |
| 4721 | 46 | 47 | 555 | hsa-miR-107 | 1502 | 5 | 2.95e−012 |
| 4722 | 46 | 199 | 387 | hsa-miR-125a | 1816 | 5 | 3e−012 |
| 4723 | 46 | 198 | 313, 551 | hsa-miR-125b | 6831 | 5 | 5.39e−012 |
| 4724 | 46 | 204 | 419 | hsa-miR-126 | 905 | 5 | 6.18e−012 |
| 4725 | 46 | 89 | 419 | hsa-miR-126* | 606 | 5 | 2.93e−011 |
| 4726 | 46 | 86 | 417 | hsa-miR-130a | 1896 | 5 | 1.38e−011 |
| 4727 | 46 | 64 | 402 | hsa-miR-140 | 596 | 5 | 1.69e−012 |
| 4728 | 46 | 151 | 472 | hsa-miR-141 | 5324 | 5 | 1.73e−011 |
| 4729 | 46 | 218 | 527 | hsa-miR-143 | 8322 | 5 | 2.77e−011 |
| 4730 | 46 | 139 | 281 | hsa-miR-145 | 8749 | 5 | 8.49e−011 |
| 4731 | 46 | 212 | 333 | hsa-miR-149 | 717 | 5 | 3.58e−012 |
| 4732 | 46 | 209 | 507 | hsa-miR-150 | 927 | 5 | 3.46e−011 |
| 4733 | 46 | 34 | 396 | hsa-miR-151 | 537 | 5 | 2.23e−011 |
| 4734 | 46 | 172 | 329 | hsa-miR-15b | 700 | 5 | 1.43e−011 |
| 4735 | 46 | 173 | 360, 468 | hsa-miR-16 | 2772 | 5 | 2.56e−010 |
| 4736 | 46 | 38 | 467 | hsa-miR-17-3p | 510 | 5 | 1.07e−012 |
| 4737 | 46 | 75 | 467 | hsa-miR-17-5p | 1198 | 5 | 1.18e−011 |
| 4738 | 46 | 10 | 371, 461 | hsa-miR-181a | 1871 | 5 | 5.1e−013 |
| 4739 | 46 | 179 | 356 | hsa-miR-183 | 1103 | 5 | 3.53e−011 |
| 4740 | 46 | 234 | 450 | hsa-miR-185 | 544 | 5 | 7.32e−013 |
| 4741 | 46 | 77 | 440 | hsa-miR-191 | 2007 | 5 | 3.31e−011 |
| 4742 | 46 | 130 | 440 | hsa-miR-191* | 568 | 5 | 5.78e−011 |
| 4743 | 46 | 109 | 374 | hsa-miR-192 | 1237 | 5 | 2.67e−010 |
| 4744 | 46 | 17 | 442 | hsa-miR-193a | 1023 | 5 | 3.3e−012 |
| 4745 | 46 | 16 | 488 | hsa-miR-193b | 1010 | 5 | 3.09e−012 |
| 4746 | 46 | 246 | 290, 549 | hsa-miR-194 | 3070 | 5 | 5.29e−010 |
| 4747 | 46 | 92 | 410, 432 | hsa-miR-199a | 3475 | 5 | 2.16e−011 |
| 4748 | 46 | 251 | 282, 495 | hsa-miR-19b | 1266 | 5 | 2.84e−011 |
| 4749 | 46 | 152 | 369 | hsa-miR-200a | 3365 | 5 | 6.87e−010 |
| 4750 | 46 | 157 | 411 | hsa-miR-200b | 5480 | 5 | 1.16e−009 |
| 4751 | 46 | 156 | 446 | hsa-miR-200c | 8096 | 5 | 4.18e−011 |
| 4752 | 46 | 175 | 462 | hsa-miR-21 | 11559 | 5 | 2.39e−010 |
| 4753 | 46 | 115 | 314 | hsa-miR-210 | 977 | 5 | 2.51e−010 |
| 4754 | 46 | 29 | 439 | hsa-miR-214 | 2196 | 5 | 1.32e−011 |
| 4755 | 46 | 18 | 322 | hsa-miR-22 | 4318 | 5 | 6.09e−011 |
| 4756 | 46 | 50 | 364 | hsa-miR-221 | 2604 | 5 | 1.62e−011 |
| 4757 | 46 | 49 | 302 | hsa-miR-222 | 3757 | 5 | 2.32e−011 |
| 4758 | 46 | 67 | 336 | hsa-miR-23a | 3445 | 5 | 1.59e−011 |
| 4759 | 46 | 66 | 306 | hsa-miR-23b | 2300 | 5 | 5.85e−009 |
| 4760 | 46 | 238 | 346, 416 | hsa-miR-24 | 9370 | 5 | 3.62e−012 |
| 4761 | 46 | 90 | 388 | hsa-miR-25 | 748 | 5 | 6.68e−012 |
| 4762 | 46 | 256 | 441, 487 | hsa-miR-26a | 3819 | 5 | 3.62e−011 |
| 4763 | 46 | 257 | 409 | hsa-miR-26b | 503 | 5 | 1.28e−011 |
| 4764 | 46 | 259 | 481 | hsa-miR-27a | 3473 | 5 | 2.23e−011 |
| 4765 | 46 | 260 | 280 | hsa-miR-27b | 1232 | 5 | 2.16e−010 |
| 4766 | 46 | 61 | 286 | hsa-miR-296 | 1727 | 5 | 6.82e−011 |
| 4767 | 46 | 167 | 556 | hsa-miR-29a | 1797 | 5 | 1.56e−010 |
| 4768 | 46 | 244 | 287 | hsa-miR-30a-5p | 1039 | 5 | 1.36e−012 |
| 4769 | 46 | 243 | 299, 316 | hsa-miR-30c | 655 | 5 | 1.21e−009 |
| 4770 | 46 | 241 | 550 | hsa-miR-30d | 1474 | 5 | 1.12e−011 |
| 4771 | 46 | 245 | 431 | hsa-miR-30e-5p | 1588 | 5 | 3.67e−010 |
| 4772 | 46 | 132 | 429 | hsa-miR-31 | 4855 | 5 | 2.22e−010 |
| 4773 | 46 | 1 | 359 | hsa-miR-320 | 6369 | 5 | 4.51e−011 |
| 4774 | 46 | 113 | 538 | hsa-miR-328 | 1163 | 5 | 3.06e−012 |
| 4775 | 46 | 141 | 437 | hsa-miR-33 | 1170 | 5 | 5.21e−012 |
| 4776 | 46 | 126 | 380 | hsa-miR-331 | 1072 | 5 | 3.98e−013 |
| 4777 | 46 | 185 | 492 | hsa-miR-335 | 1775 | 5 | 6.3e−011 |
| 4778 | 46 | 194 | 337 | hsa-miR-338 | 2726 | 5 | 8.88e−012 |
| 4779 | 46 | 208 | 477 | hsa-miR-342 | 596 | 5 | 7.16e−013 |
| 4780 | 46 | 237 | 480 | hsa-miR-34a | 2037 | 5 | 3.22e−011 |
| 4781 | 46 | 128 | 294 | hsa-miR-370 | 625 | 5 | 2.13e−012 |
| 4782 | 46 | 35 | 406 | hsa-miR-373* | 553 | 5 | 8.47e−012 |
| 4783 | 46 | 273 | 335 | hsa-miR-375 | 532 | 5 | 1.77e−010 |
| 4784 | 46 | 112 | 372 | hsa-miR-422b | 714 | 5 | 6.95e−011 |
| 4785 | 46 | 51 | 497 | hsa-miR-423 | 3002 | 5 | 1.16e−012 |
| 4786 | 46 | 158 | 378 | hsa-miR-429 | 639 | 5 | 2.52e−010 |
| 4787 | 46 | 236 | 413 | hsa-miR-449 | 3213 | 5 | 6.87e−011 |
| 4788 | 46 | 44 | 547 | hsa-miR-485-5p | 817 | 5 | 6.75e−012 |
| 4789 | 46 | 63 | 557 | hsa-miR-491 | 1869 | 5 | 3.14e−012 |
| 4790 | 46 | 54 | 293 | hsa-miR-492 | 546 | 5 | 1.02e−011 |
| 4791 | 46 | 214 | 376 | hsa-miR-494 | 21190 | 5 | 2.31e−010 |
| 4792 | 46 | 83 | 312 | hsa-miR-497 | 609 | 5 | 6.02e−012 |
| 4793 | 46 | 258 | 428, 443 | hsa-miR-513 | 710 | 5 | 1.09e−011 |
| 4794 | 46 | 103 | 342 | hsa-miR-525 | 539 | 5 | 6.3e−013 |
| 4795 | 46 | 135 | 315 | hsa-miR-565 | 1527 | 5 | 1.81e−011 |
| 4796 | 46 | 60 | 479 | hsa-miR-638 | 5806 | 5 | 1.15e−011 |
| 4797 | 46 | 195 | 421 | hsa-miR-662 | 616 | 5 | 1.06e−009 |
| 4798 | 46 | 59 | 330 | hsa-miR-663 | 2556 | 5 | 8.01e−012 |
| 4799 | 46 | 186 | 341 | hsa-miR-768-3p | 1388 | 5 | 6.16e−011 |
| 4800 | 46 | 123 | 291 | hsa-miR-801 | 818 | 5 | 1.66e−012 |
| 4801 | 46 | 182 | 304, 317 | hsa-miR-92 | 4621 | 5 | 8.1e−013 |
| 4802 | 46 | 6 | 471 | hsa-miR-93 | 2099 | 5 | 4.06e−011 |
| 4803 | 46 | 14 | 318 | hsa-miR-99a | 1308 | 5 | 1.99e−011 |
| 4804 | 46 | 80 | 509 | hsa-miR-99b | 630 | 5 | 2.34e−011 |
| 4805 | 47 | 127 | 361 | 11_1 | 760 | 5 | 1.17e−011 |
| 4806 | 47 | 196 | 508 | 15_rel | 55651 | 5 | 3.5e−013 |
| 4807 | 47 | 197 | 444 | 25_6 | 573 | 5 | 8.02e−012 |
| 4808 | 47 | 219 | 497 | 27_3 | 743 | 5 | 6.8e−015 |
| 4809 | 47 | 145 | 447 | 43_44_rel | 67698 | 5 | 1.38e−012 |
| 4810 | 47 | 183 | 373 | 66_1 | 616 | 5 | 4.73e−012 |
| 4811 | 47 | 94 | 394 | 69_6_rel | 6046 | 5 | 7.01e−012 |
| 4812 | 47 | 40 | 553 | 72_4 | 723 | 5 | 7.06e−012 |
| 4813 | 47 | 97 | 357 | 74_26_rel | 1091 | 5 | 1.94e−011 |
| 4814 | 47 | 33 | 339, 552 | 96_rel | 41649 | 5 | 3.85e−013 |
| 4815 | 47 | 87 | 301 | gam11 | 9806 | 5 | 2.43e−011 |
| 4816 | 47 | 223 | 320, 393, 559 | hsa-let-7a | 765 | 5 | 1.85e−012 |
| 4817 | 47 | 225 | 332 | hsa-let-7b | 1967 | 5 | 6.24e−013 |
| 4818 | 47 | 224 | 427 | hsa-let-7c | 864 | 5 | 4.73e−012 |
| 4819 | 47 | 45 | 277 | hsa-let-7d | 574 | 5 | 1.91e−012 |
| 4820 | 47 | 220 | 475 | hsa-let-7e | 1032 | 5 | 1.82e−012 |
| 4821 | 47 | 226 | 395 | hsa-let-7g | 573 | 5 | 2e−012 |
| 4822 | 47 | 227 | 297 | hsa-let-7i | 632 | 5 | 2.36e−012 |
| 4823 | 47 | 48 | 483, 529 | hsa-miR-103 | 1189 | 5 | 1.42e−011 |
| 4824 | 47 | 184 | 423 | hsa-miR-105 | 617 | 5 | 1.93e−012 |
| 4825 | 47 | 2 | 399 | hsa-miR-106a | 766 | 5 | 1e−012 |
| 4826 | 47 | 149 | 392 | hsa-miR-106b | 1050 | 5 | 2.55e−012 |
| 4827 | 47 | 47 | 555 | hsa-miR-107 | 1090 | 5 | 9.32e−012 |
| 4828 | 47 | 199 | 387 | hsa-miR-125a | 1183 | 5 | 3.13e−012 |
| 4829 | 47 | 198 | 313, 551 | hsa-miR-125b | 1635 | 5 | 3.95e−012 |
| 4830 | 47 | 204 | 419 | hsa-miR-126 | 1079 | 5 | 7.76e−012 |
| 4831 | 47 | 89 | 419 | hsa-miR-126* | 655 | 5 | 2.78e−012 |
| 4832 | 47 | 86 | 417 | hsa-miR-130a | 1536 | 5 | 1.02e−012 |
| 4833 | 47 | 52 | 370, 548 | hsa-miR-138 | 648 | 5 | 1.02e−011 |
| 4834 | 47 | 218 | 527 | hsa-miR-143 | 1723 | 5 | 3.09e−011 |
| 4835 | 47 | 139 | 281 | hsa-miR-145 | 1062 | 5 | 4.75e−012 |
| 4836 | 47 | 209 | 507 | hsa-miR-150 | 678 | 5 | 7.51e−011 |
| 4837 | 47 | 173 | 360, 468 | hsa-miR-16 | 1618 | 5 | 9.13e−012 |
| 4838 | 47 | 75 | 467 | hsa-miR-17-5p | 600 | 5 | 9.28e−014 |
| 4839 | 47 | 10 | 371, 461 | hsa-miR-181a | 696 | 5 | 2.81e−012 |
| 4840 | 47 | 272 | 323 | hsa-miR-182 | 1076 | 5 | 1.35e−010 |
| 4841 | 47 | 179 | 356 | hsa-miR-183 | 765 | 5 | 1.07e−011 |
| 4842 | 47 | 77 | 440 | hsa-miR-191 | 607 | 5 | 2.03e−012 |
| 4843 | 47 | 130 | 440 | hsa-miR-191* | 1021 | 5 | 1.18e−010 |
| 4844 | 47 | 17 | 442 | hsa-miR-193a | 1047 | 5 | 1.75e−010 |
| 4845 | 47 | 16 | 488 | hsa-miR-193b | 614 | 5 | 7.1e−012 |
| 4846 | 47 | 92 | 410, 432 | hsa-miR-199a | 1214 | 5 | 3.49e−011 |
| 4847 | 47 | 251 | 282, 495 | hsa-miR-19b | 1224 | 5 | 3.6e−012 |
| 4848 | 47 | 175 | 462 | hsa-miR-21 | 1414 | 5 | 2.75e−012 |
| 4849 | 47 | 159 | 494 | hsa-miR-216 | 577 | 5 | 4.86e−012 |
| 4850 | 47 | 18 | 322 | hsa-miR-22 | 930 | 5 | 6.06e−014 |
| 4851 | 47 | 49 | 302 | hsa-miR-222 | 701 | 5 | 8.85e−014 |
| 4852 | 47 | 67 | 336 | hsa-miR-23a | 815 | 5 | 3.65e−012 |
| 4853 | 47 | 66 | 306 | hsa-miR-23b | 1127 | 5 | 5.73e−012 |
| 4854 | 47 | 238 | 346, 416 | hsa-miR-24 | 2804 | 5 | 1.87e−012 |
| 4855 | 47 | 90 | 388 | hsa-miR-25 | 852 | 5 | 9.11e−012 |
| 4856 | 47 | 256 | 441, 487 | hsa-miR-26a | 2327 | 5 | 1.17e−012 |
| 4857 | 47 | 259 | 481 | hsa-miR-27a | 1643 | 5 | 1.69e−011 |
| 4858 | 47 | 61 | 286 | hsa-miR-296 | 2291 | 5 | 4.3e−011 |
| 4859 | 47 | 117 | 287 | hsa-miR-30a-3p | 542 | 5 | 1.35e−011 |
| 4860 | 47 | 1 | 359 | hsa-miR-320 | 2185 | 5 | 3.35e−012 |
| 4861 | 47 | 91 | 368 | hsa-miR-324-3p | 503 | 5 | 1.02e−012 |
| 4862 | 47 | 113 | 538 | hsa-miR-328 | 638 | 5 | 4.71e−012 |
| 4863 | 47 | 141 | 437 | hsa-miR-33 | 650 | 5 | 2.58e−012 |
| 4864 | 47 | 126 | 380 | hsa-miR-331 | 725 | 5 | 5.66e−013 |
| 4865 | 47 | 185 | 492 | hsa-miR-335 | 503 | 5 | 5.85e−013 |
| 4866 | 47 | 194 | 337 | hsa-miR-338 | 1222 | 5 | 1.82e−012 |
| 4867 | 47 | 208 | 477 | hsa-miR-342 | 531 | 5 | 8.7e−013 |
| 4868 | 47 | 237 | 480 | hsa-miR-34a | 1108 | 5 | 4.35e−012 |
| 4869 | 47 | 128 | 294 | hsa-miR-370 | 615 | 5 | 1.57e−013 |
| 4870 | 47 | 142 | 537 | hsa-miR-371 | 1150 | 5 | 3.87e−012 |
| 4871 | 47 | 5 | 433 | hsa-miR-372 | 7922 | 5 | 1.02e−011 |
| 4872 | 47 | 119 | 406 | hsa-miR-373 | 7568 | 5 | 2.59e−011 |
| 4873 | 47 | 35 | 406 | hsa-miR-373* | 813 | 5 | 1.58e−011 |
| 4874 | 47 | 112 | 372 | hsa-miR-422b | 590 | 5 | 1.53e−011 |
| 4875 | 47 | 51 | 497 | hsa-miR-423 | 1353 | 5 | 1.47e−012 |
| 4876 | 47 | 236 | 413 | hsa-miR-449 | 1477 | 5 | 2.84e−011 |
| 4877 | 47 | 44 | 547 | hsa-miR-485-5p | 543 | 5 | 1.35e−012 |
| 4878 | 47 | 63 | 557 | hsa-miR-491 | 4782 | 5 | 5.79e−011 |
| 4879 | 47 | 214 | 376 | hsa-miR-494 | 7227 | 5 | 6.16e−011 |
| 4880 | 47 | 270 | 390 | hsa-miR-498 | 506 | 5 | 2.31e−011 |
| 4881 | 47 | 43 | 418 | hsa-miR-504 | 568 | 5 | 8.06e−014 |
| 4882 | 47 | 135 | 315 | hsa-miR-565 | 876 | 5 | 2.83e−012 |
| 4883 | 47 | 60 | 479 | hsa-miR-638 | 4654 | 5 | 1.36e−011 |
| 4884 | 47 | 195 | 421 | hsa-miR-662 | 735 | 5 | 2.03e−012 |
| 4885 | 47 | 59 | 330 | hsa-miR-663 | 3919 | 5 | 2.26e−011 |
| 4886 | 47 | 36 | 385 | hsa-miR-766 | 541 | 5 | 2.78e−010 |
| 4887 | 47 | 186 | 341 | hsa-miR-768-3p | 1790 | 5 | 3.51e−011 |
| 4888 | 47 | 123 | 291 | hsa-miR-801 | 1705 | 5 | 9.65e−011 |
| 4889 | 47 | 182 | 304, 317 | hsa-miR-92 | 2232 | 5 | 9.91e−012 |
| 4890 | 47 | 6 | 471 | hsa-miR-93 | 977 | 5 | 1.76e−011 |
| 4891 | 48 | 127 | 361 | 11_1 | 3274 | 5 | 2.93e−009 |
| 4892 | 48 | 196 | 508 | 15_rel | 64740 | 5 | 1.77e−010 |
| 4893 | 48 | 197 | 444 | 25_6 | 863 | 5 | 3.58e−010 |
| 4894 | 48 | 219 | 497 | 27_3 | 2597 | 5 | 1.36e−009 |
| 4895 | 48 | 129 | 548 | 43_39 | 1007 | 5 | 1.5e−011 |
| 4896 | 48 | 27 | 313 | 43_40 | 1662 | 5 | 1.51e−009 |
| 4897 | 48 | 145 | 447 | 43_44_rel | 64755 | 5 | 1.77e−010 |
| 4898 | 48 | 183 | 373 | 66_1 | 718 | 5 | 3.72e−010 |
| 4899 | 48 | 94 | 394 | 69_6_rel | 7955 | 5 | 2.77e−011 |
| 4900 | 48 | 121 | 502 | 70_14 | 1231 | 5 | 5.4e−009 |
| 4901 | 48 | 177 | 366, 464 | 72_12 | 7666 | 5 | 4.13e−010 |
| 4902 | 48 | 40 | 553 | 72_4 | 2305 | 5 | 4.55e−009 |
| 4903 | 48 | 97 | 357 | 74_26_rel | 2782 | 5 | 5.61e−012 |
| 4904 | 48 | 33 | 339, 552 | 96_rel | 59454 | 5 | 8.99e−010 |
| 4905 | 48 | 87 | 301 | gam11 | 18053 | 5 | 6.08e−011 |
| 4906 | 48 | 125 | 532 | 70_16 | 690 | 5 | 1.39e−011 |
| 4907 | 48 | 163 | 402 | 43_7 | 655 | 5 | 6.34e−011 |
| 4908 | 48 | 223 | 320, 393, 559 | hsa-let-7a | 27054 | 5 | 3.46e−008 |
| 4909 | 48 | 223 | 320, 393, 559 | hsa-let-7a | 9596 | 5 | 5.02e−008 |
| 4910 | 48 | 225 | 332 | hsa-let-7b | 33987 | 5 | 5.05e−010 |
| 4911 | 48 | 224 | 427 | hsa-let-7c | 27799 | 5 | 2.49e−009 |
| 4912 | 48 | 45 | 277 | hsa-let-7d | 12234 | 5 | 1.95e−010 |
| 4913 | 48 | 220 | 475 | hsa-let-7e | 6680 | 5 | 3.8e−010 |
| 4914 | 48 | 222 | 482, 558 | hsa-let-7f | 1713 | 5 | 2.29e−008 |
| 4915 | 48 | 226 | 395 | hsa-let-7g | 2573 | 5 | 1.18e−009 |
| 4916 | 48 | 227 | 297 | hsa-let-7i | 8684 | 5 | 8.5e−009 |
| 4917 | 48 | 13 | 397 | hsa-miR-100 | 10462 | 5 | 1.66e−009 |
| 4918 | 48 | 48 | 483, 529 | hsa-miR-103 | 8259 | 5 | 9.09e−010 |
| 4919 | 48 | 184 | 423 | hsa-miR-105 | 2751 | 5 | 6.7e−011 |
| 4920 | 48 | 2 | 399 | hsa-miR-106a | 2928 | 5 | 7.75e−011 |
| 4921 | 48 | 149 | 392 | hsa-miR-106b | 2242 | 5 | 1.57e−009 |
| 4922 | 48 | 47 | 555 | hsa-miR-107 | 7303 | 5 | 8.2e−010 |
| 4923 | 48 | 199 | 387 | hsa-miR-125a | 18071 | 5 | 1.04e−009 |
| 4924 | 48 | 198 | 313, 551 | hsa-miR-125b | 54823 | 5 | 8.26e−010 |
| 4925 | 48 | 204 | 419 | hsa-miR-126 | 14723 | 5 | 7.89e−009 |
| 4926 | 48 | 89 | 419 | hsa-miR-126* | 915 | 5 | 8.1e−011 |
| 4927 | 48 | 86 | 417 | hsa-miR-130a | 6306 | 5 | 2.34e−009 |
| 4928 | 48 | 85 | 328 | hsa-miR-130b | 581 | 5 | 4.44e−010 |
| 4929 | 48 | 266 | 295, 386 | hsa-miR-133a | 732 | 5 | 1.04e−009 |
| 4930 | 48 | 265 | 533 | hsa-miR-133b | 501 | 5 | 2.17e−009 |
| 4931 | 48 | 52 | 370, 548 | hsa-miR-138 | 1589 | 5 | 3.9e−009 |
| 4932 | 48 | 64 | 402 | hsa-miR-140 | 664 | 5 | 5.7e−011 |
| 4933 | 48 | 151 | 472 | hsa-miR-141 | 8935 | 5 | 7.38e−011 |
| 4934 | 48 | 218 | 527 | hsa-miR-143 | 10807 | 5 | 1.57e−009 |
| 4935 | 48 | 139 | 281 | hsa-miR-145 | 10152 | 5 | 1.17e−009 |
| 4936 | 48 | 190 | 458 | hsa-miR-148a | 618 | 5 | 1.04e−007 |
| 4937 | 48 | 191 | 289 | hsa-miR-148b | 813 | 5 | 9.95e−008 |
| 4938 | 48 | 212 | 333 | hsa-miR-149 | 741 | 5 | 2.03e−011 |
| 4939 | 48 | 209 | 507 | hsa-miR-150 | 1095 | 5 | 5.81e−009 |
| 4940 | 48 | 34 | 396 | hsa-miR-151 | 902 | 5 | 1.73e−010 |
| 4941 | 48 | 192 | 470 | hsa-miR-152 | 1249 | 5 | 1.5e−009 |
| 4942 | 48 | 171 | 331 | hsa-miR-15a | 507 | 5 | 3.71e−008 |
| 4943 | 48 | 172 | 329 | hsa-miR-15b | 2290 | 5 | 3.5e−008 |
| 4944 | 48 | 173 | 360, 468 | hsa-miR-16 | 11230 | 5 | 1.1e−008 |
| 4945 | 48 | 38 | 467 | hsa-miR-17-3p | 1288 | 5 | 7.06e−011 |
| 4946 | 48 | 75 | 467 | hsa-miR-17-5p | 2212 | 5 | 6.6e−011 |
| 4947 | 48 | 10 | 371, 461 | hsa-miR-181a | 8680 | 5 | 1.4e−009 |
| 4948 | 48 | 11 | 456, 540 | hsa-miR-181b | 2194 | 5 | 6.94e−009 |
| 4949 | 48 | 9 | 451 | hsa-miR-181c | 549 | 5 | 4.23e−008 |
| 4950 | 48 | 179 | 356 | hsa-miR-183 | 796 | 5 | 1.39e−010 |
| 4951 | 48 | 234 | 450 | hsa-miR-185 | 2032 | 5 | 1.02e−009 |
| 4952 | 48 | 144 | 346 | hsa-miR-189 | 631 | 5 | 1.93e−011 |
| 4953 | 48 | 77 | 440 | hsa-miR-191 | 5201 | 5 | 1.83e−010 |
| 4954 | 48 | 130 | 440 | hsa-miR-191* | 989 | 5 | 4.09e−009 |
| 4955 | 48 | 109 | 374 | hsa-miR-192 | 589 | 5 | 9.35e−012 |
| 4956 | 48 | 17 | 442 | hsa-miR-193a | 9886 | 5 | 4.88e−011 |
| 4957 | 48 | 16 | 488 | hsa-miR-193b | 923 | 5 | 1.21e−009 |
| 4958 | 48 | 246 | 290, 549 | hsa-miR-194 | 523 | 5 | 3.49e−009 |
| 4959 | 48 | 170 | 327 | hsa-miR-195 | 1094 | 5 | 1.75e−007 |
| 4960 | 48 | 92 | 410, 432 | hsa-miR-199a | 9187 | 5 | 1.15e−010 |
| 4961 | 48 | 162 | 410, 432, 473 | hsa-miR-199a* | 1768 | 5 | 6.4e−008 |
| 4962 | 48 | 93 | 473 | hsa-miR-199b | 554 | 5 | 3.39e−008 |
| 4963 | 48 | 251 | 282, 495 | hsa-miR-19b | 3086 | 5 | 4.27e−009 |
| 4964 | 48 | 152 | 369 | hsa-miR-200a | 2367 | 5 | 2.5e−009 |
| 4965 | 48 | 157 | 411 | hsa-miR-200b | 3436 | 5 | 8.25e−009 |
| 4966 | 48 | 156 | 446 | hsa-miR-200c | 13063 | 5 | 1.15e−011 |
| 4967 | 48 | 203 | 504 | hsa-miR-205 | 536 | 5 | 6.51e−009 |
| 4968 | 48 | 175 | 462 | hsa-miR-21 | 6560 | 5 | 1.17e−008 |
| 4969 | 48 | 29 | 439 | hsa-miR-214 | 2916 | 5 | 7.43e−010 |
| 4970 | 48 | 159 | 494 | hsa-miR-216 | 572 | 5 | 2.66e−010 |
| 4971 | 48 | 18 | 322 | hsa-miR-22 | 6630 | 5 | 1.13e−009 |
| 4972 | 48 | 50 | 364 | hsa-miR-221 | 9742 | 5 | 1.79e−007 |
| 4973 | 48 | 49 | 302 | hsa-miR-222 | 12844 | 5 | 1.99e−007 |
| 4974 | 48 | 67 | 336 | hsa-miR-23a | 11825 | 5 | 9.87e−011 |
| 4975 | 48 | 66 | 306 | hsa-miR-23b | 20700 | 5 | 6.8e−011 |
| 4976 | 48 | 238 | 346, 416 | hsa-miR-24 | 42007 | 5 | 4.8e−012 |
| 4977 | 48 | 90 | 388 | hsa-miR-25 | 933 | 5 | 1.38e−009 |
| 4978 | 48 | 256 | 441, 487 | hsa-miR-26a | 33347 | 5 | 4.87e−009 |
| 4979 | 48 | 257 | 409 | hsa-miR-26b | 877 | 5 | 2.39e−009 |
| 4980 | 48 | 259 | 481 | hsa-miR-27a | 11459 | 5 | 3.7e−009 |
| 4981 | 48 | 260 | 280 | hsa-miR-27b | 4804 | 5 | 1.76e−008 |
| 4982 | 48 | 61 | 286 | hsa-miR-296 | 1983 | 5 | 3.34e−009 |
| 4983 | 48 | 167 | 556 | hsa-miR-29a | 10444 | 5 | 1.96e−008 |
| 4984 | 48 | 168 | 303, 530 | hsa-miR-29b | 1732 | 5 | 2.35e−008 |
| 4985 | 48 | 169 | 528 | hsa-miR-29c | 634 | 5 | 9.12e−008 |
| 4986 | 48 | 117 | 287 | hsa-miR-30a-3p | 1124 | 5 | 2.08e−010 |
| 4987 | 48 | 244 | 287 | hsa-miR-30a-5p | 12203 | 5 | 4.44e−010 |
| 4988 | 48 | 242 | 311 | hsa-miR-30b | 2908 | 5 | 6.51e−008 |
| 4989 | 48 | 243 | 299, 316 | hsa-miR-30c | 9019 | 5 | 5.54e−008 |
| 4990 | 48 | 241 | 550 | hsa-miR-30d | 4570 | 5 | 1.22e−007 |
| 4991 | 48 | 116 | 431 | hsa-miR-30e-3p | 671 | 5 | 6.91e−010 |
| 4992 | 48 | 245 | 431 | hsa-miR-30e-5p | 3599 | 5 | 8.43e−007 |
| 4993 | 48 | 132 | 429 | hsa-miR-31 | 8786 | 5 | 2.36e−008 |
| 4994 | 48 | 1 | 359 | hsa-miR-320 | 9279 | 5 | 6.63e−010 |
| 4995 | 48 | 91 | 368 | hsa-miR-324-3p | 2187 | 5 | 4.99e−010 |
| 4996 | 48 | 98 | 368 | hsa-miR-324-5p | 873 | 5 | 1.28e−008 |
| 4997 | 48 | 113 | 538 | hsa-miR-328 | 2453 | 5 | 9.43e−010 |
| 4998 | 48 | 141 | 437 | hsa-miR-33 | 3622 | 5 | 6.85e−010 |
| 4999 | 48 | 126 | 380 | hsa-miR-331 | 5344 | 5 | 2.25e−010 |
| 5000 | 48 | 185 | 492 | hsa-miR-335 | 3403 | 5 | 3.08e−008 |
| 5001 | 48 | 194 | 337 | hsa-miR-338 | 4851 | 5 | 1.35e−009 |
| 5002 | 48 | 201 | 463 | hsa-miR-340 | 612 | 5 | 2.17e−010 |
| 5003 | 48 | 208 | 477 | hsa-miR-342 | 1523 | 5 | 1.86e−009 |
| 5004 | 48 | 229 | 292 | hsa-miR-345 | 3388 | 5 | 5.85e−010 |
| 5005 | 48 | 237 | 480 | hsa-miR-34a | 3154 | 5 | 1.09e−009 |
| 5006 | 48 | 255 | 444 | hsa-miR-361 | 1086 | 5 | 4.23e−012 |
| 5007 | 48 | 160 | 379, 382 | hsa-miR-365 | 689 | 5 | 1.38e−011 |
| 5008 | 48 | 128 | 294 | hsa-miR-370 | 959 | 5 | 2.2e−009 |
| 5009 | 48 | 35 | 406 | hsa-miR-373* | 691 | 5 | 3.68e−010 |
| 5010 | 48 | 112 | 372 | hsa-miR-422b | 687 | 5 | 1e−010 |
| 5011 | 48 | 51 | 497 | hsa-miR-423 | 4641 | 5 | 8.32e−010 |
| 5012 | 48 | 236 | 413 | hsa-miR-449 | 2444 | 5 | 1.28e−010 |
| 5013 | 48 | 4 | 430 | hsa-miR-451 | 3368 | 5 | 3.51e−007 |
| 5014 | 48 | 252 | 405 | hsa-miR-452 | 654 | 5 | 3.55e−010 |
| 5015 | 48 | 44 | 547 | hsa-miR-485-5p | 890 | 5 | 1.54e−009 |
| 5016 | 48 | 202 | 350 | hsa-miR-486 | 910 | 5 | 3.25e−009 |
| 5017 | 48 | 63 | 557 | hsa-miR-491 | 3129 | 5 | 3.14e−010 |
| 5018 | 48 | 54 | 293 | hsa-miR-492 | 574 | 5 | 4.29e−010 |
| 5019 | 48 | 267 | 354 | hsa-miR-493-5p | 532 | 5 | 7.41e−009 |
| 5020 | 48 | 214 | 376 | hsa-miR-494 | 24979 | 5 | 6.22e−009 |
| 5021 | 48 | 83 | 312 | hsa-miR-497 | 1751 | 5 | 1.37e−009 |
| 5022 | 48 | 174 | 534 | hsa-miR-503 | 531 | 5 | 5.58e−010 |
| 5023 | 48 | 43 | 418 | hsa-miR-504 | 907 | 5 | 1.23e−009 |
| 5024 | 48 | 258 | 428, 443 | hsa-miR-513 | 842 | 5 | 2.25e−010 |
| 5025 | 48 | 210 | 400 | hsa-miR-518c* | 742 | 5 | 8.69e−010 |
| 5026 | 48 | 102 | 344 | hsa-miR-520a* | 824 | 5 | 5.47e−010 |
| 5027 | 48 | 206 | 513 | hsa-miR-520d* | 678 | 5 | 1.06e−009 |
| 5028 | 48 | 15 | 526 | hsa-miR-523 | 615 | 5 | 1.76e−008 |
| 5029 | 48 | 100 | 524 | hsa-miR-524* | 584 | 5 | 5.84e−010 |
| 5030 | 48 | 103 | 342 | hsa-miR-525 | 837 | 5 | 9.45e−010 |
| 5031 | 48 | 108 | 501 | hsa-miR-526b | 669 | 5 | 8.16e−010 |
| 5032 | 48 | 135 | 315 | hsa-miR-565 | 3720 | 5 | 4.35e−009 |
| 5033 | 48 | 60 | 479 | hsa-miR-638 | 7082 | 5 | 2.63e−011 |
| 5034 | 48 | 195 | 421 | hsa-miR-662 | 1843 | 5 | 9.41e−010 |
| 5035 | 48 | 59 | 330 | hsa-miR-663 | 1920 | 5 | 2.68e−010 |
| 5036 | 48 | 186 | 341 | hsa-miR-768-3p | 13484 | 5 | 4.51e−010 |
| 5037 | 48 | 147 | 341 | hsa-miR-768-5p | 2356 | 5 | 6.01e−010 |
| 5038 | 48 | 123 | 291 | hsa-miR-801 | 2358 | 5 | 2.49e−010 |
| 5039 | 48 | 182 | 304, 317 | hsa-miR-92 | 4588 | 5 | 8.7e−009 |
| 5040 | 48 | 6 | 471 | hsa-miR-93 | 2409 | 5 | 9.66e−012 |
| 5041 | 48 | 14 | 318 | hsa-miR-99a | 13776 | 5 | 2.23e−010 |
| 5042 | 48 | 80 | 509 | hsa-miR-99b | 2556 | 5 | 1.38e−009 |
| 5043 | 48 | 131 | 430 | 6_11 | 4088 | 5 | 1.18e−007 |
| 5044 | 49 | 127 | 361 | 11_1 | 1837 | 10 | 1.32e−022 |
| 5045 | 49 | 196 | 508 | 15_rel | 39456 | 10 | 2.37e−022 |
| 5046 | 49 | 197 | 444 | 25_6 | 584 | 10 | 2.81e−023 |
| 5047 | 49 | 219 | 497 | 27_3 | 1128 | 10 | 1e−023 |
| 5048 | 49 | 27 | 313 | 43_40 | 637 | 10 | 2.05e−022 |
| 5049 | 49 | 145 | 447 | 43_44_rel | 55273 | 10 | 1.89e−023 |
| 5050 | 49 | 94 | 394 | 69_6_rel | 3721 | 10 | 1.53e−021 |
| 5051 | 49 | 177 | 366, 464 | 72_12 | 3517 | 10 | 8.86e−022 |
| 5052 | 49 | 40 | 553 | 72_4 | 1336 | 10 | 5.82e−022 |
| 5053 | 49 | 97 | 357 | 74_26_rel | 802 | 10 | 1.33e−021 |
| 5054 | 49 | 33 | 339, 552 | 96_rel | 36089 | 10 | 1.13e−022 |
| 5055 | 49 | 87 | 301 | gam11 | 7544 | 10 | 8.67e−021 |
| 5056 | 49 | 223 | 320, 393, 559 | hsa-let-7a | 5827 | 10 | 5.18e−020 |
| 5057 | 49 | 223 | 320, 393, 559 | hsa-let-7a | 2295 | 10 | 5.36e−019 |
| 5058 | 49 | 225 | 332 | hsa-let-7b | 12844 | 10 | 5.16e−024 |
| 5059 | 49 | 224 | 427 | hsa-let-7c | 9035 | 10 | 1.07e−023 |
| 5060 | 49 | 45 | 277 | hsa-let-7d | 3332 | 10 | 1.27e−022 |
| 5061 | 49 | 220 | 475 | hsa-let-7e | 3009 | 10 | 4.1e−025 |
| 5062 | 49 | 226 | 395 | hsa-let-7g | 876 | 10 | 2.64e−024 |
| 5063 | 49 | 227 | 297 | hsa-let-7i | 3521 | 10 | 1.73e−020 |
| 5064 | 49 | 13 | 397 | hsa-miR-100 | 3149 | 10 | 3.71e−022 |
| 5065 | 49 | 48 | 483, 529 | hsa-miR-103 | 3453 | 10 | 4.49e−021 |
| 5066 | 49 | 184 | 423 | hsa-miR-105 | 1075 | 10 | 3.88e−023 |
| 5067 | 49 | 2 | 399 | hsa-miR-106a | 1130 | 10 | 6.72e−023 |
| 5068 | 49 | 149 | 392 | hsa-miR-106b | 1122 | 10 | 1.02e−024 |
| 5069 | 49 | 47 | 555 | hsa-miR-107 | 2916 | 10 | 7.81e−022 |
| 5070 | 49 | 199 | 387 | hsa-miR-125a | 7241 | 10 | 2.8e−024 |
| 5071 | 49 | 198 | 313, 551 | hsa-miR-125b | 30817 | 10 | 4.28e−024 |
| 5072 | 49 | 204 | 419 | hsa-miR-126 | 4570 | 10 | 4.96e−021 |
| 5073 | 49 | 86 | 417 | hsa-miR-130a | 2051 | 10 | 2.07e−024 |
| 5074 | 49 | 52 | 370, 548 | hsa-miR-138 | 515 | 10 | 1.22e−024 |
| 5075 | 49 | 151 | 472 | hsa-miR-141 | 4183 | 10 | 2.88e−022 |
| 5076 | 49 | 218 | 527 | hsa-miR-143 | 4887 | 10 | 4.81e−023 |
| 5077 | 49 | 139 | 281 | hsa-miR-145 | 3794 | 10 | 8.03e−021 |
| 5078 | 49 | 216 | 326 | hsa-miR-146b | 928 | 10 | 1.76e−016 |
| 5079 | 49 | 209 | 507 | hsa-miR-150 | 517 | 10 | 1.15e−021 |
| 5080 | 49 | 172 | 329 | hsa-miR-15b | 852 | 10 | 1.53e−020 |
| 5081 | 49 | 173 | 360, 468 | hsa-miR-16 | 4568 | 10 | 2.05e−020 |
| 5082 | 49 | 75 | 467 | hsa-miR-17-5p | 963 | 10 | 6.9e−023 |
| 5083 | 49 | 10 | 371, 461 | hsa-miR-181a | 4229 | 10 | 7.61e−024 |
| 5084 | 49 | 11 | 456, 540 | hsa-miR-181b | 1057 | 10 | 2.71e−024 |
| 5085 | 49 | 234 | 450 | hsa-miR-185 | 868 | 10 | 3.74e−024 |
| 5086 | 49 | 77 | 440 | hsa-miR-191 | 1910 | 10 | 1.79e−023 |
| 5087 | 49 | 130 | 440 | hsa-miR-191* | 1091 | 10 | 2.86e−022 |
| 5088 | 49 | 17 | 442 | hsa-miR-193a | 1851 | 10 | 6.99e−021 |
| 5089 | 49 | 16 | 488 | hsa-miR-193b | 553 | 10 | 3.8e−022 |
| 5090 | 49 | 92 | 410, 432 | hsa-miR-199a | 4039 | 10 | 3.4e−021 |
| 5091 | 49 | 162 | 410, 432, 473 | hsa-miR-199a* | 588 | 10 | 6.04e−021 |
| 5092 | 49 | 251 | 282, 495 | hsa-miR-19b | 1282 | 10 | 6.63e−022 |
| 5093 | 49 | 152 | 369 | hsa-miR-200a | 1129 | 10 | 4.2e−021 |
| 5094 | 49 | 157 | 411 | hsa-miR-200b | 1662 | 10 | 2.86e−021 |
| 5095 | 49 | 156 | 446 | hsa-miR-200c | 5650 | 10 | 3.81e−024 |
| 5096 | 49 | 175 | 462 | hsa-miR-21 | 4217 | 10 | 3.45e−018 |
| 5097 | 49 | 29 | 439 | hsa-miR-214 | 1209 | 10 | 3.52e−021 |
| 5098 | 49 | 18 | 322 | hsa-miR-22 | 3981 | 10 | 2.06e−022 |
| 5099 | 49 | 50 | 364 | hsa-miR-221 | 11093 | 10 | 5.64e−019 |
| 5100 | 49 | 49 | 302 | hsa-miR-222 | 13393 | 10 | 1.15e−019 |
| 5101 | 49 | 67 | 336 | hsa-miR-23a | 4630 | 10 | 2.12e−024 |
| 5102 | 49 | 66 | 306 | hsa-miR-23b | 7765 | 10 | 1.83e−022 |
| 5103 | 49 | 238 | 346, 416 | hsa-miR-24 | 16940 | 10 | 1.54e−023 |
| 5104 | 49 | 256 | 441, 487 | hsa-miR-26a | 11444 | 10 | 6.95e−022 |
| 5105 | 49 | 259 | 481 | hsa-miR-27a | 5706 | 10 | 5.21e−022 |
| 5106 | 49 | 260 | 280 | hsa-miR-27b | 2037 | 10 | 4.8e−020 |
| 5107 | 49 | 61 | 286 | hsa-miR-296 | 1735 | 10 | 4.6e−022 |
| 5108 | 49 | 167 | 556 | hsa-miR-29a | 4262 | 10 | 7.13e−021 |
| 5109 | 49 | 168 | 303, 530 | hsa-miR-29b | 506 | 10 | 1.02e−019 |
| 5110 | 49 | 117 | 287 | hsa-miR-30a-3p | 503 | 10 | 3.35e−025 |
| 5111 | 49 | 244 | 287 | hsa-miR-30a-5p | 3538 | 10 | 2.42e−023 |
| 5112 | 49 | 242 | 311 | hsa-miR-30b | 955 | 10 | 2.43e−020 |
| 5113 | 49 | 243 | 299, 316 | hsa-miR-30c | 2310 | 10 | 3.25e−020 |
| 5114 | 49 | 241 | 550 | hsa-miR-30d | 2100 | 10 | 5.19e−021 |
| 5115 | 49 | 245 | 431 | hsa-miR-30e-5p | 1111 | 10 | 6.34e−018 |
| 5116 | 49 | 132 | 429 | hsa-miR-31 | 5919 | 10 | 9.92e−021 |
| 5117 | 49 | 1 | 359 | hsa-miR-320 | 3770 | 10 | 6.44e−022 |
| 5118 | 49 | 91 | 368 | hsa-miR-324-3p | 986 | 10 | 2.57e−023 |
| 5119 | 49 | 113 | 538 | hsa-miR-328 | 1043 | 10 | 5.99e−024 |
| 5120 | 49 | 141 | 437 | hsa-miR-33 | 1415 | 10 | 6.35e−025 |
| 5121 | 49 | 126 | 380 | hsa-miR-331 | 1968 | 10 | 1.41e−023 |
| 5122 | 49 | 185 | 492 | hsa-miR-335 | 2259 | 10 | 4.08e−020 |
| 5123 | 49 | 194 | 337 | hsa-miR-338 | 2231 | 10 | 5.11e−022 |
| 5124 | 49 | 208 | 477 | hsa-miR-342 | 826 | 10 | 3.46e−025 |
| 5125 | 49 | 229 | 292 | hsa-miR-345 | 1291 | 10 | 7.61e−020 |
| 5126 | 49 | 237 | 480 | hsa-miR-34a | 2427 | 10 | 1.73e−020 |
| 5127 | 49 | 255 | 444 | hsa-miR-361 | 540 | 10 | 1.83e−024 |
| 5128 | 49 | 128 | 294 | hsa-miR-370 | 594 | 10 | 8.07e−022 |
| 5129 | 49 | 51 | 497 | hsa-miR-423 | 2227 | 10 | 2.69e−024 |
| 5130 | 49 | 236 | 413 | hsa-miR-449 | 1112 | 10 | 5.45e−021 |
| 5131 | 49 | 63 | 557 | hsa-miR-491 | 1632 | 10 | 6.14e−021 |
| 5132 | 49 | 214 | 376 | hsa-miR-494 | 8295 | 10 | 3.71e−020 |
| 5133 | 49 | 83 | 312 | hsa-miR-497 | 753 | 10 | 5.67e−023 |
| 5134 | 49 | 135 | 315 | hsa-miR-565 | 1786 | 10 | 3.22e−022 |
| 5135 | 49 | 60 | 479 | hsa-miR-638 | 3437 | 10 | 4.74e−022 |
| 5136 | 49 | 195 | 421 | hsa-miR-662 | 837 | 10 | 1.11e−022 |
| 5137 | 49 | 59 | 330 | hsa-miR-663 | 962 | 10 | 4.67e−023 |
| 5138 | 49 | 36 | 385 | hsa-miR-766 | 623 | 10 | 6.98e−021 |
| 5139 | 49 | 186 | 341 | hsa-miR-768-3p | 4594 | 10 | 6.86e−023 |
| 5140 | 49 | 147 | 341 | hsa-miR-768-5p | 943 | 10 | 3.86e−022 |
| 5141 | 49 | 123 | 291 | hsa-miR-801 | 1339 | 10 | 6.73e−023 |
| 5142 | 49 | 182 | 304, 317 | hsa-miR-92 | 2646 | 10 | 3e−022 |
| 5143 | 49 | 6 | 471 | hsa-miR-93 | 1247 | 10 | 3.17e−026 |
| 5144 | 49 | 14 | 318 | hsa-miR-99a | 4786 | 10 | 3.21e−023 |
| 5145 | 49 | 80 | 509 | hsa-miR-99b | 1199 | 10 | 1.07e−023 |
| 5146 | 49 | 131 | 430 | 6_11 | 751 | 10 | 5.06e−020 |
| 5147 | 50 | 127 | 361 | 11_1 | 820 | 6 | 1.08e−009 |
| 5148 | 50 | 196 | 508 | 15_rel | 32751 | 6 | 1.02e−007 |
| 5149 | 50 | 27 | 313 | 43_40 | 663 | 6 | 3.97e−013 |
| 5150 | 50 | 94 | 394 | 69_6_rel | 1062 | 6 | 3.71e−010 |
| 5151 | 50 | 177 | 366, 464 | 72_12 | 1765 | 6 | 1.36e−010 |
| 5152 | 50 | 97 | 357 | 74_26_rel | 599 | 6 | 1.43e−009 |
| 5153 | 50 | 87 | 301 | gam11 | 6426 | 6 | 6.21e−010 |
| 5154 | 50 | 125 | 532 | 70_16 | 658 | 6 | 1.79e−012 |
| 5155 | 50 | 163 | 402 | 43_7 | 1147 | 6 | 2.87e−010 |
| 5156 | 50 | 223 | 320, 393, 559 | hsa-let-7a | 12823 | 6 | 1.27e−010 |
| 5157 | 50 | 225 | 332 | hsa-let-7b | 40877 | 6 | 5.49e−011 |
| 5158 | 50 | 224 | 427 | hsa-let-7c | 22000 | 6 | 1.06e−011 |
| 5159 | 50 | 45 | 277 | hsa-let-7d | 4583 | 6 | 2.76e−011 |
| 5160 | 50 | 220 | 475 | hsa-let-7e | 3082 | 6 | 2.67e−011 |
| 5161 | 50 | 222 | 482, 558 | hsa-let-7f | 513 | 6 | 7.71e−012 |
| 5162 | 50 | 227 | 297 | hsa-let-7i | 1417 | 6 | 8.59e−012 |
| 5163 | 50 | 13 | 397 | hsa-miR-100 | 3571 | 6 | 5.8e−012 |
| 5164 | 50 | 48 | 483, 529 | hsa-miR-103 | 3361 | 6 | 2.74e−012 |
| 5165 | 50 | 184 | 423 | hsa-miR-105 | 926 | 6 | 7.7e−012 |
| 5166 | 50 | 2 | 399 | hsa-miR-106a | 1087 | 6 | 2.57e−011 |
| 5167 | 50 | 149 | 392 | hsa-miR-106b | 573 | 6 | 2.61e−012 |
| 5168 | 50 | 47 | 555 | hsa-miR-107 | 3154 | 6 | 2.41e−012 |
| 5169 | 50 | 165 | 340 | hsa-miR-10b | 1075 | 6 | 4.93e−010 |
| 5170 | 50 | 199 | 387 | hsa-miR-125a | 8849 | 6 | 7.33e−012 |
| 5171 | 50 | 198 | 313, 551 | hsa-miR-125b | 35736 | 6 | 4.56e−012 |
| 5172 | 50 | 204 | 419 | hsa-miR-126 | 2766 | 6 | 1.75e−009 |
| 5173 | 50 | 86 | 417 | hsa-miR-130a | 3027 | 6 | 3.81e−011 |
| 5174 | 50 | 218 | 527 | hsa-miR-143 | 36332 | 6 | 3.96e−012 |
| 5175 | 50 | 139 | 281 | hsa-miR-145 | 37837 | 6 | 6.34e−012 |
| 5176 | 50 | 212 | 333 | hsa-miR-149 | 974 | 6 | 2.71e−010 |
| 5177 | 50 | 34 | 396 | hsa-miR-151 | 528 | 6 | 1.01e−013 |
| 5178 | 50 | 172 | 329 | hsa-miR-15b | 693 | 6 | 3.64e−012 |
| 5179 | 50 | 173 | 360, 468 | hsa-miR-16 | 2717 | 6 | 8.01e−011 |
| 5180 | 50 | 38 | 467 | hsa-miR-17-3p | 585 | 6 | 1.56e−013 |
| 5181 | 50 | 75 | 467 | hsa-miR-17-5p | 609 | 6 | 2.41e−010 |
| 5182 | 50 | 10 | 371, 461 | hsa-miR-181a | 834 | 6 | 4.19e−010 |
| 5183 | 50 | 234 | 450 | hsa-miR-185 | 650 | 6 | 3.21e−010 |
| 5184 | 50 | 77 | 440 | hsa-miR-191 | 1586 | 6 | 1.16e−010 |
| 5185 | 50 | 17 | 442 | hsa-miR-193a | 4833 | 6 | 1.6e−011 |
| 5186 | 50 | 16 | 488 | hsa-miR-193b | 1156 | 6 | 1.92e−012 |
| 5187 | 50 | 170 | 327 | hsa-miR-195 | 2984 | 6 | 2.34e−010 |
| 5188 | 50 | 92 | 410, 432 | hsa-miR-199a | 8406 | 6 | 3.36e−010 |
| 5189 | 50 | 162 | 410, 432, 473 | hsa-miR-199a* | 1870 | 6 | 1.18e−010 |
| 5190 | 50 | 251 | 282, 495 | hsa-miR-19b | 723 | 6 | 3.29e−010 |
| 5191 | 50 | 175 | 462 | hsa-miR-21 | 2883 | 6 | 2.55e−009 |
| 5192 | 50 | 29 | 439 | hsa-miR-214 | 1855 | 6 | 3.7e−012 |
| 5193 | 50 | 18 | 322 | hsa-miR-22 | 1910 | 6 | 5.22e−010 |
| 5194 | 50 | 50 | 364 | hsa-miR-221 | 3636 | 6 | 3.43e−014 |
| 5195 | 50 | 49 | 302 | hsa-miR-222 | 5561 | 6 | 1.91e−013 |
| 5196 | 50 | 67 | 336 | hsa-miR-23a | 5298 | 6 | 2.6e−012 |
| 5197 | 50 | 66 | 306 | hsa-miR-23b | 18252 | 6 | 1.08e−012 |
| 5198 | 50 | 238 | 346, 416 | hsa-miR-24 | 20729 | 6 | 4.05e−011 |
| 5199 | 50 | 256 | 441, 487 | hsa-miR-26a | 16676 | 6 | 8.82e−011 |
| 5200 | 50 | 259 | 481 | hsa-miR-27a | 4572 | 6 | 2.31e−010 |
| 5201 | 50 | 260 | 280 | hsa-miR-27b | 4769 | 6 | 1.64e−011 |
| 5202 | 50 | 167 | 556 | hsa-miR-29a | 4702 | 6 | 1.36e−011 |
| 5203 | 50 | 168 | 303, 530 | hsa-miR-29b | 518 | 6 | 1.11e−010 |
| 5204 | 50 | 244 | 287 | hsa-miR-30a-5p | 864 | 6 | 1.43e−010 |
| 5205 | 50 | 242 | 311 | hsa-miR-30b | 1077 | 6 | 4.19e−011 |
| 5206 | 50 | 243 | 299, 316 | hsa-miR-30c | 2052 | 6 | 1.45e−011 |
| 5207 | 50 | 241 | 550 | hsa-miR-30d | 1846 | 6 | 2.78e−012 |
| 5208 | 50 | 245 | 431 | hsa-miR-30e-5p | 1257 | 6 | 1.25e−012 |
| 5209 | 50 | 1 | 359 | hsa-miR-320 | 2538 | 6 | 4.15e−012 |
| 5210 | 50 | 113 | 538 | hsa-miR-328 | 718 | 6 | 1.25e−012 |
| 5211 | 50 | 141 | 437 | hsa-miR-33 | 961 | 6 | 4.5e−013 |
| 5212 | 50 | 126 | 380 | hsa-miR-331 | 1204 | 6 | 8.84e−014 |
| 5213 | 50 | 194 | 337 | hsa-miR-338 | 3121 | 6 | 4.68e−012 |
| 5214 | 50 | 208 | 477 | hsa-miR-342 | 1512 | 6 | 1.68e−011 |
| 5215 | 50 | 237 | 480 | hsa-miR-34a | 955 | 6 | 8.7e−011 |
| 5216 | 50 | 112 | 372 | hsa-miR-422b | 528 | 6 | 2.75e−012 |
| 5217 | 50 | 51 | 497 | hsa-miR-423 | 963 | 6 | 6.56e−013 |
| 5218 | 50 | 214 | 376 | hsa-miR-494 | 3034 | 6 | 1.81e−009 |
| 5219 | 50 | 83 | 312 | hsa-miR-497 | 3799 | 6 | 2.26e−011 |
| 5220 | 50 | 135 | 315 | hsa-miR-565 | 963 | 6 | 1.37e−011 |
| 5221 | 50 | 60 | 479 | hsa-miR-638 | 2161 | 6 | 1.21e−010 |
| 5222 | 50 | 6 | 471 | hsa-miR-93 | 859 | 6 | 1.94e−010 |
| 5223 | 50 | 14 | 318 | hsa-miR-99a | 18260 | 6 | 1.49e−012 |
| 5224 | 50 | 80 | 509 | hsa-miR-99b | 1380 | 6 | 2.81e−013 |
| 5225 | 51 | 127 | 361 | 11_1 | 600 | 5 | 2.89e−009 |
| 5226 | 51 | 196 | 508 | 15_rel | 42404 | 5 | 1.28e−010 |
| 5227 | 51 | 94 | 394 | 69_6_rel | 1768 | 5 | 4.81e−008 |
| 5228 | 51 | 177 | 366, 464 | 72_12 | 2270 | 5 | 3.18e−008 |
| 5229 | 51 | 97 | 357 | 74_26_rel | 825 | 5 | 2.72e−009 |
| 5230 | 51 | 87 | 301 | gam11 | 14613 | 5 | 5.85e−009 |
| 5231 | 51 | 223 | 320, 393, 559 | hsa-let-7a | 5614 | 5 | 1.95e−009 |
| 5232 | 51 | 225 | 332 | hsa-let-7b | 13649 | 5 | 2.62e−008 |
| 5233 | 51 | 224 | 427 | hsa-let-7c | 7677 | 5 | 1.56e−009 |
| 5234 | 51 | 45 | 277 | hsa-let-7d | 2188 | 5 | 2.54e−009 |
| 5235 | 51 | 220 | 475 | hsa-let-7e | 1493 | 5 | 1.86e−009 |
| 5236 | 51 | 226 | 395 | hsa-let-7g | 599 | 5 | 1.6e−008 |
| 5237 | 51 | 227 | 297 | hsa-let-7i | 1119 | 5 | 3.19e−009 |
| 5238 | 51 | 13 | 397 | hsa-miR-100 | 512 | 5 | 7.67e−008 |
| 5239 | 51 | 48 | 483, 529 | hsa-miR-103 | 3112 | 5 | 2.21e−008 |
| 5240 | 51 | 184 | 423 | hsa-miR-105 | 853 | 5 | 3.84e−010 |
| 5241 | 51 | 2 | 399 | hsa-miR-106a | 1291 | 5 | 1.94e−008 |
| 5242 | 51 | 149 | 392 | hsa-miR-106b | 834 | 5 | 1.03e−008 |
| 5243 | 51 | 47 | 555 | hsa-miR-107 | 2645 | 5 | 6.09e−009 |
| 5244 | 51 | 199 | 387 | hsa-miR-125a | 2037 | 5 | 2.23e−009 |
| 5245 | 51 | 198 | 313, 551 | hsa-miR-125b | 4382 | 5 | 2.58e−007 |
| 5246 | 51 | 204 | 419 | hsa-miR-126 | 643 | 5 | 1.12e−007 |
| 5247 | 51 | 86 | 417 | hsa-miR-130a | 1260 | 5 | 4.02e−008 |
| 5248 | 51 | 151 | 472 | hsa-miR-141 | 3991 | 5 | 7.32e−008 |
| 5249 | 51 | 218 | 527 | hsa-miR-143 | 4026 | 5 | 1.52e−006 |
| 5250 | 51 | 139 | 281 | hsa-miR-145 | 2732 | 5 | 1.32e−006 |
| 5251 | 51 | 172 | 329 | hsa-miR-15b | 1010 | 5 | 1.94e−008 |
| 5252 | 51 | 173 | 360, 468 | hsa-miR-16 | 3373 | 5 | 3.54e−009 |
| 5253 | 51 | 75 | 467 | hsa-miR-17-5p | 756 | 5 | 1.96e−008 |
| 5254 | 51 | 10 | 371, 461 | hsa-miR-181a | 647 | 5 | 3.37e−008 |
| 5255 | 51 | 77 | 440 | hsa-miR-191 | 2524 | 5 | 2.29e−008 |
| 5256 | 51 | 17 | 442 | hsa-miR-193a | 1750 | 5 | 1.06e−008 |
| 5257 | 51 | 16 | 488 | hsa-miR-193b | 631 | 5 | 6.5e−010 |
| 5258 | 51 | 170 | 327 | hsa-miR-195 | 750 | 5 | 7.19e−009 |
| 5259 | 51 | 92 | 410, 432 | hsa-miR-199a | 1658 | 5 | 3.75e−007 |
| 5260 | 51 | 251 | 282, 495 | hsa-miR-19b | 739 | 5 | 1.95e−009 |
| 5261 | 51 | 152 | 369 | hsa-miR-200a | 4045 | 5 | 6.28e−009 |
| 5262 | 51 | 157 | 411 | hsa-miR-200b | 4019 | 5 | 3.56e−008 |
| 5263 | 51 | 156 | 446 | hsa-miR-200c | 7599 | 5 | 2.22e−008 |
| 5264 | 51 | 203 | 504 | hsa-miR-205 | 896 | 5 | 1.07e−007 |
| 5265 | 51 | 175 | 462 | hsa-miR-21 | 5995 | 5 | 1.78e−008 |
| 5266 | 51 | 115 | 314 | hsa-miR-210 | 532 | 5 | 1.9e−008 |
| 5267 | 51 | 29 | 439 | hsa-miR-214 | 521 | 5 | 3.54e−007 |
| 5268 | 51 | 18 | 322 | hsa-miR-22 | 1698 | 5 | 1.55e−008 |
| 5269 | 51 | 50 | 364 | hsa-miR-221 | 932 | 5 | 9.1e−008 |
| 5270 | 51 | 49 | 302 | hsa-miR-222 | 1241 | 5 | 1.03e−007 |
| 5271 | 51 | 67 | 336 | hsa-miR-23a | 3767 | 5 | 1.53e−009 |
| 5272 | 51 | 66 | 306 | hsa-miR-23b | 5479 | 5 | 6.41e−008 |
| 5273 | 51 | 238 | 346, 416 | hsa-miR-24 | 6530 | 5 | 2.17e−008 |
| 5274 | 51 | 90 | 388 | hsa-miR-25 | 500 | 5 | 6.34e−009 |
| 5275 | 51 | 256 | 441, 487 | hsa-miR-26a | 5150 | 5 | 1.01e−008 |
| 5276 | 51 | 259 | 481 | hsa-miR-27a | 3006 | 5 | 7.44e−010 |
| 5277 | 51 | 260 | 280 | hsa-miR-27b | 1083 | 5 | 7.72e−008 |
| 5278 | 51 | 61 | 286 | hsa-miR-296 | 546 | 5 | 1.08e−008 |
| 5279 | 51 | 167 | 556 | hsa-miR-29a | 1257 | 5 | 2.46e−008 |
| 5280 | 51 | 244 | 287 | hsa-miR-30a-5p | 811 | 5 | 1.54e−008 |
| 5281 | 51 | 242 | 311 | hsa-miR-30b | 771 | 5 | 2.29e−008 |
| 5282 | 51 | 243 | 299, 316 | hsa-miR-30c | 1346 | 5 | 4.71e−009 |
| 5283 | 51 | 241 | 550 | hsa-miR-30d | 1765 | 5 | 6.11e−009 |
| 5284 | 51 | 245 | 431 | hsa-miR-30e-5p | 1066 | 5 | 1.31e−008 |
| 5285 | 51 | 1 | 359 | hsa-miR-320 | 1343 | 5 | 4.09e−008 |
| 5286 | 51 | 113 | 538 | hsa-miR-328 | 521 | 5 | 6.81e−009 |
| 5287 | 51 | 141 | 437 | hsa-miR-33 | 823 | 5 | 3.96e−009 |
| 5288 | 51 | 126 | 380 | hsa-miR-331 | 931 | 5 | 2.25e−010 |
| 5289 | 51 | 194 | 337 | hsa-miR-338 | 1348 | 5 | 5.64e−013 |
| 5290 | 51 | 208 | 477 | hsa-miR-342 | 841 | 5 | 2.22e−008 |
| 5291 | 51 | 237 | 480 | hsa-miR-34a | 1232 | 5 | 5.04e−009 |
| 5292 | 51 | 176 | 403 | hsa-miR-34b | 796 | 5 | 1.16e−006 |
| 5293 | 51 | 57 | 498 | hsa-miR-34c | 1061 | 5 | 1.17e−006 |
| 5294 | 51 | 51 | 497 | hsa-miR-423 | 839 | 5 | 8.7e−009 |
| 5295 | 51 | 236 | 413 | hsa-miR-449 | 2709 | 5 | 3.78e−007 |
| 5296 | 51 | 58 | 465 | hsa-miR-449b | 1283 | 5 | 3.87e−006 |
| 5297 | 51 | 63 | 557 | hsa-miR-491 | 809 | 5 | 1.74e−009 |
| 5298 | 51 | 214 | 376 | hsa-miR-494 | 3368 | 5 | 1.05e−006 |
| 5299 | 51 | 83 | 312 | hsa-miR-497 | 1087 | 5 | 5.05e−009 |
| 5300 | 51 | 135 | 315 | hsa-miR-565 | 1706 | 5 | 1.24e−008 |
| 5301 | 51 | 53 | 459 | hsa-miR-613 | 528 | 5 | 1.01e−006 |
| 5302 | 51 | 60 | 479 | hsa-miR-638 | 1891 | 5 | 1.95e−008 |
| 5303 | 51 | 6 | 471 | hsa-miR-93 | 1548 | 5 | 4.01e−008 |
| 5304 | 51 | 14 | 318 | hsa-miR-99a | 2191 | 5 | 1.33e−007 |
| 5305 | 51 | 80 | 509 | hsa-miR-99b | 731 | 5 | 1.65e−009 |

TABLE 3

| RID | DID | MID | HIDs | mir name | Ratio | mean1 | mean2 | n1 | n2 | p_value |
|---|---|---|---|---|---|---|---|---|---|---|
| 1 | 1 | 223 | 320, 393, 559 | hsa-let-7a | 1.64 | 4057 | 6647 | 41 | 14 | 0.0178 |
| 2 | 1 | 225 | 332 | hsa-let-7b | 1.36 | 11159 | 8196 | 41 | 14 | 0.0162 |
| 3 | 1 | 224 | 427 | hsa-let-7c | 1.46 | 6062 | 8854 | 41 | 14 | 0.0125 |
| 4 | 1 | 222 | 482, 558 | hsa-let-7f | 1.54 | 492 | 756 | 41 | 14 | 0.0105 |
| 5 | 1 | 227 | 297 | hsa-let-7i | 1.28 | 1325 | 1692 | 41 | 14 | 0.0414 |
| 6 | 1 | 13 | 397 | hsa-miR-100 | 2.71 | 909 | 2462 | 41 | 14 | 0.00116 |
| 7 | 1 | 184 | 423 | hsa-miR-105 | 1.49 | 1138 | 765 | 41 | 14 | 0.00422 |
| 8 | 1 | 2 | 399 | hsa-miR-106a | 2.22 | 2153 | 972 | 41 | 14 | 7.6e−006 |
| 9 | 1 | 149 | 392 | hsa-miR-106b | 2.19 | 1738 | 794 | 41 | 14 | 2.36e−006 |
| 10 | 1 | 47 | 555 | hsa-miR-107 | 1.98 | 3083 | 1560 | 41 | 14 | 0.000612 |
| 11 | 1 | 199 | 387 | hsa-miR-125a | 2.1 | 1832 | 3844 | 41 | 14 | 5.02e−005 |
| 12 | 1 | 198 | 313, 551 | hsa-miR-125b | 3.51 | 3695 | 12970 | 41 | 14 | 0.000102 |
| 13 | 1 | 204 | 419 | hsa-miR-126 | 1.96 | 1684 | 3302 | 41 | 14 | 0.00452 |
| 14 | 1 | 85 | 328 | hsa-miR-130b | 2.96 | 502 | 170 | 41 | 14 | 2.76e−007 |
| 15 | 1 | 266 | 295, 386 | hsa-miR-133a | 4.17 | 432 | 1804 | 41 | 14 | 9.13e−011 |
| 16 | 1 | 64 | 402 | hsa-miR-140 | 1.96 | 514 | 262 | 41 | 14 | 0.00776 |
| 17 | 1 | 151 | 472 | hsa-miR-141 | 6.2 | 4820 | 777 | 41 | 14 | 4.17e−007 |
| 18 | 1 | 218 | 527 | hsa-miR-143 | 8.25 | 3340 | 27568 | 41 | 14 | 0.00137 |
| 19 | 1 | 139 | 281 | hsa-miR-145 | 3.55 | 7264 | 25760 | 41 | 14 | 0.000293 |
| 20 | 1 | 212 | 333 | hsa-miR-149 | 2.38 | 752 | 316 | 41 | 14 | 1.07e−006 |
| 21 | 1 | 34 | 396 | hsa-miR-151 | 2.66 | 691 | 259 | 41 | 14 | 1.25e−007 |
| 22 | 1 | 192 | 470 | hsa-miR-152 | 1.46 | 439 | 641 | 41 | 14 | 0.00141 |
| 23 | 1 | 38 | 467 | hsa-miR-17-3p | 1.68 | 551 | 328 | 41 | 14 | 0.00856 |
| 24 | 1 | 75 | 467 | hsa-miR-17-5p | 2.35 | 1647 | 701 | 41 | 14 | 1.57e−005 |
| 25 | 1 | 10 | 371, 461 | hsa-miR-181a | 1.85 | 1741 | 940 | 41 | 14 | 0.000428 |
| 26 | 1 | 11 | 456, 540 | hsa-miR-181b | 2.69 | 639 | 238 | 41 | 14 | 1.28e−007 |
| 27 | 1 | 179 | 356 | hsa-miR-183 | 3.42 | 610 | 179 | 41 | 14 | 0.000232 |
| 28 | 1 | 77 | 440 | hsa-miR-191 | 2.38 | 3020 | 1271 | 41 | 14 | 2.82e−005 |
| 29 | 1 | 17 | 442 | hsa-miR-193a | 2.26 | 1468 | 3324 | 41 | 14 | 0.000113 |
| 30 | 1 | 170 | 327 | hsa-miR-195 | 3.6 | 444 | 1598 | 41 | 14 | 3.04e−006 |
| 31 | 1 | 92 | 410, 432 | hsa-miR-199a | 1.81 | 2549 | 4607 | 41 | 14 | 0.022 |
| 32 | 1 | 162 | 410, 432, 473 | hsa-miR-199a* | 3.48 | 545 | 1899 | 41 | 14 | 6.65e−005 |
| 33 | 1 | 93 | 473 | hsa-miR-199b | 1.77 | 299 | 529 | 41 | 14 | 0.0021 |
| 34 | 1 | 157 | 411 | hsa-miR-200b | 3.38 | 2228 | 658 | 41 | 14 | 0.00233 |
| 35 | 1 | 156 | 446 | hsa-miR-200c | 5.29 | 5705 | 1078 | 41 | 14 | 1.2e−007 |
| 36 | 1 | 203 | 504 | hsa-miR-205 | 5.06 | 11272 | 2228 | 41 | 14 | 5.73e−006 |
| 37 | 1 | 115 | 314 | hsa-miR-210 | 4.2 | 1058 | 252 | 41 | 14 | 7.08e−010 |
| 38 | 1 | 29 | 439 | hsa-miR-214 | 1.71 | 1126 | 1925 | 41 | 14 | 0.0031 |
| 39 | 1 | 50 | 364 | hsa-miR-221 | 1.44 | 2262 | 3257 | 41 | 14 | 0.0387 |
| 40 | 1 | 49 | 302 | hsa-miR-222 | 1.6 | 2541 | 4071 | 41 | 14 | 0.00332 |
| 41 | 1 | 66 | 306 | hsa-miR-23b | 6.58 | 816 | 5369 | 41 | 14 | 0.015 |
| 42 | 1 | 260 | 280 | hsa-miR-27b | 2.49 | 1872 | 4670 | 41 | 14 | 4.66e−005 |
| 43 | 1 | 167 | 556 | hsa-miR-29a | 6.31 | 260 | 1639 | 41 | 14 | 0.000407 |
| 44 | 1 | 168 | 303, 530 | hsa-miR-29b | 3.9 | 141 | 551 | 41 | 14 | 0.000257 |
| 45 | 1 | 244 | 287 | hsa-miR-30a-5p | 4.55 | 260 | 1181 | 41 | 14 | 0.00146 |
| 46 | 1 | 242 | 311 | hsa-miR-30b | 1.61 | 544 | 875 | 41 | 14 | 0.0334 |
| 47 | 1 | 241 | 550 | hsa-miR-30d | 1.62 | 1645 | 1019 | 41 | 14 | 0.0322 |
| 48 | 1 | 132 | 429 | hsa-miR-31 | 2.44 | 2069 | 847 | 41 | 14 | 0.00543 |
| 49 | 1 | 1 | 359 | hsa-miR-320 | 1.63 | 4377 | 2692 | 41 | 14 | 0.000571 |
| 50 | 1 | 98 | 368 | hsa-miR-324-5p | 1.39 | 542 | 390 | 41 | 14 | 0.041 |
| 51 | 1 | 229 | 292 | hsa-miR-345 | 2.4 | 638 | 266 | 41 | 14 | 6.05e−005 |
| 52 | 1 | 237 | 480 | hsa-miR-34a | 1.63 | 2690 | 1648 | 41 | 14 | 0.00144 |
| 53 | 1 | 128 | 294 | hsa-miR-370 | 1.76 | 678 | 386 | 41 | 14 | 0.0122 |
| 54 | 1 | 51 | 497 | hsa-miR-423 | 1.4 | 2172 | 1553 | 41 | 14 | 0.00996 |
| 55 | 1 | 236 | 413 | hsa-miR-449 | 2.79 | 1380 | 495 | 41 | 14 | 0.00441 |
| 56 | 1 | 252 | 405 | hsa-miR-452 | 3.51 | 518 | 147 | 41 | 14 | 1.34e−005 |
| 57 | 1 | 63 | 557 | hsa-miR-491 | 1.95 | 2162 | 1110 | 41 | 14 | 0.0211 |
| 58 | 1 | 54 | 293 | hsa-miR-492 | 2.58 | 543 | 210 | 41 | 14 | 0.000276 |
| 59 | 1 | 83 | 312 | hsa-miR-497 | 2.62 | 840 | 2197 | 41 | 14 | 6.79e−006 |
| 60 | 1 | 258 | 428, 443 | hsa-miR-513 | 2.16 | 609 | 282 | 41 | 14 | 0.00733 |
| 61 | 1 | 210 | 400 | hsa-miR-518c* | 2.71 | 506 | 187 | 41 | 14 | 0.000365 |
| 62 | 1 | 182 | 304, 317 | hsa-miR-92 | 1.75 | 2708 | 1546 | 41 | 14 | 0.00096 |
| 63 | 1 | 6 | 471 | hsa-miR-93 | 2.9 | 2783 | 959 | 41 | 14 | 8.57e−008 |
| 64 | 1 | 14 | 318 | hsa-miR-99a | 2.78 | 1288 | 3584 | 41 | 14 | 0.00162 |
| 65 | 2 | 196 | 508 | 15_rel | 3.9 | 31522 | 8090 | 19 | 10 | 0.0408 |
| 66 | 2 | 94 | 394 | 69_6_rel | 3.38 | 6919 | 2047 | 19 | 10 | 0.024 |
| 67 | 2 | 121 | 502 | 70_14 | 2.04 | 248 | 506 | 19 | 10 | 0.00799 |
| 68 | 2 | 40 | 553 | 72_4 | 2.59 | 632 | 1634 | 19 | 10 | 0.0163 |
| 69 | 2 | 87 | 301 | gam11 | 7.8 | 1703 | 219 | 19 | 10 | 0.0418 |
| 70 | 2 | 163 | 402 | 43_7 | 1.79 | 374 | 672 | 19 | 10 | 0.0339 |
| 71 | 2 | 222 | 482, 558 | hsa-let-7f | 1.44 | 420 | 604 | 21 | 12 | 0.0466 |
| 72 | 2 | 13 | 397 | hsa-miR-100 | 2.91 | 1172 | 402 | 21 | 12 | 0.00391 |
| 73 | 2 | 198 | 313, 551 | hsa-miR-125b | 3.65 | 4768 | 1306 | 21 | 12 | 5.31e−005 |
| 74 | 2 | 89 | 419 | hsa-miR-126* | 2.31 | 565 | 244 | 21 | 12 | 0.00124 |
| 75 | 2 | 151 | 472 | hsa-miR-141 | 2.44 | 2374 | 5798 | 21 | 12 | 0.0068 |
| 76 | 2 | 139 | 281 | hsa-miR-145 | 4.28 | 8353 | 1954 | 21 | 12 | 3.07e−005 |
| 77 | 2 | 191 | 289 | hsa-miR-148b | 1.26 | 398 | 503 | 21 | 12 | 0.0384 |
| 78 | 2 | 77 | 440 | hsa-miR-191 | 1.98 | 1727 | 3426 | 21 | 12 | 0.000489 |
| 79 | 2 | 16 | 488 | hsa-miR-193b | 3.79 | 661 | 174 | 21 | 12 | 0.0272 |
| 80 | 2 | 92 | 410, 432 | hsa-miR-199a | 2.26 | 2846 | 1259 | 21 | 12 | 0.0353 |
| 81 | 2 | 156 | 446 | hsa-miR-200c | 2.1 | 3026 | 6368 | 21 | 12 | 0.0187 |
| 82 | 2 | 175 | 462 | hsa-miR-21 | 2.16 | 6398 | 2957 | 21 | 12 | 0.000678 |
| 83 | 2 | 115 | 314 | hsa-miR-210 | 1.56 | 751 | 1174 | 21 | 12 | 0.00405 |
| 84 | 2 | 18 | 322 | hsa-miR-22 | 1.6 | 2919 | 1823 | 21 | 12 | 0.0175 |
| 85 | 2 | 50 | 364 | hsa-miR-221 | 1.78 | 2152 | 1206 | 21 | 12 | 0.0067 |
| 86 | 2 | 49 | 302 | hsa-miR-222 | 1.54 | 2351 | 1530 | 21 | 12 | 0.0169 |
| 87 | 2 | 111 | 389 | hsa-miR-422a | 1.43 | 412 | 587 | 21 | 12 | 0.00768 |
| 88 | 2 | 158 | 378 | hsa-miR-429 | 1.57 | 335 | 525 | 21 | 12 | 0.0335 |
| 89 | 2 | 206 | 513 | hsa-miR-520d* | 2.02 | 529 | 261 | 21 | 12 | 0.0228 |
| 90 | 2 | 14 | 318 | hsa-miR-99a | 2.84 | 1642 | 578 | 21 | 12 | 0.00384 |
| 91 | 3 | 127 | 361 | 11_1 | 15.6 | 1472 | 22971 | 21 | 5 | 3.41e−005 |
| 92 | 3 | 196 | 508 | 15_rel | 5.45 | 119454 | 21905 | 19 | 5 | 0.000157 |
| 93 | 3 | 219 | 497 | 27_3 | 1.97 | 5031 | 2560 | 21 | 5 | 0.0113 |
| 94 | 3 | 122 | 281 | 43_11 | 3.11 | 507 | 163 | 21 | 5 | 0.0104 |
| 95 | 3 | 42 | 449 | 43_3 | 4.04 | 536 | 133 | 21 | 5 | 0.00286 |
| 96 | 3 | 101 | 475 | 43_30 | 2.97 | 753 | 254 | 21 | 5 | 0.000187 |
| 97 | 3 | 129 | 548 | 43_39 | 4.01 | 1874 | 467 | 21 | 5 | 0.00342 |
| 98 | 3 | 27 | 313 | 43_40 | 2.21 | 691 | 313 | 21 | 5 | 0.00508 |
| 99 | 3 | 143 | 416 | 43_43 | 7.95 | 72 | 574 | 19 | 5 | 3e−005 |
| 100 | 3 | 183 | 373 | 66_1 | 6.63 | 1287 | 194 | 21 | 5 | 8.93e−005 |
| 101 | 3 | 94 | 394 | 69_6_rel | 4.48 | 33718 | 7530 | 19 | 5 | 0.00427 |
| 102 | 3 | 177 | 366, 464 | 72_12 | 4.5 | 3531 | 15894 | 19 | 5 | 0.00287 |
| 103 | 3 | 40 | 553 | 72_4 | 8.74 | 1487 | 12994 | 19 | 5 | 0.00247 |
| 104 | 3 | 97 | 357 | 74_26_rel | 3.38 | 7104 | 2101 | 19 | 5 | 0.0212 |
| 105 | 3 | 223 | 320, 393, 559 | hsa-let-7a | 2.48 | 10243 | 25447 | 21 | 5 | 0.0157 |
| 106 | 3 | 45 | 277 | hsa-let-7d | 1.97 | 6042 | 11912 | 21 | 5 | 0.017 |
| 107 | 3 | 48 | 483, 529 | hsa-miR-103 | 3.06 | 8136 | 24861 | 21 | 5 | 0.0131 |
| 108 | 3 | 184 | 423 | hsa-miR-105 | 1.83 | 2833 | 5189 | 21 | 5 | 0.00246 |
| 109 | 3 | 2 | 399 | hsa-miR-106a | 1.99 | 5589 | 11138 | 21 | 5 | 0.0492 |
| 110 | 3 | 149 | 392 | hsa-miR-106b | 2.59 | 4530 | 11756 | 21 | 5 | 4.72e−006 |
| 111 | 3 | 47 | 555 | hsa-miR-107 | 3.02 | 7141 | 21548 | 21 | 5 | 0.00109 |
| 112 | 3 | 166 | 476 | hsa-miR-10a | 7.04 | 153 | 1078 | 21 | 5 | 2.45e−005 |
| 113 | 3 | 165 | 340 | hsa-miR-10b | 4.66 | 177 | 824 | 21 | 5 | 0.00116 |
| 114 | 3 | 198 | 313, 551 | hsa-miR-125b | 4.66 | 21312 | 4575 | 21 | 5 | 0.000502 |
| 115 | 3 | 204 | 419 | hsa-miR-126 | 2.89 | 4083 | 11784 | 21 | 5 | 0.0162 |
| 116 | 3 | 89 | 419 | hsa-miR-126* | 4.3 | 1419 | 330 | 21 | 5 | 0.00045 |
| 117 | 3 | 188 | 491 | hsa-miR-128a | 3.14 | 291 | 913 | 21 | 5 | 0.00218 |
| 118 | 3 | 187 | 383 | hsa-miR-128b | 11.44 | 72 | 820 | 19 | 5 | 2.75e−006 |
| 119 | 3 | 86 | 417 | hsa-miR-130a | 1.99 | 6227 | 3130 | 21 | 5 | 0.0203 |
| 120 | 3 | 266 | 295, 386 | hsa-miR-133a | 2.89 | 1109 | 383 | 21 | 5 | 0.0051 |
| 121 | 3 | 265 | 533 | hsa-miR-133b | 2.12 | 669 | 315 | 19 | 5 | 0.048 |
| 122 | 3 | 52 | 370, 548 | hsa-miR-138 | 2.53 | 1228 | 486 | 21 | 5 | 0.00116 |
| 123 | 3 | 218 | 527 | hsa-miR-143 | 74.33 | 17437 | 235 | 21 | 5 | 0.0024 |
| 124 | 3 | 139 | 281 | hsa-miR-145 | 2.65 | 36737 | 13879 | 21 | 5 | 0.0202 |
| 125 | 3 | 146 | 325 | hsa-miR-147 | 4.73 | 529 | 112 | 21 | 5 | 0.000797 |
| 126 | 3 | 190 | 458 | hsa-miR-148a | 2.1 | 468 | 985 | 21 | 5 | 0.0228 |
| 127 | 3 | 191 | 289 | hsa-miR-148b | 2.87 | 853 | 2450 | 21 | 5 | 1.69e−005 |
| 128 | 3 | 34 | 396 | hsa-miR-151 | 2.21 | 1388 | 3072 | 21 | 5 | 0.00058 |
| 129 | 3 | 171 | 331 | hsa-miR-15a | 3.09 | 719 | 2218 | 21 | 5 | 4.62e−005 |
| 130 | 3 | 172 | 329 | hsa-miR-15b | 3.81 | 1828 | 6961 | 21 | 5 | 0.000303 |
| 131 | 3 | 173 | 360, 468 | hsa-miR-16 | 3.02 | 9271 | 27955 | 21 | 5 | 0.00703 |
| 132 | 3 | 38 | 467 | hsa-miR-17-3p | 2.18 | 1308 | 2850 | 21 | 5 | 0.000348 |
| 133 | 3 | 75 | 467 | hsa-miR-17-5p | 2.39 | 4217 | 10094 | 21 | 5 | 0.0111 |
| 134 | 3 | 9 | 451 | hsa-miR-181c | 3.15 | 522 | 1641 | 21 | 5 | 0.0014 |
| 135 | 3 | 272 | 323 | hsa-miR-182 | 5.51 | 682 | 3758 | 21 | 5 | 0.000863 |
| 136 | 3 | 233 | 305 | hsa-miR-184 | 2.9 | 562 | 194 | 21 | 5 | 0.00829 |
| 137 | 3 | 144 | 346 | hsa-miR-189 | 1.94 | 690 | 356 | 21 | 5 | 0.0199 |
| 138 | 3 | 155 | 426 | hsa-miR-18a | 2.31 | 927 | 2145 | 21 | 5 | 0.00394 |
| 139 | 3 | 77 | 440 | hsa-miR-191 | 3.39 | 6063 | 20537 | 21 | 5 | 0.000653 |
| 140 | 3 | 130 | 440 | hsa-miR-191* | 3.5 | 1021 | 292 | 19 | 5 | 0.00895 |
| 141 | 3 | 246 | 290, 549 | hsa-miR-194 | 2.88 | 279 | 806 | 21 | 5 | 0.00456 |
| 142 | 3 | 136 | 308 | hsa-miR-198 | 2.67 | 629 | 235 | 21 | 5 | 0.0203 |
| 143 | 3 | 251 | 282, 495 | hsa-miR-19b | 3.63 | 3029 | 10997 | 21 | 5 | 0.00144 |
| 144 | 3 | 157 | 411 | hsa-miR-200b | 5.38 | 4682 | 25168 | 21 | 5 | 0.0087 |
| 145 | 3 | 271 | 334 | hsa-miR-202* | 2.71 | 700 | 259 | 21 | 5 | 0.027 |
| 146 | 3 | 140 | 420 | hsa-miR-203 | 6.14 | 511 | 3136 | 21 | 5 | 0.0108 |
| 147 | 3 | 232 | 422 | hsa-miR-206 | 4.19 | 584 | 139 | 21 | 5 | 0.00661 |
| 148 | 3 | 150 | 351 | hsa-miR-20a | 3.42 | 561 | 1920 | 21 | 5 | 0.00147 |
| 149 | 3 | 115 | 314 | hsa-miR-210 | 3.39 | 2072 | 7034 | 21 | 5 | 0.000717 |
| 150 | 3 | 29 | 439 | hsa-miR-214 | 3.59 | 3988 | 1110 | 21 | 5 | 0.00116 |
| 151 | 3 | 159 | 494 | hsa-miR-216 | 4.47 | 1267 | 284 | 21 | 5 | 5.41e−005 |
| 152 | 3 | 248 | 415 | hsa-miR-223 | 1.91 | 720 | 377 | 21 | 5 | 0.0405 |
| 153 | 3 | 67 | 336 | hsa-miR-23a | 2.37 | 11188 | 26475 | 21 | 5 | 0.0399 |
| 154 | 3 | 19 | 457 | hsa-miR-28 | 2.22 | 339 | 754 | 21 | 5 | 0.0443 |
| 155 | 3 | 61 | 286 | hsa-miR-296 | 24.81 | 3113 | 125 | 21 | 5 | 0.000139 |
| 156 | 3 | 167 | 556 | hsa-miR-29a | 11.28 | 767 | 68 | 21 | 5 | 0.0159 |
| 157 | 3 | 169 | 528 | hsa-miR-29c | 5.86 | 264 | 1544 | 21 | 5 | 0.000179 |
| 158 | 3 | 84 | 489 | hsa-miR-301 | 2.66 | 189 | 502 | 21 | 5 | 0.0463 |
| 159 | 3 | 269 | 362 | hsa-miR-302c* | 3.18 | 587 | 185 | 21 | 5 | 0.00181 |
| 160 | 3 | 117 | 287 | hsa-miR-30a-3p | 2.48 | 1038 | 419 | 21 | 5 | 0.000136 |
| 161 | 3 | 242 | 311 | hsa-miR-30b | 4.25 | 943 | 4009 | 21 | 5 | 0.000763 |
| 162 | 3 | 243 | 299, 316 | hsa-miR-30c | 7.54 | 1962 | 14783 | 21 | 5 | 0.00126 |
| 163 | 3 | 241 | 550 | hsa-miR-30d | 2.7 | 4126 | 11138 | 21 | 5 | 0.000172 |
| 164 | 3 | 245 | 431 | hsa-miR-30e-5p | 3.05 | 4433 | 13506 | 21 | 5 | 0.00059 |
| 165 | 3 | 132 | 429 | hsa-miR-31 | 3.21 | 4132 | 13261 | 21 | 5 | 0.0485 |
| 166 | 3 | 1 | 359 | hsa-miR-320 | 2.12 | 15982 | 7552 | 21 | 5 | 0.00316 |
| 167 | 3 | 98 | 368 | hsa-miR-324-5p | 1.57 | 1426 | 909 | 21 | 5 | 0.0152 |
| 168 | 3 | 113 | 538 | hsa-miR-328 | 4.63 | 1669 | 360 | 21 | 5 | 0.000911 |
| 169 | 3 | 185 | 492 | hsa-miR-335 | 7.15 | 1062 | 149 | 21 | 5 | 0.00246 |
| 170 | 3 | 194 | 337 | hsa-miR-338 | 13.27 | 1640 | 124 | 21 | 5 | 0.00398 |
| 171 | 3 | 200 | 448 | hsa-miR-339 | 3.14 | 551 | 1729 | 21 | 5 | 0.0019 |
| 172 | 3 | 237 | 480 | hsa-miR-34a | 2.36 | 6918 | 16339 | 21 | 5 | 0.000494 |
| 173 | 3 | 57 | 498 | hsa-miR-34c | 2.04 | 450 | 918 | 21 | 5 | 0.0409 |
| 174 | 3 | 128 | 294 | hsa-miR-370 | 3.48 | 2237 | 643 | 21 | 5 | 0.00144 |
| 175 | 3 | 254 | 496 | hsa-miR-374 | 4.82 | 588 | 122 | 21 | 5 | 0.000532 |
| 176 | 3 | 51 | 497 | hsa-miR-423 | 1.66 | 7077 | 4251 | 21 | 5 | 0.0287 |
| 177 | 3 | 158 | 378 | hsa-miR-429 | 4.83 | 631 | 3047 | 21 | 5 | 0.00231 |
| 178 | 3 | 213 | 531 | hsa-miR-432 | 5.48 | 622 | 114 | 21 | 5 | 0.000236 |
| 179 | 3 | 236 | 413 | hsa-miR-449 | 8.24 | 5159 | 626 | 21 | 5 | 0.00166 |
| 180 | 3 | 4 | 430 | hsa-miR-451 | 10.42 | 197 | 2048 | 19 | 5 | 0.00174 |
| 181 | 3 | 189 | 500 | hsa-miR-484 | 3.73 | 371 | 1387 | 19 | 5 | 0.00276 |
| 182 | 3 | 63 | 557 | hsa-miR-491 | 6.56 | 9802 | 1495 | 21 | 5 | 0.000574 |
| 183 | 3 | 267 | 354 | hsa-miR-493-5p | 6.53 | 755 | 116 | 21 | 5 | 0.000222 |
| 184 | 3 | 214 | 376 | hsa-miR-494 | 3.18 | 16273 | 5117 | 21 | 5 | 0.0312 |
| 185 | 3 | 3 | 543 | hsa-miR-495 | 3.44 | 744 | 216 | 21 | 5 | 0.0164 |
| 186 | 3 | 68 | 288 | hsa-miR-502 | 3.46 | 552 | 160 | 21 | 5 | 0.00213 |
| 187 | 3 | 43 | 418 | hsa-miR-504 | 2.14 | 1855 | 867 | 21 | 5 | 0.0353 |
| 188 | 3 | 73 | 503 | hsa-miR-518b | 4.33 | 938 | 217 | 21 | 5 | 0.00185 |
| 189 | 3 | 262 | 523 | hsa-miR-519e* | 3.37 | 727 | 215 | 21 | 5 | 0.0131 |
| 190 | 3 | 102 | 344 | hsa-miR-520a* | 3.99 | 974 | 244 | 21 | 5 | 0.000605 |
| 191 | 3 | 206 | 513 | hsa-miR-520d* | 3.45 | 1268 | 367 | 21 | 5 | 0.013 |
| 192 | 3 | 100 | 524 | hsa-miR-524* | 3.45 | 883 | 256 | 21 | 5 | 0.0104 |
| 193 | 3 | 110 | 511, 512, 514 | hsa-miR-527 | 3.87 | 1064 | 275 | 21 | 5 | 0.00895 |
| 194 | 3 | 8 | 276 | hsa-miR-552 | 8.13 | 71 | 575 | 19 | 5 | 7.78e−006 |
| 195 | 3 | 60 | 479 | hsa-miR-638 | 5.09 | 40272 | 7919 | 19 | 5 | 0.000967 |
| 196 | 3 | 164 | 381 | hsa-miR-660 | 9.45 | 68 | 647 | 19 | 5 | 1.31e−006 |
| 197 | 3 | 6 | 471 | hsa-miR-93 | 1.95 | 7735 | 15103 | 21 | 5 | 0.0307 |
| 198 | 3 | 215 | 528 | 43_5 | 19.44 | 76 | 1470 | 19 | 5 | 2.49e−008 |
| 199 | 4 | 127 | 361 | 11_1 | 10.29 | 23102 | 2245 | 5 | 12 | 0.000379 |
| 200 | 4 | 42 | 449 | 43_3 | 4.58 | 134 | 616 | 5 | 12 | 0.00876 |
| 201 | 4 | 99 | 348, 384, 434 | 43_35 | 2.82 | 199 | 562 | 5 | 12 | 0.0295 |
| 202 | 4 | 129 | 548 | 43_39 | 3.49 | 456 | 1590 | 5 | 12 | 0.0174 |
| 203 | 4 | 143 | 416 | 43_43 | 6.54 | 561 | 86 | 5 | 10 | 0.00229 |
| 204 | 4 | 183 | 373 | 66_1 | 3.72 | 195 | 727 | 5 | 12 | 0.0229 |
| 205 | 4 | 177 | 366, 464 | 72_12 | 4.65 | 15793 | 3400 | 5 | 10 | 0.00161 |
| 206 | 4 | 97 | 357 | 74_26_rel | 4.71 | 2026 | 9533 | 5 | 10 | 0.00262 |
| 207 | 4 | 87 | 301 | gam11 | 32.2 | 10448 | 324 | 5 | 10 | 0.0294 |
| 208 | 4 | 223 | 320, 393, 559 | hsa-let-7a | 1.75 | 25590 | 14603 | 5 | 12 | 0.0357 |
| 209 | 4 | 224 | 427 | hsa-let-7c | 1.64 | 28970 | 17713 | 5 | 12 | 0.0391 |
| 210 | 4 | 226 | 395 | hsa-let-7g | 1.92 | 2080 | 4004 | 5 | 12 | 0.0175 |
| 211 | 4 | 231 | 321, 424 | hsa-miR-1 | 4.21 | 124 | 521 | 5 | 12 | 0.00419 |
| 212 | 4 | 149 | 392 | hsa-miR-106b | 2.35 | 11694 | 4984 | 5 | 12 | 0.000261 |
| 213 | 4 | 166 | 476 | hsa-miR-10a | 2.97 | 1048 | 353 | 5 | 12 | 0.0242 |
| 214 | 4 | 165 | 340 | hsa-miR-10b | 2.6 | 800 | 308 | 5 | 12 | 0.0371 |
| 215 | 4 | 188 | 491 | hsa-miR-128a | 2.1 | 885 | 421 | 5 | 12 | 0.00709 |
| 216 | 4 | 187 | 383 | hsa-miR-128b | 11.44 | 796 | 70 | 5 | 10 | 1.22e−005 |
| 217 | 4 | 86 | 417 | hsa-miR-130a | 1.94 | 3046 | 5897 | 5 | 12 | 0.00459 |
| 218 | 4 | 37 | 391 | hsa-miR-136 | 6.75 | 695 | 103 | 5 | 12 | 0.00379 |
| 219 | 4 | 218 | 527 | hsa-miR-143 | 22 | 239 | 5253 | 5 | 12 | 0.0346 |
| 220 | 4 | 146 | 325 | hsa-miR-147 | 4.87 | 114 | 554 | 5 | 12 | 0.00648 |
| 221 | 4 | 191 | 289 | hsa-miR-148b | 2.08 | 2384 | 1147 | 5 | 12 | 0.00317 |
| 222 | 4 | 172 | 329 | hsa-miR-15b | 5.23 | 6862 | 1312 | 5 | 12 | 0.0414 |
| 223 | 4 | 173 | 360, 468 | hsa-miR-16 | 2.15 | 28145 | 13110 | 5 | 12 | 0.00562 |
| 224 | 4 | 75 | 467 | hsa-miR-17-5p | 2.19 | 10048 | 4587 | 5 | 12 | 0.00943 |
| 225 | 4 | 9 | 451 | hsa-miR-181c | 2.84 | 1596 | 563 | 5 | 12 | 0.0146 |
| 226 | 4 | 272 | 323 | hsa-miR-182 | 3.59 | 3674 | 1024 | 5 | 12 | 0.0288 |
| 227 | 4 | 233 | 305 | hsa-miR-184 | 3 | 195 | 585 | 5 | 12 | 0.0367 |
| 228 | 4 | 144 | 346 | hsa-miR-189 | 2.18 | 348 | 758 | 5 | 12 | 0.0246 |
| 229 | 4 | 155 | 426 | hsa-miR-18a | 2.87 | 2084 | 726 | 5 | 12 | 0.00546 |
| 230 | 4 | 16 | 488 | hsa-miR-193b | 13.69 | 3821 | 279 | 5 | 12 | 0.0197 |
| 231 | 4 | 246 | 290, 549 | hsa-miR-194 | 2.1 | 782 | 373 | 5 | 12 | 0.0192 |
| 232 | 4 | 251 | 282, 495 | hsa-miR-19b | 6.67 | 10960 | 1643 | 5 | 12 | 0.0289 |
| 233 | 4 | 271 | 334 | hsa-miR-202* | 2.78 | 257 | 716 | 5 | 12 | 0.0489 |
| 234 | 4 | 203 | 504 | hsa-miR-205 | 1.85 | 33500 | 61945 | 5 | 12 | 0.0497 |
| 235 | 4 | 232 | 422 | hsa-miR-206 | 4.72 | 141 | 666 | 5 | 12 | 0.0102 |
| 236 | 4 | 150 | 351 | hsa-miR-20a | 4.09 | 1865 | 457 | 5 | 12 | 2.61e−006 |
| 237 | 4 | 29 | 439 | hsa-miR-214 | 2.78 | 1072 | 2978 | 5 | 12 | 0.00776 |
| 238 | 4 | 268 | 401, 425 | hsa-miR-218 | 3.18 | 194 | 616 | 5 | 12 | 0.0281 |
| 239 | 4 | 18 | 322 | hsa-miR-22 | 2.48 | 16859 | 6790 | 5 | 12 | 0.0334 |
| 240 | 4 | 67 | 336 | hsa-miR-23a | 2.49 | 26563 | 10661 | 5 | 12 | 0.0406 |
| 241 | 4 | 90 | 388 | hsa-miR-25 | 13.88 | 3175 | 229 | 5 | 12 | 0.00842 |
| 242 | 4 | 256 | 441, 487 | hsa-miR-26a | 1.46 | 30109 | 20624 | 5 | 12 | 0.0402 |
| 243 | 4 | 259 | 481 | hsa-miR-27a | 1.86 | 26114 | 14027 | 5 | 12 | 0.037 |
| 244 | 4 | 19 | 457 | hsa-miR-28 | 1.91 | 733 | 384 | 5 | 12 | 0.0245 |
| 245 | 4 | 61 | 286 | hsa-miR-296 | 10.49 | 127 | 1333 | 5 | 12 | 0.0298 |
| 246 | 4 | 169 | 528 | hsa-miR-29c | 2.62 | 1496 | 571 | 5 | 12 | 0.0116 |
| 247 | 4 | 269 | 362 | hsa-miR-302c* | 2.77 | 186 | 515 | 5 | 12 | 0.0443 |
| 248 | 4 | 242 | 311 | hsa-miR-30b | 3.34 | 3912 | 1173 | 5 | 12 | 0.0157 |
| 249 | 4 | 243 | 299, 316 | hsa-miR-30c | 6.79 | 14770 | 2176 | 5 | 12 | 0.0472 |
| 250 | 4 | 241 | 550 | hsa-miR-30d | 2.49 | 11086 | 4458 | 5 | 12 | 0.0126 |
| 251 | 4 | 245 | 431 | hsa-miR-30e-5p | 2.53 | 13499 | 5326 | 5 | 12 | 0.00687 |
| 252 | 4 | 1 | 359 | hsa-miR-320 | 2.02 | 7395 | 14976 | 5 | 12 | 0.000537 |
| 253 | 4 | 113 | 538 | hsa-miR-328 | 4.76 | 359 | 1706 | 5 | 12 | 0.0186 |
| 254 | 4 | 185 | 492 | hsa-miR-335 | 5.53 | 150 | 830 | 5 | 12 | 0.0224 |
| 255 | 4 | 194 | 337 | hsa-miR-338 | 5.21 | 126 | 653 | 5 | 12 | 0.039 |
| 256 | 4 | 200 | 448 | hsa-miR-339 | 2.63 | 1674 | 635 | 5 | 12 | 1.35e−005 |
| 257 | 4 | 237 | 480 | hsa-miR-34a | 1.81 | 16279 | 9010 | 5 | 12 | 0.028 |
| 258 | 4 | 255 | 444 | hsa-miR-361 | 10.5 | 2081 | 198 | 5 | 12 | 0.00998 |
| 259 | 4 | 254 | 496 | hsa-miR-374 | 4.34 | 124 | 537 | 5 | 12 | 0.00623 |
| 260 | 4 | 46 | 298 | hsa-miR-383 | 3.84 | 140 | 538 | 5 | 12 | 0.0101 |
| 261 | 4 | 51 | 497 | hsa-miR-423 | 2.07 | 4139 | 8565 | 5 | 12 | 0.00244 |
| 262 | 4 | 158 | 378 | hsa-miR-429 | 2.43 | 2978 | 1227 | 5 | 12 | 0.000412 |
| 263 | 4 | 213 | 531 | hsa-miR-432 | 4.67 | 115 | 539 | 5 | 12 | 0.00498 |
| 264 | 4 | 236 | 413 | hsa-miR-449 | 8.24 | 620 | 5108 | 5 | 12 | 0.0106 |
| 265 | 4 | 274 | 375, 309 | hsa-miR-450 | 5.17 | 101 | 523 | 5 | 12 | 0.00317 |
| 266 | 4 | 189 | 500 | hsa-miR-484 | 3.25 | 1342 | 412 | 5 | 10 | 0.00103 |
| 267 | 4 | 63 | 557 | hsa-miR-491 | 5.08 | 1442 | 7330 | 5 | 12 | 0.00173 |
| 268 | 4 | 267 | 354 | hsa-miR-493-5p | 6.15 | 118 | 722 | 5 | 12 | 0.00381 |
| 269 | 4 | 214 | 376 | hsa-miR-494 | 6.32 | 4946 | 31235 | 5 | 12 | 0.00399 |
| 270 | 4 | 3 | 543 | hsa-miR-495 | 3.81 | 217 | 828 | 5 | 12 | 0.0383 |
| 271 | 4 | 68 | 288 | hsa-miR-502 | 3.42 | 161 | 550 | 5 | 12 | 0.0117 |
| 272 | 4 | 8 | 276 | hsa-miR-552 | 7.2 | 561 | 78 | 5 | 10 | 0.000635 |
| 273 | 4 | 60 | 479 | hsa-miR-638 | 5.86 | 7763 | 45456 | 5 | 10 | 6.74e−005 |
| 274 | 4 | 164 | 381 | hsa-miR-660 | 9.57 | 629 | 66 | 5 | 10 | 2.72e−005 |
| 275 | 4 | 230 | 474, 484, 536 | hsa-miR-7 | 5.92 | 103 | 610 | 5 | 10 | 0.0113 |
| 276 | 4 | 215 | 528 | 43_5 | 15.06 | 1431 | 95 | 5 | 10 | 0.000123 |
| 277 | 5 | 13 | 397 | hsa-miR-100 | 1.9 | 1187 | 2251 | 15 | 6 | 0.0402 |
| 278 | 5 | 149 | 392 | hsa-miR-106b | 1.58 | 1532 | 967 | 15 | 6 | 0.0308 |
| 279 | 5 | 198 | 313, 551 | hsa-miR-125b | 2.17 | 6368 | 13790 | 15 | 6 | 0.0205 |
| 280 | 5 | 204 | 419 | hsa-miR-126 | 2.11 | 2277 | 4802 | 15 | 6 | 0.0194 |
| 281 | 5 | 151 | 472 | hsa-miR-141 | 2.38 | 3701 | 1553 | 15 | 6 | 0.00288 |
| 282 | 5 | 218 | 527 | hsa-miR-143 | 2.1 | 4012 | 8416 | 15 | 6 | 0.0177 |
| 283 | 5 | 139 | 281 | hsa-miR-145 | 2.23 | 3139 | 7002 | 15 | 6 | 0.03 |
| 284 | 5 | 191 | 289 | hsa-miR-148b | 1.71 | 657 | 384 | 15 | 6 | 0.00408 |
| 285 | 5 | 272 | 323 | hsa-miR-182 | 2.35 | 572 | 244 | 15 | 6 | 0.0283 |
| 286 | 5 | 179 | 356 | hsa-miR-183 | 3.07 | 1623 | 528 | 15 | 6 | 0.0494 |
| 287 | 5 | 170 | 327 | hsa-miR-195 | 2.09 | 1312 | 2741 | 15 | 6 | 0.0255 |
| 288 | 5 | 93 | 473 | hsa-miR-199b | 2.47 | 622 | 1539 | 15 | 6 | 0.0188 |
| 289 | 5 | 156 | 446 | hsa-miR-200c | 1.74 | 4605 | 2653 | 15 | 6 | 0.0405 |
| 290 | 5 | 115 | 314 | hsa-miR-210 | 2.1 | 586 | 280 | 15 | 6 | 0.0426 |
| 291 | 5 | 83 | 312 | hsa-miR-497 | 1.84 | 1629 | 2996 | 15 | 6 | 0.018 |
| 292 | 5 | 14 | 318 | hsa-miR-99a | 2.06 | 2975 | 6124 | 15 | 6 | 0.0247 |
| 293 | 6 | 161 | 438, 561 | hsa-miR-101 | 1.72 | 530 | 308 | 6 | 7 | 0.0437 |
| 294 | 6 | 191 | 289 | hsa-miR-148b | 1.41 | 916 | 651 | 6 | 7 | 0.0017 |
| 295 | 6 | 49 | 302 | hsa-miR-222 | 1.69 | 1801 | 3039 | 6 | 7 | 0.0416 |
| 296 | 6 | 200 | 448 | hsa-miR-339 | 2.02 | 677 | 336 | 6 | 7 | 0.0363 |
| 297 | 6 | 35 | 406 | hsa-miR-373* | 4.87 | 961 | 198 | 6 | 7 | 0.027 |
| 298 | 7 | 149 | 392 | hsa-miR-106b | 1.34 | 909 | 680 | 15 | 18 | 0.00775 |
| 299 | 7 | 204 | 419 | hsa-miR-126 | 2.17 | 650 | 1410 | 15 | 18 | 0.00531 |
| 300 | 7 | 266 | 295, 386 | hsa-miR-133a | 2.36 | 387 | 912 | 15 | 18 | 0.00048 |
| 301 | 7 | 218 | 527 | hsa-miR-143 | 2.67 | 7342 | 19616 | 15 | 18 | 7.11e−006 |
| 302 | 7 | 139 | 281 | hsa-miR-145 | 2.05 | 9126 | 18723 | 15 | 18 | 0.000103 |
| 303 | 7 | 10 | 371, 461 | hsa-miR-181a | 1.48 | 1095 | 742 | 15 | 18 | 0.00527 |
| 304 | 7 | 109 | 374 | hsa-miR-192 | 1.68 | 1371 | 2305 | 15 | 18 | 0.0444 |
| 305 | 7 | 170 | 327 | hsa-miR-195 | 2.61 | 386 | 1007 | 15 | 18 | 0.00628 |
| 306 | 7 | 156 | 446 | hsa-miR-200c | 1.72 | 3980 | 2318 | 15 | 18 | 0.015 |
| 307 | 7 | 50 | 364 | hsa-miR-221 | 1.29 | 1094 | 849 | 15 | 18 | 0.021 |
| 308 | 7 | 49 | 302 | hsa-miR-222 | 1.39 | 1873 | 1350 | 15 | 18 | 0.0108 |
| 309 | 7 | 256 | 441, 487 | hsa-miR-26a | 1.84 | 2508 | 4604 | 15 | 18 | 0.00784 |
| 310 | 7 | 260 | 280 | hsa-miR-27b | 2.88 | 732 | 2113 | 15 | 18 | 0.0123 |
| 311 | 7 | 242 | 311 | hsa-miR-30b | 2.24 | 312 | 699 | 15 | 18 | 0.0225 |
| 312 | 7 | 243 | 299, 316 | hsa-miR-30c | 2.58 | 411 | 1058 | 15 | 18 | 0.0127 |
| 313 | 7 | 237 | 480 | hsa-miR-34a | 1.47 | 917 | 626 | 15 | 18 | 0.00156 |
| 314 | 7 | 111 | 389 | hsa-miR-422a | 1.99 | 292 | 583 | 15 | 18 | 8.61e−005 |
| 315 | 7 | 112 | 372 | hsa-miR-422b | 1.9 | 820 | 1559 | 15 | 18 | 0.000311 |
| 316 | 7 | 63 | 557 | hsa-miR-491 | 3.45 | 2924 | 848 | 15 | 18 | 0.00142 |
| 317 | 7 | 83 | 312 | hsa-miR-497 | 2.44 | 609 | 1487 | 15 | 18 | 0.000396 |
| 318 | 7 | 135 | 315 | hsa-miR-565 | 2.23 | 746 | 1668 | 7 | 5 | 0.0385 |
| 319 | 7 | 182 | 304, 317 | hsa-miR-92 | 2.15 | 2463 | 1146 | 15 | 18 | 0.00388 |
| 320 | 8 | 127 | 361 | 11_1 | 4.04 | 190 | 771 | 5 | 8 | 0.00294 |
| 321 | 8 | 172 | 329 | hsa-miR-15b | 3.48 | 226 | 785 | 5 | 8 | 0.00918 |
| 322 | 8 | 75 | 467 | hsa-miR-17-5p | 2.39 | 457 | 1094 | 5 | 8 | 0.0123 |
| 323 | 8 | 18 | 322 | hsa-miR-22 | 2.21 | 2214 | 1001 | 5 | 8 | 0.000838 |
| 324 | 8 | 49 | 302 | hsa-miR-222 | 1.67 | 2139 | 1284 | 5 | 8 | 0.00797 |
| 325 | 8 | 90 | 388 | hsa-miR-25 | 2.46 | 222 | 546 | 5 | 8 | 0.0468 |
| 326 | 8 | 1 | 359 | hsa-miR-320 | 2.39 | 2674 | 1119 | 5 | 8 | 0.0145 |
| 327 | 8 | 14 | 318 | hsa-miR-99a | 1.99 | 906 | 455 | 5 | 8 | 0.00524 |
| 328 | 9 | 115 | 314 | hsa-miR-210 | 9.71 | 1576 | 162 | 9 | 6 | 0.00223 |
| 329 | 9 | 67 | 336 | hsa-miR-23a | 2.74 | 4108 | 1499 | 9 | 6 | 0.00483 |
| 330 | 9 | 244 | 287 | hsa-miR-30a-5p | 3.18 | 2854 | 9062 | 9 | 6 | 0.0262 |
| 331 | 9 | 245 | 431 | hsa-miR-30e-5p | 5.69 | 269 | 1528 | 9 | 6 | 0.00861 |
| 332 | 9 | 141 | 437 | hsa-miR-33 | 1.66 | 914 | 1518 | 9 | 6 | 0.0233 |
| 333 | 9 | 126 | 380 | hsa-miR-331 | 1.48 | 797 | 1177 | 9 | 6 | 0.00961 |
| 334 | 9 | 237 | 480 | hsa-miR-34a | 3.09 | 1755 | 569 | 9 | 6 | 0.00181 |
| 335 | 9 | 135 | 315 | hsa-miR-565 | 3.13 | 391 | 1222 | 9 | 6 | 0.00989 |
| 336 | 10 | 94 | 394 | 69_6_rel | 1.99 | 7217 | 3619 | 32 | 31 | 0.0302 |
| 337 | 10 | 177 | 366, 464 | 72_12 | 1.71 | 4431 | 7580 | 32 | 31 | 0.00444 |
| 338 | 10 | 163 | 402 | 43_7 | 1.54 | 1308 | 851 | 32 | 31 | 0.0303 |
| 339 | 10 | 223 | 320, 393, 559 | hsa-let-7a | 1.51 | 7629 | 5049 | 32 | 31 | 0.0453 |
| 340 | 10 | 45 | 277 | hsa-let-7d | 1.48 | 3200 | 2161 | 32 | 31 | 0.0174 |
| 341 | 10 | 13 | 397 | hsa-miR-100 | 1.78 | 1423 | 2530 | 32 | 31 | 0.0235 |
| 342 | 10 | 184 | 423 | hsa-miR-105 | 1.72 | 3048 | 1770 | 32 | 31 | 0.000305 |
| 343 | 10 | 235 | 358 | hsa-miR-122a | 1.59 | 25769 | 40850 | 32 | 31 | 0.0319 |
| 344 | 10 | 199 | 387 | hsa-miR-125a | 1.63 | 1759 | 2870 | 32 | 31 | 0.0493 |
| 345 | 10 | 198 | 313, 551 | hsa-miR-125b | 1.5 | 8459 | 12694 | 32 | 31 | 0.0359 |
| 346 | 10 | 86 | 417 | hsa-miR-130a | 1.79 | 1497 | 2677 | 32 | 31 | 0.00246 |
| 347 | 10 | 172 | 329 | hsa-miR-15b | 2.64 | 2323 | 880 | 32 | 31 | 0.00583 |
| 348 | 10 | 11 | 456, 540 | hsa-miR-181b | 1.56 | 836 | 535 | 32 | 31 | 0.0281 |
| 349 | 10 | 92 | 410, 432 | hsa-miR-199a | 4.19 | 2825 | 11830 | 32 | 31 | 9.13e−005 |
| 350 | 10 | 157 | 411 | hsa-miR-200b | 1.93 | 299 | 578 | 32 | 31 | 0.00803 |
| 351 | 10 | 175 | 462 | hsa-miR-21 | 2.38 | 9817 | 4126 | 32 | 31 | 0.000181 |
| 352 | 10 | 115 | 314 | hsa-miR-210 | 1.58 | 716 | 453 | 32 | 31 | 0.0356 |
| 353 | 10 | 29 | 439 | hsa-miR-214 | 2.29 | 762 | 1743 | 32 | 31 | 0.00294 |
| 354 | 10 | 18 | 322 | hsa-miR-22 | 1.91 | 6349 | 12109 | 32 | 31 | 8.04e−005 |
| 355 | 10 | 50 | 364 | hsa-miR-221 | 2.63 | 3058 | 1165 | 32 | 31 | 3.61e−005 |
| 356 | 10 | 49 | 302 | hsa-miR-222 | 2.66 | 3953 | 1487 | 32 | 31 | 3.99e−006 |
| 357 | 10 | 126 | 380 | hsa-miR-331 | 1.77 | 2226 | 1260 | 32 | 31 | 4.83e−005 |
| 358 | 10 | 200 | 448 | hsa-miR-339 | 2.02 | 948 | 470 | 32 | 31 | 7.56e−005 |
| 359 | 10 | 229 | 292 | hsa-miR-345 | 1.74 | 731 | 419 | 32 | 31 | 0.000687 |
| 360 | 10 | 112 | 372 | hsa-miR-422b | 2.65 | 924 | 2448 | 32 | 31 | 9.54e−005 |
| 361 | 10 | 51 | 497 | hsa-miR-423 | 1.48 | 1956 | 1325 | 32 | 31 | 0.02 |
| 362 | 10 | 63 | 557 | hsa-miR-491 | 1.58 | 1274 | 808 | 32 | 31 | 0.0164 |
| 363 | 10 | 83 | 312 | hsa-miR-497 | 1.97 | 1578 | 3102 | 32 | 31 | 0.00322 |
| 364 | 10 | 60 | 479 | hsa-miR-638 | 1.7 | 9933 | 5859 | 32 | 31 | 0.0255 |
| 365 | 10 | 123 | 291 | hsa-miR-801 | 2.37 | 3497 | 1474 | 9 | 10 | 0.0218 |
| 366 | 10 | 6 | 471 | hsa-miR-93 | 1.61 | 2804 | 1738 | 32 | 31 | 0.00101 |
| 367 | 10 | 14 | 318 | hsa-miR-99a | 2.25 | 3761 | 8472 | 32 | 31 | 4.85e−005 |
| 368 | 11 | 197 | 444 | 25_6 | 1.65 | 423 | 696 | 25 | 8 | 0.00177 |
| 369 | 11 | 42 | 449 | 43_3 | 3.79 | 136 | 514 | 32 | 8 | 1.74e−007 |
| 370 | 11 | 101 | 475 | 43_30 | 2.61 | 235 | 614 | 32 | 8 | 7.29e−005 |
| 371 | 11 | 145 | 447 | 43_44_rel | 2.07 | 33094 | 68595 | 15 | 8 | 1.57e−005 |
| 372 | 11 | 114 | 530 | 43_6 | 3.71 | 158 | 586 | 32 | 8 | 1.6e−007 |
| 373 | 11 | 183 | 373 | 66_1 | 2.51 | 202 | 507 | 32 | 8 | 0.000793 |
| 374 | 11 | 121 | 502 | 70_14 | 3.68 | 533 | 145 | 32 | 8 | 0.00192 |
| 375 | 11 | 104 | 347 | 72_23 | 9.02 | 528 | 59 | 32 | 8 | 3.87e−005 |
| 376 | 11 | 97 | 357 | 74_26_rel | 4.51 | 801 | 178 | 32 | 8 | 0.00082 |
| 377 | 11 | 33 | 339, 552 | 96_rel | 1.97 | 31327 | 61712 | 15 | 8 | 7.24e−005 |
| 378 | 11 | 125 | 532 | 70_16 | 3.1 | 1952 | 629 | 32 | 8 | 0.0416 |
| 379 | 11 | 163 | 402 | 43_7 | 15.55 | 1237 | 80 | 32 | 8 | 9.16e−013 |
| 380 | 11 | 220 | 475 | hsa-let-7e | 1.64 | 1940 | 3178 | 32 | 8 | 0.0262 |
| 381 | 11 | 226 | 395 | hsa-let-7g | 1.74 | 700 | 1220 | 32 | 8 | 0.032 |
| 382 | 11 | 227 | 297 | hsa-let-7i | 2.06 | 826 | 1705 | 32 | 8 | 0.0262 |
| 383 | 11 | 13 | 397 | hsa-miR-100 | 2.3 | 1350 | 588 | 32 | 8 | 0.0225 |
| 384 | 11 | 48 | 483, 529 | hsa-miR-103 | 3.1 | 7621 | 2456 | 32 | 8 | 0.00744 |
| 385 | 11 | 149 | 392 | hsa-miR-106b | 2.41 | 5988 | 2487 | 32 | 8 | 0.00266 |
| 386 | 11 | 47 | 555 | hsa-miR-107 | 3.73 | 7282 | 1953 | 32 | 8 | 2.53e−005 |
| 387 | 11 | 235 | 358 | hsa-miR-122a | 4.76 | 16654 | 3496 | 32 | 8 | 0.000125 |
| 388 | 11 | 199 | 387 | hsa-miR-125a | 2.43 | 1643 | 3992 | 32 | 8 | 0.0228 |
| 389 | 11 | 198 | 313, 551 | hsa-miR-125b | 1.92 | 6497 | 3381 | 32 | 8 | 0.0338 |
| 390 | 11 | 204 | 419 | hsa-miR-126 | 4.69 | 5513 | 1175 | 32 | 8 | 3.44e−007 |
| 391 | 11 | 266 | 295, 386 | hsa-miR-133a | 2.78 | 198 | 551 | 32 | 8 | 0.00015 |
| 392 | 11 | 250 | 469 | hsa-miR-134 | 3.55 | 160 | 569 | 32 | 8 | 1.6e−006 |
| 393 | 11 | 37 | 391 | hsa-miR-136 | 3.09 | 223 | 690 | 32 | 8 | 0.000128 |
| 394 | 11 | 52 | 370, 548 | hsa-miR-138 | 2.67 | 353 | 943 | 32 | 8 | 0.000287 |
| 395 | 11 | 64 | 402 | hsa-miR-140 | 3.64 | 237 | 863 | 32 | 8 | 7.12e−007 |
| 396 | 11 | 151 | 472 | hsa-miR-141 | 9.89 | 150 | 1486 | 32 | 8 | 3.99e−007 |
| 397 | 11 | 247 | 278 | hsa-miR-142-3p | 3.68 | 158 | 581 | 32 | 8 | 1.76e−008 |
| 398 | 11 | 146 | 325 | hsa-miR-147 | 4.45 | 131 | 582 | 32 | 8 | 1.94e−009 |
| 399 | 11 | 34 | 396 | hsa-miR-151 | 2.08 | 657 | 316 | 32 | 8 | 0.00841 |
| 400 | 11 | 9 | 451 | hsa-miR-181c | 4.33 | 150 | 650 | 32 | 8 | 4.65e−008 |
| 401 | 11 | 240 | 323 | hsa-miR-182* | 2.51 | 218 | 548 | 32 | 8 | 0.000723 |
| 402 | 11 | 179 | 356 | hsa-miR-183 | 3.37 | 154 | 520 | 32 | 8 | 6.26e−005 |
| 403 | 11 | 234 | 450 | hsa-miR-185 | 3.26 | 1719 | 527 | 32 | 8 | 1.98e−007 |
| 404 | 11 | 228 | 435 | hsa-miR-190 | 7.53 | 124 | 931 | 32 | 8 | 5.62e−013 |
| 405 | 11 | 77 | 440 | hsa-miR-191 | 3.31 | 3328 | 1005 | 32 | 8 | 3.57e−006 |
| 406 | 11 | 17 | 442 | hsa-miR-193a | 2.83 | 8688 | 3068 | 32 | 8 | 0.00448 |
| 407 | 11 | 152 | 369 | hsa-miR-200a | 10.92 | 233 | 2542 | 32 | 8 | 1.03e−007 |
| 408 | 11 | 157 | 411 | hsa-miR-200b | 8.22 | 339 | 2789 | 32 | 8 | 1.49e−005 |
| 409 | 11 | 156 | 446 | hsa-miR-200c | 21.12 | 205 | 4328 | 32 | 8 | 1.48e−009 |
| 410 | 11 | 232 | 422 | hsa-miR-206 | 4.32 | 150 | 650 | 32 | 8 | 1.22e−008 |
| 411 | 11 | 18 | 322 | hsa-miR-22 | 2.86 | 4917 | 1722 | 32 | 8 | 5.83e−005 |
| 412 | 11 | 50 | 364 | hsa-miR-221 | 2.24 | 2654 | 1183 | 32 | 8 | 0.0219 |
| 413 | 11 | 49 | 302 | hsa-miR-222 | 1.97 | 3335 | 1689 | 32 | 8 | 0.0236 |
| 414 | 11 | 248 | 415 | hsa-miR-223 | 3.25 | 254 | 827 | 32 | 8 | 3.48e−006 |
| 415 | 11 | 238 | 346, 416 | hsa-miR-24 | 1.7 | 17126 | 10085 | 32 | 8 | 0.000623 |
| 416 | 11 | 256 | 441, 487 | hsa-miR-26a | 1.85 | 11567 | 6269 | 32 | 8 | 0.00239 |
| 417 | 11 | 260 | 280 | hsa-miR-27b | 2.41 | 3665 | 1521 | 32 | 8 | 0.00115 |
| 418 | 11 | 168 | 303, 530 | hsa-miR-29b | 3.77 | 162 | 611 | 32 | 8 | 0.000242 |
| 419 | 11 | 242 | 311 | hsa-miR-30b | 2.18 | 1419 | 650 | 32 | 8 | 0.0425 |
| 420 | 11 | 243 | 299, 316 | hsa-miR-30c | 3.12 | 2834 | 908 | 32 | 8 | 0.00239 |
| 421 | 11 | 241 | 550 | hsa-miR-30d | 5.11 | 3644 | 712 | 32 | 8 | 3.05e−005 |
| 422 | 11 | 245 | 431 | hsa-miR-30e-5p | 22.78 | 2885 | 127 | 32 | 8 | 3.28e−008 |
| 423 | 11 | 132 | 429 | hsa-miR-31 | 2.49 | 398 | 992 | 32 | 8 | 0.0164 |
| 424 | 11 | 1 | 359 | hsa-miR-320 | 2.05 | 3284 | 1601 | 32 | 8 | 0.00963 |
| 425 | 11 | 113 | 538 | hsa-miR-328 | 3.92 | 282 | 1107 | 32 | 8 | 1.48e−005 |
| 426 | 11 | 141 | 437 | hsa-miR-33 | 4.23 | 328 | 1386 | 32 | 8 | 0.000284 |
| 427 | 11 | 126 | 380 | hsa-miR-331 | 1.49 | 1995 | 1341 | 32 | 8 | 0.0311 |
| 428 | 11 | 194 | 337 | hsa-miR-338 | 5.47 | 358 | 1960 | 32 | 8 | 0.00426 |
| 429 | 11 | 200 | 448 | hsa-miR-339 | 1.69 | 947 | 562 | 32 | 8 | 0.0251 |
| 430 | 11 | 208 | 477 | hsa-miR-342 | 3.31 | 1213 | 367 | 32 | 8 | 0.000241 |
| 431 | 11 | 249 | 284 | hsa-miR-346 | 4.61 | 119 | 547 | 32 | 8 | 1.45e−012 |
| 432 | 11 | 237 | 480 | hsa-miR-34a | 2.82 | 4122 | 1462 | 32 | 8 | 5.72e−005 |
| 433 | 11 | 31 | 560 | hsa-miR-368 | 3.27 | 165 | 537 | 32 | 8 | 1.64e−006 |
| 434 | 11 | 128 | 294 | hsa-miR-370 | 1.76 | 480 | 846 | 32 | 8 | 0.0334 |
| 435 | 11 | 35 | 406 | hsa-miR-373* | 2.48 | 215 | 534 | 32 | 8 | 0.0172 |
| 436 | 11 | 273 | 335 | hsa-miR-375 | 5.16 | 130 | 670 | 32 | 8 | 0.000286 |
| 437 | 11 | 105 | 372 | hsa-miR-378 | 1.99 | 273 | 544 | 32 | 8 | 0.00358 |
| 438 | 11 | 46 | 298 | hsa-miR-383 | 3.26 | 154 | 502 | 32 | 8 | 0.0063 |
| 439 | 11 | 213 | 531 | hsa-miR-432 | 2.77 | 183 | 507 | 32 | 8 | 0.00142 |
| 440 | 11 | 264 | 541 | hsa-miR-448 | 5.34 | 110 | 588 | 32 | 8 | 1.49e−012 |
| 441 | 11 | 236 | 413 | hsa-miR-449 | 3.35 | 240 | 802 | 32 | 8 | 0.000619 |
| 442 | 11 | 4 | 430 | hsa-miR-451 | 4.8 | 508 | 106 | 32 | 8 | 0.00261 |
| 443 | 11 | 44 | 547 | hsa-miR-485-5p | 3.11 | 225 | 698 | 32 | 8 | 0.000401 |
| 444 | 11 | 63 | 557 | hsa-miR-491 | 1.96 | 1224 | 2399 | 32 | 8 | 0.0137 |
| 445 | 11 | 54 | 293 | hsa-miR-492 | 2.68 | 237 | 636 | 32 | 8 | 0.000123 |
| 446 | 11 | 214 | 376 | hsa-miR-494 | 11.59 | 2195 | 25438 | 32 | 8 | 4.94e−006 |
| 447 | 11 | 83 | 312 | hsa-miR-497 | 2.35 | 1469 | 626 | 32 | 8 | 0.0259 |
| 448 | 11 | 258 | 428, 443 | hsa-miR-513 | 3.41 | 170 | 579 | 32 | 8 | 1.6e−007 |
| 449 | 11 | 73 | 503 | hsa-miR-518b | 3.13 | 215 | 672 | 32 | 8 | 1.35e−005 |
| 450 | 11 | 210 | 400 | hsa-miR-518c* | 3.08 | 208 | 639 | 32 | 8 | 1.05e−005 |
| 451 | 11 | 206 | 513 | hsa-miR-520d* | 2.94 | 199 | 586 | 32 | 8 | 5.5e−006 |
| 452 | 11 | 60 | 479 | hsa-miR-638 | 6.34 | 7444 | 1174 | 32 | 8 | 7.51e−005 |
| 453 | 11 | 230 | 474, 484, 536 | hsa-miR-7 | 5.86 | 133 | 780 | 25 | 8 | 6.81e−011 |
| 454 | 11 | 36 | 385 | hsa-miR-766 | 5.48 | 556 | 3045 | 9 | 8 | 0.0027 |
| 455 | 11 | 123 | 291 | hsa-miR-801 | 4.36 | 2906 | 667 | 9 | 8 | 0.000329 |
| 456 | 11 | 6 | 471 | hsa-miR-93 | 2.31 | 2427 | 1049 | 32 | 8 | 0.00258 |
| 457 | 11 | 221 | 307 | hsa-miR-98 | 3.58 | 164 | 588 | 32 | 8 | 2.15e−006 |
| 458 | 11 | 14 | 318 | hsa-miR-99a | 5.36 | 3102 | 578 | 32 | 8 | 2.62e−006 |
| 459 | 12 | 183 | 373 | 66_1 | 2.16 | 267 | 577 | 6 | 5 | 0.0338 |
| 460 | 12 | 94 | 394 | 69_6_rel | 3.04 | 4705 | 14302 | 7 | 5 | 0.00616 |
| 461 | 12 | 177 | 366, 464 | 72_12 | 3.84 | 976 | 254 | 7 | 5 | 0.00432 |
| 462 | 12 | 97 | 357 | 74_26_rel | 3.56 | 726 | 2585 | 7 | 5 | 0.00153 |
| 463 | 12 | 223 | 320, 393, 559 | hsa-let-7a | 2.77 | 2021 | 730 | 7 | 5 | 0.0457 |
| 464 | 12 | 225 | 332 | hsa-let-7b | 3.27 | 10403 | 3184 | 7 | 5 | 0.00159 |
| 465 | 12 | 224 | 427 | hsa-let-7c | 2.31 | 3332 | 1441 | 7 | 5 | 0.0481 |
| 466 | 12 | 45 | 277 | hsa-let-7d | 1.53 | 1259 | 822 | 7 | 5 | 0.0366 |
| 467 | 12 | 226 | 395 | hsa-let-7g | 2.38 | 574 | 1368 | 7 | 5 | 0.029 |
| 468 | 12 | 48 | 483, 529 | hsa-miR-103 | 2.89 | 1517 | 525 | 7 | 5 | 0.0185 |
| 469 | 12 | 47 | 555 | hsa-miR-107 | 1.85 | 1209 | 654 | 7 | 5 | 0.0143 |
| 470 | 12 | 199 | 387 | hsa-miR-125a | 2.26 | 1674 | 739 | 7 | 5 | 0.00823 |
| 471 | 12 | 204 | 419 | hsa-miR-126 | 4.91 | 1853 | 377 | 7 | 5 | 0.00705 |
| 472 | 12 | 86 | 417 | hsa-miR-130a | 2.36 | 753 | 1777 | 7 | 5 | 0.0218 |
| 473 | 12 | 37 | 391 | hsa-miR-136 | 2.76 | 229 | 632 | 7 | 5 | 0.0148 |
| 474 | 12 | 64 | 402 | hsa-miR-140 | 2.74 | 282 | 773 | 7 | 5 | 0.00814 |
| 475 | 12 | 218 | 527 | hsa-miR-143 | 2.76 | 3171 | 1151 | 7 | 5 | 0.00247 |
| 476 | 12 | 139 | 281 | hsa-miR-145 | 2.67 | 2951 | 1104 | 7 | 5 | 0.0124 |
| 477 | 12 | 212 | 333 | hsa-miR-149 | 2.46 | 252 | 620 | 7 | 5 | 0.0291 |
| 478 | 12 | 209 | 507 | hsa-miR-150 | 2.31 | 823 | 356 | 7 | 5 | 0.00399 |
| 479 | 12 | 179 | 356 | hsa-miR-183 | 4.33 | 373 | 1613 | 7 | 5 | 0.000878 |
| 480 | 12 | 88 | 310 | hsa-miR-188 | 2.76 | 202 | 558 | 7 | 5 | 0.0258 |
| 481 | 12 | 144 | 346 | hsa-miR-189 | 3.14 | 172 | 540 | 7 | 5 | 0.00623 |
| 482 | 12 | 16 | 488 | hsa-miR-193b | 1.91 | 524 | 1000 | 7 | 5 | 0.0313 |
| 483 | 12 | 92 | 410, 432 | hsa-miR-199a | 2.83 | 2245 | 793 | 7 | 5 | 0.00118 |
| 484 | 12 | 152 | 369 | hsa-miR-200a | 2.06 | 656 | 319 | 7 | 5 | 0.0496 |
| 485 | 12 | 271 | 334 | hsa-miR-202* | 2.66 | 208 | 552 | 7 | 5 | 0.033 |
| 486 | 12 | 67 | 336 | hsa-miR-23a | 4.22 | 2322 | 550 | 7 | 5 | 0.0148 |
| 487 | 12 | 238 | 346, 416 | hsa-miR-24 | 2.6 | 7309 | 2814 | 7 | 5 | 0.0138 |
| 488 | 12 | 256 | 441, 487 | hsa-miR-26a | 3.16 | 4356 | 1378 | 7 | 5 | 0.00738 |
| 489 | 12 | 257 | 409 | hsa-miR-26b | 2.39 | 297 | 707 | 7 | 5 | 0.00161 |
| 490 | 12 | 259 | 481 | hsa-miR-27a | 2.61 | 2288 | 877 | 7 | 5 | 0.043 |
| 491 | 12 | 244 | 287 | hsa-miR-30a-5p | 2.21 | 1233 | 557 | 7 | 5 | 0.0411 |
| 492 | 12 | 243 | 299, 316 | hsa-miR-30c | 2.82 | 710 | 252 | 7 | 5 | 0.0312 |
| 493 | 12 | 241 | 550 | hsa-miR-30d | 2.1 | 1750 | 835 | 7 | 5 | 0.02 |
| 494 | 12 | 113 | 538 | hsa-miR-328 | 2.19 | 566 | 1243 | 7 | 5 | 0.00481 |
| 495 | 12 | 126 | 380 | hsa-miR-331 | 1.6 | 648 | 405 | 7 | 5 | 0.03 |
| 496 | 12 | 254 | 496 | hsa-miR-374 | 2.8 | 200 | 561 | 7 | 5 | 0.00271 |
| 497 | 12 | 51 | 497 | hsa-miR-423 | 2.71 | 1094 | 2969 | 7 | 5 | 0.00534 |
| 498 | 12 | 236 | 413 | hsa-miR-449 | 6.14 | 590 | 3622 | 7 | 5 | 0.00258 |
| 499 | 12 | 252 | 405 | hsa-miR-452 | 2.4 | 310 | 745 | 7 | 5 | 0.0311 |
| 500 | 12 | 44 | 547 | hsa-miR-485-5p | 2.51 | 360 | 903 | 7 | 5 | 0.0191 |
| 501 | 12 | 63 | 557 | hsa-miR-491 | 2.99 | 1066 | 3183 | 7 | 5 | 0.0432 |
| 502 | 12 | 54 | 293 | hsa-miR-492 | 3.02 | 247 | 746 | 7 | 5 | 0.00459 |
| 503 | 12 | 258 | 428, 443 | hsa-miR-513 | 2.92 | 349 | 1021 | 7 | 5 | 0.0121 |
| 504 | 12 | 73 | 503 | hsa-miR-518b | 2.06 | 265 | 544 | 7 | 5 | 0.0321 |
| 505 | 12 | 60 | 479 | hsa-miR-638 | 3.07 | 4676 | 14367 | 7 | 5 | 0.022 |
| 506 | 12 | 14 | 318 | hsa-miR-99a | 2.44 | 786 | 322 | 7 | 5 | 0.000234 |
| 507 | 12 | 80 | 509 | hsa-miR-99b | 1.57 | 543 | 346 | 7 | 5 | 0.0377 |
| 508 | 13 | 94 | 394 | 69_6_rel | 2.09 | 4594 | 9618 | 7 | 14 | 0.000787 |
| 509 | 13 | 97 | 357 | 74_26_rel | 2.26 | 786 | 1778 | 7 | 14 | 0.00139 |
| 510 | 13 | 33 | 339, 552 | 96_rel | 1.51 | 20413 | 13497 | 7 | 13 | 0.0368 |
| 511 | 13 | 87 | 301 | gam11 | 1.54 | 8965 | 13802 | 7 | 14 | 0.0188 |
| 512 | 13 | 226 | 395 | hsa-let-7g | 1.65 | 623 | 1030 | 7 | 14 | 0.00653 |
| 513 | 13 | 199 | 387 | hsa-miR-125a | 1.46 | 1749 | 1200 | 7 | 14 | 0.0451 |
| 514 | 13 | 204 | 419 | hsa-miR-126 | 1.83 | 1920 | 1048 | 7 | 14 | 0.0221 |
| 515 | 13 | 153 | 445 | hsa-miR-132 | 2.86 | 243 | 694 | 7 | 14 | 0.00963 |
| 516 | 13 | 64 | 402 | hsa-miR-140 | 1.97 | 310 | 611 | 7 | 14 | 0.000795 |
| 517 | 13 | 136 | 308 | hsa-miR-198 | 2.14 | 282 | 604 | 7 | 14 | 0.0132 |
| 518 | 13 | 92 | 410, 432 | hsa-miR-199a | 1.39 | 2307 | 1659 | 7 | 14 | 0.0129 |
| 519 | 13 | 152 | 369 | hsa-miR-200a | 1.6 | 708 | 1131 | 7 | 14 | 0.0348 |
| 520 | 13 | 156 | 446 | hsa-miR-200c | 1.88 | 2482 | 4658 | 7 | 14 | 0.00378 |
| 521 | 13 | 203 | 504 | hsa-miR-205 | 15.79 | 293 | 4626 | 7 | 14 | 7.88e−005 |
| 522 | 13 | 50 | 364 | hsa-miR-221 | 1.49 | 2219 | 1486 | 7 | 14 | 0.0306 |
| 523 | 13 | 238 | 346, 416 | hsa-miR-24 | 1.62 | 6903 | 4272 | 7 | 14 | 0.00433 |
| 524 | 13 | 90 | 388 | hsa-miR-25 | 1.74 | 529 | 922 | 7 | 14 | 0.0221 |
| 525 | 13 | 256 | 441, 487 | hsa-miR-26a | 2.11 | 4254 | 2013 | 7 | 14 | 0.00712 |
| 526 | 13 | 257 | 409 | hsa-miR-26b | 1.71 | 327 | 558 | 7 | 14 | 0.000429 |
| 527 | 13 | 169 | 528 | hsa-miR-29c | 2.56 | 216 | 554 | 7 | 14 | 0.0216 |
| 528 | 13 | 116 | 431 | hsa-miR-30e-3p | 2.29 | 236 | 541 | 7 | 14 | 0.00326 |
| 529 | 13 | 237 | 480 | hsa-miR-34a | 1.7 | 1785 | 1048 | 7 | 14 | 0.0222 |
| 530 | 13 | 111 | 389 | hsa-miR-422a | 4.08 | 278 | 1136 | 7 | 14 | 0.00292 |
| 531 | 13 | 112 | 372 | hsa-miR-422b | 3.45 | 447 | 1543 | 7 | 14 | 0.0029 |
| 532 | 13 | 44 | 547 | hsa-miR-485-5p | 1.75 | 393 | 689 | 7 | 14 | 0.0232 |
| 533 | 13 | 267 | 354 | hsa-miR-493-5p | 2.16 | 236 | 509 | 7 | 14 | 0.0426 |
| 534 | 13 | 258 | 428, 443 | hsa-miR-513 | 2.34 | 382 | 893 | 7 | 14 | 0.000419 |
| 535 | 13 | 120 | 522 | hsa-miR-515-3p | 3.36 | 184 | 617 | 7 | 14 | 0.0431 |
| 536 | 13 | 100 | 524 | hsa-miR-524* | 1.95 | 261 | 508 | 7 | 14 | 0.017 |
| 537 | 13 | 135 | 315 | hsa-miR-565 | 1.96 | 1206 | 2363 | 7 | 14 | 0.0207 |
| 538 | 13 | 182 | 304, 317 | hsa-miR-92 | 1.51 | 1450 | 2187 | 7 | 14 | 0.031 |
| 539 | 14 | 225 | 332 | hsa-let-7b | 2.98 | 2087 | 6229 | 5 | 14 | 0.00117 |
| 540 | 14 | 224 | 427 | hsa-let-7c | 1.96 | 959 | 1876 | 5 | 14 | 0.0239 |
| 541 | 14 | 220 | 475 | hsa-let-7e | 1.43 | 1152 | 807 | 5 | 14 | 0.0405 |
| 542 | 14 | 48 | 483, 529 | hsa-miR-103 | 2.03 | 383 | 778 | 5 | 14 | 0.0344 |
| 543 | 14 | 204 | 419 | hsa-miR-126 | 2.35 | 285 | 671 | 5 | 14 | 0.0205 |
| 544 | 14 | 86 | 417 | hsa-miR-130a | 2.92 | 1198 | 410 | 5 | 14 | 0.000825 |
| 545 | 14 | 151 | 472 | hsa-miR-141 | 1.97 | 1016 | 2002 | 5 | 14 | 0.0304 |
| 546 | 14 | 218 | 527 | hsa-miR-143 | 2.03 | 768 | 1560 | 5 | 14 | 0.00451 |
| 547 | 14 | 139 | 281 | hsa-miR-145 | 3.16 | 740 | 2337 | 5 | 14 | 0.000204 |
| 548 | 14 | 179 | 356 | hsa-miR-183 | 4.74 | 1089 | 230 | 5 | 14 | 0.000476 |
| 549 | 14 | 77 | 440 | hsa-miR-191 | 1.57 | 576 | 905 | 5 | 14 | 0.00826 |
| 550 | 14 | 16 | 488 | hsa-miR-193b | 1.76 | 671 | 381 | 5 | 14 | 0.023 |
| 551 | 14 | 92 | 410, 432 | hsa-miR-199a | 1.95 | 539 | 1052 | 5 | 14 | 0.000862 |
| 552 | 14 | 152 | 369 | hsa-miR-200a | 3.03 | 237 | 720 | 5 | 14 | 1.98e−005 |
| 553 | 14 | 157 | 411 | hsa-miR-200b | 2.25 | 341 | 768 | 5 | 14 | 0.0155 |
| 554 | 14 | 156 | 446 | hsa-miR-200c | 2.83 | 1097 | 3101 | 5 | 14 | 0.000225 |
| 555 | 14 | 203 | 504 | hsa-miR-205 | 12.33 | 262 | 3236 | 5 | 14 | 0.00168 |
| 556 | 14 | 115 | 314 | hsa-miR-210 | 1.79 | 814 | 455 | 5 | 14 | 0.00672 |
| 557 | 14 | 29 | 439 | hsa-miR-214 | 1.62 | 716 | 443 | 5 | 14 | 0.0335 |
| 558 | 14 | 67 | 336 | hsa-miR-23a | 3.99 | 396 | 1580 | 5 | 14 | 0.000688 |
| 559 | 14 | 66 | 306 | hsa-miR-23b | 2.36 | 589 | 1392 | 5 | 14 | 0.0122 |
| 560 | 14 | 90 | 388 | hsa-miR-25 | 1.81 | 330 | 598 | 5 | 14 | 0.0257 |
| 561 | 14 | 259 | 481 | hsa-miR-27a | 2.21 | 594 | 1310 | 5 | 14 | 0.00259 |
| 562 | 14 | 61 | 286 | hsa-miR-296 | 2.7 | 1672 | 618 | 5 | 14 | 0.000307 |
| 563 | 14 | 244 | 287 | hsa-miR-30a-5p | 2.12 | 401 | 851 | 5 | 14 | 0.0378 |
| 564 | 14 | 241 | 550 | hsa-miR-30d | 1.95 | 569 | 1111 | 5 | 14 | 0.00943 |
| 565 | 14 | 113 | 538 | hsa-miR-328 | 2.08 | 834 | 401 | 5 | 14 | 0.031 |
| 566 | 14 | 141 | 437 | hsa-miR-33 | 3.39 | 781 | 230 | 5 | 14 | 0.000938 |
| 567 | 14 | 237 | 480 | hsa-miR-34a | 1.47 | 981 | 667 | 5 | 14 | 0.0343 |
| 568 | 14 | 35 | 406 | hsa-miR-373* | 1.68 | 514 | 305 | 5 | 14 | 0.0056 |
| 569 | 14 | 51 | 497 | hsa-miR-423 | 2.29 | 1988 | 868 | 5 | 14 | 0.000542 |
| 570 | 14 | 236 | 413 | hsa-miR-449 | 5.32 | 2456 | 462 | 5 | 14 | 0.000252 |
| 571 | 14 | 63 | 557 | hsa-miR-491 | 2.31 | 2146 | 931 | 5 | 14 | 0.00277 |
| 572 | 14 | 54 | 293 | hsa-miR-492 | 2 | 517 | 258 | 5 | 14 | 0.000125 |
| 573 | 14 | 135 | 315 | hsa-miR-565 | 2.56 | 593 | 1518 | 5 | 14 | 0.00703 |
| 574 | 14 | 60 | 479 | hsa-miR-638 | 2.26 | 10174 | 4502 | 5 | 14 | 0.00344 |
| 575 | 14 | 6 | 471 | hsa-miR-93 | 1.91 | 467 | 890 | 5 | 14 | 0.0135 |
| 576 | 14 | 14 | 318 | hsa-miR-99a | 2.85 | 237 | 676 | 5 | 14 | 5.5e−008 |
| 577 | 15 | 196 | 508 | 15_rel | 1.58 | 47997 | 30355 | 37 | 13 | 0.0145 |
| 578 | 15 | 197 | 444 | 25_6 | 1.3 | 505 | 389 | 24 | 14 | 0.0353 |
| 579 | 15 | 219 | 497 | 27_3 | 1.44 | 1331 | 927 | 24 | 15 | 0.0203 |
| 580 | 15 | 94 | 394 | 69_6_rel | 1.79 | 10820 | 6044 | 37 | 13 | 0.00479 |
| 581 | 15 | 40 | 553 | 72_4 | 1.61 | 1438 | 896 | 37 | 13 | 0.049 |
| 582 | 15 | 87 | 301 | gam11 | 2.19 | 16155 | 7394 | 37 | 13 | 0.00674 |
| 583 | 15 | 163 | 402 | 43_7 | 1.69 | 579 | 981 | 37 | 13 | 0.0311 |
| 584 | 15 | 223 | 320, 393, 559 | hsa-let-7a | 2.31 | 1859 | 4287 | 38 | 14 | 0.000731 |
| 585 | 15 | 224 | 427 | hsa-let-7c | 1.76 | 3687 | 6498 | 38 | 14 | 0.000671 |
| 586 | 15 | 226 | 395 | hsa-let-7g | 1.58 | 995 | 631 | 38 | 14 | 0.015 |
| 587 | 15 | 184 | 423 | hsa-miR-105 | 2.26 | 376 | 851 | 38 | 14 | 0.00158 |
| 588 | 15 | 149 | 392 | hsa-miR-106b | 1.32 | 1098 | 832 | 38 | 14 | 0.0179 |
| 589 | 15 | 199 | 387 | hsa-miR-125a | 1.54 | 1548 | 2392 | 38 | 14 | 0.0131 |
| 590 | 15 | 198 | 313, 551 | hsa-miR-125b | 1.55 | 3526 | 5483 | 38 | 15 | 0.00321 |
| 591 | 15 | 204 | 419 | hsa-miR-126 | 4.5 | 1293 | 5818 | 38 | 14 | 1.73e−008 |
| 592 | 15 | 89 | 419 | hsa-miR-126* | 2.08 | 856 | 411 | 38 | 15 | 6.95e−005 |
| 593 | 15 | 86 | 417 | hsa-miR-130a | 1.56 | 1056 | 1643 | 38 | 15 | 0.00922 |
| 594 | 15 | 52 | 370, 548 | hsa-miR-138 | 1.5 | 539 | 360 | 38 | 14 | 0.0122 |
| 595 | 15 | 64 | 402 | hsa-miR-140 | 1.57 | 595 | 380 | 38 | 14 | 0.0165 |
| 596 | 15 | 151 | 472 | hsa-miR-141 | 2.14 | 3955 | 1850 | 38 | 15 | 2.56e−005 |
| 597 | 15 | 218 | 527 | hsa-miR-143 | 2.23 | 2116 | 4719 | 38 | 14 | 0.0471 |
| 598 | 15 | 139 | 281 | hsa-miR-145 | 1.51 | 3265 | 4945 | 38 | 14 | 0.0388 |
| 599 | 15 | 172 | 329 | hsa-miR-15b | 1.73 | 506 | 875 | 38 | 15 | 0.00509 |
| 600 | 15 | 173 | 360, 468 | hsa-miR-16 | 2.99 | 1578 | 4726 | 38 | 14 | 1.18e−005 |
| 601 | 15 | 11 | 456, 540 | hsa-miR-181b | 1.31 | 681 | 518 | 38 | 15 | 0.018 |
| 602 | 15 | 77 | 440 | hsa-miR-191 | 1.45 | 1677 | 1161 | 38 | 15 | 0.00668 |
| 603 | 15 | 130 | 440 | hsa-miR-191* | 1.83 | 579 | 316 | 37 | 13 | 0.0321 |
| 604 | 15 | 17 | 442 | hsa-miR-193a | 1.48 | 1202 | 1784 | 38 | 15 | 0.0083 |
| 605 | 15 | 16 | 488 | hsa-miR-193b | 1.9 | 857 | 452 | 38 | 15 | 0.000484 |
| 606 | 15 | 170 | 327 | hsa-miR-195 | 2.78 | 329 | 915 | 38 | 15 | 4.08e−006 |
| 607 | 15 | 136 | 308 | hsa-miR-198 | 1.96 | 506 | 258 | 38 | 15 | 0.00191 |
| 608 | 15 | 162 | 410, 432, 473 | hsa-miR-199a* | 2.05 | 288 | 590 | 38 | 15 | 0.00678 |
| 609 | 15 | 152 | 369 | hsa-miR-200a | 2.07 | 907 | 438 | 38 | 14 | 0.0117 |
| 610 | 15 | 157 | 411 | hsa-miR-200b | 1.71 | 1330 | 778 | 38 | 15 | 0.00395 |
| 611 | 15 | 156 | 446 | hsa-miR-200c | 2.18 | 5138 | 2360 | 38 | 15 | 5.84e−005 |
| 612 | 15 | 203 | 504 | hsa-miR-205 | 3.25 | 1829 | 563 | 38 | 15 | 0.017 |
| 613 | 15 | 175 | 462 | hsa-miR-21 | 1.91 | 5646 | 2956 | 38 | 15 | 0.00905 |
| 614 | 15 | 115 | 314 | hsa-miR-210 | 2.81 | 949 | 338 | 38 | 15 | 3.53e−010 |
| 615 | 15 | 159 | 494 | hsa-miR-216 | 1.74 | 532 | 306 | 38 | 15 | 0.00273 |
| 616 | 15 | 67 | 336 | hsa-miR-23a | 1.5 | 2603 | 3897 | 38 | 15 | 0.02 |
| 617 | 15 | 238 | 346, 416 | hsa-miR-24 | 1.4 | 6179 | 8630 | 38 | 15 | 0.00963 |
| 618 | 15 | 90 | 388 | hsa-miR-25 | 2.01 | 847 | 422 | 38 | 15 | 0.000125 |
| 619 | 15 | 256 | 441, 487 | hsa-miR-26a | 2.29 | 3182 | 7274 | 38 | 15 | 3.46e−006 |
| 620 | 15 | 259 | 481 | hsa-miR-27a | 2.07 | 2283 | 4723 | 38 | 15 | 1.54e−005 |
| 621 | 15 | 260 | 280 | hsa-miR-27b | 2.07 | 728 | 1505 | 38 | 15 | 0.000456 |
| 622 | 15 | 242 | 311 | hsa-miR-30b | 3.04 | 339 | 1030 | 38 | 15 | 4.9e−006 |
| 623 | 15 | 243 | 299, 316 | hsa-miR-30c | 2.3 | 650 | 1497 | 38 | 15 | 3.73e−005 |
| 624 | 15 | 132 | 429 | hsa-miR-31 | 2.61 | 1144 | 437 | 38 | 15 | 0.000426 |
| 625 | 15 | 1 | 359 | hsa-miR-320 | 1.3 | 2511 | 1930 | 38 | 15 | 0.00987 |
| 626 | 15 | 57 | 498 | hsa-miR-34c | 2.61 | 313 | 816 | 38 | 15 | 5.68e−005 |
| 627 | 15 | 128 | 294 | hsa-miR-370 | 2.15 | 863 | 402 | 38 | 15 | 0.000128 |
| 628 | 15 | 111 | 389 | hsa-miR-422a | 2.64 | 633 | 240 | 38 | 14 | 0.00171 |
| 629 | 15 | 112 | 372 | hsa-miR-422b | 2.25 | 958 | 426 | 38 | 15 | 0.00256 |
| 630 | 15 | 51 | 497 | hsa-miR-423 | 1.33 | 1993 | 1504 | 38 | 14 | 0.0221 |
| 631 | 15 | 4 | 430 | hsa-miR-451 | 6.09 | 127 | 774 | 23 | 13 | 2.19e−005 |
| 632 | 15 | 252 | 405 | hsa-miR-452 | 1.56 | 541 | 346 | 38 | 15 | 0.0139 |
| 633 | 15 | 44 | 547 | hsa-miR-485-5p | 2.33 | 703 | 302 | 38 | 15 | 0.000108 |
| 634 | 15 | 63 | 557 | hsa-miR-491 | 1.49 | 2149 | 1444 | 38 | 15 | 0.043 |
| 635 | 15 | 83 | 312 | hsa-miR-497 | 1.99 | 673 | 1341 | 38 | 15 | 9.8e−008 |
| 636 | 15 | 43 | 418 | hsa-miR-504 | 1.53 | 587 | 384 | 38 | 15 | 0.0168 |
| 637 | 15 | 258 | 428, 443 | hsa-miR-513 | 1.78 | 952 | 535 | 38 | 15 | 0.012 |
| 638 | 15 | 103 | 342 | hsa-miR-525 | 2.34 | 530 | 227 | 38 | 15 | 9.33e−005 |
| 639 | 15 | 135 | 315 | hsa-miR-565 | 1.65 | 2420 | 1464 | 37 | 13 | 0.0154 |
| 640 | 15 | 6 | 471 | hsa-miR-93 | 1.6 | 1632 | 1017 | 38 | 15 | 0.00414 |
| 641 | 15 | 14 | 318 | hsa-miR-99a | 1.7 | 1040 | 1771 | 38 | 15 | 0.000318 |
| 642 | 15 | 131 | 430 | 6_11 | 3.71 | 343 | 1272 | 24 | 15 | 2.45e−005 |
| 643 | 16 | 149 | 392 | hsa-miR-106b | 1.83 | 1165 | 636 | 20 | 6 | 0.00024 |
| 644 | 16 | 199 | 387 | hsa-miR-125a | 2.16 | 1229 | 2655 | 20 | 6 | 0.00456 |
| 645 | 16 | 198 | 313, 551 | hsa-miR-125b | 1.93 | 2865 | 5531 | 20 | 6 | 0.00505 |
| 646 | 16 | 52 | 370, 548 | hsa-miR-138 | 1.46 | 531 | 364 | 20 | 6 | 0.0281 |
| 647 | 16 | 151 | 472 | hsa-miR-141 | 2.04 | 4583 | 2246 | 20 | 6 | 0.00271 |
| 648 | 16 | 179 | 356 | hsa-miR-183 | 3.1 | 957 | 309 | 20 | 6 | 0.0039 |
| 649 | 16 | 170 | 327 | hsa-miR-195 | 2.14 | 240 | 514 | 20 | 6 | 0.0489 |
| 650 | 16 | 92 | 410, 432 | hsa-miR-199a | 2.12 | 1619 | 3428 | 20 | 6 | 0.00303 |
| 651 | 16 | 162 | 410, 432, 473 | hsa-miR-199a* | 2.92 | 225 | 656 | 20 | 6 | 0.0133 |
| 652 | 16 | 29 | 439 | hsa-miR-214 | 1.63 | 884 | 1439 | 20 | 6 | 0.047 |
| 653 | 16 | 50 | 364 | hsa-miR-221 | 1.77 | 1447 | 2558 | 20 | 6 | 0.0457 |
| 654 | 16 | 67 | 336 | hsa-miR-23a | 1.94 | 2209 | 4293 | 20 | 6 | 0.0143 |
| 655 | 16 | 90 | 388 | hsa-miR-25 | 1.69 | 828 | 489 | 20 | 6 | 0.0355 |
| 656 | 16 | 256 | 441, 487 | hsa-miR-26a | 1.65 | 2880 | 4742 | 20 | 6 | 0.0437 |
| 657 | 16 | 113 | 538 | hsa-miR-328 | 1.65 | 827 | 501 | 20 | 6 | 0.0486 |
| 658 | 16 | 194 | 337 | hsa-miR-338 | 2.76 | 201 | 555 | 20 | 6 | 0.0322 |
| 659 | 16 | 229 | 292 | hsa-miR-345 | 2.32 | 612 | 264 | 20 | 6 | 0.000937 |
| 660 | 16 | 51 | 497 | hsa-miR-423 | 1.55 | 2151 | 1385 | 20 | 6 | 0.02 |
| 661 | 16 | 63 | 557 | hsa-miR-491 | 1.96 | 2586 | 1323 | 20 | 6 | 0.0318 |
| 662 | 16 | 83 | 312 | hsa-miR-497 | 1.45 | 569 | 823 | 20 | 6 | 0.025 |
| 663 | 16 | 6 | 471 | hsa-miR-93 | 1.8 | 1746 | 971 | 20 | 6 | 0.024 |
| 664 | 17 | 134 | 436 | 24_5_rel | 1.55 | 34484 | 53612 | 10 | 7 | 0.00297 |
| 665 | 17 | 193 | 398 | 93_3_rel | 23.68 | 1273 | 54 | 10 | 7 | 0.0215 |
| 666 | 17 | 2 | 399 | hsa-miR-106a | 2.11 | 949 | 450 | 20 | 11 | 0.00673 |
| 667 | 17 | 86 | 417 | hsa-miR-130a | 1.74 | 968 | 555 | 20 | 11 | 0.0109 |
| 668 | 17 | 153 | 445 | hsa-miR-132 | 2.06 | 304 | 624 | 20 | 11 | 0.0214 |
| 669 | 17 | 139 | 281 | hsa-miR-145 | 1.59 | 1955 | 3112 | 20 | 11 | 0.0484 |
| 670 | 17 | 209 | 507 | hsa-miR-150 | 1.77 | 451 | 798 | 20 | 11 | 0.0269 |
| 671 | 17 | 75 | 467 | hsa-miR-17-5p | 2.02 | 741 | 367 | 20 | 11 | 0.0081 |
| 672 | 17 | 179 | 356 | hsa-miR-183 | 2.82 | 772 | 273 | 20 | 11 | 0.000285 |
| 673 | 17 | 130 | 440 | hsa-miR-191* | 2.47 | 311 | 767 | 19 | 11 | 0.00744 |
| 674 | 17 | 157 | 411 | hsa-miR-200b | 1.69 | 1195 | 708 | 20 | 11 | 0.03 |
| 675 | 17 | 91 | 368 | hsa-miR-324-3p | 1.61 | 427 | 686 | 20 | 11 | 0.0237 |
| 676 | 17 | 111 | 389 | hsa-miR-422a | 2.47 | 391 | 964 | 20 | 11 | 0.0111 |
| 677 | 17 | 112 | 372 | hsa-miR-422b | 2.12 | 594 | 1261 | 20 | 11 | 0.0128 |
| 678 | 17 | 51 | 497 | hsa-miR-423 | 1.31 | 1673 | 1276 | 20 | 11 | 0.0329 |
| 679 | 17 | 236 | 413 | hsa-miR-449 | 2.15 | 1319 | 614 | 20 | 11 | 0.0301 |
| 680 | 17 | 214 | 376 | hsa-miR-494 | 2.21 | 7530 | 3406 | 20 | 11 | 0.0121 |
| 681 | 17 | 120 | 522 | hsa-miR-515-3p | 3.32 | 199 | 659 | 20 | 11 | 0.00364 |
| 682 | 17 | 14 | 318 | hsa-miR-99a | 1.51 | 645 | 972 | 20 | 11 | 0.00512 |
| 683 | 18 | 223 | 320, 393, 559 | hsa-let-7a | 2.41 | 938 | 2257 | 11 | 6 | 0.0452 |
| 684 | 18 | 224 | 427 | hsa-let-7c | 1.86 | 2350 | 4367 | 11 | 6 | 0.0252 |
| 685 | 18 | 226 | 395 | hsa-let-7g | 1.54 | 940 | 611 | 11 | 6 | 0.0327 |
| 686 | 18 | 199 | 387 | hsa-miR-125a | 1.76 | 1283 | 2261 | 11 | 6 | 0.00302 |
| 687 | 18 | 198 | 313, 551 | hsa-miR-125b | 1.55 | 2904 | 4499 | 11 | 6 | 0.019 |
| 688 | 18 | 34 | 396 | hsa-miR-151 | 1.67 | 509 | 305 | 11 | 6 | 0.000894 |
| 689 | 18 | 205 | 367 | hsa-miR-187 | 2.22 | 531 | 239 | 11 | 6 | 0.037 |
| 690 | 18 | 92 | 410, 432 | hsa-miR-199a | 1.64 | 1748 | 2870 | 11 | 6 | 0.00433 |
| 691 | 18 | 162 | 410, 432, 473 | hsa-miR-199a* | 2.73 | 225 | 615 | 11 | 6 | 0.0116 |
| 692 | 18 | 159 | 494 | hsa-miR-216 | 2.05 | 633 | 309 | 11 | 6 | 0.0217 |
| 693 | 18 | 67 | 336 | hsa-miR-23a | 1.78 | 1993 | 3548 | 11 | 6 | 0.0369 |
| 694 | 18 | 90 | 388 | hsa-miR-25 | 1.78 | 819 | 461 | 11 | 6 | 0.000965 |
| 695 | 18 | 257 | 409 | hsa-miR-26b | 1.47 | 503 | 343 | 11 | 6 | 0.0198 |
| 696 | 18 | 91 | 368 | hsa-miR-324-3p | 1.83 | 745 | 406 | 11 | 6 | 0.0386 |
| 697 | 18 | 98 | 368 | hsa-miR-324-5p | 1.63 | 605 | 371 | 11 | 6 | 0.0457 |
| 698 | 18 | 214 | 376 | hsa-miR-494 | 2.5 | 3554 | 8897 | 11 | 6 | 0.00845 |
| 699 | 19 | 196 | 508 | 15_rel | 1.69 | 44684 | 26384 | 5 | 5 | 0.0182 |
| 700 | 19 | 20 | 452 | 24_3 | 3.35 | 156 | 522 | 5 | 5 | 0.0397 |
| 701 | 19 | 94 | 394 | 69_6_rel | 5.07 | 3223 | 635 | 5 | 5 | 6.91e−005 |
| 702 | 19 | 40 | 553 | 72_4 | 6.9 | 784 | 114 | 5 | 5 | 0.00274 |
| 703 | 19 | 87 | 301 | gam11 | 2.59 | 8605 | 3319 | 5 | 5 | 0.0196 |
| 704 | 19 | 163 | 402 | 43_7 | 4.52 | 133 | 600 | 5 | 5 | 0.00538 |
| 705 | 19 | 225 | 332 | hsa-let-7b | 3.87 | 6450 | 24976 | 5 | 5 | 0.0481 |
| 706 | 19 | 220 | 475 | hsa-let-7e | 2.4 | 2796 | 1163 | 5 | 5 | 0.00354 |
| 707 | 19 | 13 | 397 | hsa-miR-100 | 7.76 | 572 | 4438 | 5 | 5 | 0.0205 |
| 708 | 19 | 184 | 423 | hsa-miR-105 | 3.8 | 1900 | 500 | 5 | 5 | 0.0158 |
| 709 | 19 | 149 | 392 | hsa-miR-106b | 4.36 | 1139 | 261 | 5 | 5 | 0.00149 |
| 710 | 19 | 199 | 387 | hsa-miR-125a | 1.84 | 2649 | 4864 | 5 | 5 | 0.0351 |
| 711 | 19 | 198 | 313, 551 | hsa-miR-125b | 6 | 5664 | 33967 | 5 | 5 | 0.00578 |
| 712 | 19 | 151 | 472 | hsa-miR-141 | 19.37 | 1138 | 59 | 5 | 5 | 0.00103 |
| 713 | 19 | 218 | 527 | hsa-miR-143 | 3.25 | 2015 | 6552 | 5 | 5 | 0.0157 |
| 714 | 19 | 139 | 281 | hsa-miR-145 | 6.01 | 1880 | 11306 | 5 | 5 | 0.00591 |
| 715 | 19 | 173 | 360, 468 | hsa-miR-16 | 2.18 | 2219 | 1017 | 5 | 5 | 0.0333 |
| 716 | 19 | 75 | 467 | hsa-miR-17-5p | 5.58 | 1122 | 201 | 5 | 5 | 0.000753 |
| 717 | 19 | 10 | 371, 461 | hsa-miR-181a | 2.49 | 824 | 331 | 5 | 5 | 0.0241 |
| 718 | 19 | 170 | 327 | hsa-miR-195 | 25.43 | 114 | 2906 | 5 | 5 | 0.000201 |
| 719 | 19 | 162 | 410, 432, 473 | hsa-miR-199a* | 5.15 | 527 | 2715 | 5 | 5 | 0.00799 |
| 720 | 19 | 152 | 369 | hsa-miR-200a | 16.3 | 1250 | 77 | 5 | 5 | 0.000182 |
| 721 | 19 | 156 | 446 | hsa-miR-200c | 63.94 | 6325 | 99 | 5 | 5 | 4.13e−007 |
| 722 | 19 | 203 | 504 | hsa-miR-205 | 13.29 | 706 | 53 | 5 | 5 | 0.000492 |
| 723 | 19 | 175 | 462 | hsa-miR-21 | 5.05 | 6437 | 1274 | 5 | 5 | 0.0132 |
| 724 | 19 | 90 | 388 | hsa-miR-25 | 3.12 | 602 | 193 | 5 | 5 | 0.0181 |
| 725 | 19 | 256 | 441, 487 | hsa-miR-26a | 2.51 | 5257 | 13199 | 5 | 5 | 0.00339 |
| 726 | 19 | 61 | 286 | hsa-miR-296 | 3.49 | 784 | 225 | 5 | 5 | 0.0124 |
| 727 | 19 | 241 | 550 | hsa-miR-30d | 1.91 | 2095 | 1096 | 5 | 5 | 0.0258 |
| 728 | 19 | 208 | 477 | hsa-miR-342 | 1.72 | 1229 | 716 | 5 | 5 | 0.0105 |
| 729 | 19 | 51 | 497 | hsa-miR-423 | 1.54 | 893 | 581 | 5 | 5 | 0.0372 |
| 730 | 19 | 63 | 557 | hsa-miR-491 | 3.42 | 1135 | 332 | 5 | 5 | 0.000566 |
| 731 | 19 | 83 | 312 | hsa-miR-497 | 6.52 | 431 | 2806 | 5 | 5 | 0.00149 |
| 732 | 19 | 135 | 315 | hsa-miR-565 | 3.78 | 1187 | 314 | 5 | 5 | 0.00439 |
| 733 | 19 | 60 | 479 | hsa-miR-638 | 3.37 | 3596 | 1068 | 5 | 5 | 3.17e−005 |
| 734 | 19 | 14 | 318 | hsa-miR-99a | 5.92 | 2440 | 14434 | 5 | 5 | 0.00909 |
| 735 | 20 | 149 | 392 | hsa-miR-106b | 1.43 | 1407 | 987 | 8 | 15 | 0.0283 |
| 736 | 20 | 204 | 419 | hsa-miR-126 | 1.68 | 1475 | 879 | 8 | 15 | 0.013 |
| 737 | 20 | 52 | 370, 548 | hsa-miR-138 | 1.96 | 976 | 499 | 8 | 15 | 0.0439 |
| 738 | 20 | 218 | 527 | hsa-miR-143 | 8.28 | 33645 | 4063 | 8 | 15 | 0.0452 |
| 739 | 20 | 191 | 289 | hsa-miR-148b | 1.35 | 518 | 701 | 8 | 15 | 0.0209 |
| 740 | 20 | 212 | 333 | hsa-miR-149 | 1.65 | 1179 | 1945 | 8 | 15 | 0.00818 |
| 741 | 20 | 192 | 470 | hsa-miR-152 | 1.38 | 697 | 964 | 8 | 15 | 0.00896 |
| 742 | 20 | 253 | 499 | hsa-miR-155 | 1.4 | 534 | 745 | 8 | 15 | 0.0437 |
| 743 | 20 | 173 | 360, 468 | hsa-miR-16 | 1.81 | 2964 | 1635 | 8 | 15 | 0.0248 |
| 744 | 20 | 75 | 467 | hsa-miR-17-5p | 1.49 | 1464 | 985 | 8 | 15 | 0.0151 |
| 745 | 20 | 251 | 282, 495 | hsa-miR-19b | 2.23 | 1230 | 550 | 8 | 15 | 0.012 |
| 746 | 20 | 90 | 388 | hsa-miR-25 | 3.36 | 687 | 205 | 8 | 15 | 0.0406 |
| 747 | 20 | 259 | 481 | hsa-miR-27a | 1.76 | 3442 | 1959 | 8 | 15 | 0.0339 |
| 748 | 20 | 1 | 359 | hsa-miR-320 | 1.45 | 4240 | 6136 | 8 | 15 | 0.0404 |
| 749 | 20 | 158 | 378 | hsa-miR-429 | 1.46 | 398 | 579 | 8 | 15 | 0.0302 |
| 750 | 20 | 44 | 547 | hsa-miR-485-5p | 3.7 | 716 | 194 | 8 | 15 | 0.0419 |
| 751 | 20 | 214 | 376 | hsa-miR-494 | 1.88 | 8565 | 16061 | 8 | 15 | 0.023 |
| 752 | 20 | 118 | 342 | hsa-miR-525* | 2.02 | 523 | 1056 | 8 | 15 | 0.0243 |
| 753 | 20 | 107 | 343, 506, 515, | hsa-miR-526c | 3.54 | 535 | 151 | 8 | 15 | 0.0194 |
| | | | 520, 521, 526 | | | | | | | |
| 754 | 20 | 230 | 474, 484, 536 | hsa-miR-7 | 1.55 | 619 | 961 | 8 | 15 | 0.0373 |
| 755 | 21 | 127 | 361 | 11_1 | 1.97 | 1092 | 554 | 30 | 33 | 0.00249 |
| 756 | 21 | 196 | 508 | 15_rel | 1.47 | 33178 | 48678 | 30 | 33 | 0.0403 |
| 757 | 21 | 197 | 444 | 25_6 | 1.47 | 461 | 677 | 30 | 33 | 8.56e−006 |
| 758 | 21 | 101 | 475 | 43_30 | 1.45 | 454 | 656 | 30 | 33 | 0.00011 |
| 759 | 21 | 129 | 548 | 43_39 | 1.49 | 1489 | 2215 | 30 | 33 | 0.00289 |
| 760 | 21 | 183 | 373 | 66_1 | 1.3 | 666 | 864 | 30 | 33 | 0.0222 |
| 761 | 21 | 121 | 502 | 70_14 | 1.35 | 651 | 483 | 30 | 33 | 0.0489 |
| 762 | 21 | 87 | 301 | gam11 | 4 | 2057 | 515 | 30 | 33 | 0.0284 |
| 763 | 21 | 227 | 297 | hsa-let-7i | 1.22 | 2085 | 1710 | 30 | 33 | 0.0165 |
| 764 | 21 | 89 | 419 | hsa-miR-126* | 1.42 | 502 | 711 | 30 | 33 | 0.00204 |
| 765 | 21 | 266 | 295, 386 | hsa-miR-133a | 1.82 | 1155 | 2104 | 30 | 33 | 0.000224 |
| 766 | 21 | 265 | 533 | hsa-miR-133b | 1.62 | 1269 | 2054 | 30 | 33 | 0.00123 |
| 767 | 21 | 180 | 486 | hsa-miR-135b | 1.49 | 415 | 620 | 30 | 33 | 0.00168 |
| 768 | 21 | 52 | 370, 548 | hsa-miR-138 | 1.49 | 509 | 761 | 30 | 33 | 0.00907 |
| 769 | 21 | 64 | 402 | hsa-miR-140 | 1.56 | 625 | 973 | 30 | 33 | 0.0034 |
| 770 | 21 | 151 | 472 | hsa-miR-141 | 2.13 | 3588 | 1686 | 30 | 33 | 5.4e−005 |
| 771 | 21 | 139 | 281 | hsa-miR-145 | 1.26 | 32718 | 41138 | 30 | 33 | 0.0448 |
| 772 | 21 | 190 | 458 | hsa-miR-148a | 1.3 | 532 | 410 | 30 | 33 | 0.016 |
| 773 | 21 | 34 | 396 | hsa-miR-151 | 1.36 | 977 | 719 | 30 | 33 | 0.000472 |
| 774 | 21 | 205 | 367 | hsa-miR-187 | 1.33 | 399 | 530 | 30 | 33 | 0.00804 |
| 775 | 21 | 93 | 473 | hsa-miR-199b | 1.42 | 408 | 577 | 30 | 33 | 0.0365 |
| 776 | 21 | 251 | 282, 495 | hsa-miR-19b | 1.88 | 1003 | 533 | 30 | 33 | 0.0349 |
| 777 | 21 | 152 | 369 | hsa-miR-200a | 2.57 | 699 | 272 | 30 | 33 | 0.00255 |
| 778 | 21 | 156 | 446 | hsa-miR-200c | 2.4 | 5592 | 2327 | 30 | 33 | 1.04e−008 |
| 779 | 21 | 271 | 334 | hsa-miR-202* | 1.36 | 632 | 857 | 30 | 33 | 0.0121 |
| 780 | 21 | 203 | 504 | hsa-miR-205 | 1.7 | 8162 | 4796 | 30 | 33 | 0.00565 |
| 781 | 21 | 268 | 401, 425 | hsa-miR-218 | 1.31 | 526 | 690 | 30 | 33 | 0.0302 |
| 782 | 21 | 248 | 415 | hsa-miR-223 | 1.29 | 465 | 601 | 30 | 33 | 0.0137 |
| 783 | 21 | 238 | 346, 416 | hsa-miR-24 | 1.3 | 10903 | 14194 | 30 | 33 | 0.0203 |
| 784 | 21 | 256 | 441, 487 | hsa-miR-26a | 1.44 | 7189 | 10320 | 30 | 33 | 0.00617 |
| 785 | 21 | 259 | 481 | hsa-miR-27a | 1.61 | 2896 | 4675 | 30 | 33 | 0.00501 |
| 786 | 21 | 167 | 556 | hsa-miR-29a | 3.76 | 616 | 164 | 30 | 33 | 0.00666 |
| 787 | 21 | 244 | 287 | hsa-miR-30a-5p | 3.02 | 504 | 167 | 30 | 33 | 0.0211 |
| 788 | 21 | 241 | 550 | hsa-miR-30d | 1.27 | 2342 | 1838 | 30 | 33 | 0.0253 |
| 789 | 21 | 245 | 431 | hsa-miR-30e-5p | 1.52 | 2709 | 1778 | 30 | 33 | 0.00058 |
| 790 | 21 | 91 | 368 | hsa-miR-324-3p | 1.2 | 1037 | 1244 | 30 | 33 | 0.0224 |
| 791 | 21 | 113 | 538 | hsa-miR-328 | 1.85 | 869 | 1611 | 30 | 33 | 2.85e−006 |
| 792 | 21 | 160 | 379, 382 | hsa-miR-365 | 1.89 | 759 | 1431 | 30 | 33 | 1.24e−007 |
| 793 | 21 | 128 | 294 | hsa-miR-370 | 1.38 | 790 | 1091 | 30 | 33 | 0.0359 |
| 794 | 21 | 51 | 497 | hsa-miR-423 | 1.35 | 2387 | 3231 | 30 | 33 | 0.00427 |
| 795 | 21 | 62 | 485 | hsa-miR-489 | 1.38 | 407 | 559 | 30 | 33 | 0.0129 |
| 796 | 21 | 63 | 557 | hsa-miR-491 | 1.53 | 2495 | 3822 | 30 | 33 | 0.0298 |
| 797 | 21 | 43 | 418 | hsa-miR-504 | 1.4 | 921 | 1285 | 30 | 33 | 0.00896 |
| 798 | 21 | 258 | 428, 443 | hsa-miR-513 | 1.42 | 1264 | 1799 | 30 | 33 | 0.0275 |
| 799 | 21 | 210 | 400 | hsa-miR-518c* | 1.44 | 769 | 1107 | 30 | 33 | 0.00547 |
| 800 | 21 | 102 | 344 | hsa-miR-520a* | 1.23 | 437 | 538 | 30 | 33 | 0.0158 |
| 801 | 21 | 206 | 513 | hsa-miR-520d* | 1.56 | 607 | 948 | 30 | 33 | 0.0145 |
| 802 | 21 | 100 | 524 | hsa-miR-524* | 1.29 | 607 | 782 | 30 | 33 | 0.0474 |
| 803 | 21 | 56 | 338 | hsa-miR-564 | 1.69 | 314 | 530 | 30 | 33 | 0.00351 |
| 804 | 21 | 12 | 275 | hsa-miR-634 | 1.93 | 368 | 711 | 30 | 33 | 0.000157 |
| 805 | 21 | 182 | 304, 317 | hsa-miR-92 | 1.35 | 2271 | 1687 | 30 | 33 | 0.00487 |
| 806 | 22 | 127 | 361 | 11_1 | 1.57 | 526 | 828 | 7 | 5 | 0.0277 |
| 807 | 22 | 121 | 502 | 70_14 | 2.1 | 428 | 900 | 7 | 5 | 0.00396 |
| 808 | 22 | 87 | 301 | gam11 | 20.39 | 9897 | 485 | 7 | 5 | 0.0286 |
| 809 | 22 | 125 | 532 | 70_16 | 4.49 | 719 | 160 | 7 | 5 | 0.031 |
| 810 | 22 | 209 | 507 | hsa-miR-150 | 1.62 | 876 | 539 | 7 | 5 | 0.0407 |
| 811 | 22 | 205 | 367 | hsa-miR-187 | 1.75 | 305 | 534 | 7 | 5 | 0.0363 |
| 812 | 22 | 16 | 488 | hsa-miR-193b | 6.33 | 1118 | 177 | 7 | 5 | 0.0209 |
| 813 | 22 | 156 | 446 | hsa-miR-200c | 1.78 | 6973 | 3923 | 7 | 5 | 0.0311 |
| 814 | 22 | 175 | 462 | hsa-miR-21 | 2.44 | 5527 | 2266 | 7 | 5 | 0.0432 |
| 815 | 22 | 66 | 306 | hsa-miR-23b | 89.38 | 4499 | 50 | 7 | 5 | 0.000545 |
| 816 | 22 | 238 | 346, 416 | hsa-miR-24 | 2.32 | 7696 | 17830 | 7 | 5 | 0.0283 |
| 817 | 22 | 90 | 388 | hsa-miR-25 | 4.71 | 787 | 167 | 7 | 5 | 0.0325 |
| 818 | 22 | 167 | 556 | hsa-miR-29a | 34.72 | 1748 | 50 | 7 | 5 | 0.000574 |
| 819 | 22 | 244 | 287 | hsa-miR-30a-5p | 12.73 | 641 | 50 | 7 | 5 | 0.0103 |
| 820 | 22 | 241 | 550 | hsa-miR-30d | 1.58 | 2467 | 1564 | 7 | 5 | 0.0374 |
| 821 | 22 | 245 | 431 | hsa-miR-30e-5p | 1.8 | 3224 | 1790 | 7 | 5 | 0.0454 |
| 822 | 22 | 237 | 480 | hsa-miR-34a | 1.67 | 1054 | 1762 | 7 | 5 | 0.0443 |
| 823 | 22 | 255 | 444 | hsa-miR-361 | 5.16 | 828 | 160 | 7 | 5 | 0.0222 |
| 824 | 23 | 127 | 361 | 11_1 | 1.69 | 428 | 723 | 7 | 6 | 0.0137 |
| 825 | 23 | 7 | 337 | 43_21 | 1.48 | 357 | 529 | 7 | 6 | 0.0498 |
| 826 | 23 | 101 | 475 | 43_30 | 1.41 | 383 | 542 | 7 | 6 | 0.0487 |
| 827 | 23 | 121 | 502 | 70_14 | 2.34 | 352 | 823 | 7 | 6 | 0.00277 |
| 828 | 23 | 125 | 532 | 70_16 | 4.85 | 576 | 119 | 7 | 6 | 0.0097 |
| 829 | 23 | 220 | 475 | hsa-let-7e | 1.43 | 3735 | 2620 | 7 | 6 | 0.00752 |
| 830 | 23 | 153 | 445 | hsa-miR-132 | 1.66 | 329 | 547 | 7 | 6 | 0.0154 |
| 831 | 23 | 180 | 486 | hsa-miR-135b | 1.94 | 306 | 595 | 7 | 6 | 0.027 |
| 832 | 23 | 10 | 371, 461 | hsa-miR-181a | 1.58 | 703 | 1109 | 7 | 6 | 0.0312 |
| 833 | 23 | 234 | 450 | hsa-miR-185 | 1.55 | 487 | 758 | 7 | 6 | 0.00957 |
| 834 | 23 | 271 | 334 | hsa-miR-202* | 1.64 | 523 | 858 | 7 | 6 | 0.0385 |
| 835 | 23 | 66 | 306 | hsa-miR-23b | 17.06 | 3682 | 216 | 7 | 6 | 0.032 |
| 836 | 23 | 90 | 388 | hsa-miR-25 | 5.33 | 627 | 118 | 7 | 6 | 0.0069 |
| 837 | 23 | 257 | 409 | hsa-miR-26b | 4.42 | 923 | 209 | 7 | 6 | 0.0301 |
| 838 | 23 | 167 | 556 | hsa-miR-29a | 10.26 | 1403 | 137 | 7 | 6 | 0.018 |
| 839 | 23 | 229 | 292 | hsa-miR-345 | 1.55 | 685 | 1060 | 7 | 6 | 0.0301 |
| 840 | 23 | 237 | 480 | hsa-miR-34a | 1.98 | 834 | 1648 | 7 | 6 | 0.013 |
| 841 | 23 | 255 | 444 | hsa-miR-361 | 3.5 | 660 | 189 | 7 | 6 | 0.0455 |
| 842 | 23 | 128 | 294 | hsa-miR-370 | 1.83 | 654 | 1201 | 7 | 6 | 0.0462 |
| 843 | 23 | 148 | 283 | hsa-miR-557 | 1.75 | 395 | 690 | 7 | 6 | 0.0271 |
| 844 | 23 | 135 | 315 | hsa-miR-565 | 1.98 | 2700 | 5336 | 7 | 6 | 0.00992 |
| 845 | 23 | 80 | 509 | hsa-miR-99b | 1.49 | 1023 | 1527 | 7 | 6 | 0.0261 |
| 846 | 24 | 225 | 332 | hsa-let-7b | 1.36 | 25887 | 35272 | 6 | 5 | 0.0217 |
| 847 | 24 | 151 | 472 | hsa-miR-141 | 2.13 | 6447 | 3021 | 6 | 5 | 0.035 |
| 848 | 24 | 156 | 446 | hsa-miR-200c | 1.75 | 7826 | 4465 | 6 | 5 | 0.0123 |
| 849 | 24 | 241 | 550 | hsa-miR-30d | 1.34 | 2404 | 1790 | 6 | 5 | 0.0395 |
| 850 | 25 | 196 | 508 | 15_rel | 1.5 | 38287 | 57277 | 6 | 7 | 0.0114 |
| 851 | 25 | 219 | 497 | 27_3 | 1.82 | 924 | 1682 | 6 | 7 | 0.00912 |
| 852 | 25 | 129 | 548 | 43_39 | 2.26 | 295 | 667 | 6 | 7 | 0.0133 |
| 853 | 25 | 145 | 447 | 43_44_rel | 2.13 | 38553 | 82305 | 6 | 7 | 0.000101 |
| 854 | 25 | 183 | 373 | 66_1 | 2.49 | 232 | 578 | 6 | 7 | 0.0147 |
| 855 | 25 | 177 | 366, 464 | 72_12 | 2.81 | 3715 | 1321 | 6 | 7 | 0.000903 |
| 856 | 25 | 125 | 532 | 70_16 | 4.73 | 1418 | 300 | 6 | 7 | 1.03e−006 |
| 857 | 25 | 223 | 320, 393, 559 | hsa-let-7a | 4.12 | 3187 | 774 | 6 | 7 | 0.0119 |
| 858 | 25 | 224 | 427 | hsa-let-7c | 3.44 | 14110 | 4102 | 6 | 7 | 0.0125 |
| 859 | 25 | 13 | 397 | hsa-miR-100 | 2.57 | 3716 | 1444 | 6 | 7 | 0.00965 |
| 860 | 25 | 48 | 483, 529 | hsa-miR-103 | 2.87 | 2966 | 8502 | 6 | 7 | 0.0131 |
| 861 | 25 | 47 | 555 | hsa-miR-107 | 2.64 | 2543 | 6715 | 6 | 7 | 0.0212 |
| 862 | 25 | 198 | 313, 551 | hsa-miR-125b | 3.44 | 21890 | 6361 | 6 | 7 | 9.37e−006 |
| 863 | 25 | 266 | 295, 386 | hsa-miR-133a | 2.29 | 239 | 549 | 6 | 7 | 0.0275 |
| 864 | 25 | 151 | 472 | hsa-miR-141 | 26.15 | 165 | 4321 | 6 | 7 | 1.27e−006 |
| 865 | 25 | 218 | 527 | hsa-miR-143 | 2.21 | 38298 | 17359 | 6 | 7 | 0.0245 |
| 866 | 25 | 212 | 333 | hsa-miR-149 | 3.09 | 1316 | 426 | 6 | 7 | 0.00114 |
| 867 | 25 | 172 | 329 | hsa-miR-15b | 1.62 | 887 | 547 | 6 | 7 | 0.0228 |
| 868 | 25 | 10 | 371, 461 | hsa-miR-181a | 3.39 | 4480 | 1320 | 6 | 7 | 7.27e−005 |
| 869 | 25 | 11 | 456, 540 | hsa-miR-181b | 1.98 | 952 | 482 | 6 | 7 | 0.00262 |
| 870 | 25 | 272 | 323 | hsa-miR-182 | 9.01 | 132 | 1188 | 6 | 7 | 0.00694 |
| 871 | 25 | 234 | 450 | hsa-miR-185 | 1.51 | 598 | 905 | 6 | 7 | 0.0218 |
| 872 | 25 | 109 | 374 | hsa-miR-192 | 12.74 | 196 | 2496 | 6 | 7 | 7.83e−005 |
| 873 | 25 | 17 | 442 | hsa-miR-193a | 2.6 | 870 | 2262 | 6 | 7 | 0.00089 |
| 874 | 25 | 16 | 488 | hsa-miR-193b | 3.61 | 1796 | 498 | 6 | 7 | 0.00167 |
| 875 | 25 | 246 | 290, 549 | hsa-miR-194 | 29.36 | 296 | 8702 | 6 | 7 | 9.23e−006 |
| 876 | 25 | 92 | 410, 432 | hsa-miR-199a | 2.2 | 7549 | 3429 | 6 | 7 | 0.000331 |
| 877 | 25 | 152 | 369 | hsa-miR-200a | 16.12 | 215 | 3464 | 6 | 7 | 1.66e−005 |
| 878 | 25 | 157 | 411 | hsa-miR-200b | 20.2 | 157 | 3167 | 6 | 7 | 2.35e−005 |
| 879 | 25 | 156 | 446 | hsa-miR-200c | 43.49 | 199 | 8644 | 6 | 7 | 1.19e−006 |
| 880 | 25 | 238 | 346, 416 | hsa-miR-24 | 1.48 | 10992 | 16278 | 6 | 7 | 0.015 |
| 881 | 25 | 260 | 280 | hsa-miR-27b | 1.97 | 945 | 1863 | 6 | 7 | 0.0217 |
| 882 | 25 | 61 | 286 | hsa-miR-296 | 1.87 | 965 | 1809 | 6 | 7 | 0.0382 |
| 883 | 25 | 244 | 287 | hsa-miR-30a-5p | 4.8 | 5019 | 1045 | 6 | 7 | 0.000188 |
| 884 | 25 | 243 | 299, 316 | hsa-miR-30c | 7.06 | 2593 | 367 | 6 | 7 | 0.0344 |
| 885 | 25 | 132 | 429 | hsa-miR-31 | 2.58 | 538 | 1388 | 6 | 7 | 0.048 |
| 886 | 25 | 98 | 368 | hsa-miR-324-5p | 2.23 | 640 | 1426 | 6 | 7 | 0.0416 |
| 887 | 25 | 208 | 477 | hsa-miR-342 | 2.02 | 646 | 1307 | 6 | 7 | 0.00218 |
| 888 | 25 | 255 | 444 | hsa-miR-361 | 1.32 | 601 | 793 | 6 | 7 | 0.0333 |
| 889 | 25 | 273 | 335 | hsa-miR-375 | 117.37 | 117 | 13762 | 6 | 7 | 6.68e−008 |
| 890 | 25 | 158 | 378 | hsa-miR-429 | 4.41 | 138 | 608 | 6 | 7 | 0.00317 |
| 891 | 25 | 62 | 485 | hsa-miR-489 | 5.26 | 131 | 689 | 6 | 7 | 4.72e−005 |
| 892 | 25 | 73 | 503 | hsa-miR-518b | 2.13 | 259 | 552 | 6 | 7 | 0.0393 |
| 893 | 25 | 21 | 517, 519 | hsa-miR-520f | 3.29 | 530 | 161 | 6 | 7 | 0.000293 |
| 894 | 25 | 230 | 474, 484, 536 | hsa-miR-7 | 3.28 | 225 | 738 | 6 | 7 | 0.0062 |
| 895 | 25 | 14 | 318 | hsa-miR-99a | 3.2 | 4137 | 1293 | 6 | 7 | 2.5e−005 |
| 896 | 26 | 94 | 394 | 69_6_rel | 1.82 | 2813 | 5110 | 17 | 11 | 0.0284 |
| 897 | 26 | 33 | 339, 552 | 96_rel | 1.75 | 23341 | 40841 | 14 | 7 | 0.000544 |
| 898 | 26 | 225 | 332 | hsa-let-7b | 1.63 | 10262 | 6291 | 17 | 11 | 0.0305 |
| 899 | 26 | 45 | 277 | hsa-let-7d | 1.39 | 1758 | 1262 | 17 | 11 | 0.0493 |
| 900 | 26 | 220 | 475 | hsa-let-7e | 1.65 | 2469 | 1495 | 17 | 11 | 0.00382 |
| 901 | 26 | 2 | 399 | hsa-miR-106a | 1.49 | 931 | 1389 | 17 | 11 | 0.0355 |
| 902 | 26 | 47 | 555 | hsa-miR-107 | 1.68 | 2575 | 1533 | 17 | 11 | 0.0368 |
| 903 | 26 | 199 | 387 | hsa-miR-125a | 1.72 | 2837 | 1645 | 17 | 11 | 0.0337 |
| 904 | 26 | 198 | 313, 551 | hsa-miR-125b | 1.91 | 6405 | 3350 | 17 | 11 | 0.00943 |
| 905 | 26 | 204 | 419 | hsa-miR-126 | 1.56 | 896 | 1395 | 17 | 11 | 0.0207 |
| 906 | 26 | 266 | 295, 386 | hsa-miR-133a | 2.17 | 287 | 623 | 17 | 11 | 0.000637 |
| 907 | 26 | 265 | 533 | hsa-miR-133b | 2.23 | 231 | 514 | 17 | 11 | 0.0018 |
| 908 | 26 | 151 | 472 | hsa-miR-141 | 3.05 | 1102 | 3361 | 17 | 11 | 0.0378 |
| 909 | 26 | 209 | 507 | hsa-miR-150 | 2.08 | 434 | 902 | 17 | 11 | 0.000596 |
| 910 | 26 | 75 | 467 | hsa-miR-17-5p | 1.61 | 702 | 1128 | 17 | 11 | 0.0175 |
| 911 | 26 | 109 | 374 | hsa-miR-192 | 5.3 | 640 | 3393 | 17 | 11 | 0.000668 |
| 912 | 26 | 246 | 290, 549 | hsa-miR-194 | 7.61 | 1416 | 10777 | 17 | 11 | 0.00069 |
| 913 | 26 | 61 | 286 | hsa-miR-296 | 1.54 | 1013 | 1563 | 17 | 11 | 0.0425 |
| 914 | 26 | 132 | 429 | hsa-miR-31 | 5.66 | 698 | 3952 | 17 | 11 | 1.64e−006 |
| 915 | 26 | 91 | 368 | hsa-miR-324-3p | 1.98 | 873 | 441 | 17 | 11 | 0.0223 |
| 916 | 26 | 185 | 492 | hsa-miR-335 | 4.33 | 334 | 1446 | 17 | 11 | 1.92e−005 |
| 917 | 26 | 237 | 480 | hsa-miR-34a | 1.31 | 1217 | 930 | 17 | 11 | 0.0441 |
| 918 | 26 | 255 | 444 | hsa-miR-361 | 1.25 | 524 | 421 | 17 | 11 | 0.0163 |
| 919 | 26 | 111 | 389 | hsa-miR-422a | 1.91 | 266 | 509 | 17 | 11 | 5.77e−006 |
| 920 | 26 | 112 | 372 | hsa-miR-422b | 2.49 | 713 | 1777 | 17 | 11 | 0.000388 |
| 921 | 26 | 214 | 376 | hsa-miR-494 | 2.35 | 3440 | 8082 | 17 | 11 | 0.0446 |
| 922 | 26 | 14 | 318 | hsa-miR-99a | 2.05 | 1476 | 721 | 17 | 11 | 0.00981 |
| 923 | 26 | 80 | 509 | hsa-miR-99b | 1.72 | 1210 | 702 | 17 | 11 | 0.00626 |
| 924 | 27 | 197 | 444 | 25_6 | 1.59 | 995 | 626 | 10 | 5 | 0.0322 |
| 925 | 27 | 101 | 475 | 43_30 | 1.95 | 601 | 308 | 10 | 5 | 0.00118 |
| 926 | 27 | 145 | 447 | 43_44_rel | 1.42 | 70143 | 49426 | 10 | 5 | 0.00194 |
| 927 | 27 | 34 | 396 | hsa-miR-151 | 1.25 | 822 | 656 | 10 | 5 | 0.0207 |
| 928 | 27 | 130 | 440 | hsa-miR-191* | 2.67 | 1906 | 713 | 10 | 5 | 0.0034 |
| 929 | 27 | 17 | 442 | hsa-miR-193a | 2.12 | 3246 | 6886 | 10 | 5 | 0.034 |
| 930 | 27 | 159 | 494 | hsa-miR-216 | 1.71 | 728 | 426 | 10 | 5 | 0.0134 |
| 931 | 27 | 61 | 286 | hsa-miR-296 | 2.22 | 3075 | 1387 | 10 | 5 | 0.0124 |
| 932 | 27 | 160 | 379, 382 | hsa-miR-365 | 1.58 | 799 | 507 | 10 | 5 | 0.00423 |
| 933 | 27 | 73 | 503 | hsa-miR-518b | 1.6 | 576 | 360 | 10 | 5 | 0.0248 |
| 934 | 27 | 36 | 385 | hsa-miR-766 | 3.29 | 1054 | 320 | 10 | 5 | 0.00419 |
| 935 | 27 | 6 | 471 | hsa-miR-93 | 1.31 | 2206 | 1687 | 10 | 5 | 0.00539 |
| 936 | 28 | 27 | 313 | 43_40 | 1.97 | 321 | 631 | 5 | 6 | 0.00496 |
| 937 | 28 | 94 | 394 | 69_6_rel | 2.41 | 2372 | 983 | 5 | 6 | 0.0372 |
| 938 | 28 | 177 | 366, 464 | 72_12 | 1.94 | 3064 | 1576 | 5 | 6 | 0.0489 |
| 939 | 28 | 87 | 301 | gam11 | 3.99 | 20247 | 5068 | 5 | 6 | 0.000745 |
| 940 | 28 | 125 | 532 | 70_16 | 2.05 | 305 | 626 | 5 | 6 | 0.0248 |
| 941 | 28 | 163 | 402 | 43_7 | 6.26 | 169 | 1056 | 5 | 6 | 0.000894 |
| 942 | 28 | 224 | 427 | hsa-let-7c | 1.41 | 10628 | 14961 | 5 | 6 | 0.046 |
| 943 | 28 | 13 | 397 | hsa-miR-100 | 4.7 | 640 | 3010 | 5 | 6 | 0.000912 |
| 944 | 28 | 149 | 392 | hsa-miR-106b | 1.97 | 1081 | 548 | 5 | 6 | 0.00761 |
| 945 | 28 | 165 | 340 | hsa-miR-10b | 3.44 | 289 | 993 | 5 | 6 | 0.0379 |
| 946 | 28 | 199 | 387 | hsa-miR-125a | 2.47 | 2744 | 6779 | 5 | 6 | 0.000151 |
| 947 | 28 | 198 | 313, 551 | hsa-miR-125b | 3.77 | 5980 | 22552 | 5 | 6 | 0.00541 |
| 948 | 28 | 204 | 419 | hsa-miR-126 | 2.91 | 818 | 2377 | 5 | 6 | 0.0404 |
| 949 | 28 | 151 | 472 | hsa-miR-141 | 29.36 | 5478 | 187 | 5 | 6 | 0.00195 |
| 950 | 28 | 218 | 527 | hsa-miR-143 | 4.23 | 5434 | 22986 | 5 | 6 | 0.0336 |
| 951 | 28 | 139 | 281 | hsa-miR-145 | 6.51 | 3649 | 23770 | 5 | 6 | 0.00949 |
| 952 | 28 | 172 | 329 | hsa-miR-15b | 2.02 | 1327 | 658 | 5 | 6 | 0.0174 |
| 953 | 28 | 173 | 360, 468 | hsa-miR-16 | 1.97 | 4621 | 2345 | 5 | 6 | 0.00922 |
| 954 | 28 | 77 | 440 | hsa-miR-191 | 2.41 | 3429 | 1425 | 5 | 6 | 0.011 |
| 955 | 28 | 17 | 442 | hsa-miR-193a | 1.69 | 2342 | 3953 | 5 | 6 | 0.0391 |
| 956 | 28 | 170 | 327 | hsa-miR-195 | 2.64 | 966 | 2554 | 5 | 6 | 0.00486 |
| 957 | 28 | 92 | 410, 432 | hsa-miR-199a | 2.96 | 2182 | 6464 | 5 | 6 | 0.0458 |
| 958 | 28 | 152 | 369 | hsa-miR-200a | 15.22 | 5561 | 365 | 5 | 6 | 0.000881 |
| 959 | 28 | 157 | 411 | hsa-miR-200b | 26.2 | 5519 | 211 | 5 | 6 | 0.00088 |
| 960 | 28 | 156 | 446 | hsa-miR-200c | 23.27 | 10530 | 453 | 5 | 6 | 0.00103 |
| 961 | 28 | 203 | 504 | hsa-miR-205 | 13.57 | 1161 | 86 | 5 | 6 | 0.000945 |
| 962 | 28 | 175 | 462 | hsa-miR-21 | 3.38 | 8281 | 2449 | 5 | 6 | 0.01 |
| 963 | 28 | 115 | 314 | hsa-miR-210 | 3.14 | 674 | 214 | 5 | 6 | 0.00376 |
| 964 | 28 | 50 | 364 | hsa-miR-221 | 2.54 | 1204 | 3059 | 5 | 6 | 0.0145 |
| 965 | 28 | 49 | 302 | hsa-miR-222 | 2.75 | 1628 | 4480 | 5 | 6 | 0.0105 |
| 966 | 28 | 256 | 441, 487 | hsa-miR-26a | 1.66 | 7107 | 11775 | 5 | 6 | 0.0487 |
| 967 | 28 | 260 | 280 | hsa-miR-27b | 2.77 | 1411 | 3905 | 5 | 6 | 0.00922 |
| 968 | 28 | 167 | 556 | hsa-miR-29a | 2.31 | 1664 | 3852 | 5 | 6 | 0.00889 |
| 969 | 28 | 132 | 429 | hsa-miR-31 | 4.64 | 519 | 112 | 5 | 6 | 0.028 |
| 970 | 28 | 194 | 337 | hsa-miR-338 | 1.49 | 1792 | 2665 | 5 | 6 | 0.0028 |
| 971 | 28 | 237 | 480 | hsa-miR-34a | 1.83 | 1630 | 889 | 5 | 6 | 0.0236 |
| 972 | 28 | 236 | 413 | hsa-miR-449 | 9.17 | 3659 | 399 | 5 | 6 | 0.00979 |
| 973 | 28 | 58 | 465 | hsa-miR-449b | 14.44 | 1657 | 115 | 5 | 6 | 0.0338 |
| 974 | 28 | 63 | 557 | hsa-miR-491 | 2.8 | 1050 | 375 | 5 | 6 | 0.00567 |
| 975 | 28 | 83 | 312 | hsa-miR-497 | 2.23 | 1428 | 3182 | 5 | 6 | 0.00211 |
| 976 | 28 | 135 | 315 | hsa-miR-565 | 2.56 | 2294 | 898 | 5 | 6 | 0.00309 |
| 977 | 28 | 53 | 459 | hsa-miR-613 | 9.41 | 663 | 71 | 5 | 6 | 0.00982 |
| 978 | 28 | 6 | 471 | hsa-miR-93 | 2.57 | 2065 | 802 | 5 | 6 | 0.0169 |
| 979 | 28 | 14 | 318 | hsa-miR-99a | 4.33 | 2939 | 12728 | 5 | 6 | 0.00149 |

TABLE 4

| DID | Decision_name | Tissue1_name | Tissue2_name |
|---|---|---|---|
| 1 | Bladder-cancer | Bladder-Tumor | Bladder-Normal |
| 2 | Bladder-high-low-grades | Bladder-High-grade | Bladder-Low- |
| | | | grade |
| 3 | Bladder-high-med-grades | Bladder-High-grade | Bladder-Med- |
| | | | grade |
| 4 | Bladder-med-low-grades | Bladder-Med-grade | Bladder-Low- |
| | | | grade |
| 5 | Breast-cancer | Breast-Tumor | Breast-Normal |
| 6 | Breast-high-med-grades | Breast-High-grade | Breast-Med-grade |
| 7 | Colon-cancer | Colon-Tumor | Colon-Normal |
| 8 | Colon-med-low-grades | Colon-Med-grade | Colon-Low-grade |
| 9 | Kidney-cancer | Kidney-Tumor | Kidney-Normal |
| 10 | Liver-cancer | Liver-Tumor | Liver-Normal |
| 11 | Liver-cancer-Metast. | Liver-Primary | Liver-Metastasis |
| 12 | Lung-Adeno-Large | Lung-Adeno | Lung-Large |
| 13 | Lung-Adeno-Squamous | Lung-Adeno | Lung-Squamous |
| 14 | Lung-Large-Squamous | Lung-Large | Lung-Squamous |
| 15 | Lung-cancer | Lung-Tumor | Lung-Normal |
| 16 | Lung-high-low-grades | Lung-High-grade | Lung-Low-grade |
| 17 | Lung-high-med-grades | Lung-High-grade | Lung-Med-grade |
| 18 | Lung-med-low-grades | Lung-Med-grade | Lung-Low-grade |
| 19 | Ovary-cancer | Ovary-Tumor | Ovary-Normal |
| 20 | Prostate-Adeno-BPH | Prostate- | Prostate-BPH |
| | | Adenocarcinoma | |
| 21 | Prostate-cancer | Prostate-Tumor | Prostate-Normal |
| 22 | Prostate-high-low-grades | Prostate-High-grade | Prostate-Low- |
| | | | grade |
| 23 | Prostate-high-med-grades | Prostate-High-grade | Prostate-Med- |
| | | | grade |
| 24 | Prostate-med-low-grades | Prostate-Med-grade | Prostate-Low- |
| | | | grade |
| 25 | Small intestine-Subtypes | Small intestine- | Small intestine- |
| | | Stromal | Carcinoid |
| 26 | Small intestine-cancer | Small intestine- | Small intestine- |
| | | Tumor | Normal |
| 27 | Thyroid-cancer | Thyroid-Tumor | Thyroid- |
| | | | Normal |
| 28 | Uterus-cancer | Uterus-Tumor | Uterus-Normal |

1.-16. (canceled)

17. A method to distinguish between small intestine carcinoid tumor and small intestine stromal tumor, the method comprising determining a level of a nucleic acid sequence selected from the group consisting of SEQ ID NO. 273, 10, 11, 13, 14, 16, 17, 21, 47, 48, 61, 62, 73, 92, 98, 109, 125, 129, 132, 145, 151, 152, 156-158, 172, 177, 183, 196, 198, 208, 212, 218, 219, 223, 224, 230, 234, 238, 243, 244, 246, 255, 260, 266, 272 and sequences at least about 81% identical thereto in a small intestine sample.

18. A method to distinguish between lung adenocarcinoma and squamous cell carcinoma, the method comprising determining a level of a nucleic acid sequence selected from the group consisting of SEQ ID NO. 203, 33, 44, 50, 64, 87, 90, 92, 94, 97, 100, 111, 112, 116, 120, 135, 136, 152, 153, 156, 169, 182, 199, 204, 226, 237, 238, 256-258, 267 and sequences at least about 81% identical thereto in a lung sample.

19. A method to distinguish between lung undifferentiated large cell carcinoma and squamous cell carcinoma, the method comprising determining a level of a nucleic acid sequence selected from the group consisting of SEQ ID NO. 6, 14, 16, 29, 35, 48, 51, 54, 60, 61, 63, 66, 67, 77, 86, 90, 92, 113, 115, 135, 139, 141, 151, 152, 156, 157, 179, 203, 204, 218, 220, 224, 225, 236, 237, 241, 244, 259 and sequences at least about 81% identical thereto in a lung sample.

20. A method to distinguish between prostate adenocarcinoma and benign prostatic hyperplasia (BPH), the method comprising determining a level of a nucleic acid sequence selected from the group consisting of SEQ ID NO. 1, 44, 52, 75, 90, 107, 118, 149, 158, 173, 191, 192, 204, 212, 214, 218, 230, 251, 253, 259 and sequences at least about 81% identical thereto in a prostate sample.

21. A method to distinguish between high grade carcinoma, medium grade carcinoma and low grade carcinoma, the method comprising determining a level of a nucleic acid sequence selected from the group consisting of SEQ ID NO: 1, 4, 6-10, 13, 14, 16, 18, 19, 27, 29, 34, 35, 37, 38, 40, 42, 43, 52-45, 57, 60, 61, 63, 66-68, 73, 75, 77, 80, 83, 84, 86, 87, 92-89, 94, 97-102, 110-113, 115, 117, 120-122, 125, 127-130, 132, 134-136, 139, 140, 143, 144, 146, 148-151, 153, 155-159, 161-167, 169-173, 175, 177, 179, 180, 183-185, 187-191, 193, 194, 196, 198-200, 203-206, 209, 213-215, 218-220, 222-226, 229-234, 236-238, 241-246, 248, 251, 254, 255-257, 259, 262, 265-269, 271, 272, 274 and sequences at least about 81% identical thereto in a carcinoma sample.

22. The method of claim 21 wherein said carcinoma is selected from the group consisting of bladder carcinoma, breast carcinoma, colon carcinoma, lung carcinoma and prostate carcinoma.

23. A method to distinguish between primary liver cancer and liver metastasis, the method comprising determining a level of a nucleic acid sequence selected from the group consisting of SEQ ID NO. 1, 2, 4, 6, 9, 13, 14, 17, 18, 31, 33-37, 42, 44, 46-50, 52, 54, 60, 63, 64, 73, 77, 83, 97, 101, 104, 105, 113, 114, 121, 123, 125, 126, 128, 132, 141, 145, 146, 149, 151, 152, 156, 157, 163, 168, 179, 183, 194, 197-200, 204, 206, 208, 210, 213, 214, 220, 221, 226-228, 230, 232, 234-238, 240-243, 245, 247-250, 256, 258, 260, 264, 266, 273 in a liver sample.

24. A kit for diagnosing a subject with a specific cancer, said kit comprising a probe comprising a sequence selected from the group consisting of a sequence that is complementary to a sequence selected from SEQ ID NOs: 1-561, and a sequence having at least about 81% identity thereto.

25. The kit of claim 24, wherein the specific cancer is selected from a group consisting of small intestine carcinoid tumor, small intestine stromal tumor, lung adenocarcinoma, squamous cell carcinoma, lung undifferentiated large cell carcinoma, squamous cell carcinoma, prostate adenocarcinoma and benign prostatic hyperplasia (BPH), high grade carcinoma, medium grade carcinoma and low grade carcinoma, primary liver cancer and liver metastasis.
